(19) United States
(12) Patent Application Publication
(10) Pub. No.: US 2010/0021473 A1
(43) Pub. Date: Jan. 28, 2010

(54) Bispecific Ligands With Binding Specificity to Cell Surface Targets and Methods of Use Therefor

(75) Inventors: Elena De Angelis, Cambridgeshire (GB); Steve Holmes, Cambridgeshire (GB); Ian Tomlinson, Hertfordshire (GB); Eric Yi-Chun Huang, Cambridgeshire (GB); Lucy J. Holt, Cambridgeshire (GB); Claire E. Everett, Cambridgeshire (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: DOMANTIS LIMITED, Cambridge (GB)

(21) Appl. No.: 12/086,115

(22) PCT Filed: Dec. 5, 2006
(86) PCT No.: PCT/GB2006/004565

§ 371 Date: Oct. 5, 2009

Related U.S. Application Data
(60) Provisional application No. 60/742,992, filed on Dec. 6, 2005.

(51) Int. Cl. A61K 39/395; C07K 16/00; C12N 5/00; C07H 21/04; C12N 15/63; C12P 21/06; A61P 35/04

(52) U.S. Cl. 424/158.1; 530/389.2; 530/391.3; 530/391.7; 435/325; 536/23.53; 435/320.1; 435/69.1

(57) ABSTRACT

Disclosed are ligands comprising a first polypeptide domain having a binding site with binding specificity for a first cell surface target and a second polypeptide domain having a binding site for a second cell surface target, wherein each target are different and on the same cell. In some embodiments, the ligands described further comprise a toxin. In other embodiments, the ligands further comprise half-life extending moieties. Also disclosed are methods of using these ligands. In particular, the use of these ligands for cancer therapy is described.

### RELATED APPLICATION

This application claims the benefit of U.S. Application No. 60/742,992, filed Dec. 6, 2005, the entire teachings of which are incorporated herein by reference.

### BACKGROUND OF THE INVENTION

An approach to cancer therapy and diagnosis involves directing antibodies or antibody fragments to disease tissues, wherein the antibody or antibody fragment can target a diagnostic agent or therapeutic agent to the disease site. Pathogenic cells such as cancer cells have been shown to overexpress certain targets or express different targets when compared to normal cells. For example, in multiple myeloma, a B cell malignancy characterized by proliferation of plasma cells in the bone marrow, the antigens CD38, CD138 and CD56 are all highly expressed. Antibodies that bind these targets are useful in cancer therapy and diagnosis.

HERCEPTIN® (Trastuzumab) and RITUXAN® (rituximab) (both from Genentech, S. San Francisco), have been used successfully to treat breast cancer and non-Hodgkin's lymphoma, respectively. RITUXAN® is a genetically engineered chimeric murine/human monoclonal antibody directed against the CD20. HERCEPTIN® is a recombinant DNA-derived humanized monoclonal antibody that selectively binds to the extracellular domain of the human epidermal growth factor receptor 2 (HER2) proto-oncogene. The Herceptin target, HER-2/neu, also known as c-erb B-2, is a 185 kDa transmembrane receptor with protein tyrosine kinase activity that is a member of the epithelial growth factor (EGF) receptor family expressed on the breast, ovarian, gastric and prostatic tumors of subsets of patients with these disorders. This receptor is modestly expressed in normal adult tissues; however, it is strongly associated with the epithelial solid malignancies and is overexpressed in approximately 25-35% of human gastric, lung, prostatic and breast carcinomas.

Current therapies, including monoclonal antibodies, typically address singularly defined targets that are different throughout a population or change, evolve and mutate during the spread of disease throughout a population or within an individual. Additionally, a single antibody or domain will probably not recognize all the tumor cells in a patient, but combinations of antibodies or domains may be significantly more effective. Furthermore, crossreactivity can be a problem with antibodies. One of the major drawbacks of the use of anti-CEA antibodies for clinical purposes has been the cross-reactivity of these antibodies with some apparently normal adult tissues. Previous studies have shown that most conventional hyperimmune antisera raised against different immunogenic forms of CEA cross-react with CEA-related antigens found in normal colonic mucosa, spleen, liver, lung, sweat glands, polymorphonuclear leukocytes and monocytes of normal individuals, as well as many different types of carcinomas.

Thus, a need exists for improved agents for treating pathogenic conditions (e.g., cancer).

### SUMMARY OF THE INVENTION

The invention relates to ligands that bind two cell surface targets that are present on a cell. For example, the ligand can comprise a first polypeptide domain having a binding site with binding specificity for a first cell surface target and a second polypeptide domain having a binding site with binding specificity for a second cell surface target. Preferably, the first polypeptide domain (e.g., immunoglobulin single variable domain) binds said first cell surface target with low affinity and said second polypeptide domain (immunoglobulin single variable domain) binds said second cell surface target with low affinity.

As described and exemplified herein, such ligands can selectively bind to double positive cells that contain both the first cell surface target and the second cell surface target. Accordingly, polypeptides that bind a desired cell surface antigen with low affinity, such and antibodies and antigen-binding fragments of antigens, can be formatted into ligands as described herein to provide agents that can selectively bind to double positive cells.

The ligands of the invention provide several advantages. For example, as described herein, the ligands that bind two different cell surface targets can be internalized into cells upon binding the two targets on the surface of a cell. Accordingly, the ligands can be used to deliver a therapeutic agent, such as a toxin, to a double positive cell that expresses a first cell surface target and a second cell surface target, such as a cancer cell. Because the ligand can selectively bind double positive cells, possible undesirable effects that might result from delivering a therapeutic agent to a single positive cell (e.g., side effects such as immunosuppression) can be avoided using the ligands of the invention.

The ligands of the invention can bind to cell surface targets that are both present on normal cells, but that are present at higher levels on a pathogenic cell. In such circumstances, the ligand can be used to preferentially deliver a therapeutic agent (e.g., a toxin) to the pathogenic cell. For example, due to the higher level of cell surface targets on the pathogenic cell, more ligand will bind the pathogenic cell and be internalized than will bind and be internalized into the normal cell. Thus, an effective amount of toxin can be delivered preferentially to the pathogenic cell.

Further, as described herein, the ligand can be tailored to have a desired in vivo serum half-life. Thus, the ligands can be used to control, reduce, or eliminate general toxicity of therapeutic agents, such as cytotoxin used to treat cancer.

Generally both of the cell surface targets that the ligand binds are present on a pathogenic cell, but are not both present on normal cells. As shown herein, in such situations, the ligand can used at a concentration that results in selective binding to pathogenic cells that contain both cell surface targets (at a concentration wherein the ligand does not substantially bind single positive normal cells).

Certain normal cells may have both cell surface targets that are bound by a ligand of the invention present on their cell surfaces, but the targets are present at higher levels on the surface of a pathogenic cell (e.g., a cancer cell). Preferably, both cell surface targets are not substantially present on the surface of normal cells. In these circumstances, the ligand can be used at a concentration that results in selective binding to pathogenic cells that contain both cell surface targets (at a concentration wherein the ligand does not substantially bind the normal cell that contains low levels of the cell surface targets).

In one aspect, the ligand comprises a first polypeptide domain having a binding site with binding specificity for a first cell surface target and a second polypeptide domain having a binding site with binding specificity for a second cell surface target, wherein said first cell surface target and said second cell surface target are different, and said first cell surface target and said second cell surface target are present on a pathogenic cell, wherein said ligand binds said first cell surface target and said second cell surface target on said pathogenic cell, and wherein said ligand is internalized by said pathogenic cell.

Preferably, the ligand is preferentially internalized by a pathogenic cell. For example, the ligand is not substantially internalized by single positive or normal cells, or selectively binds a pathogenic cell. In some embodiments, the ligand selectively binds a pathogenic cell when said ligand is present at a concentration that is between about 1 pM and about 150 nM.

In some embodiments, the first polypeptide domain binds a first cell surface target with low affinity and the second polypeptide domain binds a second cell surface target with low affinity. For example, the first polypeptide domain and the second polypeptide domain can each bind their respective cell surface targets with an affinity (KD) that is between about 10 μM and about 10 nM, as determined by surface plasmon resonance.

In preferred embodiments, the first polypeptide domain that has a binding site with binding specificity for a first cell surface target and the second polypeptide domain that has a binding site with binding specificity for a second cell surface target are a first immunoglobulin single variable domain, and a second immunoglobulin single variable domain, respectively. For example, the first immunoglobulin single variable domain and/or the second immunoglobulin single variable domain can be a V_{HH}, or the first immunoglobulin single variable domain and the second immunoglobulin single variable domain can independently be selected from the group consisting of a human V_{H} and a human V_{L}.

In more particular embodiments, the first immunoglobulin single variable domain has a binding site with binding specificity for a cell surface target selected from the group consisting of CD38, CD 138, carcinoembrionic antigen (CEA), CD56, vascular endothelial growth factor (VEGF), epidermal growth factor receptor (EGFR), and HER2. In some embodiments, the second immunoglobulin single variable domain has a binding site with binding specificity for a cell surface target selected from the group consisting of CD38, CD138, CEA, CD56, VEGF, EGFR, and HER2, with the proviso that said first immunoglobulin single variable domain and said second immunoglobulin single variable domain do not bind the same cell surface target.

In certain embodiments, the first immunoglobulin single variable domain or the second immunoglobulin single variable domain binds CD38 and competes for binding to CD38 with an anti-CD38 domain antibody (dAb) selected from the group consisting of: DOM11-14 (SEQ ID NO: 242), DOM11-22 (SEQ ID NO:246), DOM11-23 (SEQ ID NO:247), DOM11-25 (SEQ ID NO:249), DOM11-26 (SEQ ID NO:250), DOM11-27 (SEQ ID NO:251), DOM11-29 (SEQ ID NO:253), DOM11-3 (SEQ ID NO:234), DOM11-30 (SEQ ID NO:254), DOM11-31 (SEQ ID NO:255), DOM11-32 (SEQ ID NO:256), DOM11-36 (SEQ ID NO:260), DOM11-4 (SEQ ID NO:235), DOM11-43 (SEQ ID NO:266), DOM11-44 (SEQ ID NO:267), DOM11-45 (SEQ ID NO:268), DOM11-5 (SEQ ID NO:236), DOM11-7 (SEQ ID NO:238), DOM11-1 (SEQ ID NO:232), DOM11-10 (SEQ ID NO:241), DOM11-16 (SEQ ID NO:243), DOM11-2 (SEQ ID NO:233), DOM11-20 (SEQ ID NO:244), DOM11-21 (SEQ ID NO:245), DOM11-24 (SEQ ID NO:248), DOM11-28 (SEQ ID NO:252), DOM11-33 (SEQ ID NO:257), DOM11-34 (SEQ ID NO:258), DOM11-35 (SEQ ID NO:259), DOM11-37 (SEQ ID NO:261), DOM11-38 (SEQ ID NO:262), DOM11-39 (SEQ ID NO:293), DOM11-41 (SEQ ID NO:264), DOM11-42 (SEQ ID NO:265), DOM11-6 (SEQ ID NO:237), DOM11-8 (SEQ ID NO:239), and DOM11-9 (SEQ ID NO:240).

In other embodiments, the first immunoglobulin single variable domain or the second immunoglobulin single variable domain binds CD38 and competes for binding to CD38 with an anti-CD38 domain antibody (dAb) selected from the group consisting of: DOM 11-3-1 (SEQ ID NO: 269), DOM 11-3-2 (SEQ ID NO: 270), DOM 11-3-3 (SEQ ID NO: 271), DOM 11-3-4 (SEQ ID NO: 272), DOM 11-3-6 (SEQ ID NO: 273), DOM 11-3-9 (SEQ ID NO: 274), DOM 11-3-10 (SEQ ID NO: 275), DOM 11-3-11 (SEQ ID NO: 276), DOM 11-3-14 (SEQ ID NO: 277), DOM 11-3-15 (SEQ ID NO: 278), DOM 11-3-17 (SEQ ID NO: 279), DOM 11-3-19 (SEQ ID NO: 280), DOM 11-3-20 (SEQ ID NO: 281), DOM 11-3-21 (SEQ ID NO: 282), DOM 11-3-22 (SEQ ID NO: 283), DOM 11-3-23 (SEQ ID NO: 284), DOM 11-3-24 (SEQ ID NO: 285), DOM 11-3-25 (SEQ ID NO: 286), DOM 11-3-26 (SEQ ID NO: 287), DOM 11-3-27 (SEQ ID NO: 288), DOM 11-3-28 (SEQ ID NO: 289), DOM 11-30-1 (SEQ ID NO: 290), DOM 11-30-2 (SEQ ID NO: 291), DOM 11-30-3 (SEQ ID NO: 292), DOM 11-30-5 (SEQ ID NO: 293), DOM 11-30-6 (SEQ ID NO: 294), DOM 11-30-7 (SEQ ID NO:295), DOM 11-30-8 (SEQ ID NO: 296), DOM 11-30-9 (SEQ ID NO: 297), DOM 11-30-10 (SEQ ID NO: 298), DOM 11-30-11 (SEQ ID NO: 299), DOM 11-30-12 (SEQ ID NO: 300), DOM 11-30-13 (SEQ ID NO: 301), DOM 11-30-14 (SEQ ID NO: 302), DOM 11-30-15 (SEQ ID NO: 303), DOM 11-30-16 (SEQ ID NO: 304), and DOM 11-30-17 (SEQ ID NO: 305).

In certain embodiments, the first immunoglobulin single variable domain or the second immunoglobulin single variable domain comprises an amino acid sequence that has at least about 90% amino acid sequence similarity with the amino acid sequence of a dAb selected from the group consisting of: DOM11-14 (SEQ ID NO: 242), DOM11-22 (SEQ ID NO:246), DOM11-23 (SEQ ID NO:247), DOM11-25 (SEQ ID NO:249), DOM11-26 (SEQ ID NO:250), DOM11-27 (SEQ ID NO:251), DOM 11-29 (SEQ ID NO:253), DOM11-3 (SEQ ID NO:234), DOM11-30 (SEQ ID NO:254), DOM11-31 (SEQ ID NO:255), DOM11-32 (SEQ ID NO:256), DOM11-36 (SEQ ID NO:260), DOM11-4 (SEQ ID NO:235), DOM11-43 (SEQ ID NO:266), DOM11-44 (SEQ ID NO:267), DOM11-45 (SEQ ID NO:268), DOM11-5 (SEQ ID NO:236), DOM11-7 (SEQ ID NO:238), DOM11-1 (SEQ ID NO:232), DOM11-10 (SEQ ID NO:241), DOM11-16 (SEQ ID NO:243), DOM11-2 (SEQ ID NO:233), DOM11-20 (SEQ ID NO:244), DOM11-21 (SEQ ID NO:245), DOM11-24 (SEQ ID NO:248), DOM11-28 (SEQ ID NO:252), DOM11-33 (SEQ ID NO:257), DOM11-34 (SEQ ID NO:258), DOM11-35 (SEQ ID NO:259), DOM11-37 (SEQ ID NO:261), DOM11-38 (SEQ ID NO:262), DOM11-39 (SEQ ID NO:293), DOM11-41 (SEQ ID NO:264), DOM11-42 (SEQ ID NO:265), DOM11-6 (SEQ ID NO:237), DOM11-8 (SEQ ID NO:239), and DOM11-9 (SEQ ID NO:240).

In other embodiments, the first immunoglobulin single variable domain or the second immunoglobulin single variable domain comprises an amino acid sequence that has at least about 90% amino acid sequence similarity with the amino acid sequence of a dAb selected from the group consisting of: DOM 11-3-1 (SEQ ID NO: 269), DOM 11-3-2 (SEQ ID NO: 270), DOM 11-3-3 (SEQ ID NO: 271), DOM 11-3-4 (SEQ ID NO: 272), DOM 11-3-6 (SEQ ID NO: 273), DOM 11-3-9 (SEQ ID NO: 274), DOM 11-3-10 (SEQ ID NO: 275), DOM 11-3-11 (SEQ ID NO: 276), DOM 11-3-14 (SEQ ID NO: 277), DOM 11-3-15 (SEQ ID NO: 278), DOM 11-3-17 (SEQ ID NO: 279), DOM 11-3-19 (SEQ ID NO: 280), DOM 11-3-20 (SEQ ID NO: 281), DOM 11-3-21 (SEQ ID NO: 282), DOM 11-3-22 (SEQ ID NO: 283), DOM 11-3-23 (SEQ ID NO: 284), DOM 11-3-24 (SEQ ID NO: 285), DOM 11-3-25 (SEQ ID NO: 286), DOM 11-3-26 (SEQ ID NO: 287), DOM 11-3-27 (SEQ ID NO: 288), DOM 11-3-28 (SEQ ID NO: 289), DOM 11-30-1 (SEQ ID NO: 290), DOM 11-30-2 (SEQ ID NO: 291), DOM 11-30-3 (SEQ ID NO: 292), DOM 11-30-5 (SEQ ID NO: 293), DOM 11-30-6 (SEQ ID NO: 294), DOM 11-30-7 (SEQ ID NO: 295), DOM 11-30-8 (SEQ ID NO: 296), DOM 11-30-9 (SEQ ID NO: 297), DOM 11-30-10 (SEQ ID NO: 298), DOM 11-30-11 (SEQ ID NO: 299), DOM 11-30-12 (SEQ ID NO: 300), DOM 11-30-13 (SEQ ID NO: 301), DOM 11-30-14 (SEQ ID NO: 302), DOM 11-30-15 (SEQ ID NO: 303), DOM 11-30-16 (SEQ ID NO: 304), and DOM 11-30-17 (SEQ ID NO: 305).

In other embodiments, the first immunoglobulin single variable domain or the second immunoglobulin single variable domain binds CD138 and competes for binding to CD138 with an anti-CD138 domain antibody (dAb) selected from the group consisting of: DOM12-1 (SEQ ID NO:289), DOM12-15 (SEQ ID NO:290), DOM12-17 (SEQ ID NO:11), DOM12-19 (SEQ ID NO:291), DOM12-2 (SEQ ID NO:292), DOM12-20 (SEQ ID NO:293), DOM12-21 (SEQ ID NO:294), DOM12-22 (SEQ ID NO:295), DOM12-3 (SEQ ID NO:296), DOM12-33 (SEQ ID NO:297), DOM12-39 (SEQ ID NO:298), DOM12-4 (SEQ ID NO:299), DOM12-40 (SEQ ID NO:300), DOM12-41 (SEQ ID NO:301), DOM12-42 (SEQ ID NO:302), DOM12-44 (SEQ ID NO:303), DOM12-46 (SEQ ID NO:304), DOM12-6 (SEQ ID NO:305), DOM12-7 (SEQ ID NO:306), DOM12-10 (SEQ ID NO:307), DOM12-11 (SEQ ID NO:308), DOM12-18 (SEQ ID NO:309), DOM12-23 (SEQ ID NO:310), DOM12-24 (SEQ ID NO:311), DOM12-25 (SEQ ID NO:312), DOM12-26 (SEQ ID NO:12), DOM12-27 (SEQ ID NO:313), DOM12-28 (SEQ ID NO:314), DOM12-29 (SEQ ID NO:315), DOM12-30 (SEQ ID NO:316), DOM12-31 (SEQ ID NO:317), DOM12-32 (SEQ ID NO:318), DOM12-34 (SEQ ID NO:319), DOM12-35 (SEQ ID NO:320), DOM12-36 (SEQ ID NO:321), DOM12-37 (SEQ ID NO:322), DOM12-38 (SEQ ID NO:323), DOM12-43 (SEQ ID NO:324), DOM12-45 (SEQ ID NO:310), DOM12-5 (SEQ ID NO:325), DOM12-8 (SEQ ID NO:326), and DOM12-9 (SEQ ID NO:327).

In certain embodiments, the first immunoglobulin single variable domain or the second immunoglobulin single variable domain binds CD138 and competes for binding to CD138 with an anti-CD138 domain antibody (dAb) selected from the group consisting of: DOM 12-45-1 (SEQ ID NO: 348), DOM 12-45-2 (SEQ ID NO: 349), DOM 12-45-3 (SEQ ID NO: 350), DOM 12-45-4 (SEQ ID NO: 351), DOM 12-45-5 (SEQ ID NO: 352), DOM 12-45-6 (SEQ ID NO: 353), DOM 12-45-8 (SEQ ID NO: 354), DOM 12-45-9 (SEQ ID NO: 355), DOM 12-45-10 (SEQ ID NO: 356), DOM 12-45-11 (SEQ ID NO: 357), DOM 12-45-12 (SEQ ID NO: 358), DOM 12-45-13 (SEQ ID NO: 359), DOM 12-45-14 (SEQ ID NO: 360), DOM 12-45-15 (SEQ ID NO: 361), DOM 12-45-16 (SEQ ID NO: 362), DOM 12-45-17 (SEQ ID NO: 363), DOM 12-45-18 (SEQ ID NO: 364), DOM 12-45-19 (SEQ ID NO: 365), DOM 12-45-20 (SEQ ID NO: 366), DOM 12-45-21 (SEQ ID NO: 367), DOM 12-45-22 (SEQ ID NO: 368), DOM 12-45-23 (SEQ ID NO: 369), DOM 12-45-24 (SEQ ID NO: 370), DOM 12-45-25 (SEQ ID NO: 371), DOM 12-45-26 (SEQ ID NO: 372), DOM 12-45-27 (SEQ ID NO: 373), DOM 12-45-28 (SEQ ID NO: 374), DOM 12-45-29 (SEQ ID NO: 375), DOM 12-45-30 (SEQ ID NO: 376), DOM 12-45-31 (SEQ ID NO: 377), DOM 12-45-32 (SEQ ID NO: 378), DOM 12-45-33 (SEQ ID NO: 379), DOM 12-45-34 (SEQ ID NO: 380), DOM 12-45-35 (SEQ ID NO: 381), DOM 12-45-36 (SEQ ID NO: 382), DOM 12-45-37 (SEQ ID NO: 383), and DOM 12-45-38 (SEQ ID NO: 384).

In other embodiments, the first immunoglobulin single variable domain or the second immunoglobulin single variable domain comprises an amino acid sequence that has at least about 90% amino acid sequence similarity with the amino acid sequence of a dAb selected from the group consisting of: DOM12-1 (SEQ ID NO:289), DOM12-15 (SEQ ID NO:290), DOM12-17 (SEQ ID NO:11), DOM12-19 (SEQ ID NO:291), DOM12-2 (SEQ ID NO:292), DOM12-20 (SEQ ID NO:293), DOM12-21 (SEQ ID NO:294), DOM12-22 (SEQ ID NO:295), DOM12-3 (SEQ ID NO:296), DOM12-33 (SEQ ID NO:297), DOM12-39 (SEQ ID NO:298), DOM12-4 (SEQ ID NO:299), DOM12-40 (SEQ ID NO:300), DOM12-41 (SEQ ID NO:301), DOM12-42 (SEQ ID NO:302), DOM12-44 (SEQ ID NO:303), DOM12-46 (SEQ ID NO:304), DOM12-6 (SEQ ID NO:305), DOM12-7 (SEQ ID NO:306), DOM12-10 (SEQ ID NO:307), DOM12-11 (SEQ ID NO:308), DOM12-18 (SEQ ID NO:309), DOM12-23 (SEQ ID NO:310), DOM12-24 (SEQ ID NO:313), DOM12-25 (SEQ ID NO:312), DOM12-26 (SEQ ID NO:12), DOM12-27 (SEQ ID NO:313), DOM12-28 (SEQ ID NO:314), DOM12-29 (SEQ ID NO:315), DOM12-30 (SEQ ID NO:316), DOM12-31 (SEQ ID NO:317), DOM12-32 (SEQ ID NO:318), DOM12-34 (SEQ ID NO:319), DOM12-35 (SEQ ID NO:320), DOM12-36 (SEQ ID NO:321), DOM12-37 (SEQ ID NO:322), DOM12-38 (SEQ ID NO:323), DOM12-43 (SEQ ID NO:324), DOM12-45 (SEQ ID NO:310), DOM12-5 (SEQ ID NO:325), DOM12-8 (SEQ ID NO:326), and DOM12-9 (SEQ ID NO:327).

In certain embodiments, the first immunoglobulin single variable domain or the second immunoglobulin single variable domain comprises an amino acid sequence that has at least about 90% amino acid sequence similarity with the amino acid sequence of a dAb selected from the group consisting of: DOM 12-45-1 (SEQ ID NO: 348), DOM 12-45-2 (SEQ ID NO: 349), DOM 12-45-3 (SEQ ID NO: 350), DOM 12-45-4 (SEQ ID NO: 351), DOM 12-45-5 (SEQ ID NO: 352), DOM 12-45-6 (SEQ ID NO: 353), DOM 12-45-8 (SEQ ID NO: 354), DOM 12-45-9 (SEQ ID NO: 355), DOM 12-45-10 (SEQ ID NO: 356), DOM 12-45-11 (SEQ ID NO: 357), DOM 12-45-12 (SEQ ID NO: 358), DOM 12-45-13 (SEQ ID NO: 359), DOM 12-45-14 (SEQ ID NO: 360), DOM 12-45-15 (SEQ ID NO: 361), DOM 12-45-16 (SEQ ID NO: 362), DOM 12-45-17 (SEQ ID NO: 363), DOM 12-45-18 (SEQ ID NO: 364), DOM 12-45-19 (SEQ ID NO: 365), DOM 12-45-20 (SEQ ID NO: 366), DOM 12-45-21 (SEQ ID NO: 367), DOM 12-45-22 (SEQ ID NO: 368), DOM 12-45-23 (SEQ ID NO: 369), DOM 12-45-24 (SEQ ID NO: 370), DOM 12-45-25 (SEQ ID NO: 371), DOM 12-45-26 (SEQ ID NO: 372), DOM 12-45-27 (SEQ ID NO: 373), DOM 12-45-28 (SEQ ID NO: 374), DOM 12-45-29 (SEQ ID NO: 375), DOM 12-45-30 (SEQ ID NO: 376), DOM 12-45-31 (SEQ ID NO: 377), DOM 12-45-32 (SEQ ID NO: 378), DOM 12-45-33 (SEQ ID NO: 379), DOM 12-45-34 (SEQ ID NO: 380), DOM 12-45-35 (SEQ ID NO: 381), DOM 12-45-36 (SEQ ID NO: 382), DOM 12-45-37 (SEQ ID NO: 383), and DOM 12-45-38 (SEQ ID NO: 384).

In other embodiments, the first immunoglobulin single variable domain or the second immunoglobulin single variable domain binds CEA and competes for binding to CEA with an anti-CEA domain antibody (dAb) selected from the group consisting of: DOM13-1 (SEQ ID NO:328), DOM13-12 (SEQ ID NO:329), DOM13-13 (SEQ ID NO:330), DOM13-14 (SEQ ID NO:331), DOM3-15 (SEQ ID NO:332), DOM13-16 (SEQ ID NO:333), DOM13-17 (SEQ ID NO:334), DOM13-18 (SEQ ID NO:335), DOM13-19 (SEQ ID NO:336), DOM13-2 (SEQ ID NO:337), DOM13-20 (SEQ ID NO:338), DOM13-21 (SEQ ID NO:339), DOM13-22 (SEQ ID NO:340), DOM13-23 (SEQ ID NO:341), DOM13-24 (SEQ ID NO:342), DOM13-25 (SEQ ID NO:313), DOM13-26 (SEQ ID NO:343), DOM13-27 (SEQ ID NO:344), DOM13-28 (SEQ ID NO:345), DOM13-29 (SEQ ID NO:346), DOM13-3 (SEQ ID NO:347), DOM13-30 (SEQ ID NO:348), DOM13-31 (SEQ ID NO:349), DOM13-32 (SEQ ID NO:350), DOM13-33 (SEQ ID NO:351), DOM-13-34 (SEQ ID NO:352), DOM13-35 (SEQ ID NO:353), DOM13-36 (SEQ ID NO:354), DOM13-37 (SEQ ID NO:355), DOM13-4 (SEQ ID NO:356), DOM13-42 (SEQ ID NO:357), DOM13-43 (SEQ ID NO:358), DOM13-44 (SEQ ID NO:359), DOM13-45 (SEQ ID NO:360), DOM13-46 (SEQ ID NO:361), DOM13-47 (SEQ ID NO:362), DOM13-48 (SEQ ID NO:363), DOM13-49 (SEQ ID NO:364), DOM13-5 (SEQ ID NO:365), DOM13-50 (SEQ ID NO:366), DOM13-51 (SEQ ID NO:367), DOM13-52 (SEQ ID NO:368), DOM13-53 (SEQ ID NO:369), DOM13-54 (SEQ ID NO:370), DOM13-55 (SEQ ID NO:371), DOM13-56 (SEQ ID NO:372), DOM13-57 (SEQ ID NO:14), DOM13-58 (SEQ ID NO:15), DOM13-59 (SEQ ID NO:16), DOM13-6 (SEQ ID NO:373), DOM13-60 (SEQ ID NO:374), DOM13-61 (SEQ ID NO:375), DOM13-62 (SEQ ID NO:376), DOM13-63 (SEQ ID NO:377), DOM13-64 (SEQ ID NO:17), DOM13-65 (SEQ ID NO:18), DOM13-66 (SEQ ID NO:378), DOM13-67 (SEQ ID NO:379), DOM13-68 (SEQ ID NO:380), DOM13-69 (SEQ ID NO:381), DOM13-7 (SEQ ID NO:382), DOM13-70 (SEQ ID NO:383), DOM13-71 (SEQ ID NO:384), DOM13-72 (SEQ ID NO:385), DOM13-73 (SEQ ID NO:386), DOM13-74 (SEQ ID NO:19), DOM13-75 (SEQ ID NO:387), DOM13-76 (SEQ ID NO:388), DOM13-77 (SEQ ID NO:389), DOM13-78 (SEQ ID NO:390), DOM13-79 (SEQ ID NO:391), DOM13-8 (SEQ ID NO:392), DOM13-80 (SEQ ID NO:393), DOM13-81 (SEQ ID NO:394), DOM13-82 (SEQ ID NO:395), DOM13-83 (SEQ ID NO:396), DOM13-84 (SEQ ID NO:397), DOM13-85 (SEQ ID NO:398), DOM13-86 (SEQ ID NO:399), DOM13-87 (SEQ ID NO:400), DOM13-88 (SEQ ID NO:401), DOM13-89 (SEQ ID NO:402), DOM13-90 (SEQ ID NO:403), DOM13-91 (SEQ ID NO:404), DOM13-92 (SEQ ID NO:405), DOM13-93 (SEQ ID NO:20), DOM13-94 (SEQ ID NO:406), and DOM13-95 (SEQ ID NO:21).

In certain embodiments, the first immunoglobulin single variable domain or the second immunoglobulin single variable domain binds CEA and competes for binding to CEA with an anti-CEA domain antibody (dAb) selected from the group consisting of: DOM 13-25-3 (SEQ ID NO: 473), DOM 13-25-23 (SEQ ID NO: 474), DOM 13-25-27 (SEQ ID NO: 475), and DOM 13-25-80 (SEQ ID NO: 476).

In other embodiments, the first immunoglobulin single variable domain or the second immunoglobulin single variable domain comprises an amino acid sequence that has at least about 90% amino acid sequence similarity with the amino acid sequence of a dAb selected from the group consisting of: DOM13-1 (SEQ ID NO:328), DOM13-12 (SEQ ID NO:329), DOM13-13 (SEQ ID NO:330), DOM13-14 (SEQ ID NO:331), DOM13-15 (SEQ ID NO:332), DOM13-16 (SEQ ID NO:333), DOM13-17 (SEQ ID NO:334), DOM13-18 (SEQ ID NO:335), DOM13-19 (SEQ ID NO:336), DOM13-2 (SEQ ID NO:337), DOM13-20 (SEQ ID NO:338), DOM13-21 (SEQ ID NO:339), DOM13-22 (SEQ ID NO:340), DOM13-23 (SEQ ID NO:341), DOM13-24 (SEQ ID NO:342), DOM13-25 (SEQ ID NO:13), DOM13-26 (SEQ ID NO:343), DOM13-27 (SEQ ID NO:344), DOM13-28 (SEQ ID NO:345), DOM13-29 (SEQ ID NO:346), DOM13-3 (SEQ ID NO:347), DOM13-30 (SEQ ID NO:348), DOM13-31 (SEQ ID NO:349), DOM13-32 (SEQ ID NO:350), DOM13-33 (SEQ ID NO:351), DOM-13-34 (SEQ ID NO:352), DOM13-35 (SEQ ID NO:353), DOM13-36 (SEQ ID NO:354), DOM13-37 (SEQ ID NO:355), DOM13-4 (SEQ ID NO:356), DOM13-42 (SEQ ID NO:357), DOM13-43 (SEQ ID NO:358), DOM13-44 (SEQ ID NO:359), DOM13-45 (SEQ ID NO:360), DOM13-46 (SEQ ID NO:361), DOM13-47 (SEQ ID NO:362), DOM13-48 (SEQ ID NO:363), DOM13-49 (SEQ ID NO:364), DOM13-5 (SEQ ID NO:365), DOM13-50 (SEQ ID NO:366), DOM13-51 (SEQ ID NO:367), DOM13-52 (SEQ ID NO:368), DOM13-53 (SEQ ID NO:369), DOM13-54 (SEQ ID NO:370), DOM13-55 (SEQ ID NO:371), DOM13-56 (SEQ ID NO:372), DOM13-57 (SEQ ID NO:14), DOM13-58 (SEQ ID NO:15), DOM13-59 (SEQ ID NO:16), DOM13-6 (SEQ ID NO:373), DOM13-60 (SEQ ID NO:374), DOM13-61 (SEQ ID NO:375), DOM13-62 (SEQ ID NO:376), DOM13-63 (SEQ ID NO:377), DOM13-64 (SEQ ID NO:17), DOM13-65 (SEQ ID NO:18), DOM13-66 (SEQ ID NO:378), DOM13-67 (SEQ ID NO:379), DOM13-68 (SEQ ID NO:380), DOM13-69 (SEQ ID NO:381), DOM13-7 (SEQ ID NO:382), DOM13-70 (SEQ ID NO:383), DOM13-71 (SEQ ID NO:384), DOM13-72 (SEQ ID NO:385), DOM13-73 (SEQ ID NO: 386), DOM13-74 (SEQ ID NO:19), DOM 13-75 (SEQ ID NO:387), DOM13-76 (SEQ ID NO:388), DOM13-77 (SEQ ID NO:389), DOM13-78 (SEQ ID NO:390), DOM13-79 (SEQ ID NO:391), DOM13-8 (SEQ ID NO:392), DOM13-80 (SEQ ID NO:393), DOM13-81 (SEQ ID NO:394), DOM13-82 (SEQ ID NO:395), DOM13-83 (SEQ ID NO:396), DOM13-84 (SEQ ID NO:397), DOM13-85 (SEQ ID NO:398), DOM13-86 (SEQ ID NO:399), DOM13-87 (SEQ ID NO:400), DOM13-88 (SEQ ID NO:401), DOM13-89 (SEQ ID NO:402), DOM13-90 (SEQ ID NO:403), DOM13-91 (SEQ ID NO:404), DOM13-92 (SEQ ID NO:405), DOM13-93 (SEQ ID NO:20), DOM13-94 (SEQ ID NO:406), and DOM13-95 (SEQ ID NO:21).

In certain embodiments, the first immunoglobulin single variable domain or the second immunoglobulin single variable domain comprises an amino acid sequence that has at least about 90% amino acid sequence similarity with the amino acid sequence of a dAb selected from the group consisting of: DOM 13-25-3 (SEQ ID NO: 473), DOM 13-25-23 (SEQ ID NO: 474), DOM 13-25-27 (SEQ ID NO: 475), and DOM 13-25-80 (SEQ ID NO: 476).

In other embodiments, the first immunoglobulin single variable domain or the second immunoglobulin single variable domain binds CD56 and competes for binding to CD56 with an anti-CD56 domain antibody (dAb) selected from the group consisting of: DOM14-1 (SEQ ID NO:477), DOM14-10 (SEQ ID NO:481), DOM14-100 (SEQ ID NO:540), DOM14-11 (SEQ ID NO:482), DOM14-12 (SEQ ID NO:483), DOM14-13 (SEQ ID NO:484), DOM14-14 (SEQ ID NO:485), DOM14-15 (SEQ ID NO:486), DOM14-16 (SEQ ID NO:487), DOM14-17 (SEQ ID NO:488), DOM14-18 (SEQ ID NO:489), DOM14-19 (SEQ ID NO:490), DOM14-2 (SEQ ID NO:478), DOM14-20 (SEQ ID NO:491), DOM14-21 (SEQ ID NO:492), DOM14-22 (SEQ ID NO:493), DOM14-23 (SEQ ID NO:494), DOM14-24 (SEQ ID NO:495), DOM14-25 (SEQ ID NO:496), DOM14-26 (SEQ ID NO:497), DOM14-27 (SEQ ID NO:498), DOM14-28 (SEQ ID NO:499), DOM14-3 (SEQ ID NO:479), DOM14-31 (SEQ ID NO:500), DOM14-32 (SEQ ID NO:501), DOM14-33 (SEQ ID NO:502), DOM14-34 (SEQ ID NO:503), DOM14-35 (SEQ ID NO:504), DOM14-36 (SEQ ID NO:505), DOM14-37 (SEQ ID NO:506), DOM14-38 (SEQ ID NO:507), DOM14-39 (SEQ ID NO:508), DOM14-4 (SEQ ID NO:480), DOM14-40 (SEQ ID NO:509), DOM14-41 (SEQ ID NO:510), DOM14-42 (SEQ ID NO:511), DOM14-43 (SEQ ID NO:512), DOM14-44 (SEQ ID NO:513), DOM14-45 (SEQ ID NO:514), DOM14-46 (SEQ ID NO:515), DOM14-47 (SEQ ID NO:516), DOM14-48 (SEQ ID NO:517), DOM14-49 (SEQ ID NO:518), DOM14-50 (SEQ ID NO:519), DOM14-51 (SEQ ID NO:520), DOM14-52 (SEQ ID NO:521), DOM14-53 (SEQ ID NO:522), DOM14-54 (SEQ ID NO:523), DOM14-55 (SEQ ID NO:524), DOM14-56 (SEQ ID NO:525), DOM14-57 (SEQ ID NO:526), DOM14-58 (SEQ ID NO:527), DOM14-59 (SEQ ID NO:528), DOM14-60 (SEQ ID NO:529), DOM14-61 (SEQ ID NO:530), DOM14-62 (SEQ ID NO:531), DOM14-63 (SEQ ID NO:532), DOM14-64 (SEQ ID NO:533), DOM14-65 (SEQ ID NO:534), DOM14-66 (SEQ ID NO:535), DOM14-67 (SEQ ID NO:536), DOM14-70 (SEQ ID NO:539), DOM14-68 (SEQ ID NO:537), and DOM14-69 (SEQ ID NO:538).

In other embodiments, the first immunoglobulin single variable domain or the second immunoglobulin single variable domain comprises an amino acid sequence that has at least about 90% amino acid sequence similarity with the amino acid sequence of a dAb selected from the group consisting of: DOM14-1 (SEQ ID NO:477), DOM14-10 (SEQ ID NO:481), DOM14-100 (SEQ ID NO:540), DOM14-11 (SEQ ID NO:482), DOM14-12 (SEQ ID NO:483), DOM14-13 (SEQ ID NO:484), DOM14-14 (SEQ ID NO:485), DOM14-15 (SEQ ID NO:486), DOM14-16 (SEQ ID NO:487), DOM14-17 (SEQ ID NO:488), DOM14-18 (SEQ ID NO:489), DOM14-19 (SEQ ID NO:490), DOM14-2 (SEQ ID NO:478), DOM14-20 (SEQ ID NO:491), DOM14-21 (SEQ ID NO:492), DOM14-22 (SEQ ID NO:493), DOM14-23 (SEQ ID NO:494), DOM14-24 (SEQ ID NO:495), DOM14-25 (SEQ ID NO:496), DOM14-26 (SEQ ID NO:497), DOM14-27 (SEQ ID NO:498), DOM14-28 (SEQ ID NO:499), DOM14-3 (SEQ ID NO:479), DOM14-31 (SEQ ID NO:500), DOM14-32 (SEQ ID NO:501), DOM14-33 (SEQ ID NO:502), DOM14-34 (SEQ ID NO:503), DOM14-35 (SEQ ID NO:504), DOM14-36 (SEQ ID NO:505), DOM14-37 (SEQ ID NO:506), DOM14-38 (SEQ ID NO:507), DOM14-39 (SEQ ID NO:508), DOM14-4 (SEQ ID NO:480), DOM14-40 (SEQ ID NO:509), DOM14-41 (SEQ ID NO:510), DOM14-42 (SEQ ID NO:511), DOM14-43 (SEQ ID NO:512), DOM14-44 (SEQ ID NO:513), DOM14-45 (SEQ ID NO:514), DOM14-46 (SEQ ID NO:515), DOM14-47 (SEQ ID NO:516), DOM14-48 (SEQ ID NO:517), DOM14-49 (SEQ ID NO:518), DOM14-50 (SEQ ID NO:519), DOM14-51 (SEQ ID NO:520), DOM14-52 (SEQ ID NO:521), DOM14-53 (SEQ ID NO:522), DOM14-54 (SEQ ID NO:523), DOM14-55 (SEQ ID NO:524), DOM14-56 (SEQ ID NO:525), DOM14-57 (SEQ ID NO:526), DOM14-58 (SEQ ID NO:527), DOM14-59 (SEQ ID NO:528), DOM14-60 (SEQ ID NO:529), DOM14-61 (SEQ ID NO:530), DOM14-62 (SEQ ID NO:531), DOM14-63 (SEQ ID NO:532), DOM14-64 (SEQ ID NO:533), DOM14-65 (SEQ ID NO:534), DOM14-66 (SEQ ID NO:535), DOM14-67 (SEQ ID NO:536), DOM14-70 (SEQ ID NO:539), DOM14-68 (SEQ ID NO:537), and DOM14-69 (SEQ ID NO:538).

In more particular embodiments, the first immunoglobulin single variable domain has a binding site with binding specificity CD38, and the second immunoglobulin single variable domain has a binding site with binding specificity for a cell surface target selected from the group consisting of CD138, CEA, CD56, VEGF, EGFR, and HER2. In certain embodiments, the second immunoglobulin single variable domain has a binding site with binding specificity for CD138.

In another embodiment, the first immunoglobulin single variable domain has a binding site with binding specificity CD138, and the second immunoglobulin single variable domain has a binding site with binding specificity for a cell surface target selected from the group consisting of CD38, CEA, CD56, VEGF, EGFR, and HER2. In certain embodiments, the second immunoglobulin single variable domain has a binding site with binding specificity for CEA.

In other embodiments, the first immunoglobulin single variable domain has a binding site with binding specificity CEA, and the second immunoglobulin single variable domain has a binding site with binding specificity for a cell surface target selected from the group consisting of CD38, CD38, CEA, VEGF, EGFR, and HER2. In certain embodiments, the second immunoglobulin single variable domain has a binding site with binding specificity for CD56.

If desired, the ligand can further comprise a toxin, such as a surface active toxin. The surface active toxin can comprise a free radical generator or a radionuclide.

In some embodiments, the ligand further comprises a half-life extending moiety, such as a polyalkylene glycol moiety, serum albumin or a fragment thereof, transferrin receptor or a transferrin-binding portion thereof, or an antibody or antibody fragment comprising a binding site for a polypeptide that enhances half-life in vivo. In some embodiments, the half-life extending moiety is a polyethylene glycol moiety.

In other embodiments, the half-life extending moiety is an antibody or antibody fragment, such as an immunoglobulin single variable domain, comprising a binding site for serum albumin or neonatal Fc receptor.

In particular embodiments, the half-life extending moiety is an immunoglobulin single variable domain that competes for binding to human serum albumin with a dAb selected from the group consisting of: DOM7m-16 (SEQ ID NO: 541), DOM7m-12 (SEQ ID NO: 542), DOM7m-26 (SEQ ID NO: 543), DOM7r-1 (SEQ ID NO: 544), DOM7r-3 (SEQ ID NO: 545), DOM7r-4 (SEQ ID NO: 546), DOM7r-5 (SEQ ID NO: 547), DOM7r-7 (SEQ ID NO: 548), and DOM7r-8 (SEQ ID NO: 549), DOM7h-2 (SEQ ID NO: 550), DOM7h-3 (SEQ ID NO: 551), DOM7h-4 (SEQ ID NO: 552), DOM7h-6 (SEQ ID NO: 553), DOM7h-1 (SEQ ID NO: 555), DOM7h-7 (SEQ ID NO: 477), DOM7h-8 (SEQ ID NO: 564), DOM7r-13 (SEQ ID NO: 565), and DOM7r-14 (SEQ ID NO: 566), DOM7h-22 (SEQ ID NO: 557), DOM7h-23 (SEQ ID NO: 558), DOM7h-24 (SEQ ID NO: 559), DOM7h-25 (SEQ ID NO: 560), DOM7h-26 (SEQ ID NO: 561), DOM7h-21 (SEQ ID NO: 562), DOM7h-27 (SEQ ID NO: 563), DOM7r-15 (SEQ ID NO: 567), DOM7r-16 (SEQ ID NO: 568), DOM7r-17 (SEQ ID NO: 569), DOM7r-18 (SEQ ID NO: 570), DOM7r-19 (SEQ ID NO: 571), DOM7r-20 (SEQ ID NO: 572), DOM7r-21 (SEQ ID NO: 573), DOM7r-22 (SEQ ID NO: 574), DOM7r-23 (SEQ ID NO: 575), DOM7r-24 (SEQ ID NO: 576), DOM7r-25 (SEQ ID NO: 577), DOM7r-26 (SEQ ID NO: 578), DOM7r-27 (SEQ ID NO: 579), DOM7r-28 (SEQ ID NO: 580), DOM7r-29 (SEQ ID NO: 581), DOM7r-30 (SEQ ID NO: 582), DOM7r-31 (SEQ ID NO: 583), DOM7r-32 (SEQ ID NO: 584), and DOM7r-33 (SEQ ID NO: 585).

In another embodiment, the half-life extending moiety is an immunoglobulin single variable domain that binds human serum albumin and comprises an amino acid sequence that has at least 90% amino acid sequence identity with the amino acid sequence of a dAb selected from the group consisting of: DOM7m-16 (SEQ ID NO: 541), DOM7m-12 (SEQ ID NO: 542), DOM7m-26 (SEQ ID NO: 543), DOM7r-1 (SEQ ID NO: 544), DOM7r-3 (SEQ ID NO: 545), DOM7r-4 (SEQ ID NO: 546), DOM7r-5 (SEQ ID NO: 547), DOM7r-7 (SEQ ID NO: 548), and DOM7r-8 (SEQ ID NO: 549), DOM7h-2 (SEQ ID NO: 550), DOM7h-3 (SEQ ID NO: 551), DOM7h-4 (SEQ ID NO: 552), DOM7h-6 (SEQ ID NO: 553), DOM7h-1 (SEQ ID NO: 555), DOM7h-7 (SEQ ID NO: 477), DOM7h-8 (SEQ ID NO: 564), DOM7r-13 (SEQ ID NO: 565), and DOM7r-14 (SEQ ID NO: 566), DOM7h-22 (SEQ ID NO: 557), DOM7h-23 (SEQ ID NO: 558), DOM7h-24 (SEQ ID NO: 559), DOM7h-25 (SEQ ID NO: 560), DOM7h-26 (SEQ ID NO: 561), DOM7h-21 (SEQ ID NO: 562), DOM7h-27 (SEQ ID NO: 563), DOM7r-15 (SEQ ID NO: 567), DOM7r-16 (SEQ ID NO: 568), DOM7r-17 (SEQ ID NO: 569), DOM7r-18 (SEQ ID NO: 570), DOM7r-19 (SEQ ID NO: 571), DOM7r-20 (SEQ ID NO: 572), DOM7r-21 (SEQ ID NO: 573), DOM7r-22 (SEQ ID NO: 574), DOM7r-23 (SEQ ID NO: 575), DOM7r-24 (SEQ ID NO: 576), DOM7r-25 (SEQ ID NO: 577), DOM7r-26 (SEQ ID NO: 578), DOM7r-27 (SEQ ID NO: 579), DOM7r-28 (SEQ ID NO: 580), DOM7r-29 (SEQ ID NO: 581), DOM7r-30 (SEQ ID NO: 582), DOM7r-31 (SEQ ID NO: 583), DOM7r-32 (SEQ ID NO: 584), and DOM7r-33 (SEQ ID NO: 585).

In another aspect, the ligand comprises a first polypeptide domain having a binding site with binding specificity for a first cell surface target, a second polypeptide domain having a binding site with binding specificity for a second cell surface target, and at least one toxin moiety; wherein said first cell surface target and said second cell surface target are different, and said first cell surface target and said second cell surface target are present on a pathogenic cell; wherein said ligand binds said first cell surface target and said second cell surface target on said pathogenic cell with an avidity between about 10⁻⁶ M and about 10⁻¹² M; and wherein said ligand is internalized by said pathogenic cell. As described herein, the toxin can be a surface active toxin. The surface active toxin can comprise a free radical generator or a radionuclide.

Preferably, the ligand is preferentially internalized by a pathogenic cell. For example, the ligand is not substantially internalized by single positive or normal cells, or selectively binds a pathogenic cell. In some embodiments, the ligand selectively binds a pathogenic cell when said ligand is present at a concentration that is between about 1 pM and about 150 nM.

The invention also relates to a ligand for use in therapy or diagnosis, and to the use of a ligand for the manufacture of a medicament for treating a disease as described herein (e.g., cancer, multiple myeloma, lung carcinoma).

The invention also relates to the use of a ligand for the manufacture of a medicament for selectively killing cancer cells over normal cells.

The invention also relates to the use of a ligand for the manufacture of a medicament for delivering a therapeutic agent intracellularly.

The invention also relates to the use of a ligand for the manufacture of a medicament for delivering a therapeutic agent to a cathepsin B compartment in a cell.

The invention also relates to the use of a ligand for the manufacture of a medicament for localizing the ligand to a cathepsin B compartment in a cell.

The invention also relates to a method for treating a disease comprising administering to a subject in need thereof a therapeutically effective amount of a ligand of the invention. In some embodiments, the disease is cancer, for example, multiple myeloma or lung cancer (e.g., small cell lung carcinoma).

The invention also relates to a method of delivering a therapeutic agent (e.g., a toxin) internally to a cell, comprising contacting a cell with a ligand of the invention.

The invention also relates to a composition (e.g., a pharmaceutical composition) comprising a ligand of the invention and a physiologically acceptable carrier. In some embodiments, the composition comprises a vehicle for intravenous, intramuscular, intraperitoneal, intraarterial, intrathecal, intraarticular, or subcutaneous administration. In other embodiments, the composition comprises a vehicle for pulmonary, intranasal, vaginal, or rectal administration.

The invention also relates to a drug delivery device comprising the composition of the invention. In some embodiments, the drug delivery device is selected from the group consisting of a parenteral delivery device, intravenous delivery device, intramuscular delivery device, intraperitoneal delivery device, transdermal delivery device, pulmonary delivery device, intraarterial delivery device, intrathecal delivery device, intraarticular delivery device, subcutaneous delivery device, intranasal delivery device, vaginal delivery device, and rectal delivery device. In other embodiments, the drug delivery device is selected from the group consisting of a syringe, a transdermal delivery device, a capsule, a tablet, a nebulizer, an inhaler, an atomizer, an aerosolizer, a mister, a dry powder inhaler, a metered dose inhaler, a metered dose sprayer, a metered dose mister, a metered dose atomizer and a catheter.

The invention also relates to an isolated or recombinant nucleic acid encoding a ligand the invention, and to a vector comprising the recombinant nucleic acid of the invention and to a host cell comprising the recombinant nucleic acid or the vector of the invention. The invention also relates to a method for producing a ligand comprising maintaining a host cell of the invention under conditions suitable for expression of the nucleic acid or vector of the invention, whereby a ligand is produced. In some embodiments, the method further comprises isolating the ligand.

In some embodiments, the ligand of the invention is internalized by cells that contain the cell surface targets. For example, at least about 40%, or at least about 50%, or at least about 60%, or at least about 70%, or at least about 80%, or at least about 90% or substantially all of the ligand is internalized by a cell (e.g., the ligand that binds a double positive cell or pathogenic cell).

The invention also relates to the domain antibodies disclosed herein, and to ligands and formats comprising same. The invention also relates to isolated or recombinant nucleic acids encoding the domain antibodies disclosed herein, and to vectors that comprise the recombinant nucleic acid, and to host cells that comprise the recombinant nucleic acid or vector. The invention also relates to a method for producing a dAb disclosed herein, or a ligand or format comprising such a dAb, comprising maintaining a host cell of the invention under conditions suitable for expression of the nucleic acid or vector of the invention, whereby a dAb disclosed herein, or ligand or format comprising such a dAb is produced. In some embodiments, the method further comprises isolating the ligand.

### BRIEF DESCRIPTION OF THE DRAWINGS

FIGS. 1A-1H are fluorescence histograms showing binding specificity of dAbs that bind CD38, CD138, CEA or CD56. FIGS. 1A and 1B show that dAbs that bind CD38 (DOM11-3 and DOM11-30) bind to CD38+ cells (RPMI cells) but not to CD38− cells (K299 cells). FIGS. 1C and 1D show a dAb that binds CD138 (DOM12-45) binds to CD138+ cells (RPMI cells) but not to CD138− cells (K299 cells). FIGS. 1E and 1F show that a dAb that binds CEA (DOM13-25) binds to CEA+ cells (H69 cells) but not to CEA− cells (CHO cells). FIGS. 1G and 1H show that a dAb that binds CD56 (DOM14-23) binds to CD56+ cells (H69 cells) but not to CD56− cells (CHO cells).

FIG. 2 is a sensogram depicting the binding and dissociation of dAbs that bind CD38 (DOM11-3 and DOM11-30) as determined by surface plasmon resonance. The affinity (KD) of DOM11-3 was determined to be 250 nM and the affinity of DOM11-30 was determined to be 150 nM.

FIGS. 3A-3D are sensograms showing that dAbs that bind CD38 (DOM11-3, DOM11-30 and DOM11-23) bind to different epitopes on CD38. CD38 was immobilized on a surface plasmon resonance chip and a first anti-CD38 dAb was flowed over the surface (first arrow) then a second dAb was flowed over the surface (second arrow). The figures show that DOM11-30 bound to CD38 that had DOM11-3 already bound to it (FIG. 3A), DOM11-23 bound to CD38 that had DOM11-30 already bound to it (FIG. 3B), and, DOM11-3 bound to CD38 that had DOM11-23 already bound to it (FIG. 3C), demonstrating that these dAbs bind to different epitopes on the CD38 antigen. In contrast, flowing DOM11-30 over CD38 that had DOM11-30 already bound to it did not result in increased binding.

FIGS. 4A-4D are fluorescence dot plots showing that a ligand that bound CD38 and CD138 (DOM11-3/DOM12-45)(50 nM) selectively bound to double positive RPM182265 cells (CD38+/CD138+). DOM11-3/DOM12-45 did not substantially bind single positive Raji cells (CD38+/CD138−) or H647 cells (CD38−/Cd138+), or double negative cells (CCRF-CEM).

FIGS. 5A-5C are photomicrographs showing that the Raji (CD38+) cell line was labeled with a ligand that bound CD38 and CD138 (DOM11-3/DOM12-45) (500 nM). The ligand was visualized using secondary and tertiary reagents (FITC labeled) and a confocal microscope (Zeiss LSM510 META). Cells were maintained at 4° C. to inhibit internalization or at 37° C. to permit internalization. FIGS. 4A and 4B show that DOM11-3/DOM12-45 bound Raji cells but was not substantially internalized at 4° C. as shown by the lack of acid resistant fluorescence in FIG. 4B. In contrast, FIG. 4C shows acid resistant fluorescence at 37° C., demonstrating that DOM11-3/DOM12-45 was internalized.

FIGS. 6A-6B are fluorescent histograms showing that a ligand that bound CD38 and CD138 (DOM11-3/DOM12-45) bound the double positive myeloma cell line (OPM2, CD38+/CD138+). OPM2 cells were treated with DOM11-3/DOM12-45 at 4° C. or at 37° C. as described in FIGS. 5A-5C. Acid resistant fluorescence was detected at 37° C., demonstrating that the ligand was internalized. In contrast very little acid resistant fluorescence was detected at 4° C. or in cells treated with a dAb that does not bind CD38 or CD138 (Vk dummy), indicating that the ligand or dAb was not internalized.

FIG. 7 is a series of photomicrographs showing co-localization of a ligand that bound CD38 and CD138 (DOM11-3/DOM12-45) (green fluorescence) with the lysosomal marker, cathepsin B (red fluorescence), in Raji cells by confocal microscopy. Co-localized ligand and cathepsin B are shown in the overlay panels as yellow fluorescence.

FIGS. 8A-8E are fluorescence histograms showing that a ligand that bound CD38 and CD138 (DOM11-3/DOM12-45; da-dAb) that was pegylated with 5K (FIG. 8B), 20K (FIG. 5C), 30K (FIG. 8D) or 40K (FIG. 5E) linear PEG were internalized to about the same degree as unpegylated ligand (FIG. 8A) at 37° C. The figures show acid resistance fluorescence for each ligand at 37° C., demonstrating that the ligands were internalized.

FIGS. 9A-9D are fluorescence histograms showing that a ligand that bound CD38 and CD138 and contained a toxin (selenium) (DOM11-3/DOM12-45-Se) was internalized to the same degree as the corresponding ligand that did not contain a toxin (DOM11-3/DOM12-45) by OPM2 cells. The figures show acid resistance fluorescence for DOM11-3/DOM12-45-Se and for DOM11-3/DOM12-45 at 37° C., demonstrating that the ligands were internalized. In contrast ligands that did not bind CD38 or CD138 (Vk dummy/Vk dummy and Vk dummy/Vk dummy-Se) did not bind the cells or become internalized.

FIG. 10 is a histogram showing apoptosis of OPM2 mM cell line (CD38+/CD138+) and cells that did not express CD38 or CD138 (antigen-ve cell line) induced by camptothecin, a ligand that bound CD38 and CD138 and contained a toxin (selenium) (DOM11-3/DOM12-45-Se), a ligand that bound CD38 and CD138 (DOM11-3/DOM12-45), a ligand that did not bind CD38 and CD138 and contained a toxin (selenium) (Vkd Se), and a ligand that did not bind CD38 and CD138 (Vkd). The results show that DOM11-3/DOM12-45-Se selective induced apoptosis of double positive OPM2 mM cell line, whereas camptothecin induced apoptosis of both cell lines, and DOM11-3/DOM12-45, Vkd Se and Vkd did not induce apoptosis of either cell line.

FIG. 11 is a histogram showing that a ligand that bound CD38 and CD138 and contained a toxin (selenium) (DOM11-3/DOM12-45-Se; 38/138 Se) selectively induce cell death (reduced cell viability) of double positive OPM2 cells (CD38+/CD138+) but not single positive Raji cells (CD38+/Cd138−) or double negative CEM cells (CD38−/CD138−). The corresponding ligand that did not contain a toxin (DOM11-3/DOM12-45; 38/138−), a ligand that did not bind CD38 or CD138 (VKD/VKD−) and a ligand that did not bind CD38 or CD138 and contained a toxin (selenium) (VKD/VKD Se) did not reduce cell viability of any of the cell lines.

FIG. 12 is a fluorescence histogram showing that a ligand that bound CEA and CD56 (DOM14-23/DOM13-25) bound to double positive H69 cells (CEA+/CD56+), but that ligands that bound to CD56 but not CEA (DOM14-23/Vk dummy) and a ligand that bound CEA but not CD56 (Vk dummy/DOM13-25) did not bind H59 cells. Vk dummy is a dAb that does not bind CEA or CD56.

FIGS. 13A-13G illustrate the nucleotide sequences for several human anti-CD38 dAbs.

FIGS. 14A-14G illustrate the nucleotide sequences for several human anti-CD138 dAbs.

FIGS. 15A-150 illustrate the nucleotide sequences for several human anti-CEA dAbs.

FIGS. 16A-16K illustrate the nucleotide sequences for several human anti-CD56 dAbs.

FIGS. 17A-17F illustrate the amino acid sequences for several human anti-CD38 dAbs.

FIGS. 18A-18F illustrate the amino acid sequences for several human anti-CD138 dAbs.

FIGS. 19A-19G illustrate the amino acid sequences for several human anti-CEA dAbs.

FIGS. 20A-20E illustrate the amino acid sequences for several human anti-CD56 dAbs.

FIG. 21A is an alignment of the amino acid sequences of three Vκs that bind mouse serum albumin (MSA). The aligned amino acid sequences are from VκS designated MSA16, which is also referred to as DOM7m-16 (SEQ ID NO:541), MSA 12, which is also referred to as DOM7m-12 (SEQ ID NO:542), and MSA 26, which is also referred to as DOM7m-26 (SEQ ID NO:543).

FIG. 21B is an alignment of the amino acid sequences of six VκS that bind rat serum albumin (RSA). The aligned amino acid sequences are from Vκs designated DOM7r-1 (SEQ ID NO:544), DOM7r-3 (SEQ ID NO:545), DOM7r-4 (SEQ ID NO:546), DOM7r-5 (SEQ ID NO:547), DOM7r-7 (SEQ ID NO:548), and DOM7r-8 (SEQ ID NO:549).

FIG. 21C is an alignment of the amino acid sequences of six Vκs that bind human serum albumin (HSA). The aligned amino acid sequences are from VκS designated DOM7h-2 (SEQ ID NO:550), DOM7h-3 (SEQ ID NO:551), DOM7h-4 (SEQ ID NO:552), DOM7h-6 (SEQ ID NO:553), DOM7h-1 (SEQ ID NO:554), and DOM7h-7 (SEQ ID NO:555).

FIG. 21D is an alignment of the amino acid sequences of seven V_{H}s that bind human serum albumin and a consensus sequence (SEQ ID NO:556). The aligned sequences are from VHS designated DOM7h-22 (SEQ ID NO:557), DOM7h-23 (SEQ ID NO:558), DOM7h-24 (SEQ ID NO:559), DOM7h-25 (SEQ ID NO:560), DOM7h-26 (SEQ ID NO:561), DOM7h-21 (SEQ ID NO:562), and DOM7h-27 (SEQ ID NO:563).

FIG. 21E is an alignment of the amino acid sequences of three Vκs that bind human serum albumin and rat serum albumin. The aligned amino acid sequences are from VκS designated DOM7h-8 (SEQ ID NO:564), DOM7r-13 (SEQ ID NO:565), and DOM7r-14 (SEQ ID NO:566).

FIG. 22 is an illustration of the amino acid sequences of Vκs that bind rat serum albumin (RSA). The illustrated sequences are from Vκs designated DOM7r-15 (SEQ ID NO: 567), DOM7r-16 (SEQ ID NO: 568), DOM7r-17 (SEQ ID NO: 569), DOM7r-18 (SEQ ID NO: 570), DOM7r-19 (SEQ ID NO: 571).

FIGS. 23A-23B are an illustration of the amino acid sequences of the amino acid sequences of V_{H}s that bind rat serum albumin (RSA). The illustrated sequences are from V_{H}s designated DOM7r-20 (SEQ ID NO:572), DOM7r-21 (SEQ ID NO:573), DOM7r-22 (SEQ ID NO:574), DOM7r-23 (SEQ ID NO:575), DOM7r-24 (SEQ ID NO:576), DOM7r-25 (SEQ ID NO:577), DOM7r-26 (SEQ ID NO:578), DOM7r-27 (SEQ ID NO:579), DOM7r-28 (SEQ ID NO:580), DOM7r-29 (SEQ ID NO:581), DOM7r-30 (SEQ ID NO:582), DOM7r-31 (SEQ ID NO:583), DOM7r-32 (SEQ ID NO:584), and DOM7r-33 (SEQ ID NO:585).

FIG. 24 illustrates the amino acid sequences of several Camelid V_{HH}s that bind mouse serum albumin that are disclosed in WO 2004/041862. Sequence A (SEQ ID NO:586), Sequence B (SEQ ID NO:587), Sequence C (SEQ ID NO:588), Sequence D (SEQ ID NO:589), Sequence E (SEQ ID NO:590), Sequence F (SEQ ID NO:591), Sequence G (SEQ ID NO:592), Sequence H (SEQ ID NO:593), Sequence I (SEQ ID NO:594), Sequence J (SEQ ID NO:595), Sequence K (SEQ ID NO:596), Sequence L (SEQ ID NO:597), Sequence M (SEQ ID NO:598), Sequence N (SEQ ID NO:599), Sequence 0 (SEQ ID NO:600), Sequence P (SEQ ID NO:601), Sequence Q (SEQ ID NO:602).

FIG. 25 is a graph depicting the cell binding assay for dAb combinations on OMP2 multiple myeloma cells. The EC50 for DOM 11-3-1/DOM 12-45-2 was 13.81, 16.73 for DOM 11-3-15/DOM 12-45-2, 11.88 for DOM 11-3-20/DOM 12-45-2, 11.0 for DOM 11-3-23/DOM 12-45-2 and 44.35 for DOM 11-3/DOM 12-45.

FIGS. 26A-26D illustrate the nucleic acid sequence for several affinity matured human anti-CD38 dAbs.

FIGS. 27A-27C illustrate the nucleic acid sequence for several affinity matured human anti-CD38 dAbs.

FIGS. 28A-28G illustrate the nucleic acid sequence for several affinity matured human anti-CD138 dAbs.

FIG. 29 illustrate the anti-CD38/anti CD138 (DOM11-3/DOM 12-45) amino acid sequence (SEQ ID NO: 677), the anti-CD38/anti CD138 (DOM11-3/DOM 12-45) nucleic acid sequence (SEQ ID NO: 678), the Vk dummy animo acid sequence (SEQ ID NO: 679), and the Vk dummy nucleic acid sequence (SEQ ID NO: 680).

FIG. 30 illustrate nucleic acid sequences that encode several affinity matured human anti-CEA dAbs.

FIGS. 31A-31C illustrate the amino acid sequence and/or nucleic acid sequence of several human dAbs. The three alanine residues (AAA) at the C-terminus of the amino acid sequence of the DOM14-3A dAb, are not part of the amino acid sequence of the actual dAb but are encoded by the cloning site.

### DETAILED DESCRIPTION OF THE INVENTION

Within this specification embodiments have been described in a way which enables a clear and concise specification to be written, but it is intended and will be appreciated that embodiments may be variously combined or separated without parting from the invention.

As used herein, the term “ligand” refers to a polypeptide that comprises a first polypeptide domain which has a binding site that has binding specificity for a first cell surface target and a second polypeptide domain which has a binding site that has binding specificity for a second first cell surface target. The first cell surface target and the second cell surface target are not the same (i.e., are different targets (e.g., proteins)), but are both present (e.g., co-expressed) on a cell, such as a pathogenic cell as described herein. A ligand of the invention binds a cell that contains the first cell surface target and the second cell surface target more strongly (e.g., with greater avidity) than a cell that contains only one target. Accordingly, a ligand of the invention can selectively bind to a cell that contains the first cell surface target and the second cell surface target.

The ligands of the invention can bind to cell surface targets that are both present on normal cells, but that are present at higher levels on a pathogenic cell. In such circumstances, the ligand can be used to preferentially deliver a therapeutic agent (e.g., a toxin) to the pathogenic cell. For example, due to the higher level of cell surface targets on the pathogenic cell, more ligand will bind the pathogenic cell and be internalized than will bind and be internalized into the normal cell. Thus, an effective amount of toxin can be delivered preferentially to the pathogenic cell.

The ligands according to the invention preferably comprise immunoglobulin variable domains which have different binding specificities, and do not contain variable domain pairs which have the same specificity. Preferably each domain which has a binding site that has binding specificity for a cell surface target is an immunoglobulin single variable domain (e.g., immunoglobulin single heavy chain variable domain (e.g., V_{H}, V_{HH}) immunoglobulin single light chain variable domain (e.g., V_{L})) that has binding specificity for a desired cell surface target (e.g., a membrane protein, such as a receptor protein). Each polypeptide domain which has a binding site that has binding specificity for a cell surface target can also comprise one or more complementarity determining regions (CDRs) of an antibody or antibody fragment (e.g., an immunoglobulin single variable domain) that has binding specificity for a desired cell surface target in a suitable format, such that the binding domain has binding specificity for the cell surface target. For example, the CDRs can be grafted onto a suitable protein scaffold or skeleton, such as an affibody, an SpA scaffold, an LDL receptor class A domain, or an EGF domain. Further, the ligand can be bivalent (heterobivalent) or multivalent (heteromultivalent) as described herein. Thus, “ligands” include polypeptides that comprise two dAbs wherein each dAb binds to a different cell surface target. Ligands also include polypeptides that comprise at least two dAbs that bind different cell surface targets (or the CDRs of a dAbs) in a suitable format, such as an antibody format (e.g., IgG-like format, scFv, Fab, Fab′, F(ab′)₂) or a suitable protein scaffold or skeleton, such as an affibody, an SpA scaffold, an LDL receptor class A domain, an EGF domain, avimer and multispecific ligands as described herein. The polypeptide domain which has a binding site that has binding specificity for a cell surface target (i.e., first or second cell surface target) can also be a protein domain comprising a binding site for a desired target, e.g., a protein domain selected from an affibody, an SpA domain, an LDL receptor class A domain, an avimer (see, e.g., U.S. Patent Application Publication Nos. 2005/0053973, 2005/0089932, 2005/0164301).

As used herein, the phrase “target” refers to a biological molecule (e.g., peptide, polypeptide, protein, lipid, carbohydrate) to which a polypeptide domain which has a binding site can bind. The target can be, for example, an intracellular target (e.g., an intracellular protein target) or a cell surface target (e.g., a membrane protein, a receptor protein). Preferably, a target is a cell surface target, such as a cell surface protein. Preferably, the first cell surface target and second cell surface target are both present on a pathogenic cell (e.g., a cancer cell, a tumor cell). For example, the first cell surface target and the second cell surface target can be co-expressed on a cell (e.g., pathogenic cell). The first cell surface target and the second cell surface target can be individually present on certain normal cells, and can both be present on pathogenic cells (e.g., co-expressed on cancer cells, co-expressed on tumor cells).

Certain suitable targets (e.g., certain first cell surface targets and certain second cell surface targets) might both be present on normal cells. In such situations, the targets are expressed at low levels on normal cells but expressed at higher levels on, for example, pathogenic cells. For example, a first cell surface target and a second cell surface target can be present on a pathogenic cell at levels that are at least about 2, about 3, about 4, about 5, about 6, about 7, about 8, about 9, or at least about 10 times higher than the levels on normal cells. The level of a target on a cell (e.g., amount of target on the surface of a cell) can be determined using a variety of suitable methods, such as antibody binding and flow cytometry.

As used herein, the term “pathogenic cell” refers to a cell with altered cellular physiology that can produce or contribute to the production of a pathogenic condition (e.g., cancer). A pathogenic cell can be, for example, a cell that harbors one or more mutations that dysregulate the normal cellular processes of cellular division, proliferation, differentiation, senescence and/or death. Particular pathogenic cells include cancer cells, such as carcinoma cells, lymphoma cells, myeloma cells, sarcoma cells and the like.

The phrase “immunoglobulin single variable domain” refers to an antibody variable region (V_{H}, V_{HH}, V_{L}) that specifically binds a target, antigen or epitope independently of other V domains; however, as the term is used herein, an immunoglobulin single variable domain can be present in a format (e.g., hetero-multimer) with other variable regions or variable domains where the other regions or domains are not required for antigen binding by the single immunoglobulin variable domain (i.e., where the immunoglobulin single variable domain binds antigen independently of the additional variable domains). Each “Immunoglobulin single variable domain” encompasses not only an isolated antibody single variable domain polypeptide, but also larger polypeptides that comprise one or more monomers of an antibody single variable domain polypeptide sequence. A “domain antibody” or “dAb” is the same as an “immunoglobulin single variable domain” polypeptide as the term is used herein. An immunoglobulin single variable domain polypeptide, as used herein refers to a mammalian immunoglobulin single variable domain polypeptide, preferably human, but also includes rodent (for example, as disclosed in WO 00/29004, the contents of which are incorporated herein by reference in their entirety) or camelid V_{HH} dAbs. As used herein, camelid dAbs are immunoglobulin single variable domain polypeptides which are derived from species including camel, llama, alpaca, dromedary, and guanaco, and comprise heavy chain antibodies naturally devoid of light chain (V_{HH}). Similar dAbs, can be obtained for single chain antibodies from other species, such as nurse shark. Preferred ligands comprise at least two different immunoglobulin single variable domain polypeptides or at least two different dAbs.

As used herein, “selectively binds” refers to the ability of the ligand of the invention to preferentially bind double positive cells over single positive cells. For example, the ligand of the invention can bind to double positive cells but not substantially bind to single positive cells. A ligand “does not substantially bind” to single positive cells when the amount of binding to single positive cells is no more than about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2% or about 1%, of the binding to double positive cells under the same binding conditions. Selective binding can be influenced by, for example, the affinity and avidity of the ligand and the concentration of ligand. The person of ordinary skill in the art can determine appropriate conditions under which the ligands of the invention selectively bind double positive cells using any suitable methods, such as titration of ligand in a suitable cell binding assay.

As used herein, the term “double positive” refers to a cell that contains two different cell surface targets (different target species) that are bound by a ligand of the invention. Ligands of the invention bind double positive cells with high avidity. As used herein, the term “single positive” refers to a cell that contains only one cell surface target that is bound by a ligand of the invention.

As used herein, the terms “internalize,” “internalized,” and “internalization,” and related variant terms, refer to the cellular processes by which ligands are brought into the cell (e.g., endocytosis) upon binding to the first cell surface target and the second cell surface target. Internalization can be mediated by clathrin-coated pit endocytosis following ligand induced clustering of cell surface targets. Once endocytosed, the ligands may be delivered to the lysosomal compartment of the cell, wherein cellular enzymes such as cathepsin B can cleave portions of the ligand (e.g., cleave a linker to release a toxin from the ligand).

“Affinity” and “avidity” are terms of art that describe the strength of a binding interaction. With respect to the ligands of the invention, avidity refers to the overall strength of binding between the targets (e.g., first cell surface target and second cell surface target) on the cell and the ligand. Avidity is more than the sum of the individual affinities for the individual targets.

As used herein, “toxin moiety” refers to a moiety that comprises a toxin. A toxin is an agent that has deleterious effects on or alters cellular physiology (e.g., causes cellular necrosis, apoptosis or inhibits cellular division).

As used herein, the term “dose” refers to the quantity of ligand administered to a subject all at one time (unit dose), or in two or more administrations over a defined time interval. For example, dose can refer to the quantity of ligand (e.g., ligand comprising an immunoglobulin single variable domain that binds CEA and an immunoglobulin single variable domain that binds CD56) administered to a subject over the course of one day (24 hours) (daily dose), two days, one week, two weeks, three weeks or one or more months (e.g., by a single administration, or by two or more administrations). The interval between doses can be any desired amount of time.

As used herein “Complementary” refers to when two immunoglobulin domains belong to families of structures which form cognate pairs or groups or are derived from such families and retain this feature. For example, a V_{H} domain and a V_{L} domain of an antibody are complementary; two V_{H} domains are not complementary, and two V_{L} domains are not complementary. Complementary domains may be found in other members of the immunoglobulin superfamily, such as the V_{α} and V_{β} (or γ and δ) domains of the T-cell receptor. Domains which are artificial, such as domains based on protein scaffolds which do not bind epitopes unless engineered to do so, are non-complementary. Likewise, two domains based on (for example) an immunoglobulin domain and a fibronectin domain are not complementary.

As used herein, “Immunoglobulin” refers to a family of polypeptides which retain the immunoglobulin fold characteristic of antibody molecules, which contains two β sheets and, usually, a conserved disulphide bond. Members of the immunoglobulin superfamily are involved in many aspects of cellular and non-cellular interactions in vivo, including widespread roles in the immune system (for example, antibodies, T-cell receptor molecules and the like), involvement in cell adhesion (for example the ICAM molecules) and intracellular signaling (for example, receptor molecules, such as the PDGF receptor). The present invention is applicable to all immunoglobulin superfamily molecules which possess binding domains. Preferably, the present invention relates to antibodies.

As used herein “domain” refers to a folded protein structure which retains its tertiary structure independently of the rest of the protein. Generally, domains are responsible for discrete functional properties of proteins, and in many cases may be added, removed or transferred to other proteins without loss of function of the remainder of the protein and/or of the domain. By single antibody variable domain is meant a folded polypeptide domain comprising sequences characteristic of antibody variable domains. It therefore includes complete antibody variable domains and modified variable domains, for example in which one or more loops have been replaced by sequences which are not characteristic of antibody variable domains, or antibody variable domains which have been truncated or comprise N- or C-terminal extensions, as well as folded fragments of variable domains which retain at least in part the binding activity and specificity of the full-length domain. Thus, each ligand comprises at least two different domains.

“Repertoire” A collection of diverse variants, for example polypeptide variants which differ in their primary sequence. A library used in the present invention will encompass a repertoire of polypeptides comprising at least 1000 members.

“Library” The term library refers to a mixture of heterogeneous polypeptides or nucleic acids. The library is composed of members, each of which have a single polypeptide or nucleic acid sequence. To this extent, library is synonymous with repertoire. Sequence differences between library members are responsible for the diversity present in the library. The library may take the form of a simple mixture of polypeptides or nucleic acids, or may be in the form of organisms or cells, for example bacteria, viruses, animal or plant cells and the like, transformed with a library of nucleic acids. Preferably, each individual organism or cell contains only one or a limited number of library members. Advantageously, the nucleic acids are incorporated into expression vectors, in order to allow expression of the polypeptides encoded by the nucleic acids. In a preferred aspect, therefore, a library may take the form of a population of host organisms, each organism containing one or more copies of an expression vector containing a single member of the library in nucleic acid form which can be expressed to produce its corresponding polypeptide member. Thus, the population of host organisms has the potential to encode a large repertoire of genetically diverse polypeptide variants.

As used herein an antibody refers to IgG, IgM, IgA, IgD or IgE or a fragment (such as a Fab, F(ab′)₂, Fv, disulphide linked Fv, scFv, closed conformation multispecific antibody, disulphide-linked scfv, diabody) whether derived from any species naturally producing an antibody, or created by recombinant DNA technology; whether isolated from serum, B-cells, hybridomas, transfectomas, yeast or bacteria.

As described herein an “antigen” is a molecule that is bound by a binding domain according to the present invention. Typically, antigens are bound by antibody ligands and are capable of raising an antibody response in vivo. It may be a polypeptide, protein, nucleic acid or other molecule. Generally, the dual-specific ligands according to the invention are selected for target specificity against two particular targets (e.g., antigens). In the case of conventional antibodies and fragments thereof, the antibody binding site defined by the variable loops (L1, L2, L3 and H1, H2, H3) is capable of binding to the antigen.

An “epitope” is a unit of structure conventionally bound by an immunoglobulin V_{H}/V_{L} pair. Epitopes define the minimum binding site for an antibody, and thus represent the target of specificity of an antibody. In the case of a single domain antibody, an epitope represents the unit of structure bound by a variable domain in isolation.

“Universal framework” refers to a single antibody framework sequence corresponding to the regions of an antibody conserved in sequence as defined by Kabat (“Sequences of Proteins of Immunological Interest”, US Department of Health and Human Services) or corresponding to the human germline immunoglobulin repertoire or structure as defined by Chothia and Lesk, J. Mol. Biol. 196:910-917 (1987). The invention provides for the use of a single framework, or a set of such frameworks, which has been found to permit the derivation of virtually any binding specificity though variation in the hypervariable regions alone.

The phrase, “half-life,” refers to the time taken for the serum concentration of the ligand to reduce by 50%, in vivo, for example due to degradation of the ligand and/or clearance or sequestration of the dual-specific ligand by natural mechanisms. The ligands of the invention are stabilized in vivo and their half-life increased by binding to molecules which resist degradation and/or clearance or sequestration. Typically, such molecules are naturally occurring proteins which themselves have a long half-life in vivo. The half-life of a ligand is increased if its functional activity persists, in vivo, for a longer period than a similar ligand which is not specific for the half-life increasing molecule. Thus a ligand specific for HSA and two target molecules is compared with the same ligand wherein the specificity to HAS is not present, that is does not bind HAS but binds another molecule. For example, it may bind a third target on the cell. Typically, the half-life is increased by 10%, 20%, 30%, 40%, 50% or more. Increases in the range of 2×, 3×, 4×, 5×, 10×, 20×, 30×, 40×, 50× or more of the half-life are possible. Alternatively, or in addition, increases in the range of up to 30×, 40×, 50×, 60×, 70×, 80×, 90×, 100×, 150× of the half-life are possible.

As referred to herein, the term “competes” means that the binding of a first target to its cognate target binding domain is inhibited when a second target is bound to its cognate target binding domain. For example, binding may be inhibited sterically, for example by physical blocking of a binding domain or by alteration of the structure or environment of a binding domain such that its affinity or avidity for a target is reduced.

As used herein, the terms “low stringency,” “medium stringency,” “high stringency,” or “very high stringency conditions” describe conditions for nucleic acid hybridization and washing. Guidance for performing hybridization reactions can be found in Current Protocols in Molecular Biology, John Wiley & Sons, N.Y. (1989), 6.3.1-6.3.6, which is incorporated herein by reference in its entirety. Aqueous and nonaqueous methods are described in that reference and either can be used. Specific hybridization conditions referred to herein are as follows: (1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); (2) medium stringency hybridization conditions in 6×SSC at about 45° C., followed by one or more washes in 0.2×SSC, 0.1% SDS at 60° C.; (3) high stringency hybridization conditions in 6×SSC at about 45° C., followed by one or more washes in 0.2×SSC, 0.1% SDS at 65° C.; and preferably (4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

Sequences similar or homologous (e.g., at least about 70% sequence identity) to the sequences disclosed herein are also part of the invention. In some embodiments, the sequence identity at the amino acid level can be about 80%, 85%, 90%, 91%, 92%, 93%, 94%, 95%, 96%, 97%, 98%, 99% or higher. At the nucleic acid level, the sequence identity can be about 70%, 75%, 80%, 85%, 90%, 91%, 92%, 93%, 94%, 95%, 96%, 97%, 98%, 99% or higher. Alternatively, substantial identity exists when the nucleic acid segments will hybridize under selective hybridization conditions (e.g., very high stringency hybridization conditions), to the complement of the strand. The nucleic acids may be present in whole cells, in a cell lysate, or in a partially purified or substantially pure form.

Calculations of “homology” or “sequence identity” or “similarity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “homology” is equivalent to amino acid or nucleic acid “identity”). The percent identity between the two sequences is a function of the number of identical positions shared by the sequences, taking into account the number of gaps, and the length of each gap, which need to be introduced for optimal alignment of the two sequences.

Amino acid and nucleotide sequence alignments and homology, similarity or identity, as defined herein are preferably prepared and determined using the algorithm BLAST 2 Sequences, using default parameters (Tatusova, T. A. et al., FEMS Microbiol Lett, 174:187-188 (1999)). Alternatively, the BLAST algorithm (version 2.0) is employed for sequence alignment, with parameters set to default values. BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul, 1990, Proc. Natl. Acad. Sci. USA 87(6):2264-8.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4ₜₕ Ed, John Wiley & Sons, Inc. which are incorporated herein by reference) and chemical methods.

The invention relates to ligands that bind two cell surface targets that are present on a cell. For example, the ligand can comprise a first polypeptide domain having a binding site with binding specificity for a first cell surface target and a second polypeptide domain having a binding site with binding specificity for a second cell surface target. Preferably, the first polypeptide domain (e.g., immunoglobulin single variable domain) binds said first cell surface target with low affinity and said second polypeptide domain (immunoglobulin single variable domain) binds said second cell surface target with low affinity.

As described and exemplified herein, such ligands can selectively bind to double positive cells that contain both the first cell surface target and the second cell surface target. Accordingly, polypeptides that bind a desired cell surface antigen with low affinity, such as antibodies and antigen-binding fragments of antigens, can be formatted into ligands as described herein to provide agents that can selectively bind to double positive cells.

The ligands of the invention provide several advantages. For example, as described herein, the ligands that bind two different cell surface targets can be internalized into cells upon binding the two targets on the surface of a cell. Accordingly, the ligands can be used to deliver a therapeutic agent, such as a toxin, to a double positive cell that expresses a first cell surface target and a second cell surface target, such as a cancer cell. Because the ligand can selectively bind double positive cells, possible undesirable effects that might result from delivering a therapeutic agent to a single positive cell (e.g., side effects such as immunosuppression) can be avoided using the ligands of the invention.

The ligands of the invention can bind to cell surface targets that are both present on normal cells, but that are present at higher levels on a pathogenic cell. In such circumstances, the ligand can be used to preferentially deliver a therapeutic agent (e.g., a toxin) to the pathogenic cell. For example, due to the higher level of cell surface targets on the pathogenic cell, more ligand will bind the pathogenic cell and be internalized than will bind and be internalized into the normal cell. Thus, an effective amount of toxin can be delivered preferentially to the pathogenic cell.

Further, as described herein, the ligand can be tailored to have a desired in vivo serum half-life. Thus, the ligands can be used to control, reduce, or eliminate general toxicity of therapeutic agents, such as cytotoxin used to treat cancer.

Generally both of the cell surface targets that the ligand binds are present on a pathogenic cell, but are not both present on normal cells. As shown herein, in such situations, the ligand can be used at a concentration that results in selective binding to pathogenic cells that contain both cell surface targets (at a concentration wherein the ligand does not substantially bind single positive normal cells).

Certain normal cells may have both cell surface targets that are bound by a ligand of the invention present on their cell surfaces, but the targets are present at higher levels on the surface of a pathogenic cell (e.g., a cancer cell). Preferably, both cell surface targets are not substantially present on the surface of normal cells. In these circumstances, the ligand can be used at a concentration that results in selective binding to pathogenic cells that contain both cell surface targets (at a concentration wherein the ligand does not substantially bind the normal cell that contains low levels of the cell surface targets).

Preferred ligands comprise a first immunoglobulin single variable domain with binding specificity for a first cell surface target and a second immunoglobulin single domain with binding specificity for a second cell surface target. In preferred embodiments, the first immunoglobulin single variable domain has a binding site with binding specificity for a cell surface target selected from the group consisting of CD38, CD138, carcinoembrionic antigen (CEA), CD56, vascular endothelial growth factor (VEGF), epidermal growth factor receptor (EGFR), and HER2. In particularly preferred embodiments, the second immunoglobulin single variable domain has a binding site with binding specificity for a cell surface target selected from the group consisting of CD38, CD138, CEA, CD56, VEGF, EGFR, and HER2, with the proviso that said first immunoglobulin single variable domain and said second immunoglobulin single variable domain do not bind the same cell surface target.

The ligand of the invention can be formatted as described herein. For example, the ligand of the invention can be formatted to tailor in vivo serum half-life. If desired, the ligand can further comprise a toxin or a toxin moiety as described herein. In some embodiments, the ligand comprises a surface active toxin, such as a free radical generator (e.g., selenium containing toxin) or a radionuclide. In other embodiments, the toxin or toxin moiety is a polypeptide domain (e.g., a dAb) having a binding site with binding specificity for an intracellular target.

TABLE 1
Target specificities for ligands

| | | SECOND CELL |
|---|---|---|
| FIRST CELL | | SURFACE TARGET |
| SURFACE TARGET | DISEASE | VARIATIONS |
| CD38 | Cancer | CD138 |
| | (e.g., multiple myeloma) | CD56 |
| CD138 | Cancer | CD38 |
| | (e.g., multiple myeloma) | CD56 |
| CD138 | Cancer | CD56 |
| | (e.g., lung cancer, small | CEA |
| | cell lung carcinoma) | |
| CD56 | Cancer | CD138 |
| | (e.g., lung cancer, small | CEA |
| | cell lung carcinoma) | |
| EGFR | Cancer | HER2/neu |
| | (e.g., lung cancer, small | VEGF |
| | cell lung carcinoma, | |
| | brest cancer, colorectal | |
| | cancer) | |
| VEGF | Cancer | EGFR |
| | (e.g., metastatic cancer, | HER2 |
| | tumor angiogenesis) | |

Those skilled in the art will appreciate that the target combinations provided in Table 1 and those provided in the EXAMPLES represent a mere sample of suitable combinations for use according to the invention.

TABLE 2

| Target | Other names | function | Ref./Assession No. |
|---|---|---|---|
| CD38 | T10 ADP-ribosyl | CD38 is a novel multifunctional | Ferrero E J. Leukoc. Biol. |
| | cyclase/cyclic ADP- | ectoenzyme widely expressed in cells | 1999 65: 151 |
| | ribose hydrolase | and tissues especially in leukocytes. | Genebank Assession No.: |
| | | CD38 also functions in cell adhesion, | P28907 |
| | | signal transduction and calcium | |
| | | signaling | |
| CD56 | Leu-19, NKH1, | mediates homophilic adhesion in | Thiery JP et al. Proc Natl |
| | neural cell adhesion | certain cell types | Acad Sci USA 1982 |
| | molecule, NCAM | | 79: 6737 |
| | | | Genebank Assession No.: |
| | | | P13592 |
| CD138 | heparan sulfate | The syndecans mediate cell binding, | J Biol Regul Homeost |
| | proteoglycan; | cell signaling, and cytoskeletal | Agents, 2002 Apr- |
| | syndecan-1 | organization and syndecan receptors | Jun; 16(2): 152-5 |
| | | are required for internalization of the | Genebank Assession No.: |
| | | HIV-1 tat protein | P18827 |
| CEA | Carcinoembryonic | complex immunoreactive glycoprotein | Duffy, M. J., Clin Chem. |
| | antigen | | 2001 Apr; 47(4): 624-30 |
| | | | Genebank Assession No.: |
| | | | P06731 |
| EGFR | ErbB family of | receptor tyrosine kinases are important | Baselga and Mendelsohn |
| | receptor tyrosine | mediators of cell growth, | Pharmac. Ther. 64: 127-154 |
| | kinase | differentiation and survival | (1994). |
| | | | Genebank Assession No.: |
| | | | AAB19486 |
| HER2 | heregulin 2 EC | Essential component of a neuregulin- | Science 230 (4730), 1132- |
| | 2.7.1.112 | receptor complex, althought | 1139 (1985) Genebank |
| | p185erbB2 | neuregulins do not interact with it | Assession No.: NP_004439 |
| | C-erbB-2 | alone. GP30 is a potential ligand for | |
| | NEU proto- | this receptor. Not activated by EGF, | |
| | oncogene | TGF-alpha and amphiregulin | |
| | Tyrosine kinase- | | |
| | type cell surface | | |
| | receptor HER2 | | |
| | MLN 19 | | |
| VEGF | Vascular | inducer of angiogenesis | Genebank Assession No.: |
| | permeability factor | | NP_001020537 |

### Ligand Formats

The ligand of the invention can be formatted as a dual specific ligand as described herein. The ligand can also be formatted as a multispecific ligand, for example as described in WO 03/002609, the entire teachings of which are incorporated herein by reference. Such dual specific ligands comprise immunoglobulin single variable domains that have different binding specificities. Such dual specific ligands can comprise combinations of heavy and light chain domains. For example, the dual specific ligand may comprise a V_{H} domain and a V_{L} domain, which may be linked together in the form of an scFv (e.g., using a suitable linker such as Gly₄Ser), or formatted into a bispecific antibody or antigen-binding fragment thereof (e.g. F(ab′)₂ fragment). The dual specific ligands do not comprise complementary V_{H}/V_{L} pairs which form a conventional two chain antibody antigen-binding site that binds antigen or epitope co-operatively. Instead, the dual format ligands comprise a V_{H}/V_{L} complementary pair, wherein the V domains have different binding specificities.

In addition, the dual specific ligands may comprise one or more C_{H} or C_{L} domains if desired. A hinge region domain may also be included if desired. Such combinations of domains may, for example, mimic natural antibodies, such as IgG or IgM, or fragments thereof, such as Fv, scFv, Fab or F(ab′)₂ molecules. Other structures, such as a single aim of an IgG molecule comprising V_{H}, V_{L}, C_{H}1 and C_{L} domains, are envisaged. Preferably, the dual specific ligand of the invention comprises only two variable domains although several such ligands may be incorporated together into the same protein, for example two such ligands can be incorporated into an IgG or a multimeric immunoglobulin, such as IgM. Alternatively, in another embodiment a plurality of dual specific ligands are combined to form a multimer. For example, two different dual specific ligands are combined to create a tetra-specific molecule. It will be appreciated by one skilled in the art that the light and heavy variable regions of a dual-specific ligand produced according to the method of the present invention may be on the same polypeptide chain, or alternatively, on different polypeptide chains. In the case that the variable regions are on different polypeptide chains, then they may be linked via a linker, generally a flexible linker (such as a polypeptide chain), a chemical linking group, or any other method known in the art.

Ligands can be formatted as bi- or multispecific antibodies or antibody fragments or into bi- or multispecific non-antibody structures. Suitable formats include, any suitable polypeptide structure in which an antibody variable domain or one or more of the CDRs thereof can be incorporated so as to confer binding specificity for antigen on the structure. A variety of suitable antibody formats are known in the art, such as, bispecific IgG-like formats (e.g., chimeric antibodies, humanized antibodies, human antibodies, single chain antibodies, heterodimers of antibody heavy chains and/or light chains, antigen-binding fragments of any of the foregoing (e.g., a Fv fragment (e.g., single chain Fv (scFv), a disulfide bonded Fv), a Fab fragment, a Fab′ fragment, a F(ab′)₂ fragment), a single variable domain (e.g., V_{H}, V_{L}, V_{HH}), a dAb, and modified versions of any of the foregoing (e.g., modified by the covalent attachment of polyalkylene glycol (e.g., polyethylene glycol, polypropylene glycol, polybutylene glycol) or other suitable polymer). See, PCT/GB03/002804, filed Jun. 30, 2003, which designated the United States, (WO 2004/081026) regarding PEGylated single variable domains and dAbs, suitable methods for preparing same, increased in vivo half-life of the PEGylated single variable domains and dAb monomers and multimers, suitable PEGs, preferred hydrodynamic sizes of PEGs, and preferred hydrodynamic sizes of PEGylated single variable domains and dAb monomers and multimers. The entire teaching of PCT/GB03/002804 (WO 2004/081026), including the portions referred to above, are incorporated herein by reference.

The ligand can be formatted using a suitable linker such as (Gly₄Ser)ₙ, where n=from 1 to 8, e.g., 2, 3, 4, 5, 6 or 7. If desired, ligands, including dAb monomers, dimers and trimers, can be linked to an antibody Fc region, comprising one or both Of C_{H}2 and C_{H}3 domains, and optionally a hinge region. For example, vectors encoding ligands linked as a single nucleotide sequence to an Fc region may be used to prepare such polypeptides.

Ligands and dAb monomers can also be combined and/or formatted into non-antibody multi-ligand structures to form multivalent complexes, which bind target molecules with the same epitope, thereby providing superior avidity. For example natural bacterial receptors such as SpA can been used as scaffolds for the grafting of CDRs to generate ligands which bind specifically to one or more epitopes. Details of this procedure are described in U.S. Pat. No. 5,831,012. Other suitable scaffolds include those based on fibronectin and affibodies. Details of suitable procedures are described in WO 98/58965. Other suitable scaffolds include lipocallin and CTLA4, as described in van den Beuken et al., J. Mol. Biol. 310:591-601 (2001), and scaffolds such as those described in WO 00/69907 (Medical Research Council), which are based for example on the ring structure of bacterial GroEL or other chaperone polypeptides. Protein scaffolds may be combined, for example, CDRs may be grafted on to a CTLA4 scaffold and used together with immunoglobulin V_{H} or V_{L} domains to form a ligand. Likewise, fibronectin, lipocallin and other scaffolds may be combined

A variety of suitable methods for preparing any desired format are known in the art. For example, antibody chains and formats (e.g., bispecific IgG-like formats, chimeric antibodies, humanized antibodies, human antibodies, single chain antibodies, homodimers and heterodimers of antibody heavy chains and/or light chains) can be prepared by expression of suitable expression constructs and/or culture of suitable cells (e.g., hybridomas, heterohybridomas, recombinant host cells containing recombinant constructs encoding the format). Further, formats such as antigen-binding fragments of antibodies or antibody chains (e.g., bispecific binding fragments, such as a Fv fragment (e.g., single chain Fv (scFv), a disulfide bonded Fv), a Fab fragment, a Fab′ fragment, a F(ab′)₂ fragment), can be prepared by expression of suitable expression constructs or by enzymatic digestion of antibodies, for example using papain or pepsin.

The ligand can be formatted as a multispecific ligand, for example as described in WO 03/002609, the entire teachings of which are incorporated herein by reference. Such a multispecific ligand possesses more than one epitope binding specificity. Generally, the multi-specific ligand comprises two or more epitope binding domains, such as dAbs or non-antibody protein domain comprising a binding site for an epitope, e.g., an affibody, an SpA domain, an LDL receptor class A domain, an EGF domain, an avimer. Multispecific ligands can be formatted further as described herein.

In some embodiments, the ligand is an IgG-like format. Such formats have the conventional four chain structure of an IgG molecule (2 heavy chains and two light chains), in which one or more of the variable regions (V_{H} and or V_{L}) have been replaced with a dAb or single variable domain of a desired specificity. Preferably, each of the variable regions (2 V_{H} regions and 2 V_{L} regions) is replaced with a dAb or single variable domain. The dAb(s) or single variable domain(s) that are included in an IgG-like format can have the same specificity or different specificities. In some embodiments, the IgG-like format is tetravalent and can have one, two, three or four specificities. For example, the IgG-like format can be monospecific and comprises 4 dAbs that have the same specificity; bispecific and comprises 3 dAbs that have the same specificity and another dAb that has a different specificity; bispecific and comprise two dAbs that have the same specificity and two dAbs that have a common but different specificity; trispecific and comprises first and second dAbs that have the same specificity, a third dAbs with a different specificity and a fourth dAb with a different specificity from the first, second and third dAbs; or tetraspecific and comprise four dAbs that each have a different specificity. Antigen-binding fragments of IgG-like formats (e.g., Fab, F(ab′)₂, Fab′, Fv, scFv) can be prepared.

The ligands of the invention can be formatted as a fusion protein that contains a first immunoglobulin single variable domain that is fused directly to a second immunoglobulin single variable domain. If desired such a format can further comprise a half-life extending moiety. For example, the ligand can comprise a first immunoglobulin single variable domain, that is fused directly to a second immunoglobulin single variable domain, that is fused directly to an immunoglobulin single variable domain that binds serum albumin.

Generally the orientation of the polypeptide domains that have a binding site with binding specificity for a cell surface target and whether the ligand comprises a linker is a matter of design choice. However, some orientations, with or without linkers, may provide better binding characteristics than other orientations. All orientations (e.g., dAb1-linker-dAb2; dAb2-linker-dAb1) are encompassed by the invention, and ligands that contain an orientation that provides desired binding characteristics can be easily identified by screening.

### Half-Life Extended Formats

The ligand, and dAb monomers disclosed herein, can be formatted to extend its in vivo serum half-life. Increased in vivo half-life is useful in in vivo applications of immunoglobulins, especially antibodies and most especially antibody fragments of small size such as dAbs. Such fragments (Fvs, disulphide bonded Fvs, Fabs, scFvs, dAbs) are rapidly cleared from the body, which can limit clinical applications.

A ligand can be formatted as a larger antigen-binding fragment of an antibody or as an antibody (e.g., formatted as a Fab, Fab′, F(ab)₂, F(ab′)₂, IgG, scFv) that has larger hydrodynamic size. Ligands can also be formatted to have a larger hydrodynamic size, for example, by attachment of a polyalkyleneglycol group (e.g. polyethyleneglycol (PEG) group, polypropylene glycol, polybutylene glycol), serum albumin, transferrin, transferrin receptor or at least the transferrin-binding portion thereof, an antibody Fc region, or by conjugation to an antibody domain. In some embodiments, the ligand is PEGylated. Preferably the PEGylated ligand binds a double positive cell with substantially the same avidity as the same ligand that is not PEGylated. For example, the ligand can be a PEGylated ligand comprising a dAb that binds CD38 and a second dAb that binds CD138, wherein the PEGylated ligand binds a CD38⁺ CD138⁺ cell with an avidity that differs from the avidity of ligand in unPEGylated form by no more than a factor of about 1000, preferably no more than a factor of about 100, more preferably no more than a factor of about 10, or with avidity substantially unchanged relative to the unPEGylated form. See, PCT/GB03/002804, filed Jun. 30, 2003, which designated the United States, (WO 2004/081026) regarding PEGylated single variable domains and dAbs, suitable methods for preparing same, increased in vivo half-life of the PEGylated single variable domains and dAb monomers and multimers, suitable PEGs, preferred hydrodynamic sizes of PEGs, and preferred hydrodynamic sizes of PEGylated single variable domains and dAb monomers and multimers. The entire teaching of PCT/GB03/002804 (WO 2004/081026), including the portions referred to above, are incorporated herein by reference.

Hydrodynamic size of the ligands (e.g., dAb monomers and multimers) of the invention may be determined using methods which are well known in the art. For example, gel filtration chromatography may be used to determine the hydrodynamic size of a ligand. Suitable gel filtration matrices for determining the hydrodynamic sizes of ligands, such as cross-linked agarose matrices, are well known and readily available.

The size of a ligand format (e.g., the size of a PEG moiety attached to a dAb monomer), can be varied depending on the desired application. For example, where the ligand is intended to leave the circulation and enter into peripheral tissues, it is desirable to keep the hydrodynamic size of the ligand low to facilitate extravazation from the blood stream. Alternatively, where it is desired to have the ligand remain in the systemic circulation for a longer period of time the size of the ligand can be increased, for example by formatting as an Ig-like protein or by addition of a 30 to 60 kDa PEG moiety (e.g., linear or branched 30 to 40 kDa PEG, such as addition of two 20 kDa PEG moieties.) The size of the ligand format can be tailored to achieve a desired in vivo serum half-life, for example to control exposure to a toxin and/or to reduce side effects of toxic agents.

The hydrodynaminc size of ligand and its serum half-life can also be increased by conjugating or linking the ligand to a binding domain that binds an antigen or epitope that increases half-life in vivo, as described herein. For example, the ligand (e.g., dAb monomer) can be conjugated or linked to an anti-serum albumin or anti-neonatal Fc receptor antibody or antibody fragment, (e.g., an anti-SA or anti-neonatal Fc receptor dAb, Fab, Fab′ or scFv), or to an anti-SA affibody or anti-neonatal Fc receptor affibody.

Examples of suitable albumin, albumin fragments or albumin variants for use in a ligand according to the invention are described in WO 2005/077042A2, which is incorporated herein by reference in its entirety. In particular, the following albumin, albumin fragments or albumin variants can be used in the present invention:
SEQ ID NO: 1 as disclosed in WO 2005/077042A2, this sequence being explicitly incorporated into the present disclosure by reference;
Albumin fragment or variant comprising or consisting of amino acids 1-387 of SEQ ID NO:1 in WO 2005/077042A2;
Albumin, or fragment or variant thereof, comprising an amino acid sequence selected from the group consisting of: (a) amino acids 54 to 61 of SEQ ID NO:1 in WO 2005/077042A2; (b) amino acids 76 to 89 of SEQ ID NO:1 in WO 2005/077042A2; (c) amino acids 92 to 100 of SEQ ID NO:1 in WO 2005/077042A2; (d) amino acids 170 to 176 of SEQ ID NO:1 in WO 2005/077042A2; (e) amino acids 247 to 252 of SEQ ID NO:1 in WO 2005/077042A2; (f) amino acids 266 to 277 of SEQ ID NO:1 in WO 2005/077042A2; (g) amino acids 280 to 288 of SEQ ID NO:1 in WO 2005/077042A2; (h) amino acids 362 to 368 of SEQ ID NO:1 in WO 2005/077042A2; (i) amino acids 439 to 447 of SEQ ID NO:1 in WO 2005/077042A2 (j) amino acids 462 to 475 of SEQ ID NO:1 in WO 2005/077042A2; (k) amino acids 478 to 486 of SEQ ID NO:1 in WO 2005/077042A2; and (1) amino acids 560 to 566 of SEQ ID NO:1 in WO 2005/077042A2.

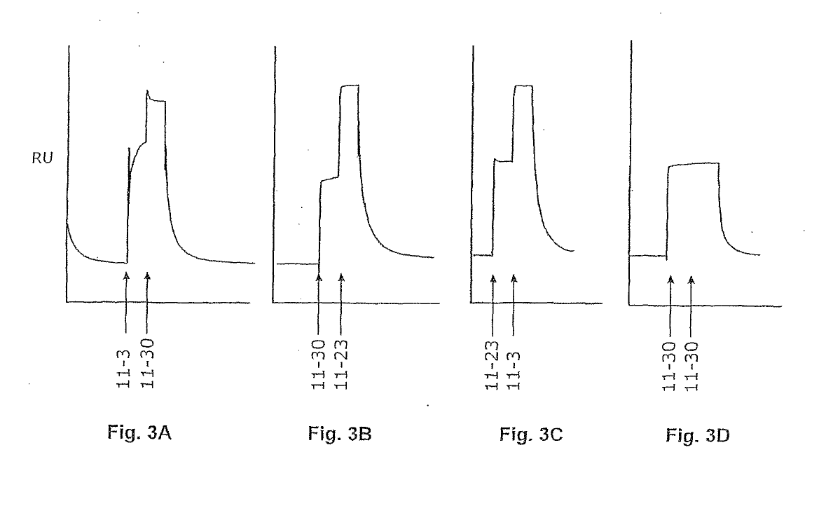

Further examples of suitable albumin, fragments and analogs for use in a ligand according to the invention are described in WO 03/076567A2, which is incorporated herein by reference in its entirety. In particular, the following albumin, fragments or variants can be used in the present invention:
Human serum albumin as described in WO 03/076567A2, e.g., in FIG. 3 (this sequence information being explicitly incorporated into the present disclosure by reference);
Human serum albumin (HA) consisting of a single non-glycosylated polypeptide chain of 585 amino acids with a formula molecular weight of 66,500 (See, Meloun, et al., FEBS Letters 58:136 (1975); Behrens, et al., Fed. Proc. 34:591 (1975); Lawn, et al., Nucleic Acids Research 9:6102-6114 (1981); Minghetti, et al., J. Biol. Chem. 261:6747 (1986));
A polymorphic variant or analog or fragment of albumin as described in Weitkamp, et al., Ann. Hum. Genet. 37:219 (1973);
An albumin fragment or variant as described in EP 322094, e.g., HA(1-373, HA(1-388), HA(1-389), HA(1-369), and HA(1-419) and fragments between 1-369 and 1-419;
An albumin fragment or variant as described in EP 399666, e.g., HA(1-177) and HA(1-200) and fragments between HA(1-X), where X is any number from 178 to 199.

Where a (one or more) half-life extending moiety (e.g., albumin, transferrin and fragments and analogues thereof) is used in the ligands of the invention, it can be conjugated to the ligand using any suitable method, such as, by direct fusion to the target-binding moiety (e.g., dAb or antibody fragment), for example by using a single nucleotide construct that encodes a fusion protein, wherein the fusion protein is encoded as a single polypeptide chain with the half-life extending moiety located N- or C-terminally to the cell surface target binding moieties. Alternatively, conjugation can be achieved by using a peptide linker between moieties, e.g., a peptide linker as described in WO 03/076567A2 or WO 2004/003019 (these linker disclosures being incorporated by reference in the present disclosure to provide examples for use in the present invention).

Typically, a polypeptide that enhances serum half-life in vivo is a polypeptide which occurs naturally in vivo and which resists degradation or removal by endogenous mechanisms which remove unwanted material from the organism (e.g., human). For example, a polypeptide that enhances serum half-life in vivo can be selected from proteins from the extracellular matrix, proteins found in blood, proteins found at the blood brain barrier or in neural tissue, proteins localized to the kidney, liver, lung, heart, skin or bone, stress proteins, disease-specific proteins, or proteins involved in Fc transport.

Suitable polypeptides that enhance serum half-life in vivo include, for example, transferrin receptor specific ligand-neuropharmaceutical agent fusion proteins (see U.S. Pat. No. 5,977,307, the teachings of which are incorporated herein by reference), brain capillary endothelial cell receptor, transferrin, transferrin receptor (e.g., soluble transferrin receptor), insulin, insulin-like growth factor 1 (IGF 1) receptor, insulin-like growth factor 2 (IGF 2) receptor, insulin receptor, blood coagulation factor X, α1-antitrypsin and HNF 1α. Suitable polypeptides that enhance serum half-life also include alpha-1 glycoprotein (orosomucoid; AAG), alpha-1 antichymotrypsin (ACT), alpha-1 microglobulin (protein HC; AIM), antithrombin III (AT III), apolipoprotein A-1 (Apo A-1), apolipoprotein B (Apo B), ceruloplasmin (Cp), complement component C3 (C3), complement component C4 (C4), C1 esterase inhibitor (C1 INH), C-reactive protein (CRP), ferritin (FER), hemopexin (HPX), lipoprotein(a) (Lp(a)), mannose-binding protein (MBP), myoglobin (Myo), prealbumin (transthyretin; PAL), retinol-binding protein (RBP), and rheumatoid factor (RF).

Suitable proteins from the extracellular matrix include, for example, collagens, laminins, integrins and fibronectin. Collagens are the major proteins of the extracellular matrix. About 15 types of collagen molecules are currently known, found in different parts of the body, e.g., type I collagen (accounting for 90% of body collagen) found in bone, skin, tendon, ligaments, cornea, internal organs or type II collagen found in cartilage, vertebral disc, notochord, and vitreous humor of the eye.

Suitable proteins from the blood include, for example, plasma proteins (e.g., fibrin, α-2 macroglobulin, serum albumin, fibrinogen (e.g., fibrinogen A, fibrinogen B), serum amyloid protein A, haptoglobin, profilin, ubiquitin, uteroglobulin and β-2-microglobulin), enzymes and enzyme inhibitors (e.g., plasminogen, lysozyme, cystatin C, alpha-1-antitrypsin and pancreatic trypsin inhibitor), proteins of the immune system, such as immunoglobulin proteins (e.g., IgA, IgD, IgE, IgG, IgM, immunoglobulin light chains (kappa/lambda)), transport proteins (e.g., retinol binding protein, α-1 microglobulin), defensins (e.g., beta-defensin 1, neutrophil defensin 1, neutrophil defensin 2 and neutrophil defensin 3) and the like.

Suitable proteins found at the blood brain barrier or in neural tissue include, for example, melanocortin receptor, myelin, ascorbate transporter and the like.

Suitable polypeptides that enhance serum half-life in vivo also include proteins localized to the kidney (e.g., polycystin, type IV collagen, organic anion transporter K1, Heymann's antigen), proteins localized to the liver (e.g., alcohol dehydrogenase, G250), proteins localized to the lung (e.g., secretory component, which binds IgA), proteins localized to the heart (e.g., HSP 27, which is associated with dilated cardiomyopathy), proteins localized to the skin (e.g., keratin), bone specific proteins such as morphogenic proteins (BMPs), which are a subset of the transforming growth factor β superfamily of proteins that demonstrate osteogenic activity (e.g., BMP-2, BMP-4, BMP-5, BMP-6, BMP-7, BMP-8), tumor specific proteins (e.g., trophoblast antigen, herceptin receptor, oestrogen receptor, cathepsins (e.g., cathepsin B, which can be found in liver and spleen)).

Suitable disease-specific proteins include, for example, antigens expressed only on activated T-cells, including LAG-3 (lymphocyte activation gene), osteoprotegerin ligand (OPGL; see Nature 402, 304-309 (1999)), OX40 (a member of the TNF receptor family, expressed on activated T cells and specifically up-regulated in human T cell leukemia virus type-I (HTLV-I)-producing cells; see Immunol. 165 (1):263-70 (2000)). Suitable disease-specific proteins also include, for example, metalloproteases (associated with arthritis/cancers) including CG6512 Drosophila, human paraplegin, human FtsH, human AFG3L2, murine ftsH; and angiogenic growth factors, including acidic fibroblast growth factor (FGF-1), basic fibroblast growth factor (FGF-2), vascular endothelial growth factor/vascular permeability factor (VEGF/VPF), transforming growth factor-α (TGF α), tumor necrosis factor-alpha (TNF-α), angiogenin, interleukin-3 (IL-3), interleukin-8 (IL-8), platelet-derived endothelial growth factor (PD-ECGF), placental growth factor (P1GF), midkine platelet-derived growth factor-BB (PDGF), and fractalkine.

Suitable polypeptides that enhance serum half-life in vivo also include stress proteins such as heat shock proteins (HSPs). HSPs are normally found intracellularly. When they are found extracellularly, it is an indicator that a cell has died and spilled out its contents. This unprogrammed cell death (necrosis) occurs when as a result of trauma, disease or injury, extracellular HSPs trigger a response from the immune system. Binding to extracellular HSP can result in localizing the compositions of the invention to a disease site.

Suitable proteins involved in Fc transport include, for example, Brambell receptor (also known as FcRB). This Fc receptor has two functions, both of which are potentially useful for delivery. The functions are (1) transport of IgG from mother to child across the placenta (2) protection of IgG from degradation thereby prolonging its serum half-life. It is thought that the receptor recycles IgG from endosomes. (See, Holliger et al., Nat Biotechnol 15(7):632-6 (1997).)

Methods for pharmacokinetic analysis and determination of ligand half-life will be familiar to those skilled in the art. Details may be found in Kenneth, A et al: Chemical Stability of Pharmaceuticals: A Handbook for Pharmacists and in Peters et al, Pharmacokinetc analysis: A Practical Approach (1996). Reference is also made to “Pharmacokinetics”, M Gibaldi & D Perron, published by Marcel Dekker, 2^{nd} Rev. ex edition (1982), which describes pharmacokinetic parameters such as t alpha and t beta half-lives and area under the curve (AUC).

Ligands that Contain a Toxin Moiety or Toxin

The invention also relates to ligands that comprise a toxin moiety or toxin. Suitable toxin moieties comprise a toxin (e.g., surface active toxin, cytotoxin). The toxin moiety or toxin can be linked or conjugated to the ligand using any suitable method. For example, the toxin moiety or toxin can be covalently bonded to the ligand directly or through a suitable linker. Suitable linkers can include noncleavable or cleavable linkers, for example, pH cleavable linkers that comprise a cleavage site for a cellular enzyme (e.g., cellular esterases, cellular proteases such as cathepsin B). Such cleavable linkers can be used to prepare a ligand that can release a toxin moiety or toxin after the ligand is internalized.

### Conjugation

A variety of methods for linking or conjugating a toxin moiety or toxin to a ligand can be used. The particular method selected will depend on the toxin moiety or toxin and ligand to be linked or conjugated. If desired, linkers that contain terminal functional groups can be used to link the ligand and toxin moiety or toxin. Generally, conjugation is accomplished by reacting toxin moiety or toxin that contains a reactive functional group (or is modified to contain a reactive functional group) with a linker or directly with a ligand. Covalent bonds can be formed by reacting a toxin moiety or toxin that contains (or is modified to contain) a chemical moiety or functional group that can, under appropriate conditions, react with a second chemical group thereby forming a covalent bond.

Many suitable reactive chemical group combinations are known in the art, for example, an amine group can react with an electrophilic group such as tosylate, mesylate, halo (chloro, bromo, fluoro, iodo), N-hydroxysuccinimidyl ester (NHS), and the like. Thiols can react with maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)).

The toxin conjugated ligand of the invention can be produced by reacting an appropriate ligand with a toxin comprising a reactive chemical or functional group, as described herein. For example, conjugation may be accomplished via primary amine residues, carboxy groups and cysteine residues. Engineered cysteine residues provide certain advantages as sites for toxin conjugation, because the conjugation of a toxin via an un-paired cysteine residue (e.g., a cysteine residue engineered into a ligand) provides a method to achieve site specific conjugation and reduces the likelihood that the conjugation will interfere with antigen binding function. For example, the unpaired cysteine can be incorporated at the carboxy-terminus of a dAb to provide a residue for site specific thiol conjugation. In addition, specific solvent accessible sites in the dual specific ligand which are not naturally occurring cysteine residues can be mutated to a cysteine for attachment of the toxin. Solvent accessible residues in the dual specific ligand can be determined using methods known in the art such as analysis of the crystal structures of a ligand. For example, using the solved crystal structure of the Vk dummy dAb (SEQ ID NO: 679), the residues Val-15, Pro-40, Gly-41, Ser-56, Gly-57, Ser-60, Pro-80, Glu-81, Gln-100, Lys-107 and Arg-108 have been identified as being solvent accessible, thus residues at corresponding positions on the dual specific ligands described herein are potential candidates for mutation to a cysteine residue for conjugation of the toxin.

Thiol conjugates can be prepared using any suitable method, such as the well-known methods for forming disulfide bonds or by reaction with a thiol reactive group such as maleimide, iodoacetyl, acrylolyl, pyridyl disulfides, 5-thiol-2-nitrobenzoic acid thiol (TNB-thiol), and the like.

In certain embodiments, a toxin or toxin moiety can be bonded to the ligand in a non-site specific manner by employing an amine-reactive chemical or functional group, for example, by reacting a ligand with an NHS ester of a toxin.

The preferred conjugation is a site specific conjugation, e.g., conjugation at a cysteine, amino terminus, or carboxy terminus. Amino-terminal conjugation can be accomplished using any suitable method, such as, the methods described in EP 0 822 199 B1. For example, a ligand can be reacted with an amine reactive toxin or toxin moiety under reducing alkylation conditions (e.g., in the presence of sodium borohydride, sodium cyanoborohyddride, dimethdylamine borate, trimethyl-amine borate or pyridine borate) at a pH suitable (e.g., 4.0-6.0) to selectively activate the α-amino group at the amino terminus of the ligand so that the toxin attaches to the α-amino, thus obtaining the ligand toxin conjugate.

Suitable toxin moieties and toxins include, for example, a maytansinoid (e.g., maytansinol, e.g., DM1, DM4), a taxane, a calicheamicin, a duocarmycin, or derivatives thereof. The maytansinoid can be, for example, maytansinol or a maytansinol analogue. Examples of maytansinol analogues include those having a modified aromatic ring (e.g., C-19-decloro, C-20-demethoxy, C-20-acyloxy) and those having modifications at other positions (e.g., C-9-CH, C-14-alkoxymethyl, C-14-hydroxymethyl or aceloxymethyl, C-15-hydroxy/acyloxy, C-15-methoxy, C-18-N-demethyl, 4,5-deoxy). Maytansinol and maytansinol analogues are described, for example, in U.S. Pat. Nos. 5,208,020 and 6,333,410, the contents of which is incorporated herein by reference. Maytansinol can be coupled to antibodies and antibody fragments using, e.g., an N-succinimidyl 3-(2-pyridyldithio)proprionate (also known as N-succinimidyl 4-(2-pyridyldithio)pentanoate or SPP), 4-succinimidyl-oxycarbonyl-a-(2-pyridyldithio)-toluene (SMPT), N-succinimidyl-3-(2-pyridyldithio)butyrate (SDPB), 2 iminothiolane, or S-acetylsuccinic anhydride. The taxane can be, for example, a taxol, taxotere, or novel taxane (see, e.g., WO 01/38318). The calicheamicin can be, for example, a bromo-complex calicheamicin (e.g., an alpha, beta or gamma bromo-complex), an iodo-complex calicheamicin (e.g., an alpha, beta or gamma iodo-complex), or analogs and mimics thereof. Bromo-complex calicheamicins include 11-BR, 12-BR, 13-BR, 14-BR, J1-BR, J2-BR and K1-BR. Iodo-complex calicheamicins include 11-1, 12-1,13-I, J1-I, J2-I, L1-I and K1-BR. Calicheamicin and mutants, analogs and mimics thereof are described, for example, in U.S. Pat. Nos. 4,970,198; 5,264,586; 5,550,246; 5,712,374, and 5,714,586, the contents of each of which are incorporated herein by reference. Duocarmycin analogs (e.g., KW-2189, DC88, DC89 CBI-TMI, and derivatives thereof are described, for example, in U.S. Pat. No. 5,070,092, U.S. Pat. No. 5,187,186, U.S. Pat. No. 5,641,780, U.S. Pat. No. 5,641,780, U.S. Pat. No. 4,923,990, and U.S. Pat. No. 5,101,038, the contents of each of which are incorporated herein by reference.

Examples of other toxins include, but are not limited to antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065 (see U.S. Pat. Nos. 5,475,092, 5,585,499, 5,846,545), melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, mitomycin, puromycin anthramycin (AMC)), duocarmycin and analogs or derivatives thereof, and anti-mitotic agents (e.g., vincristine, vinblastine, taxol, auristatins (e.g., auristatin E) and maytansinoids, and analogs or homologs thereof.

The toxin can also be a surface active toxin, such as a toxin that is a free radical generator (e.g., selenium containing toxin moieties), or radionuclide containing moiety. Suitable radionuclide containing moieties, include for example, moieties that contain radioactive iodine (¹³¹I or ¹²⁵I), yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), indium (¹¹¹In), technetium (⁹⁹mTc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga).

The toxin can be a protein, polypeptide or peptide, from bacterial sources, e.g., diphtheria toxin, pseudomonas exotoxin (PE) and plant proteins, e.g., the A chain of ricin (RTA), the ribosome inactivating proteins (RIPs) gelonin, pokeweed antiviral protein, saporin, and dodecandron are contemplated for use as toxins.

Antisense compounds of nucleic acids designed to bind, disable and promote degradation or prevent the production of the mRNA responsible for generating a particular target protein can also be used as a toxin. Antisense compounds include antisense RNA or DNA, single or double stranded, oligonucleotides, or their analogs, which can hybridize specifically to individual mRNA species and prevent transcription and/or RNA processing of the mRNA species and/or translation of the encoded polypeptide and thereby effect a reduction in the amount of the respective encoded polypeptide. Ching, et al., Proc. Natl. Acad. Sci. U.S.A. 86: 10006-10010 (1989); Broder, et al., Ann. Int. Med. 113: 604-618 (1990); Loreau, et al., FEBS Letters 274: 53-56 (1990); Useful antisense therapeutics include for example: Veglin™ (VasGene) and OGX-011 (Oncogenix).

Toxins can also be photoactive agents. Suitable photoactive agents include porphyrin-based materials such as porfimer sodium, the green porphyrins, chlorin E6, hematoporphyrin derivative itself, phthalocyanines, etiopurpurins, texaphrin, and the like.

The toxin can be an antibody or antibody fragment (e.g., intrabodies) that binds an intracellular target, such as a dAb that binds an intracellular target. Such antibodies or antibody fragments (dAbs) can be directed to defined subcellular compartments or targets. For example, the antibodies or antibody fragments (dAbs) can bind an intracellular target selected from erbB2, EGFR, BCR-ABL, p21Ras, Caspase3, Caspase7, Bcl-2, p53, Cyclin E, ATF-1/CREB, HPV16 E7, HP1, Type IV collagenases, cathepsin L as well as others described in Kontermann, R. E., Methods, 34:163-170 (2004), incorporated herein by reference in its entirety.

Polypeptide Domains that Bind CD38

The invention provides polypeptide domains (e.g., dAb) that have a binding site with binding specificity for CD38. In preferred embodiments, the polypeptide domain (e.g., dAb) binds to CD38 with low affinity. Preferably, the polypeptide domains binds CD38 with a K_{d} between about 10 μM to about 10 nM as determined by surface plasmon resonance. For example, the polypeptide domain can bind CD38 with an affinity of about 10 μM to about 300 nM, or about 10 μM to about 400 nM. In certain embodiments, the polypeptide domain binds CD38 with an affinity of about 300 nM to about 10 nM or 200 nM to about 10 nM.

In some embodiments, the polypeptide domain that has a binding site with binding specificity for CD38 competes for binding to CD38 with a dAb selected from the group consisting of: DOM11-14 (SEQ ID NO:39), DOM11-22 (SEQ ID NO: 40), DOM11-23 (SEQ ID NO: 32), DOM11-25 (SEQ ID NO: 41), DOM11-26 (SEQ ID NO: 42), DOM11-27 (SEQ ID NO: 43), DOM 11-29 (SEQ ID NO: 44), DOM11-3 (SEQ ID NO: 30), DOM11-30 (SEQ ID NO: 31), DOM11-31 (SEQ ID NO: 45), DOM11-32 (SEQ ID NO: 36), DOM11-36 (SEQ ID NO: 46), DOM11-4 (SEQ ID NO: 47), DOM11-43 (SEQ ID NO: 48), DOM11-44 (SEQ ID NO:49), DOM11-45 (SEQ ID NO: 50), DOM11-5 (SEQ ID NO: 51), DOM11-7 (SEQ ID NO: 33), DOM11-1 (SEQ ID NO: 52), DOM11-10 (SEQ ID NO: 53), DOM11-16 (SEQ ID NO:54), DOM11-2 (SEQ ID NO: 55), DOM11-20 (SEQ ID NO: 56), DOM11-21 (SEQ ID NO:57), DOM11-24 (SEQ ID NO:38), DOM11-28 (SEQ ID NO:58), DOM11-33 (SEQ ID NO: 59), DOM11-34 (SEQ ID NO: 60), DOM11-35 (SEQ ID NO:61), DOM11-37 (SEQ ID NO: 37), DOM11-38 (SEQ ID NO: 34), DOM11-39 (SEQ ID NO: 35), DOM11-41 (SEQ ID NO: 62), DOM11-42 (SEQ ID NO: 63), DOM11-6 (SEQ ID NO: 64), DOM11-8 (SEQ ID NO:65), and DOM11-9 (SEQ ID NO: 66).

In other embodiments, the polypeptide domain that has a binding site with binding specificity for CD38 competes for binding to CD38 with a dAb selected from the group consisting of: DOM 11-3-1 (SEQ ID NO: 269), DOM 11-3-2 (SEQ ID NO: 270), DOM 11-3-3 (SEQ ID NO: 271), DOM 11-3-4 (SEQ ID NO: 272), DOM 11-3-6 (SEQ ID NO: 273), DOM 11-3-9 (SEQ ID NO: 274), DOM 11-3-10 (SEQ ID NO: 275), DOM 11-3-11 (SEQ ID NO: 276), DOM 11-3-14 (SEQ ID NO: 277), DOM 11-3-15 (SEQ ID NO: 278), DOM 11-3-17 (SEQ ID NO: 279), DOM 11-3-19 (SEQ ID NO: 280), DOM 11-3-20 (SEQ ID NO: 281), DOM 11-3-21 (SEQ ID NO: 282), DOM 11-3-22 (SEQ ID NO: 283), DOM 11-3-23 (SEQ ID NO: 284), DOM 11-3-24 (SEQ ID NO: 285), DOM 11-3-25 (SEQ ID NO: 286), DOM 11-3-26 (SEQ ID NO: 287), DOM 11-3-27 (SEQ ID NO: 288), DOM 11-3-28 (SEQ ID NO: 289), DOM 11-30-1 (SEQ ID NO: 290), DOM 11-30-2 (SEQ ID NO: 291), DOM 11-30-3 (SEQ ID NO: 292), DOM 11-30-5 (SEQ ID NO: 293), DOM 11-30-6 (SEQ ID NO: 294), DOM 11-30-7 (SEQ ID NO: 295), DOM 11-30-8 (SEQ ID NO: 296), DOM 11-30-9 (SEQ ID NO: 297), DOM 11-30-10 (SEQ ID NO: 298), DOM 11-30-11 (SEQ ID NO: 299), DOM 11-30-12 (SEQ ID NO: 300), DOM 11-30-13 (SEQ ID NO: 301), DOM 11-30-14 (SEQ ID NO: 302), DOM 11-30-15 (SEQ ID NO: 303), DOM 11-30-16 (SEQ ID NO: 304), and DOM 11-30-17 (SEQ ID NO: 305).

In some embodiments, the polypeptide domain that has a binding site with binding specificity for CD38 comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence or a dAb selected from the group consisting of: DOM11-14 (SEQ ID NO:261), DOM11-22 (SEQ ID NO:262), DOM11-23 (SEQ ID NO:9), DOM11-25 (SEQ ID NO:263), DOM11-26 (SEQ ID NO:264), DOM11-27 (SEQ ID NO:265), DOM 11-29 (SEQ ID NO:266), DOM11-3 (SEQ ID NO:1), DOM11-30 (SEQ ID NO:2), DOM11-31 (SEQ ID NO:267), DOM11-32 (SEQ ID NO:7), DOM11-36 (SEQ ID NO:268), DOM11-4 (SEQ ID NO:269), DOM11-43 (SEQ ID NO:270), DOM11-44 (SEQ ID NO:271), DOM11-45 (SEQ ID NO:272), DOM11-5 (SEQ ID NO:273), DOM11-7 (SEQ ID NO:3), DOM11-1 (SEQ ID NO:274), DOM11-10 (SEQ ID NO:275), DOM11-16 (SEQ ID NO:276), DOM11-2 (SEQ ID NO:277), DOM11-20 (SEQ ID NO:278), DOM11-21 (SEQ ID NO:279), DOM11-24 (SEQ ID NO:6), DOM11-28 (SEQ ID NO:280), DOM11-33 (SEQ ID NO:281), DOM11-34 (SEQ ID NO:282), DOM11-35 (SEQ ID NO:283), DOM11-37 (SEQ ID NO:8), DOM1′-38 (SEQ ID NO:4), DOM11-39 (SEQ ID NO:5), DOM11-41 (SEQ ID NO:284), DOM11-42 (SEQ ID NO:285), DOM11-6 (SEQ ID NO:286), DOM11-8 (SEQ ID NO:287), and DOM11-9 (SEQ ID NO:288).

In other embodiments, the polypeptide domain that has a binding site with binding specificity for CD38 comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence or a dAb selected from the group consisting of: DOM 11-3-1 (SEQ ID NO: 269), DOM 11-3-2 (SEQ ID NO: 270), DOM 11-3-3 (SEQ ID NO: 271), DOM 11-3-4 (SEQ ID NO: 272), DOM 11-3-6 (SEQ ID NO: 273), DOM 11-3-9 (SEQ ID NO: 274), DOM 11-3-10 (SEQ ID NO: 275), DOM 11-3-11 (SEQ ID NO: 276), DOM 11-3-14 (SEQ ID NO: 277), DOM 11-3-15 (SEQ ID NO: 278), DOM 11-3-17 (SEQ ID NO: 279), DOM 11-3-19 (SEQ ID NO: 280), DOM 11-3-20 (SEQ ID NO: 281), DOM 11-3-21 (SEQ ID NO: 282), DOM 11-3-22 (SEQ ID NO: 283), DOM 11-3-23 (SEQ ID NO: 284), DOM 11-3-24 (SEQ ID NO: 285), DOM 11-3-25 (SEQ ID NO: 286), DOM 11-3-26 (SEQ ID NO: 287), DOM 11-3-27 (SEQ ID NO: 288), DOM 11-3-28 (SEQ ID NO: 289), DOM 11-30-1 (SEQ ID NO: 290), DOM 11-30-2 (SEQ ID NO: 291), DOM 11-30-3 (SEQ ID NO: 292), DOM 11-30-5 (SEQ ID NO: 293), DOM 11-30-6 (SEQ ID NO: 294), DOM 11-30-7 (SEQ ID NO: 295), DOM 11-30-8 (SEQ ID NO: 296), DOM 11-30-9 (SEQ ID NO: 297), DOM 11-30-10 (SEQ ID NO: 298), DOM 11-30-11 (SEQ ID NO: 299), DOM 11-30-12 (SEQ ID NO: 300), DOM 11-30-13 (SEQ ID NO: 301), DOM 11-30-14 (SEQ ID NO: 302), DOM 11-30-15 (SEQ ID NO: 303), DOM 11-30-16 (SEQ ID NO: 304), and DOM 11-30-17 (SEQ ID NO: 305).

In some embodiments, the polypeptide domain that has a binding site with binding specificity for CD38 competes with any of the dAbs disclosed herein for binding to CD38.

In preferred embodiments, the polypeptide domain that has a binding site with binding specificity for CD38 is selected from the group consisting of DOM11-3 (SEQ ID NO: 234), DOM11-30 (SEQ ID NO:254), DOM11-7 (SEQ ID NO:238), DOM11-38 (SEQ ID NO:262), DOM11-39 (SEQ ID NO:263), DOM11-24 (SEQ ID NO:248), DOM11-32 (SEQ ID NO:256), DOM11-37 (SEQ ID NO:261) and DOM11-23 (SEQ ID NO:247).

In other preferred embodiments, the polypeptide domain that has a binding site with binding specificity for CD38 is selected from the group consisting of DOM11-3-1 (SEQ ID NO:269), DOM11-3-2 (SEQ ID NO:270), DOM11-3-6 (SEQ ID NO:273), DOM11-3-10 (SEQ ID NO:275), DOM11-3-15 (SEQ ID NO:278), DOM11-3-20 (SEQ ID NO:281), DOM11-3-23 (SEQ ID NO:284), and DOM11-3-26 (SEQ ID NO:287).

In other preferred embodiments, the polypeptide domain that has a binding site with binding specificity for CD38 is selected from the group consisting of DOM11-30-1 (SEQ ID NO:290), DOM11-30-2 (SEQ ID NO:291), DOM1′-30-9 (SEQ ID NO:297), DOM11-3-15 (SEQ ID NO:303), and DOM11-30-16 (SEQ ID NO:304).

The polypeptide domain that has a binding site with binding specificity for CD38 can comprise any suitable immunoglobulin variable domain, and preferably comprises a human variable domain or a variable domain that comprises human framework regions. In certain embodiments, the polypeptide domain that has a binding site with binding specificity for CD38 comprises a universal framework, as described herein.

The universal framework can be a V_{L} framework (Vλ or Vκ), such as a framework that comprises the framework amino acid sequences encoded by the human germline DPK1, DPK2, DPK3, DPK4, DPK5, DPK6, DPK7, DPK8, DPK9, DPK10, DPK12, DPK13, DPK15, DPK16, DPK18, DPK19, DPK20, DPK21, DPK22, DPK23, DPK24, DPK25, DPK26 or DPK 28 immunoglobulin gene segment. If desired, the V_{L} framework can further comprise the framework amino acid sequence encoded by the human germline J_{κ}1, J_{κ}2, J_{κ}₃, J_{κ}₄, or J_{κ}₅ immunoglobulin gene segment.

In other embodiments the universal framework can be a V_{H} framework, such as a framework that comprises the framework amino acid sequences encoded by the human germline DP4, DP7, DP8, DP9, DP10, DP31, DP33, DP38, DP45, DP46, DP47, DP49, DP50, DP51, DP53, DP54, DP65, DP66, DP67, DP68 or DP69 immunoglobulin gene segment. If desired, the V_{H} framework can further comprise the framework amino acid sequence encoded by the human germline J_{H}1, J_{H}2, J_{H}3, J_{H}4, J_{H}4b, J_{H}5 and J_{H}6 immunoglobulin gene segment.

In certain embodiments, the polypeptide domain that has a binding site with binding specificity for CD38 comprises one or more framework regions comprising an amino acid sequence that is the same as the amino acid sequence of a corresponding framework region encoded by a human germline antibody gene segment, or the amino acid sequences of one or more of said framework regions collectively comprise up to 5 amino acid differences relative to the amino acid sequence of said corresponding framework region encoded by a human germline antibody gene segment.

In other embodiments, the amino acid sequences of FW1, FW2, FW3 and FW4 of the polypeptide domain that has a binding site with binding specificity for CD38 are the same as the amino acid sequences of corresponding framework regions encoded by a human germline antibody gene segment, or the amino acid sequences of FW1, FW2, FW3 and FW4 collectively contain up to 10 amino acid differences relative to the amino acid sequences of corresponding framework regions encoded by said human germline antibody gene segment.

In other embodiments, the polypeptide domain that has a binding site with binding specificity for CD38 comprises FW1, FW2 and FW3 regions, and the amino acid sequence of said FW1, FW2 and FW3 regions are the same as the amino acid sequences of corresponding framework regions encoded by human germline antibody gene segments.

In particular embodiments, the polypeptide domain that has a binding site with binding specificity for CD38 comprises the DPK9 V_{L} framework, or a V_{H} framework selected from the group consisting of DP47, DP45 and DP38. The polypeptide domain that has a binding site with binding specificity for CD38 can comprises a binding site for a generic ligand, such as protein A, protein L and protein G.

In certain embodiments, the polypeptide domain that has a binding site with binding specificity for CD38 is substantially resistant to aggregation. For example, in some embodiments, less than about 10%, less than about 9%, less than about 8%, less than about 7%, less than about 6%, less than about 5%, less than about 4%, less than about 3%, less than about 2% or less than about 1% of the polypeptide domain that has a binding site with binding specificity for CD38 aggregates when a 1-5 mg/ml, 5-10 mg/ml, 10-20 mg/ml, 20-50 mg/ml, 50-100 mg/ml, 100-200 mg/ml or 200-500 mg/ml solution of ligand or dAb in a solvent that is routinely used for drug formulation such as saline, buffered saline, citrate buffer saline, water, an emulsion, and, any of these solvents with an acceptable excipient such as those approved by the FDA, is maintained at about 22° C., 22-25° C., 25-30° C., 30-37° C., 37-40° C., 40-50° C., 50-60° C., 60-70° C., 70-80° C., 15-20° C., 10-15° C., 5-10° C., 2-5° C., 0-2° C., −10° C. to 0° C., −20° C. to −10° C., −40° C. to −20° C., −60° C. to −40° C., or −80° C. to −60° C., for a period of about time, for example, 10 minutes, 1 hour, 8 hours, 24 hours, 2 days, 3 days, 4 days, 1 week, 2 weeks, 3 weeks, 1 month, 2 months, 3 months, 4 months, 6 months, 1 year, or 2 years.

Aggregation can be assessed using any suitable method, such as, by microscopy, assessing turbidity of a solution by visual inspection or spectroscopy or any other suitable method. Preferably, aggregation is assessed by dynamic light scattering. Polypeptide domains that have a binding site with binding specificity for CD38 that are resistant to aggregation provide several advantages. For example, such polypeptide domains that have a binding site with binding specificity for CD38 can readily be produced in high yield as soluble proteins by expression using a suitable biological production system, such as E. coli, and can be formulated and/or stored at higher concentrations than conventional polypeptides, and with less aggregation and loss of activity.

In addition, the polypeptide domain that has a binding site with binding specificity for CD38 that are resistant to aggregation can be produced more economically than other antigen- or epitope-binding polypeptides (e.g., conventional antibodies). For example, generally, preparation of antigen- or epitope-binding polypeptides intended for in vivo applications includes processes (e.g., gel filtration) that remove aggregated polypeptides. Failure to remove such aggregates can result in a preparation that is not suitable for in vivo applications because, for example, aggregates of an antigen-binding polypeptide that is intended to act as an antagonist can function as an agonist by inducing cross-linking or clustering of the target antigen. Protein aggregates can also reduce the efficacy of therapeutic polypeptide by inducing an immune response in the subject to which they are administered.

In contrast, the aggregation resistant polypeptide domain that has a binding site with binding specificity for CD38 of the invention can be prepared for in vivo applications without the need to include process steps that remove aggregates, and can be used in in vivo applications without the aforementioned disadvantages caused by polypeptide aggregates.

In some embodiments, a polypeptide domain that has a binding site with binding specificity for CD38 unfolds reversibly when heated to a temperature (Ts) and cooled to a temperature (Tc), wherein Ts is greater than the melting temperature (Tm) of the polypeptide domain that has a binding site with binding specificity for CD38, and Tc is lower than the melting temperature of the polypeptide domain that has a binding site with binding specificity for CD38. For example, polypeptide domain that has a binding site with binding specificity for CD38 can unfold reversibly when heated to 80° C. and cooled to about room temperature. A polypeptide that unfolds reversibly loses function when unfolded but regains function upon refolding. Such polypeptides are distinguished from polypeptides that aggregate when unfolded or that improperly refold (misfolded polypeptides), i.e., do not regain function.

Polypeptide unfolding and refolding can be assessed, for example, by directly or indirectly detecting polypeptide structure using any suitable method. For example, polypeptide structure can be detected by circular dichroism (CD) (e.g., far-UV CD, near-UV CD), fluorescence (e.g., fluorescence of tryptophan side chains), susceptibility to proteolysis, nuclear magnetic resonance (NMR), or by detecting or measuring a polypeptide function that is dependent upon proper folding (e.g., binding to target ligand, binding to generic ligand). In one example, polypeptide unfolding is assessed using a functional assay in which loss of binding function (e.g., binding a generic and/or target ligand, binding a substrate) indicates that the polypeptide is unfolded.

The extent of unfolding and refolding of a polypeptide domain that has a binding site with binding specificity for CD38 can be determined using an unfolding or denaturation curve. An unfolding curve can be produced by plotting temperature as the ordinate and the relative concentration of folded polypeptide as the abscissa. The relative concentration of a folded polypeptide domain that has a binding site with binding specificity for CD38 can be determined directly or indirectly using any suitable method (e.g., CD, fluorescence, binding assay). For example, a polypeptide domain that has a binding site with binding specificity for CD38 solution can be prepared and ellipticity of the solution determined by CD. The ellipticity value obtained represents a relative concentration of folded ligand or dAb monomer of 100%. The polypeptide domain that has a binding site with binding specificity for CD38 in the solution is then unfolded by incrementally raising the temperature of the solution and ellipticity is determined at suitable increments (e.g., after each increase of one degree in temperature). The polypeptide domain that has a binding site with binding specificity for CD38 in solution is then refolded by incrementally reducing the temperature of the solution and ellipticity is determined at suitable increments. The data can be plotted to produce an unfolding curve and a refolding curve. The unfolding and refolding curves have a characteristic sigmoidal shape that includes a portion in which the polypeptide domain that has a binding site with binding specificity for CD38 molecules are folded, an unfolding/refolding transition in which polypeptide domain that has a binding site with binding specificity for CD38 molecules are unfolded to various degrees, and a portion in which polypeptide domain that has a binding site with binding specificity for CD38 are unfolded. The y-axis intercept of the refolding curve is the relative amount of refolded polypeptide domain that has a binding site with binding specificity for CD38 recovered. A recovery of at least about 50%, or at least about 60%, or at least about 70%, or at least about 75%, or at least about 80%, or at least about 85%, or at least about 90%, or at least about 95% is indicative that the ligand or dAb monomer unfolds reversibly.

In a preferred embodiment, reversibility of unfolding of a polypeptide domain that has a binding site with binding specificity for CD38 is determined by preparing a polypeptide domain that has a binding site with binding specificity for CD38 solution and plotting heat unfolding and refolding curves. The polypeptide domain that has a binding site with binding specificity for CD38 solution can be prepared in any suitable solvent, such as an aqueous buffer that has a pH suitable to allow a polypeptide domain that has a binding site with binding specificity for CD38 to dissolve (e.g., pH that is about 3 units above or below the isoelectric point (pI)). The polypeptide domain that has a binding site with binding specificity for CD38 solution is concentrated enough to allow unfolding/folding to be detected. For example, the ligand or dAb monomer solution can be about 0.1 μM to about 100 μM, or preferably about 1 μM to about 10 μM.

If the melting temperature (Tm) of polypeptide domain that has a binding site with binding specificity for CD38 is known, the solution can be heated to about ten degrees below the Tm (Tm-10) and folding assessed by ellipticity or fluorescence (e.g., far-UV CD scan from 200 nm to 250 nm, fixed wavelength CD at 235 nm or 225 nm; tryptophan fluorescent emission spectra at 300 to 450 nm with excitation at 298 nm) to provide 100% relative folded ligand or dAb monomer. The solution is then heated to at least ten degrees above Tm (Tm+10) in predetermined increments (e.g., increases of about 0.1 to about 1 degree), and ellipticity or fluorescence is determined at each increment. Then, the polypeptide domain that has a binding site with binding specificity for CD38 is refolded by cooling to at least Tm-10 in predetermined increments and ellipticity or fluorescence determined at each increment. If the melting temperature of a polypeptide domain that has a binding site with binding specificity for CD38 is not known, the solution can be unfolded by incrementally heating from about 25° C. to about 100° C. and then refolded by incrementally cooling to at least about 25° C., and ellipticity or fluorescence at each heating and cooling increment is determined. The data obtained can be plotted to produce an unfolding curve and a refolding curve, in which the y-axis intercept of the refolding curve is the relative amount of refolded protein recovered. In some embodiments, the polypeptide domain that has a binding site with binding specificity for CD38 does not comprise a Camelid immunoglobulin variable domain, or one or more framework amino acids that are unique to immunoglobulin variable domains encoded by Camelid germline antibody gene segments.

Preferably, the polypeptide domain that has a binding site with binding specificity for CD38 is secreted in a quantity of at least about 0.5 mg/L when expressed in E. coli or in Pichia species (e.g., P. pastoris). In other preferred embodiments, a polypeptide domain that has a binding site with binding specificity for CD38 is secreted in a quantity of at least about 0.75 mg/L, at least about 1 mg/L, at least about 4 mg/L, at least about 5 mg/L, at least about 10 mg/L, at least about 15 mg/L, at least about 20 mg/L, at least about 25 mg/L, at least about 30 mg/L, at least about 35 mg/L, at least about 40 mg/L, at least about 45 mg/L, or at least about 50 mg/L, or at least about 100 mg/L, or at least about 200 mg/L, or at least about 300 mg/L, or at least about 400 mg/L, or at least about 500 mg/L, or at least about 600 mg/L, or at least about 700 mg/L, or at least about 800 mg/L, at least about 900 mg/L, or at least about 1 g/L when expressed in E. coli or in Pichia species (e.g., P. pastoris). In other preferred embodiments, a polypeptide domain that has a binding site with binding specificity for CD38 is secreted in a quantity of at least about 1 mg/L to at least about 1 g/L, at least about 1 mg/L to at least about 750 mg/L, at least about 100 mg/L to at least about 1 g/L, at least about 200 mg/L to at least about 1 g/L, at least about 300 mg/L to at least about 1 g/L, at least about 400 mg/L to at least about 1 g/L, at least about 500 mg/L to at least about 1 g/L, at least about 600 mg/L to at least about 1 g/L, at least about 700 mg/L to at least about 1 g/L, at least about 800 mg/L to at least about 1 g/L, or at least about 900 mg/L to at least about 1 g/L when expressed in E. coli or in Pichia species (e.g., P. pastoris). Although, a polypeptide domain that has a binding site with binding specificity for CD38 described herein can be secretable when expressed in E. coli or in Pichia species (e.g., P. pastoris), it can be produced using any suitable method, such as synthetic chemical methods or biological production methods that do not employ E. coli or Pichia species.

Polypeptide Domains that Bind CD138

The invention provides polypeptide domains (e.g., dAb) that have a binding site with binding specificity for CD138. In preferred embodiments, the polypeptide domain binds to CD138 with low affinity. Preferably, the polypeptide domain binds CEA with a K_{d} between about 10 μM to about 10 nM as determined by surface plasmon resonance. For example, the polypeptide domain can bind CD138 with an affinity of about 10 μM to about 300 nM, or about 10 μM to about 400 dM. In certain embodiments, the polypeptide domain binds CD138 with an affinity of about 300 nM to about 10 nM or 200 nM to about 10 nM.

In some embodiments, the a polypeptide domain that has a binding site with binding specificity for CD138 competes for binding to CD138 with a dAb selected from the group consisting of: DOM12-1 (SEQ ID NO: 70), DOM12-15 (SEQ ID NO: 71), DOM12-17 (SEQ ID NO: 68), DOM12-19 (SEQ ID NO: 72), DOM12-2 (SEQ ID NO: 73), DOM12-20 (SEQ ID NO: 74), DOM12-21 (SEQ ID NO: 75), DOM12-22 (SEQ ID NO: 76), DOM12-3 (SEQ ID NO: 77), DOM12-33 (SEQ ID NO:78), DOM12-39 (SEQ ID NO: 79), DOM12-4 (SEQ ID NO: 80), DOM12-40 (SEQ ID NO: 81), DOM12-41 (SEQ ID NO: 82), DOM12-42 (SEQ ID NO:83), DOM12-44 (SEQ ID NO: 84), DOM12-46 (SEQ ID NO: 85), DOM12-6 (SEQ ID NO: 86), DOM12-7 (SEQ ID NO: 87), DOM12-10 (SEQ ID NO:88), DOM12-11 (SEQ ID NO:89), DOM12-18 (SEQ ID NO: 90), DOM12-23 (SEQ ID NO: 91), DOM12-24 (SEQ ID NO: 92), DOM12-25 (SEQ ID NO: 93), DOM12-26 (SEQ ID NO:69), DOM12-27 (SEQ ID NO: 94), DOM12-28 (SEQ ID NO:95), DOM12-29 (SEQ ID NO: 96), DOM12-30 (SEQ ID NO: 97), DOM12-31 (SEQ ID NO:98), DOM12-32 (SEQ ID NO: 99), DOM12-34 (SEQ ID NO:100), DOM12-35 (SEQ ID NO: 101), DOM12-36 (SEQ ID NO:102), DOM12-37 (SEQ ID NO:103), DOM12-38 (SEQ ID NO:104), DOM12-43 (SEQ ID NO:105), DOM12-45 (SEQ ID NO: 67), DOM12-5 (SEQ ID NO: 106), DOM12-8 (SEQ ID NO: 107), and DOM12-9 (SEQ ID NO: 108).

In some embodiments, the a polypeptide domain that has a binding site with binding specificity for CD138 competes for binding to CD138 with a dAb selected from the group consisting of: DOM 12-45-1 (SEQ ID NO: 348), DOM 12-45-2 (SEQ ID NO: 349), DOM 12-45-3 (SEQ ID NO: 350), DOM 12-45-4 (SEQ ID NO: 351), DOM 12-45-5 (SEQ ID NO: 352), DOM 12-45-6 (SEQ ID NO: 353), DOM 12-45-8 (SEQ ID NO: 354), DOM 12-45-9 (SEQ ID NO: 355), DOM 12-45-10 (SEQ ID NO: 356), DOM 12-45-11 (SEQ ID NO: 357), DOM 12-45-12 (SEQ ID NO: 358), DOM 12-45-13 (SEQ ID NO: 359), DOM 12-45-14 (SEQ ID NO: 360), DOM 12-45-15 (SEQ ID NO: 361), DOM 12-45-16 (SEQ ID NO: 362), DOM 12-45-17 (SEQ ID NO: 363), DOM 12-45-18 (SEQ ID NO: 364), DOM 12-45-19 (SEQ ID NO: 365), DOM 12-45-20 (SEQ ID NO: 366), DOM 12-45-21 (SEQ ID NO: 367), DOM 12-45-22 (SEQ ID NO: 368), DOM 12-45-23 (SEQ ID NO: 369), DOM 12-45-24 (SEQ ID NO: 370), DOM 12-45-25 (SEQ ID NO: 371), DOM 12-45-26 (SEQ ID NO: 372), DOM 12-45-27 (SEQ ID NO: 373), DOM 12-45-28 (SEQ ID NO: 374), DOM 12-45-29 (SEQ ID NO: 375), DOM 12-45-30 (SEQ ID NO: 376), DOM 12-45-31 (SEQ ID NO: 377), DOM 12-45-32 (SEQ ID NO: 378), DOM 12-45-33 (SEQ ID NO: 379), DOM 12-45-34 (SEQ ID NO: 380), DOM 12-45-35 (SEQ ID NO: 381), DOM 12-45-36 (SEQ ID NO: 382), DOM 12-45-37 (SEQ ID NO: 383), and DOM 12-45-38 (SEQ ID NO: 384).

In some embodiments, the polypeptide domain that has a binding site with binding specificity for CD138 comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence of a dAb selected from the group consisting of: DOM12-1 (SEQ ID NO:289), DOM12-15 (SEQ ID NO:290), DOM12-17 (SEQ ID NO:11), DOM12-19 (SEQ ID NO:291), DOM12-2 (SEQ ID NO:292), DOM12-20 (SEQ ID NO:293), DOM12-21 (SEQ ID NO:294), DOM12-22 (SEQ ID NO:295), DOM12-3 (SEQ ID NO:296), DOM12-33 (SEQ ID NO:297), DOM12-39 (SEQ ID NO:298), DOM12-4 (SEQ ID NO:299), DOM12-40 (SEQ ID NO:300), DOM12-41 (SEQ ID NO:301), DOM12-42 (SEQ ID NO:302), DOM12-44 (SEQ ID NO:303), DOM12-46 (SEQ ID NO:304), DOM12-6 (SEQ ID NO:305), DOM12-7 (SEQ ID NO:306), DOM12-10 (SEQ ID NO:307), DOM12-11 (SEQ ID NO:308), DOM12-18 (SEQ ID NO:309), DOM12-23 (SEQ ID NO:310), DOM12-24 (SEQ ID NO:311), DOM12-25 (SEQ ID NO:312), DOM12-26 (SEQ ID NO:12), DOM12-27 (SEQ ID NO:313), DOM12-28 (SEQ ID NO:314), DOM12-29 (SEQ ID NO:315), DOM12-30 (SEQ ID NO:316), DOM12-31 (SEQ ID NO:317), DOM12-32 (SEQ ID NO:318), DOM12-34 (SEQ ID NO:319), DOM12-35 (SEQ ID NO:320), DOM12-36 (SEQ ID NO:321), DOM12-37 (SEQ ID NO:322), DOM12-38 (SEQ ID NO:323), DOM12-43 (SEQ ID NO:324), DOM12-45 (SEQ ID NO:310), DOM12-5 (SEQ ID NO:325), DOM12-8 (SEQ ID NO:326), and DOM12-9 (SEQ ID NO:327).

In some embodiments, the polypeptide domain that has a binding site with binding specificity for CD138 comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence of a dAb selected from the group consisting of: DOM 12-45-1 (SEQ ID NO: 348), DOM 12-45-2 (SEQ ID NO: 349), DOM 12-45-3 (SEQ ID NO: 350), DOM 12-45-4 (SEQ ID NO: 351), DOM 12-45-5 (SEQ ID NO: 352), DOM 12-45-6 (SEQ ID NO: 353), DOM 12-45-8 (SEQ ID NO: 354), DOM 12-45-9 (SEQ ID NO: 355), DOM 12-45-10 (SEQ ID NO: 356), DOM 12-45-11 (SEQ ID NO: 357), DOM 12-45-12 (SEQ ID NO: 358), DOM 12-45-13 (SEQ ID NO: 359), DOM 12-45-14 (SEQ ID NO: 360), DOM 12-45-15 (SEQ ID NO: 361), DOM 12-45-16 (SEQ ID NO: 362), DOM 12-45-17 (SEQ ID NO: 363), DOM 12-45-18 (SEQ ID NO: 364), DOM 12-45-19 (SEQ ID NO: 365), DOM 12-45-20 (SEQ ID NO: 366), DOM 12-45-21 (SEQ ID NO: 367), DOM 12-45-22 (SEQ ID NO: 368), DOM 12-45-23 (SEQ ID NO: 369), DOM 12-45-24 (SEQ ID NO: 370), DOM 12-45-25 (SEQ ID NO: 371), DOM 12-45-26 (SEQ ID NO: 372), DOM 12-45-27 (SEQ ID NO: 373), DOM 12-45-28 (SEQ ID NO: 374), DOM 12-45-29 (SEQ ID NO: 375), DOM 12-45-30 (SEQ ID NO: 376), DOM 12-45-31 (SEQ ID NO: 377), DOM 12-45-32 (SEQ ID NO: 378), DOM 12-45-33 (SEQ ID NO: 379), DOM 12-45-34 (SEQ ID NO: 380), DOM 12-45-35 (SEQ ID NO: 381), DOM 12-45-36 (SEQ ID NO: 382), DOM 12-45-37 (SEQ ID NO: 383), and DOM 12-45-38 (SEQ ID NO: 384).

In some embodiments, the polypeptide domain that has a binding site with binding specificity for CD138 competes with any of the dAbs disclosed herein for binding to CD138.

In preferred embodiments, the polypeptide domain that has a binding site with binding specificity for CD38 is selected from the group consisting of DOM 12-45 (SEQ ID NO: 346), DOM12-17 (SEQ ID NO: 318) and DOM 12-26 (SEQ ID NO: 327).

In other preferred embodiments, the polypeptide domain that has a binding site with binding specificity for CD38 is selected from the group consisting of DOM 12-45-1 (SEQ ID NO:348), DOM12-45-2 (SEQ ID NO:349) and DOM 12-45-5 (SEQ ID NO:352).

The polypeptide domain that has a binding site with binding specificity for CD138 can comprise any suitable immunoglobulin variable domain, and preferably comprises a human variable domain or a variable domain that comprises human framework regions. In certain embodiments, the polypeptide domain that has a binding site with binding specificity for CD138 comprises a universal framework, as described herein.

In certain embodiments, the polypeptide domain that has a binding site with binding specificity for CD138 resists aggregation, unfolds reversibly and/or comprises a framework region and is secreted as described above for the polypeptide domain that has a binding site with binding specificity for CD38. Polypeptide Domains that Bind CEA.

The invention provides polypeptide domains (e.g., dAb) that have a binding site with binding specificity for CEA. In preferred embodiments, the polypeptide domain binds to CEA with low affinity. Preferably, the polypeptide domain binds CEA with a K_{d} between about 10 μM to about 10 nM as determined by surface plasmon resonance. For example, the polypeptide domain can bind CEA with an affinity of about 10 μM to about 300 nM, or about 10 μM to about 400 nM. In certain embodiments, the polypeptide domain binds CEA with an affinity of about 300 nM to about 10 nM or 200 nM to about 10 nM.

In some embodiments, the polypeptide domain that has a binding site with binding specificity for CEA competes for binding to CEA with a dAb selected from the group consisting of DOM13-1 (SEQ ID NO:385), DOM13-12 (SEQ ID NO:393), DOM13-13 (SEQ ID NO:394), DOM13-14 (SEQ ID NO:395), DOM13-15 (SEQ ID NO:3396), DOM13-16 (SEQ ID NO:397), DOM13-17 (SEQ ID NO:398), DOM13-18 (SEQ ID NO:399), DOM13-19 (SEQ ID NO:400), DOM13-2 (SEQ ID NO:386), DOM13-20 (SEQ ID NO:401), DOM13-21 (SEQ ID NO:402), DOM13-22 (SEQ ID NO:403), DOM13-23 (SEQ ID NO:404), DOM13-24 (SEQ ID NO:3405), DOM13-25 (SEQ ID NO:406), DOM13-26 (SEQ ID NO:407), DOM13-27 (SEQ ID NO:408), DOM13-28 (SEQ ID NO:409), DOM13-29 (SEQ ID NO:410), DOM13-3 (SEQ ID NO:387), DOM13-30 (SEQ ID NO:411), DOM13-31 (SEQ ID NO:412), DOM13-32 (SEQ ID NO:413), DOM13-33 (SEQ ID NO:414), DOM-13-34 (SEQ ID NO:415), DOM13-35 (SEQ ID NO:416), DOM13-36 (SEQ ID NO:417), DOM13-37 (SEQ ID NO:418), DOM13-4 (SEQ ID NO:388), DOM13-42 (SEQ ID NO:419), DOM13-43 (SEQ ID NO:420), DOM13-44 (SEQ ID NO:421), DOM13-45 (SEQ ID NO:422), DOM13-46 (SEQ ID NO:423), DOM13-47 (SEQ ID NO:424), DOM13-48 (SEQ ID NO:425), DOM13-49 (SEQ ID NO:426), DOM13-5 (SEQ ID NO:389), DOM13-50 (SEQ ID NO:427), DOM13-51 (SEQ ID NO:428), DOM13-52 (SEQ ID NO:429), DOM13-53 (SEQ ID NO:430), DOM13-54 (SEQ ID NO:431), DOM13-55 (SEQ ID NO:432), DOM13-56 (SEQ ID NO:433), DOM13-57 (SEQ ID NO:434), DOM13-58 (SEQ ID NO:435), DOM13-59 (SEQ ID NO:436), DOM13-6 (SEQ ID NO:390), DOM13-60 (SEQ ID NO:437), DOM13-61 (SEQ ID NO:438), DOM13-62 (SEQ ID NO:439), DOM13-63 (SEQ ID NO:440), DOM13-64 (SEQ ID NO:441), DOM13-65 (SEQ ID NO:442), DOM13-66 (SEQ ID NO:443), DOM13-67 (SEQ ID NO:444), DOM13-68 (SEQ ID NO:445), DOM13-69 (SEQ ID NO:446), DOM13-7 (SEQ ID NO:391), DOM13-70 (SEQ ID NO:447), DOM13-71 (SEQ ID NO:3448), DOM13-72 (SEQ ID NO:449), DOM13-73 (SEQ ID NO:450), DOM13-74 (SEQ ID NO:451), DOM13-75 (SEQ ID NO:452), DOM13-76 (SEQ ID NO:453), DOM13-77 (SEQ ID NO:454), DOM13-78 (SEQ ID NO:455), DOM13-79 (SEQ ID NO:456), DOM13-8 (SEQ ID NO:392), DOM13-80 (SEQ ID NO:457), DOM13-81 (SEQ ID NO:458), DOM13-82 (SEQ ID NO:459), DOM13-83 (SEQ ID NO:460), DOM13-84 (SEQ ID NO:461), DOM13-85 (SEQ ID NO:462), DOM13-86 (SEQ ID NO:463), DOM13-87 (SEQ ID NO:464), DOM13-88 (SEQ ID NO:465), DOM13-89 (SEQ ID NO:466), DOM13-90 (SEQ ID NO:467), DOM13-91 (SEQ ID NO:468), DOM13-92 (SEQ ID NO:469), DOM13-93 (SEQ ID NO:470), DOM13-94 (SEQ ID NO:471), and DOM13-95 (SEQ ID NO:472).

In certain embodiments, the polypeptide domain that has a binding site with binding specificity for CEA competes for binding to CEA with a dAb selected from the group consisting of DOM 13-25-3 (SEQ ID NO: 473), DOM 13-25-23 (SEQ ID NO: 474), DOM 13-25-27 (SEQ ID NO: 475), and DOM 13-25-80 (SEQ ID NO: 476).

In some embodiments, the polypeptide domain that has a binding site with binding specificity for CEA comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence or a dAb selected from the group consisting of: DOM13-1 (SEQ ID NO:385), DOM13-12 (SEQ ID NO:393), DOM13-13 (SEQ ID NO:394), DOM13-14 (SEQ ID NO:395), DOM13-15 (SEQ ID NO:3396), DOM13-16 (SEQ ID NO:397), DOM13-17 (SEQ ID NO:398), DOM13-18 (SEQ ID NO:399), DOM13-19 (SEQ ID NO:400), DOM13-2 (SEQ ID NO:386), DOM13-20 (SEQ ID NO:401), DOM13-21 (SEQ ID NO:402), DOM13-22 (SEQ ID NO:403), DOM13-23 (SEQ ID NO:404), DOM13-24 (SEQ ID NO:3405), DOM13-25 (SEQ ID NO:406), DOM13-26 (SEQ ID NO:407), DOM13-27 (SEQ ID NO:408), DOM13-28 (SEQ ID NO:409), DOM13-29 (SEQ ID NO:410), DOM13-3 (SEQ ID NO:387), DOM13-30 (SEQ ID NO:411), DOM13-31 (SEQ ID NO:412), DOM13-32 (SEQ ID NO:413), DOM13-33 (SEQ ID NO:414), DOM-13-34 (SEQ ID NO:415), DOM13-35 (SEQ ID NO:416), DOM13-36 (SEQ ID NO:417), DOM13-37 (SEQ ID NO:418), DOM13-4 (SEQ ID NO:388), DOM13-42 (SEQ ID NO:419), DOM13-43 (SEQ ID NO:420), DOM13-44 (SEQ ID NO:421), DOM13-45 (SEQ ID NO:422), DOM13-46 (SEQ ID NO:423), DOM13-47 (SEQ ID NO:424), DOM13-48 (SEQ ID NO:425), DOM13-49 (SEQ ID NO:426), DOM13-5 (SEQ ID NO:389), DOM13-50 (SEQ ID NO:427), DOM13-51 (SEQ ID NO:428), DOM13-52 (SEQ ID NO:429), DOM13-53 (SEQ ID NO:430), DOM13-54 (SEQ ID NO:431), DOM13-55 (SEQ ID NO:432), DOM13-56 (SEQ ID NO:433), DOM13-57 (SEQ ID NO:434), DOM13-58 (SEQ ID NO:435), DOM13-59 (SEQ ID NO:436), DOM13-6 (SEQ ID NO:390), DOM13-60 (SEQ ID NO:437), DOM13-61 (SEQ ID NO:438), DOM13-62 (SEQ ID NO:439), DOM13-63 (SEQ ID NO:440), DOM13-64 (SEQ ID NO:441), DOM13-65 (SEQ ID NO:442), DOM13-66 (SEQ ID NO:443), DOM13-67 (SEQ ID NO:444), DOM13-68 (SEQ ID NO:445), DOM13-69 (SEQ ID NO:446), DOM13-7 (SEQ ID NO:391), DOM13-70 (SEQ ID NO:447), DOM13-71 (SEQ ID NO:3448), DOM13-72 (SEQ ID NO:449), DOM13-73 (SEQ ID NO:450), DOM13-74 (SEQ ID NO:451), DOM13-75 (SEQ ID NO:452), DOM13-76 (SEQ ID NO:453), DOM13-77 (SEQ ID NO:454), DOM13-78 (SEQ ID NO:455), DOM13-79 (SEQ ID NO:456), DOM13-8 (SEQ ID NO:392), DOM13-80 (SEQ ID NO:457), DOM13-81 (SEQ ID NO:458), DOM13-82 (SEQ ID NO:459), DOM13-83 (SEQ ID NO:460), DOM13-84 (SEQ ID NO:461), DOM13-85 (SEQ ID NO:462), DOM13-86 (SEQ ID NO:463), DOM13-87 (SEQ ID NO:464), DOM13-88 (SEQ ID NO:465), DOM13-89 (SEQ ID NO:466), DOM13-90 (SEQ ID NO:467), DOM13-91 (SEQ ID NO:468), DOM13-92 (SEQ ID NO:469), DOM13-93 (SEQ ID NO:470), DOM13-94 (SEQ ID NO:471), and DOM13-95 (SEQ ID NO:472).

In other embodiments, the polypeptide domain that has a binding site with binding specificity for CEA comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence or a dAb selected from the group consisting of: DOM 13-25-3 (SEQ ID NO: 473), DOM 13-25-23 (SEQ ID NO: 474), DOM 13-25-27 (SEQ ID NO: 475), and DOM 13-25-80 (SEQ ID NO: 476).

In preferred embodiments, the polypeptide domain that has a binding site with binding specificity for CEA is selected from the group consisting of: DOM13-25 (SEQ ID NO: 80), DOM13-57 (SEQ ID NO: 81), DOM13-58 (SEQ ID NO:82), DOM13-59 (SEQ ID NO:83), DOM13-64 (SEQ ID NO:84), DOM13-65 (SEQ ID NO:85), DOM13-74 (SEQ ID NO:86), DOM13-93 (SEQ ID NO:87), and DOM13-95 (SEQ ID NO:88). In some embodiments, the polypeptide domain that has a binding site with binding specificity for CEA competes with any of the dAbs disclosed herein for binding to CEA.

The polypeptide domain that has a binding site with binding specificity for CEA can comprise any suitable immunoglobulin variable domain, and preferably comprises a human variable domain or a variable domain that comprises human framework regions. In certain embodiments, the polypeptide domain that has a binding site with binding specificity for CEA comprises a universal framework, as described herein.

In certain embodiments, the polypeptide domain that has a binding site with binding specificity for CEA resists aggregation, unfolds reversibly and/or comprises a framework region and is secreted, as described above for the polypeptide domain that has a binding site with binding specificity for CD38.

Polypeptide Domains that Bind CD56

The invention provides polypeptide domains (e.g., dAb) that have a binding site with binding specificity for CD56. In preferred embodiments, the polypeptide domain binds to CD56 with low affinity. Preferably, the polypeptide domain binds CD56 with a K_{d} between about 10 μM to about 10 nM as determined by surface plasmon resonance. For example, the polypeptide domain can bind CD56 with an affinity of about 10 μM to about 300 nM, or about 10 μM to about 400 nM. In certain embodiments, the polypeptide domain binds CD56 with an affinity of about 300 nM to about 10 nM or 200 nM to about 10 nM.

In some embodiments, the polypeptide domain that has a binding site with binding specificity for CD56 competes for binding to CD56 with a dAb selected from the group consisting of DOM14-1 (SEQ ID NO:477), DOM14-10 (SEQ ID NO:481), DOM14-100 (SEQ ID NO:540), DOM14-11 (SEQ ID NO:482), DOM14-12 (SEQ ID NO:483), DOM14-13 (SEQ ID NO:48), DOM14-14 (SEQ ID NO:485), DOM14-15 (SEQ ID NO:486), DOM14-16 (SEQ ID NO:487), DOM14-17 (SEQ ID NO:488), DOM14-18 (SEQ ID NO:489), DOM14-19 (SEQ ID NO:490), DOM14-2 (SEQ ID NO:478), DOM14-20 (SEQ ID NO:491), DOM14-21 (SEQ ID NO:492), DOM14-22 (SEQ ID NO:493), DOM14-23 (SEQ ID NO:494), DOM14-24 (SEQ ID NO:495), DOM14-25 (SEQ ID NO:496), DOM14-26 (SEQ ID NO:497), DOM14-27 (SEQ ID NO:498), DOM14-28 (SEQ ID NO:499), DOM14-3 (SEQ ID NO:479), DOM14-31 (SEQ ID NO:500), DOM14-32 (SEQ ID NO:501), DOM14-33 (SEQ ID NO:502), DOM14-34 (SEQ ID NO:503), DOM14-35 (SEQ ID NO:504), DOM14-36 (SEQ ID NO:505), DOM14-37 (SEQ ID NO:506), DOM14-38 (SEQ ID NO:507), DOM14-39 (SEQ ID NO:508), DOM14-4 (SEQ ID NO:480), DOM14-40 (SEQ ID NO:509), DOM14-41 (SEQ ID NO:510), DOM14-42 (SEQ ID NO:511), DOM14-43 (SEQ ID NO:512), DOM14-44 (SEQ ID NO:513), DOM14-45 (SEQ ID NO:514), DOM14-46 (SEQ ID NO:515), DOM14-47 (SEQ ID NO:516), DOM14-48 (SEQ ID NO:517), DOM14-49 (SEQ ID NO:518), DOM14-50 (SEQ ID NO:519), DOM14-51 (SEQ ID NO:520), DOM14-52 (SEQ ID NO:521), DOM14-53 (SEQ ID NO:522), DOM14-54 (SEQ ID NO:523), DOM14-55 (SEQ ID NO:524), DOM14-56 (SEQ ID NO:525), DOM14-57 (SEQ ID NO:526), DOM14-58 (SEQ ID NO:527), DOM14-59 (SEQ ID NO:528), DOM14-60 (SEQ ID NO:529), DOM14-61 (SEQ ID NO:530), DOM14-62 (SEQ ID NO:531), DOM14-63 (SEQ ID NO:532), DOM14-64 (SEQ ID NO:533), DOM14-65 (SEQ ID NO:534), DOM14-66 (SEQ ID NO:535), DOM14-67 (SEQ ID NO:536), DOM14-70 (SEQ ID NO:539), DOM14-68 (SEQ ID NO:537), and DOM14-69 (SEQ ID NO:538).

In some embodiments, the polypeptide domain that has a binding site with binding specificity for CD56 comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence or a dAb selected from the group consisting of: DOM14-1 (SEQ ID NO:477), DOM14-10 (SEQ ID NO:481), DOM14-100 (SEQ ID NO:540), DOM14-11 (SEQ ID NO:482), DOM14-12 (SEQ ID NO:483), DOM14-13 (SEQ ID NO:484), DOM14-14 (SEQ ID NO:485), DOM14-15 (SEQ ID NO:486), DOM14-16 (SEQ ID NO:487), DOM14-17 (SEQ ID NO:488), DOM14-18 (SEQ ID NO:489), DOM14-19 (SEQ ID NO:490), DOM14-2 (SEQ ID NO:478), DOM14-20 (SEQ ID NO:491), DOM14-21 (SEQ ID NO:492), DOM14-22 (SEQ ID NO:493), DOM14-23 (SEQ ID NO:494), DOM14-24 (SEQ ID NO:495), DOM14-25 (SEQ ID NO:496), DOM14-26 (SEQ ID NO:497), DOM14-27 (SEQ ID NO:498), DOM14-28 (SEQ ID NO:499), DOM14-3 (SEQ ID NO:479), DOM14-31 (SEQ ID NO:500), DOM14-32 (SEQ ID NO:501), DOM14-33 (SEQ ID NO:502), DOM14-34 (SEQ ID NO:503), DOM14-35 (SEQ ID NO:504), DOM14-36 (SEQ ID NO:505), DOM14-37 (SEQ ID NO:506), DOM14-38 (SEQ ID NO:507), DOM14-39 (SEQ ID NO:508), DOM14-4 (SEQ ID NO:480), DOM14-40 (SEQ ID NO:509), DOM14-41 (SEQ ID NO:510), DOM14-42 (SEQ ID NO:511), DOM14-43 (SEQ ID NO:512), DOM14-44 (SEQ ID NO:513), DOM14-45 (SEQ ID NO:514), DOM14-46 (SEQ ID NO:515), DOM14-47 (SEQ ID NO:516), DOM14-48 (SEQ ID NO:517), DOM14-49 (SEQ ID NO:518), DOM14-50 (SEQ ID NO:519), DOM14-51 (SEQ ID NO:520), DOM14-52 (SEQ ID NO:521), DOM14-53 (SEQ ID NO:522), DOM14-54 (SEQ ID NO:523), DOM14-55 (SEQ ID NO:524), DOM14-56 (SEQ ID NO:525), DOM14-57 (SEQ ID NO:526), DOM14-58 (SEQ ID NO:527), DOM14-59 (SEQ ID NO:528), DOM14-60 (SEQ ID NO:529), DOM14-61 (SEQ ID NO:530), DOM14-62 (SEQ ID NO:531), DOM14-63 (SEQ ID NO:532), DOM14-64 (SEQ ID NO:533), DOM14-65 (SEQ ID NO:534), DOM14-66 (SEQ ID NO:535), DOM14-67 (SEQ ID NO:536), DOM14-70 (SEQ ID NO:539), DOM14-68 (SEQ ID NO:537), and DOM14-69 (SEQ ID NO:538).

In preferred embodiments, the polypeptide domain that has a binding site with binding specificity for CD56 is selected from the group consisting of: DOM14-23 (SEQ ID NO: 494), DOM14-48 (SEQ ID NO:517), DOM14-56 (SEQ ID NO:525), DOM14-57 (SEQ ID NO:526), DOM14-62 (SEQ ID NO:531), DOM14-63 (SEQ ID NO:532), DOM14-68 (SEQ ID NO:537), and DOM14-70 (SEQ ID NO: 539). In some embodiments, the polypeptide domain that has a binding site with binding specificity for CD56 competes with any of the dAbs disclosed herein for binding to CD56.

The polypeptide domain that has a binding site with binding specificity for CD56 can comprise any suitable immunoglobulin variable domain, and preferably comprises a human variable domain or a variable domain that comprises human framework regions. In certain embodiments, the polypeptide domain that has a binding site with binding specificity for CD56 comprises a universal framework, as described herein.

In certain embodiments, the polypeptide domain that has a binding site with binding specificity for CD56 resists aggregation, unfolds reversibly and/or comprises a framework region and is secreted as described above for the polypeptide domain that has a binding site with binding specificity for CD38.

Ligands with dAb Monomers that Bind Serum Albumin

The ligands of the invention can further comprise a dAb monomer that binds serum albumin (SA) with a K_{d} of 1 nM to 500 μM (i.e., 1×10⁻⁹ to 5×10⁻⁴), preferably 100 nM to 10 μM. Preferably, for a ligand comprising anti-SA dAb, the binding (e.g., K_{d} and/or K_{off} as measured by surface plasmon resonance, (e.g., using BiaCore)) of the ligand to its target(s) is from 1 to 100000 times (preferably 100 to 100000, more preferably 1000 to 100000, or 10000 to 100000 times) stronger than for SA. Preferably, the serum albumin is human serum albumin (HSA). In one embodiment, the first dAb (or a dAb monomer) binds SA (e.g., HSA) with a K_{d} of approximately 50, preferably 70, and more preferably 100, 150 or 200 nM.

In certain embodiments, the dAb monomer that binds SA resists aggregation, unfolds reversibly and/or comprises a framework region, as described above for dAb monomers that bind CD38.

In particular embodiments, the antigen-binding fragment of an antibody that binds serum albumin is a dAb that binds human serum albumin. In certain embodiments, the dAb binds human serum albumin and competes for binding to albumin with a dAb selected from the group consisting of: DOM7m-16 (SEQ ID NO: 541), DOM7m-12 (SEQ ID NO: 542), DOM7m-26 (SEQ ID NO: 543), DOM7r-1 (SEQ ID NO: 544), DOM7r-3 (SEQ ID NO: 545), DOM7r-4 (SEQ ID NO: 546), DOM7r-5 (SEQ ID NO: 547), DOM7r-7 (SEQ ID NO: 548), DOM7r-8 (SEQ ID NO: 549), DOM7h-2 (SEQ ID NO: 550), DOM7h-3 (SEQ ID NO: 551), DOM7h-4 (SEQ ID NO: 552), DOM7h-6 (SEQ ID NO: 553), DOM7h-1 (SEQ ID NO: 555), DOM7h-7 (SEQ ID NO: 477), DOM7h-8 (SEQ ID NO: 564), DOM7r-13 (SEQ ID NO: 565), DOM7r-14 (SEQ ID NO: 566), DOM7h-22 (SEQ ID NO: 557), DOM7h-23 (SEQ ID NO: 558), DOM7h-24 (SEQ ID NO: 559), DOM7h-25 (SEQ ID NO: 560), DOM7h-26 (SEQ ID NO: 561), DOM7h-21 (SEQ ID NO: 562), DOM7h-27 (SEQ ID NO: 563), DOM7r-15 (SEQ ID NO: 567), DOM7r-16 (SEQ ID NO: 568), DOM7r-17 (SEQ ID NO: 569), DOM7r-18 (SEQ ID NO: 570), DOM7r-19 (SEQ ID NO: 571), DOM7r-20 (SEQ ID NO: 572), DOM7r-21 (SEQ ID NO: 573), DOM7r-22 (SEQ ID NO: 574), DOM7r-23 (SEQ ID NO: 575), DOM7r-24 (SEQ ID NO: 576), DOM7r-25 (SEQ ID NO: 577), DOM7r-26 (SEQ ID NO: 578), DOM7r-27 (SEQ ID NO: 579), DOM7r-28 (SEQ ID NO: 580), DOM7r-29 (SEQ ID NO: 581), DOM7r-30 (SEQ ID NO: 582), DOM7r-31 (SEQ ID NO: 583), DOM7r-32 (SEQ ID NO: 584), and DOM7r-33 (SEQ ID NO: 585).

In certain embodiments, the dAb binds human serum albumin and comprises an amino acid sequence that has at least about 80%, or at least about 85%, or at least about 90%, or at least about 95%, or at least about 96%, or at least about 97%, or at least about 98%, or at least about 99% amino acid sequence identity with the amino acid sequence of a dAb selected from the group consisting of DOM7m-16 (SEQ ID NO: 541), DOM7m-12 (SEQ ID NO: 542), DOM7m-26 (SEQ ID NO: 543), DOM7r-1 (SEQ ID NO: 544), DOM7r-3 (SEQ ID NO: 545), DOM7r-4 (SEQ ID NO: 546), DOM7r-5 (SEQ ID NO: 547), DOM7r-7 (SEQ ID NO: 548), DOM7r-8 (SEQ ID NO: 549), DOM7h-2 (SEQ ID NO: 550), DOM7h-3 (SEQ ID NO: 551), DOM7h-4 (SEQ ID NO: 552), DOM7h-6 (SEQ ID NO: 553), DOM7h-1 (SEQ ID NO: 555), DOM7h-7 (SEQ ID NO: 477), DOM7h-8 (SEQ ID NO: 564), DOM7r-13 (SEQ ID NO: 565), DOM7r-14 (SEQ ID NO: 566), DOM7h-22 (SEQ ID NO: 557), DOM7h-23 (SEQ ID NO: 558), DOM7h-24 (SEQ ID NO: 559), DOM7h-25 (SEQ ID NO: 560), DOM7h-26 (SEQ ID NO: 561), DOM7h-21 (SEQ ID NO: 562), DOM7h-27 (SEQ ID NO: 563), DOM7r-15 (SEQ ID NO: 567), DOM7r-16 (SEQ ID NO: 568), DOM7r-17 (SEQ ID NO: 569), DOM7r-18 (SEQ ID NO: 570), DOM7r-19 (SEQ ID NO: 571), DOM7r-20 (SEQ ID NO: 572), DOM7r-21 (SEQ ID NO: 573), DOM7r-22 (SEQ ID NO: 574), DOM7r-23 (SEQ ID NO: 575), DOM7r-24 (SEQ ID NO: 576), DOM7r-25 (SEQ ID NO: 577), DOM7r-26 (SEQ ID NO: 578), DOM7r-27 (SEQ ID NO: 579), DOM7r-28 (SEQ ID NO: 580), DOM7r-29 (SEQ ID NO: 581), DOM7r-30 (SEQ ID NO: 582), DOM7r-31 (SEQ ID NO: 583), DOM7r-32 (SEQ ID NO: 584), and DOM7r-33 (SEQ ID NO: 585).

For example, the dAb that binds human serum albumin can comprise an amino acid sequence that has at least about 90%, or at least about 95%, or at least about 96%, or at least about 97%, or at least about 98%, or at least about 99% amino acid sequence identity with DOM7h-2 (SEQ ID NO: 550), DOM71h-3 (SEQ ID NO: 551), DOM7h-4 (SEQ ID NO: 552), DOM7h-6 (SEQ ID NO: 553), DOM7h-1 (SEQ ID NO: 554), DOM7h-7 (SEQ ID NO: 555), DOM7h-8 (SEQ ID NO: 564), DOM7r-13 (SEQ ID NO: 565), DOM7r-14 (SEQ ID NO: 566), DOM7h-22 (SEQ ID NO: 557), DOM7h-23 (SEQ ID NO: 558), DOM7h-24 (SEQ ID NO: 559), DOM7h-25 (SEQ ID NO: 560), DOM7h-26 (SEQ ID NO: 561), DOM7b-21 (SEQ ID NO: 562), and DOM7h-27 (SEQ ID NO: 563)

Amino acid sequence identity is preferably determined using a suitable sequence alignment algorithm and default parameters, such as BLAST P (Karlin and Altschul, Proc. Natl. Acad. Sci. USA 87(6):2264-2268 (1990)).

In more particular embodiments, the dAb is a VK dAb that binds human serum albumin and has a amino acid sequence selected from the group consisting of DOM7h-2 (SEQ ID NO: 550), DOM7h-3 (SEQ ID NO: 551), DOM7h-4 (SEQ ID NO: 552), DOM7h-6 (SEQ ID NO: 553), DOM7h-1 (SEQ ID NO: 554), DOM7h-7 (SEQ ID NO: 555), DOM7h-8 (SEQ ID NO: 564), DOM7r-13 (SEQ ID NO: 565), and DOM7r-14 (SEQ ID NO: 566), or a V_{H} dAb that has an amino acid sequence selected from the group consisting of: DOM7h-22 (SEQ ID NO: 557), DOM7h-23 (SEQ ID NO: 558), DOM7h-24 (SEQ ID NO: 559), DOM7h-25 (SEQ ID NO: 560), DOM7h-26 (SEQ ID NO: 561), DOM7h-21 (SEQ ID NO: 562), DOM7h-27 (SEQ ID NO: 563). In other embodiments, the antigen-binding fragment of an antibody that binds serum albumin is a dAb that binds human serum albumin and comprises the CDRs of any of the foregoing amino acid sequences.

Suitable Camelid V_{HH} that bind serum albumin include those disclosed in WO 2004/041862 (Ablynx N.V.) and herein Sequence A (SEQ ID NO: 586), Sequence B (SEQ ID NO: 587), Sequence C (SEQ ID NO: 588), Sequence D (SEQ ID NO: 589), Sequence E (SEQ ID NO: 590), Sequence F (SEQ ID NO: 591), Sequence G (SEQ ID NO: 592), Sequence H (SEQ ID NO: 593), Sequence I (SEQ ID NO: 594), Sequence J (SEQ ID NO: 595), Sequence K (SEQ ID NO: 596), Sequence L (SEQ ID NO: 597), Sequence M (SEQ ID NO: 598), Sequence N (SEQ ID NO: 599), Sequence 0 (SEQ ID NO: 600), Sequence P (SEQ ID NO: 601), Sequence Q (SEQ ID NO: 602). In certain embodiments, the Camelid V_{HH} binds human serum albumin and comprises an amino acid sequence that has at least about 80%, or at least about 85%, or at least about 90%, or at least about 95%, or at least about 96%, or at least about 97%, or at least about 98%, or at least about 99% amino acid sequence identity with any one of SEQ ID NOS: 586-602. Amino acid sequence identity is preferably determined using a suitable sequence alignment algorithm and default parameters, such as BLAST P (Karlin and Altschul, Proc. Natl. Acad. Sci. USA 87(6):2264-2268 (1990)).

In some embodiments, the ligand comprises an anti-serum albumin dAb that competes with any anti-serum albumin dAb disclosed herein for binding to serum albumin (e.g., human serum albumin).

### Nucleic Acid Molecules, Vectors and Host Cells

The invention also provides isolated and/or recombinant nucleic acid molecules encoding ligands (dual-specific ligands and multispecific ligands), as described herein.

In certain embodiments, the isolated and/or recombinant nucleic acid comprises a nucleotide sequence encoding a ligand as described herein comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, or at least about 99% homologous to the amino acid sequence selected from the group consisting of: DOM11-14 (SEQ ID NO: 242), DOM11-22 (SEQ ID NO:246), DOM11-23 (SEQ ID NO:247), DOM11-25 (SEQ ID NO:249), DOM11-26 (SEQ ID NO:250), DOM11-27 (SEQ ID NO:251), DOM11-29 (SEQ ID NO:253), DOM11-3 (SEQ ID NO:234), DOM11-30 (SEQ ID NO:254), DOM11-31 (SEQ ID NO:255), DOM11-32 (SEQ ID NO:256), DOM11-36 (SEQ ID NO:260), DOM11-4 (SEQ ID NO:235), DOM11-43 (SEQ ID NO:266), DOM11-44 (SEQ ID NO:267), DOM11-45 (SEQ ID NO:268), DOM11-5 (SEQ ID NO:236), DOM11-7 (SEQ ID NO:238), DOM11-1 (SEQ ID NO:232), DOM11-10 (SEQ ID NO:241), DOM11-16 (SEQ ID NO:243), DOM11-2 (SEQ ID NO:233), DOM11-20 (SEQ ID NO:244), DOM11-21 (SEQ ID NO:245), DOM11-24 (SEQ ID NO:248), DOM11-28 (SEQ ID NO:252), DOM11-33 (SEQ ID NO:257), DOM11-34 (SEQ ID NO:258), DOM11-35 (SEQ ID NO:259), DOM11-37 (SEQ ID NO:261), DOM11-38 (SEQ ID NO:262), DOM11-39 (SEQ ID NO:293), DOM11-41 (SEQ ID NO:264), DOM11-42 (SEQ ID NO:265), DOM11-6 (SEQ ID NO:237), DOM11-8 (SEQ ID NO:239), DOM11-9 (SEQ ID NO:240), DOM12-1 (SEQ ID NO:306), DOM12-15 (SEQ ID NO:317), DOM12-17 (SEQ ID NO:318), DOM12-19 (SEQ ID NO:320), DOM12-2 (SEQ ID NO:307), DOM12-20 (SEQ ID NO:321), DOM12-21 (SEQ ID NO:322), DOM12-22 (SEQ ID NO:323), DOM12-3 (SEQ ID NO:308), DOM12-33 (SEQ ID NO:334), DOM12-39 (SEQ ID NO:340), DOM12-4 (SEQ ID NO:309), DOM12-40 (SEQ ID NO:341), DOM12-41 (SEQ ID NO:342), DOM12-42 (SEQ ID NO:343), DOM12-44 (SEQ ID NO:345), DOM12-46 (SEQ ID NO:347), DOM12-6 (SEQ ID NO:311), DOM12-7 (SEQ ID NO:312), DOM12-10 (SEQ ID NO:315), DOM12-11 (SEQ ID NO:316), DOM12-18 (SEQ ID NO:319), DOM12-23 (SEQ ID NO:324), DOM12-24 (SEQ ID NO:325), DOM12-25 (SEQ ID NO:326), DOM12-26 (SEQ ID NO:327), DOM12-27 (SEQ ID NO:328), DOM12-28 (SEQ ID NO:329), DOM12-29, (SEQ ID NO:330), DOM12-30 (SEQ ID NO:331), DOM12-31 (SEQ ID NO:332), DOM12-32 (SEQ ID NO:333), DOM12-34 (SEQ ID NO:335), DOM12-35 (SEQ ID NO:336), DOM12-36 (SEQ ID NO:337), DOM12-37 (SEQ ID NO:338), DOM12-38 (SEQ ID NO:339), DOM12-43 (SEQ ID NO:344), DOM12-45 (SEQ ID NO:346), DOM12-5 (SEQ ID NO:310), DOM12-8 (SEQ ID NO:313), DOM12-9 (SEQ ID NO:314), DOM13-1 (SEQ ID NO:385), DOM13-12 (SEQ ID NO:393), DOM13-13 (SEQ ID NO:394), DOM13-14 (SEQ ID NO:395), DOM13-15 (SEQ ID NO:3396), DOM13-16 (SEQ ID NO:397), DOM13-17 (SEQ ID NO:398), DOM13-18 (SEQ ID NO:399), DOM13-19 (SEQ ID NO:400), DOM13-2 (SEQ ID NO:386), DOM13-20 (SEQ ID NO:401), DOM13-21 (SEQ ID NO:402), DOM13-22 (SEQ ID NO:403), DOM13-23 (SEQ ID NO:404), DOM13-24 (SEQ ID NO:3405), DOM13-25 (SEQ ID NO:406), DOM13-26 (SEQ ID NO:407), DOM13-27 (SEQ ID NO:408), DOM13-28 (SEQ ID NO:409), DOM13-29 (SEQ ID NO:410), DOM13-3 (SEQ ID NO:387), DOM13-30 (SEQ ID NO:411), DOM13-31 (SEQ ID NO:412), DOM13-32 (SEQ ID NO:413), DOM13-33 (SEQ ID NO:414), DOM-13-34 (SEQ ID NO:415), DOM13-35 (SEQ ID NO:416), DOM13-36 (SEQ ID NO:417), DOM13-37 (SEQ ID NO:418), DOM13-4 (SEQ ID NO:388), DOM13-42 (SEQ ID NO:419), DOM13-43 (SEQ ID NO:420), DOM13-44 (SEQ ID NO:421), DOM13-45 (SEQ ID NO:422), DOM13-46 (SEQ ID NO:423), DOM13-47 (SEQ ID NO:424), DOM13-48 (SEQ ID NO:425), DOM13-49 (SEQ ID NO:426), DOM13-5 (SEQ ID NO:389), DOM13-50 (SEQ ID NO:427), DOM13-51 (SEQ ID NO:428), DOM13-52 (SEQ ID NO:429), DOM13-53 (SEQ ID NO:430), DOM13-54 (SEQ ID NO:431), DOM13-55 (SEQ ID NO:432), DOM13-56 (SEQ ID NO:433), DOM13-57 (SEQ ID NO:434), DOM13-58 (SEQ ID NO:435), DOM13-59 (SEQ ID NO:436), DOM13-6 (SEQ ID NO:390), DOM13-60 (SEQ ID NO:437), DOM13-61 (SEQ ID NO:438), DOM13-62 (SEQ ID NO:439), DOM13-63 (SEQ ID NO:440), DOM13-64 (SEQ ID NO:441), DOM13-65 (SEQ ID NO:442), DOM13-66 (SEQ ID NO:443), DOM13-67 (SEQ ID NO: 444), DOM13-68 (SEQ ID NO: 445), DOM13-69 (SEQ ID NO: 446), DOM13-7 (SEQ ID NO: 391), DOM13-70 (SEQ ID NO: 447), DOM13-71 (SEQ ID NO: 448), DOM13-72 (SEQ ID NO:449), DOM13-73 (SEQ ID NO:450), DOM13-74 (SEQ ID NO:451), DOM13-75 (SEQ ID NO:452), DOM13-76 (SEQ ID NO:453), DOM13-77 (SEQ ID NO:454), DOM13-78 (SEQ ID NO:455), DOM13-79 (SEQ ID NO:456), DOM13-8 (SEQ ID NO:392), DOM13-80 (SEQ ID NO:457), DOM13-81 (SEQ ID NO:458), DOM13-82 (SEQ ID NO:459), DOM13-83 (SEQ ID NO:460), DOM13-84 (SEQ ID NO:461), DOM13-85 (SEQ ID NO:462), DOM13-86 (SEQ ID NO:463), DOM13-87 (SEQ ID NO:464), DOM13-88 (SEQ ID NO:465), DOM13-89 (SEQ ID NO:466), DOM13-90 (SEQ ID NO:467), DOM13-91 (SEQ ID NO:468), DOM13-92 (SEQ ID NO:469), DOM13-93 (SEQ ID NO:470), DOM13-94 (SEQ ID NO:471), DOM13-95 (SEQ ID NO:472), DOM14-1 (SEQ ID NO:477), DOM14-10 (SEQ ID NO:481), DOM14-100 (SEQ ID NO:540), DOM14-11 (SEQ ID NO:482), DOM14-12 (SEQ ID NO:483), DOM14-13 (SEQ ID NO:484), DOM14-14 (SEQ ID NO:485), DOM14-15 (SEQ ID NO:486), DOM14-16 (SEQ ID NO:487), DOM14-17 (SEQ ID NO:488), DOM14-18 (SEQ ID NO:489), DOM14-19 (SEQ ID NO:490), DOM14-2 (SEQ ID NO:478), DOM14-20 (SEQ ID NO:491), DOM14-21 (SEQ ID NO:492), DOM14-22 (SEQ ID NO:493), DOM14-23 (SEQ ID NO:494), DOM14-24 (SEQ ID NO:495), DOM14-25 (SEQ ID NO:496), DOM14-26 (SEQ ID NO:497), DOM14-27 (SEQ ID NO:498), DOM14-28 (SEQ ID NO:499), DOM14-3 (SEQ ID NO:479), DOM14-31 (SEQ ID NO:500), DOM14-32 (SEQ ID NO:501), DOM14-33 (SEQ ID NO:502), DOM14-34 (SEQ ID NO:503), DOM14-35 (SEQ ID NO:504), DOM14-36 (SEQ ID NO:505), DOM14-37 (SEQ ID NO:506), DOM14-38 (SEQ ID NO:507), DOM14-39 (SEQ ID NO:508), DOM14-4 (SEQ ID NO:480), DOM14-40 (SEQ ID NO:509), DOM14-41 (SEQ ID NO:510), DOM14-42 (SEQ ID NO:511), DOM14-43 (SEQ ID NO:512), DOM14-44 (SEQ ID NO:513), DOM14-45 (SEQ ID NO:514), DOM14-46 (SEQ ID NO:515), DOM14-47 (SEQ ID NO:516), DOM14-48 (SEQ ID NO:517), DOM14-49 (SEQ ID NO:518), DOM14-50 (SEQ ID NO:519), DOM14-51 (SEQ ID NO:520), DOM14-52 (SEQ ID NO:521), DOM14-53 (SEQ ID NO:522), DOM14-54 (SEQ ID NO:523), DOM14-55 (SEQ ID NO:524), DOM14-56 (SEQ ID NO:525), DOM14-57 (SEQ ID NO:526), DOM14-58 (SEQ ID NO:527), DOM14-59 (SEQ ID NO:528), DOM14-60 (SEQ ID NO:529), DOM14-61 (SEQ ID NO:530), DOM14-62 (SEQ ID NO:531), DOM14-63 (SEQ ID NO:532), DOM14-64 (SEQ ID NO:533), DOM14-65 (SEQ ID NO:534), DOM14-66 (SEQ ID NO:535), DOM14-67 (SEQ ID NO:536), DOM14-70 (SEQ ID NO:539), DOM14-68 (SEQ ID NO:537), and DOM14-69 (SEQ ID NO:538).

In certain embodiments, the isolated and/or recombinant nucleic acid comprises a nucleotide sequence that encodes a ligand, as described herein, wherein said nucleotide sequence has at least about 80%, at least about 85%, at least about 90%, at least about 91%, at least about 92%, at least about 93%, at least about 94%, at least about 95%, at least about 96%, at least about 97%, at least about 98%, or at least about 99% nucleotide sequence identity with a nucleotide sequence selected from the group consisting of: DOM11-14 (SEQ ID NO: 10), DOM11-22 (SEQ ID NO: 11), DOM11-23 (SEQ ID NO: 3), DOM11-25 (SEQ ID NO: 12), DOM11-26 (SEQ ID NO: 13), DOM11-27 (SEQ ID NO: 14), DOM11-29 (SEQ ID NO: 15), DOM11-3 (SEQ ID NO: 1), DOM11-30 (SEQ ID NO: 2), DOM11-31 (SEQ ID NO: 16), DOM11-32 (SEQ ID NO: 7), DOM11-36 (SEQ ID NO: 17), DOM11-4 (SEQ ID NO: 18), DOM11-43 (SEQ ID NO: 19), DOM11-44 (SEQ ID NO:20), DOM11-45 (SEQ ID NO: 21), DOM11-5 (SEQ ID NO: 22), DOM11-7 (SEQ ID NO: 4), DOM11-1 (SEQ ID NO: 23), DOM11-10 (SEQ ID NO: 24), DOM11-16 (SEQ ID NO:25), DOM11-2 (SEQ ID NO: 26), DOM11-20 (SEQ ID NO: 27), DOM11-21 (SEQ ID NO:28), DOM11-24 (SEQ ID NO:9), DOM11-28 (SEQ ID NO:29), DOM11-33 (SEQ ID NO: 30), DOM11-34 (SEQ ID NO: 31), DOM11-35 (SEQ ID NO:32), DOM11-37 (SEQ ID NO: 8), DOM11-38 (SEQ ID NO: 5), DOM11-39 (SEQ ID NO: 6), DOM11-41 (SEQ ID NO: 33), DOM11-42 (SEQ ID NO: 34), DOM11-6 (SEQ ID NO: 35), DOM11-8 (SEQ ID NO:36), DOM11-9 (SEQ ID NO: 37), DOM12-1 (SEQ ID NO: 41), DOM12-15 (SEQ ID NO: 42), DOM12-17 (SEQ ID NO: 39), DOM12-19 (SEQ ID NO: 43), DOM12-2 (SEQ ID NO: 44), DOM12-20 (SEQ ID NO: 45), DOM12-21 (SEQ ID NO: 46), DOM12-22 (SEQ ID NO: 47), DOM12-3 (SEQ ID NO: 48), DOM12-33 (SEQ ID NO:49), DOM12-39 (SEQ ID NO: 50), DOM12-4 (SEQ ID NO: 51), DOM12-40 (SEQ ID NO: 52), DOM12-41 (SEQ ID NO: 53), DOM12-42 (SEQ ID NO:54), DOM12-44 (SEQ ID NO: 55), DOM12-46 (SEQ ID NO: 56), DOM12-6 (SEQ ID NO: 57), DOM12-7 (SEQ ID NO: 58), DOM12-10 (SEQ ID NO:59), DOM12-11 (SEQ ID NO:60), DOM12-18 (SEQ ID NO: 61), DOM12-23 (SEQ ID NO: 62), DOM12-24 (SEQ ID NO: 63), DOM12-25 (SEQ ID NO: 64), DOM12-26 (SEQ ID NO:40), DOM12-27 (SEQ ID NO: 65), DOM12-28 (SEQ ID NO:66), DOM12-29 (SEQ ID NO: 67), DOM12-30 (SEQ ID NO: 68), DOM12-31 (SEQ ID NO:69), DOM12-32 (SEQ ID NO: 70), DOM12-34 (SEQ ID NO:71), DOM12-35 (SEQ ID NO: 72), DOM12-36 (SEQ ID NO:73), DOM12-37 (SEQ ID NO:74), DOM12-38 (SEQ ID NO:75), DOM12-43 (SEQ ID NO:76), DOM12-45 (SEQ ID NO: 38), DOM12-5 (SEQ ID NO: 77), DOM12-8 (SEQ ID NO: 78), DOM12-9 (SEQ ID NO: 79), DOM13-1 (SEQ ID NO: 89), DOM13-12 (SEQ ID NO:90), DOM13-13 (SEQ ID NO: 91), DOM13-14 (SEQ ID NO: 92), DOM13-15 (SEQ ID NO:93), DOM13-16 (SEQ ID NO:94), DOM13-17 (SEQ ID NO: 95), DOM13-18 (SEQ ID NO:96), DOM13-19 (SEQ ID NO:97), DOM13-2 (SEQ ID NO: 98), DOM13-20 (SEQ ID NO:99), DOM13-21 (SEQ ID NO: 100), DOM13-22 (SEQ ID NO:101), DOM13-23 (SEQ ID NO: 102), DOM13-24 (SEQ ID NO: 103), DOM13-25 (SEQ ID NO:80), DOM13-26 (SEQ ID NO: 104), DOM13-27 (SEQ ID NO:105), DOM13-28 (SEQ ID NO:106), DOM13-29 (SEQ ID NO:104), DOM13-3 (SEQ ID NO: 108), DOM13-30 (SEQ ID NO: 109), DOM13-31 (SEQ ID NO: 110), DOM13-32 (SEQ ID NO: 111), DOM13-33 (SEQ ID NO: 112), DOM-13-34 (SEQ ID NO: 113), DOM13-35 (SEQ ID NO: 114), DOM13-36 (SEQ ID NO: 115), DOM13-37 (SEQ ID NO:116), DOM13-4 (SEQ ID NO:117), DOM13-42 (SEQ ID NO: 118), DOM13-43 (SEQ ID NO:119), DOM13-44 (SEQ ID NO:120), DOM13-45 (SEQ ID NO: 121), DOM13-46 (SEQ ID NO:122), DOM13-47 (SEQ ID NO: 123), DOM13-48 (SEQ ID NO: 124), DOM13-49 (SEQ ID NO:125), DOM13-5 (SEQ ID NO: 126), DOM13-50 (SEQ ID NO: 127), DOM13-51 (SEQ ID NO: 128), DOM13-52 (SEQ ID NO:129), DOM13-53 (SEQ ID NO:130), DOM13-54 (SEQ ID NO:131), DOM13-55 (SEQ ID NO:132), DOM13-56 (SEQ ID NO:133), DOM13-57 (SEQ ID NO: 81), DOM13-58 (SEQ ID NO: 82), DOM13-59 (SEQ ID NO: 83), DOM13-6 (SEQ ID NO:134), DOM13-60 (SEQ ID NO:135), DOM13-61 (SEQ ID NO: 136), DOM13-62 (SEQ ID NO:137), DOM13-63 (SEQ ID NO: 138), DOM13-64 (SEQ ID NO: 84), DOM13-65 (SEQ ID NO: 85), DOM13-66 (SEQ ID NO:139), DOM13-67 (SEQ ID NO: 140), DOM13-68 (SEQ ID NO: 141), DOM13-69 (SEQ ID NO:142), DOM13-7 (SEQ ID NO: 143), DOM13-70 (SEQ ID NO: 144), DOM13-71 (SEQ ID NO: 145), DOM13-72 (SEQ ID NO:146), DOM13-73 (SEQ ID NO:147), DOM13-74 (SEQ ID NO: 86), DOM13-75 (SEQ ID NO:148), DOM13-76 (SEQ ID NO: 149), DOM13-77 (SEQ ID NO:150), DOM13-78 (SEQ ID NO: 151), DOM13-79 (SEQ ID NO: 152), DOM13-8 (SEQ ID NO:153), DOM13-80 (SEQ ID NO:154), DOM13-81 (SEQ ID NO: 155), DOM13-82 (SEQ ID NO: 156), DOM13-83 (SEQ ID NO:157), DOM13-84 (SEQ ID NO:158), DOM13-85 (SEQ ID NO:159), DOM13-86 (SEQ ID NO: 160), DOM13-87 (SEQ ID NO: 161), DOM13-88 (SEQ ID NO: 162), DOM13-89 (SEQ ID NO: 163), DOM13-90 (SEQ ID NO:164), DOM13-91 (SEQ ID NO:165), DOM13-92 (SEQ ID NO: 166), DOM13-93 (SEQ ID NO: 87), DOM13-94 (SEQ ID NO: 167), DOM13-95 (SEQ ID NO:88), DOM14-1 (SEQ ID NO: 176), DOM14-10 (SEQ ID NO: 177), DOM14-100 (SEQ ID NO:178), DOM14-11 (SEQ ID NO: 179), DOM14-12 (SEQ ID NO: 180), DOM14-13 (SEQ ID NO: 181), DOM14-14 (SEQ ID NO: 182), DOM14-15 (SEQ ID NO: 183), DOM14-16 (SEQ ID NO:184), DOM14-17 (SEQ ID NO: 185), DOM14-18 (SEQ ID NO: 186), DOM14-19 (SEQ ID NO:187), DOM14-2 (SEQ ID NO: 188), DOM14-20 (SEQ ID NO:189), DOM14-21 (SEQ ID NO: 190), DOM14-22 (SEQ ID NO:191), DOM14-23 (SEQ ID NO: 168), DOM14-24 (SEQ ID NO: 192), DOM14-25 (SEQ ID NO:193), DOM14-26 (SEQ ID NO: 194), DOM14-27 (SEQ ID NO: 195), DOM14-28 (SEQ ID NO:196), DOM14-3 (SEQ ID NO:197), DOM14-31 (SEQ ID NO:198), DOM14-32 (SEQ ID NO: 199), DOM14-33 (SEQ ID NO: 200), DOM14-34 (SEQ ID NO: 201), DOM14-35 (SEQ ID NO:202), DOM14-36 (SEQ ID NO: 203), DOM14-37 (SEQ ID NO:204), DOM14-38 (SEQ ID NO: 205), DOM14-39 (SEQ ID NO: 206), DOM14-4 (SEQ ID NO: 207), DOM14-40 (SEQ ID NO: 208), DOM14-41 (SEQ ID NO: 209), DOM14-42 (SEQ ID NO:210), DOM14-43 (SEQ ID NO: 211), DOM14-44 (SEQ ID NO:212), DOM14-45 (SEQ ID NO:213), DOM14-46 (SEQ ID NO: 214), DOM14-47 (SEQ ID NO:215), DOM14-48 (SEQ ID NO: 169), DOM14-49 (SEQ ID NO: 216), DOM14-50 (SEQ ID NO: 217), DOM14-51 (SEQ ID NO:218), DOM14-52 (SEQ ID NO:219), DOM14-53 (SEQ ID NO:220), DOM14-54 (SEQ ID NO:221), DOM14-55 (SEQ ID NO: 222), DOM14-56 (SEQ ID NO: 170), DOM14-57 (SEQ ID NO: 171), DOM14-58 (SEQ ID NO:223), DOM14-59 (SEQ ID NO:224), DOM14-60 (SEQ ID NO:225), DOM14-61 (SEQ ID NO: 226), DOM14-62 (SEQ ID NO: 172), DOM14-63 (SEQ ID NO: 173), DOM14-64 (SEQ ID NO: 227), DOM14-65 (SEQ ID NO:228), DOM14-66 (SEQ ID NO: 229), DOM14-67 (SEQ ID NO:230), DOM14-70 (SEQ ID NO:175), DOM14-68 (SEQ ID NO:174), and DOM14-69 (SEQ ID NO:231).

The invention also provides a vector comprising a recombinant nucleic acid molecule of the invention. In certain embodiments, the vector is an expression vector comprising one or more expression control elements or sequences that are operably linked to the recombinant nucleic acid of the invention. The invention also provides a recombinant host cell comprising a recombinant nucleic acid molecule or vector of the invention. Suitable vectors (e.g., plasmids, phagmids), expression control elements, host cells and methods for producing recombinant host cells of the invention are well-known in the art, and examples are further described herein.

Suitable expression vectors can contain a number of components, for example, an origin of replication, a selectable marker gene, one or more expression control elements, such as a transcription control element (e.g., promoter, enhancer, terminator) and/or one or more translation signals, a signal sequence or leader sequence, and the like. Expression control elements and a signal sequence, if present, can be provided by the vector or other source. For example, the transcriptional and/or translational control sequences of a cloned nucleic acid encoding an antibody chain can be used to direct expression.

A promoter can be provided for expression in a desired host cell. Promoters can be constitutive or inducible. For example, a promoter can be operably linked to a nucleic acid encoding an antibody, antibody chain or portion thereof, such that it directs transcription of the nucleic acid. A variety of suitable promoters for procaryotic (e.g., lac, tac, T3, T7 promoters for E. coli) and eucaryotic (e.g., simian virus 40 early or late promoter, Rous sarcoma virus long terminal repeat promoter, cytomegalovirus promoter, adenovirus late promoter) hosts are available.

In addition, expression vectors typically comprise a selectable marker for selection of host cells carrying the vector, and, in the case of a replicable expression vector, an origin or replication. Genes encoding products which confer antibiotic or drug resistance are common selectable markers and may be used in procaryotic (e.g., lactamase gene (ampicillin resistance), Tet gene for tetracycline resistance) and eucaryotic cells (e.g., neomycin (G418 or geneticin), gpt (mycophenolic acid), ampicillin, or hygromycin resistance genes). Dihydrofolate reductase marker genes permit selection with methotrexate in a variety of hosts. Genes encoding the gene product of auxotrophic markers of the host (e.g., LEU2, URA3, HIS3) are often used as selectable markers in yeast. Use of viral (e.g., baculovirus) or phage vectors, and vectors which are capable of integrating into the genome of the host cell, such as retroviral vectors, are also contemplated. Suitable expression vectors for expression in mammalian cells and prokaryotic cells (E. coli), insect cells (Drosophila Schnieder S2 cells, Sf9) and yeast (P. methanolica, P. pastoris, S. cerevisiae) are well-known in the art.

Suitable host cells can be prokaryotic, including bacterial cells such as E. coli, B. subtilis and/or other suitable bacteria; eukaryotic cells, such as fungal or yeast cells (e.g., Pichia pastoris, Aspergillus sp., Saccharomyces cerevisiae, Schizosaccharomyces po be, Neurospora crassa), or other lower eukaryotic cells, and cells of higher eukaryotes such as those from insects (e.g., Drosophila Schnieder S2 cells, Sf9 insect cells (WO 94/26087 (O'Connor)), mammals (e.g., COS cells, such as COS-1 (ATCC Accession No. CRL-1650) and COS-7 (ATCC Accession No. CRL-1651), CHO (e.g., ATCC Accession No. CRL-9096, CHO DG44 (Urlaub, G. and Chasin, L A., Proc. Natl. Acac. Sci. USA, 77(7):4216-4220 (1980))), 293 (ATCC Accession No. CRL-1573), HeLa (ATCC Accession No. CCL-2), CV1 (ATCC Accession No. CCL-70), WOP (Dailey, L., et al., J. Virol., 54:739-749 (1985), 3T3, 293T (Pear, W. S., et al., Proc. Natl. Acad. Sci. U.S.A., 90:8392-8396 (1993)) NSO cells, SP2/0, HuT 78 cells and the like, or plants (e.g., tobacco). (See, for example, Ausubel, F. M. et al., eds. Current Protocols in Molecular Biology, Greene Publishing Associates and John Wiley & Sons Inc. (1993).) In some embodiments, the host cell is an isolated host cell and is not part of a multicellular organism (e.g., plant or animal). In preferred embodiments, the host cell is a non-human host cell.

The invention also provides a method for producing a ligand (e.g., dual-specific ligand, multispecific ligand) of the invention, comprising maintaining a recombinant host cell comprising a recombinant nucleic acid of the invention under conditions suitable for expression of the recombinant nucleic acid, whereby the recombinant nucleic acid is expressed and a ligand is produced. In some embodiments, the method further comprises isolating the ligand.

### Preparation of Immunoglobulin Based Ligands

Ligands (e.g., dual specific ligands, multi specific) according to the invention can be prepared according to previously established techniques, used in the field of antibody engineering, for the preparation of scFv, “phage” antibodies and other engineered antibody molecules. Techniques for the preparation of antibodies are for example described in the following reviews and the references cited therein: Winter & Milstein, (1991) Nature 349:293-299; Pluckthun (1992) Immunological Reviews 13 0:151-188; Wright et al., (1992) Crti. Rev. Immunol. 12:125-168; Holliger, P. & Winter, G. (1993) Curr. Op. Biotechn. 4, 446-449; Carter, et al. (1995) J. Hematother. 4, 463-470; Chester, K. A. & Hawkins, R. E. (1995) Trends Biotechn. 13, 294-300; Hoogenboom, H. R. (1997) Nature Biotechnol. 15, 125-126; Fearon, D. (1997) Nature Biotechnol. 15, 618-619; Pluckthun, A. & Pack, P. (1997) Immunotechnology 3, 83-105; Carter, P. & Merchant, A. M. (1997) Cirr. Opin. Biotechnol. 8, 449-454; Holliger, P. & Winter, G. (1997) Cancer Immunol. Immunother. 45, 128-130.

Suitable techniques employed for selection of antibody variable domains with a desired specificity employ libraries and selection procedures which are known in the art. Natural libraries (Marks et al. (1991) J. Mol. Biol., 222: 581; Vaughan et al. (1996) Nature Biotech., 14: 309) which use rearranged V genes harvested from human B cells are well known to those skilled in the art. Synthetic libraries (Hoogenboom & Winter (1992) J. Mol. Biol., 227: 381; Barbas et al. (1992) Proc. Natl. Acad. Sci. USA, 89: 4457; Nissim et al. (1994) EMBO J., 13: 692; Griffiths et al. (1994) EMBO J., 13: 3245; De Kruif et al. (1995) J. Mol. Biol., 248: 97) are prepared by cloning immunoglobulin V genes, usually using PCR. Errors in the PCR process can lead to a high degree of randomisation. V_{H} and/or V_{L} libraries may be selected against target antigens or epitopes separately, in which case single domain binding is directly selected for, or together.

### Library Vector Systems

A variety of selection systems are known in the art which are suitable for use in the present invention. Examples of such systems are described below.

Bacteriophage lambda expression systems may be screened directly as bacteriophage plaques or as colonies of lysogens, both as previously described (Huse et al. (1989) Science, 246: 1275; Caton and Koprowski (1990) Proc. Natl. Acad. Sci. U.S.A., 87; Mullinax et al. (1990) Proc. Natl. Acad. Sci. U.S.A., 87: 8095; Persson et al. (1991) Proc. Natl. Acad. Sci. U.S.A., 88: 2432) and are of use in the invention. Whilst such expression systems can be used to screen up to 106 different members of a library, they are not really suited to screening of larger numbers (greater than 106 members). Of particular use in the construction of libraries are selection display systems, which enable a nucleic acid to be linked to the polypeptide it expresses. As used herein, a selection display system is a system that permits the selection, by suitable display means, of the individual members of the library by binding the generic and/or target.

Selection protocols for isolating desired members of large libraries are known in the art, as typified by phage display techniques. Such systems, in which diverse peptide sequences are displayed on the surface of filamentous bacteriophage (Scott and Smith (1990) Science, 249: 386), have proven useful for creating libraries of antibody fragments (and the nucleotide sequences that encode them) for the in vitro selection and amplification of specific antibody fragments that bind a target antigen (McCafferty et al., WO 92/01047). The nucleotide sequences encoding the variable regions are linked to gene fragments which encode leader signals that direct them to the periplasmic space of E. coli and as a result the resultant antibody fragments are displayed on the surface of the bacteriophage, typically as fusions to bacteriophage coat proteins (e.g., pIII or pVIII). Alternatively, antibody fragments are displayed externally on lambda phage capsids (phagebodies). An advantage of phage-based display systems is that, because they are biological systems, selected library members can be amplified simply by growing the phage containing the selected library member in bacterial cells. Furthermore, since the nucleotide sequence that encode the polypeptide library member is contained on a phage or phagemid vector, sequencing, expression and subsequent genetic manipulation is relatively straightforward.

Methods for the construction of bacteriophage antibody display libraries and lambda phage expression libraries are well known in the art (McCafferty et al. (1990) Nature, 348: 552; Kang et al. (1991) Proc. Natl. Acad. Sci. U.S.A., 88: 4363; Clackson et al. (1991) Nature, 352: 624; Lowman et al. (1991) Biochemistry, 30: 10832; Burton et al. (1991) Proc. Natl. Acad. Sci. U.S.A., 88: 10134; Hoogenboom et al. (1991) Nucleic Acids Res., 19: 4133; Chang et al. (1991) J. Immunol., 147: 3610; Breitling et al. (1991) Gene, 104: 147; Marks et al. (1991) supra; Barbas et al. (1992) supra; Hawkins and Winter (1992) J. Immunol., 22: 867; Marks et al., 1992, J. Biol. Chem., 267: 16007; Lerner et al. (1992) Science, 258: 1313, incorporated herein by reference).

One particularly advantageous approach has been the use of scFv phage-libraries (Huston et al., 1988, Proc. Natl. Acad. Sci. U.S.A., 85: 5879-5883; Chaudhary et al. (1990) Proc. Natl. Acad. Sci. U.S.A., 87:1066-1070; McCafferty et al. (1990) supra; Clackson et al. (1991) Nature, 352: 624; Marks et al. (1991) J. Mol. Biol., 222: 581; Chiswell et al. (1992) Trends Biotech., 10: 80; Marks et al. (1992) J. Biol. Chenz., 267). Various embodiments of scFv libraries displayed on bacteriophage coat proteins have been described. Refinements of phage display approaches are also known, for example as described in WO96/06213 and WO92/01047 (Medical Research Council et al.) and WO97/08320 (Morphosys), which are incorporated herein by reference.

Other systems for generating libraries of polypeptides involve the use of cell-free enzymatic machinery for the in vitro synthesis of the library members. In one method, RNA molecules are selected by alternate rounds of selection against a target and PCR amplification (Tuerk and Gold (1990) Science, 249: 505; Ellington and Szostak (1990) Nature, 346: 818). A similar technique may be used to identify DNA sequences which bind a predetermined human transcription factor (Thiesen and Bach (1990) Nucleic Acids Res., 18: 3203; Beaudry and Joyce (1992) Science, 257: 635; WO92/05258 and WO92/14843). In a similar way, in vitro translation can be used to synthesise polypeptides as a method for generating large libraries. These methods which generally comprise stabilised polysome complexes, are described further in WO88/08453, WO90/05785, WO90/07003, WO91/02076, WO91/05058, and WO92/02536. Alternative display systems which are not phage-based, such as those disclosed in WO95/22625 and WO95/11922 (Affymax) use the polysomes to display polypeptides for selection.

A still further category of techniques involves the selection of repertoires in artificial compartments, which allow the linkage of a gene with its gene product. For example, a selection system in which nucleic acids encoding desirable gene products may be selected in microcapsules formed by water-in-oil emulsions is described in WO99/02671, WO00/40712 and Tawfik & Griffiths (1998) Nature Biotechnol 16(7), 652-6. Genetic elements encoding a gene product having a desired activity are compartmentalised into microcapsules and then transcribed and/or translated to produce their respective gene products (RNA or protein) within the microcapsules. Genetic elements which produce gene product having desired activity are subsequently sorted. This approach selects gene products of interest by detecting the desired activity by a variety of means.

### Library Construction

Libraries intended for selection, may be constructed using techniques known in the art, for example as set forth above, or may be purchased from commercial sources. Libraries which are useful in the present invention are described, for example, in WO99/20749. Once a vector system is chosen and one or more nucleic acid sequences encoding polypeptides of interest are cloned into the library vector, one may generate diversity within the cloned molecules by undertaking mutagenesis prior to expression; alternatively, the encoded proteins may be expressed and selected, as described above, before mutagenesis and additional rounds of selection are performed. Mutagenesis of nucleic acid sequences encoding structurally optimized polypeptides is carried out by standard molecular methods. Of particular use is the polymerase chain reaction, or PCR, (Mullis and Faloona (1987) Methods Enzymol., 155: 335, herein incorporated by reference). PCR, which uses multiple cycles of DNA replication catalyzed by a thermostable, DNA-dependent DNA polymerase to amplify the target sequence of interest, is well known in the art. The construction of various antibody libraries has been discussed in Winter et al. (1994) Ann. Rev. Immunology 12, 433-55, and references cited therein.

PCR is performed using template DNA (at least 1 fg; more usefully, 1-1000 ng) and at least 25 pmol of oligonucleotide primers; it may be advantageous to use a larger amount of primer when the primer pool is heavily heterogeneous, as each sequence is represented by only a small fraction of the molecules of the pool, and amounts become limiting in the later amplification cycles. A typical reaction mixture includes: 2 μl of DNA, 25 μmol of oligonucleotide primer, 2.5 μl of 10×PCR buffer 1 (Perkin-Elmer, Foster City, Calif.), 0.4 μl of 1.25 μM dNTP, 0.15 μl (or 2.5 units) of Taq DNA polymerase (Perkin Elmer, Foster City, Calif.) and deionized water to a total volume of 25 μl. Mineral oil is overlaid and the PCR is performed using a programmable thermal cycler. The length and temperature of each step of a PCR cycle, as well as the number of cycles, is adjusted in accordance to the stringency requirements in effect. Annealing temperature and timing are determined both by the efficiency with which a primer is expected to anneal to a template and the degree of mismatch that is to be tolerated; obviously, when nucleic acid molecules are simultaneously amplified and mutagenised, mismatch is required, at least in the first round of synthesis. The ability to optimise the stringency of primer annealing conditions is well within the knowledge of one of moderate skill in the art. An annealing temperature of between 30° C. and 72° C. is used. Initial denaturation of the template molecules normally occurs at between 92° C. and 99° C. for 4 minutes, followed by 20-40 cycles consisting of denaturation (94-99° C. for 15 seconds to 1 minute), annealing (temperature determined as discussed above; 1-2 minutes), and extension (72° C. for 1-5 minutes, depending on the length of the amplified product). Final extension is generally for 4 minutes at 72° C., and may be followed by an indefinite (0-24 hour) step at 4° C.

### Combining Single Variable Domains

Domains useful in the invention, once selected, may be combined by a variety of methods known in the art, including covalent and non-covalent methods. Preferred methods include the use of polypeptide linkers, as described, for example, in connection with scFv molecules (Bird et al., (1988) Science 242:423-426). Discussion of suitable linkers is provided in Bird et al. Science 242, 423-426; Hudson et al, Journal Immunol Methods 231 (1999) 177-189; Hudson et al, Proc Nat Acad Sci USA 85, 5879-5883. Linkers are preferably flexible, allowing the two single domains to interact. One linker example is a (Gly₄ Ser)ₙ linker, where n−1 to 8, e.g., 2, 3, 4, 5 or 7. The linkers used in diabodies, which are less flexible, may also be employed (Holliger et al., (1993) Proc. Nat. Acad. Sci. USA 90:6444-6448). In one embodiment, the linker employed is not an immunoglobulin hinge region.

Variable domains may be combined using methods other than linkers. For example, the use of disulphide bridges, provided through naturally-occurring or engineered cysteine residues, may be exploited to stabilize V_{H}-V_{H}, V_{L}-V_{L} or V_{H}-V_{L} dimers (Reiter et al., (1994) Protein Eng. 7: 697-704) or by remodelling the interface between the variable domains to improve the “fit” and thus the stability of interaction (Ridgeway et al., (1996) Protein Eng. 7: 617-621; Zhu et al., (1997) Protein Science 6:781-788). Other techniques for joining or stabilizing variable domains of immunoglobulins, and in particular antibody V_{H} domains, may be employed as appropriate.

### Characterisation of Ligands

The binding of a dual-specific ligand to the cell or the binding of each binding domain to each specific target can be tested by methods which will be familiar to those skilled in the art and include ELISA. In a preferred embodiment of the invention binding is tested using monoclonal phage ELISA. Phage ELISA may be performed according to any suitable procedure: an exemplary protocol is set forth below.

Populations of phage produced at each round of selection can be screened for binding by ELISA to the selected antigen or epitope, to identify “polyclonal” phage antibodies. Phage from single infected bacterial colonies from these populations can then be screened by ELISA to identify “monoclonal” phage antibodies. It is also desirable to screen soluble antibody fragments for binding to antigen or epitope, and this can also be undertaken by ELISA using reagents, for example, against a C- or N-terminal tag (see for example Winter et al. (1994) Ann. Rev. immunology 12, 433-55 and references cited therein.

The diversity of the selected phage monoclonal antibodies may also be assessed by gel electrophoresis of PCR products (Marks et al. 1991, supra; Nissim et al. 1994 supra), probing (Tomlinson et al., 1992) J. Mol. Biol. 227, 776) or by sequencing of the vector DNA.

### Structure of Ligands

In the case that each variable domains is selected from V-gene repertoires, for instance, using phage display technology as herein described, then these variable domains comprise a universal framework region, such that is they may be recognized by a specific generic dual-specific ligand as herein defined. The use of universal frameworks, generic ligands and the like is described in WO99/20749.

Where V-gene repertoires are used variation in polypeptide sequence is preferably located within the structural loops of the variable domains. The polypeptide sequences of either variable domain may be altered by DNA shuffling or by mutation in order to enhance the interaction of each variable domain with its complementary pair. DNA shuffling is known in the art and taught, for example, by Stemmer, 1994, Nature 370: 389-391 and U.S. Pat. No. 6,297,053, both of which are incorporated herein by reference. Other methods of mutagenesis are well known to those of skill in the art.

In general, nucleic acid molecules and vector constructs required for selection, preparation and formatting dual-specific ligands may be constructed and manipulated as set forth in standard laboratory manuals, such as Sambrook et al. (1989) Molecular Cloning: A Laboratory Manual, Cold Spring Harbor, USA.

The manipulation of nucleic acids useful in the present invention is typically carried out in recombinant vectors. As used herein, vector refers to a discrete element that is used to introduce heterologous DNA into cells for the expression and/or replication thereof. Methods by which to select or construct and, subsequently, use such vectors are well known to one of ordinary skill in the art. Numerous vectors are publicly available, including bacterial plasmids, bacteriophage, artificial chromosomes and episomal vectors. Such vectors may be used for simple cloning and mutagenesis; alternatively gene expression vector is employed. A vector of use according to the invention may be selected to accommodate a polypeptide coding sequence of a desired size, typically from 0.25 kilobase (kb) to 40 kb or more in length A suitable host cell is transformed with the vector after in vitro cloning manipulations. Each vector contains various functional components, which generally include a cloning (or “polylinker”) site, an origin of replication and at least one selectable marker gene. If given vector is an expression vector, it additionally possesses one or more of the following: enhancer element, promoter, transcription termination and signal sequences, each positioned in the vicinity of the cloning site, such that they are operatively linked to the gene encoding a dual-specific ligand according to the invention.

Both cloning and expression vectors generally contain nucleic acid sequences that enable the vector to replicate in one or more selected host cells. Typically in cloning vectors, this sequence is one that enables the vector to replicate independently of the host chromosomal DNA and includes origins of replication or autonomously replicating sequences. Such sequences are well known for a variety of bacteria, yeast and viruses. The origin of replication from the plasmid pBR322 is suitable for most Gram-negative bacteria, the 2 micron plasmid origin is suitable for yeast, and various viral origins (e.g., SV 40, adenovirus) are useful for cloning vectors in mammalian cells. Generally, the origin of replication is not needed for mammalian expression vectors unless these are used in mammalian cells able to replicate high levels of DNA, such as COS cells.

Advantageously, a cloning or expression vector may contain a selection gene also referred to as selectable marker. This gene encodes a protein necessary for the survival or growth of transformed host cells grown in a selective culture medium. Host cells not transformed with the vector containing the selection gene will therefore not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available in the growth media.

Since the replication of vectors encoding a dual-specific ligand according to the present invention is most conveniently performed in E. coli, an E. coli-selectable marker, for example, the β-lactamase gene that confers resistance to the antibiotic ampicillin, is of use. These can be obtained from E. coli plasmids, such as pBR322 or a pUC plasmid such as pUC18 or pUC19.

Expression vectors usually contain a promoter that is recognised by the host organism and is operably linked to the coding sequence of interest. Such a promoter may be inducible or constitutive. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Promoters suitable for use with prokaryotic hosts include, for example, the β-lactamase and lactose promoter systems, alkaline phosphatase, the tryptophan (trp) promoter system and hybrid promoters such as the tac promoter. Promoters for use in bacterial systems will also generally contain a Shine-Delgarno sequence operably linked to the coding sequence.

The preferred vectors are expression vectors that enable the expression of a nucleotide sequence corresponding to a polypeptide library member. Thus, selection with the first and/or second antigen or epitope can be performed by separate propagation and expression of a single clone expressing the polypeptide library member or by use of any selection display system. As described above, the preferred selection display system is bacteriophage display. Thus, phage or phagemid vectors may be used, e.g., pIT1 or pIT2. Leader sequences useful in the invention include pelB, stII, ompA, phoA, bla and pelA. One example are phagemid vectors which have an E. coli. origin of replication (for double stranded replication) and also a phage origin of replication (for production of single-stranded DNA). The manipulation and expression of such vectors is well known in the art (Hoogenboom and Winter (1992) supra; Nissim et al. (1994) supra). Briefly, the vector contains a β-lactamase gene to confer selectivity on the phagemid and a lac promoter upstream of a expression cassette that consists (N to C terminal) of a pelB leader sequence (which directs the expressed polypeptide to the periplasmic space), a multiple cloning site (for cloning the nucleotide version of the library member), optionally, one or more peptide tag (for detection), optionally, one or more TAG stop codon and the phage protein pIII. Thus, using various suppressor and non-suppressor strains of E. coli and with the addition of glucose, iso-propyl thio-β-D-galactoside (IPTG) or a helper phage, such as VCS M13, the vector is able to replicate as a plasmid with no expression, produce large quantities of the polypeptide library member only or produce phage, some of which contain at least one copy of the polypeptide-pIII fusion on their surface.

Construction of vectors encoding dual-specific ligands according to the invention employs conventional ligation techniques. Isolated vectors or DNA fragments are cleaved, tailored, and religated in the form desired to generate the required vector. If desired, analysis to confirm that the correct sequences are present in the constructed vector can be performed in a known fashion. Suitable methods for constructing expression vectors, preparing in vitro transcripts, introducing DNA into host cells, and performing analyses for assessing expression and function are known to those skilled in the art. The presence of a gene sequence in a sample is detected, or its amplification and/or expression quantified by conventional methods, such as Southern or Northern analysis, Western blotting, dot blotting of DNA, RNA or protein, in situ hybridisation, immunocytochemistry or sequence analysis of nucleic acid or protein molecules. Those skilled in the art will readily envisage how these methods may be modified, if desired.

### Skeletons

Skeletons may be based on immunoglobulin molecules or may be non-immunoglobulin in origin as set forth above. Each domain of the dual-specific ligand may be a different skeleton. Preferred immunoglobulin skeletons as herein defined includes any one or more of those selected from the following: an immunoglobulin molecule comprising at least (i) the CL (kappa or lambda subclass) domain of an antibody; or (ii) the CH1 domain of an antibody heavy chain; an immunoglobulin molecule comprising the CH1 and CH2 domains of an antibody heavy chain; an immunoglobulin molecule comprising the CH1, CH2 and CH3 domains of an antibody heavy chain; or any of the subset (ii) in conjunction with the CL (kappa or lambda subclass) domain of an antibody. A hinge region domain may also be included. Such combinations of domains may, for example, mimic natural antibodies, such as IgG or IgM, or fragments thereof, such as Fv, scFv, Fab or F(ab′)₂ molecules. Those skilled in the art will be aware that this list is not intended to be exhaustive.

### Protein Scaffolds

Each binding domain comprises a protein scaffold and one or more CDRs which are involved in the specific interaction of the domain with one or more epitopes. Advantageously, an epitope binding domain according to the present invention comprises three CDRs. Suitable protein scaffolds include any of those selected from the group consisting of the following: those based on immunoglobulin domains, those based on fibronectin, those based on affibodies, those based on CTLA4, those based on chaperones such as GroEL, those based on lipocallin and those based on the bacterial Fc receptors SpA and SpD. Those skilled in the art will appreciate that this list is not intended to be exhaustive.

### Scaffolds for use in Constructing Ligands

Selection of the Main-Chain Conformation

The members of the immunoglobulin superfamily all share a similar fold for their polypeptide chain. For example, although antibodies are highly diverse in terms of their primary sequence, comparison of sequences and crystallographic structures has revealed that, contrary to expectation, five of the six antigen binding loops of antibodies (H1, H2, L1, L2, L3) adopt a limited number of main-chain conformations, or canonical structures (Chothia and Lesk (1987) J. Mol. Biol., 196: 901; Chothia et al. (1989) Nature, 342: 877). Analysis of loop lengths and key residues has therefore enabled prediction of the main-chain conformations of H1, H2, L1, L2 and L3 found in the majority of human antibodies (Chothia et al. (1992) J. Mol. Biol., 227: 799; Tomlinson et al. (1995) EMBO J., 14: 4628; Williams et al. (1996) J. Mol. Biol., 264: 220). Although the H3 region is much more diverse in terms of sequence, length and structure (due to the use of D segments), it also forms a limited number of main-chain conformations for short loop lengths which depend on the length and the presence of particular residues, or types of residue, at key positions in the loop and the antibody framework (Martin et al. (1996) J. Mol. Biol., 263: 800; Shirai et al. (1996) FEBS Letters, 399: 1).

Libraries of ligands and/or binding domains can be designed in which certain loop lengths and key residues have been chosen to ensure that the main-chain conformation of the members is known. Advantageously, these are real conformations of immunoglobulin superfamily molecules found in nature, to minimize the chances that they are non-functional, as discussed above. Germline V gene segments serve as one suitable basic framework for constructing antibody or T-cell receptor libraries; other sequences are also of use. Variations may occur at a low frequency, such that a small number of functional members may possess an altered main-chain conformation, which does not affect its function.

Canonical structure theory is also of use to assess the number of different main-chain conformations encoded by ligands, to predict the main-chain conformation based on dual-specific ligand sequences and to choose residues for diversification which do not affect the canonical structure. It is known that, in the human V_{κ} domain, the L1 loop can adopt one of four canonical structures, the L2 loop has a single canonical structure and that 90% of human V_{κ} domains adopt one of four or five canonical structures for the L3 loop (Tomlinson et al. (1995) supra); thus, in the V_{κ} domain alone, different canonical structures can combine to create a range of different main-chain conformations. Given that the V_{λ} domain encodes a different range of canonical structures for the L1, L2 and L3 loops and that VK and V_{λ} domains can pair with any V_{H} domain which can encode several canonical structures for the H1 and H2 loops, the number of canonical structure combinations observed for these five loops is very large. This implies that the generation of diversity in the main-chain conformation may be essential for the production of a wide range of binding specificities. However, by constructing an antibody library based on a single known main-chain conformation it has been found, contrary to expectation, that diversity in the main-chain conformation is not required to generate sufficient diversity to target substantially all antigens. Even more surprisingly, the single main-chain conformation need not be a consensus structure—a single naturally occurring conformation can be used as the basis for an entire library. Thus, in a preferred aspect, the ligands of the invention possess a single known main-chain conformation.

The single main-chain conformation that is chosen is preferably commonplace among molecules of the immunoglobulin superfamily type in question. A conformation is commonplace when a significant number of naturally occurring molecules are observed to adopt it. Accordingly, in a preferred aspect of the invention, the natural occurrence of the different main-chain conformations for each binding loop of an immunoglobulin domain are considered separately and then a naturally occurring variable domain is chosen which possesses the desired combination of main-chain conformations for the different loops. If none is available, the nearest equivalent may be chosen. It is preferable that the desired combination of main-chain conformations for the different loops is created by selecting germline gene segments which encode the desired main-chain conformations. It is more preferable, that the selected germline gene segments are frequently expressed in nature, and most preferable that they are the most frequently expressed of all natural germline gene segments.

In designing ligands (e.g., ds-dAbs) or libraries thereof the incidence of the different main-chain conformations for each of the six antigen binding loops may be considered separately. For H1, H2, L1, L2 and L3, a given conformation that is adopted by between 20% and 100% of the antigen binding loops of naturally occurring molecules is chosen. Typically, its observed incidence is above 35% (i.e. between 35% and 100%) and, ideally, above 50% or even above 65%. Since the vast majority of H3 loops do not have canonical structures, it is preferable to select a main-chain conformation which is commonplace among those loops which do display canonical structures. For each of the loops, the conformation which is observed most often in the natural repertoire is therefore selected. In human antibodies, the most popular canonical structures (CS) for each loop are as follows: H1—CS1 (79% of the expressed repertoire), H2—CS 3 (46%), L1—CS 2 of V_{κ} (39%), L2—CS1 (100%), L3—CS1 of V_{κ} (36%) (calculation assumes a κ:λ ratio of 70:30, Hood et al. (1967) Cold Spring Harbor Symp. Quant. Biol., 48: 133). For H3 loops that have canonical structures, a CDR3 length (Kabat et al. (1991) Sequences of proteins of immunological interest, U.S. Department of Health and Human Services) of seven residues with a salt-bridge from residue 94 to residue 101 appears to be the most common. There are at least 16 human antibody sequences in the EMBL data library with the required H3 length and key residues to form this conformation and at least two crystallographic structures in the protein data bank which can be used as a basis for antibody modelling (2 cgr and 1 tet). The most frequently expressed germline gene segments that this combination of canonical structures are the V_{H} segment 3-23 (DP-47), the J_{H} segment JH4b, the V_{κ} segment O2/O12 (DPK9) and the J_{κ} segment J_{κ}1. V_{H} segments DP45 and DP38 are also suitable. These segments can therefore be used in combination as a basis to construct a library with the desired single main-chain conformation.

Alternatively, instead of choosing the single main-chain conformation based on the natural occurrence of the different main-chain conformations for each of the binding loops in isolation, the natural occurrence of combinations of main-chain conformations is used as the basis for choosing the single main-chain conformation. In the case of antibodies, for example, the natural occurrence of canonical structure combinations for any two, three, four, five or for all six of the antigen binding loops can be determined. Here, it is preferable that the chosen conformation is commonplace in naturally occurring antibodies and most preferable that it is observed most frequently in the natural repertoire. Thus, in human antibodies, for example, when natural combinations of the five antigen binding loops, H1, H2, L1, L2 and L3, are considered, the most frequent combination of canonical structures is determined and then combined with the most popular conformation for the H3 loop, as a basis for choosing the single main-chain conformation.

### Diversification of the Canonical Sequence

Having selected several known main-chain conformations or, preferably a single known main-chain conformation, dual-specific ligands (e.g., ds-dAbs) or libraries for use in the invention can be constructed by varying each binding site of the molecule in order to generate a repertoire with structural and/or functional diversity. This means that variants are generated such that they possess sufficient diversity in their structure and/or in their function so that they are capable of providing a range of activities.

The desired diversity is typically generated by varying the selected molecule at one or more positions. The positions to be changed can be chosen at random or are preferably selected. The variation can then be achieved either by randomisation, during which the resident amino acid is replaced by any amino acid or analogue thereof, natural or synthetic, producing a very large number of variants or by replacing the resident amino acid with one or more of a defined subset of amino acids, producing a more limited number of variants.

Various methods have been reported for introducing such diversity. Error-prone PCR (Hawkins et al. (1992) J. Mol. Biol., 226: 889), chemical mutagenesis (Deng et al. (1994) J. Biol. Chem., 269: 9533) or bacterial mutator strains (Low et al. (1996) J. Mol. Biol., 260: 359) can be used to introduce random mutations into the genes that encode the molecule. Methods for mutating selected positions are also well known in the art and include the use of mismatched oligonucleotides or degenerate oligonucleotides, with or without the use of PCR. For example, several synthetic antibody libraries have been created by targeting mutations to the antigen binding loops. The H3 region of a human tetanus toxoid-binding Fab has been randomised to create a range of new binding specificities (Barbas et al. (1992) Proc. Natl. Acad. Sci. USA, 89: 4457). Random or semi-random H3 and L3 regions have been appended to germline V gene segments to produce large libraries with unmutated framework regions (Hoogenboom & Winter (1992) J. Mol. Biol., 227: 381; Barbas et al. (1992) Proc. Natl. Acad. Sci. USA, 89: 4457; Nissim et al. (1994) EMBO J., 13: 692; Griffiths et al. (1994) EMBO J., 13: 3245; De Kruif et al. (1995) J. Mol. Biol., 248: 97). Such diversification has been extended to include some or all of the other antigen binding loops (Crameri et al. (1996) Nature Med., 2: 100; Riechmann et al. (1995) Bio/Technology, 13: 475; Morphosys, WO97/08320, supra).

Since loop randomization has the potential to create approximately more than 10¹⁵ structures for H3 alone and a similarly large number of variants for the other five loops, it is not feasible using current transformation technology or even by using cell free systems to produce a library representing all possible combinations. For example, in one of the largest libraries constructed to date, 6×10¹⁰ different antibodies, which is only a fraction of the potential diversity for a library of this design, were generated (Griffiths et al. (1994) supra).

Preferably, only the residues that are directly involved in creating or modifying the desired function of each domain of the dual-specific ligand molecule are diversified. For many molecules, the function of each domain will be to bind a target and therefore diversity should be concentrated in the target binding site, while avoiding changing residues which are crucial to the overall packing of the molecule or to maintaining the chosen main-chain conformation.

### Diversification of the Canonical Sequence as it Applies to Antibody Domains

In the case of antibody based ligands (e.g., ds-dAbs), the binding site for each target is most often the antigen binding site. Thus, preferably only those residues in the antigen binding site are varied. These residues are extremely diverse in the human antibody repertoire and are known to make contacts in high-resolution antibody/antigen complexes. For example, in L2 it is known that positions 50 and 53 are diverse in naturally occurring antibodies and are observed to make contact with the antigen. In contrast, the conventional approach would have been to diversify all the residues in the corresponding Complementarity Determining Region (CDR1) as defined by Kabat et al. (1991, supra), some seven residues compared to the two diversified in the library for use according to the invention. This represents a significant improvement in terms of the functional diversity required to create a range of antigen binding specificities.

In nature, antibody diversity is the result of two processes: somatic recombination of germline V, D and J gene segments to create a naive primary repertoire (so called germline and junctional diversity) and somatic hypermutation of the resulting rearranged V genes. Analysis of human antibody sequences has shown that diversity in the primary repertoire is focused at the centre of the antigen binding site whereas somatic hypermutation spreads diversity to regions at the periphery of the antigen binding site that are highly conserved in the primary repertoire (see Tomlinson et al. (1996) J. Mol. Biol., 256: 813). This complementarity has probably evolved as an efficient strategy for searching sequence space and, although apparently unique to antibodies, it can easily be applied to other polypeptide repertoires. The residues which are varied are a subset of those that form the binding site for the target. Different (including overlapping) subsets of residues in the target binding site are diversified at different stages during selection, if desired.

In the case of an antibody repertoire, an initial ‘naive’ repertoire can be created where some, but not all, of the residues in the antigen binding site are diversified. As used herein in this context, the term “naive” refers to antibody molecules that have no pre-determined target. These molecules resemble those which are encoded by the immunoglobulin genes of an individual who has not undergone immune diversification, as is the case with fetal and newborn individuals, whose immune systems have not yet been challenged by a wide variety of antigenic stimuli. This repertoire is then selected against a range of antigens or epitopes. If required, further diversity can then be introduced outside the region diversified in the initial repertoire. This matured repertoire can be selected for modified function, specificity or affinity.

Naive repertoires of binding domains for the construction of dual-specific ligands in which some or all of the residues in the antigen binding site are varied are known in the art. (See, WO 2004/058821, WO 2004/003019, and WO 03/002609). The “primary” library mimics the natural primary repertoire, with diversity restricted to residues at the centre of the antigen binding site that are diverse in the germline V gene segments (germline diversity) or diversified during the recombination process (junctional diversity). Those residues which are diversified include, but are not limited to, H50, H52, H52a, H53, H55, H56, H58, H95, H96, H97, H98, L50, L53, L91, L92, L93, L94 and L96. In the “somatic” library, diversity is restricted to residues that are diversified during the recombination process (junctional diversity) or are highly somatically mutated. Those residues which are diversified include, but are not limited to: H31, H33, H35, H95, H96, H97, H98, L30, L31, L32, L34 and L96. All the residues listed above as suitable for diversification in these libraries are known to make contacts in one or more antibody-antigen complexes. Since in both libraries, not all of the residues in the antigen binding site are varied, additional diversity is incorporated during selection by varying the remaining residues, if it is desired to do so. It shall be apparent to one skilled in the art that any subset of any of these residues (or additional residues which comprise the antigen binding site) can be used for the initial and/or subsequent diversification of the antigen binding site.

In the construction of libraries for use in the invention, diversification of chosen positions is typically achieved at the nucleic acid level, by altering the coding sequence which specifies the sequence of the polypeptide such that a number of possible amino acids (all 20 or a subset thereof) can be incorporated at that position. Using the IUPAC nomenclature, the most versatile codon is NNK, which encodes all amino acids as well as the TAG stop codon. The NNK codon is preferably used in order to introduce the required diversity. Other codons which achieve the same ends are also of use, including the NNN codon, which leads to the production of the additional stop codons TGA and TAA.

A feature of side-chain diversity in the antigen binding site of human antibodies is a pronounced bias which favors certain amino acid residues. If the amino acid composition of the ten most diverse positions in each of the V_{H}, V_{κ}, and V_{λ} regions are summed, more than 76% of the side-chain diversity comes from only seven different residues, these being, serine (24%), tyrosine (14%), asparagine (11%), glycine (9%), alanine (7%), aspartate (6%) and threonine (6%). This bias towards hydrophilic residues and small residues which can provide main-chain flexibility probably reflects the evolution of surfaces which are predisposed to binding a wide range of antigens or epitopes and may help to explain the required promiscuity of antibodies in the primary repertoire.

Since it is preferable to mimic this distribution of amino acids, the distribution of amino acids at the positions to be varied preferably mimics that seen in the antigen binding site of antibodies. Such bias in the substitution of amino acids that permits selection of certain polypeptides (not just antibody polypeptides) against a range of target antigens is easily applied to any polypeptide repertoire. There are various methods for biasing the amino acid distribution at the position to be varied (including the use of tri-nucleotide mutagenesis, see WO97/08320), of which the preferred method, due to ease of synthesis, is the use of conventional degenerate codons. By comparing the amino acid profile encoded by all combinations of degenerate codons (with single, double, triple and quadruple degeneracy in equal ratios at each position) with the natural amino acid use it is possible to calculate the most representative codon. The codons (AGT)(AGC)T, (AGT)(AGC)C and (AGT)(AGC)(CT)—that is, DVT, DVC and DVY, respectively using IUPAC nomenclature—are those closest to the desired amino acid profile: they encode 22% serine and 11% tyrosine, asparagine, glycine, alanine, aspartate, threonine and cysteine. Preferably, therefore, libraries are constructed using either the DVT, DVC or DVY codon at each of the diversified positions.

### Therapeutic and Diagnostic Compositions and Uses

The invention provides compositions comprising the ligands of the invention and a pharmaceutically acceptable carrier, diluent or excipient, and therapeutic and diagnostic methods that employ the ligands or compositions of the invention. The ligands according to the method of the present invention may be employed in in vivo therapeutic and prophylactic applications, in vivo diagnostic applications and the like.

Therapeutic and prophylactic uses of ligands of the invention involve the administration of ligands according to the invention to a recipient mammal, such as a human. The ligands bind to targets with great avidity. In some embodiments, the ligands can allow the cross-linking of two targets, for example in recruiting cytotoxic T-cells to mediate the killing of tumor cell lines.

Substantially pure ligands, for example ds-dAbs, of at least 90 to 95% homogeneity are preferred for administration to a mammal, and 98 to 99% or more homogeneity is most preferred for pharmaceutical uses, especially when the mammal is a human. Once purified, partially or to homogeneity as desired, the ligands may be used diagnostically or therapeutically (including extracorporeally) or in developing and performing assay procedures, immunofluorescent stainings and the like (Lefkovite and Pernis, (1979 and 1981) Immunological Methods, Volumes I and II, Academic Press, NY).

For example, the ligands, of the present invention will typically find use in preventing, suppressing or treating disease states. For example, ligands can be administered to treat, suppress or prevent a chronic inflammatory disease, allergic hypersensitivity, cancer, bacterial or viral infection, autoimmune disorders (which include, but are not limited to, Type I diabetes, asthma, multiple sclerosis, rheumatoid arthritis, juvenile rheumatoid arthritis, psoriatic arthritis, spondylarthropathy (e.g., ankylosing spondylitis), systemic lupus erythematosus, inflammatory bowel disease (e.g., Crohn's disease, ulcerative colitis), myasthenia gravis and Behcet's syndrome), psoriasis, endometriosis, and abdominal adhesions (e.g., post abdominal surgery).

The ligands are particularly useful for treating infectious diseases in which cells infected with an infectious agent contain higher levels of cell surface targets than uninfected cells, or that contain one or more cell surface targets that are not present on infected cells, such as a protein that is encoded by the infectious agent (e.g., bacteria, virus).

Ligands according to the invention that are able to bind to extracellular targets can be endocytosed, and can deliver therapeutic agents (e.g., a toxin) intracellularly (e.g., deliver a dAb that binds an intracellular target). In addition, ligands, provide a means by which each binding domain (e.g., a dAb monomer) that is specifically able to bind to an intracellular target can be delivered to an intracellular environment. This strategy requires, for example, a binding domain with physical properties that enable it to remain functional inside the cell. Alternatively, if the final destination intracellular compartment is oxidising, a well folding ligand may not need to be disulphide free.

In the instant application, the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest. Treatment includes ameliorating symptoms associated with the disease, and also preventing or delaying the onset of the disease and also lessening the severity or frequency of symptoms of the disease.

The terms “cancer” refer to or describe the physiological condition in mammals that is typically characterized by dysregulated cellular proliferation or survival. Examples of cancer include, but are not limited to, carcinoma, lymphoma, blastoma, sarcoma, and leukemia and lymphoid malignancies. More particular examples of cancers include squamous cell cancer (e.g. epithelial squamous cell cancer), lung cancer (e.g., small-cell lung carcinoma, non-small cell lung cancer, adenocarcinoma of the lung, squamous carcinoma of the lung), cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, multiple myeloma, chronic myelogenous leukemia, acute myelogenous leukemia, endometrial or uterine carcinoma, salivary gland carcinoma, kidney or renal cancer, prostate cancer, vulval cancer, thyroid cancer, hepatic carcinoma, anal carcinoma, penile carcinoma, head and neck cancer, and the like.

Animal model systems which can be used to assess efficacy of the ligands of the invention in preventing treating or suppressing disease (e.g., cancer) are available. Suitable models of cancer include, for example, xenograft and orthotopic models of human cancers in animal models, such as the SCID-hu myeloma model (Epstein J, and Yaccoby, S., Methods Mol Med. 113:183-90 (2005), Tassone P, et al., Clin Cancer Res. 11 (11):4251-8 (2005)), mouse models of human lung cancer (e.g., Meuwissen R and Berns A, Genes Dev. 19(6):643-64 (2005)), and mouse models of metastatic cancers (e.g., Kubota T., J Cell Biochem. 56(1):4-8 (1994)).

Generally, the present ligands will be utilized in purified form together with pharmacologically appropriate carriers. Typically, these carriers include aqueous or alcoholic/aqueous solutions, emulsions or suspensions, including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition). A variety of suitable formulations can be used, including extended release formulations.

The ligand of the present invention may be used as separately administered compositions or in conjunction with other agents. The ligands can be administered and or formulated together with one or more additional therapeutic or active agents. When a ligand is administered with an additional therapeutic agent, the ligand can be administered before, simultaneously with, or subsequent to administration of the additional agent. Generally, the ligand and additional agent are administered in a manner that provides an overlap of therapeutic effect. Additional agents that can be administered or formulated with the ligand of the invention include, for example, various immunotherapeutic drugs, such as cylcosporine, methotrexate, adriamycin or cisplatinum, antibiotics, antimycotics, anti-viral agents and immunotoxins. For example, when the antagonist is administered to prevent, suppress or treat lung inflammation or a respiratory disease, it can be administered in conduction with phosphodiesterase inhibitors (e.g., inhibitors of phosphodiesterase 4), bronchodilators (e.g., beta2-agonists, anticholinergerics, theophylline), short-acting beta-agonists (e.g., albuterol, salbutamol, bambuterol, fenoterol, isoetherine, isoproterenol, levalbuterol, metaproterenol, pirbuterol, terbutaline and tornlate), long-acting beta-agonists (e.g., formoterol and salmeterol), short-acting anticholinergics (e.g., ipratropium bromide and oxitropium bromide), long-acting anticholinergics (e.g., tiotropium), theophylline (e.g. short-acting formulation, long acting formulation), inhaled steroids (e.g., beclomethasone, beclomethasone, budesonide, flunisolide, fluticasone propionate and triamcinolone), oral steroids (e.g., methylprednisolone, prednisolone, prednisolon and prednisone), combined short-acting beta-agonists with anticholinergics (e.g., albuterol/salbutamol/ipratopium, and fenoterol/ipratopium), combined long-acting beta-agonists with inhaled steroids (e.g., salmeterol/fluticasone, and formoterol/budesonide) and mucolytic agents (e.g., erdosteine, acetylcysteine, bromheksin, carbocysteine, guiafenesin and iodinated glycerol.

The ligands of the invention can be coadministered (e.g., to treat cancer) with a variety of suitable co-therapeutic agents, including cytokines, analgesics/antipyretics, antiemetics, and chemotherapeutics.

Suitable co-therapeutic agents include cytokines, which include, without limitation, a lymphokine, tumor necrosis factors, tumor necrosis factor-like cytokine, lymphotoxin, interferon, macrophage inflammatory protein, granulocyte monocyte colony stimulating factor, interleukin (including, without limitation, interleukin-1, interleukin-2, interleukin-6, interleukin-12, interleukin-15, interleukin-18), growth factors, which include, without limitation, (e.g., growth hormone, insulin-like growth factor 1 and 2 (IGF-1 and IGF-2), granulocyte colony stimulating factor (GCSF), platelet derived growth factor (PGDF), epidermal growth factor (EGF), and agents for erythropoiesis stimulation, e.g., recombinant human erythropoietin (Epoetin alfa), EPO, a hormonal agonist, hormonal antagonists (e.g., flutamide, tamoxifen, leuprolide acetate (LUPRON)), and steroids (e.g., dexamethasone, retinoid, betamethasone, cortisol, cortisone, prednisone, dehydrotestosterone, glucocorticoid, mineralocorticoid, estrogen, testosterone, progestin).

Analgesics/antipyretics can include, without limitation, aspirin, acetaminophen, ibuprofen, naproxen sodium, buprenorphine hydrochloride, propoxyphene hydrochloride, propoxyphene napsylate, meperidine hydrochloride, hydromorphone hydrochloride, morphine sulfate, oxycodone hydrochloride, codeine phosphate, dihydrocodeine bitartrate, pentazocine hydrochloride, hydrocodone bitartrate, levorphanol tartrate, diflunisal, trolamine salicylate, nalbuphine hydrochloride, mefenamic acid, butorphanol tartrate, choline salicylate, butalbital, phenyltoloxamine citrate, diphenhydramine citrate, methotrimeprazine, cinnamedrine hydrochloride, meprobamate, and the like.

Antiemetics can also be coadministered to prevent or treat nausea and vomiting, e.g., suitable antiemetics include meclizine hydrochloride, nabilone, prochlorperazine, dimenhydrinate, promethazine hydrochloride, thiethylperazine, scopolamine, and the like.

Chemotherapeutic agents, as that term is used herein, include, but are not limited to, for example antimicrotubule agents, e.g., taxol (paclitaxel), taxotere (docetaxel); alkylating agents, e.g., cyclophosphamide, carmustine, lomustine, and chlorambucil; cytotoxic antibiotics, e.g., dactinomycin, doxorubicin, mitomycin-C, and bleomycin; antimetabolites, e.g., cytarabine, gemcitatin, methotrexate, and 5-fluorouracil; antimiotics, e.g., vincristine vinca alkaloids, e.g., etoposide, vinblastine, and vincristine; and others such as cisplatin, dacarbazine, procarbazine, and hydroxyurea; and combinations thereof.

The ligands of the invention can be used to treat cancer in combination with another therapeutic agent. For example, a ligand of the invention can be administered in combination with a chemotherapeutic agent. Advantageously, in such a therapeutic approach, the amount of chemotherapeutic agent that must be administered to be effective can be reduced. Thus the invention provides a method of treating cancer comprising administering to a patient in need thereof a therapeutically effective amount of a ligand of the invention and a chemotherapeutic agent, wherein the chemotherapeutic agent is administered at a low dose. Generally the amount of chemotherapeutic agent that is coadministered with a ligand of the invention is about 80%, or about 70%, or about 60%, or about 50%, or about 40%, or about 30%, or about 20%, or about 10% or less, of the dose of chemotherapeutic agent alone that is normally administered to a patient. Thus, cotherapy is particularly advantageous when the chemotherapeutic agent causes deleterious or undesirable side effects that may be reduced or eliminated at a lower dose.

Pharmaceutical compositions can include “cocktails” of various cytotoxic or other agents in conjunction with ligands of the present invention, or even combinations of ligands according to the present invention having different specificities, such as ligands selected using different target antigens or epitopes, whether or not they are pooled prior to administration.

The route of administration of pharmaceutical compositions according to the invention may be any suitable route, such as any of those commonly known to those of ordinary skill in the art. For therapy, including without limitation immunotherapy, the ligands of the invention can be administered to any patient in accordance with standard techniques. The administration can be by any appropriate mode, including parenterally, intravenously, intramuscularly, intraperitoneally, transdermally, intrathecally, intrarticularly, via the pulmonary route, or also, appropriately, by direct infusion (e.g., with a catheter). The dosage and frequency of administration will depend on the age, sex and condition of the patient, concurrent administration of other drugs, counterindications and other parameters to be taken into account by the clinician. Administration can be local (e.g., local delivery to the lung by pulmonary administration, (e.g., intranasal administration) or local injection directly into a tumor) or systemic as indicated.

The ligands of this invention can be lyophilised for storage and reconstituted in a suitable carrier prior to use. This technique has been shown to be effective with conventional immunoglobulins and art-known lyophilisation and reconstitution techniques can be employed. It will be appreciated by those skilled in the art that lyophilisation and reconstitution can lead to varying degrees of antibody activity loss (e.g. with conventional immunoglobulins, IgM antibodies tend to have greater activity loss than IgG antibodies) and that use levels may have to be adjusted upward to compensate.

The compositions containing the ligands can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's health, but generally range from 0.005 to 5.0 mg of ligandper kilogram of body weight, with doses of 0.05 to 2.0 mg/kg/dose being more commonly used. For prophylactic applications, compositions containing the present ligands or cocktails thereof may also be administered in similar or slightly lower dosages, to prevent, inhibit or delay onset of disease (e.g., to sustain remission or quiescence, or to prevent acute phase). The skilled clinician will be able to determine the appropriate dosing interval to treat, suppress or prevent disease. When a ligand is administered to treat, suppress or prevent a disease, it can be administered up to four times per day, twice weekly, once weekly, once every two weeks, once a month, or once every two months, at a dose of, for example, about 10 μg/kg to about 80 mg/kg, about 100 μg/kg to about 80 mg/kg, about 1 mg/kg to about 80 mg/kg, about 1 mg/kg to about 70 mg/kg, about 1 mg/kg to about 60 mg/kg, about 1 mg/kg to about 50 mg/kg, about 1 mg/kg to about 40 mg/kg, about 1 mg/kg to about 30 mg/kg, about 1 mg/kg to about 20 mg/kg, about 1 mg/kg to about 10 mg/kg, about 10 μg/kg to about 10 mg/kg, about 10 μg/kg to about 5 mg/kg, about 10 μg/kg to about 2.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg or about 10 mg/kg. In particular embodiments, the dual-specific ligand is administered to treat, suppress or prevent a chronic inflammatory disease once every two weeks or once a month at a dose of about 10 μg/kg to about 10 mg/kg (e.g., about 10 μg/kg, about 100 μg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg or about 10 mg/kg.)

In particular embodiments, the ligand of the invention is administered at a dose that provides for selective binding to double positive cells in vivo. As described herein selective binding to double positive cells can be achieved when the ligand is used at a concentration of about 1 pM to about 150 nM. A dose that is sufficient to achieve a serum concentration of ligand that is from about 1 pM to about 150 nM can be administered. The skilled physician can determine appropriate dosing to achieve such a serum concentration, for example by titrating ligand and monitoring the serum concentration of ligand. Therapeutic regiments that involve administering a therapeutic agent to achieve a desired serum concentration of agent are common in the art, particularly in the field of oncology.

Treatment or therapy performed using the compositions described herein is considered “effective” if one or more symptoms are reduced (e.g., by at least 10% or at least one point on a clinical assessment scale), relative to such symptoms present before treatment, or relative to such symptoms in an individual (human or model animal) not treated with such composition or other suitable control. Symptoms will obviously vary depending upon the disease or disorder targeted, but can be measured by an ordinarily skilled clinician or technician. Such symptoms can be measured, for example, by monitoring the level of one or more biochemical indicators of the disease or disorder (e.g., levels of an enzyme or metabolite correlated with the disease, affected cell numbers, etc.), by monitoring physical manifestations (e.g., inflammation, tumor size, etc.), or by an accepted clinical assessment scale, for example, the Expanded Disability Status Scale (for multiple sclerosis), the Irvine Inflammatory Bowel Disease Questionnaire (32 point assessment evaluates quality of life with respect to bowel function, systemic symptoms, social function and emotional status—score ranges from 32 to 224, with higher scores indicating a better quality of life), the Quality of Life Rheumatoid Arthritis Scale, or other accepted clinical assessment scale as known in the field. A sustained (e.g., one day or more, preferably longer) reduction in disease or disorder symptoms by at least 10% or by one or more points on a given clinical scale is indicative of “effective” treatment. Similarly, prophylaxis performed using a composition as described herein is “effective” if the onset or severity of one or more symptoms is delayed, reduced or abolished relative to such symptoms in a similar individual (human or animal model) not treated with the composition.

A composition containing ligands according to the present invention may be utilized in prophylactic and therapeutic settings to aid in the alteration, inactivation, killing or removal of a select target cell population in a mammal. In addition, the ligands and selected repertoires of polypeptides described herein may be used extracorporeally or in vitro selectively to kill, deplete or otherwise effectively remove a target cell population from a heterogeneous collection of cells. Blood from a mammal may be combined extracorporeally with the ligands, e.g., antibodies, cell-surface receptors or binding proteins thereof whereby the undesired cells are killed or otherwise removed from the blood for return to the mammal in accordance with standard techniques.

### EXAMPLES

In the examples described herein, CD38 is also referred to as DOM11, CD138 is also referred to as DOM12, CEA is also referred to as DOM13, and CD56 is also referred to as DOM14.

Selections and Screening of dAbs that Bind CD38, CD138, CEA or CD56

dAbs were selected using antigens that were expressed as Fc-fusion proteins in mammalian cells. Three rounds of selection were performed using dAb libraries for CD38, CD138, CEA and CD56 captured alternately on protein G (Dynal) and anti-human Fc (Novagen) magnetic beads. Selection outputs were tested in ELISA for specificity as phage and as soluble dAbs at rounds 2 and 3 on cognate antigen but not on non-cognate antigen. For soluble ELISAs all Vk dAbs were cross linked with protein L. For each antigen the soluble ELISA positive clones were sequenced showing the selections to have diverse outputs.

### Binding Assays to Determine dAb Positive Clones

ELISA positive clones were expressed in 50 ml cultures and purified on protein A (VH clones) or protein L (Vk clones) as appropriate. Briefly, a phage expression plasmid (pDOM5) encoding the dAb was transformed into HB2151 E. coli and the cells were plated onto TYE plates containing 50 μg/ml carbenicillin and 5% glucose and incubated overnight at 37° C. The expression of the dAb into the culture supernatant was made using auto-induction according to the following method: the following components were added to a 250 ml baffled flask: 50 ml of TB, 100 μg/ml carbenicillin, 1 drop of antifoam A204 (Sigma), 1 ml Solution 1, 2.5 ml Solution 2 and 0.05 ml Solution 3 from the Novagen Overnight Express Autoinduction Kit and a single colony from the transformed E. coli cells. The flasks were covered with Milliwrap PTFE membrane and the culture allowed to grow and express protein for 48 his at 250 rpm at 30° C. The protein was purified directly from the culture supernatant using protein A or L.

All dAbs were analysed by FACS on antigen positive and negative cell lines using the following method.

The determination of cell binding by FACS was carried out as follows: cells were centrifuged at 250 g for 5 minutes and the growth medium was removed. The cells were resuspended in FACS incubation buffer at 4° C. at a density of 2×10⁶ cells/ml. The cells were blocked by incubating for 15 minutes at 4° C. in FACS incubation buffer. Fifty microliters of 2× stock of primary antibody (anti-CD38 FITC, anti-CD138 FITC or mIgG1 FITC conjugated isotype control (all BD Biosciences) was added; or dAb was added to cells in FACS incubation buffer and incubated for 30-60 minutes at 4° C. The cells were then washed once in FACS incubation buffer. One hundred microliters of secondary antibody (rabbit anti-Vk) was added to cells in FACS incubation buffer and incubated 30-60 minutes at 4° C. The cells were washed once in FACS incubation buffer. Then 100 ul of 1× tertiary antibody was added to the cells in FACS incubation buffer and incubated for 30-60 mins at 4° C. (for dAbs the tertiary antibody is anti-rabbit FITC (Sigma)). The cells were washed twice in FACS incubation buffer. The cell pellet was resuspend in 200 ul FACS incubation buffer+viable cell marker (BD Viaprobe). The cells were then analyzed by flow cytometry.

The cell lines described in Table 3 were used for FACS analysis. The phentypes of the cell lines were determined by FACS. Suitable cells that have a suitable phenotype for assessing binding specificity of the ligands can be obtained from cell depositories such as American Type Culture Collection (e.g., accession numbers CCL-155, CRL 9068, CCL-86, CRL1929, TIB 196, CRL 1730, CRL2408, HTB 173, HTB 119, CRL 5834) and Deutsche Sammlung von Mikroorganismen und Zellkulturen GmbH (e.g., accession numbers ACC50, ACC 31).

TABLE 3
Phenotype of cell lines used in FACS analysis

| | Phenotype determined by |
|---|---|
| Cell line | FACS |
| RPMI8226 | CD138+ |
| | CD31− |
| | CD38+ |
| | CD56+ |
| OPM2 | CD138+ |
| | CD38+ |
| NCIH929 | CD138+ |
| | CD31− |
| | CD38+ |
| | CD56+ |
| RAJI | CD83+ |
| | CD138− |
| SUPB15 | CD138− |
| | CD31+ |
| | CD38+ |
| | CD56− |
| U266 | CD138+ |
| | CD31+ (low expression) |
| | CD38+ (low expression) |
| | CD56− |
| Huvec | CD138+ |
| | CD31+ |
| | CD38− |
| | CD56− |
| CCRF-CEM | CD38− |
| | CD138− |
| | CEA− |
| | CD56− |
| K299 | CD38− |
| | CD138− |
| | CD56− |
| | CEA− |
| NK92MI | CEA− |
| | CD56+ |
| NCI-H146 | CEA(very weakly +ve) |
| | CD56+ |
| NCI-H69 | CEA+ |
| | CD56+ |
| NCI-H647 | CEA− |
| | CD56− |
| | NCA+ |
| | CD138+ |

### Results

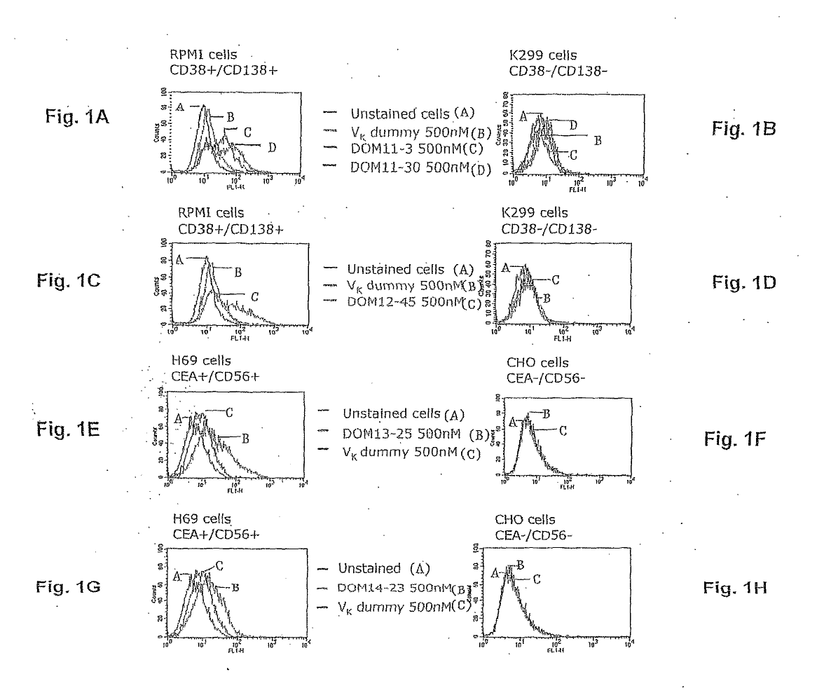

In this study, the dAbs DOM11-3, DOM11-30, DOM12-45, DOM13-25 and DOM14-23 were identified by FACS analysis as having good binding characteristics for CD36, CD38, CD138, CEA and CD56 respectively. See FIGS. 1A-1H.

TABLE 4
Propertis of anti-CD38 and anti-CD138
dAbs properties by FACS analysis

| | RPMI | SUPB | HUVEC | K299 |
|---|---|---|---|---|
| | CD38+/ | CD38+/ | CD38−/ | CD38−/ |
| | 138+ | CD138− | CD138+ | 138− |
| Anti-CD38 | | | | |
| DOM11-3 | ✓ | ✓ | X | X |
| DOM11-7 | ✓ | ✓ | X | X |
| DOM11-23 | ✓ | | | |
| DOM11-24 | ✓ | | | X |
| DOM11-30 | ✓ | | | X |
| DOM11-32 | ✓ | | | X |
| DOM11-37 | ✓ | | | |
| DOM11-38 | ✓ | | | X |
| DOM11-39 | ✓ | | | X |
| Anti-CD138 | | | | |
| DOM12-17 | ✓ | | ✓ | X |
| DOM12-26 | ✓ | | ✓ | (✓) |
| DOM12-45 | ✓ | | ✓ | X |

✓ = dAb binds
X = dAb does not bind

### BIACORE Analysis

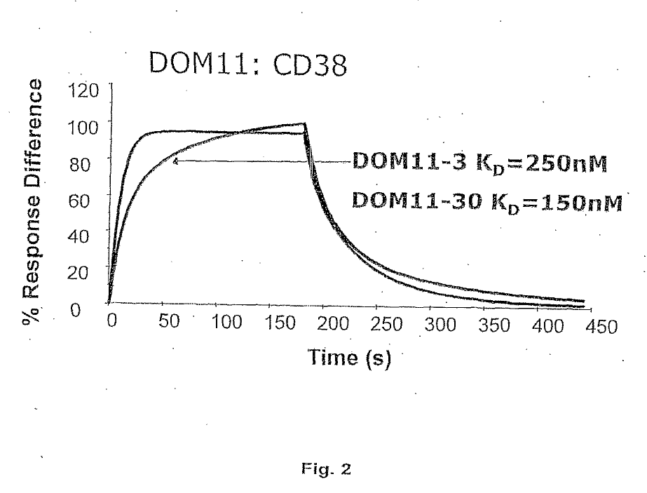

Anti-CD38, anti-CEA and anti-CD56 dAbs that were identified as FACS positive clones were in addition analysed by Biacore using the following procedure. The CM5 chip surface was activated by flushing 1:1 EDC/NHS (0.4M1-ethyl-3-(3-dimenthylaminopropyl)-carbodiimide in water; 0.1 M N-hydroxysuccinimide in water) at a flow rate of 5 uL/min for 10 minute contact time. CD38 was immobilised at 500 nM in Acetate buffer pH4 at 5 uL/min this was repeated until the RUs reached between 500 and 1000 (low density). CEA and CD56 were coupled in acetate buffer pH 4.5. Any excess reactive groups were deactivated by running 1M ethanolamine-HCl over the CM5 chip (again 5 uL/min for 7 mins). The affinities of the anti-CD38, anti-CEA and anti-CD56 dAbs were measured on the biacore as described above. For each target, dAbs were found that bound with an affinity in the 100-200 nM range. FIG. 2 shows the results from two anti-CD38 dAbs (DOM11-30 and DOM11-3) that were measured for affinity of the Biacore. DOM11-30 had an affinity (K_{D}) of 150 nM and DOM11-2 had an affinity of 250 nM.

Epitope Mapping Anti-CD38 dAbs

Epitope mapping was performed to determine whether anti-CD38 dAbs bound to different epitopes on CD38. The assay was performed on BIAcore as described above using a chip coated at medium density (RUs of ˜2000). CD38 was coated on to a CM5 chip at medium density as described above. Using the co-inject function, the first anti-CD38 dAbs was injected at a concentration of 500 nM. Both the first and second anti-CD38 dabs were the co-injected at the same concentration (500 nM). As both dAbs bind different epitopes, the RUs during the second injection increase beyond the level of binding of the first dAb.

The results showed that anti-CD38 dAbs DOM113, DOM11-30 and DOM11-23 bind to different epitopes on CD38. See FIGS. 3A-3D.

TABLE 5
Properties of anti-CEA dAbs

| | | | | H647 |
|---|---|---|---|---|
| dAb | LS174-T | H69 | biacore | (CEA−/NCA+) |
| DOM13-25 | ++ | ++ | Affinity 100- | − |
| | | | 200 nM | |
| DOM13-57 | − | + | NT | − |
| | | (very weak) | | |
| DOM13-58 | + | + | binds-low | − |
| | (weak) | | affinity | |
| DOM13-59 | + | + | Affinity 400- | − |
| | (weak) | | 800 nM | |
| DOM13-64 | NT | NT | binds-low | − |
| | | | affinity | |
| DOM13-65 | + | + | binds-low | − |
| | | | affinity | |
| DOM13-74 | + | + | Affinity 100- | − |
| | | | 200 nM | |
| DOM13-93 | + | + | binds-low | − |
| | | | affinity | |
| DOM13-95 | + | + | binds-low | − |
| | | | affinity | |

++ strong binding
+ binds
− does not bind
NT not tested

TABLE 6
Properties of anti-CD56 dAbs

| dAb | H82 | H69 | biacore |
|---|---|---|---|
| DOM14-23 | + | ND | Affinity 100-200 nM |
| | (as dimer) | | |
| DOM14-48 | − | ++ | binds-low affinity |
| DOM14-56 | − | ~+ | binds-low affinity |
| DOM14-57 | − | + | does not bind |
| DOM14-62 | − | ~+ | does not bind |
| DOM14-63 | − | ++ | Affinity 100 nM |
| DOM14-68 | + | ++ | does not bind |
| DOM14-70 | + | + | Affinity 500-800 nM |

++ strong binding
+ binds
− does not bind

Ligands that contain an anti-CD38 dAb and an anti-CD138 dAb Low affinity dAbs have been identified that bind CD38 or CD138. These dAbs have been linked by in line fusion to create dual specific dAbs (ligands) that bind specifically to antigen expressing cells by FACS. All dAbs were expressed in E. coli and purified using protein L agarose followed by Resource S cation exchange chromatography when required.

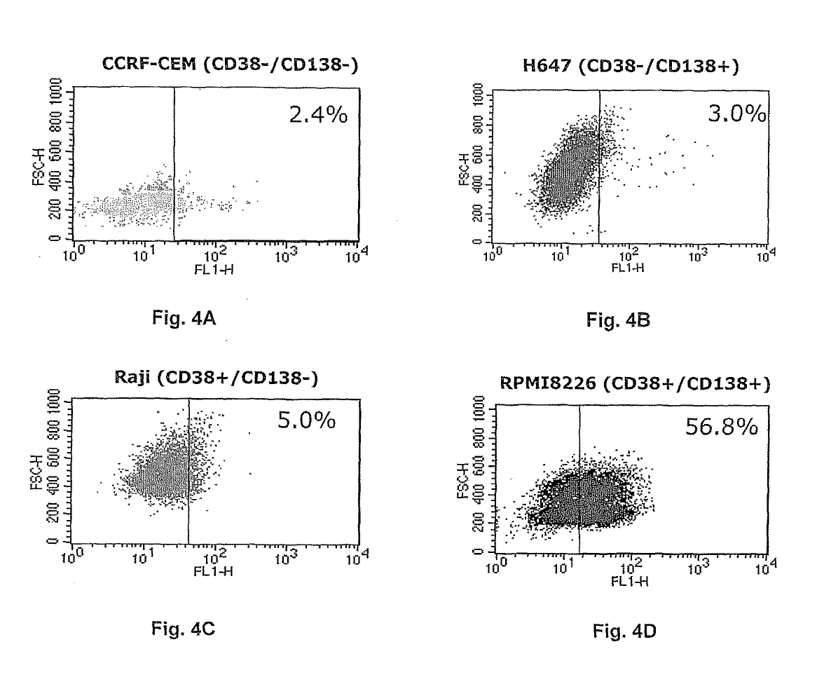

All dAbs have been shown to bind as monomers to antigen expressing cell lines but not to antigen negative cell lines. Anti-CD38 dAbs and anti-CD138 dAbs have been paired as in-line fusions and examined for binding by FACS on double positive and negative cell lines as described above. The optimum dual specific dAb pairings were DOM11-3/DOM12-45 and DOM11-30/DOM12-45. At the optimum concentration (25-50 nM), these pairing bound strongly to double positive cell lines (CD38+/CD138+) but not to single positive or negative cell lines. See FIGS. 4A-4D.

### Internalization

### Method

Cells were washed once in RPMI1640+10% FCS (Internalization buffer). The cell pellet was resuspended in required volume of internalization buffer and divided between appropriate number of tubes (50 μl per tube). The cells were incubated for 15 minutes. in internalization buffer to block. Then 50 ul of 2× stock of pre-mixed primary and secondary antibodies (dAb+rabbit anti-Vk) were added to cells in internalization buffer and incubated for 60 minutes at 4° C. The cells were washed once in internalization buffer. 100 μl 1× tertiary antibody (anti rabbit FITC) was added to cells in internalization buffer and incubated for 30-60 minutes at 4° C.

The cells were washed once in internalization buffer. The relevant samples were incubated at 37° C. for 1.5 hours to allow internalization. Two sets of duplicate samples were maintained at 4° C. polypeptide.

To differentiate between surface bound and internalized dAbs, a sample of cells that had been incubated at 4° C. only and the cells that have been incubated at 37° C. were acid washed, removing cell surface dAb only. The cells were then washed twice in acid wash buffer then twice in PBS. The cells were resuspended in 200 ul PBS+10 ul BD Viaprobe and were analyzed by flow cytometry. The proportion of cells labeled and 4° C. only (cell surface bound) compared with 37° C. with acid wash treatment (internalized) was assessed by FACS. Alternatively for confocal microscopy the cells are fixed in 4% paraformaldehyde solution and mounted onto coverslips.

### Results

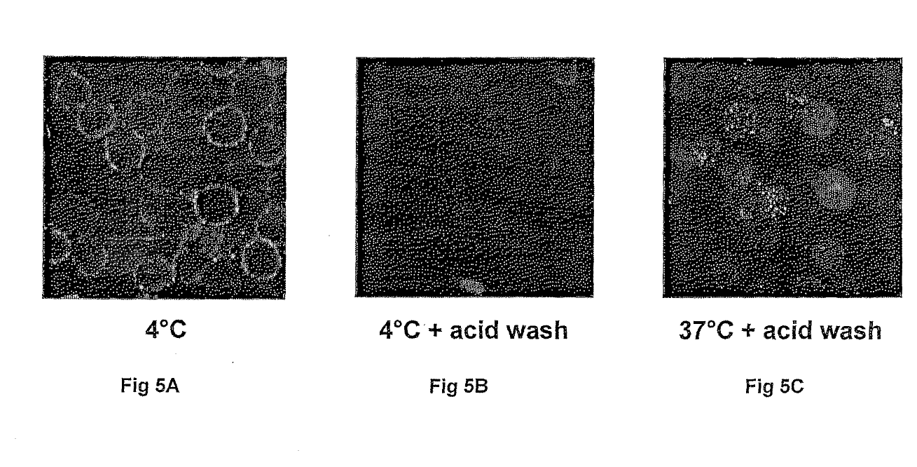

Both anti-CD38/anti-CD138 dual-specific ligands (DOM11-3/DOM12-45 and DOM11-30/DOM12-45) were shown to internalize on the CD38+ cell line Raji by FACS and confocal microscopy. FIGS. 5A-5C show that CD38 positive cell line was labeled with DOM11-3/DOM12-45 (500 nM, and visualized with FITC staining on a Zeiss LSM510 META confocal microscope). Internalisation was revealed as acid resistance fluorescence at 37° C.

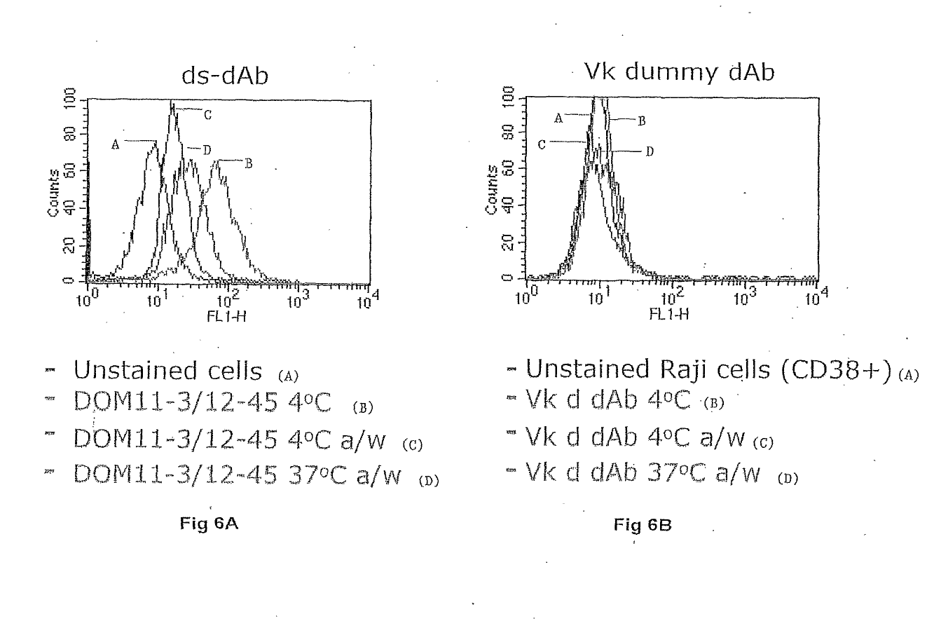

Anti-CD38/anti-CD138 dual-specific ligands, DOM11-3/DOM12-45 and DOM11-30/DOM12-45, have also been shown to internalize on the dual expressing multiple myeloma cell line OPM2 (DSMZ ACC50). See FIGS. 6A and 6B.

TABLE 7
A determination of the proportion of internalized dual specific dAbs.

| | DOM11-3/ | DOM11-30/ | DOM14-23/ | |
|---|---|---|---|---|
| | DOM12-45 | DOM12-45 | DOM12-45 | Vk dummy |
| % internalized | 76% | 8% | 43% | 0.2% |

### Intracellular Localization

In this study the intracellular localization of the internalized dual specific dAbs was investigated.

### Method

Briefly, the intracellular localization of the internalized dual specific dAbs (ds-dAbs) was investigated. dAbs internalized by Raji (CD38+) cells as described above have been counterstained with magic red according to manufacturer's instruction (serotec). Magic Red is a marker for Cathepsin B which localizes to the lysosomal compartment. Both DOM11-30/DOM12-45 and DOM11-3/DOM12-45 have shown co-localization with this marker.

### Results

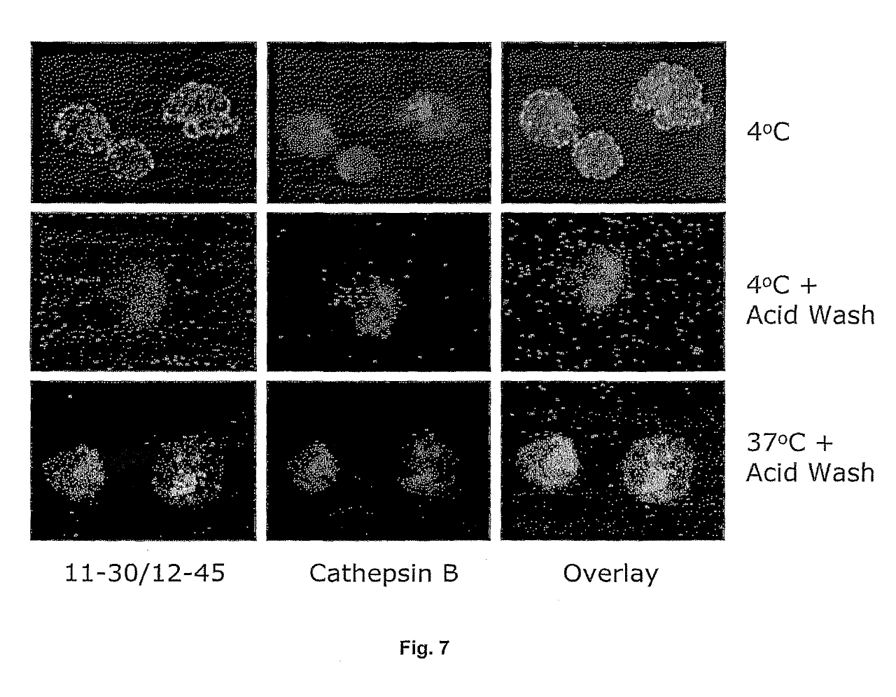

FIG. 7 shows co-localization of CD38/CD138 with the lysosomal marker, Cathepsin B, on Raji Cells, visualized by confocal microscopy. Both DOM11-30/DOM12-45 and DOM11-3/DOM12-45 have shown co-localization with this marker.

These results show that a ligand can be internalized to the lysosomal compartment, where the ligand can be processed, e.g., by proteolytic cleavage (cathepsin B cleavage) to, for example, release a toxin.

### Dual Specific Ligand-Poly Ethylene Glycol (PEG) Conjugates Method

Anti CD38/anti CD138 dual specific ligands, DOM11-3/DOM12-45 and DOM11-30/DOM12-45, were pegylated via a c-terminal cysteine residue with either 5K, 20K, 30K or 40K PEGs. The engineered cysteine at the c-terminus of the dAb allows the site-specific attachment of MAL-PEG.

Glycerol was added to the dAb protein solution to a final concentration of 20% (v/v) and dithiothreitol to 5 mM. The solution was incubated at room temperature for 20 minutes to allow the reduction of the surface thiol. The volume of the sample was reduced to 2.5 ml by using a centrifugal concentrator (Vivascience) (4,500 rpm). The protein solution was buffer exchanged to remove the reducing agent using a PD-10 column (Amersham). The PD-10 column was equilibrated with 25 mls of coupling buffer (20 mM BIS-Tris pH 6.5, 5 mM EDTA and 10% glycerol [v/v]), before the 2.5 ml of reduced protein was applied. The protein solution was allowed to completely enter the resin bed before eluting the dAb by the addition of a further 3.5 ml of coupling buffer. The protein was then immediately coupled. The protein concentration (mg/ml) was determined by measuring the absorbance at 280 nm. The protein amount was converted from mg/ml to a molar concentration. A three molar excess of the MAL-PEG was added. The reaction was allowed to proceed overnight at room temperature. The sample was buffer exchanged using a PD-10 desalting column to remove uncoupled MAL-PEG. FACS analysis of the pegylated samples was carried out as described above for binding and internalization of dAbs.

### Results

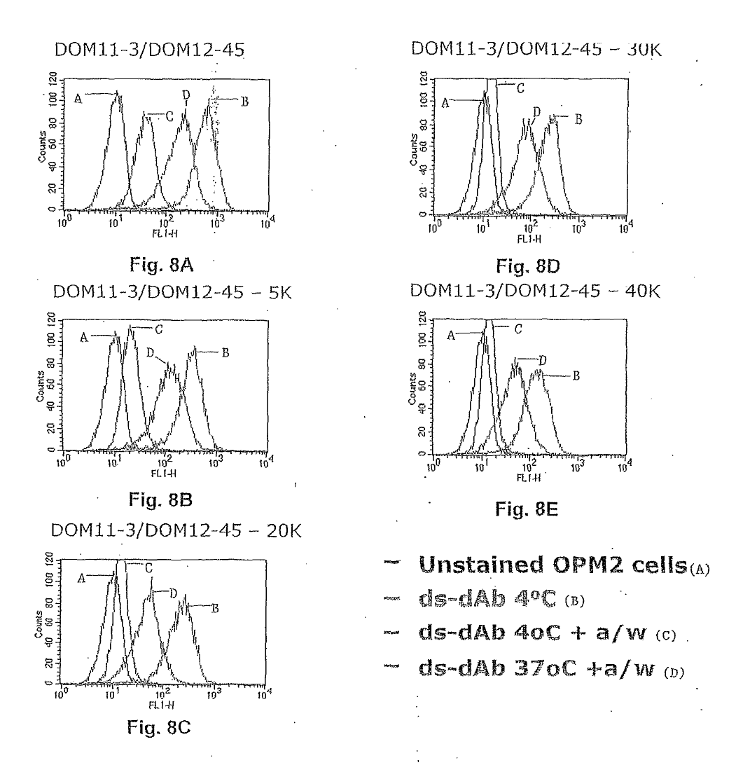

The results show that when pegylated, dual specific ligands bind to their targets to a similar extent to non-pegylated dual specific ligands. Some reduction in binding was seen, in particular with the larger PEGs for anti-CD38/anti-CD138 dual-specific ligands, DOM1-30/DOM12-45. In addition pegylated forms of anti-CD38 (DOM11) were internalized by OPM2 multiple myeloma cells to a similar extent as the non-pegylated ligand (See FIGS. 8A-8E).

### Anti-CD38/Anti-CD138 Dual Specific Ligand-Toxin Conjugate

### Preparation of Anti-CD38/Anti-CD138, (DOM11-3/DOM12-45) Dual-Specific Ligands

An Anti-CD38/anti-CD138 (DOM11-3/DOM12-45) dual-specific ligand was expressed in E. coli and purified using protein L agarose followed by Resource S cation exchange chromatography. Vk dummy/Vk dummy homodimer was also expressed and purified for use as a negative control.

Conjugation of Toxin-selenium to anti-CD38/anti-CD138 (DOM11-3/12-45)

Selenium was conjugated to the anti-CD38/anti-CD138 dual-specific ligand using a 3 carbon acid or a 3 carbon amine linker. (See, U.S. Pat. No. 5,783,454, the teachings of which are incorporated herein by reference.) On average, two selenium molecules were coupled to each anti-CD38/anti-CD138 dual-specific ligand.

Internalization of Se conjugated Dual Specific Ligands

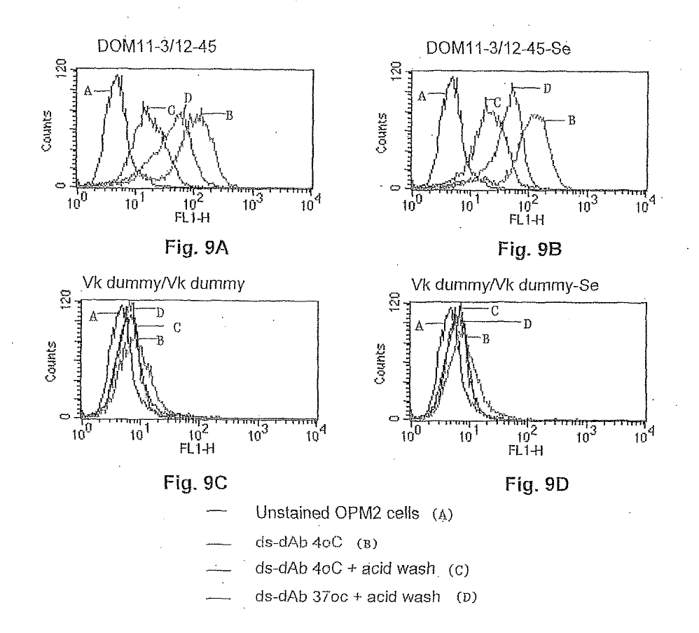

Internalisation of the Se conjugated dAbs by OPM2 cells was examined by FACS as described above. Selenium conjugated anti-CD38/anti-CD138 (DOM11-3/DOM12-45) dual specific ligand was internalized to the same degree as unconjugated dAbs, whereas Vk dummy dAb either un-conjugated or conjugated with selenium were not internalized. See FIGS. 9A-9D.

Anti-CD38/Anti-CD138 Cell killing, Assays

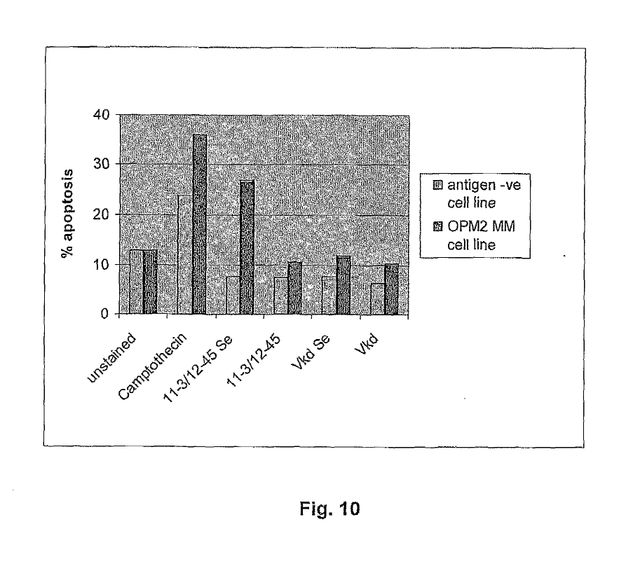

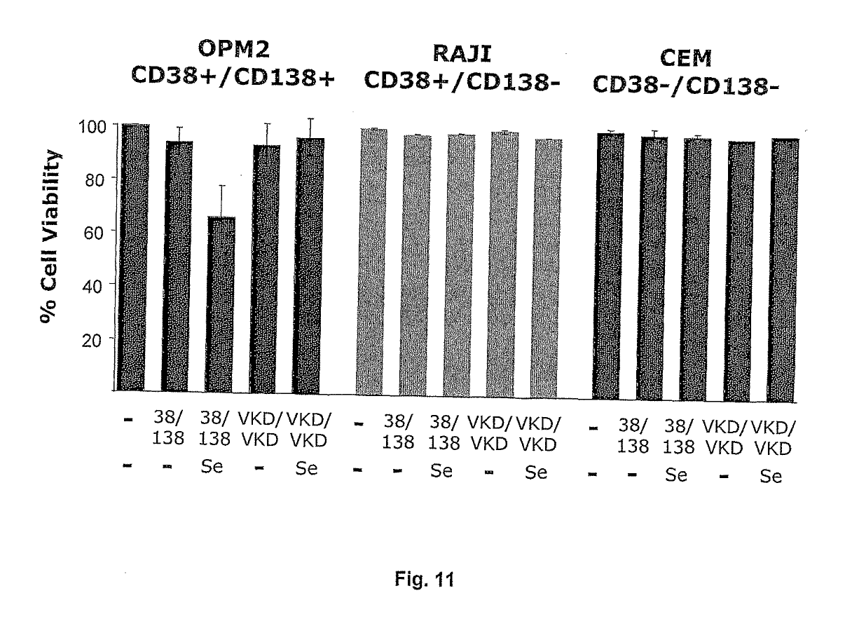

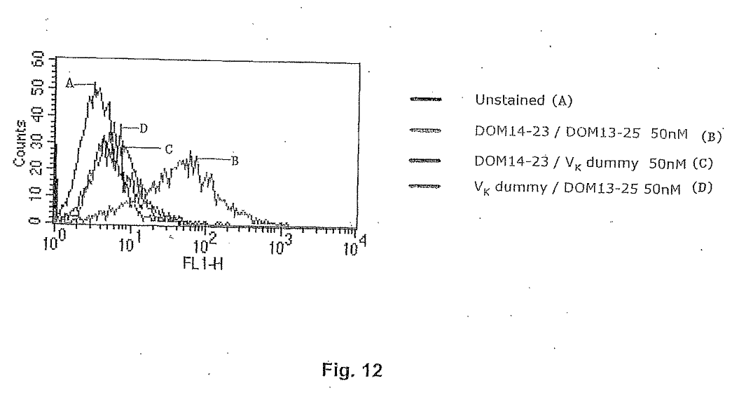

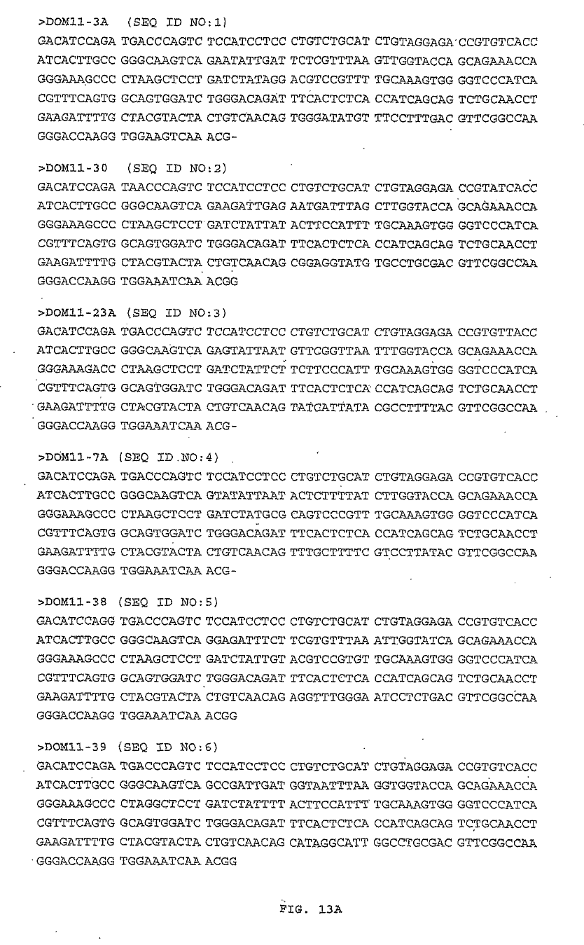

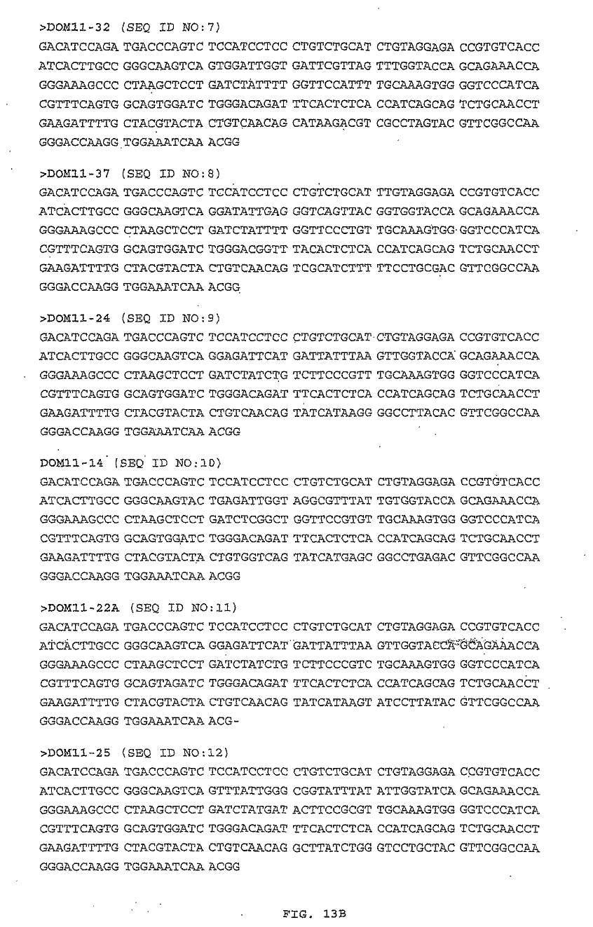

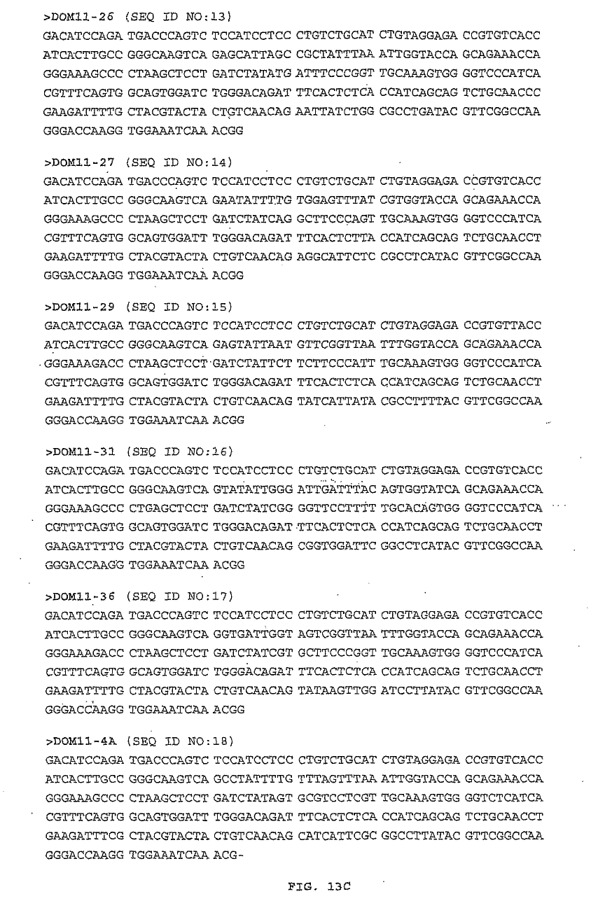

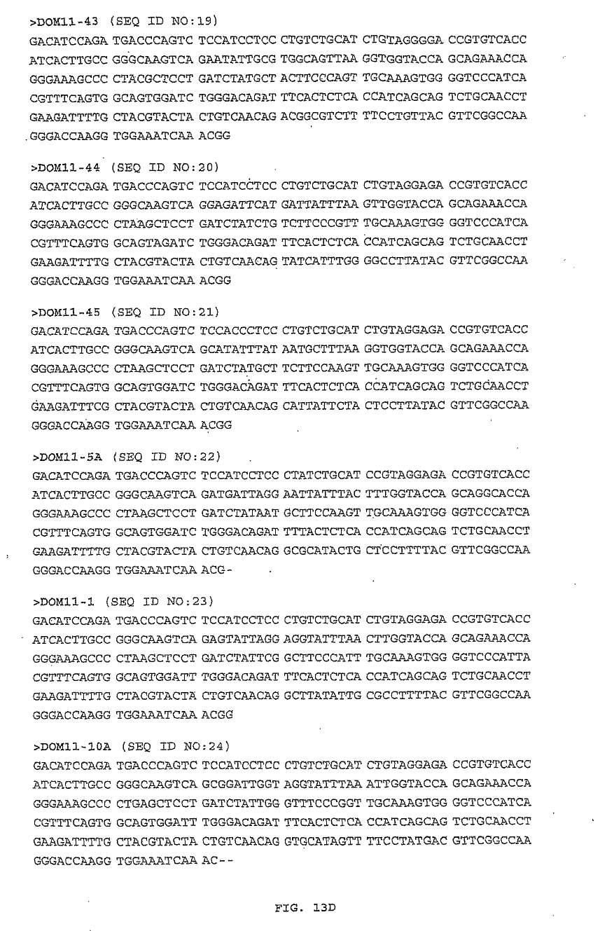

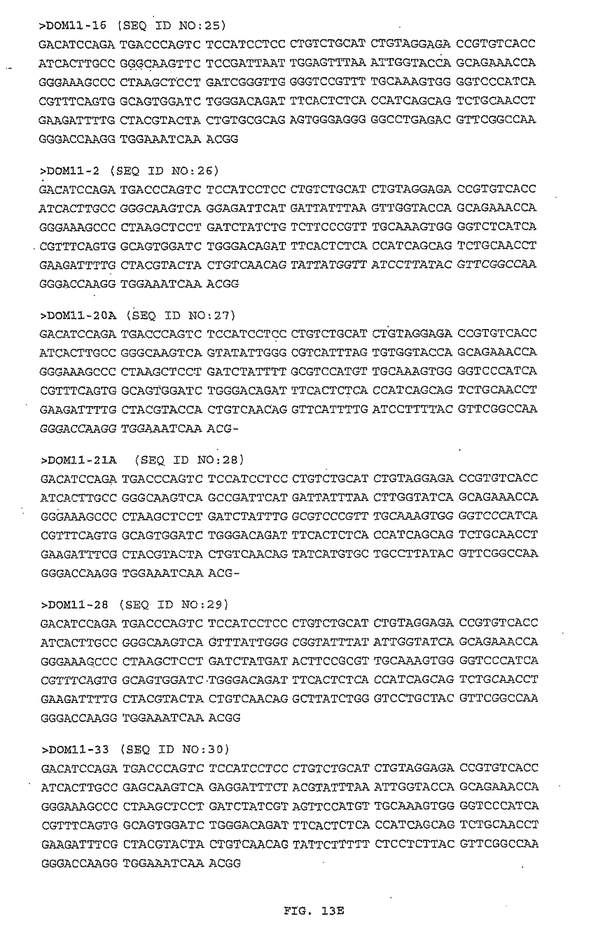

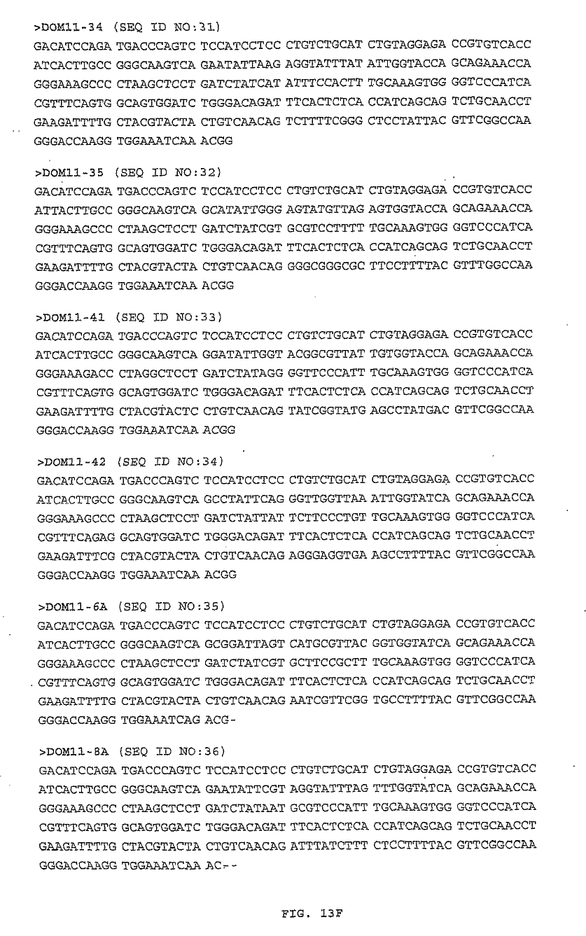

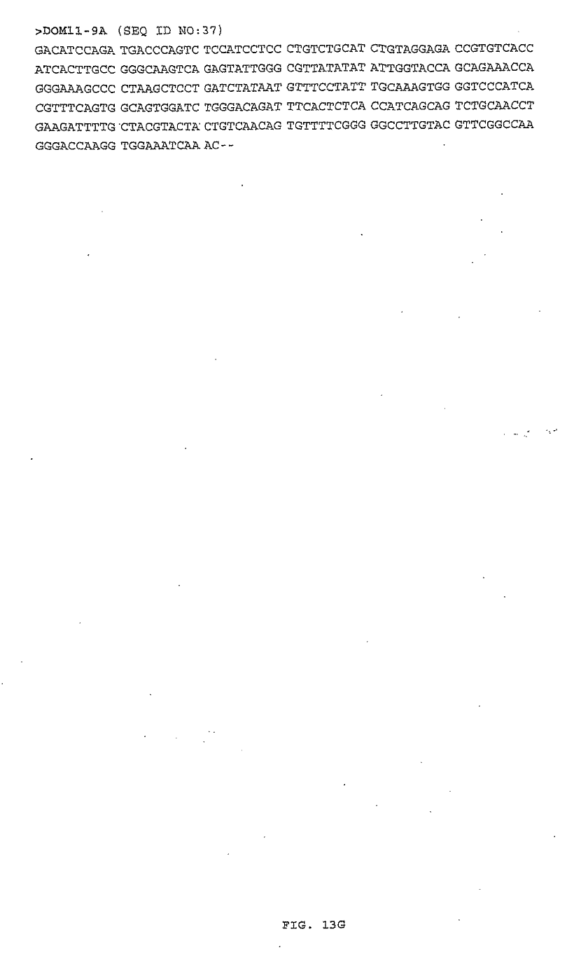

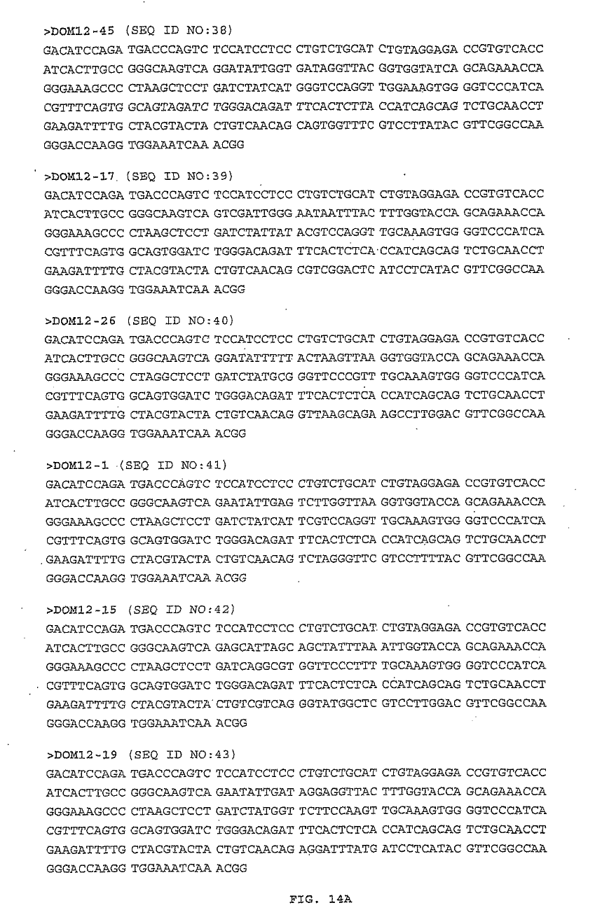

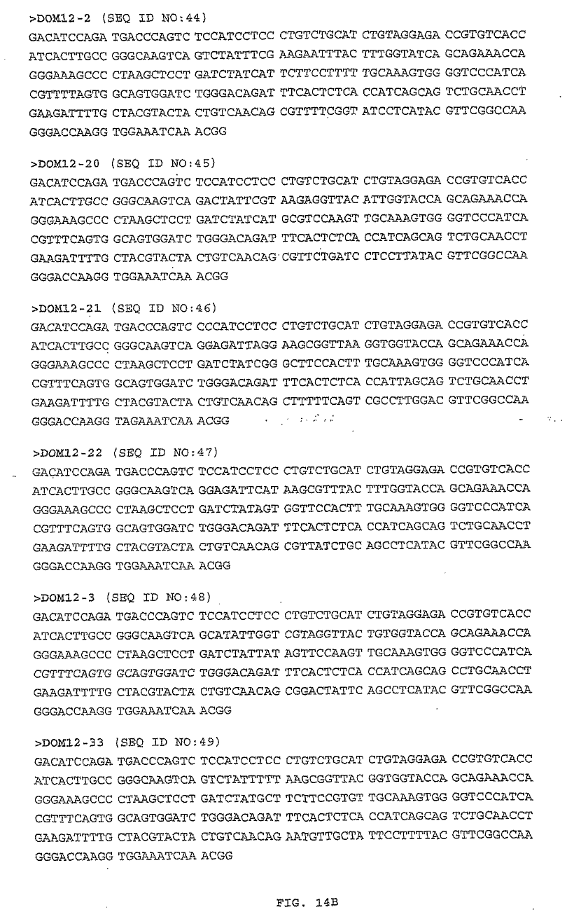

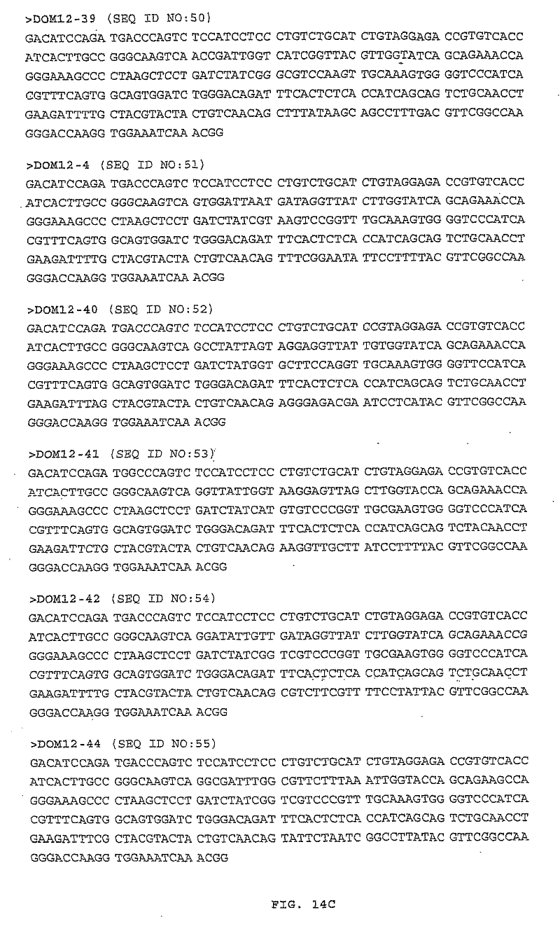

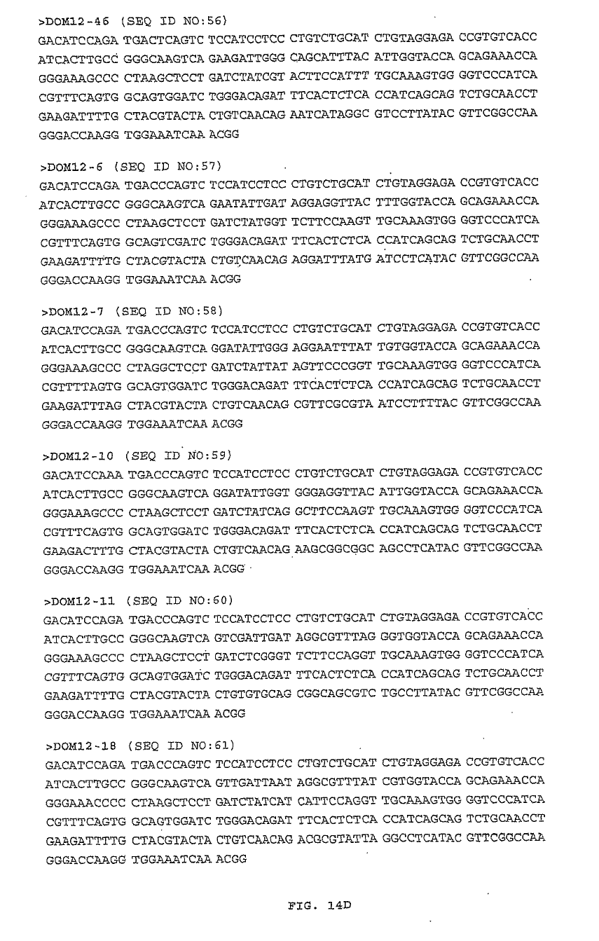

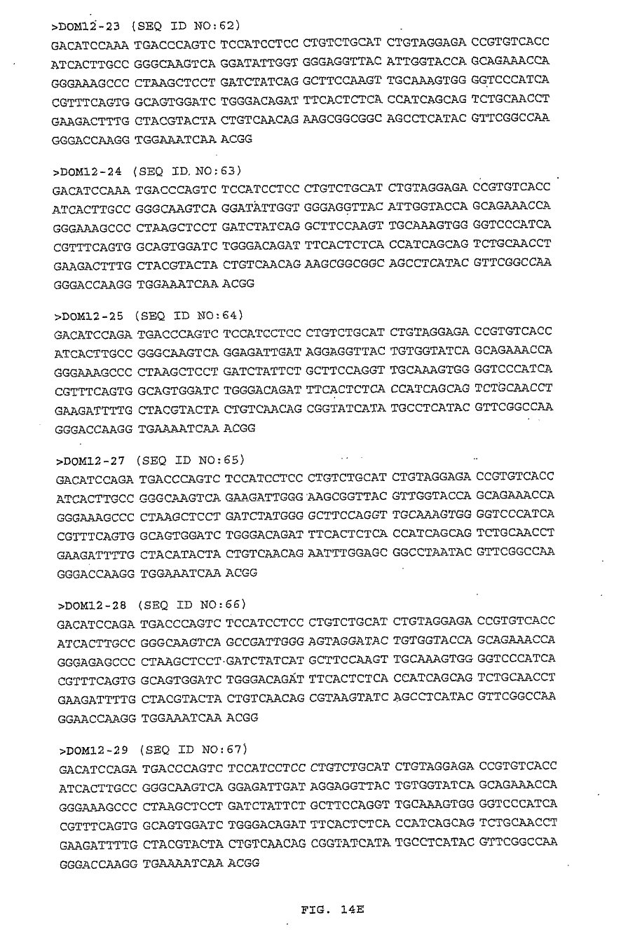

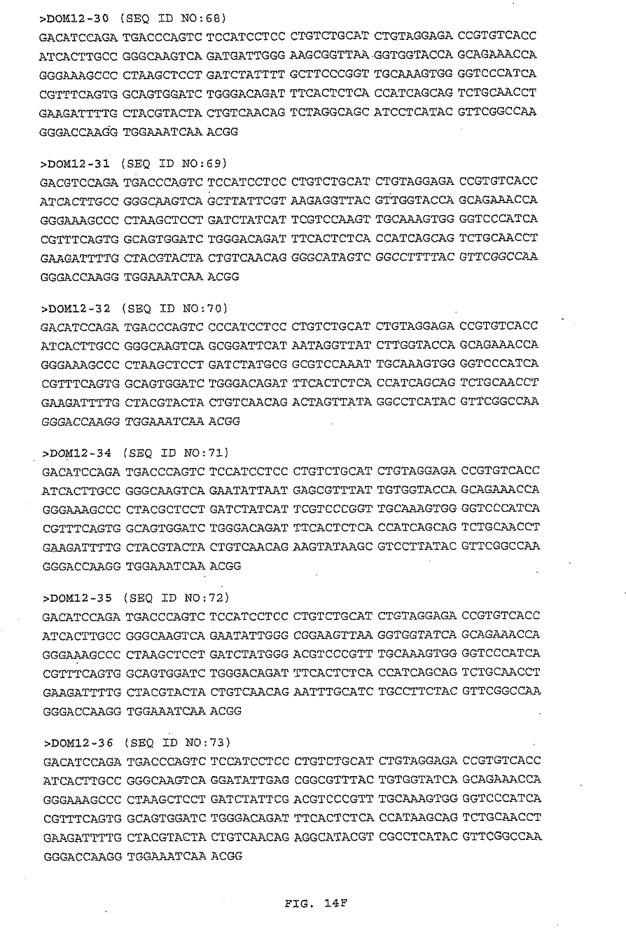

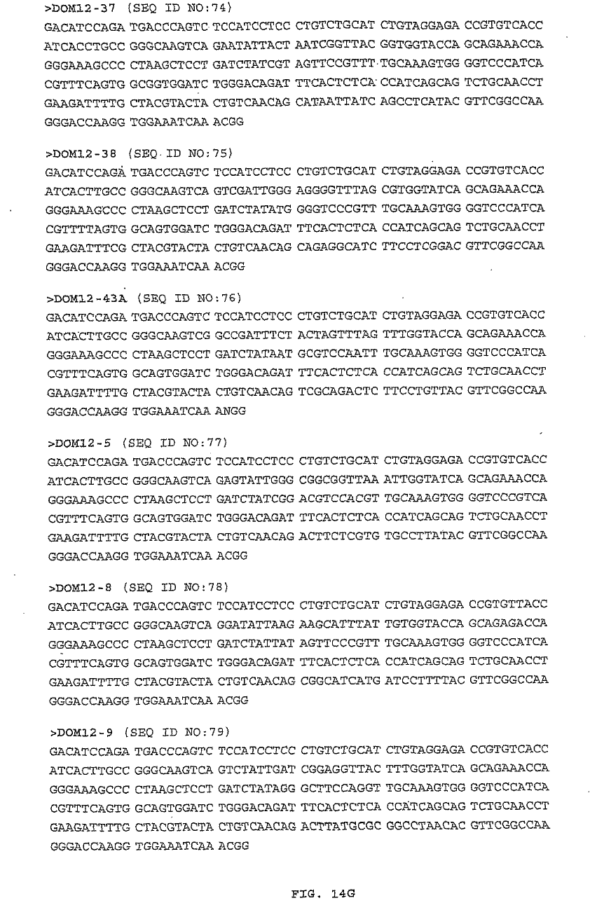

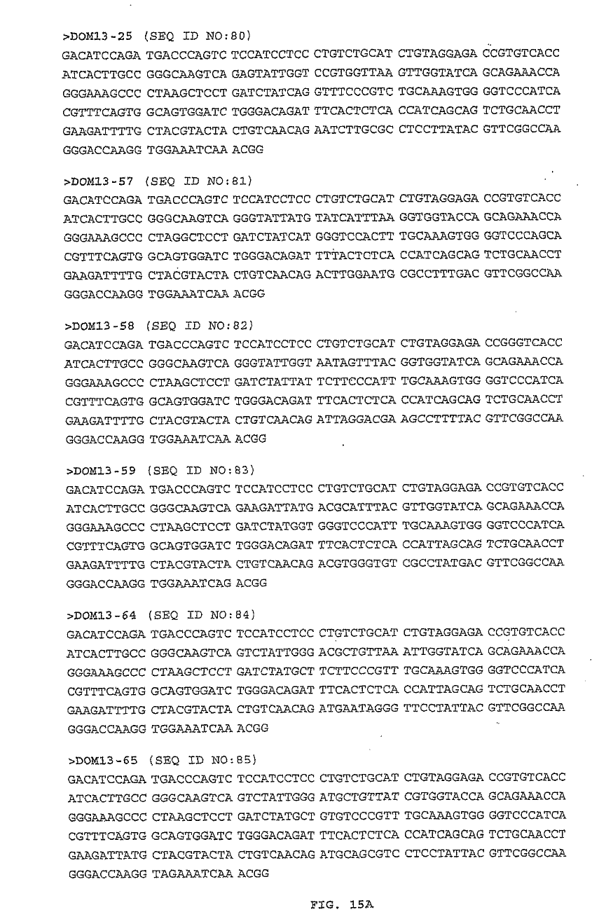

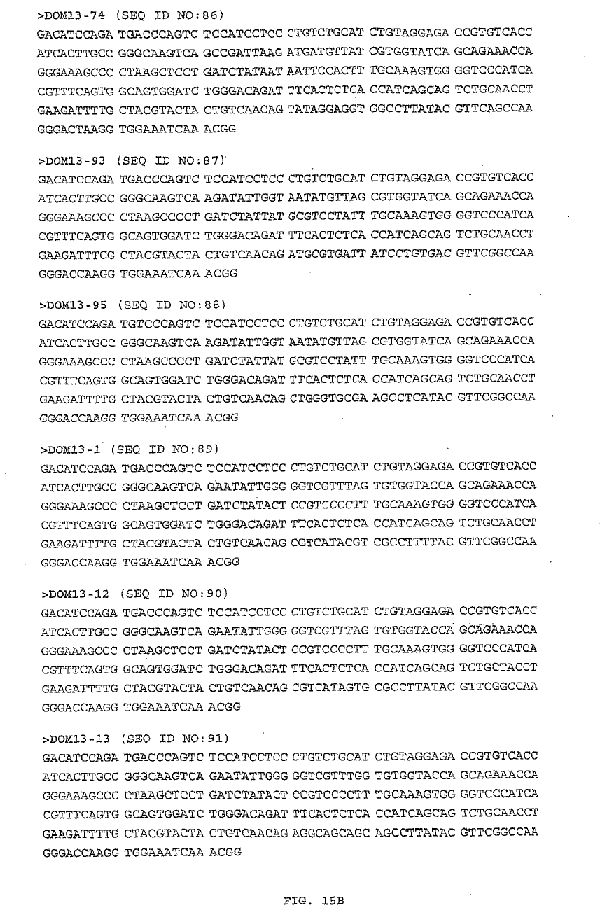

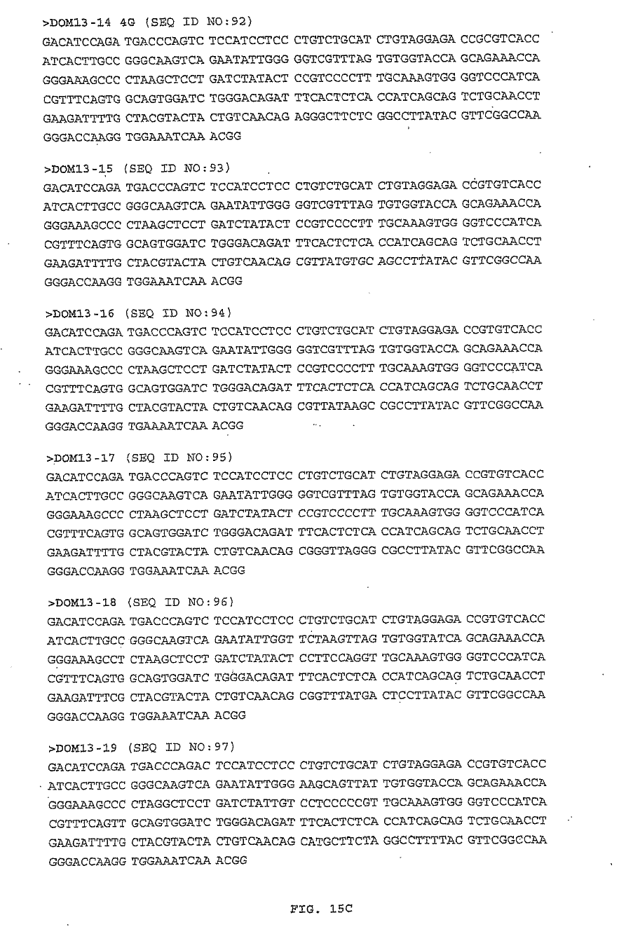

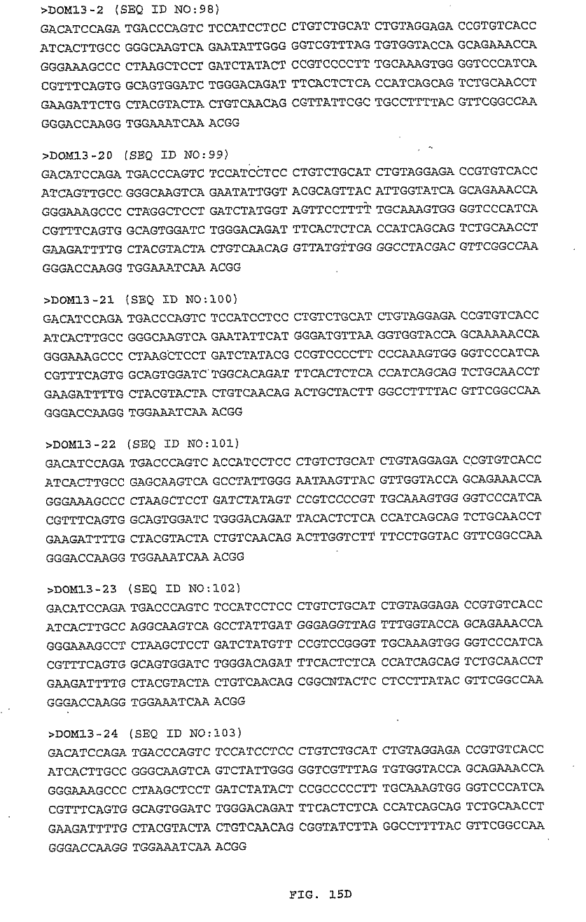

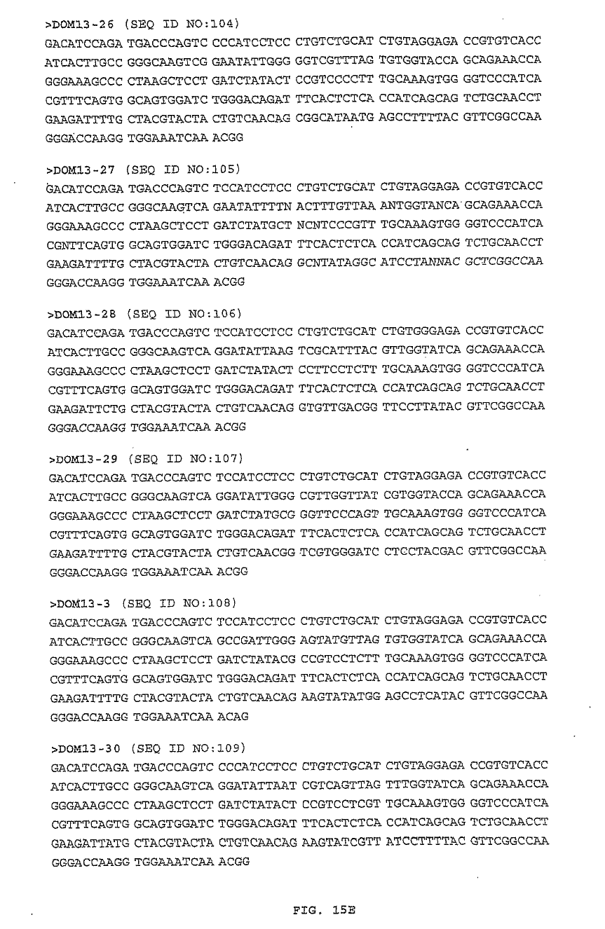

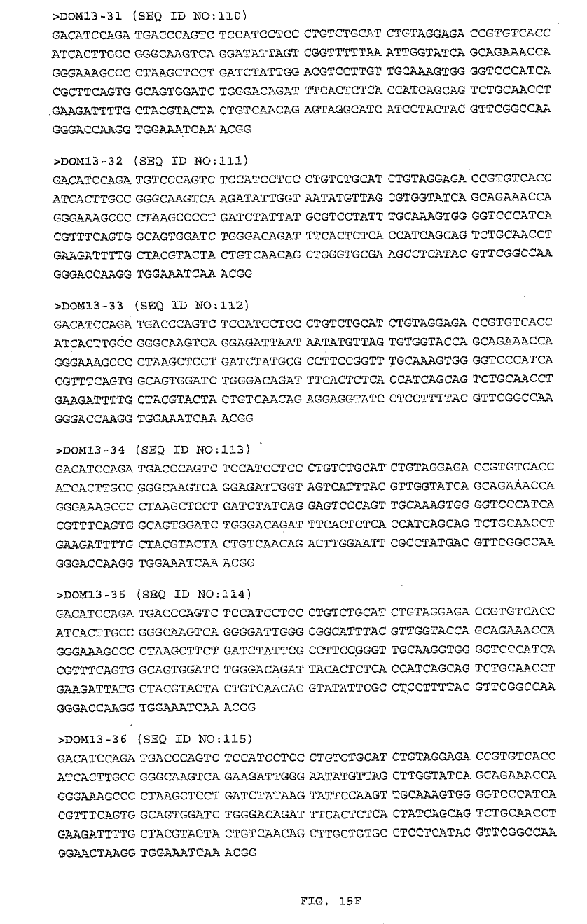

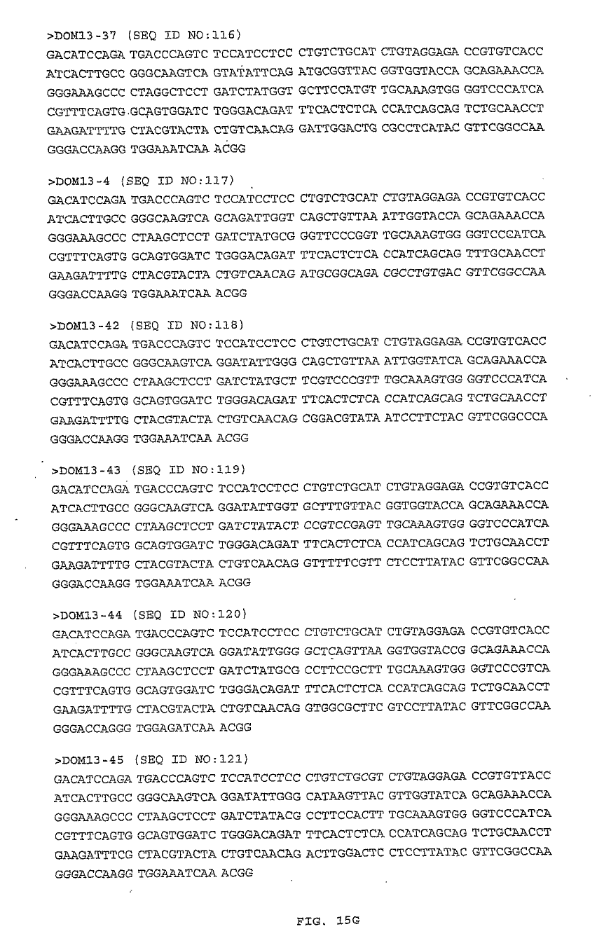

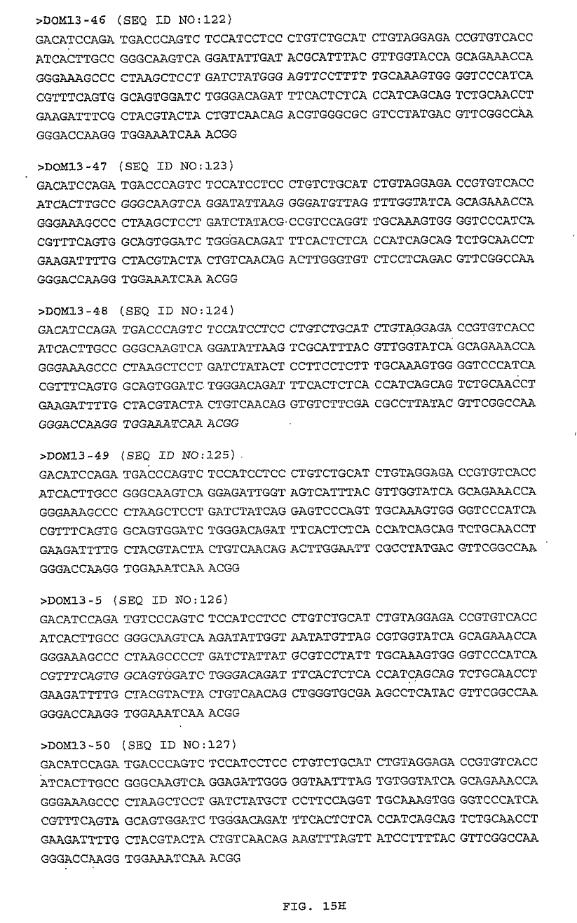

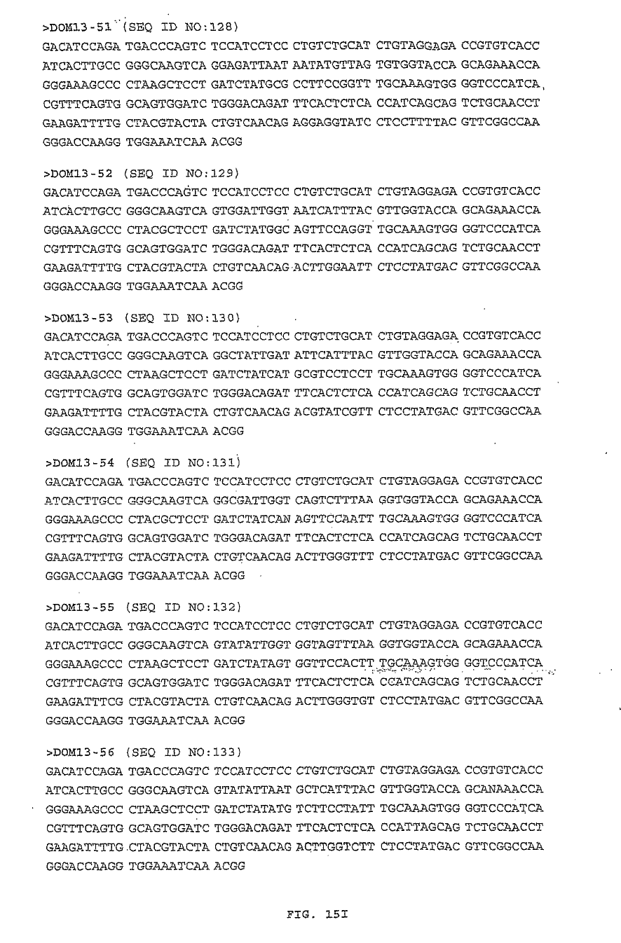

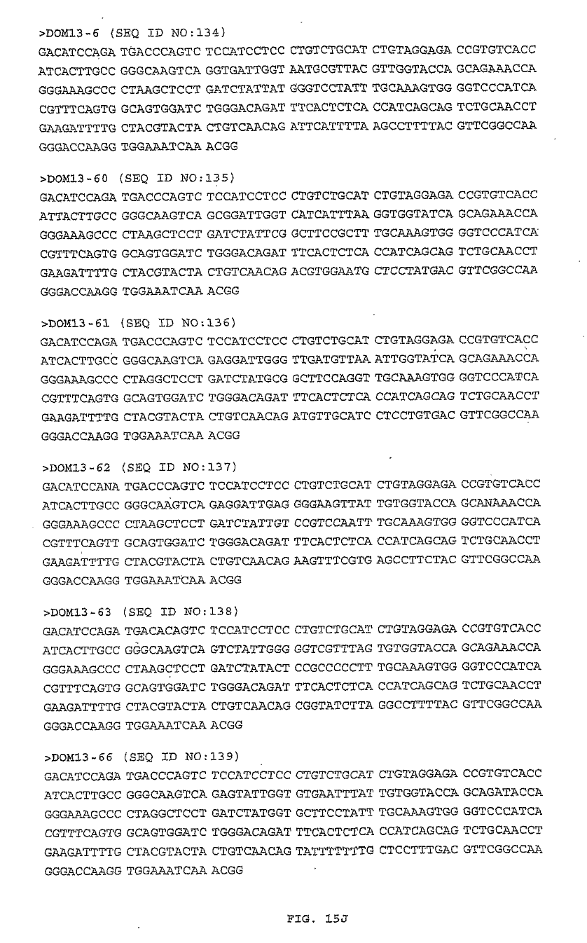

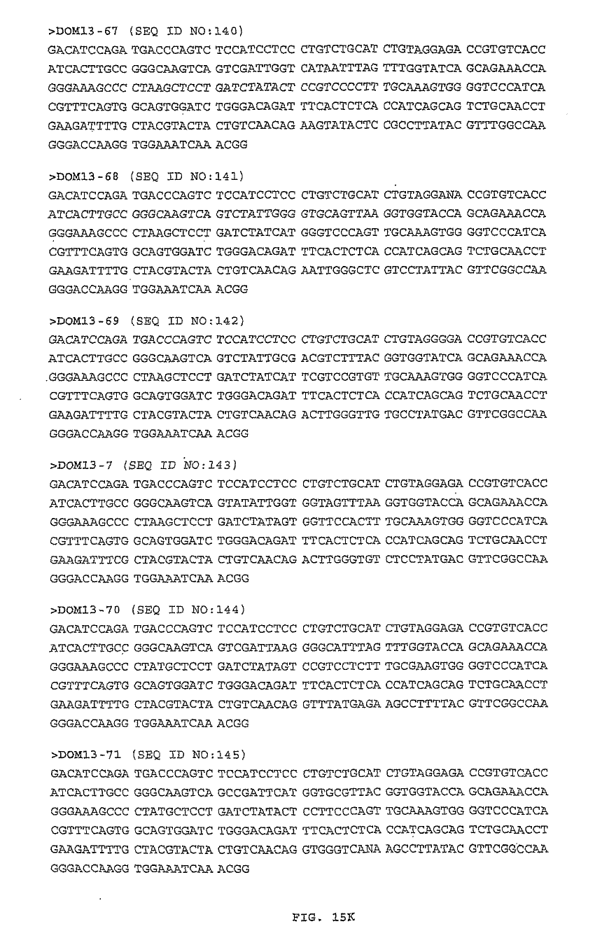

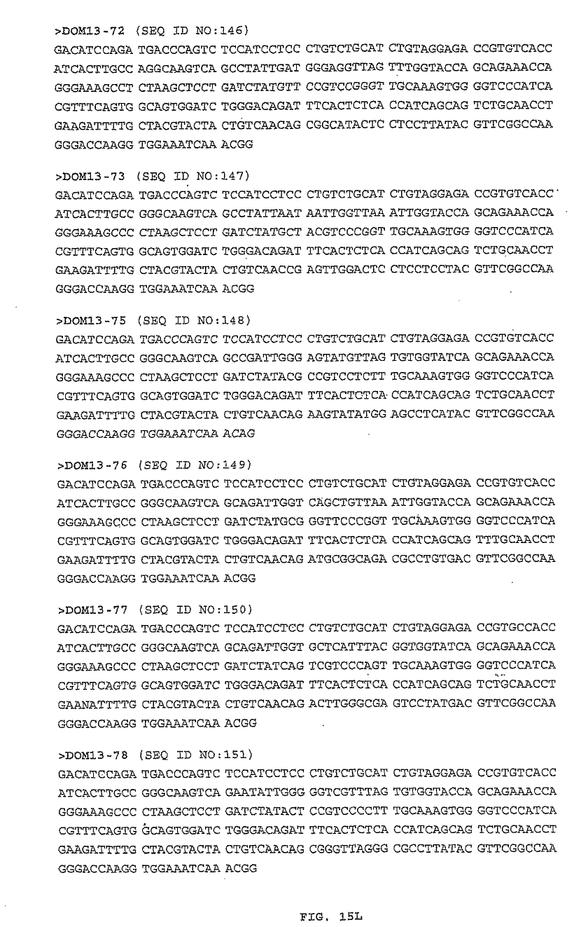

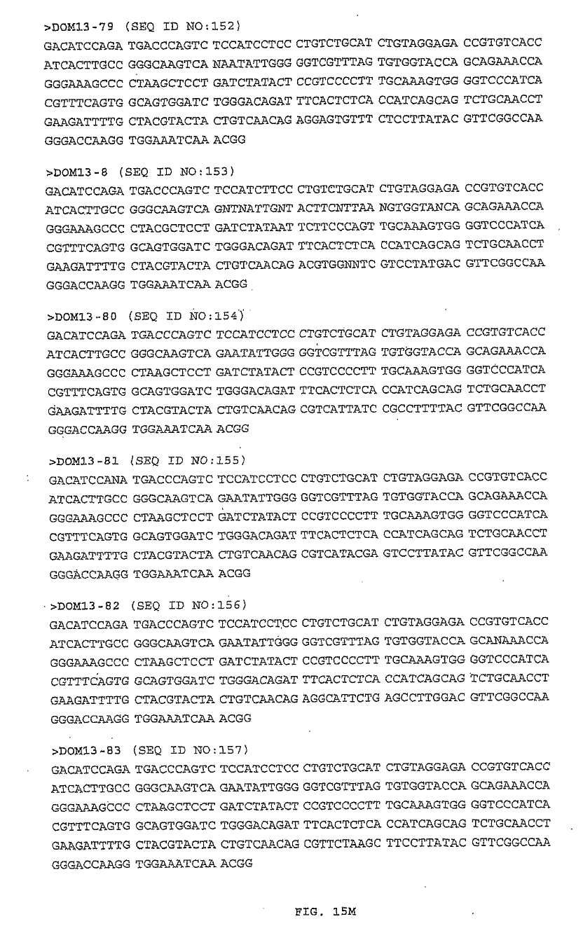

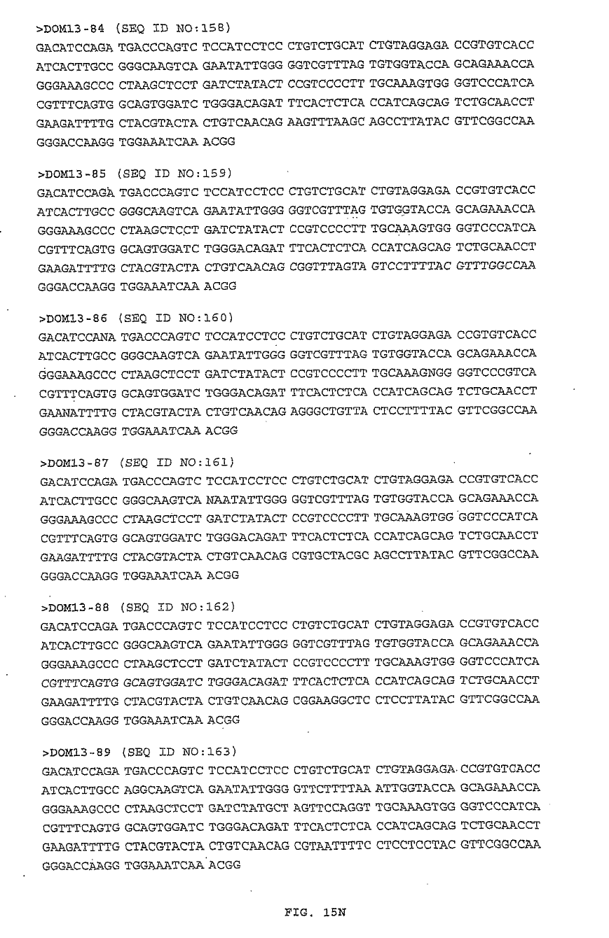

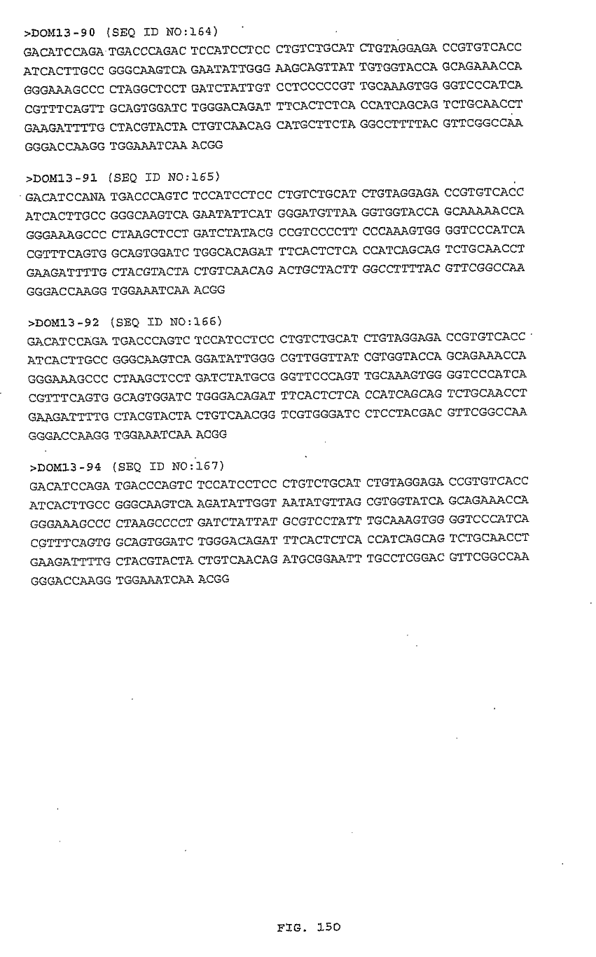

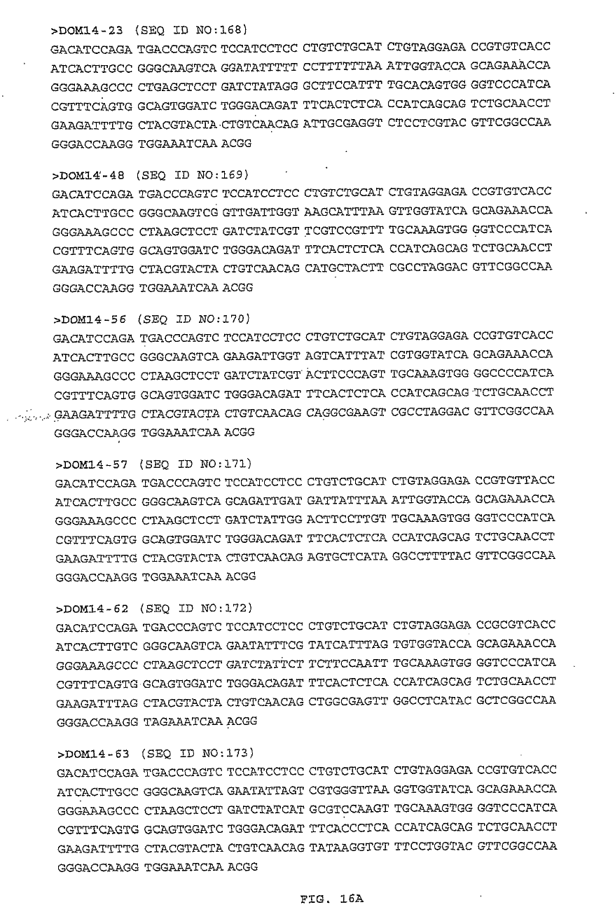

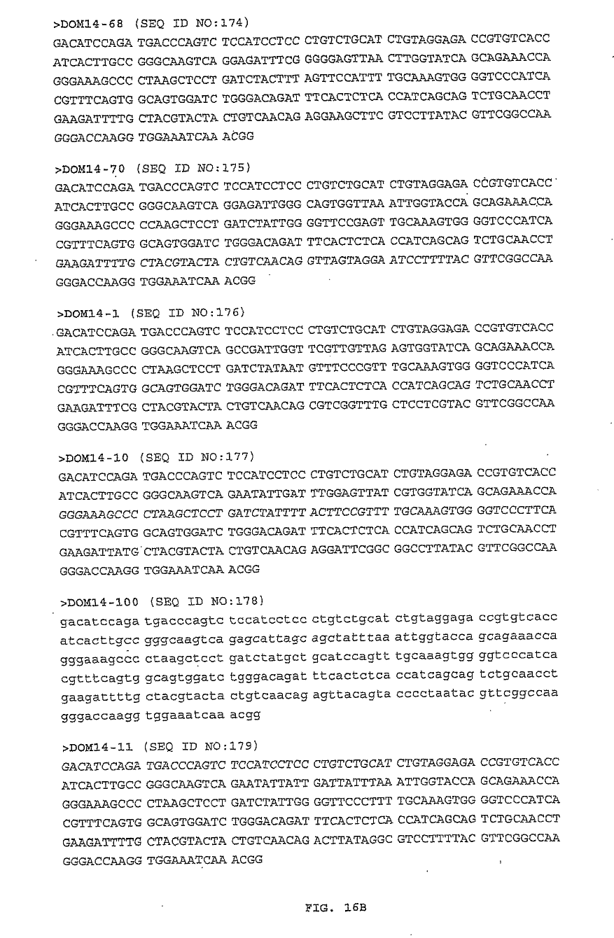

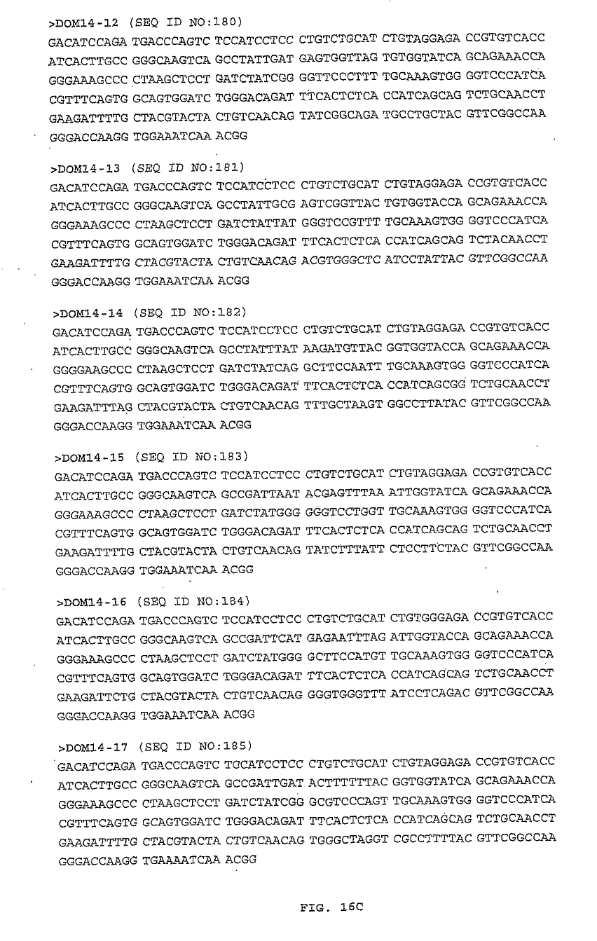

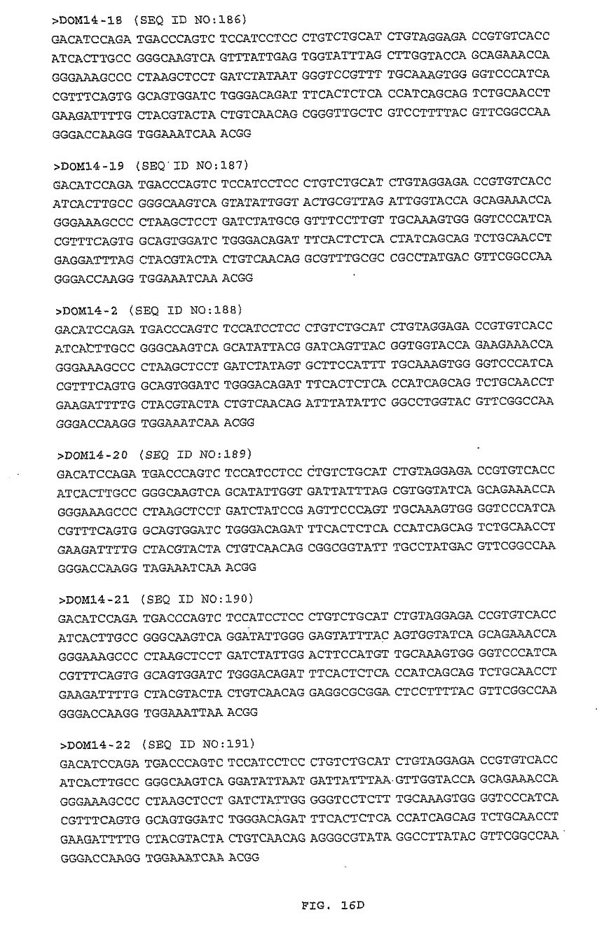

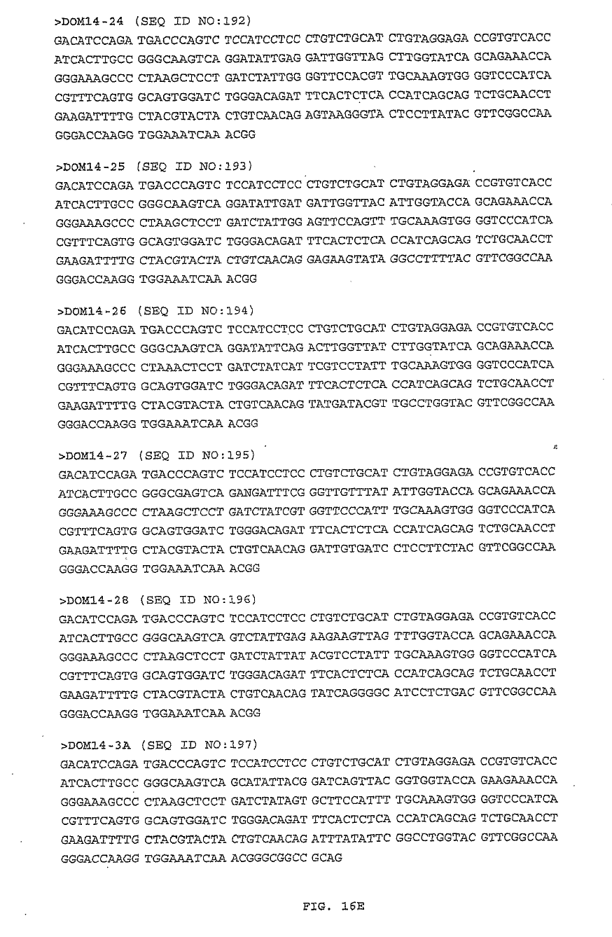

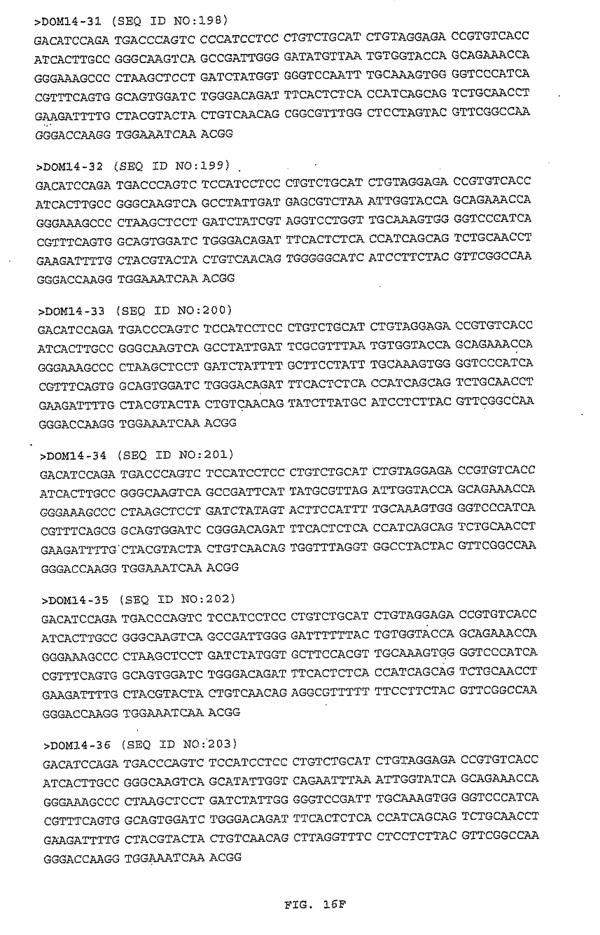

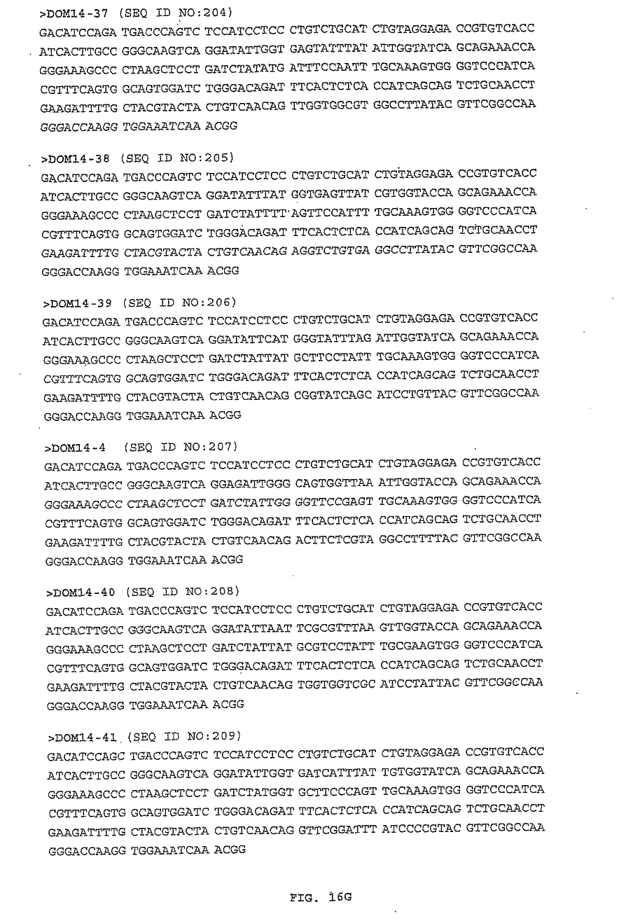

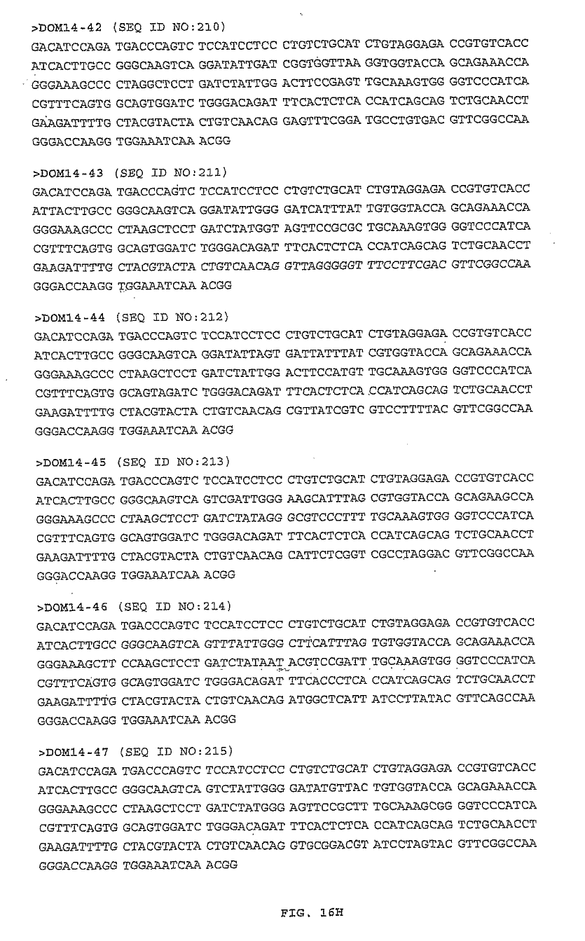

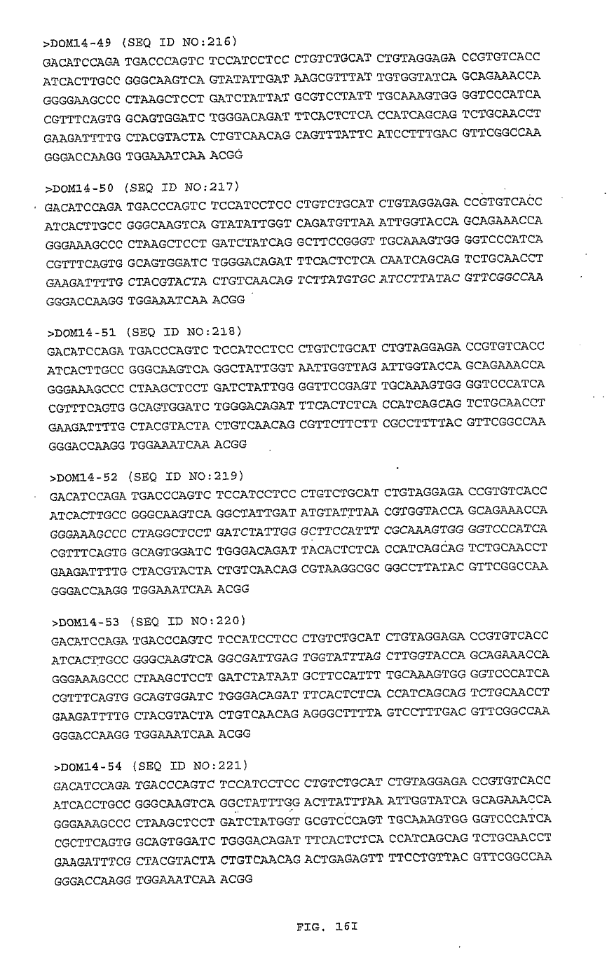

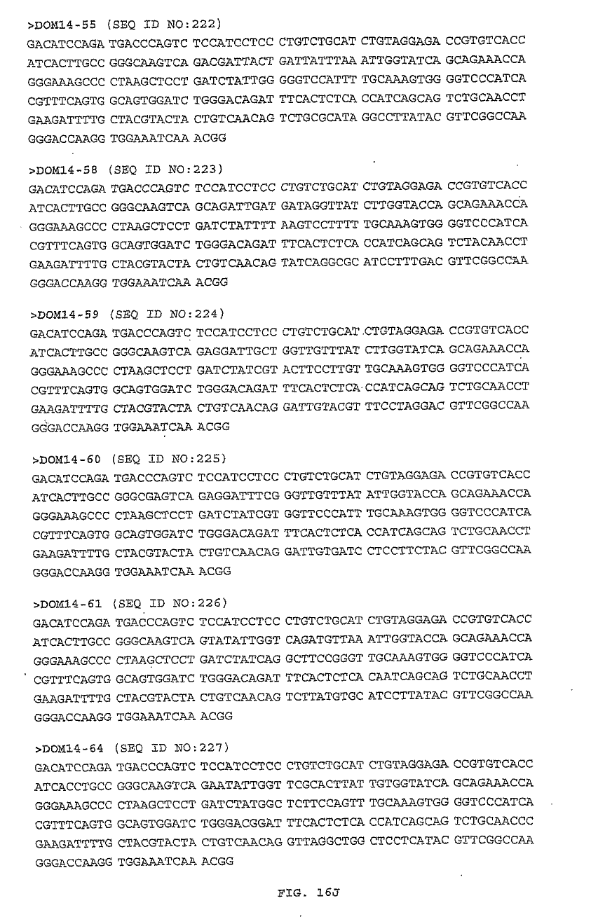

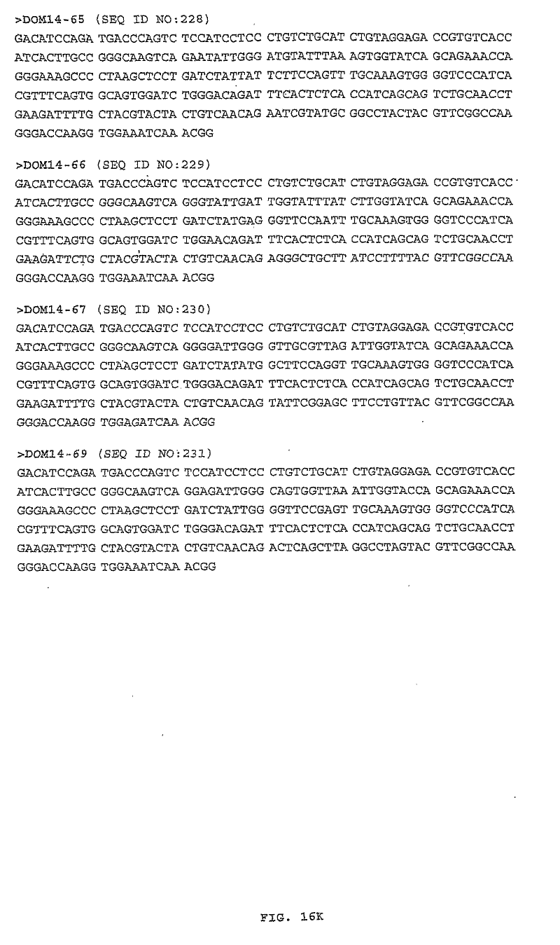

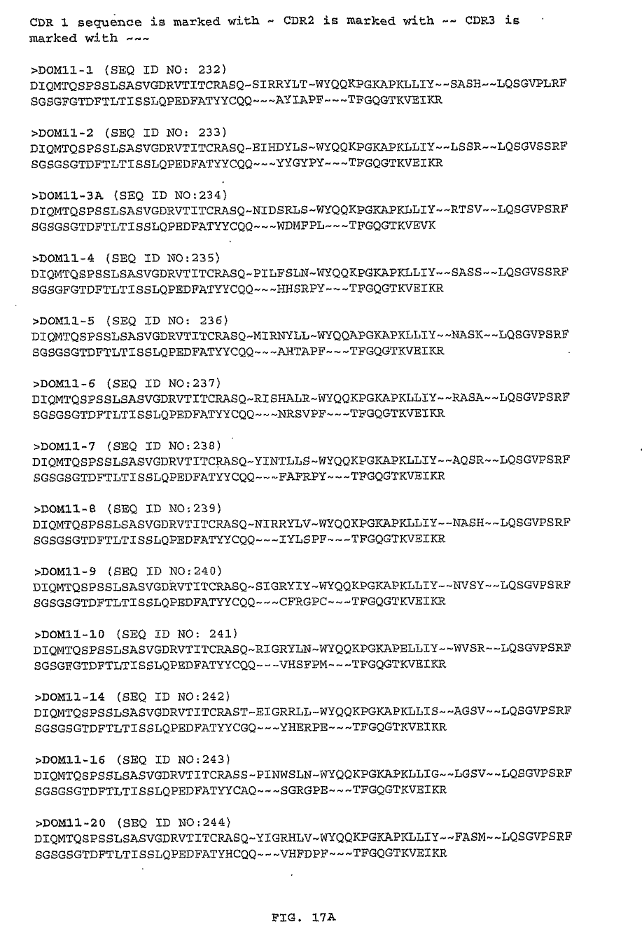

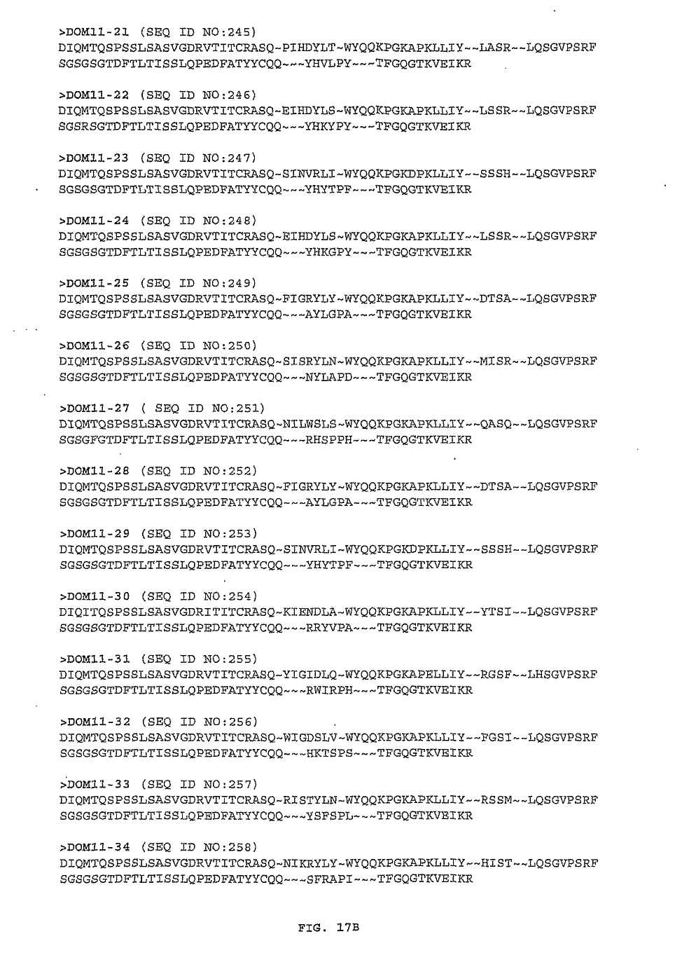

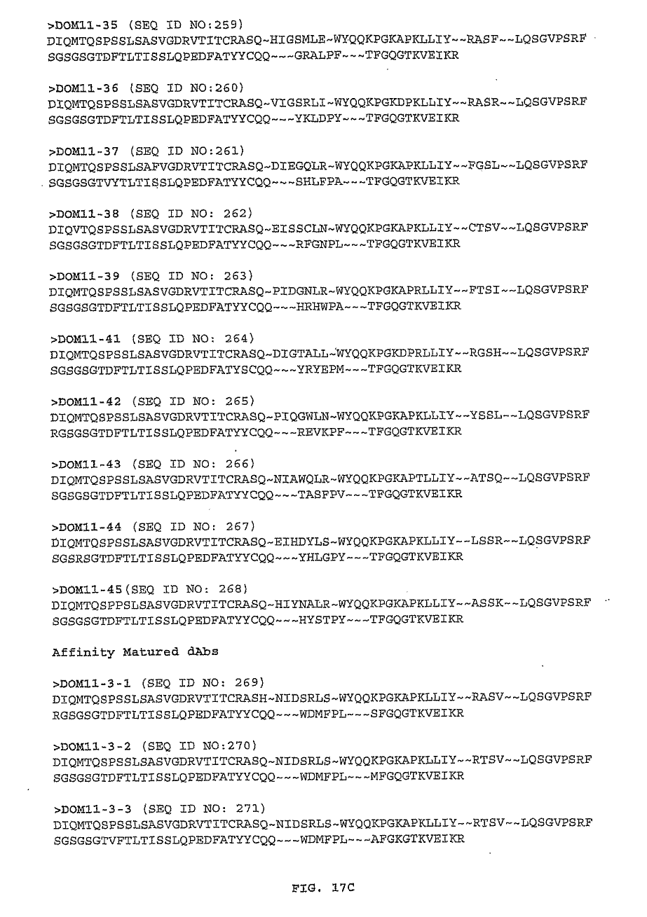

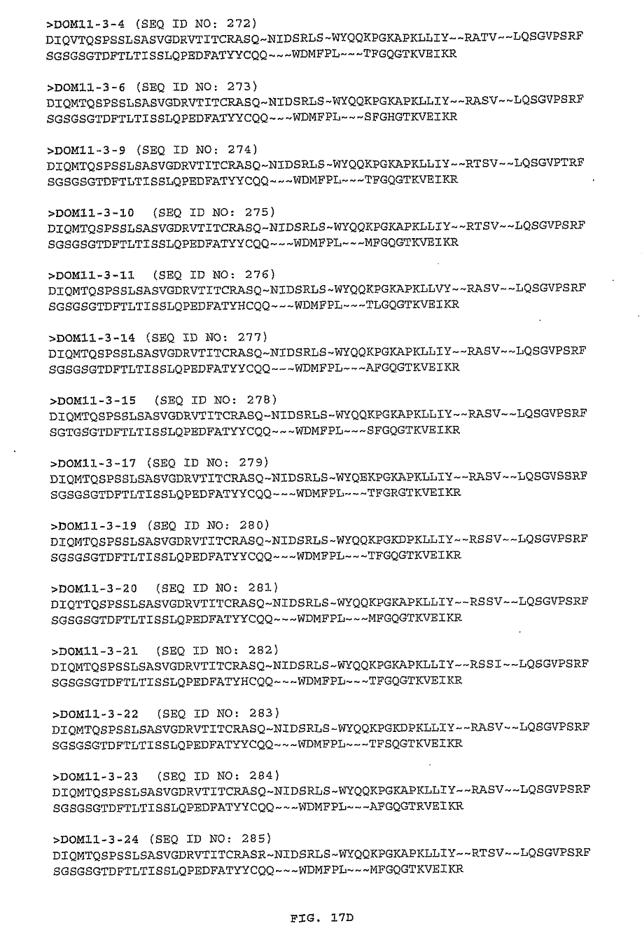

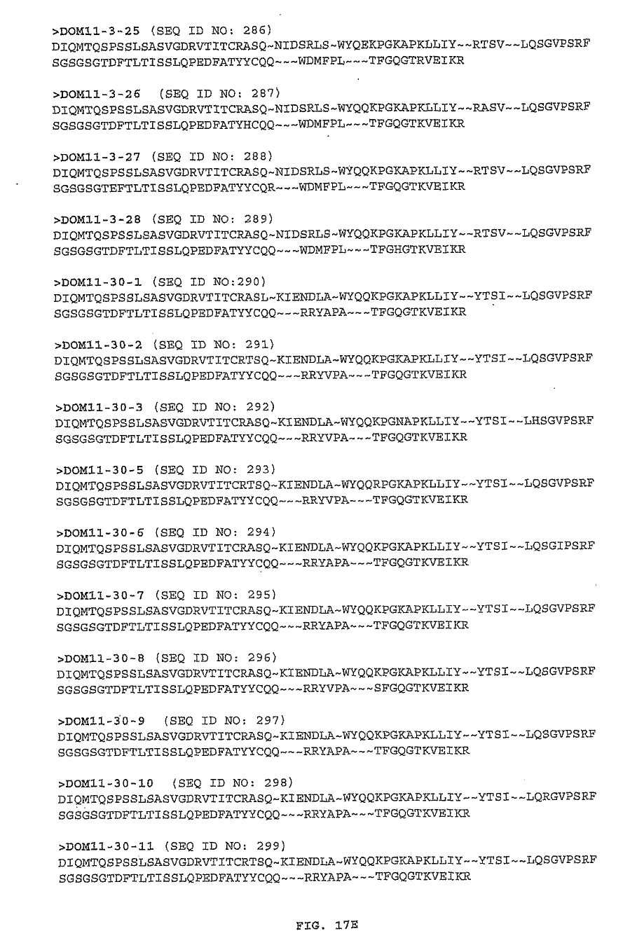

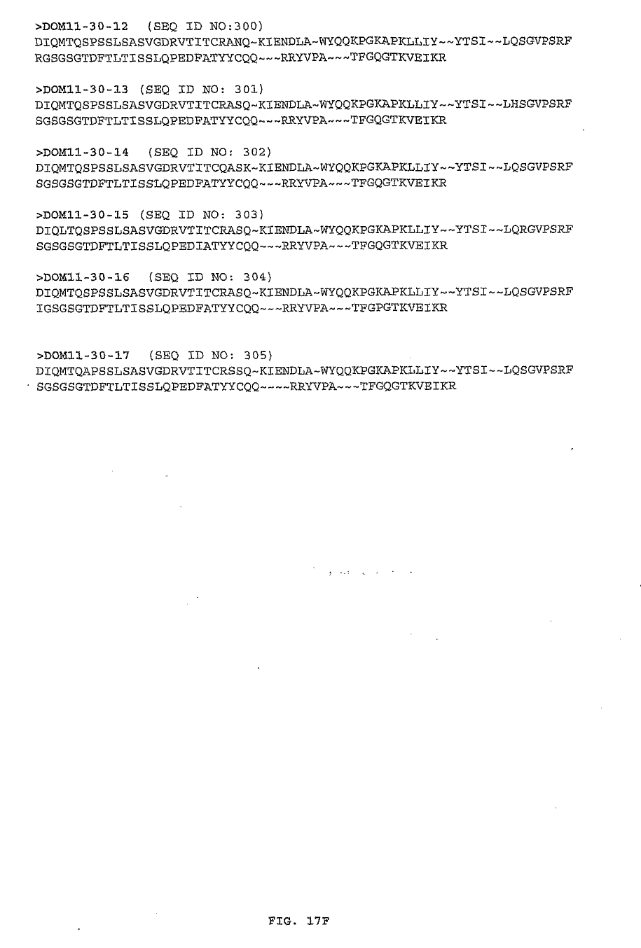

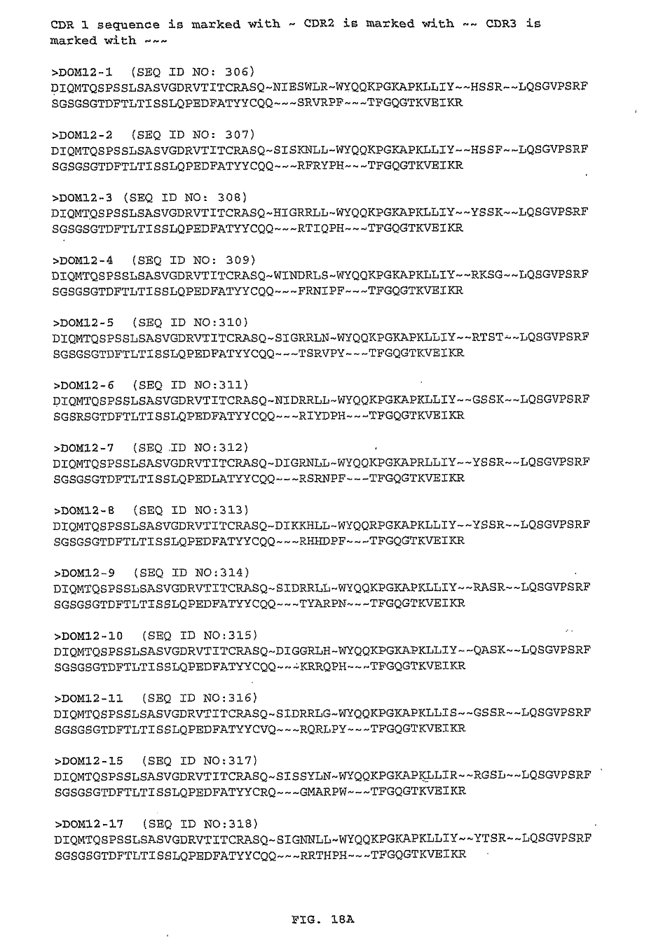

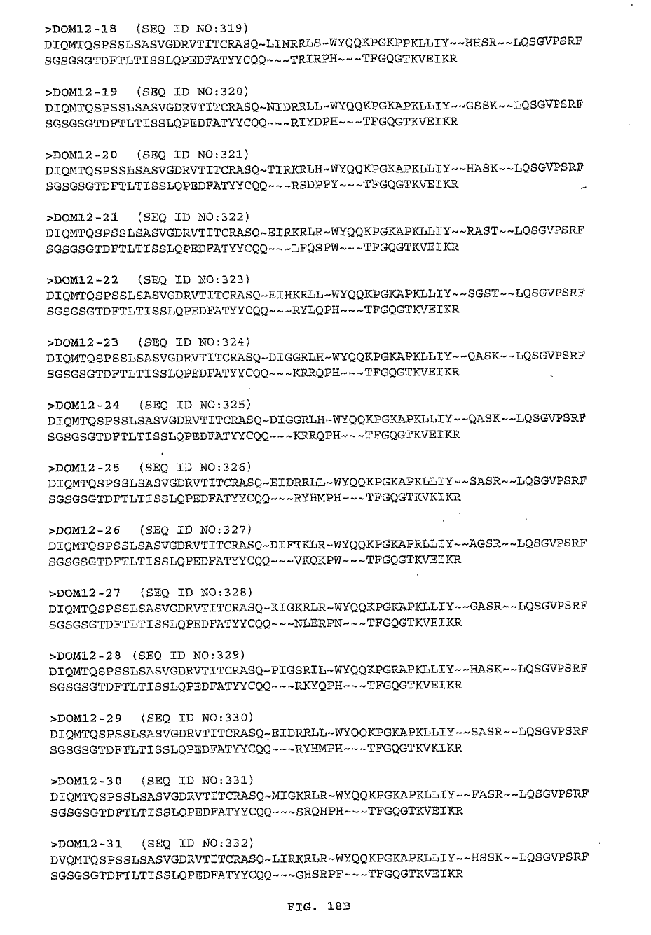

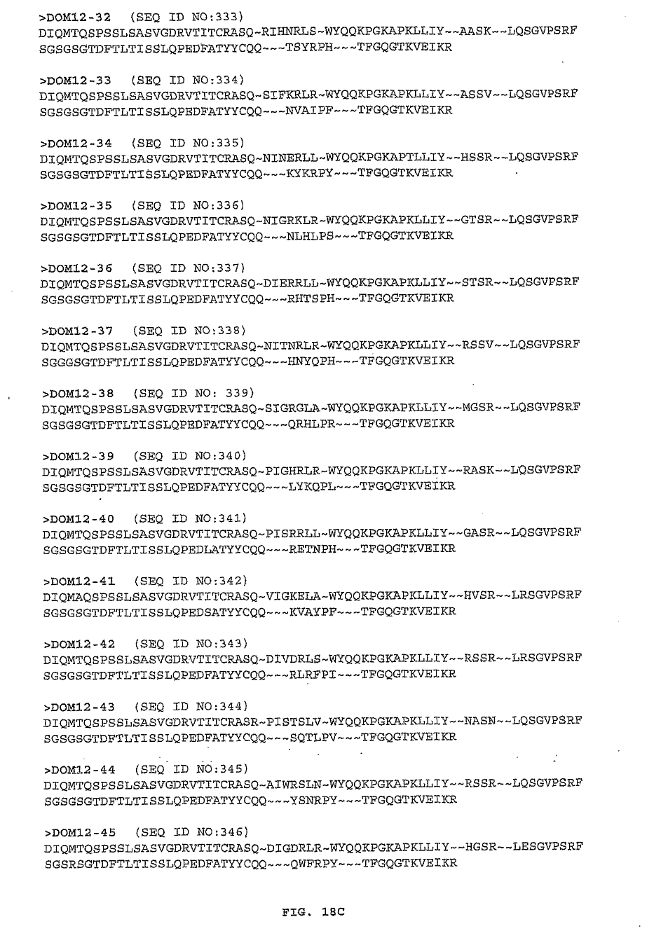

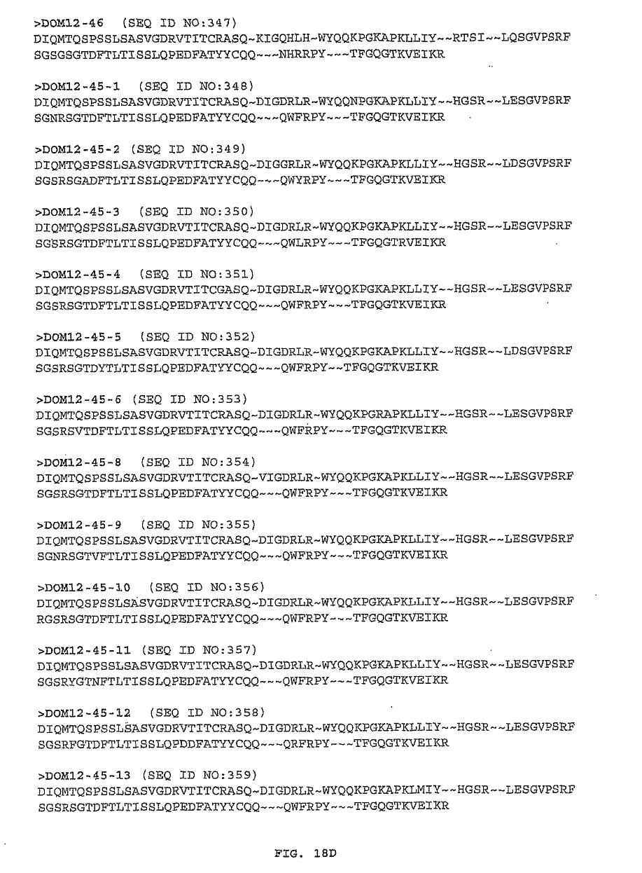

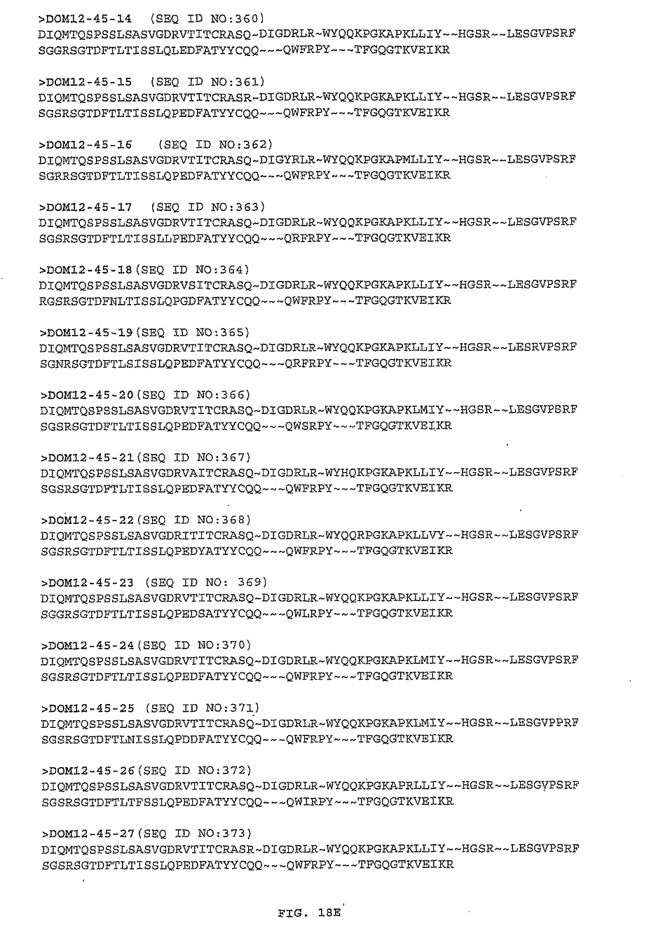

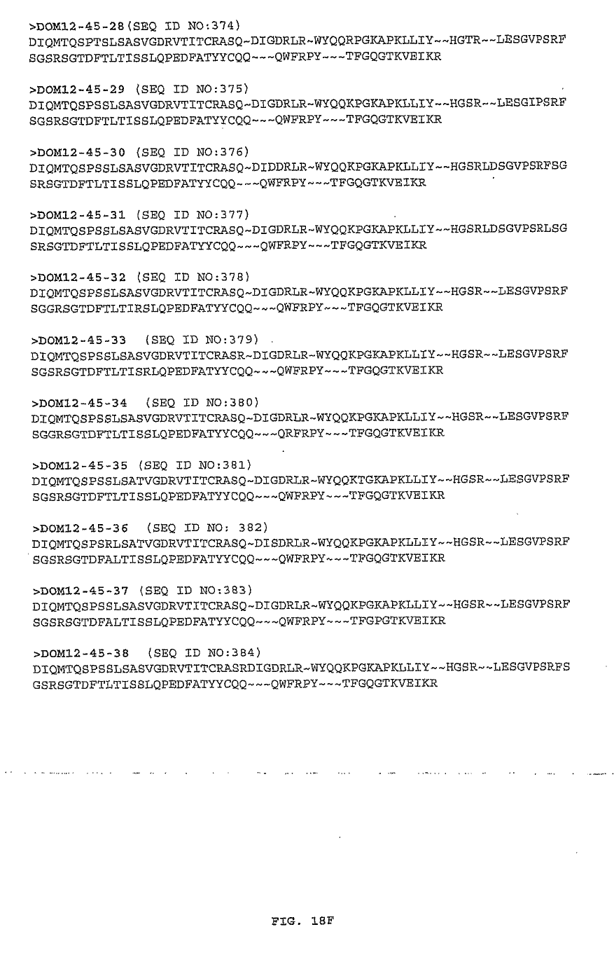

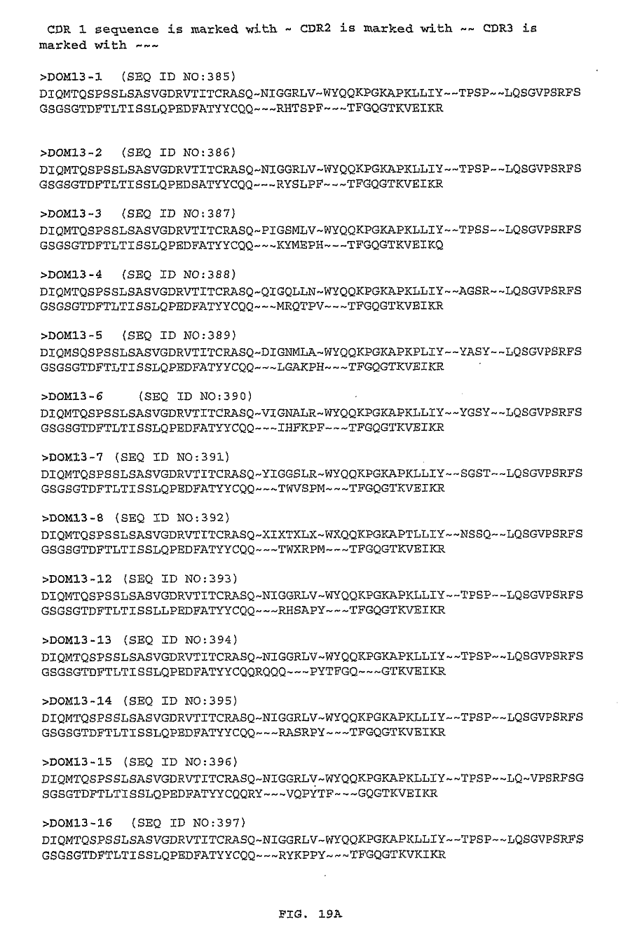

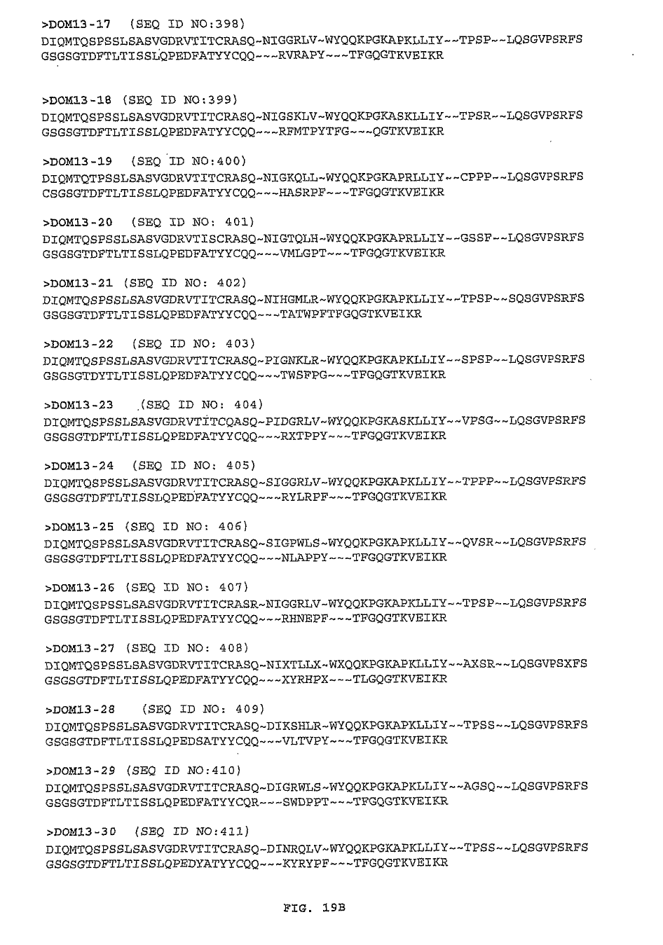

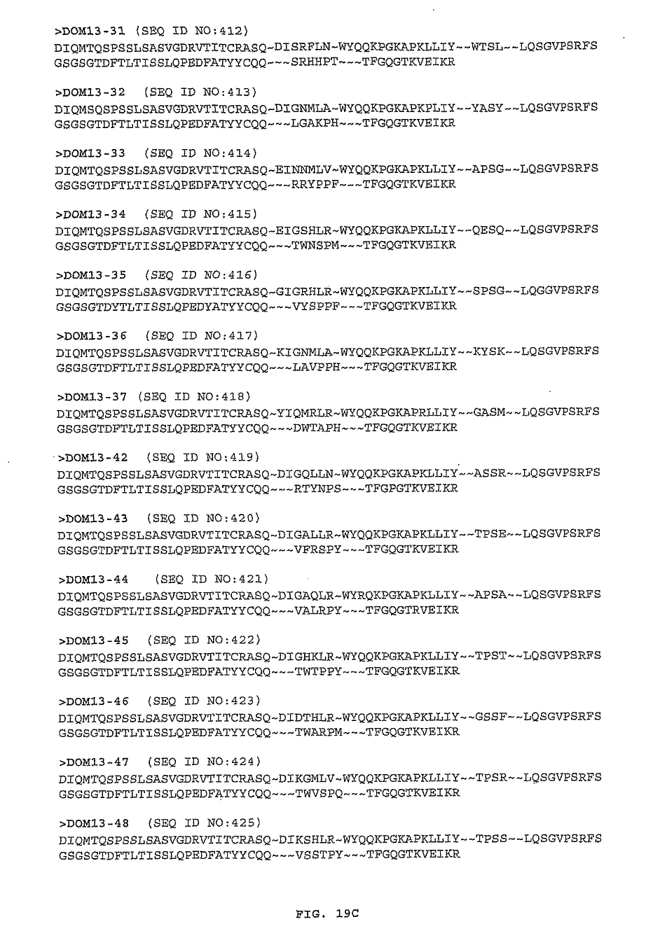

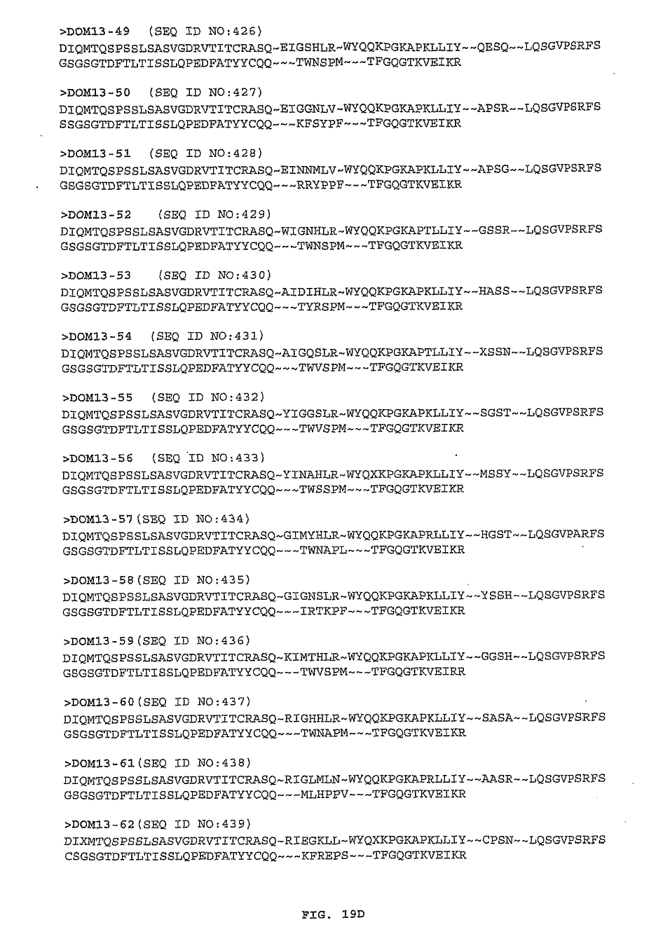

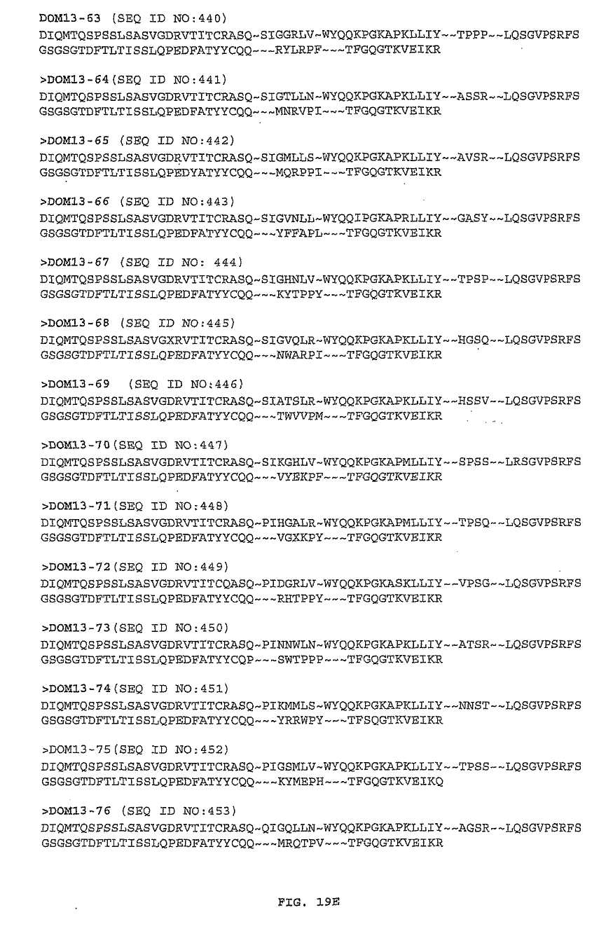

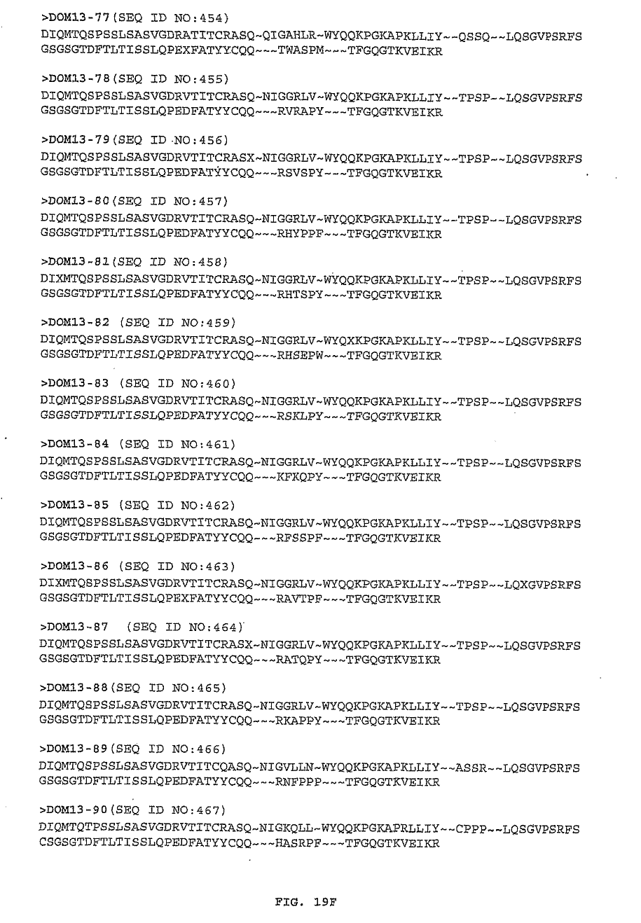

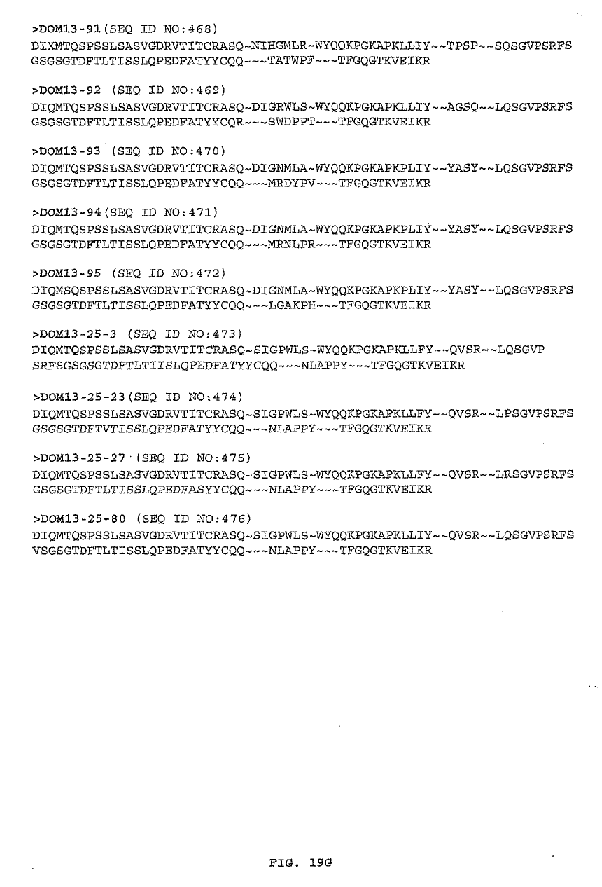

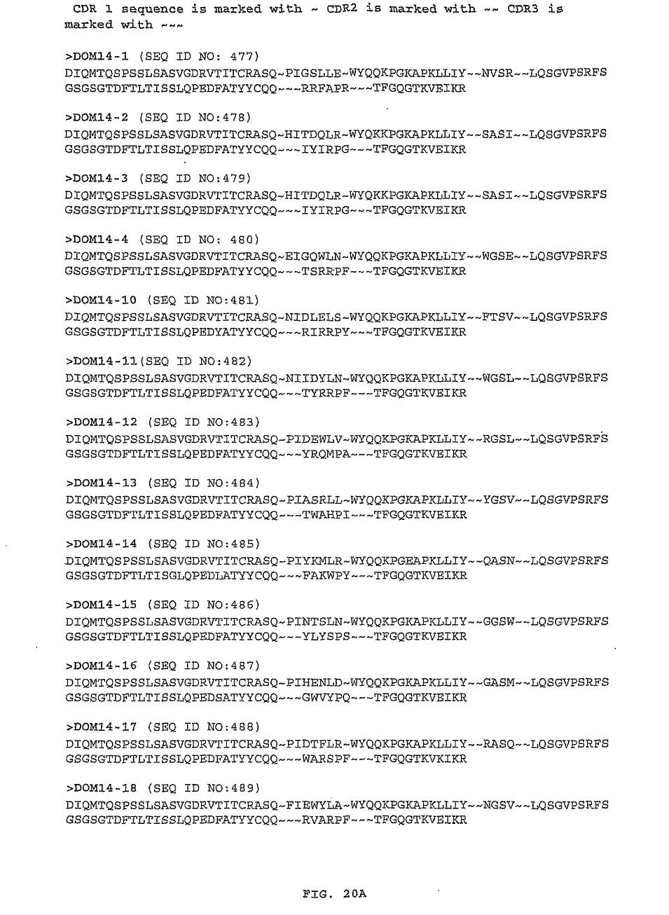

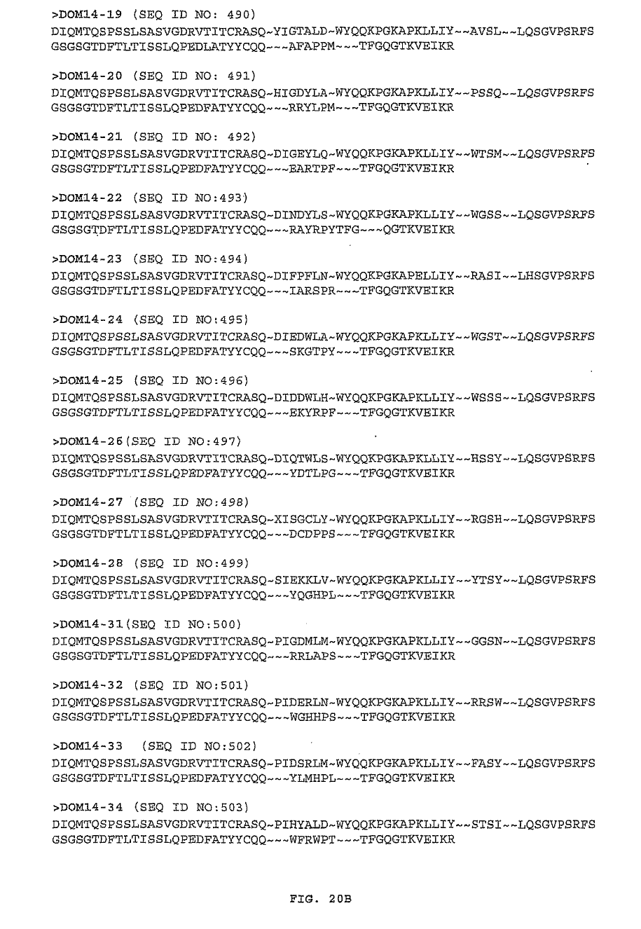

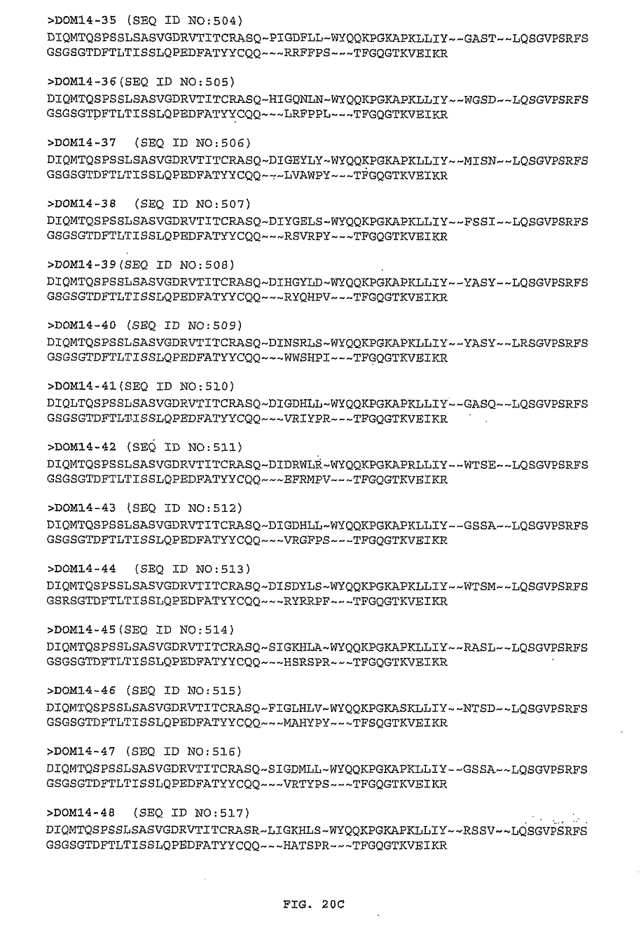

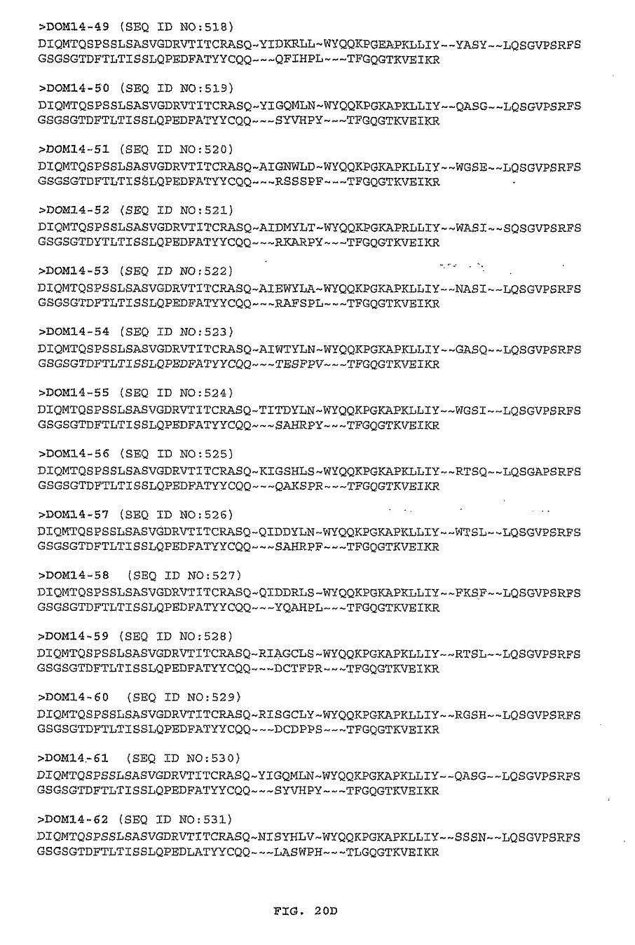

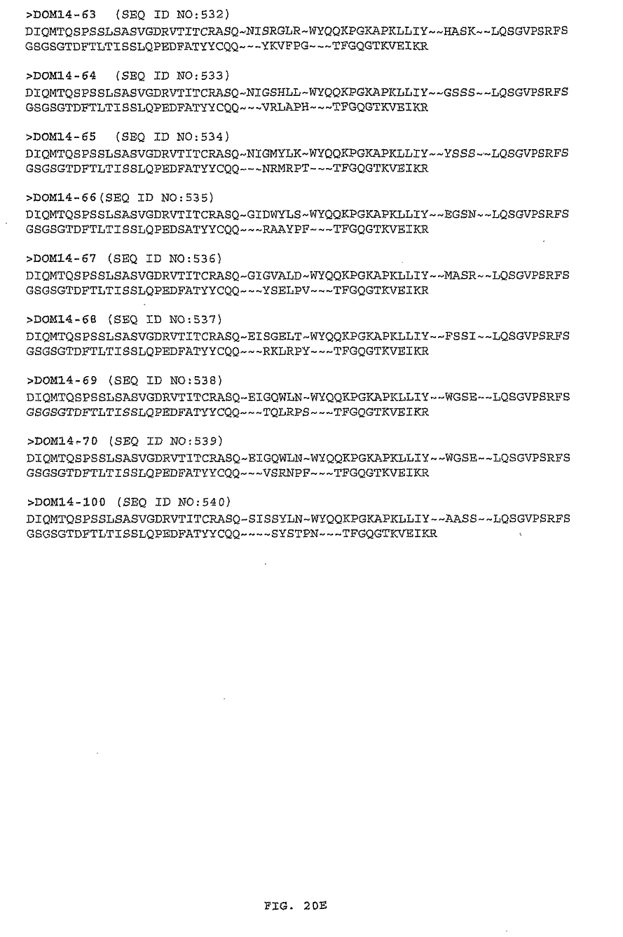

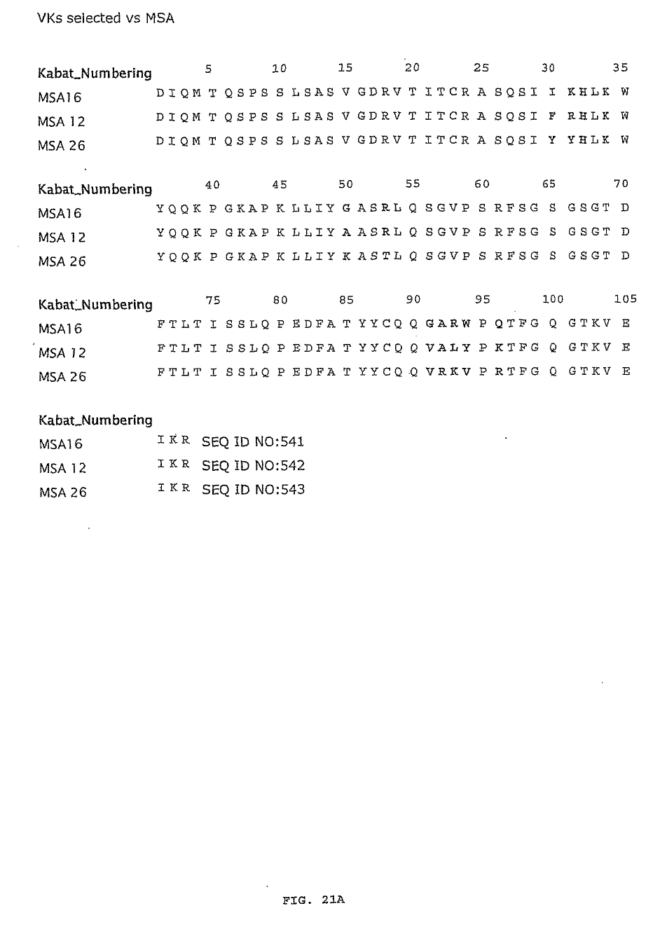

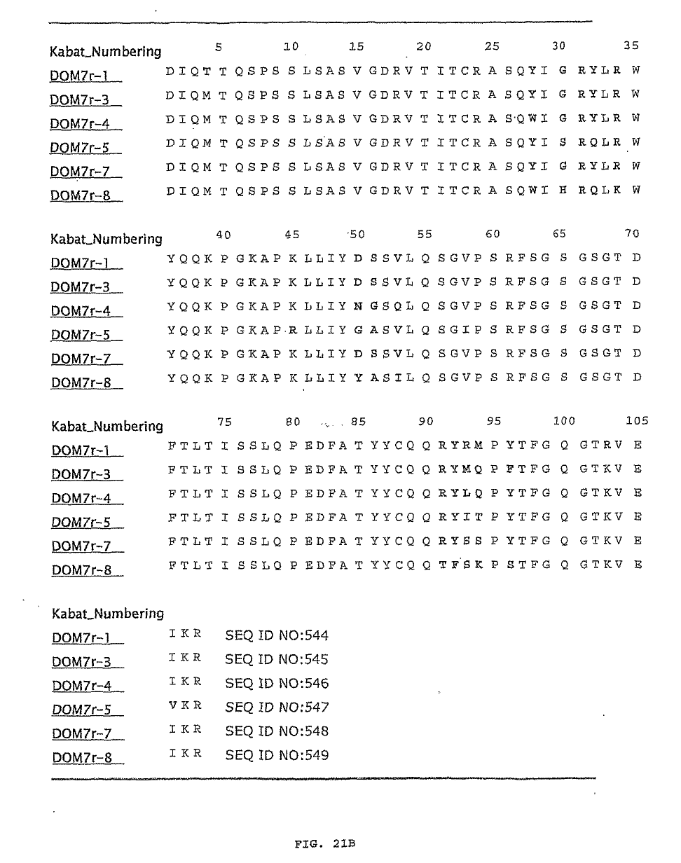

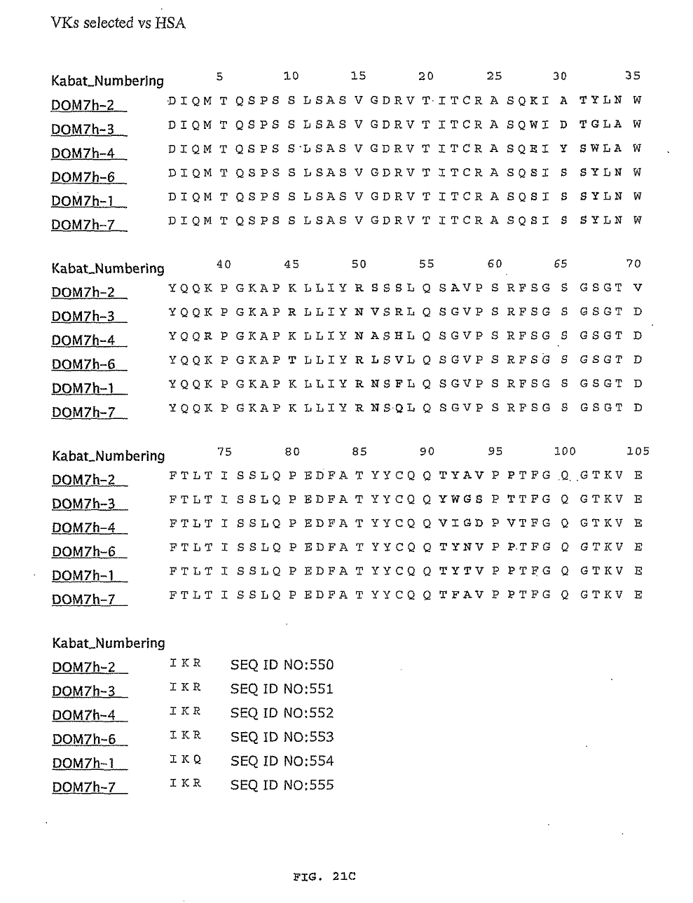

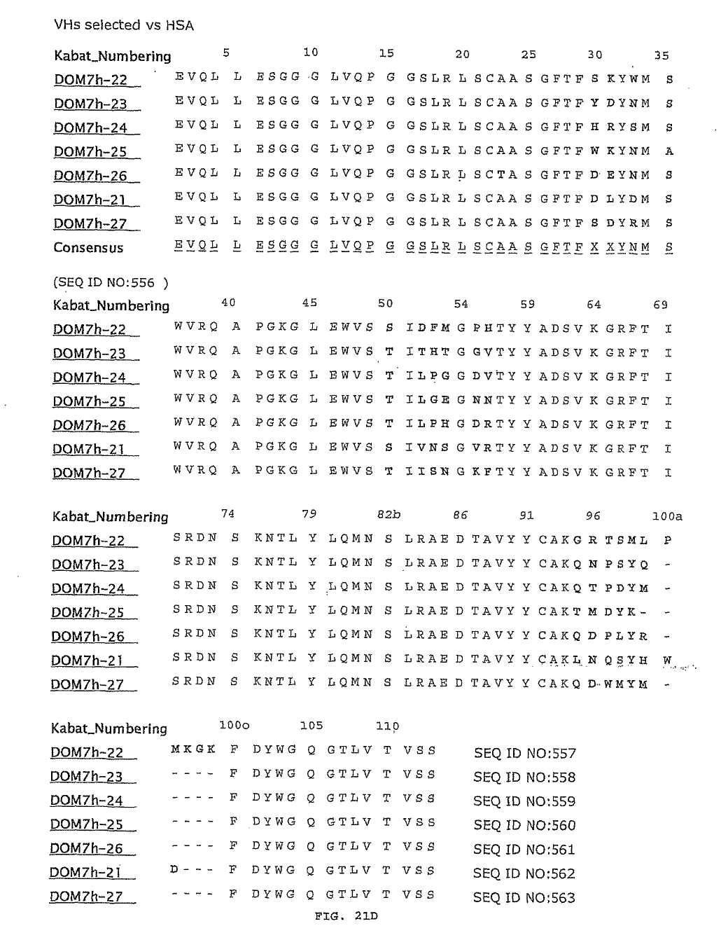

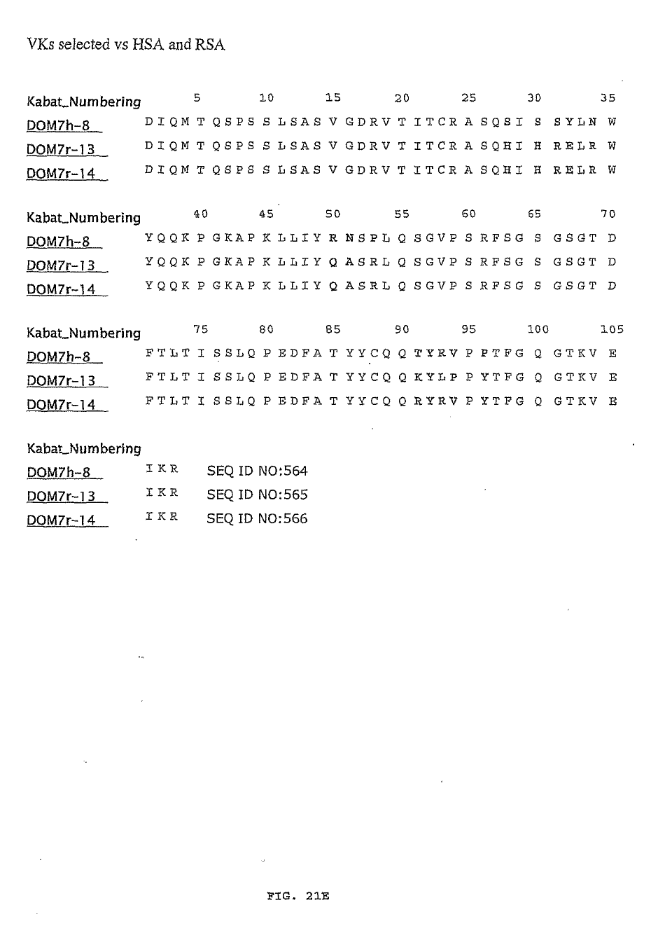

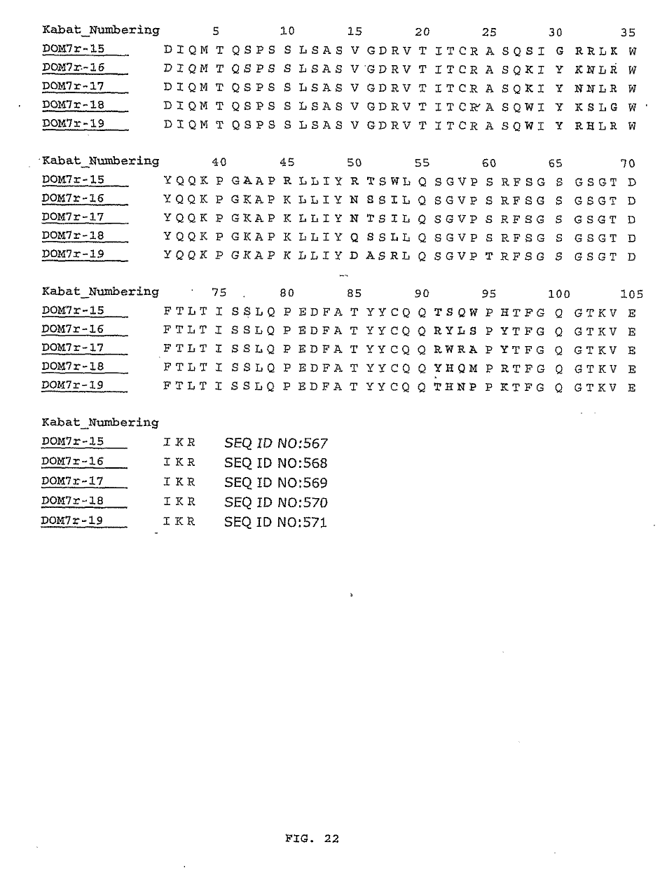

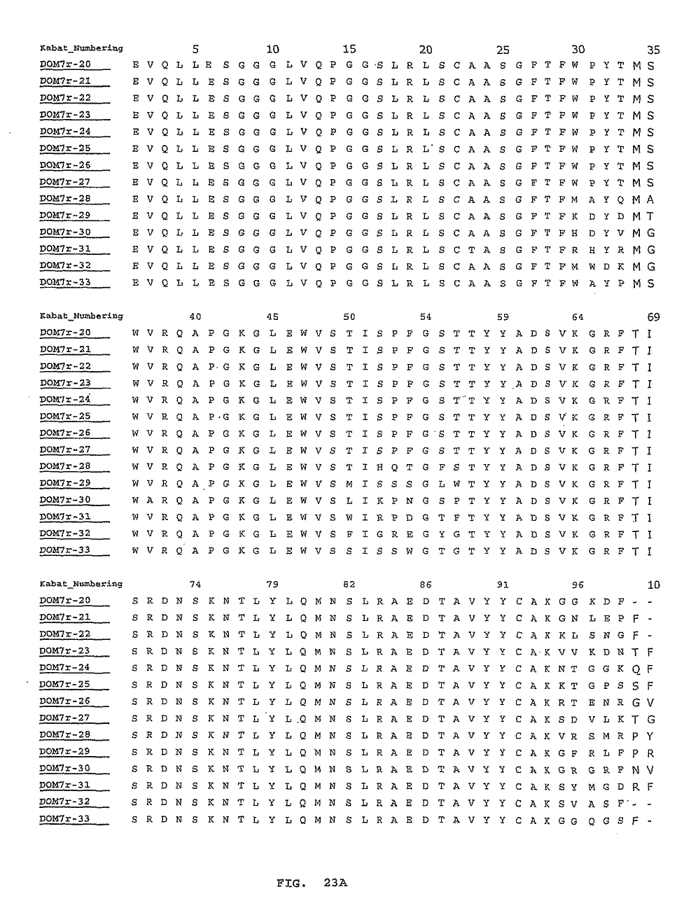

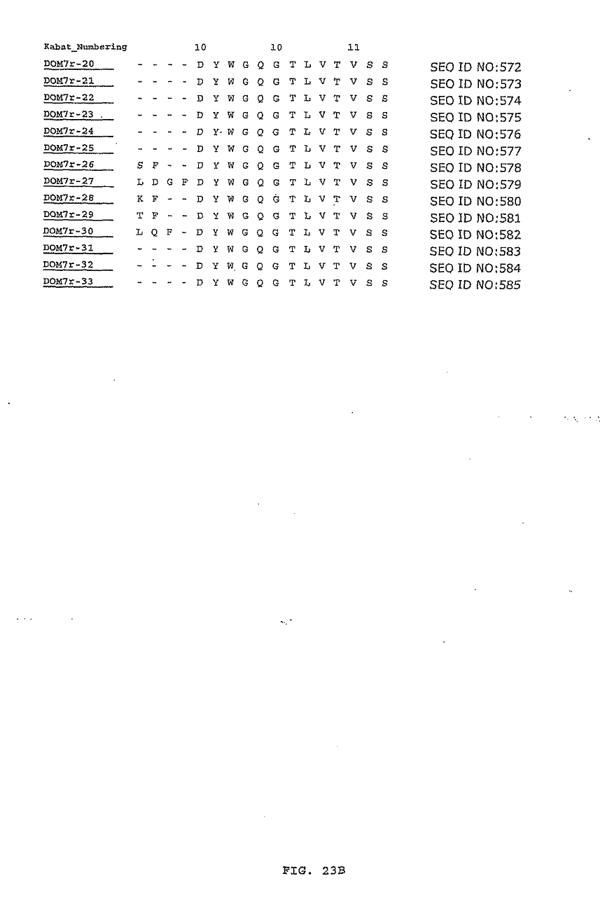

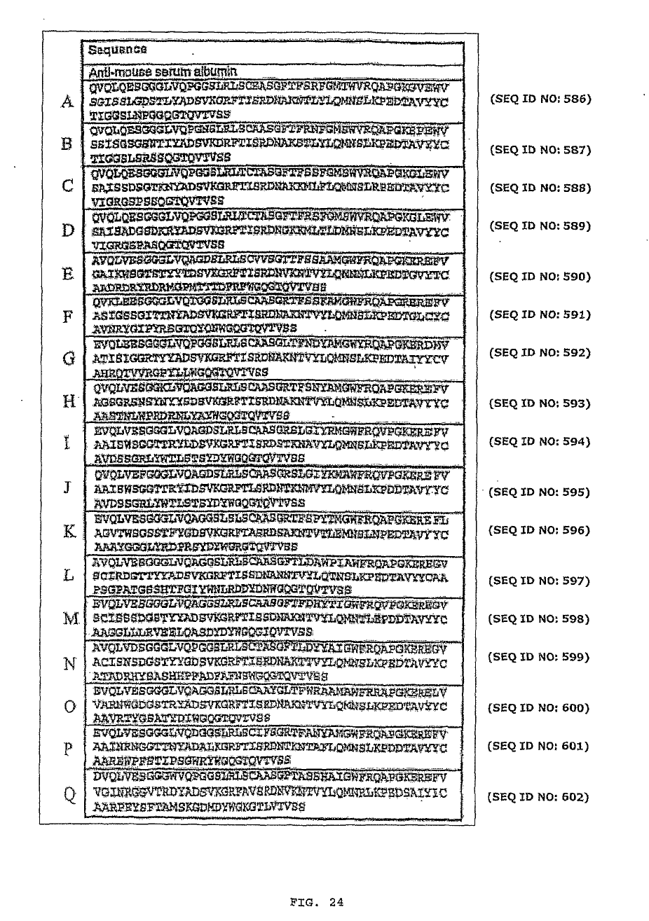

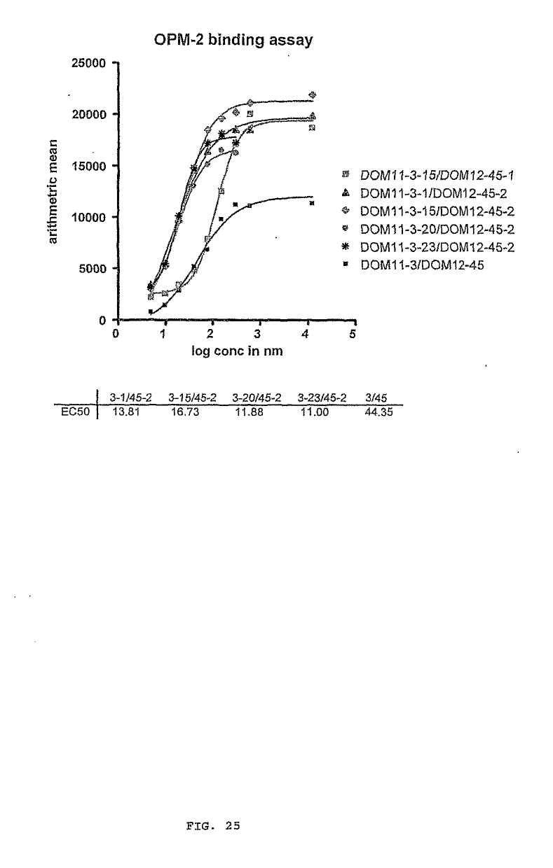

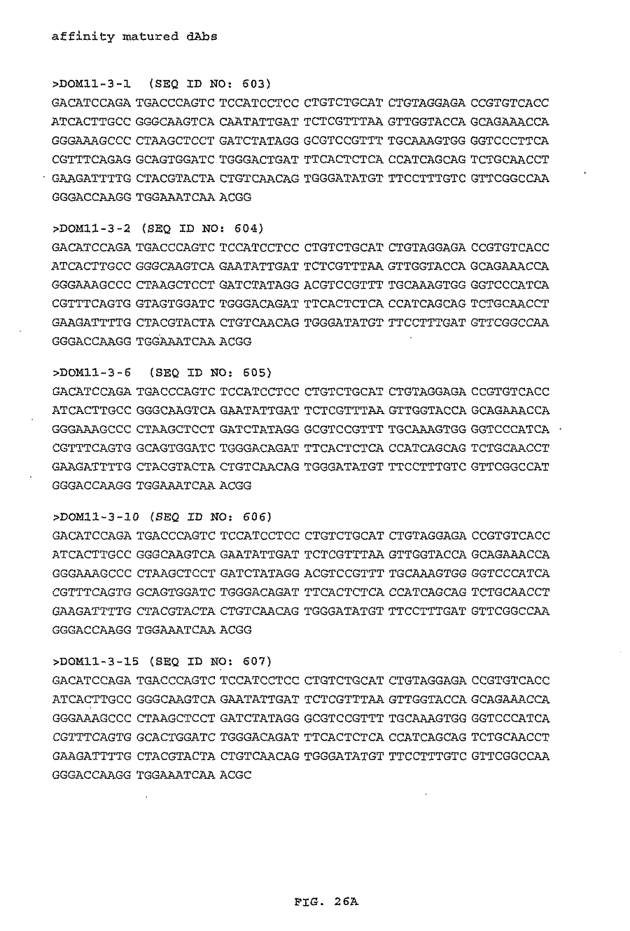

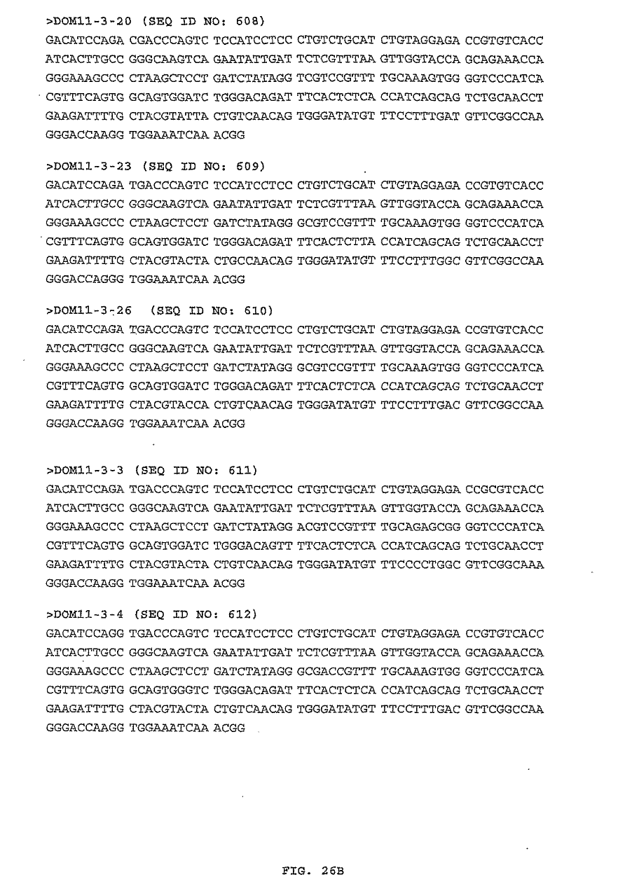

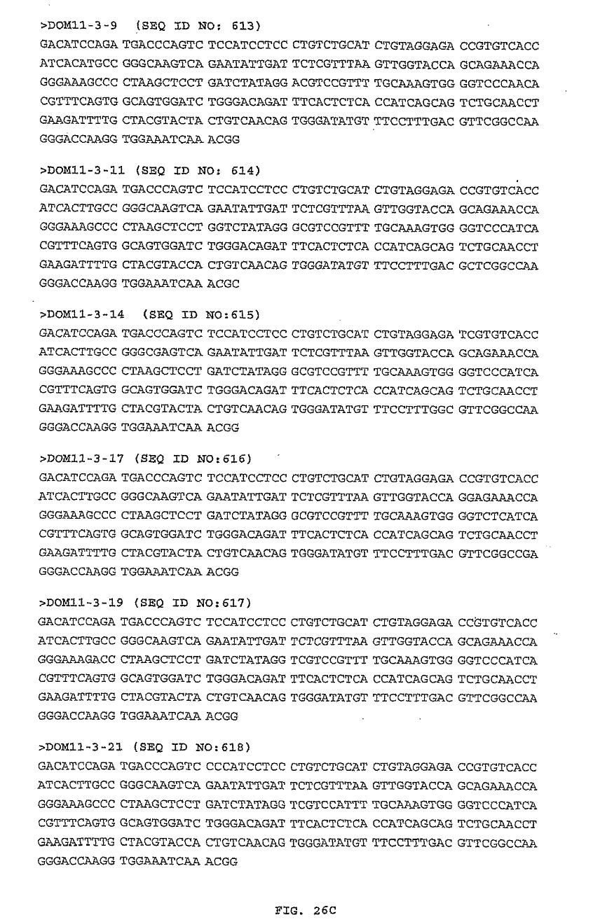

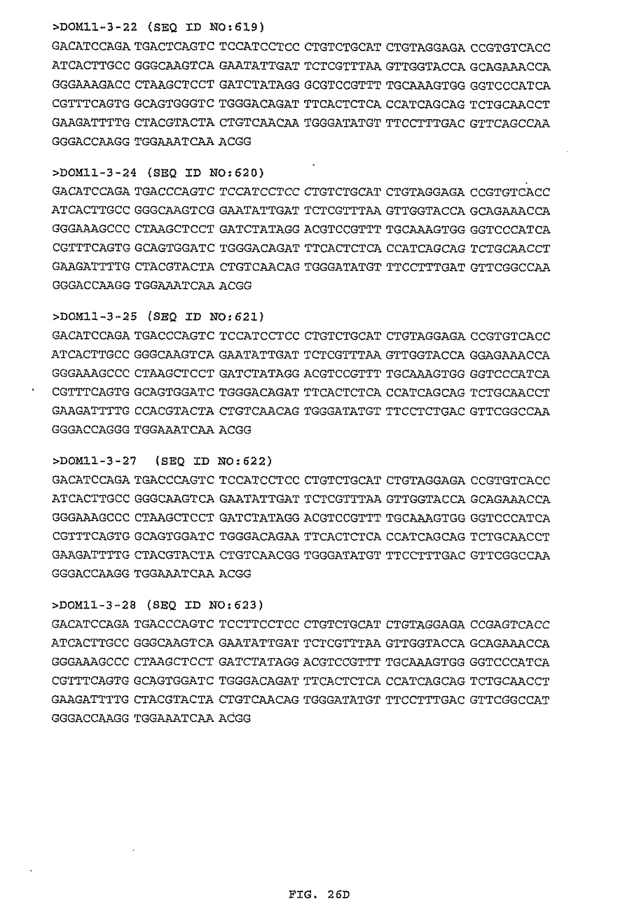

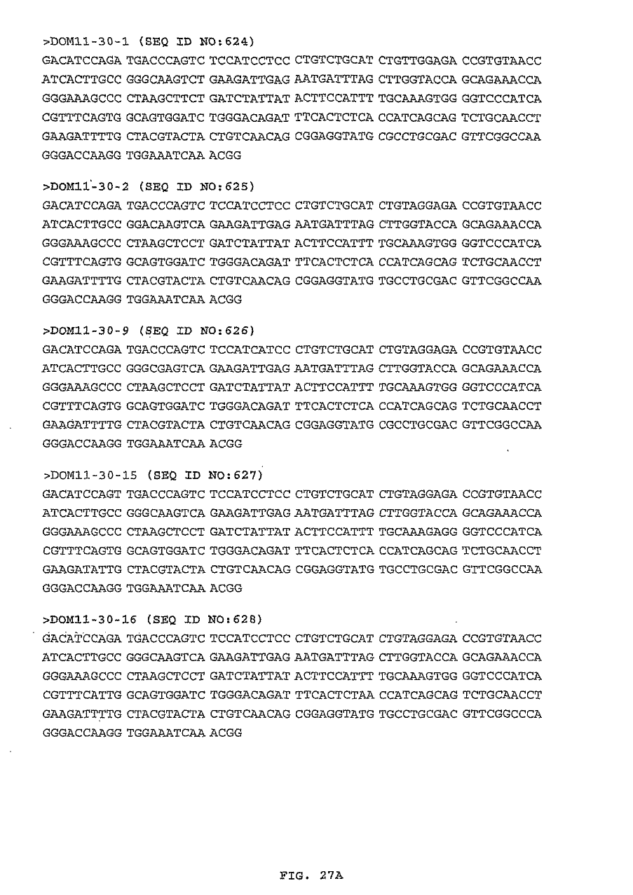

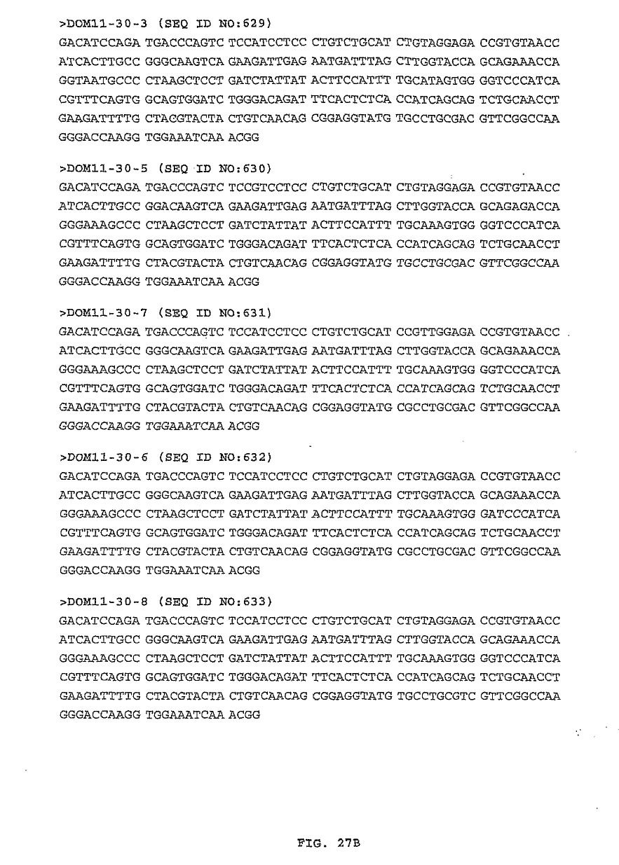

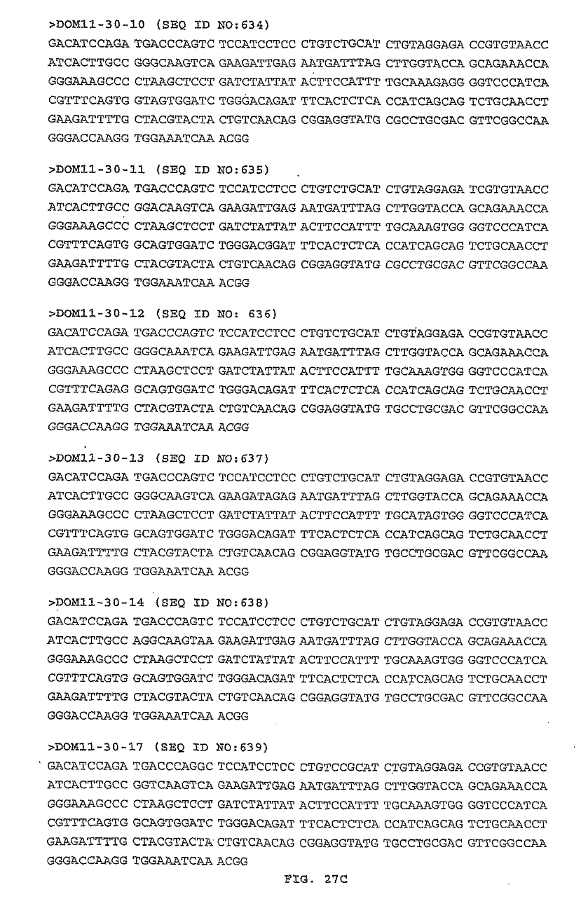

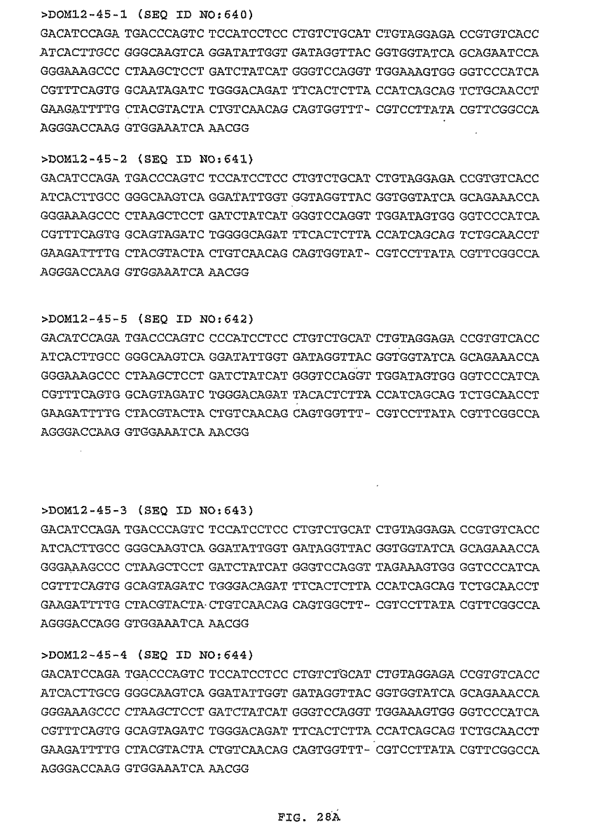

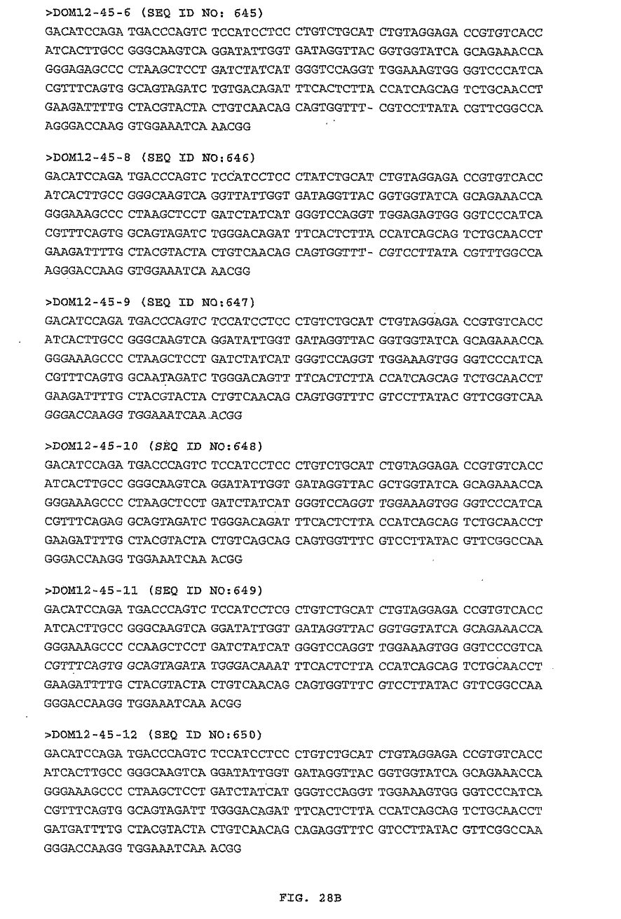

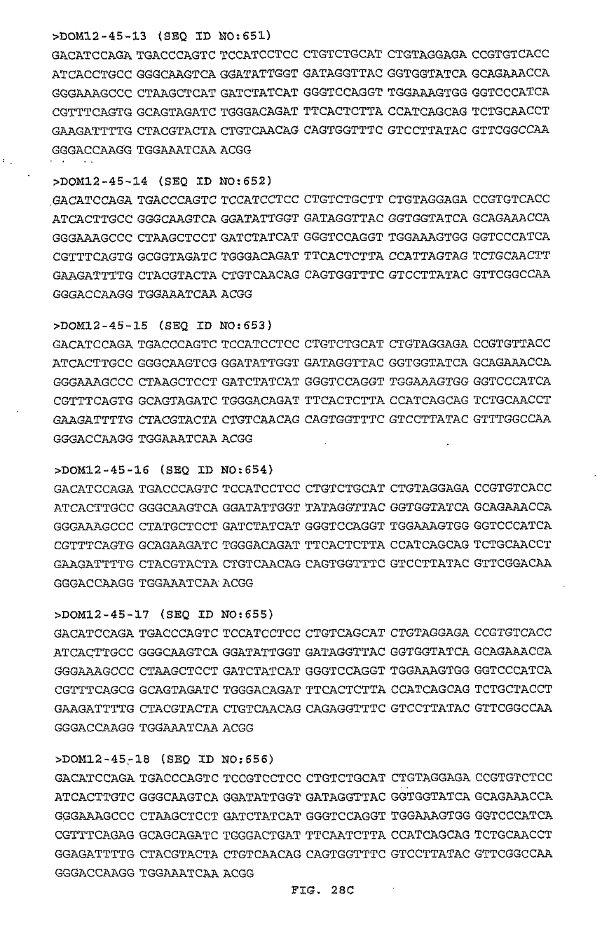

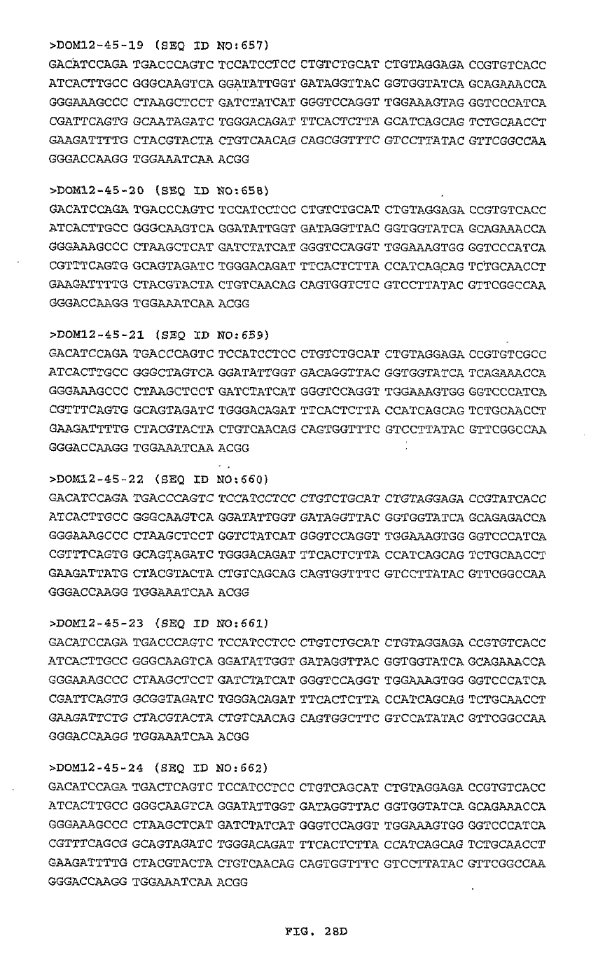

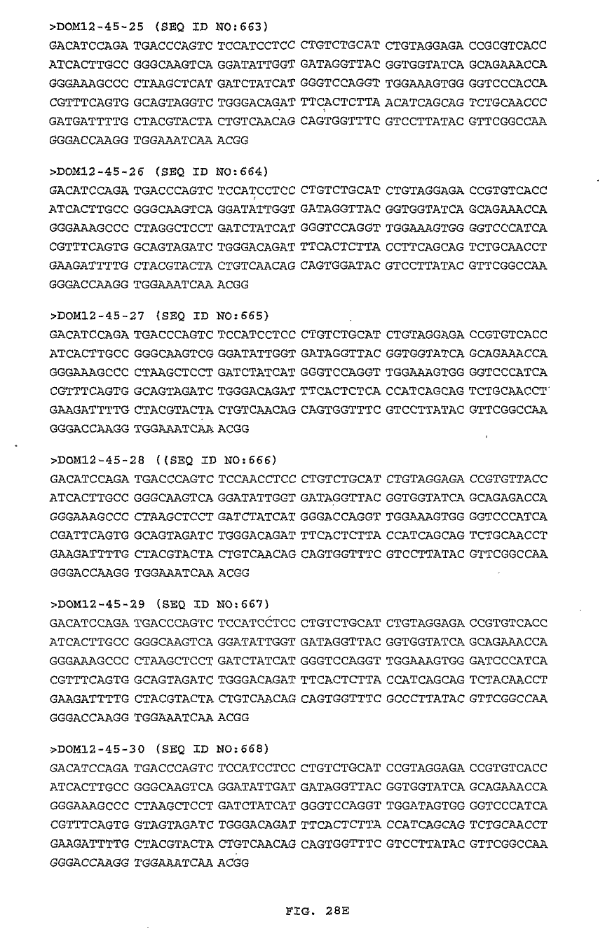

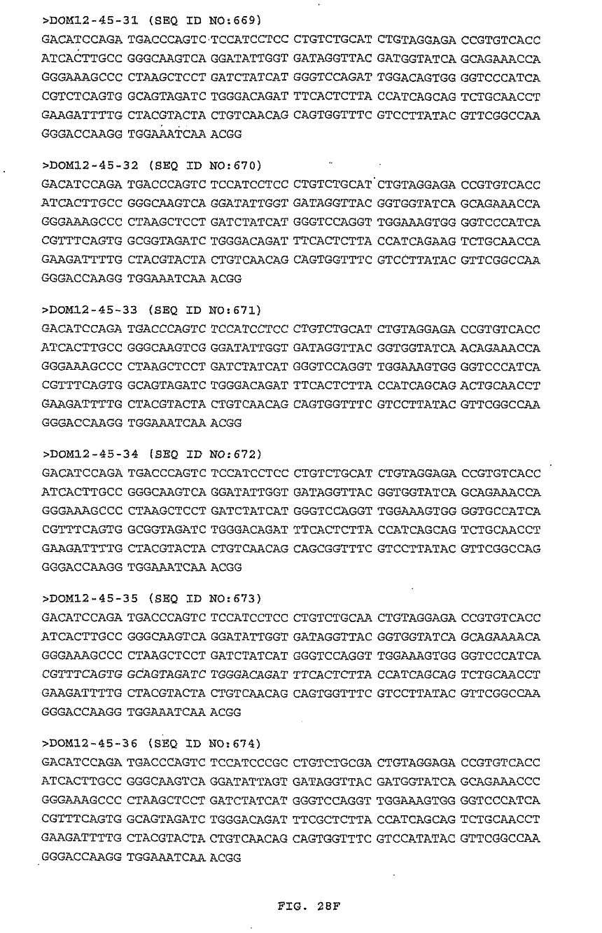

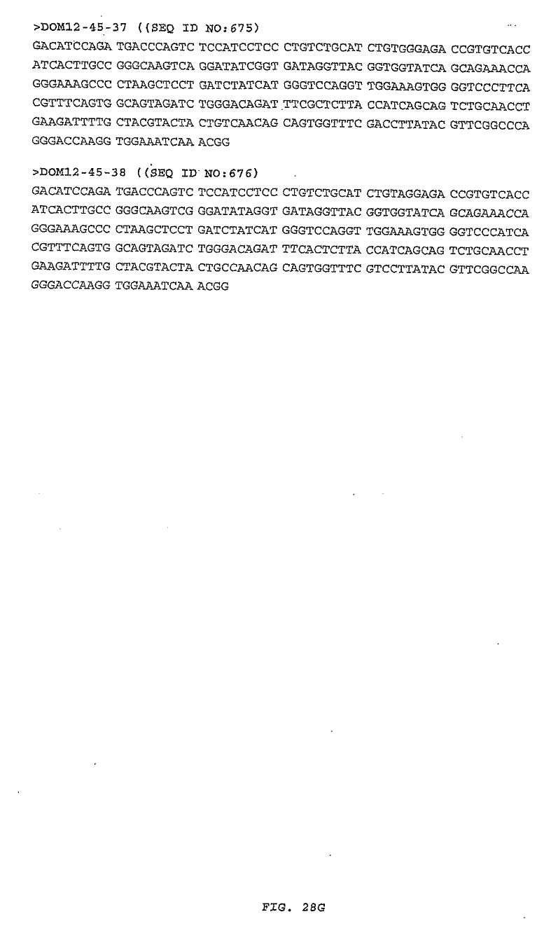

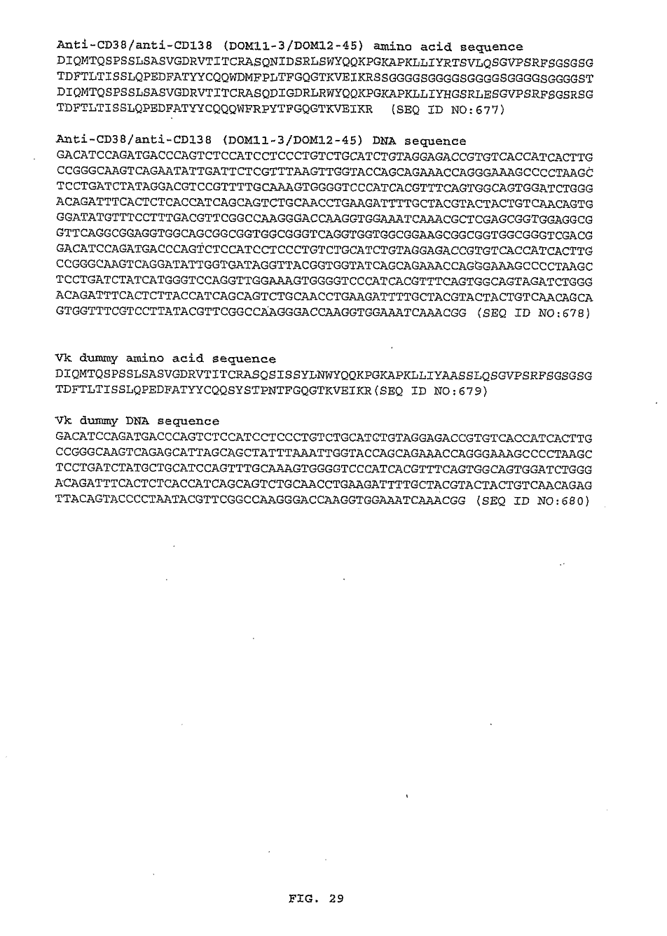

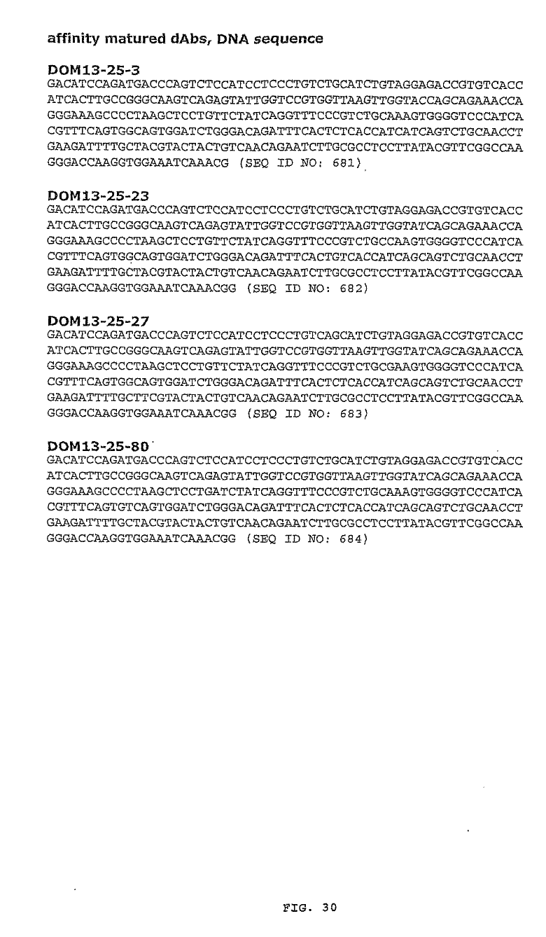

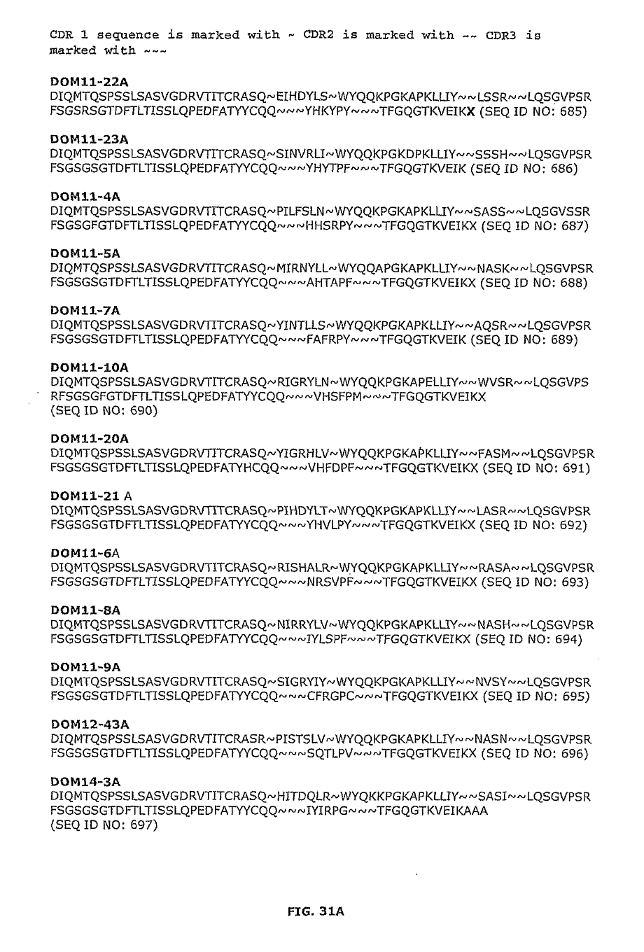

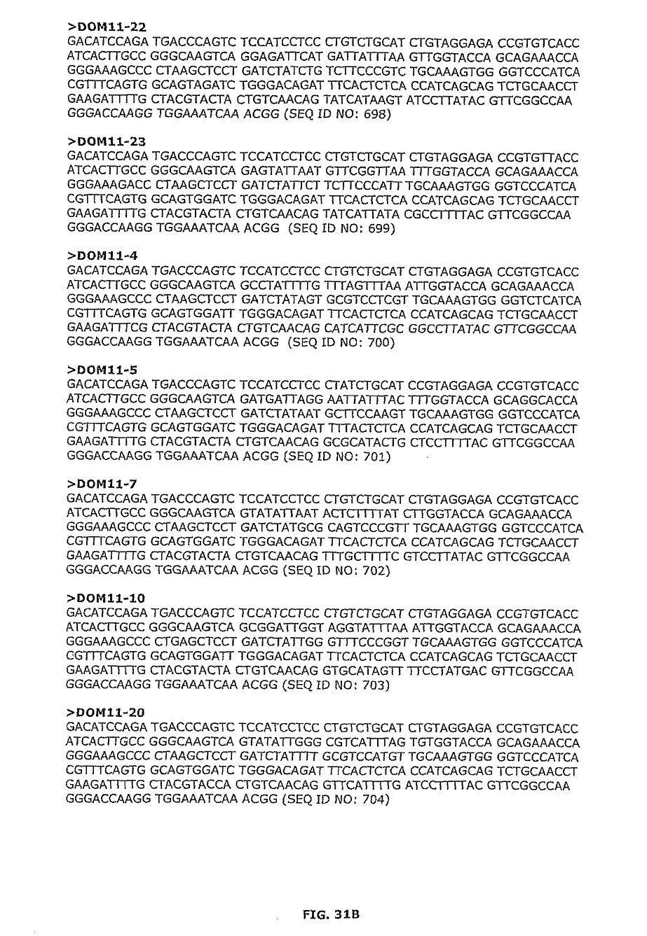

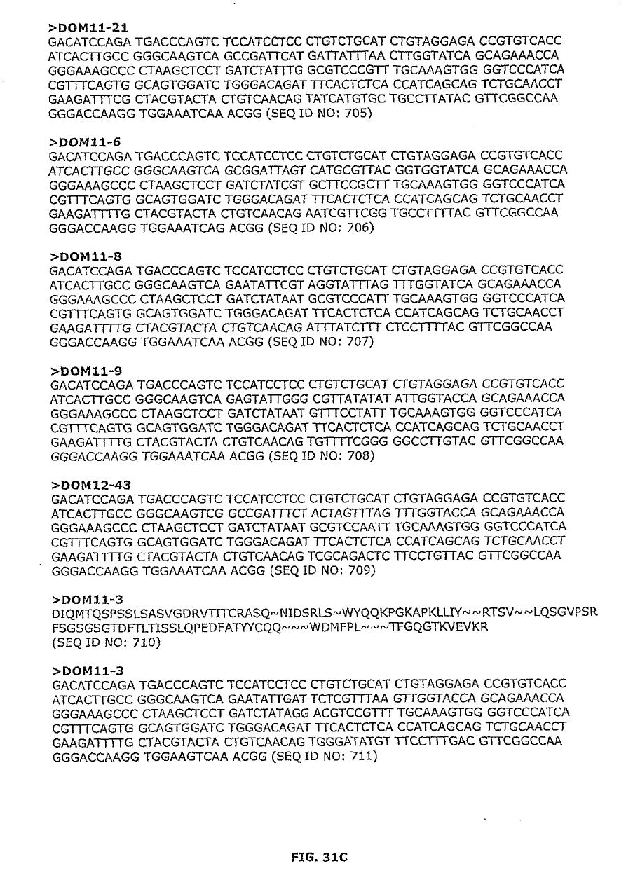

To determine the effect of the dual specific ligands-Se conjugates on apoptosis and cell death, dual staining with Aimexin V alexa-fluor 488 and propidium iodide (PI) was carried out (Vybrant Apoptosis assay kit#2, Molecular Probes). 1×10⁵ OPM2 CD38/CD138 positive multiple myeloma cells (ATCC) or CD138/CD38 antigen negative cells were incubated with dual specific dAb or Vk-dummy with and without conjugation to Selenium for 24 hours. As a positive control, cells were incubated with μM camptothecin (Sigma) for 6 hours. After treatment, the cells were washed with FACS buffer and resuspended in binding buffer containing Annexin V and propidium iodide according to manufacturer's instructions. Following incubation for 15 minutes, cells were assayed by FACS for the presence of apoptotic and dead cell populations. (As shown in FIG. 10)

The results shown is FIG. 10 demonstrate that conjugation of selenium to the dual specific anti-CD38/anti-CD138 dAb provided selective cell killing of double positive (CD38+/CD138+) cells. An increase in apoptosis on multiple myeloma cells expressing both CD38 and CD138 compared to dual specific dAb without selenium conjugation was observed. Moreover, this increase in apoptosis was specific to multiple myeloma cells that expressed both CD38 and CD138. No increase in apoptosis is observed with a negative control dAb conjugated with selenium on either CD38/CD138 positive or negative cell lines.

The effect of the ligand-Se conjugates on cell viability, 1×10⁵ OPM2 (CD38+/CD138+) multiple myeloma cells was analyzed. Raji cells (CD38 positive/CD138 negative) or CD138−/CD38− negative cells were incubated with dual specific ligand or Vk-dummy with and without conjugation to Selenium for 24 hours as described above. Cells were washed and stained with propidium iodide and the cell viability determined by FACS. The results show that conjugation of selenium to the dual specific ligand results in a reduction in cell viability on double positive multiple myeloma cells, whereas, single positive and double negative cell lines were unaffected. See FIG. 11.

In some studies, the dual specific ligands or Vk-dummy with and without conjugation were incubated with cells for 24-96 hours.

Ligands that Contain an Anti-CD138 dAb and an Anti-CD56 dAb

Low affinity dAbs have been identified that bind CD138 or CD56. The dAbs DOM12-45 and DOM14-23 have been then been linked to create dual specific dAbs that bind specifically to target expressing cells by FACS. All dAbs were expressed in E. coli and purified using protein L agarose followed by Resource S cation exchange chromatography when required

An anti-CD138/anti-CD56 dual specific ligand (DOM12-45/DOM14-23) has been made as an inline fusion. This is an alternative pairing to the anti-CD38/anti-CD138 ligands for treating multiple myeloma. It had been shown by FACS to bind strongly to double positive cell lines (CD138+/CD56+) but not to single positive or negative cell lines. DOM14-23/DOM12-45 has been shown to internalise on the double positive multiple myeloma cell line OPM2 (see Table 7).

Ligands that Contain an Anti-CEA dAb and an Anti-CD56 dAb

Low affinity dAbs have been identified that bind CEA or CD56. The dAbs (DOM13-25 and DOM14-23) have been linked to create dual specific dAbs that bind specifically to target expressing cells by FACS. All dAbs were expressed in E. coli and purified using protein L agarose followed by Resource S cation exchange chromatography when required

An anti-CEA/anti-CD56 dual specific ligand (DOM13-25/DOM14-23) has been made as an inline fusion. This ligand can be used to treat small cell lung carcinoma. It had been shown by FACS to bind strongly to the double positive cell line (H69a small cell lung carcinoma that is CEA+/CD56+) but not to single positive or negative cell lines. In addition, DOM13-25 and DOM14-23 have been paired with Vk dummy (a dAb comprising a germline amino acid sequence that does not bind CD38, CD138, CEA or CD56). When paired with Vk dummy neither dAb shows significant binding to H69 cells, only when paired together as a dual specific dAb did they bind effectively to H69 cells.

Ligands that Contain an Anti-CEA dAb and an Anti-Cd56 (DOM13/DOM14) Methods

The anti-CEA dAb, DOM13-25, and the anti-CD56 dAb, DOM14-23, were formatted as an inline fusion. This ligand is indicated for small cell lung carcinoma. It had been shown by FACS to bind strongly to double antigen positive cell lines (H69 small cell lung carcinoma, ATCC) but not to single antigen positive or negative cell lines. In addition DOM13-25 and DOM14-23 have been paired with Vk dummy. When paired with Vk dummy neither dAb shows significant binding to H69 cells only when paired together as a dual targeting dAb do they bind effectively to H69 cells.

Affinity Matured Anti-CD38 (DOM11) dAbs

Affinity maturation libraries were created for the anti-CD38 dAbs DOM11-3 and DOM11-30 by error prone PCR. Three rounds of selection were carried out on CD38-Fc antigen. dAbs from rounds 2 and 3 were shown to bind specifically by phage ELISA and subsequently by soluble ELISA (as described above). Initial screening was carried out by BIAcore (as described previously) and subsequently by FACS.

Some clones were identified that showed improved binding to antigen by BIAcore and FACS. Table 8 and Table 9 show the affinity (KD) observed for the parental dAbs and for several affinity matured anti-CD38 dAbs (DOM11-3-1, DOM11-3-2, DOM11-30-1, DOM11-30-2, DOM11-30-3, and DOM11-30-4). The affinity matured dAbs from DOM 11-30 showed improved binding affinity of up to approximately 10 fold.

TABLE 8

| CLONE | KD (nM) |
|---|---|
| DOM11-3 | 330-500 |
| DOM11-3-1 | 62 |
| DOM11-3-2 | 130-160 |

TABLE 9

| CLONE | KD (nM) |
|---|---|
| DOM11-30 | 190-230 |
| DOM11-30-1 | 19 |
| DOM11-30-2 | 62-76 |
| DOM11-30-3 | 86-93 |
| DOM11-30-4 | 78-89 |

Affinity Matured Anti-CD138 dAbs

An affinity maturation library was created for the anti-CD138 dAb DOM12-45 by error prone PCR. Three rounds of selection were carried out on CD138-Fc antigen. dAbs from rounds 2 and 3 were shown to bind specifically by phage ELSIA and subsequently by soluble ELISA. Initial screening was carried out by FACS. Lead clones were identified that showed improved binding to antigen in FACS. Affinity matured dAbs showed improved binding affinity of up to approximately 10 fold.

### Affinity Matured Anti-CD38/Anti-CD138 Dual Specific Ligands

Anti-CD38 and anti-CD138 affinity matured dAbs were paired to create dual specific ligands by cloning an anti-CD38 dAb and an anti-CD138 dAb into a dual expression vector. To determine if the increased affinity of the monomers was reflected in increased binding affinity of the dual specific ligand, a range of the affinity matured anti-CD38 dAbs were paired with the anti-CD138 dAb DOM12-45, a range of affinity matured anti-CD138 dAbs were paired with anti-CD38 dAbs, and a range of affinity matured anti-CD38 dAbs and affinity matured anti-CD138 dAbs were paired. All dual specific ligands were expressed in E. coli and purified using protein L agarose followed by Resource S cation exchange chromatography when required. The binding affinity of the dual specific ligands was assessed by FACS as described previously. In this study, a range of concentrations was used to allow determination of EC50. Results of some of the pairings are shown in FIG. 25.

While this invention has been particularly shown and described with references to preferred embodiments thereof, it will be understood by those skilled in the art that various changes in form and details may be made therein without departing from the scope of the invention encompassed by the appended claims.

1. A ligand comprising a first polypeptide domain having a binding site with binding specificity for a first cell surface target and a second polypeptide domain having a binding site with binding specificity for a second cell surface target,
wherein said first cell surface target and said second cell surface target are different, and said first cell surface target and said second cell surface target are present on a pathogenic cell;
wherein said ligand binds said first cell surface target and said second cell surface target on said pathogenic cell; and
wherein said ligand is internalized by said pathogenic cell.

2. The ligand of claim 1, wherein said ligand is preferentially internalized by said pathogenic cell.

3. The ligand of claim 1, wherein said ligand is not substantially internalized by single positive or normal cells.

4. The ligand of claim 1, wherein said ligand selectively binds said pathogenic cell.

5. The ligand of claim 1, wherein said first polypeptide domain binds said first cell surface target with low affinity and said second polypeptide domain binds said second cell surface target with low affinity.

6. The ligand of claim 5, wherein said first polypeptide domain and said second polypeptide domain each bind their respective cell surface targets with an affinity (KD) that is between about 10 μM and about 10 nM, as determined by surface plasmon resonance.

7. The ligand of claim 4, wherein said ligand selectively binds said pathogenic cell when said ligand is present at a concentration that is between about 1 μM and about 150 nM.

8. The ligand of claim 1, wherein the first polypeptide domain having a binding site with binding specificity for a first cell surface target and said second polypeptide domain having a binding site with binding specificity for a second cell surface target are a first immunoglobulin single variable domain, and a second immunoglobulin single variable domain, respectively.

9. The ligand of claim 8, wherein said first immunoglobulin single variable domain and/or said second immunoglobulin single variable domain is a V_{HH}.

10. The ligand of claim 8, wherein said first immunoglobulin single variable domain and said second immunoglobulin single variable domain are independently selected from the group consisting of a human V_{H} and a human V_{L}.

11. The ligand of claim 8, wherein said first immunoglobulin single variable domain has a binding site with binding specificity for a cell surface target selected from the group consisting of CD38, CD138, carcinoembrionic antigen (CEA), CD56, vascular endothelial growth factor (VEGF), epidermal growth factor receptor (EGFR), and HER2.

12. The ligand of claim 11, wherein the second immunoglobulin single variable domain has a binding site with binding specificity for a cell surface target selected from the group consisting of CD38, CD138, CEA, CD56, VEGF, EGFR, and HER2, with the proviso that said first immunoglobulin single variable domain and said second immunoglobulin single variable domain do not bind the same cell surface target.

13. The ligand of claim 11, wherein said first immunoglobulin single variable domain or said second immunoglobulin single variable domain binds CD38 and competes for binding to CD38 with an anti-CD38 domain antibody (dAb) selected from the group consisting of: DOM11-14 (SEQ ID NO: 242), DOM11-22 (SEQ ID NO:246), DOM11-23 (SEQ ID NO:247), DOM11-25 (SEQ ID NO:249), DOM11-26 (SEQ ID NO:250), DOM11-27 (SEQ ID NO:251), DOM 11-29 (SEQ ID NO:253), DOM11-3A (SEQ ID NO:234), DOM11-30 (SEQ ID NO:254), DOM11-31 (SEQ ID NO:255), DOM11-32 (SEQ ID NO:256), DOM11-36 (SEQ ID NO:260), DOM11-4 (SEQ ID NO:235), DOM11-43 (SEQ ID NO:266), DOM11-44 (SEQ ID NO:267), DOM11-45 (SEQ ID NO:268), DOM11-5 (SEQ ID NO:236), DOM11-7 (SEQ ID NO:238), DOM11-1 (SEQ ID NO:232), DOM11-10 (SEQ ID NO:241), DOM11-16 (SEQ ID NO:243), DOM11-2 (SEQ ID NO:233), DOM11-20 (SEQ ID NO:244), DOM11-21 (SEQ ID NO:245), DOM11-24 (SEQ ID NO:248), DOM11-28 (SEQ ID NO:252), DOM11-33 (SEQ ID NO:257), DOM11-34 (SEQ ID NO:258), DOM11-35 (SEQ ID NO:259), DOM11-37 (SEQ ID NO:261), DOM11-38 (SEQ ID NO:262), DOM11-39 (SEQ ID NO:263), DOM11-41 (SEQ ID NO:264), DOM11-42 (SEQ ID NO:265), DOM11-6 (SEQ ID NO:237), DOM11-8 (SEQ ID NO:239), and DOM11-9 (SEQ ID NO:240).

14. The ligand of claim 11, wherein said first immunoglobulin single variable domain or said second immunoglobulin single variable domain binds CD38 and competes for binding to CD38 with an anti-CD38 domain antibody (dAb) selected from the group consisting of: DOM 11-3-1 (SEQ ID NO: 269), DOM 11-3-2 (SEQ ID NO: 270), DOM 11-3-3 (SEQ ID NO: 271), DOM 11-3-4 (SEQ ID NO: 272), DOM 11-3-6 (SEQ ID NO: 273), DOM 11-3-9 (SEQ ID NO: 274), DOM 11-3-10 (SEQ ID NO: 275), DOM 11-3-11 (SEQ ID NO: 276), DOM 11-3-14 (SEQ ID NO: 277), DOM 11-3-15 (SEQ ID NO: 278), DOM 11-3-17 (SEQ ID NO: 279), DOM 11-3-19 (SEQ ID NO: 280), DOM 11-3-20 (SEQ ID NO: 281), DOM 11-3-21 (SEQ ID NO: 282), DOM 11-3-22 (SEQ ID NO: 283), DOM 11-3-23 (SEQ ID NO: 284), DOM 11-3-24 (SEQ ID NO: 285), DOM 11-3-25 (SEQ ID NO: 286), DOM 11-3-26 (SEQ ID NO: 287), DOM 11-3-27 (SEQ ID NO: 288), DOM 11-3-28 (SEQ ID NO: 289), DOM 11-30-1 (SEQ ID NO: 290), DOM 11-30-2 (SEQ ID NO: 291), DOM 11-30-3 (SEQ ID NO: 292), DOM 11-30-5 (SEQ ID NO: 293), DOM 11-30-6 (SEQ ID NO: 294), DOM 11-30-7 (SEQ ID NO: 295), DOM 11-30-8 (SEQ ID NO: 296), DOM 11-30-9 (SEQ ID NO: 297), DOM 11-30-10 (SEQ ID NO: 298), DOM 11-30-11 (SEQ ID NO: 299), DOM 11-30-12 (SEQ ID NO: 300), DOM 11-30-13 (SEQ ID NO: 301), DOM 11-30-14 (SEQ ID NO: 302), DOM 11-30-15 (SEQ ID NO: 303), DOM 11-30-16 (SEQ ID NO: 304), and DOM 11-30-17 (SEQ ID NO: 305).

15. The ligand of claim 13, wherein said first immunoglobulin single variable domain or said second immunoglobulin single variable domain comprises an amino acid sequence that has at least about 90% amino acid sequence similarity with the amino acid sequence of a dAb selected from the group consisting of: DOM 11-14 (SEQ ID NO: 242), DOM11-22 (SEQ ID NO:246), DOM11-23 (SEQ ID NO:247), DOM11-25 (SEQ ID NO:249), DOM11-26 (SEQ ID NO:250), DOM11-27 (SEQ ID NO:251), DOM 11-29 (SEQ ID NO:253), DOM11-3A (SEQ ID NO:234), DOM11-30 (SEQ ID NO:254), DOM11-31 (SEQ ID NO:255), DOM11-32 (SEQ ID NO:256), DOM11-36 (SEQ ID NO:260), DOM11-4 (SEQ ID NO:235), DOM11-43 (SEQ ID NO:266), DOM11-44 (SEQ ID NO:267), DOM11-45 (SEQ ID NO:268), DOM11-5 (SEQ ID NO:236), DOM11-7 (SEQ ID NO:238), DOM11-1 (SEQ ID NO:232), DOM11-10 (SEQ ID NO:241), DOM11-16 (SEQ ID NO:243), DOM11-2 (SEQ ID NO:233), DOM11-20 (SEQ ID NO:244), DOM11-21 (SEQ ID NO:245), DOM11-24 (SEQ ID NO:248), DOM11-28 (SEQ ID NO:252), DOM11-33 (SEQ ID NO:257), DOM11-34 (SEQ ID NO:258), DOM11-35 (SEQ ID NO:259), DOM11-37 (SEQ ID NO:261), DOM11-38 (SEQ ID NO:262), DOM11-39 (SEQ ID NO:263), DOM11-41 (SEQ ID NO:264), DOM11-42 (SEQ ID NO:265), DOM11-6 (SEQ ID NO:237), DOM11-8 (SEQ ID NO:239), and DOM11-9 (SEQ ID NO:240).

16. The ligand of claim 13, wherein said first immunoglobulin single variable domain or said second immunoglobulin single variable domain comprises an amino acid sequence that has at least about 90% amino acid sequence similarity with the amino acid sequence of a dAb selected from the group consisting of: DOM 11-3-1 (SEQ ID NO: 269), DOM 11-3-2 (SEQ ID NO: 270), DOM 11-3-3 (SEQ ID NO: 271), DOM 11-3-4 (SEQ ID NO: 272), DOM 11-3-6 (SEQ ID NO: 273), DOM 11-3-9 (SEQ ID NO: 274), DOM 11-3-10 (SEQ ID NO: 275), DOM 11-3-11 (SEQ ID NO: 276), DOM 11-3-14 (SEQ ID NO: 277), DOM 11-3-15 (SEQ ID NO: 278), DOM 11-3-17 (SEQ ID NO: 279), DOM 11-3-19 (SEQ ID NO: 280), DOM 11-3-20 (SEQ ID NO: 281), DOM 11-3-21 (SEQ ID NO: 282), DOM 11-3-22 (SEQ ID NO: 283), DOM 11-3-23 (SEQ ID NO: 284), DOM 11-3-24 (SEQ ID NO: 285), DOM 11-3-25 (SEQ ID NO: 286), DOM 11-3-26 (SEQ ID NO: 287), DOM 11-3-27 (SEQ ID NO: 288), DOM 11-3-28 (SEQ ID NO: 289), DOM 11-30-1 (SEQ ID NO: 290), DOM 11-30-2 (SEQ ID NO: 291), DOM 11-30-3 (SEQ ID NO: 292), DOM 11-30-5 (SEQ ID NO: 293), DOM 11-30-6 (SEQ ID NO: 294), DOM 11-30-7 (SEQ ID NO: 295), DOM 11-30-8 (SEQ ID NO: 296), DOM 11-30-9 (SEQ ID NO: 297), DOM 11-30-10 (SEQ ID NO: 298), DOM 11-30-11 (SEQ ID NO: 299), DOM 11-30-12 (SEQ ID NO: 300), DOM 11-30-13 (SEQ ID NO: 301), DOM 11-30-14 (SEQ ID NO: 302), DOM 11-30-15 (SEQ ID NO: 303), DOM 11-30-16 (SEQ ID NO: 304), and DOM 11-30-17 (SEQ ID NO: 305).

17. The ligand of claim 11, wherein said first immunoglobulin single variable domain or said second immunoglobulin single variable domain binds CD138 and competes for binding to CD138 with an anti-CD138 domain antibody (dAb) selected from the group consisting of: DOM12-1 (SEQ ID NO:306), DOM12-15 (SEQ ID NO:317), DOM12-17 (SEQ ID NO:318), DOM12-19 (SEQ ID NO:320), DOM12-2 (SEQ ID NO:307), DOM12-20 (SEQ ID NO:321), DOM12-21 (SEQ ID NO:322), DOM12-22 (SEQ ID NO:323), DOM12-3 (SEQ ID NO:308), DOM12-33 (SEQ ID NO:334), DOM12-39 (SEQ ID NO:340), DOM12-4 (SEQ ID NO:309), DOM12-40 (SEQ ID NO:341), DOM12-41 (SEQ ID NO:342), DOM12-42 (SEQ ID NO:343), DOM12-44 (SEQ ID NO:345), DOM12-46 (SEQ ID NO:347), DOM12-6 (SEQ ID NO:311), DOM12-7 (SEQ ID NO:312), DOM12-10 (SEQ ID NO:315), DOM12-11 (SEQ ID NO:316), DOM12-18 (SEQ ID NO:319), DOM12-23 (SEQ ID NO:324), DOM12-24 (SEQ ID NO:325), DOM12-25 (SEQ ID NO:326), DOM12-26 (SEQ ID NO:327), DOM12-27 (SEQ ID NO:328), DOM12-28 (SEQ ID NO:329), DOM12-29 (SEQ ID NO:330), DOM12-30 (SEQ ID NO:331), DOM12-31 (SEQ ID NO:332), DOM12-32 (SEQ ID NO:333), DOM12-34 (SEQ ID NO:335), DOM12-35 (SEQ ID NO:336), DOM12-36 (SEQ ID NO:337), DOM12-37 (SEQ ID NO:338), DOM12-38 (SEQ ID NO:339), DOM12-43 (SEQ ID NO:344), DOM12-45 (SEQ ID NO:346), DOM12-5 (SEQ ID NO:310), DOM12-8 (SEQ ID NO:313), and DOM12-9 (SEQ ID NO:314).

18. The ligand of claim 11, wherein said first immunoglobulin single variable domain or said second immunoglobulin single variable domain binds CD138 and competes for binding to CD138 with an anti-CD138 domain antibody (dAb) selected from the group consisting of: DOM 12-45-1 (SEQ ID NO: 348), DOM 12-45-2 (SEQ ID NO: 349), DOM 12-45-3 (SEQ ID NO: 350), DOM 12-45-4 (SEQ ID NO: 351), DOM 12-45-5 (SEQ ID NO: 352), DOM 12-45-6 (SEQ ID NO: 353), DOM 12-45-8 (SEQ ID NO: 354), DOM 12-45-9 (SEQ ID NO: 355), DOM 12-45-10 (SEQ ID NO: 356), DOM 12-45-11 (SEQ ID NO: 357), DOM 12-45-12 (SEQ ID NO: 358), DOM 12-45-13 (SEQ ID NO: 359), DOM 12-45-14 (SEQ ID NO: 360), DOM 12-45-15 (SEQ ID NO: 361), DOM 12-45-16 (SEQ ID NO: 362), DOM 12-45-17 (SEQ ID NO: 363), DOM 12-45-18 (SEQ ID NO: 364), DOM 12-45-19 (SEQ ID NO: 365), DOM 12-45-20 (SEQ ID NO: 366), DOM 12-45-21 (SEQ ID NO: 367), DOM 12-45-22 (SEQ ID NO: 368), DOM 12-45-23 (SEQ ID NO: 369), DOM 12-45-24 (SEQ ID NO: 370), DOM 12-45-25 (SEQ ID NO: 371), DOM 12-45-26 (SEQ ID NO: 372), DOM 12-45-27 (SEQ ID NO: 373), DOM 12-45-28 (SEQ ID NO: 374), DOM 12-45-29 (SEQ ID NO: 375), DOM 12-45-30 (SEQ ID NO: 376), DOM 12-45-31 (SEQ ID NO: 377), DOM 12-45-32 (SEQ ID NO: 378), DOM 12-45-33 (SEQ ID NO: 379), DOM 12-45-34 (SEQ ID NO: 380), DOM 12-45-35 (SEQ ID NO: 381), DOM 12-45-36 (SEQ ID NO: 382), DOM 12-45-37 (SEQ ID NO: 383), and DOM 12-45-38 (SEQ ID NO: 384).

19. The ligand of claim 17, wherein said first immunoglobulin single variable domain or said second immunoglobulin single variable domain comprises an amino acid sequence that has at least about 90% amino acid sequence similarity with the amino acid sequence of a dAb selected from the group consisting of: DOM12-1 (SEQ ID NO:306), DOM12-15 (SEQ ID NO:317), DOM12-17 (SEQ ID NO:318), DOM12-19 (SEQ ID NO:320), DOM12-2 (SEQ ID NO:307), DOM12-20 (SEQ ID NO:321), DOM12-21 (SEQ ID NO:322), DOM12-22 (SEQ ID NO:323), DOM12-3 (SEQ ID NO:308), DOM12-33 (SEQ ID NO:334), DOM12-39 (SEQ ID NO:340), DOM12-4 (SEQ ID NO:309), DOM12-40 (SEQ ID NO:341), DOM12-41 (SEQ ID NO:342), DOM12-42 (SEQ ID NO:343), DOM12-44 (SEQ ID NO:345), DOM12-46 (SEQ ID NO:347), DOM12-6 (SEQ ID NO:311), DOM12-7 (SEQ ID NO:312), DOM12-10 (SEQ ID NO:315), DOM12-11 (SEQ ID NO:316), DOM12-18 (SEQ ID NO:319), DOM12-23 (SEQ ID NO:324), DOM12-24 (SEQ ID NO:325), DOM12-25 (SEQ ID NO:326), DOM12-26 (SEQ ID NO:327), DOM12-27 (SEQ ID NO:328), DOM12-28 (SEQ ID NO:329), DOM12-29 (SEQ ID NO:330), DOM12-30 (SEQ ID NO:331), DOM12-31 (SEQ ID NO:332), DOM12-32 (SEQ ID NO:333), DOM12-34 (SEQ ID NO:335), DOM12-35 (SEQ ID NO:336), DOM12-36 (SEQ ID NO:337), DOM12-37 (SEQ ID NO:338), DOM12-38 (SEQ ID NO:339), DOM12-43 (SEQ ID NO:344), DOM12-45 (SEQ ID NO:346), DOM12-5 (SEQ ID NO:310), DOM12-8 (SEQ ID NO:313), and DOM12-9 (SEQ ID NO:314).

20. The ligand of claim 17, wherein said first immunoglobulin single variable domain or said second immunoglobulin single variable domain comprises an amino acid sequence that has at least about 90% amino acid sequence similarity with the amino acid sequence of a dAb selected from the group consisting of: DOM 12-45-1 (SEQ ID NO: 348), DOM 12-45-2 (SEQ ID NO: 349), DOM 12-45-3 (SEQ ID NO: 350), DOM 12-45-4 (SEQ ID NO: 351), DOM 12-45-5 (SEQ ID NO: 352), DOM 12-45-6 (SEQ ID NO: 353), DOM 12-45-8 (SEQ ID NO: 354), DOM 12-45-9 (SEQ ID NO: 355), DOM 12-45-10 (SEQ ID NO: 356), DOM 12-45-11 (SEQ ID NO: 357), DOM 12-45-12 (SEQ ID NO: 358), DOM 12-45-13 (SEQ ID NO: 359), DOM 12-45-14 (SEQ ID NO: 360), DOM 12-45-15 (SEQ ID NO: 361), DOM 12-45-16 (SEQ ID NO: 362), DOM 12-45-17 (SEQ ID NO: 363), DOM 12-45-18 (SEQ ID NO: 364), DOM 12-45-19 (SEQ ID NO: 365), DOM 12-45-20 (SEQ ID NO: 366), DOM 12-45-21 (SEQ ID NO: 367), DOM 12-45-22 (SEQ ID NO: 368), DOM 12-45-23 (SEQ ID NO: 369), DOM 12-45-24 (SEQ ID NO: 370), DOM 12-45-25 (SEQ ID NO: 371), DOM 12-45-26 (SEQ ID NO: 372), DOM 12-45-27 (SEQ ID NO: 373), DOM 12-45-28 (SEQ ID NO: 374), DOM 12-45-29 (SEQ ID NO: 375), DOM 12-45-30 (SEQ ID NO: 376), DOM 12-45-31 (SEQ ID NO: 377), DOM 12-45-32 (SEQ ID NO: 378), DOM 12-45-33 (SEQ ID NO: 379), DOM 12-45-34 (SEQ ID NO: 380), DOM 12-45-35 (SEQ ID NO: 381), DOM 12-45-36 (SEQ ID NO: 382), DOM 12-45-37 (SEQ ID NO: 383), and DOM 12-45-38 (SEQ ID NO: 384).

21. The ligand of claim 11, wherein said first immunoglobulin single variable domain or said second immunoglobulin single variable domain binds CEA and competes for binding to CEA with an anti-CEA domain antibody (dAb) selected from the group consisting of: DOM13-1 (SEQ ID NO:385), DOM13-12 (SEQ ID NO:393), DOM13-13 (SEQ ID NO:394), DOM13-14 (SEQ ID NO:395), DOM13-15 (SEQ ID NO:396), DOM13-16 (SEQ ID NO:397), DOM13-17 (SEQ ID NO:398), DOM13-18 (SEQ ID NO:399), DOM13-19 (SEQ ID NO:400), DOM13-2 (SEQ ID NO:386), DOM13-20 (SEQ ID NO:401), DOM13-21 (SEQ ID NO:402), DOM13-22 (SEQ ID NO:403), DOM13-23 (SEQ ID NO:404), DOM13-24 (SEQ ID NO:405), DOM13-25 (SEQ ID NO:406), DOM13-26 (SEQ ID NO:407), DOM13-27 (SEQ ID NO:408), DOM13-28 (SEQ ID NO:409), DOM13-29 (SEQ ID NO:410), DOM13-3 (SEQ ID NO:387), DOM13-30 (SEQ ID NO:411), DOM13-31 (SEQ ID NO:412), DOM13-32 (SEQ ID NO:413), DOM13-33 (SEQ ID NO:414), DOM-13-34 (SEQ ID NO:415), DOM13-35 (SEQ ID NO:416), DOM13-36 (SEQ ID NO:417), DOM13-37 (SEQ ID NO:418), DOM13-4 (SEQ ID NO:388), DOM13-42 (SEQ ID NO:419), DOM13-43 (SEQ ID NO:420), DOM13-44 (SEQ ID NO:421), DOM13-45 (SEQ ID NO:422), DOM13-46 (SEQ ID NO:423), DOM13-47 (SEQ ID NO:424), DOM13-48 (SEQ ID NO:425), DOM13-49 (SEQ ID NO:426), DOM13-5 (SEQ ID NO:389), DOM13-50 (SEQ ID NO:427), DOM13-51 (SEQ ID NO:428), DOM13-52 (SEQ ID NO:429), DOM13-53 (SEQ ID NO:430), DOM13-54 (SEQ ID NO:431), DOM13-55 (SEQ ID NO:432), DOM13-56 (SEQ ID NO:433), DOM13-57 (SEQ ID NO:434), DOM13-58 (SEQ ID NO:435), DOM13-59 (SEQ ID NO:436), DOM13-6 (SEQ ID NO:390), DOM13-60 (SEQ ID NO:437), DOM13-61 (SEQ ID NO:438), DOM13-62 (SEQ ID NO:439), DOM13-63 (SEQ ID NO:440), DOM13-64 (SEQ ID NO:441), DOM13-65 (SEQ ID NO:442), DOM13-66 (SEQ ID NO:443), DOM13-67 (SEQ ID NO:444), DOM13-68 (SEQ ID NO:445), DOM13-69 (SEQ ID NO:446), DOM13-7 (SEQ ID NO:391), DOM13-70 (SEQ ID NO:447), DOM13-71 (SEQ ID NO:448), DOM13-72 (SEQ ID NO:449), DOM13-73 (SEQ ID NO:450), DOM13-74 (SEQ ID NO:451), DOM13-75 (SEQ ID NO:452), DOM13-76 (SEQ ID NO:453), DOM13-77 (SEQ ID NO:454), DOM13-78 (SEQ ID NO:455), DOM13-79 (SEQ ID NO:456), DOM13-8 (SEQ ID NO:392), DOM13-80 (SEQ ID NO:457), DOM13-81 (SEQ ID NO:458), DOM13-82 (SEQ ID NO:459), DOM13-83 (SEQ ID NO:460), DOM13-84 (SEQ ID NO:461), DOM13-85 (SEQ ID NO:462), DOM13-86 (SEQ ID NO:463), DOM13-87 (SEQ ID NO:464), DOM13-88 (SEQ ID NO:465), DOM13-89 (SEQ ID NO:466), DOM13-90 (SEQ ID NO:467), DOM13-91 (SEQ ID NO:468), DOM13-92 (SEQ ID NO:469), DOM13-93 (SEQ ID NO:470), DOM13-94 (SEQ ID NO:471), and DOM13-95 (SEQ ID NO:472).

22. The ligand of claim 11, wherein said first immunoglobulin single variable domain or said second immunoglobulin single variable domain binds CEA and competes for binding to CEA with an anti-CEA domain antibody (dAb) selected from the group consisting of: DOM 13-25-3 (SEQ ID NO: 473), DOM 13-25-23 (SEQ ID NO: 474), DOM 13-25-27 (SEQ ID NO: 475), and DOM 13-25-80 (SEQ ID NO: 476).

23. The ligand of claim 21, wherein said first immunoglobulin single variable domain or said second immunoglobulin single variable domain comprises an amino acid sequence that has at least about 90% amino acid sequence similarity with the amino acid sequence of a dAb selected from the group consisting of: DOM 13-1 (SEQ ID NO:385), DOM13-12 (SEQ ID NO:393), DOM13-13 (SEQ ID NO:394), DOM13-14 (SEQ ID NO:395), DOM13-15 (SEQ ID NO:3396), DOM13-16 (SEQ ID NO:397), DOM13-17 (SEQ ID NO:398), DOM13-18 (SEQ ID NO:399), DOM13-19 (SEQ ID NO:400), DOM13-2 (SEQ ID NO:386), DOM13-20 (SEQ ID NO:401), DOM13-21 (SEQ ID NO:402), DOM13-22 (SEQ ID NO:403), DOM13-23 (SEQ ID NO:404), DOM13-24 (SEQ ID NO:405), DOM13-25 (SEQ ID NO:406), DOM13-26 (SEQ ID NO:407), DOM13-27 (SEQ ID NO:408), DOM13-28 (SEQ ID NO:409), DOM13-29 (SEQ ID NO:410), DOM13-3 (SEQ ID NO:387), DOM13-30 (SEQ ID NO:411), DOM13-31 (SEQ ID NO:412), DOM13-32 (SEQ ID NO:413), DOM13-33 (SEQ ID NO:414), DOM-13-34 (SEQ ID NO:415), DOM13-35 (SEQ ID NO:416), DOM13-36 (SEQ ID NO:417), DOM13-37 (SEQ ID NO:418), DOM13-4 (SEQ ID NO:388), DOM13-42 (SEQ ID NO:419), DOM13-43 (SEQ ID NO:420), DOM13-44 (SEQ ID NO:421), DOM13-45 (SEQ ID NO:422), DOM13-46 (SEQ ID NO:423), DOM13-47 (SEQ ID NO:424), DOM13-48 (SEQ ID NO:425), DOM13-49 (SEQ ID NO:426), DOM13-5 (SEQ ID NO:389), DOM13-50 (SEQ ID NO:427), DOM13-51 (SEQ ID NO:428), DOM13-52 (SEQ ID NO:429), DOM13-53 (SEQ ID NO:430), DOM13-54 (SEQ ID NO:431), DOM13-55 (SEQ ID NO:432), DOM13-56 (SEQ ID NO:433), DOM13-57 (SEQ ID NO:434), DOM13-58 (SEQ ID NO:435), DOM13-59 (SEQ ID NO:436), DOM13-6 (SEQ ID NO:390), DOM13-60 (SEQ ID NO:437), DOM13-61 (SEQ ID NO:438), DOM13-62 (SEQ ID NO:439), DOM13-63 (SEQ ID NO:440), DOM13-64 (SEQ ID NO:441), DOM13-65 (SEQ ID NO:442), DOM13-66 (SEQ ID NO:443), DOM13-67 (SEQ ID NO:444), DOM13-68 (SEQ ID NO:445), DOM13-69 (SEQ ID NO:446), DOM13-7 (SEQ ID NO:391), DOM13-70 (SEQ ID NO:447), DOM13-71 (SEQ ID NO:448), DOM13-72 (SEQ ID NO:449), DOM13-73 (SEQ ID NO:450), DOM13-74 (SEQ ID NO:451), DOM13-75 (SEQ ID NO:452), DOM13-76 (SEQ ID NO:453), DOM13-77 (SEQ ID NO:454), DOM13-78 (SEQ ID NO:455), DOM13-79 (SEQ ID NO:456), DOM13-8 (SEQ ID NO:392), DOM13-80 (SEQ ID NO:457), DOM13-81 (SEQ ID NO:458), DOM13-82 (SEQ ID NO:459), DOM13-83 (SEQ ID NO:460), DOM13-84 (SEQ ID NO:461), DOM13-85 (SEQ ID NO:462), DOM13-86 (SEQ ID NO:463), DOM13-87 (SEQ ID NO:464), DOM13-88 (SEQ ID NO:465), DOM13-89 (SEQ ID NO:466), DOM13-90 (SEQ ID NO:467), DOM13-91 (SEQ ID NO:468), DOM13-92 (SEQ ID NO:469), DOM13-93 (SEQ ID NO:470), DOM13-94 (SEQ ID NO:471), and DOM13-95 (SEQ ID NO:472).

24. The ligand of claim 21, wherein said first immunoglobulin single variable domain or said second immunoglobulin single variable domain comprises an amino acid sequence that has at least about 90% amino acid sequence similarity with the amino acid sequence of a dAb selected from the group consisting of: DOM 13-25-3 (SEQ ID NO: 473), DOM 13-25-23 (SEQ ID NO: 474), DOM 13-25-27 (SEQ ID NO: 475), and DOM 13-25-80 (SEQ ID NO: 476).

25. The ligand of claim 11, wherein said first immunoglobulin single variable domain or said second immunoglobulin single variable domain binds CD56 and competes for binding to CD56 with an anti-CD56 domain antibody (dAb) selected from the group consisting of: DOM14-1 (SEQ ID NO:477), DOM14-10 (SEQ ID NO:481), DOM14-100 (SEQ ID NO:540), DOM14-11 (SEQ ID NO:482), DOM14-12 (SEQ ID NO:483), DOM14-13 (SEQ ID NO:484), DOM14-14 (SEQ ID NO:485), DOM14-15 (SEQ ID NO:486), DOM14-16 (SEQ ID NO:487), DOM14-17 (SEQ ID NO:488), DOM14-18 (SEQ ID NO:489), DOM14-19 (SEQ ID NO:490), DOM14-2 (SEQ ID NO:478), DOM14-20 (SEQ ID NO:491), DOM14-21 (SEQ ID NO:492), DOM14-22 (SEQ ID NO:493), DOM14-23 (SEQ ID NO:494), DOM14-24 (SEQ ID NO:495), DOM14-25 (SEQ ID NO:496), DOM14-26 (SEQ ID NO:497), DOM14-27 (SEQ ID NO:498), DOM14-28 (SEQ ID NO:499), DOM14-3 (SEQ ID NO:479), DOM14-31 (SEQ ID NO:500), DOM14-32 (SEQ ID NO:501), DOM14-33 (SEQ ID NO:502), DOM14-34 (SEQ ID NO:503), DOM14-35 (SEQ ID NO:504), DOM14-36 (SEQ ID NO:505), DOM14-37 (SEQ ID NO:506), DOM14-38 (SEQ ID NO:507), DOM14-39 (SEQ ID NO:508), DOM14-4 (SEQ ID NO:480), DOM14-40 (SEQ ID NO:509), DOM14-41 (SEQ ID NO:510), DOM14-42 (SEQ ID NO:511), DOM14-43 (SEQ ID NO:512), DOM14-44 (SEQ ID NO:513), DOM14-45 (SEQ ID NO:514), DOM14-46 (SEQ ID NO:515), DOM14-47 (SEQ ID NO:516), DOM14-48 (SEQ ID NO:517), DOM14-49 (SEQ ID NO:518), DOM14-50 (SEQ ID NO:519), DOM14-51 (SEQ ID NO:520), DOM14-52 (SEQ ID NO:521), DOM14-53 (SEQ ID NO:522), DOM14-54 (SEQ ID NO:523), DOM14-55 (SEQ ID NO:524), DOM14-56 (SEQ ID NO:525), DOM14-57 (SEQ ID NO:526), DOM14-58 (SEQ ID NO:527), DOM14-59 (SEQ ID NO:528), DOM14-60 (SEQ ID NO:529), DOM14-61 (SEQ ID NO:530), DOM14-62 (SEQ ID NO:531), DOM14-63 (SEQ ID NO:532), DOM14-64 (SEQ ID NO:533), DOM14-65 (SEQ ID NO:534), DOM14-66 (SEQ ID NO:535), DOM14-67 (SEQ ID NO:536), DOM14-70 (SEQ ID NO:539), DOM14-68 (SEQ ID NO:537), and DOM14-69 (SEQ ID NO:538).

26. The ligand of claim 25, wherein said first immunoglobulin single variable domain or said second immunoglobulin single variable domain comprises an amino acid sequence that has at least about 90% amino acid sequence similarity with the amino acid sequence of a dAb selected from the group consisting of: DOM 14-1 (SEQ ID NO:477), DOM14-10 (SEQ ID NO:481), DOM14-100 (SEQ ID NO:540), DOM14-11 (SEQ ID NO:482), DOM14-12 (SEQ ID NO:483), DOM14-13 (SEQ ID NO:484), DOM14-14 (SEQ ID NO:485), DOM14-15 (SEQ ID NO:486), DOM14-16 (SEQ ID NO:487), DOM14-17 (SEQ ID NO:488), DOM14-18 (SEQ ID NO:489), DOM14-19 (SEQ ID NO:490), DOM14-2 (SEQ ID NO:478), DOM14-20 (SEQ ID NO:491), DOM14-21 (SEQ ID NO:492), DOM14-22 (SEQ ID NO:493), DOM14-23 (SEQ ID NO:494), DOM14-24 (SEQ ID NO:495), DOM14-25 (SEQ ID NO:496), DOM14-26 (SEQ ID NO:497), DOM14-27 (SEQ ID NO:498), DOM14-28 (SEQ ID NO:499), DOM14-3 (SEQ ID NO:479), DOM14-31 (SEQ ID NO:500), DOM14-32 (SEQ ID NO:501), DOM14-33 (SEQ ID NO:502), DOM14-34 (SEQ ID NO:503), DOM14-35 (SEQ ID NO:504), DOM14-36 (SEQ ID NO:505), DOM14-37 (SEQ ID NO:506), DOM14-38 (SEQ ID NO:507), DOM14-39 (SEQ ID NO:508), DOM14-4 (SEQ ID NO:480), DOM14-40 (SEQ ID NO:509), DOM14-41 (SEQ ID NO:510), DOM14-42 (SEQ ID NO:511), DOM14-43 (SEQ ID NO:512), DOM14-44 (SEQ ID NO:513), DOM14-45 (SEQ ID NO:514), DOM14-46 (SEQ ID NO:515), DOM14-47 (SEQ ID NO:516), DOM14-48 (SEQ ID NO:517), DOM14-49 (SEQ ID NO:518), DOM14-50 (SEQ ID NO:519), DOM14-51 (SEQ ID NO:520), DOM14-52 (SEQ ID NO:521), DOM14-53 (SEQ ID NO:522), DOM14-54 (SEQ ID NO:523), DOM14-55 (SEQ ID NO:524), DOM14-56 (SEQ ID NO:525), DOM14-57 (SEQ ID NO:526), DOM14-58 (SEQ ID NO:527), DOM14-59 (SEQ ID NO:528), DOM14-60 (SEQ ID NO:529), DOM14-61 (SEQ ID NO:530), DOM14-62 (SEQ ID NO:531), DOM14-63 (SEQ ID NO:532), DOM14-64 (SEQ ID NO:533), DOM14-65 (SEQ ID NO:534), DOM14-66 (SEQ ID NO:535), DOM14-67 (SEQ ID NO:536), DOM14-70 (SEQ ID NO:539), DOM14-68 (SEQ ID NO:537), and DOM14-69 (SEQ ID NO:538).

27. The ligand of claim 8, wherein first immunoglobulin single variable domain has a binding site with binding specificity CD38; and said second immunoglobulin single variable domain has a binding site with binding specificity for a cell surface target selected from the group consisting of CD138, CEA, CD56, VEGF, EGFR, and HER2.

28. The ligand of claim 27, wherein said second immunoglobulin single variable domain has a binding site with binding specificity for CD138.

29. The ligand of claim 8, wherein first immunoglobulin single variable domain has a binding site with binding specificity CD138; and said second immunoglobulin single variable domain has a binding site with binding specificity for a cell surface target selected from the group consisting of CD38, CEA, CD56, VEGF, EGFR, and HER2.

30. The ligand of claim 29, wherein said second immunoglobulin single variable domain has a binding site with binding specificity for CEA.

31. The ligand of claim 8, wherein first immunoglobulin single variable domain has a binding site with binding specificity CEA; and said second immunoglobulin single variable domain has a binding site with binding specificity for a cell surface target selected from the group consisting of CD38, CD38, CEA, VEGF, EGFR, and HER2.

32. The ligand of claim 31, wherein said second immunoglobulin single variable domain has a binding site with binding specificity for CD56.

33. The ligand of claim 1, wherein said ligand further comprises a toxin.

34. The ligand of claim 33, wherein said toxin is a surface active toxin.

35. The ligand of claim 34, wherein said surface active toxin comprises a free radical generator or a radionuclide.

36. The ligand of claim 35, wherein said toxin is a cytotoxin, surface active toxin, free radical generator, antimetabolite, protein, polypeptide, peptide, photoactive agent, antisense compound, chemotherapeutic, radionuclide or intrabodies.

37. The ligand of claim 1, wherein said ligand further comprises a half-life extending moiety.

38. The ligand of claim 37, wherein said half-life extending moiety is a polyalkylene glycol moiety, serum albumin or a fragment thereof, transferrin receptor or a transferrin-binding portion thereof, or an antibody or antibody fragment comprising a binding site for a polypeptide that enhances half-life in vivo.

39. The ligand of claim 38, wherein said half-life extending moiety is a polyethylene glycol moiety.

40. The ligand of claim 39, wherein said half-life extending moiety is an antibody or antibody fragment comprising a binding site for serum albumin or neonatal Fc receptor.

41. The ligand of claim 38, wherein said antibody or antibody fragment is an antibody fragment, and said antibody fragment is an immunoglobulin single variable domain.

42. The ligand of claim 41, wherein said immunoglobulin single variable domain competes for binding to human serum albumin with a dAb selected from the group consisting of: DOM7m-16 (SEQ ID NO: 541), DOM7m-12 (SEQ ID NO: 542), DOM7m-26 (SEQ ID NO: 543), DOM7r-1 (SEQ ID NO: 544), DOM7r-3 (SEQ ID NO: 545), DOM7r-4 (SEQ ID NO: 546), DOM7r-5 (SEQ ID NO: 547), DOM7r-7 (SEQ ID NO: 548), and DOM7r-8 (SEQ ID NO: 549), DOM7h-2 (SEQ ID NO: 550), DOM7h-3 (SEQ ID NO: 551), DOM7h-4 (SEQ ID NO: 552), DOM7h-6 (SEQ ID NO: 553), DOM7h-1 (SEQ ID NO: 555), DOM7h-7 (SEQ ID NO: 477), DOM7h-8 (SEQ ID NO: 564), DOM7r-13 (SEQ ID NO: 565), and DOM7r-14 (SEQ ID NO: 566), DOM7h-22 (SEQ ID NO: 557), DOM7h-23 (SEQ ID NO: 558), DOM7h-24 (SEQ ID NO: 559), DOM7h-25 (SEQ ID NO: 560), DOM7h-26 (SEQ ID NO: 561), DOM7h-21 (SEQ ID NO: 562), DOM7h-27 (SEQ ID NO: 563), DOM7r-15 (SEQ ID NO: 567), DOM7r-16 (SEQ ID NO: 568), DOM7r-17 (SEQ ID NO: 569), DOM7r-18 (SEQ ID NO: 570), DOM7r-19 (SEQ ID NO: 571), DOM7r-20 (SEQ ID NO: 572), DOM7r-21 (SEQ ID NO: 573), DOM7r-22 (SEQ ID NO: 574), DOM7r-23 (SEQ ID NO: 575), DOM7r-24 (SEQ ID NO: 576), DOM7r-25 (SEQ ID NO: 577), DOM7r-26 (SEQ ID NO: 578), DOM7r-27 (SEQ ID NO: 579), DOM7r-28 (SEQ ID NO: 580), DOM7r-29 (SEQ ID NO: 581), DOM7r-30 (SEQ ID NO: 582), DOM7r-31 (SEQ ID NO: 583), DOM7r-32 (SEQ ID NO: 584), and DOM7r-33 (SEQ ID NO: 585).

43. The ligand of claim 42, wherein said immunoglobulin single variable domain binds human serum albumin comprises an amino acid sequence that has at least 90% amino acid sequence identity with the amino acid sequence of a dAb selected from the group consisting of: DOM7m-16 (SEQ ID NO: 541), DOM7m-12 (SEQ ID NO: 542), DOM7m-26 (SEQ ID NO: 543), DOM7r-1 (SEQ ID NO: 544), DOM7r-3 (SEQ ID NO: 545), DOM7r-4 (SEQ ID NO: 546), DOM7r-5 (SEQ ID NO: 547), DOM7r-7 (SEQ ID NO: 548), and DOM7r-8 (SEQ ID NO: 549), DOM7h-2 (SEQ ID NO: 550), DOM7h-3 (SEQ ID NO: 551), DOM7h-4 (SEQ ID NO: 552), DOM7h-6 (SEQ ID NO: 553), DOM7h-1 (SEQ ID NO: 555), DOM7h-7 (SEQ ID NO: 477), DOM7h-8 (SEQ ID NO: 564), DOM7r-13 (SEQ ID NO: 565), and DOM7r-14 (SEQ ID NO: 566), DOM7h-22 (SEQ ID NO: 557), DOM7h-23 (SEQ ID NO: 558), DOM7h-24 (SEQ ID NO: 559), DOM7h-25 (SEQ ID NO: 560), DOM7h-26 (SEQ ID NO: 561), DOM7h-21 (SEQ ID NO: 562), DOM7h-27 (SEQ ID NO: 563), DOM7r-15 (SEQ ID NO: 567), DOM7r-16 (SEQ ID NO: 568), DOM7r-17 (SEQ ID NO: 569), DOM7r-18 (SEQ ID NO: 570), DOM7r-19 (SEQ ID NO: 571), DOM7r-20 (SEQ ID NO: 572), DOM7r-21 (SEQ ID NO: 573), DOM7r-22 (SEQ ID NO: 574), DOM7r-23 (SEQ ID NO: 575), DOM7r-24 (SEQ ID NO: 576), DOM7r-25 (SEQ ID NO: 577), DOM7r-26 (SEQ ID NO: 578), DOM7r-27 (SEQ ID NO: 579), DOM7r-28 (SEQ ID NO: 580), DOM7r-29 (SEQ ID NO: 581), DOM7r-30 (SEQ ID NO: 582), DOM7r-31 (SEQ ID NO: 583), DOM7r-32 (SEQ ID NO: 584), and DOM7r-33 (SEQ ID NO: 585).

44. A ligand comprising a first polypeptide domain having a binding site with binding specificity for a first cell surface target, a second polypeptide domain having a binding site with binding specificity for a second cell surface target, and at least one toxin moiety; wherein said first cell surface target and said second cell surface target are different, and said first cell surface target and said second cell surface target are present on a pathogenic cell; wherein said ligand binds said first cell surface target and said second cell surface target on said pathogenic cell with an avidity between about 10⁻⁶ M and about 10⁻¹² M; and wherein said ligand is internalized by said pathogenic cell.

45. The ligand of claim 44, wherein said ligand is preferentially internalized by said pathogenic cell.

46. The ligand of claim 44, wherein said ligand is not substantially internalized by single positive or normal cells.

47. The ligand of claim 44, wherein said ligand selectively binds said pathogenic cell.

48. The ligand of claim 44, wherein said toxin moiety comprises is a cytotoxin, surface active toxin, free radical generator, antimetabolite, protein, polypeptide, peptide, photoactive agent, antisense compound, chemotherapeutic, radionuclide or intrabodies.

49. The ligand of claim 44, wherein said toxin moiety comprises a surface active toxin.

50. The ligand of claim 49, wherein said surface active toxin comprises a free radical generator or a radionuclide.

51. The ligand of claim 44, wherein said first polypeptide domain binds said first cell surface target with low affinity and said second polypeptide domain binds said second cell surface target with low affinity.

52. The ligand of claim 51, wherein said first polypeptide domain and said second polypeptide domain each bind their respective cell surface targets with an affinity (KD) that is between about 10 μM and about 10 nM, as determined by surface plasmon resonance.

53. The ligand of claim 47, wherein said ligand selectively binds said pathogenic cell when said ligand is present at a concentration that is between about 1 pM and about 150 nM.

54. The ligand of claim 44, wherein the first polypeptide domain having a binding site with binding specificity for a first cell surface target and said second polypeptide domain having a binding site with binding specificity for a second cell surface target are a first immunoglobulin single variable domain, and a second immunoglobulin single variable domain, respectively.

55. The ligand of claim 54, wherein said first immunoglobulin single variable domain and/or said second immunoglobulin single variable domain is a V_{HH}.

56. The ligand of claim 54, wherein said first immunoglobulin single variable domain and said second immunoglobulin single variable domain are independently selected from the group consisting of a human V_{H}, and a human V_{L}.

57. The ligand of claim 54, wherein said first immunoglobulin single variable domain has a binding site with binding specificity for a cell surface target selected from the group consisting of CD38, CD138, carcinoembrionic antigen (CEA), CD56, vascular endothelial growth factor (VEGF), epidermal growth factor receptor (EGFR), and HER2.

58. The ligand of claim 57, wherein the second immunoglobulin single variable domain has a binding site with binding specificity for a cell surface target selected from the group consisting of CD38, CD138, CEA, CD56, VEGF, EGFR, and HER2, with the proviso that said first immunoglobulin single variable domain and said second immunoglobulin single variable domain do not bind the same cell surface target.

59. The ligand of claim 54, wherein said first immunoglobulin single variable domain or said second immunoglobulin single variable domain binds CD38 and competes for binding to CD38 with an anti-CD38 domain antibody (dAb) selected from the group consisting of: DOM11-14 (SEQ ID NO: 242). DOM11-22 (SEQ ID NO:246), DOM11-23 (SEQ ID NO:247), DOM11-25 (SEQ ID NO:249), DOM11-26 (SEQ ID NO:250), DOM11-27 (SEQ ID NO:251), DOM 11-29 (SEQ ID NO:253), DOM11-3A (SEQ ID NO:234), DOM11-30 (SEQ ID NO:254), DOM11-31 (SEQ ID NO:255), DOM11-32 (SEQ ID NO:256), DOM11-36 (SEQ ID NO:260), DOM11-4 (SEQ ID NO:235), DOM11-43 (SEQ ID NO:266), DOM11-44 (SEQ ID NO:267), DOM11-45 (SEQ ID NO:268), DOM11-5 (SEQ ID NO:236), DOM11-7 (SEQ ID NO:238), DOM11-1 (SEQ ID NO:232), DOM11-10 (SEQ ID NO:241), DOM11-16 (SEQ ID NO:243), DOM11-2 (SEQ ID NO:233), DOM11-20 (SEQ ID NO:244), DOM11-21 (SEQ ID NO:245), DOM11-24 (SEQ ID NO:248), DOM11-28 (SEQ ID NO:252), DOM11-33 (SEQ ID NO:257), DOM11-34 (SEQ ID NO:258), DOM11-35 (SEQ ID NO:259), DOM11-37 (SEQ ID NO:261), DOM11-38 (SEQ ID NO:262), DOM11-39 (SEQ ID NO:263), DOM11-41 (SEQ ID NO:264), DOM11-42 (SEQ ID NO:265), DOM11-6 (SEQ ID NO:237), DOM11-8 (SEQ ID NO:239), and DOM11-9 (SEQ ID NO:240).

60. The ligand of claim 54, wherein said first immunoglobulin single variable domain or said second immunoglobulin single variable domain binds CD38 and competes for binding to CD38 with an anti-CD38 domain antibody (dAb) selected from the group consisting of: DOM 11-3-1 (SEQ ID NO: 269), DOM 11-3-2 (SEQ ID NO: 270), DOM 11-3-3 (SEQ ID NO: 271), DOM 11-3-4 (SEQ ID NO: 272), DOM 11-3-6 (SEQ ID NO: 273), DOM 11-3-9 (SEQ ID NO: 274), DOM 11-3-10 (SEQ ID NO: 275), DOM 11-3-11 (SEQ ID NO: 276), DOM 11-3-14 (SEQ ID NO: 277), DOM 11-3-15 (SEQ ID NO: 278), DOM 11-3-17 (SEQ ID NO: 279), DOM 11-3-19 (SEQ ID NO: 280), DOM 11-3-20 (SEQ ID NO: 281), DOM 11-3-21 (SEQ ID NO: 282), DOM 11-3-22 (SEQ ID NO: 283), DOM 11-3-23 (SEQ ID NO: 284), DOM 11-3-24 (SEQ ID NO: 285), DOM 11-3-25 (SEQ ID NO: 286), DOM 11-3-26 (SEQ ID NO: 287), DOM 11-3-27 (SEQ ID NO: 288), DOM 11-3-28 (SEQ ID NO: 289), DOM 11-30-1 (SEQ ID NO: 290), DOM 11-30-2 (SEQ ID NO: 291), DOM 11-30-3 (SEQ ID NO: 292), DOM 11-30-5 (SEQ ID NO: 293), DOM 11-30-6 (SEQ ID NO: 294), DOM 11-30-7 (SEQ ID NO: 295), DOM 11-30-8 (SEQ ID NO: 296), DOM 11-30-9 (SEQ ID NO: 297), DOM 11-30-10 (SEQ ID NO: 298), DOM 11-30-11 (SEQ ID NO: 299), DOM 11-30-12 (SEQ ID NO: 300), DOM 11-30-13 (SEQ ID NO: 301), DOM 11-30-14 (SEQ ID NO: 302), DOM 11-30-15 (SEQ ID NO: 303), DOM 11-30-16 (SEQ ID NO: 304), and DOM 11-30-17 (SEQ ID NO: 305).

61. The ligand of claim 59, wherein said first immunoglobulin single variable domain or said second immunoglobulin single variable domain comprises an amino acid sequence that has at least about 90% amino acid sequence similarity with the amino acid sequence of a dAb selected from the group consisting of: DOM11-14 (SEQ ID NO: 242), DOM11-22 (SEQ ID NO:246), DOM11-23 (SEQ ID NO:247), DOM11-25 (SEQ ID NO:249), DOM11-26 (SEQ ID NO:250), DOM11-27 (SEQ ID NO:251), DOM 11-29 (SEQ ID NO:253), DOM11-3A (SEQ ID NO:234), DOM11-30 (SEQ ID NO:254), DOM11-31 (SEQ ID NO:255), DOM11-32 (SEQ ID NO:256) DOM11-36 (SEQ ID NO:260) DOM11-4 (SEQ ID NO:235), DOM11-43 (SEQ ID NO:266), DOM11-44 (SEQ ID NO:267), DOM11-45 (SEQ ID NO:268), DOM11-5 (SEQ ID NO:236), DOM11-7 (SEQ ID NO:238), DOM11-1 (SEQ ID NO:232), DOM11-10 (SEQ ID NO:241), DOM11-16 (SEQ ID NO:243), DOM11-2 (SEQ ID NO:233), DOM11-20 (SEQ ID NO:244), DOM11-21 (SEQ ID NO:245), DOM11-24 (SEQ ID NO:248), DOM11-28 (SEQ ID NO:252), DOM11-33 (SEQ ID NO:257), DOM11-34 (SEQ ID NO:258), DOM11-35 (SEQ ID NO:259) DOM11-37 (SEQ ID NO:261), DOM11-38 (SEQ ID NO:262) DOM111-39 (SEQ ID NO:263), DOM11-41 (SEQ ID NO:264), DOM11-42 (SEQ ID NO:265), DOM11-6 (SEQ ID NO:237), DOM11-8 (SEQ ID NO:239), and DOM11-9 (SEQ ID NO:240).

62. The ligand of claim 59, wherein said first immunoglobulin single variable domain or said second immunoglobulin single variable domain comprises an amino acid sequence that has at least about 90% amino acid sequence similarity with the amino acid sequence of a dAb selected from the group consisting of: DOM 11-3-1 (SEQ ID NO: 269), DOM 11-3-2 (SEQ ID NO: 270), DOM 11-3-3 (SEQ ID NO: 271), DOM 11-3-4 (SEQ ID NO: 272), DOM 11-3-6 (SEQ ID NO: 273), DOM 11-3-9 (SEQ ID NO: 274), DOM 11-3-10 (SEQ ID NO: 275), DOM 11-3-11 (SEQ ID NO: 276), DOM 11-3-14 (SEQ ID NO: 277), DOM 11-3-15 (SEQ ID NO: 278), DOM 11-3-17 (SEQ ID NO: 279), DOM 11-3-19 (SEQ ID NO: 280), DOM 11-3-20 (SEQ ID NO: 281), DOM 11-3-21 (SEQ ID NO: 282), DOM 11-3-22 (SEQ ID NO: 283), DOM 11-3-23 (SEQ ID NO: 284), DOM 11-3-24 (SEQ ID NO: 285), DOM 11-3-25 (SEQ ID NO: 286), DOM 11-3-26 (SEQ ID NO: 287), DOM 11-3-27 (SEQ ID NO: 288), DOM 11-3-28 (SEQ ID NO: 289), DOM 11-30-1 (SEQ ID NO: 290), DOM 11-30-2 (SEQ ID NO: 291), DOM 11-30-3 (SEQ ID NO: 292), DOM 11-30-5 (SEQ ID NO: 293), DOM 11-30-6 (SEQ ID NO: 294), DOM 11-30-7 (SEQ ID NO: 295), DOM 11-30-8 (SEQ ID NO: 296), DOM 11-30-9 (SEQ ID NO: 297), DOM 11-30-10 (SEQ ID NO: 298), DOM 11-30-11 (SEQ ID NO: 299), DOM 11-30-12 (SEQ ID NO: 300), DOM 11-30-13 (SEQ ID NO: 301), DOM 11-30-14 (SEQ ID NO: 302), DOM 11-30-15 (SEQ ID NO: 303), DOM 11-30-16 (SEQ ID NO: 304), and DOM 11-30-17 (SEQ ID NO: 305).

63. The ligand of claim 54, wherein said first immunoglobulin single variable domain or said second immunoglobulin single variable domain binds CD138 and competes for binding to CD138 with an anti-CD138 domain antibody (dAb) selected from the group consisting of: DOM12-1 (SEQ ID NO:306), DOM12-15 (SEQ ID NO:317), DOM12-17 (SEQ ID NO:318), DOM12-19 (SEQ ID NO:320), DOM12-2 (SEQ ID NO:307), DOM12-20 (SEQ ID NO:321), DOM12-21 (SEQ ID NO:322), DOM12-22 (SEQ ID NO:323), DOM12-3 (SEQ ID NO:308), DOM12-33 (SEQ ID NO:334), DOM12-39 (SEQ ID NO:340), DOM12-4 (SEQ ID NO:309), DOM12-40 (SEQ ID NO:341), DOM12-41 (SEQ ID NO:342), DOM12-42 (SEQ ID NO:343), DOM12-44 (SEQ ID NO:345), DOM12-46 (SEQ ID NO:347), DOM12-6 (SEQ ID NO:311) DOM12-7 (SEQ ID NO:312), DOM12-10 (SEQ ID NO:315), DOM12-11 (SEQ ID NO:316), DOM12-18 (SEQ ID NO:319), DOM12-23 (SEQ ID NO:324), DOM12-24 (SEQ ID NO:325), DOM12-25 (SEQ ID NO:326), DOM12-26 (SEQ ID NO:327), DOM12-27 (SEQ ID NO:328), DOM12-28 (SEQ ID NO:329), DOM12-29 (SEQ ID NO:330), DOM12-30 (SEQ ID NO:331), DOM12-31 (SEQ ID NO:332), DOM12-32 (SEQ ID NO:333), DOM12-34 (SEQ ID NO:335), DOM12-35 (SEQ ID NO:336), DOM12-36 (SEQ ID NO:337), DOM12-37 (SEQ ID NO:338), DOM12-38 (SEQ ID NO:339), DOM12-43 (SEQ ID NO:344), DOM12-45 (SEQ ID NO:346), DOM12-5 (SEQ ID NO:310), DOM12-8 (SEQ ID NO:313), and DOM12-9 (SEQ ID NO:314).

64. The ligand of claim 54, wherein said first immunoglobulin single variable domain or said second immunoglobulin single variable domain binds CD138 and competes for binding to CD138 with an anti-CD138 domain antibody (dAb) selected from the group consisting of: DOM 12-45-1 (SEQ ID NO: 348), DOM 12-45-2 (SEQ ID NO: 349), DOM 12-45-3 (SEQ ID NO: 350), DOM 12-45-4 (SEQ ID NO: 351), DOM 12-45-5 (SEQ ID NO: 352), DOM 12-45-6 (SEQ ID NO: 353), DOM 12-45-8 (SEQ ID NO: 354), DOM 12-45-9 (SEQ ID NO: 355), DOM 12-45-10 (SEQ ID NO: 356), DOM 12-45-11 (SEQ ID NO: 357), DOM 12-45-12 (SEQ ID NO: 358), DOM 12-45-13 (SEQ ID NO: 359), DOM 12-45-14 (SEQ ID NO: 360), DOM 12-45-15 (SEQ ID NO: 361), DOM 12-45-16 (SEQ ID NO: 362), DOM 12-45-17 (SEQ ID NO: 363), DOM 12-45-18 (SEQ ID NO: 364), DOM 12-45-19 (SEQ ID NO: 365), DOM 12-45-20 (SEQ ID NO: 366), DOM 12-45-21 (SEQ ID NO: 367), DOM 12-45-22 (SEQ ID NO: 368), DOM 12-45-23 (SEQ ID NO: 369), DOM 12-45-24 (SEQ ID NO: 370), DOM 12-45-25 (SEQ ID NO: 371), DOM 12-45-26 (SEQ ID NO: 372), DOM 12-45-27 (SEQ ID NO: 373), DOM 12-45-28 (SEQ ID NO: 374), DOM 12-45-29 (SEQ ID NO: 375), DOM 12-45-30 (SEQ ID NO: 376), DOM 12-45-31 (SEQ ID NO: 377), DOM 12-45-32 (SEQ ID NO: 378), DOM 12-45-33 (SEQ ID NO: 379), DOM 12-45-34 (SEQ ID NO: 380), DOM 12-45-35 (SEQ ID NO: 381), DOM 12-45-36 (SEQ ID NO: 382), DOM 12-45-37 (SEQ ID NO: 383), and DOM 12-45-38 (SEQ ID NO: 384).

65. The ligand of claim 63, wherein said first immunoglobulin single variable domain or said second immunoglobulin single variable domain comprises an amino acid sequence that has at least about 90% amino acid sequence similarity with the amino acid sequence of a dAb selected from the group consisting of: DOM12-1 (SEQ ID NO:306), DOM12-15 (SEQ ID NO:317), DOM12-17 (SEQ ID NO:318), DOM12-19 (SEQ ID NO:320), DOM12-2 (SEQ ID NO:307), DOM12-20 (SEQ ID NO:321), DOM12-21 (SEQ ID NO:322), DOM12-22 (SEQ ID NO:323), DOM12-3 (SEQ ID NO:308), DOM12-33 (SEQ ID NO:334), DOM12-39 (SEQ ID NO:340), DOM12-4 (SEQ ID NO:309), DOM12-40 (SEQ ID NO:341) DOM12-41 (SEQ ID NO:342), DOM12-42 (SEQ ID NO:343), DOM12-44 (SEQ ID NO:345) DOM12-46 (SEQ ID NO:347), DOM12-6 (SEQ ID NO:311), DOM 12-7 (SEQ ID NO:312), DOM 12-10 (SEQ ID NO:315), DOM 12-11 (SEQ ID NO:316), DOM12-18 (SEQ ID NO:319), DOM12-23 (SEQ ID NO:324), DOM12-24 (SEQ ID NO:325), DOM12-25 (SEQ ID NO:326), DOM12-26 (SEQ ID NO:327), DOM12-27 (SEQ ID NO:328), DOM12-28 (SEQ ID NO:329), DOM12-29 (SEQ ID NO:330), DOM12-30 (SEQ ID NO:331), DOM12-31 (SEQ ID NO:332), DOM12-32 (SEQ ID NO:333), DOM12-34 (SEQ ID NO:335). DOM12-35 (SEQ ID NO:336), DOM12-36 (SEQ ID NO:337), DOM12-37 (SEQ ID NO:338), DOM12-38 (SEQ ID NO:339), DOM12-43 (SEQ ID NO:344), DOM12-45 (SEQ ID NO:346), DOM12-5 (SEQ ID NO:310), DOM12-8 (SEQ ID NO:313). and DOM12-9 (SEQ ID NO:314).

66. The ligand of claim 63, wherein said first immunoglobulin single variable domain or said second immunoglobulin single variable domain comprises an amino acid sequence that has at least about 90% amino acid sequence similarity with the amino acid sequence of a dAb selected from the group consisting of: DOM 12-45-1 (SEQ ID NO: 348), DOM 12-45-2 (SEQ ID NO: 349), DOM 12-45-3 (SEQ ID NO: 350), DOM 12-45-4 (SEQ ID NO: 351), DOM 12-45-5 (SEQ ID NO: 352), DOM 12-45-6 (SEQ ID NO: 353), DOM 12-45-8 (SEQ ID NO: 354), DOM 12-45-9 (SEQ ID NO: 355), DOM 12-45-10 (SEQ ID NO: 356), DOM 12-45-11 (SEQ ID NO: 357), DOM 12-45-12 (SEQ ID NO: 358), DOM 12-45-13 (SEQ ID NO: 359), DOM 12-45-14 (SEQ ID NO: 360), DOM 12-45-15 (SEQ ID NO: 361), DOM 12-45-16 (SEQ ID NO: 362), DOM 12-45-17 (SEQ ID NO: 363), DOM 12-45-18 (SEQ ID NO: 364), DOM 12-45-19 (SEQ ID NO: 365), DOM 12-45-20 (SEQ ID NO: 366), DOM 12-45-21 (SEQ ID NO: 367), DOM 12-45-22 (SEQ ID NO: 368), DOM 12-45-23 (SEQ ID NO: 369), DOM 12-45-24 (SEQ ID NO: 370), DOM 12-45-25 (SEQ ID NO: 371), DOM 12-45-26 (SEQ ID NO: 372), DOM 12-45-27 (SEQ ID NO: 373), DOM 12-45-28 (SEQ ID NO: 374), DOM 12-45-29 (SEQ ID NO: 375), DOM 12-45-30 (SEQ ID NO: 376), DOM 12-45-31 (SEQ ID NO: 377), DOM 12-45-32 (SEQ ID NO: 378), DOM 12-45-33 (SEQ ID NO: 379), DOM 12-45-34 (SEQ ID NO: 380), DOM 12-45-35 (SEQ ID NO: 381), DOM 12-45-36 (SEQ ID NO: 382), DOM 12-45-37 (SEQ ID NO: 383), and DOM 12-45-38 (SEQ ID NO: 384).

67. The ligand of claim 54, wherein said first immunoglobulin single variable domain or said second immunoglobulin single variable domain binds CEA and competes for binding to CEA with an anti-CEA domain antibody (dAb) selected from the group consisting of: DOM13-1 (SEQ ID NO:385), DOM13-12 (SEQ ID NO:393), DOM13-13 (SEQ ID NO:394), DOM13-14 (SEQ ID NO:395), DOM13-15 (SEQ ID NO:396), DOM13-16 (SEQ ID NO:397), DOM13-17 (SEQ ID NO:398), DOM13-18 (SEQ ID NO:399), DOM13-19 (SEQ ID NO:400), DOM13-2 (SEQ ID NO:386), DOM13-20 (SEQ ID NO:401), DOM13-21 (SEQ ID NO:402), DOM13-22 (SEQ ID NO:403), DOM13-23 (SEQ ID NO:404), DOM13-24 (SEQ ID NO:405), DOM13-25 (SEQ ID NO:406), DOM13-26 (SEQ ID NO:407), DOM13-27 (SEQ ID NO:408), DOM13-28 (SEQ ID NO:409), DOM13-29 (SEQ ID NO:410), DOM13-3 (SEQ ID NO:387), DOM13-30 (SEQ ID NO:411), DOM13-31 (SEQ ID NO:412), DOM13-32 (SEQ ID NO:413), DOM13-33 (SEQ ID NO:414), DOM-13-34 (SEQ ID NO:415), DOM13-35 (SEQ ID NO:416), DOM13-36 (SEQ ID NO:417), DOM13-37 (SEQ ID NO:418), DOM13-4 (SEQ ID NO:388), DOM13-42 (SEQ ID NO:419), DOM13-43 (SEQ ID NO:420), DOM13-44 (SEQ ID NO:421), DOM13-45 (SEQ ID NO:422), DOM13-46 (SEQ ID NO:423), DOM13-47 (SEQ ID NO:424), DOM13-48 (SEQ ID NO:425), DOM13-49 (SEQ ID NO:426), DOM13-5 (SEQ ID NO:389), DOM13-50 (SEQ ID NO:427), DOM13-51 (SEQ ID NO:428), DOM13-52 (SEQ ID NO:429), DOM13-53 (SEQ ID NO:430), DOM13-54 (SEQ ID NO:431), DOM13-55 (SEQ ID NO:432), DOM13-56 (SEQ ID NO:433), DOM13-57 (SEQ ID NO:434), DOM13-58 (SEQ ID NO:435), DOM13-59 (SEQ ID NO:436), DOM13-6 (SEQ ID NO:390), DOM13-60 (SEQ ID NO:437), DOM13-61 (SEQ ID NO:438). DOM13-62 (SEQ ID NO:439), DOM13-63 (SEQ ID NO:440), DOM13-64 (SEQ ID NO:441), DOM13-65 (SEQ ID NO:442), DOM13-66 (SEQ ID NO:443), DOM13-67 (SEQ ID NO:444), DOM13-68 (SEQ ID NO:445), DOM13-69 (SEQ ID NO:446), DOM13-7 (SEQ ID NO:391), DOM13-70 (SEQ ID NO:447), DOM13-71 (SEQ ID NO:448), DOM13-72 (SEQ ID NO:449), DOM13-73 (SEQ ID NO:450), DOM13-74 (SEQ ID NO:451), DOM13-75 (SEQ ID NO:452), DOM13-76 (SEQ ID NO:453), DOM13-77 (SEQ ID NO:454), DOM13-78 (SEQ ID NO:455), DOM13-79 (SEQ ID NO:456), DOM13-8 (SEQ ID NO:392), DOM13-80 (SEQ ID NO:457), DOM13-81 (SEQ ID NO:458), DOM13-82 (SEQ ID NO:459), DOM13-83 (SEQ ID NO:460), DOM13-84 (SEQ ID NO:461), DOM13-85 (SEQ ID NO:462), DOM13-86 (SEQ ID NO:463), DOM13-87 (SEQ ID NO:464), DOM13-88 (SEQ ID NO:465), DOM13-89 (SEQ ID NO:466), DOM13-90 (SEQ ID NO:467), DOM13-91 (SEQ ID NO:468), DOM13-92 (SEQ ID NO:469), DOM13-93 (SEQ ID NO:470), DOM13-94 (SEQ ID NO:471), and DOM13-95 (SEQ ID NO:472).

68. The ligand of claim 54, wherein said first immunoglobulin single variable domain or said second immunoglobulin single variable domain binds CEA and competes for binding to CEA with an anti-CEA domain antibody (dAb) selected from the group consisting of: DOM 13-25-3 (SEQ ID NO: 473), DOM 13-25-23 (SEQ ID NO: 474), DOM 13-25-27 (SEQ ID NO: 475), and DOM 13-25-80 (SEQ ID NO: 476).

69. The ligand of claim 67, wherein said first immunoglobulin single variable domain or said second immunoglobulin single variable domain comprises an amino acid sequence that has at least about 90% amino acid sequence similarity with the amino acid sequence of a dAb selected from the group consisting of: DOM13-1 (SEQ ID NO:385), DOM13-12 (SEQ ID NO:393), DOM13-13 (SEQ ID NO:394), DOM13-14 (SEQ ID NO:395), DOM13-15 (SEQ ID NO:396), DOM13-16 (SEQ ID NO:397), DOM13-17 (SEQ ID NO:398), DOM13-18 (SEQ ID NO:399), DOM13-19 (SEQ ID NO:400), DOM13-2 (SEQ ID NO:386). DOM13-20 (SEQ ID NO:401), DOM13-21 (SEQ ID NO:402), DOM13-22 (SEQ ID NO:403), DOM13-23 (SEQ ID NO:404), DOM13-24 (SEQ ID NO:405), DOM13-25 (SEQ ID NO:406), DOM13-26 (SEQ ID NO:407), DOM13-27 (SEQ ID NO:408), DOM13-28 (SEQ ID NO:409), DOM13-29 (SEQ ID NO:410), DOM13-3 (SEQ ID NO:387), DOM13-30 (SEQ ID NO:411). DOM13-31 (SEQ ID NO:412), DOM13-32 (SEQ ID NO:413), DOM13-33 (SEQ ID NO:414), DOM-13-34 (SEQ ID NO:415), DOM13-35 (SEQ ID NO:416), DOM13-36 (SEQ ID NO:417), DOM13-37 (SEQ ID NO:418), DOM13-4 (SEQ ID NO:388), DOM13-42 (SEQ ID NO:419), DOM13-43 (SEQ ID NO:420), DOM13-44 (SEQ ID NO:421), DOM13-45 (SEQ ID NO:422), DOM13-46 (SEQ ID NO:423), DOM13-47 (SEQ ID NO:424), DOM13-48 (SEQ ID NO:425), DOM13-49 (SEQ ID NO:426), DOM13-5 (SEQ ID NO:389), DOM13-50 (SEQ ID NO:427), DOM13-51 (SEQ ID NO:428), DOM13-52 (SEQ ID NO:429), DOM13-53 (SEQ ID NO:430), DOM13-54 (SEQ ID NO:431), DOM13-55 (SEQ ID NO:432), DOM13-56 (SEQ ID NO:433), DOM13-57 (SEQ ID NO:434), DOM13-58 (SEQ ID NO:435), DOM13-59 (SEQ ID NO:436), DOM13-6 (SEQ ID NO:390), DOM13-60 (SEQ ID NO:437), DOM13-61 (SEQ ID NO:438), DOM13-62 (SEQ ID NO:439), DOM13-63 (SEQ ID NO:440), DOM13-64 (SEQ ID NO:441), DOM13-65 (SEQ ID NO:442), DOM13-66 (SEQ ID NO:443), DOM13-67 (SEQ ID NO:444), DOM13-68 (SEQ ID NO:445), DOM13-69 (SEQ ID NO:446), DOM13-7 (SEQ ID NO:391), DOM13-70 (SEQ ID NO:447), DOM13-71 (SEQ ID NO:448), DOM13-72 (SEQ ID NO:449), DOM13-73 (SEQ ID NO:450), DOM13-74 (SEQ ID NO:451), DOM13-75 (SEQ ID NO:452), DOM13-76 (SEQ ID NO:453), DOM13-77 (SEQ ID NO:454), DOM13-78 (SEQ ID NO:455), DOM13-79 (SEQ ID NO:456), DOM13-8 (SEQ ID NO:392), DOM13-80 (SEQ ID NO:457), DOM13-81 (SEQ ID NO:458), DOM13-82 (SEQ ID NO:459), DOM13-83 (SEQ ID NO:460), DOM13-84 (SEQ ID NO:461), DOM13-85 (SEQ ID NO:462), DOM13-86 (SEQ ID NO:463), DOM13-87 (SEQ ID NO:464), DOM13-88 (SEQ ID NO:465), DOM13-89 (SEQ ID NO:466), DOM13-90 (SEQ ID NO:467), DOM13-91 (SEQ ID NO:468), DOM13-92 (SEQ ID NO:469), DOM13-93 (SEQ ID NO:470), DOM13-94 (SEQ ID NO:471), and DOM13-95 (SEQ ID NO:472).

70. The ligand of claim 67, wherein said first immunoglobulin single variable domain or said second immunoglobulin single variable domain comprises an amino acid sequence that has at least about 90% amino acid sequence similarity with the amino acid sequence of a dAb selected from the group consisting of: DOM 13-25-3 (SEQ ID NO: 473), DOM 13-25-23 (SEQ ID NO: 474), DOM 13-25-27 (SEQ ID NO: 475), and DOM 13-25-80 (SEQ ID NO: 476).

71. The ligand of claim 54, wherein said first immunoglobulin single variable domain or said second immunoglobulin single variable domain binds CD56 and competes for binding to CD56 with an anti-CD56 domain antibody (dAb) selected from the group consisting of: DOM14-1 (SEQ ID NO:477), DOM14-10 (SEQ ID NO:481), DOM14-100 (SEQ ID NO:540), DOM14-11 (SEQ ID NO:482), DOM14-12 (SEQ ID NO:483), DOM14-13 (SEQ ID NO:484), DOM14-14 (SEQ ID NO:485), DOM14-15 (SEQ ID NO:486), DOM14-16 (SEQ ID NO:487), DOM14-17 (SEQ ID NO:488), DOM14-18 (SEQ ID NO:489), DOM14-19 (SEQ ID NO:490), DOM14-2 (SEQ ID NO:478), DOM14-20 (SEQ ID NO:491), DOM14-21 (SEQ ID NO:492), DOM14-22 (SEQ ID NO:493), DOM14-23 (SEQ ID NO:494), DOM14-24 (SEQ ID NO:495), DOM14-25 (SEQ ID NO:496), DOM14-26 (SEQ ID NO:497), DOM14-27 (SEQ ID NO:498), DOM14-28 (SEQ ID NO:499), DOM14-3 (SEQ ID NO:479), DOM14-31 (SEQ ID NO:500), DOM14-32 (SEQ ID NO:501), DOM14-33 (SEQ ID NO:502), DOM14-34 (SEQ ID NO:503), DOM14-35 (SEQ ID NO:504), DOM14-36 (SEQ ID NO:505), DOM14-37 (SEQ ID NO:506), DOM14-38 (SEQ ID NO:507), DOM14-39 (SEQ ID NO:508), DOM14-4 (SEQ ID NO:480), DOM14-40 (SEQ ID NO:509), DOM14-41 (SEQ ID NO:510), DOM14-42 (SEQ ID NO:511), DOM14-43 (SEQ ID NO:512), DOM14-44 (SEQ ID NO:513), DOM14-45 (SEQ ID NO:514), DOM14-46 (SEQ ID NO:515), DOM14-47 (SEQ ID NO:516), DOM14-48 (SEQ ID NO:517), DOM14-49 (SEQ ID NO:518), DOM14-50 (SEQ ID NO:519), DOM14-51 (SEQ ID NO:520), DOM14-52 (SEQ ID NO:521), DOM14-53 (SEQ ID NO:522), DOM14-54 (SEQ ID NO:523), DOM14-55 (SEQ ID NO:524), DOM14-56 (SEQ ID NO:525), DOM14-57 (SEQ ID NO:526), DOM14-58 (SEQ ID NO:527), DOM14-59 (SEQ ID NO:528), DOM14-60 (SEQ ID NO:529), DOM14-61 (SEQ ID NO:530), DOM14-62 (SEQ ID NO:531), DOM14-63 (SEQ ID NO:532), DOM14-64 (SEQ ID NO:533), DOM14-65 (SEQ ID NO:534), DOM14-66 (SEQ ID NO:535), DOM14-67 (SEQ ID NO:536), DOM14-70 (SEQ ID NO:539), DOM14-68 (SEQ ID NO:537), and DOM14-69 (SEQ ID NO:538).

72. The ligand of claim 71, wherein said first immunoglobulin single variable domain or said second immunoglobulin single variable domain comprises an amino acid sequence that has at least about 90% amino acid sequence similarity with the amino acid sequence of a dAb selected from the group consisting of: DOM14-1 (SEQ ID NO:477), DOM14-10 (SEQ ID NO:481), DOM14-100 (SEQ ID NO:540), DOM14-11 (SEQ ID NO:482), DOM14-12 (SEQ ID NO:483), DOM14-13 (SEQ ID NO:484), DOM14-14 (SEQ ID NO:485), DOM14-15 (SEQ ID NO:486), DOM14-16 (SEQ ID NO:487), DOM14-17 (SEQ ID NO:488), DOM14-18 (SEQ ID NO:489), DOM14-19 (SEQ ID NO:490), DOM14-2 (SEQ ID NO:478), DOM14-20 (SEQ ID NO:491), DOM14-21 (SEQ ID NO:492), DOM14-22 (SEQ ID NO:493), DOM14-23 (SEQ ID NO:494), DOM14-24 (SEQ ID NO:495), DOM14-25 (SEQ ID NO:496), DOM14-26 (SEQ ID NO:497), DOM14-27 (SEQ ID NO:498), DOM14-28 (SEQ ID NO:499), DOM14-3 (SEQ ID NO:479), DOM14-31 (SEQ ID NO:500), DOM14-32 (SEQ ID NO:501), DOM14-33 (SEQ ID NO:502), DOM14-34 (SEQ ID NO:503), DOM14-35 (SEQ ID NO:504), DOM14-36 (SEQ ID NO:505), DOM14-37 (SEQ ID NO:506), DOM14-38 (SEQ ID NO:507), DOM14-39 (SEQ ID NO:508), DOM14-4 (SEQ ID NO:480), DOM14-40 (SEQ ID NO:509), DOM14-41 (SEQ ID NO:510), DOM14-42 (SEQ ID NO:511), DOM14-43 (SEQ ID NO:512), DOM14-44 (SEQ ID NO:513), DOM14-45 (SEQ ID NO:514), DOM14-46 (SEQ ID NO:515), DOM14-47 (SEQ ID NO:516), DOM14-48 (SEQ ID NO:517), DOM14-49 (SEQ ID NO:518), DOM14-50 (SEQ ID NO:519), DOM14-51 (SEQ ID NO:520), DOM14-52 (SEQ ID NO:521), DOM14-53 (SEQ ID NO:522), DOM14-54 (SEQ ID NO:523), DOM14-55 (SEQ ID NO:524), DOM14-56 (SEQ ID NO:525), DOM14-57 (SEQ ID NO:526), DOM14-58 (SEQ ID NO:527), DOM14-59 (SEQ ID NO:528), DOM14-60 (SEQ ID NO:529), DOM14-61 (SEQ ID NO:530), DOM14-62 (SEQ ID NO:531), DOM14-63 (SEQ ID NO:532), DOM14-64 (SEQ ID NO:533), DOM14-65 (SEQ ID NO:534), DOM14-66 (SEQ ID NO:535), DOM14-67 (SEQ ID NO:536), DOM14-70 (SEQ ID NO:539), DOM14-68 (SEQ ID NO:537), and DOM14-69 (SEQ ID NO:538).

73. The ligand of claim 54, wherein first immunoglobulin single variable domain has a binding site with binding specificity CD38; and said second immunoglobulin single variable domain has a binding site with binding specificity for a cell surface target selected from the group consisting of CD138, CEA, CD56, VEGF, EGFR, and HER2.

74. The ligand of claim 73, wherein said second immunoglobulin single variable domain has a binding site with binding specificity for CD138.

75. The ligand of claim 54, wherein first immunoglobulin single variable domain has a binding site with binding specificity CD138; and said second immunoglobulin single variable domain has a binding site with binding specificity for a cell surface target selected from the group consisting of CD38, CEA, CD56, VEGF, EGFR, and HER2.

76. The ligand of claim 75, wherein said second immunoglobulin single variable domain has a binding site with binding specificity for CEA.

77. The ligand of claim 54, wherein first immunoglobulin single variable domain has a binding site with binding specificity CEA; and said second immunoglobulin single variable domain has a binding site with binding specificity for a cell surface target selected from the group consisting of CD38, CD38 CD138, CEA, VEGF, EGFR, and HER2.

78. The ligand of claim 77, wherein said second immunoglobulin single variable domain has a binding site with binding specificity for CD56.

79. The ligand of claim 44, wherein said ligand further comprises a half-life extending moiety.

80. The ligand of claim 79, wherein said half-life extending moiety is a polyalkylene glycol moiety, serum albumin or a fragment thereof, transferrin receptor or a transferrin-binding portion thereof, or an antibody or antibody fragment comprising a binding site for a polypeptide that enhances half-life in vivo.

81. The ligand of claim 80, wherein said half-life extending moiety is a polyethylene glycol moiety.

82. The ligand of claim 80, wherein said half-life extending moiety is an antibody or antibody fragment comprising a binding site for serum albumin or neonatal Fc receptor.

83. The ligand of claim 80, wherein said antibody or antibody fragment is an antibody fragment, and said antibody fragment is an immunoglobulin single variable domain.

84. The ligand of claim 83, wherein said immunoglobulin single variable domain competes for binding to human serum albumin with a dAb selected from the group consisting of: DOM7m-16 (SEQ ID NO: 541), DOM7m-12 (SEQ ID NO: 542), DOM7m-26 (SEQ ID NO: 543), DOM7r-1 (SEQ ID NO: 544), DOM7r-3 (SEQ ID NO: 545), DOM7r-4 (SEQ ID NO: 546), DOM7r-5 (SEQ ID NO: 547), DOM7r-7 (SEQ ID NO: 548), and DOM7r-8 (SEQ ID NO: 549), DOM7h-2 (SEQ ID NO: 550), DOM7h-3 (SEQ ID NO: 551), DOM7h-4 (SEQ ID NO: 552), DOM7h-6 (SEQ ID NO: 553), DOM7h-1 (SEQ ID NO: 555), DOM7h-7 (SEQ ID NO: 477), DOM7h-8 (SEQ ID NO: 564), DOM7r-13 (SEQ ID NO: 565), and DOM7r-14 (SEQ ID NO: 566), DOM7h-22 (SEQ ID NO: 557), DOM7h-23 (SEQ ID NO: 558), DOM7h-24 (SEQ ID NO: 559), DOM7h-25 (SEQ ID NO: 560), DOM7h-26 (SEQ ID NO: 561), DOM7h-21 (SEQ ID NO: 562), DOM7h-27 (SEQ ID NO: 563), DOM7r-15 (SEQ ID NO: 567), DOM7r-16 (SEQ ID NO: 568), DOM7r-17 (SEQ ID NO: 569), DOM7r-18 (SEQ ID NO: 570), DOM7r-19 (SEQ ID NO: 571), DOM7r-20 (SEQ ID NO: 572), DOM7r-21 (SEQ ID NO: 573), DOM7r-22 (SEQ ID NO: 574), DOM7r-23 (SEQ ID NO: 575), DOM7r-24 (SEQ ID NO: 576), DOM7r-25 (SEQ ID NO: 577), DOM7r-26 (SEQ ID NO: 578), DOM7r-27 (SEQ ID NO: 579), DOM7r-28 (SEQ ID NO: 580), DOM7r-29 (SEQ ID NO: 581), DOM7r-30 (SEQ ID NO: 582), DOM7r-31 (SEQ ID NO: 583), DOM7r-32 (SEQ ID NO: 584), and DOM7r-33 (SEQ ID NO: 585).

85. The ligand of claim 84, wherein said immunoglobulin single variable domain binds human serum albumin comprises an amino acid sequence that has at least 90% amino acid sequence identity with the amino acid sequence of a dAb selected from the group consisting of: DOM7m-16 (SEQ ID NO: 541), DOM7m-12 (SEQ ID NO: 542), DOM7m-26 (SEQ ID NO: 543), DOM7r-1 (SEQ ID NO: 544), DOM7r-3 (SEQ ID NO: 545), DOM7r-4 (SEQ ID NO: 546), DOM7r-5 (SEQ ID NO: 547), DOM7r-7 (SEQ ID NO: 548), and DOM7r-8 (SEQ ID NO: 549), DOM7h-2 (SEQ ID NO: 550), DOM7h-3 (SEQ ID NO: 551), DOM7h-4 (SEQ ID NO: 552), DOM7h-6 (SEQ ID NO: 553), DOM7h-1 (SEQ ID NO: 555), DOM7h-7 (SEQ ID NO: 477), DOM7h-8 (SEQ ID NO: 564), DOM7r-13 (SEQ ID NO: 565), and DOM7r-14 (SEQ ID NO: 566), DOM7h-22 (SEQ ID NO: 557), DOM7h-23 (SEQ ID NO: 558), DOM7h-24 (SEQ ID NO: 559), DOM7h-25 (SEQ ID NO: 560), DOM7h-26 (SEQ ID NO: 561), DOM7h-21 (SEQ ID NO: 562), DOM7h-27 (SEQ ID NO: 563), DOM7r-15 (SEQ ID NO: 567), DOM7r-16 (SEQ ID NO: 568), DOM7r-17 (SEQ ID NO: 569), DOM7r-18 (SEQ ID NO: 570), DOM7r-19 (SEQ ID NO: 571), DOM7r-20 (SEQ ID NO: 572), DOM7r-21 (SEQ ID NO: 573), DOM7r-22 (SEQ ID NO: 574), DOM7r-23 (SEQ ID NO: 575), DOM7r-24 (SEQ ID NO: 576), DOM7r-25 (SEQ ID NO: 577), DOM7r-26 (SEQ ID NO: 578), DOM7r-27 (SEQ ID NO: 579), DOM7r-28 (SEQ ID NO: 580), DOM7r-29 (SEQ ID NO: 581), DOM7r-30 (SEQ ID NO: 582), DOM7r-31 (SEQ ID NO: 583), DOM7r-32 (SEQ ID NO: 584), and DOM7r-33 (SEQ ID NO: 585).

86.-88. (canceled)

89. A method of delivering a toxin internally to a cell, comprising contacting said cell with a ligand of any one of claim 1, wherein ligand is internalized and the toxin is delivered internally.

90. A method for treating cancer comprising administering to a subject in need thereof a therapeutically effective amount of ligand of claim 1.

91. The method of claim 90, wherein the cancer is multiple myeloma.

92. The method of claim 91, wherein the cancer is lung carcinoma.

93. A pharmaceutical composition comprising a ligand of claim 1 and a physiologically acceptable carrier.

94. The composition of claim 93, wherein said composition comprises a vehicle for intravenous, intramuscular, intraperitoneal, intraarterial, intrathecal, intraarticular, or subcutaneous administration.

95. The composition of claim 93, wherein said composition comprises a vehicle is for pulmonary, intranasal, vaginal, or rectal administration.

96. A drug delivery device comprising the composition of claim 93.

97. The drug delivery device of claim 96, wherein said drug delivery device is selected from the group consisting of a parenteral delivery device, intravenous delivery device, intramuscular delivery device, intraperitoneal delivery device, transdermal delivery device, pulmonary delivery device, intraarterial delivery device, intrathecal delivery device, intraarticular delivery device, subcutaneous delivery device, intranasal delivery device, vaginal delivery device, and rectal delivery device.

98. The drug delivery device of claim 96, wherein said device is selected from the group consisting of a syringe, a transdermal delivery device, a capsule, a tablet, a nebulizer, an inhaler, an atomizer, an aerosolizer, a mister, a dry powder inhaler, a metered dose inhaler, a metered dose sprayer, a metered dose mister, a metered dose atomizer, a catheter.

99.-102. (canceled)

103. An isolated or recombinant nucleic acid encoding a ligand of claim 1.

104. A vector comprising the recombinant nucleic acid of claim 103.

105. A host cell comprising the recombinant nucleic acid of claim 103.

106. A method for producing a ligand comprising maintaining the host cell of claim 105 under conditions suitable for expression of said nucleic acid or vector, whereby a ligand is produced.

107. The method of claim 106, further comprising isolating the ligand.

108. A method for treating cancer, comprising administering to a subject in need thereof a therapeutically effective amount of ligand of claim 1 and a chemotherapeutic agent, wherein the chemotherapeutic agent is administered at a low dose.

109. A method of selectively killing cancer cells over normal cells, in a subject in need thereof, comprising administering to said subject an affective amount of a ligand comprising a first polypeptide domain having a binding site with binding specificity for a first cell surface target, a second polypeptide domain having a binding site with binding specificity for a second cell surface target, and a toxin,
wherein said first cell surface target and said second cell surface target are different, and said first cell surface target and said second cell surface target are present on a cancer cell in an amount greater than a normal cell;
wherein said ligand binds said first cell surface target and said second cell surface target on said cancer cell; and
wherein said ligand is internalized by said cancer cell and is killed by the toxin.

110. A method for delivering a therapeutic agent intracellularly, comprising administering a ligand comprising a first polypeptide domain having a binding site with binding specificity for a first cell surface target and a second polypeptide domain having a binding site with binding specificity for a second cell surface target,
wherein said first cell surface target and said second cell surface target are different, and said first cell surface target and said second cell surface target are present on a pathogenic cell;
wherein said ligand binds said first cell surface target and said second cell surface target on said pathogenic cell; and
wherein said ligand is internalized by said pathogenic cell.

111. The method of claim 110, wherein the internalized ligand is delivered to a cathepsin B compartment in a cell.


SEQUENCE LISTING (711 sequences)

SEQ ID NO: 1 - DNA, 323 bp, Homo sapiens
gacatccagatgacccagtctccatcctccctgtctgcatctgtaggagaccgtgtcaccatcacttgccgggcaagtcagaatattgattctcgtttaagttggtaccagcagaaaccagggaaagcccctaagctcctgatctataggacgtccgttttgcaaagtggggtcccatcacgtttcagtggcagtggatctgggacagatttcactctcaccatcagcagtctgcaacctgaagattttgctacgtactactgtcaacagtgggatatgtttcctttgacgttcggccaagggaccaaggtggaagtcaaacg

SEQ ID NO: 2 - DNA, 324 bp, Homo sapiens
gacatccagataacccagtctccatcctccctgtctgcatctgtaggagaccgtatcaccatcacttgccgggcaagtcagaagattgagaatgatttagcttggtaccagcagaaaccagggaaagcccctaagctcctgatctattatacttccattttgcaaagtggggtcccatcacgtttcagtggcagtggatctgggacagatttcactctcaccatcagcagtctgcaacctgaagattttgctacgtactactgtcaacagcggaggtatgtgcctgcgacgttcggccaagggaccaaggtggaaatcaaacgg

SEQ ID NO: 3 - DNA, 323 bp, Homo sapiens
gacatccagatgacccagtctccatcctccctgtctgcatctgtaggagaccgtgttaccatcacttgccgggcaagtcagagtattaatgttcggttaatttggtaccagcagaaaccagggaaagaccctaagctcctgatctattcttcttcccatttgcaaagtggggtcccatcacgtttcagtggcagtggatctgggacagatttcactctcaccatcagcagtctgcaacctgaagattttgctacgtactactgtcaacagtatcattatacgccttttacgttcggccaagggaccaaggtggaaatcaaacg

SEQ ID NO: 4 - DNA, 323 bp, Homo sapiens
gacatccagatgacccagtctccatcctccctgtctgcatctgtaggagaccgtgtcaccatcacttgccgggcaagtcagtatattaatactcttttatcttggtaccagcagaaaccagggaaagcccctaagctcctgatctatgcgcagtcccgtttgcaaagtggggtcccatcacgtttcagtggcagtggatctgggacagatttcactctcaccatcagcagtctgcaacctgaagattttgctacgtactactgtcaacagtttgcttttcgtccttatacgttcggccaagggaccaaggtggaaatcaaacg

SEQ ID NO: 5 - DNA, 324 bp, Homo sapiens
gacatccaggtgacccagtctccatcctccctgtctgcatctgtaggagaccgtgtcaccatcacttgccgggcaagtcaggagatttcttcgtgtttaaattggtatcagcagaaaccagggaaagcccctaagctcctgatctattgtacgtccgtgttgcaaagtggggtcccatcacgtttcagtggcagtggatctgggacagatttcactctcaccatcagcagtctgcaacctgaagattttgctacgtactactgtcaacagaggtttgggaatcctctgacgttcggccaagggaccaaggtggaaatcaaacgg

SEQ ID NO: 6 - DNA, 324 bp, Homo sapiens
gacatccagatgacccagtctccatcctccctgtctgcatctgtaggagaccgtgtcaccatcacttgccgggcaagtcagccgattgatggtaatttaaggtggtaccagcagaaaccagggaaagcccctaggctcctgatctattttacttccattttgcaaagtggggtcccatcacgtttcagtggcagtggatctgggacagatttcactctcaccatcagcagtctgcaacctgaagattttgctacgtactactgtcaacagcataggcattggcctgcgacgttcggccaagggaccaaggtggaaatcaaacgg

SEQ ID NO: 7 - DNA, 324 bp, Homo sapiens
gacatccagatgacccagtctccatcctccctgtctgcatctgtaggagaccgtgtcaccatcacttgccgggcaagtcagtggattggtgattcgttagtttggtaccagcagaaaccagggaaagcccctaagctcctgatctattttggttccattttgcaaagtggggtcccatcacgtttcagtggcagtggatctgggacagatttcactctcaccatcagcagtctgcaacctgaagattttgctacgtactactgtcaacagcataagacgtcgcctagtacgttcggccaagggaccaaggtggaaatcaaacgg

SEQ ID NO: 8 - DNA, 324 bp, Homo sapiens
gacatccagatgacccagtctccatcctccctgtctgcatttgtaggagaccgtgtcaccatcacttgccgggcaagtcaggatattgagggtcagttacggtggtaccagcagaaaccagggaaagcccctaagctcctgatctattttggttccctgttgcaaagtggggtcccatcacgtttcagtggcagtggatctgggacggtttacactctcaccatcagcagtctgcaacctgaagattttgctacgtactactgtcaacagtcgcatctttttcctgcgacgttcggccaagggaccaaggtggaaatcaaacgg

SEQ ID NO: 9 - DNA, 324 bp, Homo sapiens
gacatccagatgacccagtctccatcctccctgtctgcatctgtaggagaccgtgtcaccatcacttgccgggcaagtcaggagattcatgattatttaagttggtaccagcagaaaccagggaaagcccctaagctcctgatctatctgtcttcccgtttgcaaagtggggtcccatcacgtttcagtggcagtggatctgggacagatttcactctcaccatcagcagtctgcaacctgaagattttgctacgtactactgtcaacagtatcataaggggccttacacgttcggccaagggaccaaggtggaaatcaaacgg

SEQ ID NO: 10 - DNA, 324 bp, Homo sapiens
gacatccagatgacccagtctccatcctccctgtctgcatctgtaggagaccgtgtcaccatcacttgccgggcaagtactgagattggtaggcgtttattgtggtaccagcagaaaccagggaaagcccctaagctcctgatctcggctggttccgtgttgcaaagtggggtcccatcacgtttcagtggcagtggatctgggacagatttcactctcaccatcagcagtctgcaacctgaagattttgctacgtactactgtggtcagtatcatgagcggcctgagacgttcggccaagggaccaaggtggaaatcaaacgg

SEQ ID NO: 11 - DNA, 323 bp, Homo sapiens
gacatccagatgacccagtctccatcctccctgtctgcatctgtaggagaccgtgtcaccatcacttgccgggcaagtcaggagattcatgattatttaagttggtaccagcagaaaccagggaaagcccctaagctcctgatctatctgtcttcccgtctgcaaagtggggtcccatcacgtttcagtggcagtagatctgggacagatttcactctcaccatcagcagtctgcaacctgaagattttgctacgtactactgtcaacagtatcataagtatccttatacgttcggccaagggaccaaggtggaaatcaaacg

SEQ ID NO: 12 - DNA, 324 bp, Homo sapiens
gacatccagatgacccagtctccatcctccctgtctgcatctgtaggagaccgtgtcaccatcacttgccgggcaagtcagtttattgggcggtatttatattggtatcagcagaaaccagggaaagcccctaagctcctgatctatgatacttccgcgttgcaaagtggggtcccatcacgtttcagtggcagtggatctgggacagatttcactctcaccatcagcagtctgcaacctgaagattttgctacgtactactgtcaacaggcttatctgggtcctgctacgttcggccaagggaccaaggtggaaatcaaacgg

SEQ ID NO: 13 - DNA, 324 bp, Homo sapiens
gacatccagatgacccagtctccatcctccctgtctgcatctgtaggagaccgtgtcaccatcacttgccgggcaagtcagagcattagccgctatttaaattggtaccagcagaaaccagggaaagcccctaagctcctgatctatatgatttcccggttgcaaagtggggtcccatcacgtttcagtggcagtggatctgggacagatttcactctcaccatcagcagtctgcaacccgaagattttgctacgtactactgtcaacagaattatctggcgcctgatacgttcggccaagggaccaaggtggaaatcaaacgg

SEQ ID NO: 14 - DNA, 324 bp, Homo sapiens
gacatccagatgacccagtctccatcctccctgtctgcatctgtaggagaccgtgtcaccatcacttgccgggcaagtcagaatattttgtggagtttatcgtggtaccagcagaaaccagggaaagcccctaagctcctgatctatcaggcttcccagttgcaaagtggggtcccatcacgtttcagtggcagtggatttgggacagatttcactcttaccatcagcagtctgcaacctgaagattttgctacgtactactgtcaacagaggcattctccgcctcatacgttcggccaagggaccaaggtggaaatcaaacgg

SEQ ID NO: 15 - DNA, 324 bp, Homo sapiens
gacatccagatgacccagtctccatcctccctgtctgcatctgtaggagaccgtgttaccatcacttgccgggcaagtcagagtattaatgttcggttaatttggtaccagcagaaaccagggaaagaccctaagctcctgatctattcttcttcccatttgcaaagtggggtcccatcacgtttcagtggcagtggatctgggacagatttcactctcaccatcagcagtctgcaacctgaagattttgctacgtactactgtcaacagtatcattatacgccttttacgttcggccaagggaccaaggtggaaatcaaacgg

SEQ ID NO: 16 - DNA, 324 bp, Homo sapiens
gacatccagatgacccagtctccatcctccctgtctgcatctgtaggagaccgtgtcaccatcacttgccgggcaagtcagtatattgggattgatttacagtggtatcagcagaaaccagggaaagcccctgagctcctgatctatcggggttcctttttgcacagtggggtcccatcacgtttcagtggcagtggatctgggacagatttcactctcaccatcagcagtctgcaacctgaagattttgctacgtactactgtcaacagcggtggattcggcctcatacgttcggccaagggaccaaggtggaaatcaaacgg

SEQ ID NO: 17 - DNA, 324 bp, Homo sapiens
gacatccagatgacccagtctccatcctccctgtctgcatctgtaggagaccgtgtcaccatcacttgccgggcaagtcaggtgattggtagtcggttaatttggtaccagcagaaaccagggaaagaccctaagctcctgatctatcgtgcttcccggttgcaaagtggggtcccatcacgtttcagtggcagtggatctgggacagatttcactctcaccatcagcagtctgcaacctgaagattttgctacgtactactgtcaacagtataagttggatccttatacgttcggccaagggaccaaggtggaaatcaaacgg

SEQ ID NO: 18 - DNA, 323 bp, Homo sapiens
gacatccagatgacccagtctccatcctccctgtctgcatctgtaggagaccgtgtcaccatcacttgccgggcaagtcagcctattttgtttagtttaaattggtaccagcagaaaccagggaaagcccctaagctcctgatctatagtgcgtcctcgttgcaaagtggggtctcatcacgtttcagtggcagtggatttgggacagatttcactctcaccatcagcagtctgcaacctgaagatttcgctacgtactactgtcaacagcatcattcgcggccttatacgttcggccaagggaccaaggtggaaatcaaacg

SEQ ID NO: 19 - DNA, 324 bp, Homo sapiens
gacatccagatgacccagtctccatcctccctgtctgcatctgtaggggaccgtgtcaccatcacttgccgggcaagtcagaatattgcgtggcagttaaggtggtaccagcagaaaccagggaaagcccctacgctcctgatctatgctacttcccagttgcaaagtggggtcccatcacgtttcagtggcagtggatctgggacagatttcactctcaccatcagcagtctgcaacctgaagattttgctacgtactactgtcaacagacggcgtcttttcctgttacgttcggccaagggaccaaggtggaaatcaaacgg

SEQ ID NO: 20 - DNA, 324 bp, Homo sapiens
gacatccagatgacccagtctccatcctccctgtctgcatctgtaggagaccgtgtcaccatcacttgccgggcaagtcaggagattcatgattatttaagttggtaccagcagaaaccagggaaagcccctaagctcctgatctatctgtcttcccgtttgcaaagtggggtcccatcacgtttcagtggcagtagatctgggacagatttcactctcaccatcagcagtctgcaacctgaagattttgctacgtactactgtcaacagtatcatttggggccttatacgttcggccaagggaccaaggtggaaatcaaacgg

SEQ ID NO: 21 - DNA, 324 bp, Homo sapiens
gacatccagatgacccagtctccaccctccctgtctgcatctgtaggagaccgtgtcaccatcacttgccgggcaagtcagcatatttataatgctttaaggtggtaccagcagaaaccagggaaagcccctaagctcctgatctatgcttcttccaagttgcaaagtggggtcccatcacgtttcagtggcagtggatctgggacagatttcactctcaccatcagcagtctgcaacctgaagatttcgctacgtactactgtcaacagcattattctactccttatacgttcggccaagggaccaaggtggaaatcaaacgg

SEQ ID NO: 22 - DNA, 323 bp, Homo sapiens
gacatccagatgacccagtctccatcctccctatctgcatccgtaggagaccgtgtcaccatcacttgccgggcaagtcagatgattaggaattatttactttggtaccagcaggcaccagggaaagcccctaagctcctgatctataatgcttccaagttgcaaagtggggtcccatcacgtttcagtggcagtggatctgggacagattttactctcaccatcagcagtctgcaacctgaagattttgctacgtactactgtcaacaggcgcatactgctccttttacgttcggccaagggaccaaggtggaaatcaaacg

SEQ ID NO: 23 - DNA, 324 bp, Homo sapiens
gacatccagatgacccagtctccatcctccctgtctgcatctgtaggagaccgtgtcaccatcacttgccgggcaagtcagagtattaggaggtatttaacttggtaccagcagaaaccagggaaagcccctaagctcctgatctattcggcttcccatttgcaaagtggggtcccattacgtttcagtggcagtggatttgggacagatttcactctcaccatcagcagtctgcaacctgaagattttgctacgtactactgtcaacaggcttatattgcgccttttacgttcggccaagggaccaaggtggaaatcaaacgg

SEQ ID NO: 24 - DNA, 322 bp, Homo sapiens
gacatccagatgacccagtctccatcctccctgtctgcatctgtaggagaccgtgtcaccatcacttgccgggcaagtcagcggattggtaggtatttaaattggtaccagcagaaaccagggaaagcccctgagctcctgatctattgggtttcccggttgcaaagtggggtcccatcacgtttcagtggcagtggatttgggacagatttcactctcaccatcagcagtctgcaacctgaagattttgctacgtactactgtcaacaggtgcatagttttcctatgacgttcggccaagggaccaaggtggaaatcaaac

SEQ ID NO: 25 - DNA, 324 bp, Homo sapiens
gacatccagatgacccagtctccatcctccctgtctgcatctgtaggagaccgtgtcaccatcacttgccgggcaagttctccgattaattggagtttaaattggtaccagcagaaaccagggaaagcccctaagctcctgatcgggttggggtccgttttgcaaagtggggtcccatcacgtttcagtggcagtggatctgggacagatttcactctcaccatcagcagtctgcaacctgaagattttgctacgtactactgtgcgcagagtgggagggggcctgagacgttcggccaagggaccaaggtggaaatcaaacgg

SEQ ID NO: 26 - DNA, 324 bp, Homo sapiens
gacatccagatgacccagtctccatcctccctgtctgcatctgtaggagaccgtgtcaccatcacttgccgggcaagtcaggagattcatgattatttaagttggtaccagcagaaaccagggaaagcccctaagctcctgatctatctgtcttcccgtttgcaaagtggggtctcatcacgtttcagtggcagtggatctgggacagatttcactctcaccatcagcagtctgcaacctgaagattttgctacgtactactgtcaacagtattatggttatccttatacgttcggccaagggaccaaggtggaaatcaaacgg

SEQ ID NO: 27 - DNA, 323 bp, Homo sapiens
gacatccagatgacccagtctccatcctccctgtctgcatctgtaggagaccgtgtcaccatcacttgccgggcaagtcagtatattgggcgtcatttagtgtggtaccagcagaaaccagggaaagcccctaagctcctgatctattttgcgtccatgttgcaaagtggggtcccatcacgtttcagtggcagtggatctgggacagatttcactctcaccatcagcagtctgcaacctgaagattttgctacgtaccactgtcaacaggttcattttgatccttttacgttcggccaagggaccaaggtggaaatcaaacg

SEQ ID NO: 28 - DNA, 323 bp, Homo sapiens
gacatccagatgacccagtctccatcctccctgtctgcatctgtaggagaccgtgtcaccatcacttgccgggcaagtcagccgattcatgattatttaacttggtatcagcagaaaccagggaaagcccctaagctcctgatctatttggcgtcccgtttgcaaagtggggtcccatcacgtttcagtggcagtggatctgggacagatttcactctcaccatcagcagtctgcaacctgaagatttcgctacgtactactgtcaacagtatcatgtgctgccttatacgttcggccaagggaccaaggtggaaatcaaacg

SEQ ID NO: 29 - DNA, 324 bp, Homo sapiens; identical to SEQ ID NO: 12

SEQ ID NO: 30 - DNA, 324 bp, Homo sapiens
gacatccagatgacccagtctccatcctccctgtctgcatctgtaggagaccgtgtcaccatcacttgccgagcaagtcagaggatttctacgtatttaaattggtaccagcagaaaccagggaaagcccctaagctcctgatctatcgtagttccatgttgcaaagtggggtcccatcacgtttcagtggcagtggatctgggacagatttcactctcaccatcagcagtctgcaacctgaagatttcgctacgtactactgtcaacagtattctttttctcctcttacgttcggccaagggaccaaggtggaaatcaaacgg

SEQ ID NO: 31 - DNA, 324 bp, Homo sapiens
gacatccagatgacccagtctccatcctccctgtctgcatctgtaggagaccgtgtcaccatcacttgccgggcaagtcagaatattaagaggtatttatattggtaccagcagaaaccagggaaagcccctaagctcctgatctatcatatttccactttgcaaagtggggtcccatcacgtttcagtggcagtggatctgggacagatttcactctcaccatcagcagtctgcaacctgaagattttgctacgtactactgtcaacagtcttttcgggctcctattacgttcggccaagggaccaaggtggaaatcaaacgg

SEQ ID NO: 32 - DNA, 324 bp, Homo sapiens
gacatccagatgacccagtctccatcctccctgtctgcatctgtaggagaccgtgtcaccattacttgccgggcaagtcagcatattgggagtatgttagagtggtaccagcagaaaccagggaaagcccctaagctcctgatctatcgtgcgtcctttttgcaaagtggggtcccatcacgtttcagtggcagtggatctgggacagatttcactctcaccatcagcagtctgcaacctgaagattttgctacgtactactgtcaacaggggcgggcgcttccttttacgtttggccaagggaccaaggtggaaatcaaacgg

SEQ ID NO: 33 - DNA, 324 bp, Homo sapiens
gacatccagatgacccagtctccatcctccctgtctgcatctgtaggagaccgtgtcaccatcacttgccgggcaagtcaggatattggtacggcgttattgtggtaccagcagaaaccagggaaagaccctaggctcctgatctataggggttcccatttgcaaagtggggtcccatcacgtttcagtggcagtggatctgggacagatttcactctcaccatcagcagtctgcaacctgaagattttgctacgtactcctgtcaacagtatcggtatgagcctatgacgttcggccaagggaccaaggtggaaatcaaacgg

SEQ ID NO: 34 - DNA, 324 bp, Homo sapiens
gacatccagatgacccagtctccatcctccctgtctgcatctgtaggagaccgtgtcaccatcacttgccgggcaagtcagcctattcagggttggttaaattggtatcagcagaaaccagggaaagcccctaagctcctgatctattattcttccctgttgcaaagtggggtcccatcacgtttcagaggcagtggatctgggacagatttcactctcaccatcagcagtctgcaacctgaagatttcgctacgtactactgtcaacagagggaggtgaagccttttacgttcggccaagggaccaaggtggaaatcaaacgg

SEQ ID NO: 35 - DNA, 323 bp, Homo sapiens
gacatccagatgacccagtctccatcctccctgtctgcatctgtaggagaccgtgtcaccatcacttgccgggcaagtcagcggattagtcatgcgttacggtggtatcagcagaaaccagggaaagcccctaagctcctgatctatcgtgcttccgctttgcaaagtggggtcccatcacgtttcagtggcagtggatctgggacagatttcactctcaccatcagcagtctgcaacctgaagattttgctacgtactactgtcaacagaatcgttcggtgccttttacgttcggccaagggaccaaggtggaaatcagacg

SEQ ID NO: 36 - DNA, 322 bp, Homo sapiens
gacatccagatgacccagtctccatcctccctgtctgcatctgtaggagaccgtgtcaccatcacttgccgggcaagtcagaatattcgtaggtatttagtttggtatcagcagaaaccagggaaagcccctaagctcctgatctataatgcgtcccatttgcaaagtggggtcccatcacgtttcagtggcagtggatctgggacagatttcactctcaccatcagcagtctgcaacctgaagattttgctacgtactactgtcaacagatttatctttctccttttacgttcggccaagggaccaaggtggaaatcaaac

SEQ ID NO: 37 - DNA, 322 bp, Homo sapiens
gacatccagatgacccagtctccatcctccctgtctgcatctgtaggagaccgtgtcaccatcacttgccgggcaagtcagagtattgggcgttatatatattggtaccagcagaaaccagggaaagcccctaagctcctgatctataatgtttcctatttgcaaagtggggtcccatcacgtttcagtggcagtggatctgggacagatttcactctcaccatcagcagtctgcaacctgaagattttgctacgtactactgtcaacagtgttttcgggggccttgtacgttcggccaagggaccaaggtggaaatcaaac

SEQ ID NO: 38 - DNA, 324 bp, Homo sapiens
gacatccagatgacccagtctccatcctccctgtctgcatctgtaggagaccgtgtcaccatcacttgccgggcaagtcaggatattggtgataggttacggtggtatcagcagaaaccagggaaagcccctaagctcctgatctatcatgggtccaggttggaaagtggggtcccatcacgtttcagtggcagtagatctgggacagatttcactcttaccatcagcagtctgcaacctgaagattttgctacgtactactgtcaacagcagtggtttcgtccttatacgttcggccaagggaccaaggtggaaatcaaacgg

SEQ ID NO: 39 - DNA, 324 bp, Homo sapiens
gacatccagatgacccagtctccatcctccctgtctgcatctgtaggagaccgtgtcaccatcacttgccgggcaagtcagtcgattgggaataatttactttggtaccagcagaaaccagggaaagcccctaagctcctgatctattatacgtccaggttgcaaagtggggtcccatcacgtttcagtggcagtggatctgggacagatttcactctcaccatcagcagtctgcaacctgaagattttgctacgtactactgtcaacagcgtcggactcatcctcatacgttcggccaagggaccaaggtggaaatcaaacgg

SEQ ID NO: 40 - DNA, 324 bp, Homo sapiens
gacatccagatgacccagtctccatcctccctgtctgcatctgtaggagaccgtgtcaccatcacttgccgggcaagtcaggatatttttactaagttaaggtggtaccagcagaaaccagggaaagcccctaggctcctgatctatgcgggttcccgtttgcaaagtggggtcccatcacgtttcagtggcagtggatctgggacagatttcactctcaccatcagcagtctgcaacctgaagattttgctacgtactactgtcaacaggttaagcagaagccttggacgttcggccaagggaccaaggtggaaatcaaacgg

SEQ ID NO: 41 - DNA, 324 bp, Homo sapiens
gacatccagatgacccagtctccatcctccctgtctgcatctgtaggagaccgtgtcaccatcacttgccgggcaagtcagaatattgagtcttggttaaggtggtaccagcagaaaccagggaaagcccctaagctcctgatctatcattcgtccaggttgcaaagtggggtcccatcacgtttcagtggcagtggatctgggacagatttcactctcaccatcagcagtctgcaacctgaagattttgctacgtactactgtcaacagtctagggttcgtccttttacgttcggccaagggaccaaggtggaaatcaaacgg

SEQ ID NO: 42 - DNA, 324 bp, Homo sapiens
gacatccagatgacccagtctccatcctccctgtctgcatctgtaggagaccgtgtcaccatcacttgccgggcaagtcagagcattagcagctatttaaattggtaccagcagaaaccagggaaagcccctaagctcctgatcaggcgtggttcccttttgcaaagtggggtcccatcacgtttcagtggcagtggatctgggacagatttcactctcaccatcagcagtctgcaacctgaagattttgctacgtactactgtcgtcagggtatggctcgtccttggacgttcggccaagggaccaaggtggaaatcaaacgg

SEQ ID NO: 43 - DNA, 324 bp, Homo sapiens
gacatccagatgacccagtctccatcctccctgtctgcatctgtaggagaccgtgtcaccatcacttgccgggcaagtcagaatattgataggaggttactttggtaccagcagaaaccagggaaagcccctaagctcctgatctatggttcttccaagttgcaaagtggggtcccatcacgtttcagtggcagtggatctgggacagatttcactctcaccatcagcagtctgcaacctgaagattttgctacgtactactgtcaacagaggatttatgatcctcatacgttcggccaagggaccaaggtggaaatcaaacgg

SEQ ID NO: 44 - DNA, 324 bp, Homo sapiens
gacatccagatgacccagtctccatcctccctgtctgcatctgtaggagaccgtgtcaccatcacttgccgggcaagtcagtctatttcgaagaatttactttggtatcagcagaaaccagggaaagcccctaagctcctgatctatcattcttcctttttgcaaagtggggtcccatcacgttttagtggcagtggatctgggacagatttcactctcaccatcagcagtctgcaacctgaagattttgctacgtactactgtcaacagcgttttcggtatcctcatacgttcggccaagggaccaaggtggaaatcaaacgg

SEQ ID NO: 45 - DNA, 324 bp, Homo sapiens
gacatccagatgacccagtctccatcctccctgtctgcatctgtaggagaccgtgtcaccatcacttgccgggcaagtcagactattcgtaagaggttacattggtaccagcagaaaccagggaaagcccctaagctcctgatctatcatgcgtccaagttgcaaagtggggtcccatcacgtttcagtggcagtggatctgggacagatttcactctcaccatcagcagtctgcaacctgaagattttgctacgtactactgtcaacagcgttctgatcctccttatacgttcggccaagggaccaaggtggaaatcaaacgg

SEQ ID NO: 46 - DNA, 324 bp, Homo sapiens
gacatccagatgacccagtccccatcctccctgtctgcatctgtaggagaccgtgtcaccatcacttgccgggcaagtcaggagattaggaagcggttaaggtggtaccagcagaaaccagggaaagcccctaagctcctgatctatcgggcttccactttgcaaagtggggtcccatcacgtttcagtggcagtggatctgggacagatttcactctcaccattagcagtctgcaacctgaagattttgctacgtactactgtcaacagctttttcagtcgccttggacgttcggccaagggaccaaggtagaaatcaaacgg

SEQ ID NO: 47 - DNA, 324 bp, Homo sapiens
gacatccagatgacccagtctccatcctccctgtctgcatctgtaggagaccgtgtcaccatcacttgccgggcaagtcaggagattcataagcgtttactttggtaccagcagaaaccagggaaagcccctaagctcctgatctatagtggttccactttgcaaagtggggtcccatcacgtttcagtggcagtggatctgggacagatttcactctcaccatcagcagtctgcaacctgaagattttgctacgtactactgtcaacagcgttatctgcagcctcatacgttcggccaagggaccaaggtggaaatcaaacgg

SEQ ID NO: 48 - DNA, 324 bp, Homo sapiens
gacatccagatgacccagtctccatcctccctgtctgcatctgtaggagaccgtgtcaccatcacttgccgggcaagtcagcatattggtcgtaggttactgtggtaccagcagaaaccagggaaagcccctaagctcctgatctattatagttccaagttgcaaagtggggtcccatcacgtttcagtggcagtggatctgggacagatttcactctcaccatcagcagcctgcaacctgaagattttgctacgtactactgtcaacagcggactattcagcctcatacgttcggccaagggaccaaggtggaaatcaaacgg

SEQ ID NO: 49 - DNA, 324 bp, Homo sapiens
gacatccagatgacccagtctccatcctccctgtctgcatctgtaggagaccgtgtcaccatcacttgccgggcaagtcagtctatttttaagcggttacggtggtaccagcagaaaccagggaaagcccctaagctcctgatctatgcttcttccgtgttgcaaagtggggtcccatcacgtttcagtggcagtggatctgggacagatttcactctcaccatcagcagtctgcaacctgaagattttgctacgtactactgtcaacagaatgttgctattccttttacgttcggccaagggaccaaggtggaaatcaaacgg

SEQ ID NO: 50 - DNA, 324 bp, Homo sapiens
gacatccagatgacccagtctccatcctccctgtctgcatctgtaggagaccgtgtcaccatcacttgccgggcaagtcaaccgattggtcatcggttacgttggtatcagcagaaaccagggaaagcccctaagctcctgatctatcgggcgtccaagttgcaaagtggggtcccatcacgtttcagtggcagtggatctgggacagatttcactctcaccatcagcagtctgcaacctgaagattttgctacgtactactgtcaacagctttataagcagcctttgacgttcggccaagggaccaaggtggaaatcaaacgg

SEQ ID NO: 51 - DNA, 324 bp, Homo sapiens
gacatccagatgacccagtctccatcctccctgtctgcatctgtaggagaccgtgtcaccatcacttgccgggcaagtcagtggattaatgataggttatcttggtatcagcagaaaccagggaaagcccctaagctcctgatctatcgtaagtccggtttgcaaagtggggtcccatcacgtttcagtggcagtggatctgggacagatttcactctcaccatcagcagtctgcaacctgaagattttgctacgtactactgtcaacagtttcggaatattccttttacgttcggccaagggaccaaggtggaaatcaaacgg

SEQ ID NO: 52 - DNA, 324 bp, Homo sapiens
gacatccagatgacccagtctccatcctccctgtctgcatccgtaggagaccgtgtcaccatcacttgccgggcaagtcagcctattagtaggaggttattgtggtatcagcagaaaccagggaaagcccctaagctcctgatctatggtgcttccaggttgcaaagtggggttccatcacgtttcagtggcagtggatctgggacagatttcactctcaccatcagcagtctgcaacctgaagatttagctacgtactactgtcaacagagggagacgaatcctcatacgttcggccaagggaccaaggtggaaatcaaacgg

SEQ ID NO: 53 - DNA, 324 bp, Homo sapiens
gacatccagatggcccagtctccatcctccctgtctgcatctgtaggagaccgtgtcaccatcacttgccgggcaagtcaggttattggtaaggagttagcttggtaccagcagaaaccagggaaagcccctaagctcctgatctatcatgtgtcccggttgcgaagtggggtcccatcacgtttcagtggcagtggatctgggacagatttcactctcaccatcagcagtctacaacctgaagattctgctacgtactactgtcaacagaaggttgcttatccttttacgttcggccaagggaccaaggtggaaatcaaacgg

SEQ ID NO: 54 - DNA, 324 bp, Homo sapiens
gacatccagatgacccagtctccatcctccctgtctgcatctgtaggagaccgtgtcaccatcacttgccgggcaagtcaggatattgttgataggttatcttggtatcagcagaaaccggggaaagcccctaagctcctgatctatcggtcgtcccggttgcgaagtggggtcccatcacgtttcagtggcagtggatctgggacagatttcactctcaccatcagcagtctgcaacctgaagattttgctacgtactactgtcaacagcgtcttcgttttcctattacgttcggccaagggaccaaggtggaaatcaaacgg

SEQ ID NO: 55 - DNA, 324 bp, Homo sapiens
gacatccagatgacccagtctccatcctccctgtctgcatctgtaggagaccgtgtcaccatcacttgccgggcaagtcaggcgatttggcgttctttaaattggtaccagcagaagccagggaaagcccctaagctcctgatctatcggtcgtcccgtttgcaaagtggggtcccatcacgtttcagtggcagtggatctgggacagatttcactctcaccatcagcagtctgcaacctgaagatttcgctacgtactactgtcaacagtattctaatcggccttatacgttcggccaagggaccaaggtggaaatcaaacgg

SEQ ID NO: 56 - DNA, 324 bp, Homo sapiens
gacatccagatgactcagtctccatcctccctgtctgcatctgtaggagaccgtgtcaccatcacttgccgggcaagtcagaagattgggcagcatttacattggtaccagcagaaaccagggaaagcccctaagctcctgatctatcgtacttccattttgcaaagtggggtcccatcacgtttcagtggcagtggatctgggacagatttcactctcaccatcagcagtctgcaacctgaagattttgctacgtactactgtcaacagaatcataggcgtccttatacgttcggccaagggaccaaggtggaaatcaaacgg

SEQ ID NO: 57 - DNA, 324 bp, Homo sapiens
gacatccagatgacccagtctccatcctccctgtctgcatctgtaggagaccgtgtcaccatcacttgccgggcaagtcagaatattgataggaggttactttggtaccagcagaaaccagggaaagcccctaagctcctgatctatggttcttccaagttgcaaagtggggtcccatcacgtttcagtggcagtcgatctgggacagatttcactctcaccatcagcagtctgcaacctgaagattttgctacgtactactgtcaacagaggatttatgatcctcatacgttcggccaagggaccaaggtggaaatcaaacgg

SEQ ID NO: 58 - DNA, 324 bp, Homo sapiens
gacatccagatgacccagtctccatcctccctgtctgcatctgtaggagaccgtgtcaccatcacttgccgggcaagtcaggatattgggaggaatttattgtggtaccagcagaaaccagggaaagcccctaggctcctgatctattatagttcccggttgcaaagtggggtcccatcacgttttagtggcagtggatctgggacagatttcactctcaccatcagcagtctgcaacctgaagatttagctacgtactactgtcaacagcgttcgcgtaatccttttacgttcggccaagggaccaaggtggaaatcaaacgg

SEQ ID NO: 59 - DNA, 324 bp, Homo sapiens
gacatccaaatgacccagtctccatcctccctgtctgcatctgtaggagaccgtgtcaccatcacttgccgggcaagtcaggatattggtgggaggttacattggtaccagcagaaaccagggaaagcccctaagctcctgatctatcaggcttccaagttgcaaagtggggtcccatcacgtttcagtggcagtggatctgggacagatttcactctcaccatcagcagtctgcaacctgaagactttgctacgtactactgtcaacagaagcggcggcagcctcatacgttcggccaagggaccaaggtggaaatcaaacgg

SEQ ID NO: 60 - DNA, 324 bp, Homo sapiens
gacatccagatgacccagtctccatcctccctgtctgcatctgtaggagaccgtgtcaccatcacttgccgggcaagtcagtcgattgataggcgtttagggtggtaccagcagaaaccagggaaagcccctaagctcctgatctcgggttcttccaggttgcaaagtggggtcccatcacgtttcagtggcagtggatctgggacagatttcactctcaccatcagcagtctgcaacctgaagattttgctacgtactactgtgtgcagcggcagcgtctgccttatacgttcggccaagggaccaaggtggaaatcaaacgg

SEQ ID NO: 61 - DNA, 324 bp, Homo sapiens
gacatccagatgacccagtctccatcctccctgtctgcatctgtaggagaccgtgtcaccatcacttgccgggcaagtcagttgattaataggcgtttatcgtggtaccagcagaaaccagggaaaccccctaagctcctgatctatcatcattccaggttgcaaagtggggtcccatcacgtttcagtggcagtggatctgggacagatttcactctcaccatcagcagtctgcaacctgaagattttgctacgtactactgtcaacagacgcgtattaggcctcatacgttcggccaagggaccaaggtggaaatcaaacgg

SEQ ID NO: 62 - DNA, 324 bp, Homo sapiens; identical to SEQ ID NO: 59

SEQ ID NO: 63 - DNA, 324 bp, Homo sapiens; identical to SEQ ID NO: 59

SEQ ID NO: 64 - DNA, 324 bp, Homo sapiens
gacatccagatgacccagtctccatcctccctgtctgcatctgtaggagaccgtgtcaccatcacttgccgggcaagtcaggagattgataggaggttactgtggtatcagcagaaaccagggaaagcccctaagctcctgatctattctgcttccaggttgcaaagtggggtcccatcacgtttcagtggcagtggatctgggacagatttcactctcaccatcagcagtctgcaacctgaagattttgctacgtactactgtcaacagcggtatcatatgcctcatacgttcggccaagggaccaaggtgaaaatcaaacgg

SEQ ID NO: 65 - DNA, 324 bp, Homo sapiens
gacatccagatgacccagtctccatcctccctgtctgcatctgtaggagaccgtgtcaccatcacttgccgggcaagtcagaagattgggaagcggttacgttggtaccagcagaaaccagggaaagcccctaagctcctgatctatggggcttccaggttgcaaagtggggtcccatcacgtttcagtggcagtggatctgggacagatttcactctcaccatcagcagtctgcaacctgaagattttgctacatactactgtcaacagaatttggagcggcctaatacgttcggccaagggaccaaggtggaaatcaaacgg

SEQ ID NO: 66 - DNA, 324 bp, Homo sapiens
gacatccagatgacccagtctccatcctccctgtctgcatctgtaggagaccgtgtcaccatcacttgccgggcaagtcagccgattgggagtaggatactgtggtaccagcagaaaccagggagagcccctaagctcctgatctatcatgcttccaagttgcaaagtggggtcccatcacgtttcagtggcagtggatctgggacagatttcactctcaccatcagcagtctgcaacctgaagattttgctacgtactactgtcaacagcgtaagtatcagcctcatacgttcggccaaggaaccaaggtggaaatcaaacgg

SEQ ID NO: 67 - DNA, 324 bp, Homo sapiens; identical to SEQ ID NO: 64

SEQ ID NO: 68 - DNA, 324 bp, Homo sapiens
gacatccagatgacccagtctccatcctccctgtctgcatctgtaggagaccgtgtcaccatcacttgccgggcaagtcagatgattgggaagcggttaaggtggtaccagcagaaaccagggaaagcccctaagctcctgatctattttgcttcccggttgcaaagtggggtcccatcacgtttcagtggcagtggatctgggacagatttcactctcaccatcagcagtctgcaacctgaagattttgctacgtactactgtcaacagtctaggcagcatcctcatacgttcggccaagggaccaaggtggaaatcaaacgg

SEQ ID NO: 69 - DNA, 324 bp, Homo sapiens
gacgtccagatgacccagtctccatcctccctgtctgcatctgtaggagaccgtgtcaccatcacttgccgggcaagtcagcttattcgtaagaggttacgttggtaccagcagaaaccagggaaagcccctaagctcctgatctatcattcgtccaagttgcaaagtggggtcccatcacgtttcagtggcagtggatctgggacagatttcactctcaccatcagcagtctgcaacctgaagattttgctacgtactactgtcaacaggggcatagtcggccttttacgttcggccaagggaccaaggtggaaatcaaacgg

SEQ ID NO: 70 - DNA, 324 bp, Homo sapiens
gacatccagatgacccagtccccatcctccctgtctgcatctgtaggagaccgtgtcaccatcacttgccgggcaagtcagcggattcataataggttatcttggtaccagcagaaaccagggaaagcccctaagctcctgatctatgcggcgtccaaattgcaaagtggggtcccatcacgtttcagtggcagtggatctgggacagatttcactctcaccatcagcagtctgcaacctgaagattttgctacgtactactgtcaacagactagttataggcctcatacgttcggccaagggaccaaggtggaaatcaaacgg

SEQ ID NO: 71 - DNA, 324 bp, Homo sapiens
gacatccagatgacccagtctccatcctccctgtctgcatctgtaggagaccgtgtcaccatcacttgccgggcaagtcagaatattaatgagcgtttattgtggtaccagcagaaaccagggaaagcccctacgctcctgatctatcattcgtcccggttgcaaagtggggtcccatcacgtttcagtggcagtggatctgggacagatttcactctcaccatcagcagtctgcaacctgaagattttgctacgtactactgtcaacagaagtataagcgtccttatacgttcggccaagggaccaaggtggaaatcaaacgg

SEQ ID NO: 72 - DNA, 324 bp, Homo sapiens
gacatccagatgacccagtctccatcctccctgtctgcatctgtaggagaccgtgtcaccatcacttgccgggcaagtcagaatattgggcggaagttaaggtggtatcagcagaaaccagggaaagcccctaagctcctgatctatgggacgtcccgtttgcaaagtggggtcccatcacgtttcagtggcagtggatctgggacagatttcactctcaccatcagcagtctgcaacctgaagattttgctacgtactactgtcaacagaatttgcatctgccttctacgttcggccaagggaccaaggtggaaatcaaacgg

SEQ ID NO: 73 - DNA, 324 bp, Homo sapiens
gacatccagatgacccagtctccatcctccctgtctgcatctgtaggagaccgtgtcaccatcacttgccgggcaagtcaggatattgagcggcgtttactgtggtatcagcagaaaccagggaaagcccctaagctcctgatctattcgacgtcccgtttgcaaagtggggtcccatcacgtttcagtggcagtggatctgggacagatttcactctcaccataagcagtctgcaacctgaagattttgctacgtactactgtcaacagaggcatacgtcgcctcatacgttcggccaagggaccaaggtggaaatcaaacgg

SEQ ID NO: 74 - DNA, 324 bp, Homo sapiens
gacatccagatgacccagtctccatcctccctgtctgcatctgtaggagaccgtgtcaccatcacctgccgggcaagtcagaatattactaatcggttacggtggtaccagcagaaaccagggaaagcccctaagctcctgatctatcgtagttccgttttgcaaagtggggtcccatcacgtttcagtggcggtggatctgggacagatttcactctcaccatcagcagtctgcaacctgaagattttgctacgtactactgtcaacagcataattatcagcctcatacgttcggccaagggaccaaggtggaaatcaaacgg

SEQ ID NO: 75 - DNA, 324 bp, Homo sapiens
gacatccagatgacccagtctccatcctccctgtctgcatctgtaggagaccgtgtcaccatcacttgccgggcaagtcagtcgattgggaggggtttagcgtggtatcagcagaaaccagggaaagcccctaagctcctgatctatatggggtcccgtttgcaaagtggggtcccatcacgttttagtggcagtggatctgggacagatttcactctcaccatcagcagtctgcaacctgaagatttcgctacgtactactgtcaacagcagaggcatcttcctcggacgttcggccaagggaccaaggtggaaatcaaacgg

SEQ ID NO: 76 - DNA, 324 bp, Homo sapiens
  misc_feature 322: n = A,T,C or G
gacatccagatgacccagtctccatcctccctgtctgcatctgtaggagaccgtgtcaccatcacttgccgggcaagtcggccgatttctactagtttagtttggtaccagcagaaaccagggaaagcccctaagctcctgatctataatgcgtccaatttgcaaagtggggtcccatcacgtttcagtggcagtggatctgggacagatttcactctcaccatcagcagtctgcaacctgaagattttgctacgtactactgtcaacagtcgcagactcttcctgttacgttcggccaagggaccaaggtggaaatcaaangg

SEQ ID NO: 77 - DNA, 324 bp, Homo sapiens
gacatccagatgacccagtctccatcctccctgtctgcatctgtaggagaccgtgtcaccatcacttgccgggcaagtcagagtattgggcggcggttaaattggtatcagcagaaaccagggaaagcccctaagctcctgatctatcggacgtccacgttgcaaagtggggtcccgtcacgtttcagtggcagtggatctgggacagatttcactctcaccatcagcagtctgcaacctgaagattttgctacgtactactgtcaacagacttctcgtgtgccttatacgttcggccaagggaccaaggtggaaatcaaacgg

SEQ ID NO: 78 - DNA, 324 bp, Homo sapiens
gacatccagatgacccagtctccatcctccctgtctgcatctgtaggagaccgtgttaccatcacttgccgggcaagtcaggatattaagaagcatttattgtggtaccagcagagaccagggaaagcccctaagctcctgatctattatagttcccgtttgcaaagtggggtcccatcacgtttcagtggcagtggatctgggacagatttcactctcaccatcagcagtctgcaacctgaagattttgctacgtactactgtcaacagcggcatcatgatccttttacgttcggccaagggaccaaggtggaaatcaaacgg

SEQ ID NO: 79 - DNA, 324 bp, Homo sapiens
gacatccagatgacccagtctccatcctccctgtctgcatctgtaggagaccgtgtcaccatcacttgccgggcaagtcagtctattgatcggaggttactttggtatcagcagaaaccagggaaagcccctaagctcctgatctatagggcttccaggttgcaaagtggggtcccatcacgtttcagtggcagtggatctgggacagatttcactctcaccatcagcagtctgcaacctgaagattttgctacgtactactgtcaacagacttatgcgcggcctaacacgttcggccaagggaccaaggtggaaatcaaacgg

SEQ ID NO: 80 - DNA, 324 bp, Homo sapiens
gacatccagatgacccagtctccatcctccctgtctgcatctgtaggagaccgtgtcaccatcacttgccgggcaagtcagagtattggtccgtggttaagttggtatcagcagaaaccagggaaagcccctaagctcctgatctatcaggtttcccgtctgcaaagtggggtcccatcacgtttcagtggcagtggatctgggacagatttcactctcaccatcagcagtctgcaacctgaagattttgctacgtactactgtcaacagaatcttgcgcctccttatacgttcggccaagggaccaaggtggaaatcaaacgg

SEQ ID NO: 81 - DNA, 324 bp, Homo sapiens
gacatccagatgacccagtctccatcctccctgtctgcatctgtaggagaccgtgtcaccatcacttgccgggcaagtcagggtattatgtatcatttaaggtggtaccagcagaaaccagggaaagcccctaggctcctgatctatcatgggtccactttgcaaagtggggtcccagcacgtttcagtggcagtggatctgggacagattttactctcaccatcagcagtctgcaacctgaagattttgctacgtactactgtcaacagacttggaatgcgcctttgacgttcggccaagggaccaaggtggaaatcaaacgg

SEQ ID NO: 82 - DNA, 324 bp, Homo sapiens
gacatccagatgacccagtctccatcctccctgtctgcatctgtaggagaccgggtcaccatcacttgccgggcaagtcagggtattggtaatagtttacggtggtatcagcagaaaccagggaaagcccctaagctcctgatctattattcttcccatttgcaaagtggggtcccatcacgtttcagtggcagtggatctgggacagatttcactctcaccatcagcagtctgcaacctgaagattttgctacgtactactgtcaacagattaggacgaagccttttacgttcggccaagggaccaaggtggaaatcaaacgg

SEQ ID NO: 83 - DNA, 324 bp, Homo sapiens
gacatccagatgacccagtctccatcctccctgtctgcatctgtaggagaccgtgtcaccatcacttgccgggcaagtcagaagattatgacgcatttacgttggtatcagcagaaaccagggaaagcccctaagctcctgatctatggtgggtcccatttgcaaagtggggtcccatcacgtttcagtggcagtggatctgggacagatttcactctcaccattagcagtctgcaacctgaagattttgctacgtactactgtcaacagacgtgggtgtcgcctatgacgttcggccaagggaccaaggtggaaatcagacgg

SEQ ID NO: 84 - DNA, 324 bp, Homo sapiens
gacatccagatgacccagtctccatcctccctgtctgcatctgtaggagaccgtgtcaccatcacttgccgggcaagtcagtctattgggacgctgttaaattggtatcagcagaaaccagggaaagcccctaagctcctgatctatgcttcttcccgtttgcaaagtggggtcccatcacgtttcagtggcagtggatctgggacagatttcactctcaccattagcagtctgcaacctgaagattttgctacgtactactgtcaacagatgaatagggttcctattacgttcggccaagggaccaaggtggaaatcaaacgg

SEQ ID NO: 85 - DNA, 324 bp, Homo sapiens
gacatccagatgacccagtctccatcctccctgtctgcatctgtaggagaccgtgtcaccatcacttgccgggcaagtcagtctattgggatgctgttatcgtggtaccagcagaaaccagggaaagcccctaagctcctgatctatgctgtgtcccgtttgcaaagtggggtcccatcacgtttcagtggcagtggatctgggacagatttcactctcaccatcagcagtctgcaacctgaagattatgctacgtactactgtcaacagatgcagcgtcctcctattacgttcggccaagggaccaaggtagaaatcaaacgg

SEQ ID NO: 86 - DNA, 324 bp, Homo sapiens
gacatccagatgacccagtctccatcctccctgtctgcatctgtaggagaccgtgtcaccatcacttgccgggcaagtcagccgattaagatgatgttatcgtggtatcagcagaaaccagggaaagcccctaagctcctgatctataataattccactttgcaaagtggggtcccatcacgtttcagtggcagtggatctgggacagatttcactctcaccatcagcagtctgcaacctgaagattttgctacgtactactgtcaacagtataggaggtggccttatacgttcagccaagggactaaggtggaaatcaaacgg

SEQ ID NO: 87 - DNA, 324 bp, Homo sapiens
gacatccagatgacccagtctccatcctccctgtctgcatctgtaggagaccgtgtcaccatcacttgccgggcaagtcaagatattggtaatatgttagcgtggtatcagcagaaaccagggaaagcccctaagcccctgatctattatgcgtcctatttgcaaagtggggtcccatcacgtttcagtggcagtggatctgggacagatttcactctcaccatcagcagtctgcaacctgaagatttcgctacgtactactgtcaacagatgcgtgattatcctgtgacgttcggccaagggaccaaggtggaaatcaaacgg

SEQ ID NO: 88 - DNA, 324 bp, Homo sapiens
gacatccagatgtcccagtctccatcctccctgtctgcatctgtaggagaccgtgtcaccatcacttgccgggcaagtcaagatattggtaatatgttagcgtggtatcagcagaaaccagggaaagcccctaagcccctgatctattatgcgtcctatttgcaaagtggggtcccatcacgtttcagtggcagtggatctgggacagatttcactctcaccatcagcagtctgcaacctgaagattttgctacgtactactgtcaacagctgggtgcgaagcctcatacgttcggccaagggaccaaggtggaaatcaaacgg

SEQ ID NO: 89 - DNA, 324 bp, Homo sapiens
gacatccagatgacccagtctccatcctccctgtctgcatctgtaggagaccgtgtcaccatcacttgccgggcaagtcagaatattgggggtcgtttagtgtggtaccagcagaaaccagggaaagcccctaagctcctgatctatactccgtcccctttgcaaagtggggtcccatcacgtttcagtggcagtggatctgggacagatttcactctcaccatcagcagtctgcaacctgaagattttgctacgtactactgtcaacagcgtcatacgtcgccttttacgttcggccaagggaccaaggtggaaatcaaacgg

SEQ ID NO: 90 - DNA, 324 bp, Homo sapiens
gacatccagatgacccagtctccatcctccctgtctgcatctgtaggagaccgtgtcaccatcacttgccgggcaagtcagaatattgggggtcgtttagtgtggtaccagcagaaaccagggaaagcccctaagctcctgatctatactccgtcccctttgcaaagtggggtcccatcacgtttcagtggcagtggatctgggacagatttcactctcaccatcagcagtctgctacctgaagattttgctacgtactactgtcaacagcgtcatagtgcgccttatacgttcggccaagggaccaaggtggaaatcaaacgg

SEQ ID NO: 91 - DNA, 324 bp, Homo sapiens
gacatccagatgacccagtctccatcctccctgtctgcatctgtaggagaccgtgtcaccatcacttgccgggcaagtcagaatattgggggtcgtttggtgtggtaccagcagaaaccagggaaagcccctaagctcctgatctatactccgtcccctttgcaaagtggggtcccatcacgtttcagtggcagtggatctgggacagatttcactctcaccatcagcagtctgcaacctgaagattttgctacgtactactgtcaacagaggcagcagcagccttatacgttcggccaagggaccaaggtggaaatcaaacgg

SEQ ID NO: 92 - DNA, 324 bp, Homo sapiens
gacatccagatgacccagtctccatcctccctgtctgcatctgtaggagaccgcgtcaccatcacttgccgggcaagtcagaatattgggggtcgtttagtgtggtaccagcagaaaccagggaaagcccctaagctcctgatctatactccgtcccctttgcaaagtggggtcccatcacgtttcagtggcagtggatctgggacagatttcactctcaccatcagcagtctgcaacctgaagattttgctacgtactactgtcaacagagggcttctcggccttatacgttcggccaagggaccaaggtggaaatcaaacgg

SEQ ID NO: 93 - DNA, 324 bp, Homo sapiens
gacatccagatgacccagtctccatcctccctgtctgcatctgtaggagaccgtgtcaccatcacttgccgggcaagtcagaatattgggggtcgtttagtgtggtaccagcagaaaccagggaaagcccctaagctcctgatctatactccgtcccctttgcaaagtggggtcccatcacgtttcagtggcagtggatctgggacagatttcactctcaccatcagcagtctgcaacctgaagattttgctacgtactactgtcaacagcgttatgtgcagccttatacgttcggccaagggaccaaggtggaaatcaaacgg

SEQ ID NO: 94 - DNA, 324 bp, Homo sapiens
gacatccagatgacccagtctccatcctccctgtctgcatctgtaggagaccgtgtcaccatcacttgccgggcaagtcagaatattgggggtcgtttagtgtggtaccagcagaaaccagggaaagcccctaagctcctgatctatactccgtcccctttgcaaagtggggtcccatcacgtttcagtggcagtggatctgggacagatttcactctcaccatcagcagtctgcaacctgaagattttgctacgtactactgtcaacagcgttataagccgccttatacgttcggccaagggaccaaggtgaaaatcaaacgg

SEQ ID NO: 95 - DNA, 324 bp, Homo sapiens
gacatccagatgacccagtctccatcctccctgtctgcatctgtaggagaccgtgtcaccatcacttgccgggcaagtcagaatattgggggtcgtttagtgtggtaccagcagaaaccagggaaagcccctaagctcctgatctatactccgtcccctttgcaaagtggggtcccatcacgtttcagtggcagtggatctgggacagatttcactctcaccatcagcagtctgcaacctgaagattttgctacgtactactgtcaacagcgggttagggcgccttatacgttcggccaagggaccaaggtggaaatcaaacgg

SEQ ID NO: 96 - DNA, 324 bp, Homo sapiens
gacatccagatgacccagtctccatcctccctgtctgcatctgtaggagaccgtgtcaccatcacttgccgggcaagtcagaatattggttctaagttagtgtggtatcagcagaaaccagggaaagcctctaagctcctgatctatactccttccaggttgcaaagtggggtcccatcacgtttcagtggcagtggatctgggacagatttcactctcaccatcagcagtctgcaacctgaagatttcgctacgtactactgtcaacagcggtttatgactccttatacgttcggccaagggaccaaggtggaaatcaaacgg

SEQ ID NO: 97 - DNA, 324 bp, Homo sapiens
gacatccagatgacccagactccatcctccctgtctgcatctgtaggagaccgtgtcaccatcacttgccgggcaagtcagaatattgggaagcagttattgtggtaccagcagaaaccagggaaagcccctaggctcctgatctattgtcctcccccgttgcaaagtggggtcccatcacgtttcagttgcagtggatctgggacagatttcactctcaccatcagcagtctgcaacctgaagattttgctacgtactactgtcaacagcatgcttctaggccttttacgttcggccaagggaccaaggtggaaatcaaacgg

SEQ ID NO: 98 - DNA, 324 bp, Homo sapiens
gacatccagatgacccagtctccatcctccctgtctgcatctgtaggagaccgtgtcaccatcacttgccgggcaagtcagaatattgggggtcgtttagtgtggtaccagcagaaaccagggaaagcccctaagctcctgatctatactccgtcccctttgcaaagtggggtcccatcacgtttcagtggcagtggatctgggacagatttcactctcaccatcagcagtctgcaacctgaagattctgctacgtactactgtcaacagcgttattcgctgccttttacgttcggccaagggaccaaggtggaaatcaaacgg

SEQ ID NO: 99 - DNA, 324 bp, Homo sapiens
gacatccagatgacccagtctccatcctccctgtctgcatctgtaggagaccgtgtcaccatcagttgccgggcaagtcagaatattggtacgcagttacattggtatcagcagaaaccagggaaagcccctaggctcctgatctatggtagttcctttttgcaaagtggggtcccatcacgtttcagtggcagtggatctgggacagatttcactctcaccatcagcagtctgcaacctgaagattttgctacgtactactgtcaacaggttatgttggggcctacgacgttcggccaagggaccaaggtggaaatcaaacgg

SEQ ID NO: 100 - DNA, 324 bp, Homo sapiens
gacatccagatgacccagtctccatcctccctgtctgcatctgtaggagaccgtgtcaccatcacttgccgggcaagtcagaatattcatgggatgttaaggtggtaccagcaaaaaccagggaaagcccctaagctcctgatctatacgccgtccccttcccaaagtggggtcccatcacgtttcagtggcagtggatctggcacagatttcactctcaccatcagcagtctgcaacctgaagattttgctacgtactactgtcaacagactgctacttggccttttacgttcggccaagggaccaaggtggaaatcaaacgg

SEQ ID NO: 101 - DNA, 324 bp, Homo sapiens
gacatccagatgacccagtcaccatcctccctgtctgcatctgtaggagaccgtgtcaccatcacttgccgagcaagtcagcctattgggaataagttacgttggtaccagcagaaaccagggaaagcccctaagctcctgatctatagtccgtccccgttgcaaagtggggtcccatcacgtttcagtggcagtggatctgggacagattacactctcaccatcagcagtctgcaacctgaagattttgctacgtactactgtcaacagacttggtcttttcctggtacgttcggccaagggaccaaggtggaaatcaaacgg

SEQ ID NO: 102 - DNA, 324 bp, Homo sapiens
  misc_feature 275: n = A,T,C or G
gacatccagatgacccagtctccatcctccctgtctgcatctgtaggagaccgtgtcaccatcacttgccaggcaagtcagcctattgatgggaggttagtttggtaccagcagaaaccagggaaagcctctaagctcctgatctatgttccgtccgggttgcaaagtggggtcccatcacgtttcagtggcagtggatctgggacagatttcactctcaccatcagcagtctgcaacctgaagattttgctacgtactactgtcaacagcggcntactcctccttatacgttcggccaagggaccaaggtggaaatcaaacgg

SEQ ID NO: 103 - DNA, 324 bp, Homo sapiens
gacatccagatgacccagtctccatcctccctgtctgcatctgtaggagaccgtgtcaccatcacttgccgggcaagtcagtctattgggggtcgtttagtgtggtaccagcagaaaccagggaaagcccctaagctcctgatctatactccgccccctttgcaaagtggggtcccatcacgtttcagtggcagtggatctgggacagatttcactctcaccatcagcagtctgcaacctgaagattttgctacgtactactgtcaacagcggtatcttaggccttttacgttcggccaagggaccaaggtggaaatcaaacgg

SEQ ID NO: 104 - DNA, 324 bp, Homo sapiens
gacatccagatgacccagtccccatcctccctgtctgcatctgtaggagaccgtgtcaccatcacttgccgggcaagtcggaatattgggggtcgtttagtgtggtaccagcagaaaccagggaaagcccctaagctcctgatctatactccgtcccctttgcaaagtggggtcccatcacgtttcagtggcagtggatctgggacagatttcactctcaccatcagcagtctgcaacctgaagattttgctacgtactactgtcaacagcggcataatgagccttttacgttcggccaagggaccaaggtggaaatcaaacgg

SEQ ID NO: 105 - DNA, 324 bp, Homo sapiens
  misc_feature 90, 102, 108, 151, 153, 183, 273, 287, 288: n = A,T,C or G
gacatccagatgacccagtctccatcctccctgtctgcatctgtaggagaccgtgtcaccatcacttgccgggcaagtcagaatattttnactttgttaaantggtancagcagaaaccagggaaagcccctaagctcctgatctatgctncntcccgtttgcaaagtggggtcccatcacgnttcagtggcagtggatctgggacagatttcactctcaccatcagcagtctgcaacctgaagattttgctacgtactactgtcaacaggcntataggcatcctannacgctcggccaagggaccaaggtggaaatcaaacgg

SEQ ID NO: 106 - DNA, 324 bp, Homo sapiens
gacatccagatgacccagtctccatcctccctgtctgcatctgtgggagaccgtgtcaccatcacttgccgggcaagtcaggatattaagtcgcatttacgttggtatcagcagaaaccagggaaagcccctaagctcctgatctatactccttcctctttgcaaagtggggtcccatcacgtttcagtggcagtggatctgggacagatttcactctcaccatcagcagtctgcaacctgaagattctgctacgtactactgtcaacaggtgttgacggttccttatacgttcggccaagggaccaaggtggaaatcaaacgg

SEQ ID NO: 107 - DNA, 324 bp, Homo sapiens
gacatccagatgacccagtctccatcctccctgtctgcatctgtaggagaccgtgtcaccatcacttgccgggcaagtcaggatattgggcgttggttatcgtggtaccagcagaaaccagggaaagcccctaagctcctgatctatgcgggttcccagttgcaaagtggggtcccatcacgtttcagtggcagtggatctgggacagatttcactctcaccatcagcagtctgcaacctgaagattttgctacgtactactgtcaacggtcgtgggatcctcctacgacgttcggccaagggaccaaggtggaaatcaaacgg

SEQ ID NO: 108 - DNA, 324 bp, Homo sapiens
gacatccagatgacccagtctccatcctccctgtctgcatctgtaggagaccgtgtcaccatcacttgccgggcaagtcagccgattgggagtatgttagtgtggtatcagcagaaaccagggaaagcccctaagctcctgatctatacgccgtcctctttgcaaagtggggtcccatcacgtttcagtggcagtggatctgggacagatttcactctcaccatcagcagtctgcaacctgaagattttgctacgtactactgtcaacagaagtatatggagcctcatacgttcggccaagggaccaaggtggaaatcaaacag

SEQ ID NO: 109 - DNA, 324 bp, Homo sapiens
gacatccagatgacccagtccccatcctccctgtctgcatctgtaggagaccgtgtcaccatcacttgccgggcaagtcaggatattaatcgtcagttagtttggtatcagcagaaaccagggaaagcccctaagctcctgatctatactccgtcctcgttgcaaagtggggtcccatcacgtttcagtggcagtggatctgggacagatttcactctcaccatcagcagtctgcaacctgaagattatgctacgtactactgtcaacagaagtatcgttatccttttacgttcggccaagggaccaaggtggaaatcaaacgg

SEQ ID NO: 110 - DNA, 324 bp, Homo sapiens
gacatccagatgacccagtctccatcctccctgtctgcatctgtaggagaccgtgtcaccatcacttgccgggcaagtcaggatattagtcggtttttaaattggtatcagcagaaaccagggaaagcccctaagctcctgatctattggacgtccttgttgcaaagtggggtcccatcacgcttcagtggcagtggatctgggacagatttcactctcaccatcagcagtctgcaacctgaagattttgctacgtactactgtcaacagagtaggcatcatcctactacgttcggccaagggaccaaggtggaaatcaaacgg

SEQ ID NO: 111 - DNA, 324 bp, Homo sapiens; identical to SEQ ID NO: 88

SEQ ID NO: 112 - DNA, 324 bp, Homo sapiens
gacatccagatgacccagtctccatcctccctgtctgcatctgtaggagaccgtgtcaccatcacttgccgggcaagtcaggagattaataatatgttagtgtggtaccagcagaaaccagggaaagcccctaagctcctgatctatgcgccttccggtttgcaaagtggggtcccatcacgtttcagtggcagtggatctgggacagatttcactctcaccatcagcagtctgcaacctgaagattttgctacgtactactgtcaacagaggaggtatcctccttttacgttcggccaagggaccaaggtggaaatcaaacgg

SEQ ID NO: 113 - DNA, 324 bp, Homo sapiens
gacatccagatgacccagtctccatcctccctgtctgcatctgtaggagaccgtgtcaccatcacttgccgggcaagtcaggagattggtagtcatttacgttggtatcagcagaaaccagggaaagcccctaagctcctgatctatcaggagtcccagttgcaaagtggggtcccatcacgtttcagtggcagtggatctgggacagatttcactctcaccatcagcagtctgcaacctgaagattttgctacgtactactgtcaacagacttggaattcgcctatgacgttcggccaagggaccaaggtggaaatcaaacgg

SEQ ID NO: 114 - DNA, 324 bp, Homo sapiens
gacatccagatgacccagtctccatcctccctgtctgcatctgtaggagaccgtgtcaccatcacttgccgggcaagtcaggggattgggcggcatttacgttggtaccagcagaaaccagggaaagcccctaagcttctgatctattcgccttccgggttgcaaggtggggtcccatcacgtttcagtggcagtggatctgggacagattacactctcaccatcagcagtctgcaacctgaagattatgctacgtactactgtcaacaggtatattcgcctccttttacgttcggccaagggaccaaggtggaaatcaaacgg

SEQ ID NO: 115 - DNA, 324 bp, Homo sapiens
gacatccagatgacccagtctccatcctccctgtctgcatctgtaggagaccgtgtcaccatcacttgccgggcaagtcagaagattgggaatatgttagcttggtatcagcagaaaccagggaaagcccctaagctcctgatctataagtattccaagttgcaaagtggggtcccatcacgtttcagtggcagtggatctgggacagatttcactctcactatcagcagtctgcaacctgaagattttgctacgtactactgtcaacagcttgctgtgcctcctcatacgttcggccaaggaactaaggtggaaatcaaacgg

SEQ ID NO: 116 - DNA, 324 bp, Homo sapiens
gacatccagatgacccagtctccatcctccctgtctgcatctgtaggagaccgtgtcaccatcacttgccgggcaagtcagtatattcagatgcggttacggtggtaccagcagaaaccagggaaagcccctaggctcctgatctatggtgcttccatgttgcaaagtggggtcccatcacgtttcagtggcagtggatctgggacagatttcactctcaccatcagcagtctgcaacctgaagattttgctacgtactactgtcaacaggattggactgcgcctcatacgttcggccaagggaccaaggtggaaatcaaacgg

SEQ ID NO: 117 - DNA, 324 bp, Homo sapiens
gacatccagatgacccagtctccatcctccctgtctgcatctgtaggagaccgtgtcaccatcacttgccgggcaagtcagcagattggtcagctgttaaattggtaccagcagaaaccagggaaagcccctaagctcctgatctatgcgggttcccggttgcaaagtggggtcccatcacgtttcagtggcagtggatctgggacagatttcactctcaccatcagcagtttgcaacctgaagattttgctacgtactactgtcaacagatgcggcagacgcctgtgacgttcggccaagggaccaaggtggaaatcaaacgg

SEQ ID NO: 118 - DNA, 324 bp, Homo sapiens
gacatccagatgacccagtctccatcctccctgtctgcatctgtaggagaccgtgtcaccatcacttgccgggcaagtcaggatattgggcagctgttaaattggtatcagcagaaaccagggaaagcccctaagctcctgatctatgcttcgtcccgtttgcaaagtggggtcccatcacgtttcagtggcagtggatctgggacagatttcactctcaccatcagcagtctgcaacctgaagattttgctacgtactactgtcaacagcggacgtataatccttctacgttcggcccagggaccaaggtggaaatcaaacgg

SEQ ID NO: 119 - DNA, 324 bp, Homo sapiens
gacatccagatgacccagtctccatcctccctgtctgcatctgtaggagaccgtgtcaccatcacttgccgggcaagtcaggatattggtgctttgttacggtggtaccagcagaaaccagggaaagcccctaagctcctgatctatactccgtccgagttgcaaagtggggtcccatcacgtttcagtggcagtggatctgggacagatttcactctcaccatcagcagtctgcaacctgaagattttgctacgtactactgtcaacaggtttttcgttctccttatacgttcggccaagggaccaaggtggaaatcaaacgg

SEQ ID NO: 120 - DNA, 324 bp, Homo sapiens
gacatccagatgacccagtctccatcctccctgtctgcatctgtaggagaccgtgtcaccatcacttgccgggcaagtcaggatattggggctcagttaaggtggtaccggcagaaaccagggaaagcccctaagctcctgatctatgcgccttccgctttgcaaagtggggtcccgtcacgtttcagtggcagtggatctgggacagatttcactctcaccatcagcagtctgcaacctgaagattttgctacgtactactgtcaacaggtggcgcttcgtccttatacgttcggccaagggaccagggtggagatcaaacgg

SEQ ID NO: 121 - DNA, 324 bp, Homo sapiens
gacatccagatgacccagtctccatcctccctgtctgcgtctgtaggagaccgtgttaccatcacttgccgggcaagtcaggatattgggcataagttacgttggtatcagcagaaaccagggaaagcccctaagctcctgatctatacgccttccactttgcaaagtggggtcccatcacgtttcagtggcagtggatctgggacagatttcactctcaccatcagcagtctgcaacctgaagatttcgctacgtactactgtcaacagacttggactcctccttatacgttcggccaagggaccaaggtggaaatcaaacgg

SEQ ID NO: 122 - DNA, 324 bp, Homo sapiens
gacatccagatgacccagtctccatcctccctgtctgcatctgtaggagaccgtgtcaccatcacttgccgggcaagtcaggatattgatacgcatttacgttggtaccagcagaaaccagggaaagcccctaagctcctgatctatgggagttcctttttgcaaagtggggtcccatcacgtttcagtggcagtggatctgggacagatttcactctcaccatcagcagtctgcaacctgaagatttcgctacgtactactgtcaacagacgtgggcgcgtcctatgacgttcggccaagggaccaaggtggaaatcaaacgg

SEQ ID NO: 123 - DNA, 324 bp, Homo sapiens
gacatccagatgacccagtctccatcctccctgtctgcatctgtaggagaccgtgtcaccatcacttgccgggcaagtcaggatattaaggggatgttagtttggtatcagcagaaaccagggaaagcccctaagctcctgatctatacgccgtccaggttgcaaagtggggtcccatcacgtttcagtggcagtggatctgggacagatttcactctcaccatcagcagtctgcaacctgaagattttgctacgtactactgtcaacagacttgggtgtctcctcagacgttcggccaagggaccaaggtggaaatcaaacgg

SEQ ID NO: 124 - DNA, 324 bp, Homo sapiens
gacatccagatgacccagtctccatcctccctgtctgcatctgtaggagaccgtgtcaccatcacttgccgggcaagtcaggatattaagtcgcatttacgttggtatcagcagaaaccagggaaagcccctaagctcctgatctatactccttcctctttgcaaagtggggtcccatcacgtttcagtggcagtggatctgggacagatttcactctcaccatcagcagtctgcaacctgaagattttgctacgtactactgtcaacaggtgtcttcgacgccttatacgttcggccaagggaccaaggtggaaatcaaacgg

SEQ ID NO: 125 - DNA, 324 bp, Homo sapiens; identical to SEQ ID NO: 113

SEQ ID NO: 126 - DNA, 324 bp, Homo sapiens; identical to SEQ ID NO: 88

SEQ ID NO: 127 - DNA, 324 bp, Homo sapiens
gacatccagatgacccagtctccatcctccctgtctgcatctgtaggagaccgtgtcaccatcacttgccgggcaagtcaggagattgggggtaatttagtgtggtatcagcagaaaccagggaaagcccctaagctcctgatctatgctccttccaggttgcaaagtggggtcccatcacgtttcagtagcagtggatctgggacagatttcactctcaccatcagcagtctgcaacctgaagattttgctacgtactactgtcaacagaagtttagttatccttttacgttcggccaagggaccaaggtggaaatcaaacgg

SEQ ID NO: 128 - DNA, 324 bp, Homo sapiens; identical to SEQ ID NO: 112

SEQ ID NO: 129 - DNA, 324 bp, Homo sapiens
gacatccagatgacccagtctccatcctccctgtctgcatctgtaggagaccgtgtcaccatcacttgccgggcaagtcagtggattggtaatcatttacgttggtaccagcagaaaccagggaaagcccctacgctcctgatctatggcagttccaggttgcaaagtggggtcccatcacgtttcagtggcagtggatctgggacagatttcactctcaccatcagcagtctgcaacctgaagattttgctacgtactactgtcaacagacttggaattctcctatgacgttcggccaagggaccaaggtggaaatcaaacgg

SEQ ID NO: 130 - DNA, 324 bp, Homo sapiens
gacatccagatgacccagtctccatcctccctgtctgcatctgtaggagaccgtgtcaccatcacttgccgggcaagtcaggctattgatattcatttacgttggtaccagcagaaaccagggaaagcccctaagctcctgatctatcatgcgtcctccttgcaaagtggggtcccatcacgtttcagtggcagtggatctgggacagatttcactctcaccatcagcagtctgcaacctgaagattttgctacgtactactgtcaacagacgtatcgttctcctatgacgttcggccaagggaccaaggtggaaatcaaacgg

SEQ ID NO: 131 - DNA, 324 bp, Homo sapiens
  misc_feature 150: n = A,T,C or G
gacatccagatgacccagtctccatcctccctgtctgcatctgtaggagaccgtgtcaccatcacttgccgggcaagtcaggcgattggtcagtctttaaggtggtaccagcagaaaccagggaaagcccctacgctcctgatctatcanagttccaatttgcaaagtggggtcccatcacgtttcagtggcagtggatctgggacagatttcactctcaccatcagcagtctgcaacctgaagattttgctacgtactactgtcaacagacttgggtttctcctatgacgttcggccaagggaccaaggtggaaatcaaacgg

SEQ ID NO: 132 - DNA, 324 bp, Homo sapiens
gacatccagatgacccagtctccatcctccctgtctgcatctgtaggagaccgtgtcaccatcacttgccgggcaagtcagtatattggtggtagtttaaggtggtaccagcagaaaccagggaaagcccctaagctcctgatctatagtggttccactttgcaaagtggggtcccatcacgtttcagtggcagtggatctgggacagatttcactctcaccatcagcagtctgcaacctgaagatttcgctacgtactactgtcaacagacttgggtgtctcctatgacgttcggccaagggaccaaggtggaaatcaaacgg

SEQ ID NO: 133 - DNA, 324 bp, Homo sapiens
  misc_feature 114: n = A,T,C or G
gacatccagatgacccagtctccatcctccctgtctgcatctgtaggagaccgtgtcaccatcacttgccgggcaagtcagtatattaatgctcatttacgttggtaccagcanaaaccagggaaagcccctaagctcctgatctatatgtcttcctatttgcaaagtggggtcccatcacgtttcagtggcagtggatctgggacagatttcactctcaccattagcagtctgcaacctgaagattttgctacgtactactgtcaacagacttggtcttctcctatgacgttcggccaagggaccaaggtggaaatcaaacgg

SEQ ID NO: 134 - DNA, 324 bp, Homo sapiens
gacatccagatgacccagtctccatcctccctgtctgcatctgtaggagaccgtgtcaccatcacttgccgggcaagtcaggtgattggtaatgcgttacgttggtaccagcagaaaccagggaaagcccctaagctcctgatctattatgggtcctatttgcaaagtggggtcccatcacgtttcagtggcagtggatctgggacagatttcactctcaccatcagcagtctgcaacctgaagattttgctacgtactactgtcaacagattcattttaagccttttacgttcggccaagggaccaaggtggaaatcaaacgg

SEQ ID NO: 135 - DNA, 324 bp, Homo sapiens
gacatccagatgacccagtctccatcctccctgtctgcatctgtaggagaccgtgtcaccattacttgccgggcaagtcagcggattggtcatcatttaaggtggtatcagcagaaaccagggaaagcccctaagctcctgatctattcggcttccgctttgcaaagtggggtcccatcacgtttcagtggcagtggatctgggacagatttcactctcaccatcagcagtctgcaacctgaagattttgctacgtactactgtcaacagacgtggaatgctcctatgacgttcggccaagggaccaaggtggaaatcaaacgg

SEQ ID NO: 136 - DNA, 324 bp, Homo sapiens
gacatccagatgacccagtctccatcctccctgtctgcatctgtaggagaccgtgtcaccatcacttgccgggcaagtcagaggattgggttgatgttaaattggtatcagcagaaaccagggaaagcccctaggctcctgatctatgcggcttccaggttgcaaagtggggtcccatcacgtttcagtggcagtggatctgggacagatttcactctcaccatcagcagtctgcaacctgaagattttgctacgtactactgtcaacagatgttgcatcctcctgtgacgttcggccaagggaccaaggtggaaatcaaacgg

SEQ ID NO: 137 - DNA, 324 bp, Homo sapiens
  misc_feature 9, 114: n = A,T,C or G
gacatccanatgacccagtctccatcctccctgtctgcatctgtaggagaccgtgtcaccatcacttgccgggcaagtcagaggattgaggggaagttattgtggtaccagcanaaaccagggaaagcccctaagctcctgatctattgtccgtccaatttgcaaagtggggtcccatcacgtttcagttgcagtggatctgggacagatttcactctcaccatcagcagtctgcaacctgaagattttgctacgtactactgtcaacagaagtttcgtgagccttctacgttcggccaagggaccaaggtggaaatcaaacgg

SEQ ID NO: 138 - DNA, 324 bp, Homo sapiens
gacatccagatgacacagtctccatcctccctgtctgcatctgtaggagaccgtgtcaccatcacttgccgggcaagtcagtctattgggggtcgtttagtgtggtaccagcagaaaccagggaaagcccctaagctcctgatctatactccgccccctttgcaaagtggggtcccatcacgtttcagtggcagtggatctgggacagatttcactctcaccatcagcagtctgcaacctgaagattttgctacgtactactgtcaacagcggtatcttaggccttttacgttcggccaagggaccaaggtggaaatcaaacgg

SEQ ID NO: 139 - DNA, 324 bp, Homo sapiens
gacatccagatgacccagtctccatcctccctgtctgcatctgtaggagaccgtgtcaccatcacttgccgggcaagtcagagtattggtgtgaatttattgtggtaccagcagataccagggaaagcccctaggctcctgatctatggtgcttcctatttgcaaagtggggtcccatcacgtttcagtggcagtggatctgggacagatttcactctcaccatcagcagtctgcaacctgaagattttgctacgtactactgtcaacagtatttttttgctcctttgacgttcggccaagggaccaaggtggaaatcaaacgg

SEQ ID NO: 140 - DNA, 324 bp, Homo sapiens
gacatccagatgacccagtctccatcctccctgtctgcatctgtaggagaccgtgtcaccatcacttgccgggcaagtcagtcgattggtcataatttagtttggtatcagcagaaaccagggaaagcccctaagctcctgatctatactccgtcccctttgcaaagtggggtcccatcacgtttcagtggcagtggatctgggacagatttcactctcaccatcagcagtctgcaacctgaagattttgctacgtactactgtcaacagaagtatactccgccttatacgtttggccaagggaccaaggtggaaatcaaacgg

SEQ ID NO: 141 - DNA, 324 bp, Homo sapiens
  misc_feature 49: n = A,T,C or G
gacatccagatgacccagtctccatcctccctgtctgcatctgtagganaccgtgtcaccatcacttgccgggcaagtcagtctattggggtgcagttaaggtggtaccagcagaaaccagggaaagcccctaagctcctgatctatcatgggtcccagttgcaaagtggggtcccatcacgtttcagtggcagtggatctgggacagatttcactctcaccatcagcagtctgcaacctgaagattttgctacgtactactgtcaacagaattgggctcgtcctattacgttcggccaagggaccaaggtggaaatcaaacgg

SEQ ID NO: 142 - DNA, 324 bp, Homo sapiens
gacatccagatgacccagtctccatcctccctgtctgcatctgtaggggaccgtgtcaccatcacttgccgggcaagtcagtctattgcgacgtctttacggtggtatcagcagaaaccagggaaagcccctaagctcctgatctatcattcgtccgtgttgcaaagtggggtcccatcacgtttcagtggcagtggatctgggacagatttcactctcaccatcagcagtctgcaacctgaagattttgctacgtactactgtcaacagacttgggttgtgcctatgacgttcggccaagggaccaaggtggaaatcaaacgg

SEQ ID NO: 143 - DNA, 324 bp, Homo sapiens; identical to SEQ ID NO: 132

SEQ ID NO: 144 - DNA, 324 bp, Homo sapiens
gacatccagatgacccagtctccatcctccctgtctgcatctgtaggagaccgtgtcaccatcacttgccgggcaagtcagtcgattaaggggcatttagtttggtaccagcagaaaccagggaaagcccctatgctcctgatctatagtccgtcctctttgcgaagtggggtcccatcacgtttcagtggcagtggatctgggacagatttcactctcaccatcagcagtctgcaacctgaagattttgctacgtactactgtcaacaggtttatgagaagccttttacgttcggccaagggaccaaggtggaaatcaaacgg

SEQ ID NO: 145 - DNA, 324 bp, Homo sapiens
  misc_feature 279: n = A,T,C or G
gacatccagatgacccagtctccatcctccctgtctgcatctgtaggagaccgtgtcaccatcacttgccgggcaagtcagccgattcatggtgcgttacggtggtaccagcagaaaccagggaaagcccctatgctcctgatctatactccttcccagttgcaaagtggggtcccatcacgtttcagtggcagtggatctgggacagatttcactctcaccatcagcagtctgcaacctgaagattttgctacgtactactgtcaacaggtgggtcanaagccttatacgttcggccaagggaccaaggtggaaatcaaacgg

SEQ ID NO: 146 - DNA, 324 bp, Homo sapiens
gacatccagatgacccagtctccatcctccctgtctgcatctgtaggagaccgtgtcaccatcacttgccaggcaagtcagcctattgatgggaggttagtttggtaccagcagaaaccagggaaagcctctaagctcctgatctatgttccgtccgggttgcaaagtggggtcccatcacgtttcagtggcagtggatctgggacagatttcactctcaccatcagcagtctgcaacctgaagattttgctacgtactactgtcaacagcggcatactcctccttatacgttcggccaagggaccaaggtggaaatcaaacgg

SEQ ID NO: 147 - DNA, 324 bp, Homo sapiens
gacatccagatgacccagtctccatcctccctgtctgcatctgtaggagaccgtgtcaccatcacttgccgggcaagtcagcctattaataattggttaaattggtaccagcagaaaccagggaaagcccctaagctcctgatctatgctacgtcccggttgcaaagtggggtcccatcacgtttcagtggcagtggatctgggacagatttcactctcaccatcagcagtctgcaacctgaagattttgctacgtactactgtcaaccgagttggactcctcctcctacgttcggccaagggaccaaggtggaaatcaaacgg

SEQ ID NO: 148 - DNA, 324 bp, Homo sapiens; identical to SEQ ID NO: 108

SEQ ID NO: 149 - DNA, 324 bp, Homo sapiens; identical to SEQ ID NO: 117

SEQ ID NO: 150 - DNA, 324 bp, Homo sapiens
  misc_feature 244: n = A,T,C or G
gacatccagatgacccagtctccatcctccctgtctgcatctgtaggagaccgtgccaccatcacttgccgggcaagtcagcagattggtgctcatttacggtggtatcagcagaaaccagggaaagcccctaagctcctgatctatcagtcgtcccagttgcaaagtggggtcccatcacgtttcagtggcagtggatctgggacagatttcactctcaccatcagcagtctgcaacctgaanattttgctacgtactactgtcaacagacttgggcgagtcctatgacgttcggccaagggaccaaggtggaaatcaaacgg

SEQ ID NO: 151 - DNA, 324 bp, Homo sapiens; identical to SEQ ID NO: 95

SEQ ID NO: 152 - DNA, 324 bp, Homo sapiens
  misc_feature 81: n = A,T,C or G
gacatccagatgacccagtctccatcctccctgtctgcatctgtaggagaccgtgtcaccatcacttgccgggcaagtcanaatattgggggtcgtttagtgtggtaccagcagaaaccagggaaagcccctaagctcctgatctatactccgtcccctttgcaaagtggggtcccatcacgtttcagtggcagtggatctgggacagatttcactctcaccatcagcagtctgcaacctgaagattttgctacgtactactgtcaacagaggagtgtttctccttatacgttcggccaagggaccaaggtggaaatcaaacgg

SEQ ID NO: 153 - DNA, 324 bp, Homo sapiens
  misc_feature 82, 84, 89, 96, 101, 108, 277, 278: n = A,T,C or G
gacatccagatgacccagtctccatcttccctgtctgcatctgtaggagaccgtgtcaccatcacttgccgggcaagtcagntnattgntacttcnttaangtggtancagcagaaaccagggaaagcccctacgctcctgatctataattcttcccagttgcaaagtggggtcccatcacgtttcagtggcagtggatctgggacagatttcactctcaccatcagcagtctgcaacctgaagattttgctacgtactactgtcaacagacgtggnntcgtcctatgacgttcggccaagggaccaaggtggaaatcaaacgg

SEQ ID NO: 154 - DNA, 324 bp, Homo sapiens
gacatccagatgacccagtctccatcctccctgtctgcatctgtaggagaccgtgtcaccatcacttgccgggcaagtcagaatattgggggtcgtttagtgtggtaccagcagaaaccagggaaagcccctaagctcctgatctatactccgtcccctttgcaaagtggggtcccatcacgtttcagtggcagtggatctgggacagatttcactctcaccatcagcagtctgcaacctgaagattttgctacgtactactgtcaacagcgtcattatccgccttttacgttcggccaagggaccaaggtggaaatcaaacgg

SEQ ID NO: 155 - DNA, 324 bp, Homo sapiens
  misc_feature 9: n = A,T,C or G
gacatccanatgacccagtctccatcctccctgtctgcatctgtaggagaccgtgtcaccatcacttgccgggcaagtcagaatattgggggtcgtttagtgtggtaccagcagaaaccagggaaagcccctaagctcctgatctatactccgtcccctttgcaaagtggggtcccatcacgtttcagtggcagtggatctgggacagatttcactctcaccatcagcagtctgcaacctgaagattttgctacgtactactgtcaacagcgtcatacgagtccttatacgttcggccaagggaccaaggtggaaatcaaacgg

SEQ ID NO: 156 - DNA, 324 bp, Homo sapiens
  misc_feature 114: n = A,T,C or G
gacatccagatgacccagtctccatcctccctgtctgcatctgtaggagaccgtgtcaccatcacttgccgggcaagtcagaatattgggggtcgtttagtgtggtaccagcanaaaccagggaaagcccctaagctcctgatctatactccgtcccctttgcaaagtggggtcccatcacgtttcagtggcagtggatctgggacagatttcactctcaccatcagcagtctgcaacctgaagattttgctacgtactactgtcaacagaggcattctgagccttggacgttcggccaagggaccaaggtggaaatcaaacgg

SEQ ID NO: 157 - DNA, 324 bp, Homo sapiens
gacatccagatgacccagtctccatcctccctgtctgcatctgtaggagaccgtgtcaccatcacttgccgggcaagtcagaatattgggggtcgtttagtgtggtaccagcagaaaccagggaaagcccctaagctcctgatctatactccgtcccctttgcaaagtggggtcccatcacgtttcagtggcagtggatctgggacagatttcactctcaccatcagcagtctgcaacctgaagattttgctacgtactactgtcaacagcgttctaagcttccttatacgttcggccaagggaccaaggtggaaatcaaacgg

SEQ ID NO: 158 - DNA, 324 bp, Homo sapiens
gacatccagatgacccagtctccatcctccctgtctgcatctgtaggagaccgtgtcaccatcacttgccgggcaagtcagaatattgggggtcgtttagtgtggtaccagcagaaaccagggaaagcccctaagctcctgatctatactccgtcccctttgcaaagtggggtcccatcacgtttcagtggcagtggatctgggacagatttcactctcaccatcagcagtctgcaacctgaagattttgctacgtactactgtcaacagaagtttaagcagccttatacgttcggccaagggaccaaggtggaaatcaaacgg

SEQ ID NO: 159 - DNA, 324 bp, Homo sapiens
gacatccagatgacccagtctccatcctccctgtctgcatctgtaggagaccgtgtcaccatcacttgccgggcaagtcagaatattgggggtcgtttagtgtggtaccagcagaaaccagggaaagcccctaagctcctgatctatactccgtcccctttgcaaagtggggtcccatcacgtttcagtggcagtggatctgggacagatttcactctcaccatcagcagtctgcaacctgaagattttgctacgtactactgtcaacagcggtttagtagtccttttacgtttggccaagggaccaaggtggaaatcaaacgg

SEQ ID NO: 160 - DNA, 324 bp, Homo sapiens
  misc_feature 9, 168, 244: n = A,T,C or G
gacatccanatgacccagtctccatcctccctgtctgcatctgtaggagaccgtgtcaccatcacttgccgggcaagtcagaatattgggggtcgtttagtgtggtaccagcagaaaccagggaaagcccctaagctcctgatctatactccgtcccctttgcaaagnggggtcccgtcacgtttcagtggcagtggatctgggacagatttcactctcaccatcagcagtctgcaacctgaanattttgctacgtactactgtcaacagagggctgttactccttttacgttcggccaagggaccaaggtggaaatcaaacgg

SEQ ID NO: 161 - DNA, 324 bp, Homo sapiens
  misc_feature 81: n = A,T,C or G
gacatccagatgacccagtctccatcctccctgtctgcatctgtaggagaccgtgtcaccatcacttgccgggcaagtcanaatattgggggtcgtttagtgtggtaccagcagaaaccagggaaagcccctaagctcctgatctatactccgtcccctttgcaaagtggggtcccatcacgtttcagtggcagtggatctgggacagatttcactctcaccatcagcagtctgcaacctgaagattttgctacgtactactgtcaacagcgtgctacgcagccttatacgttcggccaagggaccaaggtggaaatcaaacgg

SEQ ID NO: 162 - DNA, 324 bp, Homo sapiens
gacatccagatgacccagtctccatcctccctgtctgcatctgtaggagaccgtgtcaccatcacttgccgggcaagtcagaatattgggggtcgtttagtgtggtaccagcagaaaccagggaaagcccctaagctcctgatctatactccgtcccctttgcaaagtggggtcccatcacgtttcagtggcagtggatctgggacagatttcactctcaccatcagcagtctgcaacctgaagattttgctacgtactactgtcaacagcggaaggctcctccttatacgttcggccaagggaccaaggtggaaatcaaacgg

SEQ ID NO: 163 - DNA, 324 bp, Homo sapiens
gacatccagatgacccagtctccatcctccctgtctgcatctgtaggagaccgtgtcaccatcacttgccaggcaagtcagaatattggggttcttttaaattggtaccagcagaaaccagggaaagcccctaagctcctgatctatgctagttccaggttgcaaagtggggtcccatcacgtttcagtggcagtggatctgggacagatttcactctcaccatcagcagtctgcaacctgaagattttgctacgtactactgtcaacagcgtaattttcctcctcctacgttcggccaagggaccaaggtggaaatcaaacgg

SEQ ID NO: 164 - DNA, 324 bp, Homo sapiens; identical to SEQ ID NO: 97

SEQ ID NO: 165 - DNA, 324 bp, Homo sapiens
  misc_feature 9: n = A,T,C or G
gacatccanatgacccagtctccatcctccctgtctgcatctgtaggagaccgtgtcaccatcacttgccgggcaagtcagaatattcatgggatgttaaggtggtaccagcaaaaaccagggaaagcccctaagctcctgatctatacgccgtccccttcccaaagtggggtcccatcacgtttcagtggcagtggatctggcacagatttcactctcaccatcagcagtctgcaacctgaagattttgctacgtactactgtcaacagactgctacttggccttttacgttcggccaagggaccaaggtggaaatcaaacgg

SEQ ID NO: 166 - DNA, 324 bp, Homo sapiens; identical to SEQ ID NO: 107

SEQ ID NO: 167 - DNA, 324 bp, Homo sapiens
gacatccagatgacccagtctccatcctccctgtctgcatctgtaggagaccgtgtcaccatcacttgccgggcaagtcaagatattggtaatatgttagcgtggtatcagcagaaaccagggaaagcccctaagcccctgatctattatgcgtcctatttgcaaagtggggtcccatcacgtttcagtggcagtggatctgggacagatttcactctcaccatcagcagtctgcaacctgaagattttgctacgtactactgtcaacagatgcggaatttgcctcggacgttcggccaagggaccaaggtggaaatcaaacgg

SEQ ID NO: 168 - DNA, 324 bp, Homo sapiens
gacatccagatgacccagtctccatcctccctgtctgcatctgtaggagaccgtgtcaccatcacttgccgggcaagtcaggatatttttccttttttaaattggtaccagcagaaaccagggaaagcccctgagctcctgatctatagggcttccattttgcacagtggggtcccatcacgtttcagtggcagtggatctgggacagatttcactctcaccatcagcagtctgcaacctgaagattttgctacgtactactgtcaacagattgcgaggtctcctcgtacgttcggccaagggaccaaggtggaaatcaaacgg

SEQ ID NO: 169 - DNA, 324 bp, Homo sapiens
gacatccagatgacccagtctccatcctccctgtctgcatctgtaggagaccgtgtcaccatcacttgccgggcaagtcggttgattggtaagcatttaagttggtatcagcagaaaccagggaaagcccctaagctcctgatctatcgttcgtccgttttgcaaagtggggtcccatcacgtttcagtggcagtggatctgggacagatttcactctcaccatcagcagtctgcaacctgaagattttgctacgtactactgtcaacagcatgctacttcgcctaggacgttcggccaagggaccaaggtggaaatcaaacgg

SEQ ID NO: 170 - DNA, 324 bp, Homo sapiens
gacatccagatgacccagtctccatcctccctgtctgcatctgtaggagaccgtgtcaccatcacttgccgggcaagtcagaagattggtagtcatttatcgtggtatcagcagaaaccagggaaagcccctaagctcctgatctatcgtacttcccagttgcaaagtggggccccatcacgtttcagtggcagtggatctgggacagatttcactctcaccatcagcagtctgcaacctgaagattttgctacgtactactgtcaacagcaggcgaagtcgcctaggacgttcggccaagggaccaaggtggaaatcaaacgg

SEQ ID NO: 171 - DNA, 324 bp, Homo sapiens
gacatccagatgacccagtctccatcctccctgtctgcatctgtaggagaccgtgttaccatcacttgccgggcaagtcagcagattgatgattatttaaattggtaccagcagaaaccagggaaagcccctaagctcctgatctattggacttccttgttgcaaagtggggtcccatcacgtttcagtggcagtggatctgggacagatttcactctcaccatcagcagtctgcaacctgaagattttgctacgtactactgtcaacagagtgctcataggccttttacgttcggccaagggaccaaggtggaaatcaaacgg

SEQ ID NO: 172 - DNA, 324 bp, Homo sapiens
gacatccagatgacccagtctccatcctccctgtctgcatctgtaggagaccgcgtcaccatcacttgtcgggcaagtcagaatatttcgtatcatttagtgtggtaccagcagaaaccagggaaagcccctaagctcctgatctattcttcttccaatttgcaaagtggggtcccatcacgtttcagtggcagtggatctgggacagatttcactctcaccatcagcagtctgcaacctgaagatttagctacgtactactgtcaacagctggcgagttggcctcatacgctcggccaagggaccaaggtagaaatcaaacgg

SEQ ID NO: 173 - DNA, 324 bp, Homo sapiens
gacatccagatgacccagtctccatcctccctgtctgcatctgtaggagaccgtgtcaccatcacttgccgggcaagtcagaatattagtcgtgggttaaggtggtatcagcagaaaccagggaaagcccctaagctcctgatctatcatgcgtccaagttgcaaagtggggtcccatcacgtttcagtggcagtggatctgggacagatttcaccctcaccatcagcagtctgcaacctgaagattttgctacgtactactgtcaacagtataaggtgtttcctggtacgttcggccaagggaccaaggtggaaatcaaacgg

SEQ ID NO: 174 - DNA, 324 bp, Homo sapiens
gacatccagatgacccagtctccatcctccctgtctgcatctgtaggagaccgtgtcaccatcacttgccgggcaagtcaggagatttcgggggagttaacttggtatcagcagaaaccagggaaagcccctaagctcctgatctactttagttccattttgcaaagtggggtcccatcacgtttcagtggcagtggatctgggacagatttcactctcaccatcagcagtctgcaacctgaagattttgctacgtactactgtcaacagaggaagcttcgtccttatacgttcggccaagggaccaaggtggaaatcaaacgg

SEQ ID NO: 175 - DNA, 324 bp, Homo sapiens
gacatccagatgacccagtctccatcctccctgtctgcatctgtaggagaccgtgtcaccatcacttgccgggcaagtcaggagattgggcagtggttaaattggtaccagcagaaaccagggaaagcccccaagctcctgatctattggggttccgagttgcaaagtggggtcccatcacgtttcagtggcagtggatctgggacagatttcactctcaccatcagcagtctgcaacctgaagattttgctacgtactactgtcaacaggttagtaggaatccttttacgttcggccaagggaccaaggtggaaatcaaacgg

SEQ ID NO: 176 - DNA, 324 bp, Homo sapiens
gacatccagatgacccagtctccatcctccctgtctgcatctgtaggagaccgtgtcaccatcacttgccgggcaagtcagccgattggttcgttgttagagtggtatcagcagaaaccagggaaagcccctaagctcctgatctataatgtttcccgtttgcaaagtggggtcccatcacgtttcagtggcagtggatctgggacagatttcactctcaccatcagcagtctgcaacctgaagatttcgctacgtactactgtcaacagcgtcggtttgctcctcgtacgttcggccaagggaccaaggtggaaatcaaacgg

SEQ ID NO: 177 - DNA, 324 bp, Homo sapiens
gacatccagatgacccagtctccatcctccctgtctgcatctgtaggagaccgtgtcaccatcacttgccgggcaagtcagaatattgatttggagttatcgtggtatcagcagaaaccagggaaagcccctaagctcctgatctattttacttccgttttgcaaagtggggtcccttcacgtttcagtggcagtggatctgggacagatttcactctcaccatcagcagtctgcaacctgaagattatgctacgtactactgtcaacagaggattcggcggccttatacgttcggccaagggaccaaggtggaaatcaaacgg

SEQ ID NO: 178 - DNA, 324 bp, Homo sapiens
gacatccagatgacccagtctccatcctccctgtctgcatctgtaggagaccgtgtcaccatcacttgccgggcaagtcagagcattagcagctatttaaattggtaccagcagaaaccagggaaagcccctaagctcctgatctatgctgcatccagtttgcaaagtggggtcccatcacgtttcagtggcagtggatctgggacagatttcactctcaccatcagcagtctgcaacctgaagattttgctacgtactactgtcaacagagttacagtacccctaatacgttcggccaagggaccaaggtggaaatcaaacgg

SEQ ID NO: 179 - DNA, 324 bp, Homo sapiens
gacatccagatgacccagtctccatcctccctgtctgcatctgtaggagaccgtgtcaccatcacttgccgggcaagtcagaatattattgattatttaaattggtaccagcagaaaccagggaaagcccctaagctcctgatctattggggttcccttttgcaaagtggggtcccatcacgtttcagtggcagtggatctgggacagatttcactctcaccatcagcagtctgcaacctgaagattttgctacgtactactgtcaacagacttataggcgtccttttacgttcggccaagggaccaaggtggaaatcaaacgg

SEQ ID NO: 180 - DNA, 324 bp, Homo sapiens
gacatccagatgacccagtctccatcctccctgtctgcatctgtaggagaccgtgtcaccatcacttgccgggcaagtcagcctattgatgagtggttagtgtggtatcagcagaaaccagggaaagcccctaagctcctgatctatcggggttcccttttgcaaagtggggtcccatcacgtttcagtggcagtggatctgggacagatttcactctcaccatcagcagtctgcaacctgaagattttgctacgtactactgtcaacagtatcggcagatgcctgctacgttcggccaagggaccaaggtggaaatcaaacgg

SEQ ID NO: 181 - DNA, 324 bp, Homo sapiens
gacatccagatgacccagtctccatcctccctgtctgcatctgtaggagaccgtgtcaccatcacttgccgggcaagtcagcctattgcgagtcggttactgtggtaccagcagaaaccagggaaagcccctaagctcctgatctattatgggtccgttttgcaaagtggggtcccatcacgtttcagtggcagtggatctgggacagatttcactctcaccatcagcagtctacaacctgaagattttgctacgtactactgtcaacagacgtgggctcatcctattacgttcggccaagggaccaaggtggaaatcaaacgg

SEQ ID NO: 182 - DNA, 324 bp, Homo sapiens
gacatccagatgacccagtctccatcctccctgtctgcatctgtaggagaccgtgtcaccatcacttgccgggcaagtcagcctatttataagatgttacggtggtaccagcagaaaccaggggaagcccctaagctcctgatctatcaggcttccaatttgcaaagtggggtcccatcacgtttcagtggcagtggatctgggacagatttcactctcaccatcagcggtctgcaacctgaagatttagctacgtactactgtcaacagtttgctaagtggccttatacgttcggccaagggaccaaggtggaaatcaaacgg

SEQ ID NO: 183 - DNA, 324 bp, Homo sapiens
gacatccagatgacccagtctccatcctccctgtctgcatctgtaggagaccgtgtcaccatcacttgccgggcaagtcagccgattaatacgagtttaaattggtatcagcagaaaccagggaaagcccctaagctcctgatctatggggggtcctggttgcaaagtggggtcccatcacgtttcagtggcagtggatctgggacagatttcactctcaccatcagcagtctgcaacctgaagattttgctacgtactactgtcaacagtatctttattctccttctacgttcggccaagggaccaaggtggaaatcaaacgg

SEQ ID NO: 184 - DNA, 324 bp, Homo sapiens
gacatccagatgacccagtctccatcctccctgtctgcatctgtgggagaccgtgtcaccatcacttgccgggcaagtcagccgattcatgagaatttagattggtaccagcagaaaccagggaaagcccctaagctcctgatctatggggcttccatgttgcaaagtggggtcccatcacgtttcagtggcagtggatctgggacagatttcactctcaccatcagcagtctgcaacctgaagattctgctacgtactactgtcaacaggggtgggtttatcctcagacgttcggccaagggaccaaggtggaaatcaaacgg

SEQ ID NO: 185 - DNA, 324 bp, Homo sapiens
gacatccagatgacccagtctccatcctccctgtctgcatctgtaggagaccgtgtcaccatcacttgccgggcaagtcagccgattgatacttttttacggtggtatcagcagaaaccagggaaagcccctaagctcctgatctatcgggcgtcccagttgcaaagtggggtcccatcacgtttcagtggcagtggatctgggacagatttcactctcaccatcagcagtctgcaacctgaagattttgctacgtactactgtcaacagtgggctaggtcgccttttacgttcggccaagggaccaaggtgaaaatcaaacgg

SEQ ID NO: 186 - DNA, 324 bp, Homo sapiens
gacatccagatgacccagtctccatcctccctgtctgcatctgtaggagaccgtgtcaccatcacttgccgggcaagtcagtttattgagtggtatttagcttggtaccagcagaaaccagggaaagcccctaagctcctgatctataatgggtccgttttgcaaagtggggtcccatcacgtttcagtggcagtggatctgggacagatttcactctcaccatcagcagtctgcaacctgaagattttgctacgtactactgtcaacagcgggttgctcgtccttttacgttcggccaagggaccaaggtggaaatcaaacgg

SEQ ID NO: 187 - DNA, 324 bp, Homo sapiens
gacatccagatgacccagtctccatcctccctgtctgcatctgtaggagaccgtgtcaccatcacttgccgggcaagtcagtatattggtactgcgttagattggtaccagcagaaaccagggaaagcccctaagctcctgatctatgcggtttccttgttgcaaagtggggtcccatcacgtttcagtggcagtggatctgggacagatttcactctcactatcagcagtctgcaacctgaggatttagctacgtactactgtcaacaggcgtttgcgccgcctatgacgttcggccaagggaccaaggtggaaatcaaacgg

SEQ ID NO: 188 - DNA, 324 bp, Homo sapiens
gacatccagatgacccagtctccatcctccctgtctgcatctgtaggagaccgtgtcaccatcacttgccgggcaagtcagcatattacggatcagttacggtggtaccagaagaaaccagggaaagcccctaagctcctgatctatagtgcttccattttgcaaagtggggtcccatcacgtttcagtggcagtggatctgggacagatttcactctcaccatcagcagtctgcaacctgaagattttgctacgtactactgtcaacagatttatattcggcctggtacgttcggccaagggaccaaggtggaaatcaaacgg

SEQ ID NO: 189 - DNA, 324 bp, Homo sapiens
gacatccagatgacccagtctccatcctccctgtctgcatctgtaggagaccgtgtcaccatcacttgccgggcaagtcagcatattggtgattatttagcgtggtatcagcagaaaccagggaaagcccctaagctcctgatctatccgagttcccagttgcaaagtggggtcccatcacgtttcagtggcagtggatctgggacagatttcactctcaccatcagcagtctgcaacctgaagattttgctacgtactactgtcaacagcggcggtatttgcctatgacgttcggccaagggaccaaggtagaaatcaaacgg

SEQ ID NO: 190 - DNA, 324 bp, Homo sapiens
gacatccagatgacccagtctccatcctccctgtctgcatctgtaggagaccgtgtcaccatcacttgccgggcaagtcaggatattggggagtatttacagtggtatcagcagaaaccagggaaagcccctaagctcctgatctattggacttccatgttgcaaagtggggtcccatcacgtttcagtggcagtggatctgggacagatttcactctcaccatcagcagtctgcaacctgaagattttgctacgtactactgtcaacaggaggcgcggactccttttacgttcggccaagggaccaaggtggaaattaaacgg

SEQ ID NO: 191 - DNA, 324 bp, Homo sapiens
gacatccagatgacccagtctccatcctccctgtctgcatctgtaggagaccgtgtcaccatcacttgccgggcaagtcaggatattaatgattatttaagttggtaccagcagaaaccagggaaagcccctaagctcctgatctattgggggtcctctttgcaaagtggggtcccatcacgtttcagtggcagtggatctgggacagatttcactctcaccatcagcagtctgcaacctgaagattttgctacgtactactgtcaacagagggcgtataggccttatacgttcggccaagggaccaaggtggaaatcaaacgg

SEQ ID NO: 192 - DNA, 324 bp, Homo sapiens
gacatccagatgacccagtctccatcctccctgtctgcatctgtaggagaccgtgtcaccatcacttgccgggcaagtcaggatattgaggattggttagcttggtatcagcagaaaccagggaaagcccctaagctcctgatctattggggttccacgttgcaaagtggggtcccatcacgtttcagtggcagtggatctgggacagatttcactctcaccatcagcagtctgcaacctgaagattttgctacgtactactgtcaacagagtaagggtactccttatacgttcggccaagggaccaaggtggaaatcaaacgg

SEQ ID NO: 193 - DNA, 324 bp, Homo sapiens
gacatccagatgacccagtctccatcctccctgtctgcatctgtaggagaccgtgtcaccatcacttgccgggcaagtcaggatattgatgattggttacattggtaccagcagaaaccagggaaagcccctaagctcctgatctattggagttccagtttgcaaagtggggtcccatcacgtttcagtggcagtggatctgggacagatttcactctcaccatcagcagtctgcaacctgaagattttgctacgtactactgtcaacaggagaagtataggccttttacgttcggccaagggaccaaggtggaaatcaaacgg

SEQ ID NO: 194 - DNA, 324 bp, Homo sapiens
gacatccagatgacccagtctccatcctccctgtctgcatctgtaggagaccgtgtcaccatcacttgccgggcaagtcaggatattcagacttggttatcttggtatcagcagaaaccagggaaagcccctaaactcctgatctatcattcgtcctatttgcaaagtggggtcccatcacgtttcagtggcagtggatctgggacagatttcactctcaccatcagcagtctgcaacctgaagattttgctacgtactactgtcaacagtatgatacgttgcctggtacgttcggccaagggaccaaggtggaaatcaaacgg

SEQ ID NO: 195 - DNA, 324 bp, Homo sapiens
  misc_feature 83: n = A,T,C or G
gacatccagatgacccagtctccatcctccctgtctgcatctgtaggagaccgtgtcaccatcacttgccgggcgagtcagangatttcgggttgtttatattggtaccagcagaaaccagggaaagcccctaagctcctgatctatcgtggttcccatttgcaaagtggggtcccatcacgtttcagtggcagtggatctgggacagatttcactctcaccatcagcagtctgcaacctgaagattttgctacgtactactgtcaacaggattgtgatcctccttctacgttcggccaagggaccaaggtggaaatcaaacgg

SEQ ID NO: 196 - DNA, 324 bp, Homo sapiens
gacatccagatgacccagtctccatcctccctgtctgcatctgtaggagaccgtgtcaccatcacttgccgggcaagtcagtctattgagaagaagttagtttggtaccagcagaaaccagggaaagcccctaagctcctgatctattatacgtcctatttgcaaagtggggtcccatcacgtttcagtggcagtggatctgggacagatttcactctcaccatcagcagtctgcaacctgaagattttgctacgtactactgtcaacagtatcaggggcatcctctgacgttcggccaagggaccaaggtggaaatcaaacgg

SEQ ID NO: 197 - DNA, 334 bp, Homo sapiens
gacatccagatgacccagtctccatcctccctgtctgcatctgtaggagaccgtgtcaccatcacttgccgggcaagtcagcatattacggatcagttacggtggtaccagaagaaaccagggaaagcccctaagctcctgatctatagtgcttccattttgcaaagtggggtcccatcacgtttcagtggcagtggatctgggacagatttcactctcaccatcagcagtctgcaacctgaagattttgctacgtactactgtcaacagatttatattcggcctggtacgttcggccaagggaccaaggtggaaatcaaacgggcggccgcag

SEQ ID NO: 198 - DNA, 324 bp, Homo sapiens
gacatccagatgacccagtccccatcctccctgtctgcatctgtaggagaccgtgtcaccatcacttgccgggcaagtcagccgattggggatatgttaatgtggtaccagcagaaaccagggaaagcccctaagctcctgatctatggtgggtccaatttgcaaagtggggtcccatcacgtttcagtggcagtggatctgggacagatttcactctcaccatcagcagtctgcaacctgaagattttgctacgtactactgtcaacagcggcgtttggctcctagtacgttcggccaagggaccaaggtggaaatcaaacgg

SEQ ID NO: 199 - DNA, 324 bp, Homo sapiens
gacatccagatgacccagtctccatcctccctgtctgcatctgtaggagaccgtgtcaccatcacttgccgggcaagtcagcctattgatgagcgtctaaattggtaccagcagaaaccagggaaagcccctaagctcctgatctatcgtaggtcctggttgcaaagtggggtcccatcacgtttcagtggcagtggatctgggacagatttcactctcaccatcagcagtctgcaacctgaagattttgctacgtactactgtcaacagtgggggcatcatccttctacgttcggccaagggaccaaggtggaaatcaaacgg

SEQ ID NO: 200 - DNA, 324 bp, Homo sapiens
gacatccagatgacccagtctccatcctccctgtctgcatctgtaggagaccgtgtcaccatcacttgccgggcaagtcagcctattgattcgcgtttaatgtggtaccagcagaaaccagggaaagcccctaagctcctgatctattttgcttcctatttgcaaagtggggtcccatcacgtttcagtggcagtggatctgggacagatttcactctcaccatcagcagtctgcaacctgaagattttgctacgtactactgtcaacagtatcttatgcatcctcttacgttcggccaagggaccaaggtggaaatcaaacgg

SEQ ID NO: 201 - DNA, 324 bp, Homo sapiens
gacatccagatgacccagtctccatcctccctgtctgcatctgtaggagaccgtgtcaccatcacttgccgggcaagtcagccgattcattatgcgttagattggtaccagcagaaaccagggaaagcccctaagctcctgatctatagtacttccattttgcaaagtggggtcccatcacgtttcagcggcagtggatccgggacagatttcactctcaccatcagcagtctgcaacctgaagattttgctacgtactactgtcaacagtggtttaggtggcctactacgttcggccaagggaccaaggtggaaatcaaacgg

SEQ ID NO: 202 - DNA, 324 bp, Homo sapiens
gacatccagatgacccagtctccatcctccctgtctgcatctgtaggagaccgtgtcaccatcacttgccgggcaagtcagccgattggggattttttactgtggtaccagcagaaaccagggaaagcccctaagctcctgatctatggtgcttccacgttgcaaagtggggtcccatcacgtttcagtggcagtggatctgggacagatttcactctcaccatcagcagtctgcaacctgaagattttgctacgtactactgtcaacagaggcgtttttttccttctacgttcggccaagggaccaaggtggaaatcaaacgg

SEQ ID NO: 203 - DNA, 324 bp, Homo sapiens
gacatccagatgacccagtctccatcctccctgtctgcatctgtaggagaccgtgtcaccatcacttgccgggcaagtcagcatattggtcagaatttaaattggtatcagcagaaaccagggaaagcccctaagctcctgatctattgggggtccgatttgcaaagtggggtcccatcacgtttcagtggcagtggatctgggacagatttcactctcaccatcagcagtctgcaacctgaagattttgctacgtactactgtcaacagcttaggtttcctcctcttacgttcggccaagggaccaaggtggaaatcaaacgg

SEQ ID NO: 204 - DNA, 324 bp, Homo sapiens
gacatccagatgacccagtctccatcctccctgtctgcatctgtaggagaccgtgtcaccatcacttgccgggcaagtcaggatattggtgagtatttatattggtatcagcagaaaccagggaaagcccctaagctcctgatctatatgatttccaatttgcaaagtggggtcccatcacgtttcagtggcagtggatctgggacagatttcactctcaccatcagcagtctgcaacctgaagattttgctacgtactactgtcaacagttggtggcgtggccttatacgttcggccaagggaccaaggtggaaatcaaacgg

SEQ ID NO: 205 - DNA, 324 bp, Homo sapiens
gacatccagatgacccagtctccatcctccctgtctgcatctgtaggagaccgtgtcaccatcacttgccgggcaagtcaggatatttatggtgagttatcgtggtaccagcagaaaccagggaaagcccctaagctcctgatctattttagttccattttgcaaagtggggtcccatcacgtttcagtggcagtggatctgggacagatttcactctcaccatcagcagtctgcaacctgaagattttgctacgtactactgtcaacagaggtctgtgaggccttatacgttcggccaagggaccaaggtggaaatcaaacgg

SEQ ID NO: 206 - DNA, 324 bp, Homo sapiens
gacatccagatgacccagtctccatcctccctgtctgcatctgtaggagaccgtgtcaccatcacttgccgggcaagtcaggatattcatgggtatttagattggtatcagcagaaaccagggaaagcccctaagctcctgatctattatgcttcctatttgcaaagtggggtcccatcacgtttcagtggcagtggatctgggacagatttcactctcaccatcagcagtctgcaacctgaagattttgctacgtactactgtcaacagcggtatcagcatcctgttacgttcggccaagggaccaaggtggaaatcaaacgg

SEQ ID NO: 207 - DNA, 324 bp, Homo sapiens
gacatccagatgacccagtctccatcctccctgtctgcatctgtaggagaccgtgtcaccatcacttgccgggcaagtcaggagattgggcagtggttaaattggtaccagcagaaaccagggaaagcccctaagctcctgatctattggggttccgagttgcaaagtggggtcccatcacgtttcagtggcagtggatctgggacagatttcactctcaccatcagcagtctgcaacctgaagattttgctacgtactactgtcaacagacttctcgtaggccttttacgttcggccaagggaccaaggtggaaatcaaacgg

SEQ ID NO: 208 - DNA, 324 bp, Homo sapiens
gacatccagatgacccagtctccatcctccctgtctgcatctgtaggagaccgtgtcaccatcacttgccgggcaagtcaggatattaattcgcgtttaagttggtaccagcagaaaccagggaaagcccctaagctcctgatctattatgcgtcctatttgcgaagtggggtcccatcacgtttcagtggcagtggatctgggacagatttcactctcaccatcagcagtctgcaacctgaagattttgctacgtactactgtcaacagtggtggtcgcatcctattacgttcggccaagggaccaaggtggaaatcaaacgg

SEQ ID NO: 209 - DNA, 324 bp, Homo sapiens
gacatccagctgacccagtctccatcctccctgtctgcatctgtaggagaccgtgtcaccatcacttgccgggcaagtcaggatattggtgatcatttattgtggtatcagcagaaaccagggaaagcccctaagctcctgatctatggtgcttcccagttgcaaagtggggtcccatcacgtttcagtggcagtggatctgggacagatttcactctcaccatcagcagtctgcaacctgaagattttgctacgtactactgtcaacaggttcggatttatccccgtacgttcggccaagggaccaaggtggaaatcaaacgg

SEQ ID NO: 210 - DNA, 324 bp, Homo sapiens
gacatccagatgacccagtctccatcctccctgtctgcatctgtaggagaccgtgtcaccatcacttgccgggcaagtcaggatattgatcggtggttaaggtggtaccagcagaaaccagggaaagcccctaggctcctgatctattggacttccgagttgcaaagtggggtcccatcacgtttcagtggcagtggatctgggacagatttcactctcaccatcagcagtctgcaacctgaagattttgctacgtactactgtcaacaggagtttcggatgcctgtgacgttcggccaagggaccaaggtggaaatcaaacgg

SEQ ID NO: 211 - DNA, 324 bp, Homo sapiens
gacatccagatgacccagtctccatcctccctgtctgcatctgtaggagaccgtgtcaccattacttgccgggcaagtcaggatattggggatcatttattgtggtaccagcagaaaccagggaaagcccctaagctcctgatctatggtagttccgcgctgcaaagtggggtcccatcacgtttcagtggcagtggatctgggacagatttcactctcaccatcagcagtctgcaacctgaagattttgctacgtactactgtcaacaggttagggggtttccttcgacgttcggccaagggaccaaggtggaaatcaaacgg

SEQ ID NO: 212 - DNA, 324 bp, Homo sapiens
gacatccagatgacccagtctccatcctccctgtctgcatctgtaggagaccgtgtcaccatcacttgccgggcaagtcaggatattagtgattatttatcgtggtaccagcagaaaccagggaaagcccctaagctcctgatctattggacttccatgttgcaaagtggggtcccatcacgtttcagtggcagtagatctgggacagatttcactctcaccatcagcagtctgcaacctgaagattttgctacgtactactgtcaacagcgttatcgtcgtccttttacgttcggccaagggaccaaggtggaaatcaaacgg

SEQ ID NO: 213 - DNA, 324 bp, Homo sapiens
gacatccagatgacccagtctccatcctccctgtctgcatctgtaggagaccgtgtcaccatcacttgccgggcaagtcagtcgattgggaagcatttagcgtggtaccagcagaagccagggaaagcccctaagctcctgatctatagggcgtcccttttgcaaagtggggtcccatcacgtttcagtggcagtggatctgggacagatttcactctcaccatcagcagtctgcaacctgaagattttgctacgtactactgtcaacagcattctcggtcgcctaggacgttcggccaagggaccaaggtggaaatcaaacgg

SEQ ID NO: 214 - DNA, 324 bp, Homo sapiens
gacatccagatgacccagtctccatcctccctgtctgcatctgtaggagaccgtgtcaccatcacttgccgggcaagtcagtttattgggcttcatttagtgtggtaccagcagaaaccagggaaagcttccaagctcctgatctataatacgtccgatttgcaaagtggggtcccatcacgtttcagtggcagtggatctgggacagatttcaccctcaccatcagcagtctgcaacctgaagattttgctacgtactactgtcaacagatggctcattatccttatacgttcagccaagggaccaaggtggaaatcaaacgg

SEQ ID NO: 215 - DNA, 324 bp, Homo sapiens
gacatccagatgacccagtctccatcctccctgtctgcatctgtaggagaccgtgtcaccatcacttgccgggcaagtcagtctattggggatatgttactgtggtaccagcagaaaccagggaaagcccctaagctcctgatctatgggagttccgctttgcaaagcggggtcccatcacgtttcagtggcagtggatctgggacagatttcactctcaccatcagcagtctgcaacctgaagattttgctacgtactactgtcaacaggtgcggacgtatcctagtacgttcggccaagggaccaaggtggaaatcaaacgg

SEQ ID NO: 216 - DNA, 324 bp, Homo sapiens
gacatccagatgacccagtctccatcctccctgtctgcatctgtaggagaccgtgtcaccatcacttgccgggcaagtcagtatattgataagcgtttattgtggtatcagcagaaaccaggggaagcccctaagctcctgatctattatgcgtcctatttgcaaagtggggtcccatcacgtttcagtggcagtggatctgggacagatttcactctcaccatcagcagtctgcaacctgaagattttgctacgtactactgtcaacagcagtttattcatcctttgacgttcggccaagggaccaaggtggaaatcaaacgg

SEQ ID NO: 217 - DNA, 324 bp, Homo sapiens
gacatccagatgacccagtctccatcctccctgtctgcatctgtaggagaccgtgtcaccatcacttgccgggcaagtcagtatattggtcagatgttaaattggtaccagcagaaaccagggaaagcccctaagctcctgatctatcaggcttccgggttgcaaagtggggtcccatcacgtttcagtggcagtggatctgggacagatttcactctcacaatcagcagtctgcaacctgaagattttgctacgtactactgtcaacagtcttatgtgcatccttatacgttcggccaagggaccaaggtggaaatcaaacgg

SEQ ID NO: 218 - DNA, 324 bp, Homo sapiens
gacatccagatgacccagtctccatcctccctgtctgcatctgtaggagaccgtgtcaccatcacttgccgggcaagtcaggctattggtaattggttagattggtaccagcagaaaccagggaaagcccctaagctcctgatctattggggttccgagttgcaaagtggggtcccatcacgtttcagtggcagtggatctgggacagatttcactctcaccatcagcagtctgcaacctgaagattttgctacgtactactgtcaacagcgttcttcttcgccttttacgttcggccaagggaccaaggtggaaatcaaacgg

SEQ ID NO: 219 - DNA, 324 bp, Homo sapiens
gacatccagatgacccagtctccatcctccctgtctgcatctgtaggagaccgtgtcaccatcacttgccgggcaagtcaggctattgatatgtatttaacgtggtaccagcagaaaccagggaaagcccctaggctcctgatctattgggcttccatttcgcaaagtggggtcccatcacgtttcagtggcagtggatctgggacagattacactctcaccatcagcagtctgcaacctgaagattttgctacgtactactgtcaacagcgtaaggcgcggccttatacgttcggccaagggaccaaggtggaaatcaaacgg

SEQ ID NO: 220 - DNA, 324 bp, Homo sapiens
gacatccagatgacccagtctccatcctccctgtctgcatctgtaggagaccgtgtcaccatcacttgccgggcaagtcaggcgattgagtggtatttagcttggtaccagcagaaaccagggaaagcccctaagctcctgatctataatgcttccattttgcaaagtggggtcccatcacgtttcagtggcagtggatctgggacagatttcactctcaccatcagcagtctgcaacctgaagattttgctacgtactactgtcaacagagggcttttagtcctttgacgttcggccaagggaccaaggtggaaatcaaacgg

SEQ ID NO: 221 - DNA, 324 bp, Homo sapiens
gacatccagatgacccagtctccatcctccctgtctgcatctgtaggagaccgtgtcaccatcacctgccgggcaagtcaggctatttggacttatttaaattggtatcagcagaaaccagggaaagcccctaagctcctgatctatggtgcgtcccagttgcaaagtggggtcccatcacgcttcagtggcagtggatctgggacagatttcactctcaccatcagcagtctgcaacctgaagatttcgctacgtactactgtcaacagactgagagttttcctgttacgttcggccaagggaccaaggtggaaatcaaacgg

SEQ ID NO: 222 - DNA, 324 bp, Homo sapiens
gacatccagatgacccagtctccatcctccctgtctgcatctgtaggagaccgtgtcaccatcacttgccgggcaagtcagacgattactgattatttaaattggtatcagcagaaaccagggaaagcccctaagctcctgatctattgggggtccattttgcaaagtggggtcccatcacgtttcagtggcagtggatctgggacagatttcactctcaccatcagcagtctgcaacctgaagattttgctacgtactactgtcaacagtctgcgcataggccttatacgttcggccaagggaccaaggtggaaatcaaacgg

SEQ ID NO: 223 - DNA, 324 bp, Homo sapiens
gacatccagatgacccagtctccatcctccctgtctgcatctgtaggagaccgtgtcaccatcacttgccgggcaagtcagcagattgatgataggttatcttggtaccagcagaaaccagggaaagcccctaagctcctgatctattttaagtcctttttgcaaagtggggtcccatcacgtttcagtggcagtggatctgggacagatttcactctcaccatcagcagtctacaacctgaagattttgctacgtactactgtcaacagtatcaggcgcatcctttgacgttcggccaagggaccaaggtggaaatcaaacgg

SEQ ID NO: 224 - DNA, 324 bp, Homo sapiens
gacatccagatgacccagtctccatcctccctgtctgcatctgtaggagaccgtgtcaccatcacttgccgggcaagtcagaggattgctggttgtttatcttggtatcagcagaaaccagggaaagcccctaagctcctgatctatcgtacttccttgttgcaaagtggggtcccatcacgtttcagtggcagtggatctgggacagatttcactctcaccatcagcagtctgcaacctgaagattttgctacgtactactgtcaacaggattgtacgtttcctaggacgttcggccaagggaccaaggtggaaatcaaacgg

SEQ ID NO: 225 - DNA, 324 bp, Homo sapiens
gacatccagatgacccagtctccatcctccctgtctgcatctgtaggagaccgtgtcaccatcacttgccgggcgagtcagaggatttcgggttgtttatattggtaccagcagaaaccagggaaagcccctaagctcctgatctatcgtggttcccatttgcaaagtggggtcccatcacgtttcagtggcagtggatctgggacagatttcactctcaccatcagcagtctgcaacctgaagattttgctacgtactactgtcaacaggattgtgatcctccttctacgttcggccaagggaccaaggtggaaatcaaacgg

SEQ ID NO: 226 - DNA, 324 bp, Homo sapiens; identical to SEQ ID NO: 217

SEQ ID NO: 227 - DNA, 324 bp, Homo sapiens
gacatccagatgacccagtctccatcctccctgtctgcatctgtaggagaccgtgtcaccatcacctgccgggcaagtcagaatattggttcgcacttattgtggtatcagcagaaaccagggaaagcccctaagctcctgatctatggctcttccagtttgcaaagtggggtcccatcacgtttcagtggcagtggatctgggacggatttcactctcaccatcagcagtctgcaacccgaagattttgctacgtactactgtcaacaggttaggctggctcctcatacgttcggccaagggaccaaggtggaaatcaaacgg

SEQ ID NO: 228 - DNA, 324 bp, Homo sapiens
gacatccagatgacccagtctccatcctccctgtctgcatctgtaggagaccgtgtcaccatcacttgccgggcaagtcagaatattgggatgtatttaaagtggtatcagcagaaaccagggaaagcccctaagctcctgatctattattcttccagtttgcaaagtggggtcccatcacgtttcagtggcagtggatctgggacagatttcactctcaccatcagcagtctgcaacctgaagattttgctacgtactactgtcaacagaatcgtatgcggcctactacgttcggccaagggaccaaggtggaaatcaaacgg

SEQ ID NO: 229 - DNA, 324 bp, Homo sapiens
gacatccagatgacccagtctccatcctccctgtctgcatctgtaggagaccgtgtcaccatcacttgccgggcaagtcagggtattgattggtatttatcttggtatcagcagaaaccagggaaagcccctaagctcctgatctatgagggttccaatttgcaaagtggggtcccatcacgtttcagtggcagtggatctggaacagatttcactctcaccatcagcagtctgcaacctgaagattctgctacgtactactgtcaacagagggctgcttatccttttacgttcggccaagggaccaaggtggaaatcaaacgg

SEQ ID NO: 230 - DNA, 324 bp, Homo sapiens
gacatccagatgacccagtctccatcctccctgtctgcatctgtaggagaccgtgtcaccatcacttgccgggcaagtcaggggattggggttgcgttagattggtatcagcagaaaccagggaaagcccctaagctcctgatctatatggcttccaggttgcaaagtggggtcccatcacgtttcagtggcagtggatctgggacagatttcactctcaccatcagcagtctgcaacctgaagattttgctacgtactactgtcaacagtattcggagcttcctgttacgttcggccaagggaccaaggtggagatcaaacgg

SEQ ID NO: 231 - DNA, 324 bp, Homo sapiens
gacatccagatgacccagtctccatcctccctgtctgcatctgtaggagaccgtgtcaccatcacttgccgggcaagtcaggagattgggcagtggttaaattggtaccagcagaaaccagggaaagcccctaagctcctgatctattggggttccgagttgcaaagtggggtcccatcacgtttcagtggcagtggatctgggacagatttcactctcaccatcagcagtctgcaacctgaagattttgctacgtactactgtcaacagactcagcttaggcctagtacgttcggccaagggaccaaggtggaaatcaaacgg

SEQ ID NO: 232 - protein, 108 aa, Homo sapiens
DIQMTQSPSSLSASVGDRVTITCRASQSIRRYLTWYQQKPGKAPKLLIYSASHLQSGVPLRFSGSGFGTDFTLTISSLQPEDFATYYCQQAYIAPFTFGQGTKVEIKR

SEQ ID NO: 233 - protein, 108 aa, Homo sapiens
DIQMTQSPSSLSASVGDRVTITCRASQEIHDYLSWYQQKPGKAPKLLIYLSSRLQSGVSSRFSGSGSGTDFTLTISSLQPEDFATYYCQQYYGYPYTFGQGTKVEIKR

SEQ ID NO: 234 - protein, 107 aa, Homo sapiens
DIQMTQSPSSLSASVGDRVTITCRASQNIDSRLSWYQQKPGKAPKLLIYRTSVLQSGVPSRFSGSGSGTDFTLTISSLQPEDFATYYCQQWDMFPLTFGQGTKVEVK

SEQ ID NO: 235 - protein, 108 aa, Homo sapiens
DIQMTQSPSSLSASVGDRVTITCRASQPILFSLNWYQQKPGKAPKLLIYSASSLQSGVSSRFSGSGFGTDFTLTISSLQPEDFATYYCQQHHSRPYTFGQGTKVEIKR

SEQ ID NO: 236 - protein, 108 aa, Homo sapiens
DIQMTQSPSSLSASVGDRVTITCRASQMIRNYLLWYQQAPGKAPKLLIYNASKLQSGVPSRFSGSGSGTDFTLTISSLQPEDFATYYCQQAHTAPFTFGQGTKVEIKR

SEQ ID NO: 237 - protein, 108 aa, Homo sapiens
DIQMTQSPSSLSASVGDRVTITCRASQRISHALRWYQQKPGKAPKLLIYRASALQSGVPSRFSGSGSGTDFTLTISSLQPEDFATYYCQQNRSVPFTFGQGTKVEIKR

SEQ ID NO: 238 - protein, 108 aa, Homo sapiens
DIQMTQSPSSLSASVGDRVTITCRASQYINTLLSWYQQKPGKAPKLLIYAQSRLQSGVPSRFSGSGSGTDFTLTISSLQPEDFATYYCQQFAFRPYTFGQGTKVEIKR

SEQ ID NO: 239 - protein, 108 aa, Homo sapiens
DIQMTQSPSSLSASVGDRVTITCRASQNIRRYLVWYQQKPGKAPKLLIYNASHLQSGVPSRFSGSGSGTDFTLTISSLQPEDFATYYCQQIYLSPFTFGQGTKVEIKR

SEQ ID NO: 240 - protein, 108 aa, Homo sapiens
DIQMTQSPSSLSASVGDRVTITCRASQSIGRYIYWYQQKPGKAPKLLIYNVSYLQSGVPSRFSGSGSGTDFTLTISSLQPEDFATYYCQQCFRGPCTFGQGTKVEIKR

SEQ ID NO: 241 - protein, 108 aa, Homo sapiens
DIQMTQSPSSLSASVGDRVTITCRASQRIGRYLNWYQQKPGKAPELLIYWVSRLQSGVPSRFSGSGFGTDFTLTISSLQPEDFATYYCQQVHSFPMTFGQGTKVEIKR

SEQ ID NO: 242 - protein, 108 aa, Homo sapiens
DIQMTQSPSSLSASVGDRVTITCRASTEIGRRLLWYQQKPGKAPKLLISAGSVLQSGVPSRFSGSGSGTDFTLTISSLQPEDFATYYCGQYHERPETFGQGTKVEIKR

SEQ ID NO: 243 - protein, 108 aa, Homo sapiens
DIQMTQSPSSLSASVGDRVTITCRASSPINWSLNWYQQKPGKAPKLLIGLGSVLQSGVPSRFSGSGSGTDFTLTISSLQPEDFATYYCAQSGRGPETFGQGTKVEIKR

SEQ ID NO: 244 - protein, 108 aa, Homo sapiens
DIQMTQSPSSLSASVGDRVTITCRASQYIGRHLVWYQQKPGKAPKLLIYFASMLQSGVPSRFSGSGSGTDFTLTISSLQPEDFATYHCQQVHFDPFTFGQGTKVEIKR

SEQ ID NO: 245 - protein, 108 aa, Homo sapiens
DIQMTQSPSSLSASVGDRVTITCRASQPIHDYLTWYQQKPGKAPKLLIYLASRLQSGVPSRFSGSGSGTDFTLTISSLQPEDFATYYCQQYHVLPYTFGQGTKVEIKR

SEQ ID NO: 246 - protein, 108 aa, Homo sapiens
DIQMTQSPSSLSASVGDRVTITCRASQEIHDYLSWYQQKPGKAPKLLIYLSSRLQSGVPSRFSGSRSGTDFTLTISSLQPEDFATYYCQQYHKYPYTFGQGTKVEIKR

SEQ ID NO: 247 - protein, 108 aa, Homo sapiens
DIQMTQSPSSLSASVGDRVTITCRASQSINVRLIWYQQKPGKDPKLLIYSSSHLQSGVPSRFSGSGSGTDFTLTISSLQPEDFATYYCQQYHYTPFTFGQGTKVEIKR

SEQ ID NO: 248 - protein, 108 aa, Homo sapiens
DIQMTQSPSSLSASVGDRVTITCRASQEIHDYLSWYQQKPGKAPKLLIYLSSRLQSGVPSRFSGSGSGTDFTLTISSLQPEDFATYYCQQYHKGPYTFGQGTKVEIKR

SEQ ID NO: 249 - protein, 108 aa, Homo sapiens
DIQMTQSPSSLSASVGDRVTITCRASQFIGRYLYWYQQKPGKAPKLLIYDTSALQSGVPSRFSGSGSGTDFTLTISSLQPEDFATYYCQQAYLGPATFGQGTKVEIKR

SEQ ID NO: 250 - protein, 108 aa, Homo sapiens
DIQMTQSPSSLSASVGDRVTITCRASQSISRYLNWYQQKPGKAPKLLIYMISRLQSGVPSRFSGSGSGTDFTLTISSLQPEDFATYYCQQNYLAPDTFGQGTKVEIKR

SEQ ID NO: 251 - protein, 108 aa, Homo sapiens
DIQMTQSPSSLSASVGDRVTITCRASQNILWSLSWYQQKPGKAPKLLIYQASQLQSGVPSRFSGSGFGTDFTLTISSLQPEDFATYYCQQRHSPPHTFGQGTKVEIKR

SEQ ID NO: 252 - protein, 108 aa, Homo sapiens; identical to SEQ ID NO: 249

SEQ ID NO: 253 - protein, 108 aa, Homo sapiens; identical to SEQ ID NO: 247

SEQ ID NO: 254 - protein, 108 aa, Homo sapiens
DIQITQSPSSLSASVGDRITITCRASQKIENDLAWYQQKPGKAPKLLIYYTSILQSGVPSRFSGSGSGTDFTLTISSLQPEDFATYYCQQRRYVPATFGQGTKVEIKR

SEQ ID NO: 255 - protein, 108 aa, Homo sapiens
DIQMTQSPSSLSASVGDRVTITCRASQYIGIDLQWYQQKPGKAPELLIYRGSFLHSGVPSRFSGSGSGTDFTLTISSLQPEDFATYYCQQRWIRPHTFGQGTKVEIKR

SEQ ID NO: 256 - protein, 108 aa, Homo sapiens
DIQMTQSPSSLSASVGDRVTITCRASQWIGDSLVWYQQKPGKAPKLLIYFGSILQSGVPSRFSGSGSGTDFTLTISSLQPEDFATYYCQQHKTSPSTFGQGTKVEIKR

SEQ ID NO: 257 - protein, 108 aa, Homo sapiens
DIQMTQSPSSLSASVGDRVTITCRASQRISTYLNWYQQKPGKAPKLLIYRSSMLQSGVPSRFSGSGSGTDFTLTISSLQPEDFATYYCQQYSFSPLTFGQGTKVEIKR

SEQ ID NO: 258 - protein, 108 aa, Homo sapiens
DIQMTQSPSSLSASVGDRVTITCRASQNIKRYLYWYQQKPGKAPKLLIYHISTLQSGVPSRFSGSGSGTDFTLTISSLQPEDFATYYCQQSFRAPITFGQGTKVEIKR

SEQ ID NO: 259 - protein, 108 aa, Homo sapiens
DIQMTQSPSSLSASVGDRVTITCRASQHIGSMLEWYQQKPGKAPKLLIYRASFLQSGVPSRFSGSGSGTDFTLTISSLQPEDFATYYCQQGRALPFTFGQGTKVEIKR

SEQ ID NO: 260 - protein, 108 aa, Homo sapiens
DIQMTQSPSSLSASVGDRVTITCRASQVIGSRLIWYQQKPGKDPKLLIYRASRLQSGVPSRFSGSGSGTDFTLTISSLQPEDFATYYCQQYKLDPYTFGQGTKVEIKR

SEQ ID NO: 261 - protein, 108 aa, Homo sapiens
DIQMTQSPSSLSAFVGDRVTITCRASQDIEGQLRWYQQKPGKAPKLLIYFGSLLQSGVPSRFSGSGSGTVYTLTISSLQPEDFATYYCQQSHLFPATFGQGTKVEIKR

SEQ ID NO: 262 - protein, 108 aa, Homo sapiens
DIQVTQSPSSLSASVGDRVTITCRASQEISSCLNWYQQKPGKAPKLLIYCTSVLQSGVPSRFSGSGSGTDFTLTISSLQPEDFATYYCQQRFGNPLTFGQGTKVEIKR

SEQ ID NO: 263 - protein, 108 aa, Homo sapiens
DIQMTQSPSSLSASVGDRVTITCRASQPIDGNLRWYQQKPGKAPRLLIYFTSILQSGVPSRFSGSGSGTDFTLTISSLQPEDFATYYCQQHRHWPATFGQGTKVEIKR

SEQ ID NO: 264 - protein, 108 aa, Homo sapiens
DIQMTQSPSSLSASVGDRVTITCRASQDIGTALLWYQQKPGKDPRLLIYRGSHLQSGVPSRFSGSGSGTDFTLTISSLQPEDFATYSCQQYRYEPMTFGQGTKVEIKR

SEQ ID NO: 265 - protein, 108 aa, Homo sapiens
DIQMTQSPSSLSASVGDRVTITCRASQPIQGWLNWYQQKPGKAPKLLIYYSSLLQSGVPSRFRGSGSGTDFTLTISSLQPEDFATYYCQQREVKPFTFGQGTKVEIKR

SEQ ID NO: 266 - protein, 108 aa, Homo sapiens
DIQMTQSPSSLSASVGDRVTITCRASQNIAWQLRWYQQKPGKAPTLLIYATSQLQSGVPSRFSGSGSGTDFTLTISSLQPEDFATYYCQQTASFPVTFGQGTKVEIKR

SEQ ID NO: 267 - protein, 108 aa, Homo sapiens
DIQMTQSPSSLSASVGDRVTITCRASQEIHDYLSWYQQKPGKAPKLLIYLSSRLQSGVPSRFSGSRSGTDFTLTISSLQPEDFATYYCQQYHLGPYTFGQGTKVEIKR

SEQ ID NO: 268 - protein, 108 aa, Homo sapiens
DIQMTQSPPSLSASVGDRVTITCRASQHIYNALRWYQQKPGKAPKLLIYASSKLQSGVPSRFSGSGSGTDFTLTISSLQPEDFATYYCQQHYSTPYTFGQGTKVEIKR

SEQ ID NO: 269 - protein, 108 aa, Homo sapiens
DIQMTQSPSSLSASVGDRVTITCRASHNIDSRLSWYQQKPGKAPKLLIYRASVLQSGVPSRFRGSGSGTDFTLTISSLQPEDFATYYCQQWDMFPLSFGQGTKVEIKR

SEQ ID NO: 270 - protein, 108 aa, Homo sapiens
DIQMTQSPSSLSASVGDRVTITCRASQNIDSRLSWYQQKPGKAPKLLIYRTSVLQSGVPSRFSGSGSGTDFTLTISSLQPEDFATYYCQQWDMFPLMFGQGTKVEIKR

SEQ ID NO: 271 - protein, 108 aa, Homo sapiens
DIQMTQSPSSLSASVGDRVTITCRASQNIDSRLSWYQQKPGKAPKLLIYRTSVLQSGVPSRFSGSGSGTVFTLTISSLQPEDFATYYCQQWDMFPLAFGKGTKVEIKR

SEQ ID NO: 272 - protein, 108 aa, Homo sapiens
DIQVTQSPSSLSASVGDRVTITCRASQNIDSRLSWYQQKPGKAPKLLIYRATVLQSGVPSRFSGSGSGTDFTLTISSLQPEDFATYYCQQWDMFPLTFGQGTKVEIKR

SEQ ID NO: 273 - protein, 108 aa, Homo sapiens
DIQMTQSPSSLSASVGDRVTITCRASQNIDSRLSWYQQKPGKAPKLLIYRASVLQSGVPSRFSGSGSGTDFTLTISSLQPEDFATYYCQQWDMFPLSFGHGTKVEIKR

SEQ ID NO: 274 - protein, 108 aa, Homo sapiens
DIQMTQSPSSLSASVGDRVTITCRASQNIDSRLSWYQQKPGKAPKLLIYRTSVLQSGVPTRFSGSGSGTDFTLTISSLQPEDFATYYCQQWDMFPLTFGQGTKVEIKR

SEQ ID NO: 275 - protein, 108 aa, Homo sapiens; identical to SEQ ID NO: 270

SEQ ID NO: 276 - protein, 108 aa, Homo sapiens
DIQMTQSPSSLSASVGDRVTITCRASQNIDSRLSWYQQKPGKAPKLLVYRASVLQSGVPSRFSGSGSGTDFTLTISSLQPEDFATYHCQQWDMFPLTLGQGTKVEIKR

SEQ ID NO: 277 - protein, 108 aa, Homo sapiens
DIQMTQSPSSLSASVGDRVTITCRASQNIDSRLSWYQQKPGKAPKLLIYRASVLQSGVPSRFSGSGSGTDFTLTISSLQPEDFATYYCQQWDMFPLAFGQGTKVEIKR

SEQ ID NO: 278 - protein, 108 aa, Homo sapiens
DIQMTQSPSSLSASVGDRVTITCRASQNIDSRLSWYQQKPGKAPKLLIYRASVLQSGVPSRFSGTGSGTDFTLTISSLQPEDFATYYCQQWDMFPLSFGQGTKVEIKR

SEQ ID NO: 279 - protein, 108 aa, Homo sapiens
DIQMTQSPSSLSASVGDRVTITCRASQNIDSRLSWYQEKPGKAPKLLIYRASVLQSGVSSRFSGSGSGTDFTLTISSLQPEDFATYYCQQWDMFPLTFGRGTKVEIKR

SEQ ID NO: 280 - protein, 108 aa, Homo sapiens
DIQMTQSPSSLSASVGDRVTITCRASQNIDSRLSWYQQKPGKDPKLLIYRSSVLQSGVPSRFSGSGSGTDFTLTISSLQPEDFATYYCQQWDMFPLTFGQGTKVEIKR

SEQ ID NO: 281 - protein, 108 aa, Homo sapiens
DIQTTQSPSSLSASVGDRVTITCRASQNIDSRLSWYQQKPGKAPKLLIYRSSVLQSGVPSRFSGSGSGTDFTLTISSLQPEDFATYYCQQWDMFPLMFGQGTKVEIKR

SEQ ID NO: 282 - protein, 108 aa, Homo sapiens
DIQMTQSPSSLSASVGDRVTITCRASQNIDSRLSWYQQKPGKAPKLLIYRSSILQSGVPSRFSGSGSGTDFTLTISSLQPEDFATYHCQQWDMFPLTFGQGTKVEIKR

SEQ ID NO: 283 - protein, 108 aa, Homo sapiens
DIQMTQSPSSLSASVGDRVTITCRASQNIDSRLSWYQQKPGKDPKLLIYRASVLQSGVPSRFSGSGSGTDFTLTISSLQPEDFATYYCQQWDMFPLTFSQGTKVEIKR

SEQ ID NO: 284 - protein, 108 aa, Homo sapiens
DIQMTQSPSSLSASVGDRVTITCRASQNIDSRLSWYQQKPGKAPKLLIYRASVLQSGVPSRFSGSGSGTDFTLTISSLQPEDFATYYCQQWDMFPLAFGQGTRVEIKR

SEQ ID NO: 285 - protein, 108 aa, Homo sapiens
DIQMTQSPSSLSASVGDRVTITCRASRNIDSRLSWYQQKPGKAPKLLIYRTSVLQSGVPSRFSGSGSGTDFTLTISSLQPEDFATYYCQQWDMFPLMFGQGTKVEIKR

SEQ ID NO: 286 - protein, 108 aa, Homo sapiens
DIQMTQSPSSLSASVGDRVTITCRASQNIDSRLSWYQEKPGKAPKLLIYRTSVLQSGVPSRFSGSGSGTDFTLTISSLQPEDFATYYCQQWDMFPLTFGQGTRVEIKR

SEQ ID NO: 287 - protein, 108 aa, Homo sapiens
DIQMTQSPSSLSASVGDRVTITCRASQNIDSRLSWYQQKPGKAPKLLIYRASVLQSGVPSRFSGSGSGTDFTLTISSLQPEDFATYHCQQWDMFPLTFGQGTKVEIKR

SEQ ID NO: 288 - protein, 108 aa, Homo sapiens
DIQMTQSPSSLSASVGDRVTITCRASQNIDSRLSWYQQKPGKAPKLLIYRTSVLQSGVPSRFSGSGSGTEFTLTISSLQPEDFATYYCQRWDMFPLTFGQGTKVEIKR

SEQ ID NO: 289 - protein, 108 aa, Homo sapiens
DIQMTQSPSSLSASVGDRVTITCRASQNIDSRLSWYQQKPGKAPKLLIYRTSVLQSGVPSRFSGSGSGTDFTLTISSLQPEDFATYYCQQWDMFPLTFGHGTKVEIKR

SEQ ID NO: 290 - protein, 108 aa, Homo sapiens
DIQMTQSPSSLSASVGDRVTITCRASLKIENDLAWYQQKPGKAPKLLIYYTSILQSGVPSRFSGSGSGTDFTLTISSLQPEDFATYYCQQRRYAPATFGQGTKVEIKR

SEQ ID NO: 291 - protein, 108 aa, Homo sapiens
DIQMTQSPSSLSASVGDRVTITCRTSQKIENDLAWYQQKPGKAPKLLIYYTSILQSGVPSRFSGSGSGTDFTLTISSLQPEDFATYYCQQRRYVPATFGQGTKVEIKR

SEQ ID NO: 292 - protein, 108 aa, Homo sapiens
DIQMTQSPSSLSASVGDRVTITCRASQKIENDLAWYQQKPGNAPKLLIYYTSILHSGVPSRFSGSGSGTDFTLTISSLQPEDFATYYCQQRRYVPATFGQGTKVEIKR

SEQ ID NO: 293 - protein, 108 aa, Homo sapiens
DIQMTQSPSSLSASVGDRVTITCRTSQKIENDLAWYQQRPGKAPKLLIYYTSILQSGVPSRFSGSGSGTDFTLTISSLQPEDFATYYCQQRRYVPATFGQGTKVEIKR

SEQ ID NO: 294 - protein, 108 aa, Homo sapiens
DIQMTQSPSSLSASVGDRVTITCRASQKIENDLAWYQQKPGKAPKLLIYYTSILQSGIPSRFSGSGSGTDFTLTISSLQPEDFATYYCQQRRYAPATFGQGTKVEIKR

SEQ ID NO: 295 - protein, 108 aa, Homo sapiens
DIQMTQSPSSLSASVGDRVTITCRASQKIENDLAWYQQKPGKAPKLLIYYTSILQSGVPSRFSGSGSGTDFTLTISSLQPEDFATYYCQQRRYAPATFGQGTKVEIKR

SEQ ID NO: 296 - protein, 108 aa, Homo sapiens
DIQMTQSPSSLSASVGDRVTITCRASQKIENDLAWYQQKPGKAPKLLIYYTSILQSGVPSRFSGSGSGTDFTLTISSLQPEDFATYYCQQRRYVPASFGQGTKVEIKR

SEQ ID NO: 297 - protein, 108 aa, Homo sapiens; identical to SEQ ID NO: 295

SEQ ID NO: 298 - protein, 108 aa, Homo sapiens
DIQMTQSPSSLSASVGDRVTITCRASQKIENDLAWYQQKPGKAPKLLIYYTSILQRGVPSRFSGSGSGTDFTLTISSLQPEDFATYYCQQRRYAPATFGQGTKVEIKR

SEQ ID NO: 299 - protein, 108 aa, Homo sapiens
DIQMTQSPSSLSASVGDRVTITCRTSQKIENDLAWYQQKPGKAPKLLIYYTSILQSGVPSRFSGSGSGTDFTLTISSLQPEDFATYYCQQRRYAPATFGQGTKVEIKR

SEQ ID NO: 300 - protein, 108 aa, Homo sapiens
DIQMTQSPSSLSASVGDRVTITCRANQKIENDLAWYQQKPGKAPKLLIYYTSILQSGVPSRFRGSGSGTDFTLTISSLQPEDFATYYCQQRRYVPATFGQGTKVEIKR

SEQ ID NO: 301 - protein, 108 aa, Homo sapiens
DIQMTQSPSSLSASVGDRVTITCRASQKIENDLAWYQQKPGKAPKLLIYYTSILHSGVPSRFSGSGSGTDFTLTISSLQPEDFATYYCQQRRYVPATFGQGTKVEIKR

SEQ ID NO: 302 - protein, 108 aa, Homo sapiens
DIQMTQSPSSLSASVGDRVTITCQASKKIENDLAWYQQKPGKAPKLLIYYTSILQSGVPSRFSGSGSGTDFTLTISSLQPEDFATYYCQQRRYVPATFGQGTKVEIKR

SEQ ID NO: 303 - protein, 108 aa, Homo sapiens
DIQLTQSPSSLSASVGDRVTITCRASQKIENDLAWYQQKPGKAPKLLIYYTSILQRGVPSRFSGSGSGTDFTLTISSLQPEDIATYYCQQRRYVPATFGQGTKVEIKR

SEQ ID NO: 304 - protein, 108 aa, Homo sapiens
DIQMTQSPSSLSASVGDRVTITCRASQKIENDLAWYQQKPGKAPKLLIYYTSILQSGVPSRFIGSGSGTDFTLTISSLQPEDFATYYCQQRRYVPATFGPGTKVEIKR

SEQ ID NO: 305 - protein, 108 aa, Homo sapiens
DIQMTQAPSSLSASVGDRVTITCRSSQKIENDLAWYQQKPGKAPKLLIYYTSILQSGVPSRFSGSGSGTDFTLTISSLQPEDFATYYCQQRRYVPATFGQGTKVEIKR

SEQ ID NO: 306 - protein, 108 aa, Homo sapiens
DIQMTQSPSSLSASVGDRVTITCRASQNIESWLRWYQQKPGKAPKLLIYHSSRLQSGVPSRFSGSGSGTDFTLTISSLQPEDFATYYCQQSRVRPFTFGQGTKVEIKR

SEQ ID NO: 307 - protein, 108 aa, Homo sapiens
DIQMTQSPSSLSASVGDRVTITCRASQSISKNLLWYQQKPGKAPKLLIYHSSFLQSGVPSRFSGSGSGTDFTLTISSLQPEDFATYYCQQRFRYPHTFGQGTKVEIKR

SEQ ID NO: 308 - protein, 108 aa, Homo sapiens
DIQMTQSPSSLSASVGDRVTITCRASQHIGRRLLWYQQKPGKAPKLLIYYSSKLQSGVPSRFSGSGSGTDFTLTISSLQPEDFATYYCQQRTIQPHTFGQGTKVEIKR

SEQ ID NO: 309 - protein, 108 aa, Homo sapiens
DIQMTQSPSSLSASVGDRVTITCRASQWINDRLSWYQQKPGKAPKLLIYRKSGLQSGVPSRFSGSGSGTDFTLTISSLQPEDFATYYCQQFRNIPFTFGQGTKVEIKR

SEQ ID NO: 310 - protein, 108 aa, Homo sapiens
DIQMTQSPSSLSASVGDRVTITCRASQSIGRRLNWYQQKPGKAPKLLIYRTSTLQSGVPSRFSGSGSGTDFTLTISSLQPEDFATYYCQQTSRVPYTFGQGTKVEIKR

SEQ ID NO: 311 - protein, 108 aa, Homo sapiens
DIQMTQSPSSLSASVGDRVTITCRASQNIDRRLLWYQQKPGKAPKLLIYGSSKLQSGVPSRFSGSRSGTDFTLTISSLQPEDFATYYCQQRIYDPHTFGQGTKVEIKR

SEQ ID NO: 312 - protein, 108 aa, Homo sapiens
DIQMTQSPSSLSASVGDRVTITCRASQDIGRNLLWYQQKPGKAPRLLIYYSSRLQSGVPSRFSGSGSGTDFTLTISSLQPEDLATYYCQQRSRNPFTFGQGTKVEIKR

SEQ ID NO: 313 - protein, 108 aa, Homo sapiens
DIQMTQSPSSLSASVGDRVTITCRASQDIKKHLLWYQQRPGKAPKLLIYYSSRLQSGVPSRFSGSGSGTDFTLTISSLQPEDFATYYCQQRHHDPFTFGQGTKVEIKR

SEQ ID NO: 314 - protein, 108 aa, Homo sapiens
DIQMTQSPSSLSASVGDRVTITCRASQSIDRRLLWYQQKPGKAPKLLIYRASRLQSGVPSRFSGSGSGTDFTLTISSLQPEDFATYYCQQTYARPNTFGQGTKVEIKR

SEQ ID NO: 315 - protein, 108 aa, Homo sapiens
DIQMTQSPSSLSASVGDRVTITCRASQDIGGRLHWYQQKPGKAPKLLIYQASKLQSGVPSRFSGSGSGTDFTLTISSLQPEDFATYYCQQKRRQPHTFGQGTKVEIKR

SEQ ID NO: 316 - protein, 108 aa, Homo sapiens
DIQMTQSPSSLSASVGDRVTITCRASQSIDRRLGWYQQKPGKAPKLLISGSSRLQSGVPSRFSGSGSGTDFTLTISSLQPEDFATYYCVQRQRLPYTFGQGTKVEIKR

SEQ ID NO: 317 - protein, 108 aa, Homo sapiens
DIQMTQSPSSLSASVGDRVTITCRASQSISSYLNWYQQKPGKAPKLLIRRGSLLQSGVPSRFSGSGSGTDFTLTISSLQPEDFATYYCRQGMARPWTFGQGTKVEIKR

SEQ ID NO: 318 - protein, 108 aa, Homo sapiens
DIQMTQSPSSLSASVGDRVTITCRASQSIGNNLLWYQQKPGKAPKLLIYYTSRLQSGVPSRFSGSGSGTDFTLTISSLQPEDFATYYCQQRRTHPHTFGQGTKVEIKR

SEQ ID NO: 319 - protein, 108 aa, Homo sapiens
DIQMTQSPSSLSASVGDRVTITCRASQLINRRLSWYQQKPGKPPKLLIYHHSRLQSGVPSRFSGSGSGTDFTLTISSLQPEDFATYYCQQTRIRPHTFGQGTKVEIKR

SEQ ID NO: 320 - protein, 108 aa, Homo sapiens
DIQMTQSPSSLSASVGDRVTITCRASQNIDRRLLWYQQKPGKAPKLLIYGSSKLQSGVPSRFSGSGSGTDFTLTISSLQPEDFATYYCQQRIYDPHTFGQGTKVEIKR

SEQ ID NO: 321 - protein, 108 aa, Homo sapiens
DIQMTQSPSSLSASVGDRVTITCRASQTIRKRLHWYQQKPGKAPKLLIYHASKLQSGVPSRFSGSGSGTDFTLTISSLQPEDFATYYCQQRSDPPYTFGQGTKVEIKR

SEQ ID NO: 322 - protein, 108 aa, Homo sapiens
DIQMTQSPSSLSASVGDRVTITCRASQEIRKRLRWYQQKPGKAPKLLIYRASTLQSGVPSRFSGSGSGTDFTLTISSLQPEDFATYYCQQLFQSPWTFGQGTKVEIKR

SEQ ID NO: 323 - protein, 108 aa, Homo sapiens
DIQMTQSPSSLSASVGDRVTITCRASQEIHKRLLWYQQKPGKAPKLLIYSGSTLQSGVPSRFSGSGSGTDFTLTISSLQPEDFATYYCQQRYLQPHTFGQGTKVEIKR

SEQ ID NO: 324 - protein, 108 aa, Homo sapiens; identical to SEQ ID NO: 315

SEQ ID NO: 325 - protein, 108 aa, Homo sapiens; identical to SEQ ID NO: 315

SEQ ID NO: 326 - protein, 108 aa, Homo sapiens
DIQMTQSPSSLSASVGDRVTITCRASQEIDRRLLWYQQKPGKAPKLLIYSASRLQSGVPSRFSGSGSGTDFTLTISSLQPEDFATYYCQQRYHMPHTFGQGTKVKIKR

SEQ ID NO: 327 - protein, 108 aa, Homo sapiens
DIQMTQSPSSLSASVGDRVTITCRASQDIFTKLRWYQQKPGKAPRLLIYAGSRLQSGVPSRFSGSGSGTDFTLTISSLQPEDFATYYCQQVKQKPWTFGQGTKVEIKR

SEQ ID NO: 328 - protein, 108 aa, Homo sapiens
DIQMTQSPSSLSASVGDRVTITCRASQKIGKRLRWYQQKPGKAPKLLIYGASRLQSGVPSRFSGSGSGTDFTLTISSLQPEDFATYYCQQNLERPNTFGQGTKVEIKR

SEQ ID NO: 329 - protein, 108 aa, Homo sapiens
DIQMTQSPSSLSASVGDRVTITCRASQPIGSRILWYQQKPGRAPKLLIYHASKLQSGVPSRFSGSGSGTDFTLTISSLQPEDFATYYCQQRKYQPHTFGQGTKVEIKR

SEQ ID NO: 330 - protein, 108 aa, Homo sapiens; identical to SEQ ID NO: 326

SEQ ID NO: 331 - protein, 108 aa, Homo sapiens
DIQMTQSPSSLSASVGDRVTITCRASQMIGKRLRWYQQKPGKAPKLLIYFASRLQSGVPSRFSGSGSGTDFTLTISSLQPEDFATYYCQQSRQHPHTFGQGTKVEIKR

SEQ ID NO: 332 - protein, 108 aa, Homo sapiens
DVQMTQSPSSLSASVGDRVTITCRASQLIRKRLRWYQQKPGKAPKLLIYHSSKLQSGVPSRFSGSGSGTDFTLTISSLQPEDFATYYCQQGHSRPFTFGQGTKVEIKR

SEQ ID NO: 333 - protein, 108 aa, Homo sapiens
DIQMTQSPSSLSASVGDRVTITCRASQRIHNRLSWYQQKPGKAPKLLIYAASKLQSGVPSRFSGSGSGTDFTLTISSLQPEDFATYYCQQTSYRPHTFGQGTKVEIKR

SEQ ID NO: 334 - protein, 108 aa, Homo sapiens
DIQMTQSPSSLSASVGDRVTITCRASQSIFKRLRWYQQKPGKAPKLLIYASSVLQSGVPSRFSGSGSGTDFTLTISSLQPEDFATYYCQQNVAIPFTFGQGTKVEIKR

SEQ ID NO: 335 - protein, 108 aa, Homo sapiens
DIQMTQSPSSLSASVGDRVTITCRASQNINERLLWYQQKPGKAPTLLIYHSSRLQSGVPSRFSGSGSGTDFTLTISSLQPEDFATYYCQQKYKRPYTFGQGTKVEIKR

SEQ ID NO: 336 - protein, 108 aa, Homo sapiens
DIQMTQSPSSLSASVGDRVTITCRASQNIGRKLRWYQQKPGKAPKLLIYGTSRLQSGVPSRFSGSGSGTDFTLTISSLQPEDFATYYCQQNLHLPSTFGQGTKVEIKR

SEQ ID NO: 337 - protein, 108 aa, Homo sapiens
DIQMTQSPSSLSASVGDRVTITCRASQDIERRLLWYQQKPGKAPKLLIYSTSRLQSGVPSRFSGSGSGTDFTLTISSLQPEDFATYYCQQRHTSPHTFGQGTKVEIKR

SEQ ID NO: 338 - protein, 108 aa, Homo sapiens
DIQMTQSPSSLSASVGDRVTITCRASQNITNRLRWYQQKPGKAPKLLIYRSSVLQSGVPSRFSGGGSGTDFTLTISSLQPEDFATYYCQQHNYQPHTFGQGTKVEIKR

SEQ ID NO: 339 - protein, 108 aa, Homo sapiens
DIQMTQSPSSLSASVGDRVTITCRASQSIGRGLAWYQQKPGKAPKLLIYMGSRLQSGVPSRFSGSGSGTDFTLTISSLQPEDFATYYCQQQRHLPRTFGQGTKVEIKR

SEQ ID NO: 340 - protein, 108 aa, Homo sapiens
DIQMTQSPSSLSASVGDRVTITCRASQPIGHRLRWYQQKPGKAPKLLIYRASKLQSGVPSRFSGSGSGTDFTLTISSLQPEDFATYYCQQLYKQPLTFGQGTKVEIKR

SEQ ID NO: 341 - protein, 108 aa, Homo sapiens
DIQMTQSPSSLSASVGDRVTITCRASQPISRRLLWYQQKPGKAPKLLIYGASRLQSGVPSRFSGSGSGTDFTLTISSLQPEDLATYYCQQRETNPHTFGQGTKVEIKR

SEQ ID NO: 342 - protein, 108 aa, Homo sapiens
DIQMAQSPSSLSASVGDRVTITCRASQVIGKELAWYQQKPGKAPKLLIYHVSRLRSGVPSRFSGSGSGTDFTLTISSLQPEDSATYYCQQKVAYPFTFGQGTKVEIKR

SEQ ID NO: 343 - protein, 108 aa, Homo sapiens
DIQMTQSPSSLSASVGDRVTITCRASQDIVDRLSWYQQKPGKAPKLLIYRSSRLRSGVPSRFSGSGSGTDFTLTISSLQPEDFATYYCQQRLRFPITFGQGTKVEIKR

SEQ ID NO: 344 - protein, 108 aa, Homo sapiens
DIQMTQSPSSLSASVGDRVTITCRASRPISTSLVWYQQKPGKAPKLLIYNASNLQSGVPSRFSGSGSGTDFTLTISSLQPEDFATYYCQQSQTLPVTFGQGTKVEIKR

SEQ ID NO: 345 - protein, 108 aa, Homo sapiens
DIQMTQSPSSLSASVGDRVTITCRASQAIWRSLNWYQQKPGKAPKLLIYRSSRLQSGVPSRFSGSGSGTDFTLTISSLQPEDFATYYCQQYSNRPYTFGQGTKVEIKR

SEQ ID NO: 346 - protein, 108 aa, Homo sapiens
DIQMTQSPSSLSASVGDRVTITCRASQDIGDRLRWYQQKPGKAPKLLIYHGSRLESGVPSRFSGSRSGTDFTLTISSLQPEDFATYYCQQQWFRPYTFGQGTKVEIKR

SEQ ID NO: 347 - protein, 108 aa, Homo sapiens
DIQMTQSPSSLSASVGDRVTITCRASQKIGQHLHWYQQKPGKAPKLLIYRTSILQSGVPSRFSGSGSGTDFTLTISSLQPEDFATYYCQQNHRRPYTFGQGTKVEIKR

SEQ ID NO: 348 - protein, 108 aa, Homo sapiens
DIQMTQSPSSLSASVGDRVTITCRASQDIGDRLRWYQQNPGKAPKLLIYHGSRLESGVPSRFSGNRSGTDFTLTISSLQPEDFATYYCQQQWFRPYTFGQGTKVEIKR

SEQ ID NO: 349 - protein, 108 aa, Homo sapiens
DIQMTQSPSSLSASVGDRVTITCRASQDIGGRLRWYQQKPGKAPKLLIYHGSRLDSGVPSRFSGSRSGADFTLTISSLQPEDFATYYCQQQWYRPYTFGQGTKVEIKR

SEQ ID NO: 350 - protein, 108 aa, Homo sapiens
DIQMTQSPSSLSASVGDRVTITCRASQDIGDRLRWYQQKPGKAPKLLIYHGSRLESGVPSRFSGSRSGTDFTLTISSLQPEDFATYYCQQQWLRPYTFGQGTRVEIKR

SEQ ID NO: 351 - protein, 108 aa, Homo sapiens
DIQMTQSPSSLSASVGDRVTITCGASQDIGDRLRWYQQKPGKAPKLLIYHGSRLESGVPSRFSGSRSGTDFTLTISSLQPEDFATYYCQQQWFRPYTFGQGTKVEIKR

SEQ ID NO: 352 - protein, 108 aa, Homo sapiens
DIQMTQSPSSLSASVGDRVTITCRASQDIGDRLRWYQQKPGKAPKLLIYHGSRLDSGVPSRFSGSRSGTDYTLTISSLQPEDFATYYCQQQWFRPYTFGQGTKVEIKR

SEQ ID NO: 353 - protein, 108 aa, Homo sapiens
DIQMTQSPSSLSASVGDRVTITCRASQDIGDRLRWYQQKPGRAPKLLIYHGSRLESGVPSRFSGSRSVTDFTLTISSLQPEDFATYYCQQQWFRPYTFGQGTKVEIKR

SEQ ID NO: 354 - protein, 108 aa, Homo sapiens
DIQMTQSPSSLSASVGDRVTITCRASQVIGDRLRWYQQKPGKAPKLLIYHGSRLESGVPSRFSGSRSGTDFTLTISSLQPEDFATYYCQQQWFRPYTFGQGTKVEIKR

SEQ ID NO: 355 - protein, 108 aa, Homo sapiens
DIQMTQSPSSLSASVGDRVTITCRASQDIGDRLRWYQQKPGKAPKLLIYHGSRLESGVPSRFSGNRSGTVFTLTISSLQPEDFATYYCQQQWFRPYTFGQGTKVEIKR

SEQ ID NO: 356 - protein, 108 aa, Homo sapiens
DIQMTQSPSSLSASVGDRVTITCRASQDIGDRLRWYQQKPGKAPKLLIYHGSRLESGVPSRFRGSRSGTDFTLTISSLQPEDFATYYCQQQWFRPYTFGQGTKVEIKR

SEQ ID NO: 357 - protein, 108 aa, Homo sapiens
DIQMTQSPSSLSASVGDRVTITCRASQDIGDRLRWYQQKPGKAPKLLIYHGSRLESGVPSRFSGSRYGTNFTLTISSLQPEDFATYYCQQQWFRPYTFGQGTKVEIKR

SEQ ID NO: 358 - protein, 108 aa, Homo sapiens
DIQMTQSPSSLSASVGDRVTITCRASQDIGDRLRWYQQKPGKAPKLLIYHGSRLESGVPSRFSGSRFGTDFTLTISSLQPDDFATYYCQQQRFRPYTFGQGTKVEIKR

SEQ ID NO: 359 - protein, 108 aa, Homo sapiens
DIQMTQSPSSLSASVGDRVTITCRASQDIGDRLRWYQQKPGKAPKLMIYHGSRLESGVPSRFSGSRSGTDFTLTISSLQPEDFATYYCQQQWFRPYTFGQGTKVEIKR

SEQ ID NO: 360 - protein, 108 aa, Homo sapiens
DIQMTQSPSSLSASVGDRVTITCRASQDIGDRLRWYQQKPGKAPKLLIYHGSRLESGVPSRFSGGRSGTDFTLTISSLQLEDFATYYCQQQWFRPYTFGQGTKVEIKR

SEQ ID NO: 361 - protein, 108 aa, Homo sapiens
DIQMTQSPSSLSASVGDRVTITCRASRDIGDRLRWYQQKPGKAPKLLIYHGSRLESGVPSRFSGSRSGTDFTLTISSLQPEDFATYYCQQQWFRPYTFGQGTKVEIKR

SEQ ID NO: 362 - protein, 108 aa, Homo sapiens
DIQMTQSPSSLSASVGDRVTITCRASQDIGYRLRWYQQKPGKAPMLLIYHGSRLESGVPSRFSGRRSGTDFTLTISSLQPEDFATYYCQQQWFRPYTFGQGTKVEIKR

SEQ ID NO: 363 - protein, 108 aa, Homo sapiens
DIQMTQSPSSLSASVGDRVTITCRASQDIGDRLRWYQQKPGKAPKLLIYHGSRLESGVPSRFSGSRSGTDFTLTISSLLPEDFATYYCQQQRFRPYTFGQGTKVEIKR

SEQ ID NO: 364 - protein, 108 aa, Homo sapiens
DIQMTQSPSSLSASVGDRVSITCRASQDIGDRLRWYQQKPGKAPKLLIYHGSRLESGVPSRFRGSRSGTDFNLTISSLQPGDFATYYCQQQWFRPYTFGQGTKVEIKR

SEQ ID NO: 365 - protein, 108 aa, Homo sapiens
DIQMTQSPSSLSASVGDRVTITCRASQDIGDRLRWYQQKPGKAPKLLIYHGSRLESRVPSRFSGNRSGTDFTLSISSLQPEDFATYYCQQQRFRPYTFGQGTKVEIKR

SEQ ID NO: 366 - protein, 108 aa, Homo sapiens
DIQMTQSPSSLSASVGDRVTITCRASQDIGDRLRWYQQKPGKAPKLMIYHGSRLESGVPSRFSGSRSGTDFTLTISSLQPEDFATYYCQQQWSRPYTFGQGTKVEIKR

SEQ ID NO: 367 - protein, 108 aa, Homo sapiens
DIQMTQSPSSLSASVGDRVAITCRASQDIGDRLRWYHQKPGKAPKLLIYHGSRLESGVPSRFSGSRSGTDFTLTISSLQPEDFATYYCQQQWFRPYTFGQGTKVEIKR

SEQ ID NO: 368 - protein, 108 aa, Homo sapiens
DIQMTQSPSSLSASVGDRITITCRASQDIGDRLRWYQQRPGKAPKLLVYHGSRLESGVPSRFSGSRSGTDFTLTISSLQPEDYATYYCQQQWFRPYTFGQGTKVEIKR

SEQ ID NO: 369 - protein, 108 aa, Homo sapiens
DIQMTQSPSSLSASVGDRVTITCRASQDIGDRLRWYQQKPGKAPKLLIYHGSRLESGVPSRFSGGRSGTDFTLTISSLQPEDSATYYCQQQWLRPYTFGQGTKVEIKR

SEQ ID NO: 370 - protein, 108 aa, Homo sapiens; identical to SEQ ID NO: 359

SEQ ID NO: 371 - protein, 108 aa, Homo sapiens
DIQMTQSPSSLSASVGDRVTITCRASQDIGDRLRWYQQKPGKAPKLMIYHGSRLESGVPPRFSGSRSGTDFTLNISSLQPDDFATYYCQQQWFRPYTFGQGTKVEIKR

SEQ ID NO: 372 - protein, 108 aa, Homo sapiens
DIQMTQSPSSLSASVGDRVTITCRASQDIGDRLRWYQQKPGKAPRLLIYHGSRLESGVPSRFSGSRSGTDFTLTFSSLQPEDFATYYCQQQWIRPYTFGQGTKVEIKR

SEQ ID NO: 373 - protein, 108 aa, Homo sapiens; identical to SEQ ID NO: 361

SEQ ID NO: 374 - protein, 108 aa, Homo sapiens
DIQMTQSPTSLSASVGDRVTITCRASQDIGDRLRWYQQRPGKAPKLLIYHGTRLESGVPSRFSGSRSGTDFTLTISSLQPEDFATYYCQQQWFRPYTFGQGTKVEIKR

SEQ ID NO: 375 - protein, 108 aa, Homo sapiens
DIQMTQSPSSLSASVGDRVTITCRASQDIGDRLRWYQQKPGKAPKLLIYHGSRLESGIPSRFSGSRSGTDFTLTISSLQPEDFATYYCQQQWFRPYTFGQGTKVEIKR

SEQ ID NO: 376 - protein, 108 aa, Homo sapiens
DIQMTQSPSSLSASVGDRVTITCRASQDIDDRLRWYQQKPGKAPKLLIYHGSRLDSGVPSRFSGSRSGTDFTLTISSLQPEDFATYYCQQQWFRPYTFGQGTKVEIKR

SEQ ID NO: 377 - protein, 108 aa, Homo sapiens
DIQMTQSPSSLSASVGDRVTITCRASQDIGDRLRWYQQKPGKAPKLLIYHGSRLDSGVPSRLSGSRSGTDFTLTISSLQPEDFATYYCQQQWFRPYTFGQGTKVEIKR

SEQ ID NO: 378 - protein, 108 aa, Homo sapiens
DIQMTQSPSSLSASVGDRVTITCRASQDIGDRLRWYQQKPGKAPKLLIYHGSRLESGVPSRFSGGRSGTDFTLTIRSLQPEDFATYYCQQQWFRPYTFGQGTKVEIKR

SEQ ID NO: 379 - protein, 108 aa, Homo sapiens
DIQMTQSPSSLSASVGDRVTITCRASRDIGDRLRWYQQKPGKAPKLLIYHGSRLESGVPSRFSGSRSGTDFTLTISRLQPEDFATYYCQQQWFRPYTFGQGTKVEIKR

SEQ ID NO: 380 - protein, 108 aa, Homo sapiens
DIQMTQSPSSLSASVGDRVTITCRASQDIGDRLRWYQQKPGKAPKLLIYHGSRLESGVPSRFSGGRSGTDFTLTISSLQPEDFATYYCQQQRFRPYTFGQGTKVEIKR

SEQ ID NO: 381 - protein, 108 aa, Homo sapiens
DIQMTQSPSSLSATVGDRVTITCRASQDIGDRLRWYQQKTGKAPKLLIYHGSRLESGVPSRFSGSRSGTDFTLTISSLQPEDFATYYCQQQWFRPYTFGQGTKVEIKR

SEQ ID NO: 382 - protein, 108 aa, Homo sapiens
DIQMTQSPSRLSATVGDRVTITCRASQDISDRLRWYQQKPGKAPKLLIYHGSRLESGVPSRFSGSRSGTDFALTISSLQPEDFATYYCQQQWFRPYTFGQGTKVEIKR

SEQ ID NO: 383 - protein, 108 aa, Homo sapiens
DIQMTQSPSSLSASVGDRVTITCRASQDIGDRLRWYQQKPGKAPKLLIYHGSRLESGVPSRFSGSRSGTDFALTISSLQPEDFATYYCQQQWFRPYTFGPGTKVEIKR

SEQ ID NO: 384 - protein, 108 aa, Homo sapiens; identical to SEQ ID NO: 361

SEQ ID NO: 385 - protein, 108 aa, Homo sapiens
DIQMTQSPSSLSASVGDRVTITCRASQNIGGRLVWYQQKPGKAPKLLIYTPSPLQSGVPSRFSGSGSGTDFTLTISSLQPEDFATYYCQQRHTSPFTFGQGTKVEIKR

SEQ ID NO: 386 - protein, 108 aa, Homo sapiens
DIQMTQSPSSLSASVGDRVTITCRASQNIGGRLVWYQQKPGKAPKLLIYTPSPLQSGVPSRFSGSGSGTDFTLTISSLQPEDSATYYCQQRYSLPFTFGQGTKVEIKR

SEQ ID NO: 387 - protein, 108 aa, Homo sapiens
DIQMTQSPSSLSASVGDRVTITCRASQPIGSMLVWYQQKPGKAPKLLIYTPSSLQSGVPSRFSGSGSGTDFTLTISSLQPEDFATYYCQQKYMEPHTFGQGTKVEIKQ

SEQ ID NO: 388 - protein, 108 aa, Homo sapiens
DIQMTQSPSSLSASVGDRVTITCRASQQIGQLLNWYQQKPGKAPKLLIYAGSRLQSGVPSRFSGSGSGTDFTLTISSLQPEDFATYYCQQMRQTPVTFGQGTKVEIKR

SEQ ID NO: 389 - protein, 108 aa, Homo sapiens
DIQMSQSPSSLSASVGDRVTITCRASQDIGNMLAWYQQKPGKAPKPLIYYASYLQSGVPSRFSGSGSGTDFTLTISSLQPEDFATYYCQQLGAKPHTFGQGTKVEIKR

SEQ ID NO: 390 - protein, 108 aa, Homo sapiens
DIQMTQSPSSLSASVGDRVTITCRASQVIGNALRWYQQKPGKAPKLLIYYGSYLQSGVPSRFSGSGSGTDFTLTISSLQPEDFATYYCQQIHFKPFTFGQGTKVEIKR

SEQ ID NO: 391 - protein, 108 aa, Homo sapiens
DIQMTQSPSSLSASVGDRVTITCRASQYIGGSLRWYQQKPGKAPKLLIYSGSTLQSGVPSRFSGSGSGTDFTLTISSLQPEDFATYYCQQTWVSPMTFGQGTKVEIKR

SEQ ID NO: 392 - protein, 108 aa, Homo sapiens
  VARIANT 28, 30, 32, 34, 36, 93: Xaa = Any Amino Acid
DIQMTQSPSSLSASVGDRVTITCRASQXIXTXLXWXQQKPGKAPTLLIYNSSQLQSGVPSRFSGSGSGTDFTLTISSLQPEDFATYYCQQTWXRPMTFGQGTKVEIKR

SEQ ID NO: 393 - protein, 108 aa, Homo sapiens
DIQMTQSPSSLSASVGDRVTITCRASQNIGGRLVWYQQKPGKAPKLLIYTPSPLQSGVPSRFSGSGSGTDFTLTISSLLPEDFATYYCQQRHSAPYTFGQGTKVEIKR

SEQ ID NO: 394 - protein, 108 aa, Homo sapiens
DIQMTQSPSSLSASVGDRVTITCRASQNIGGRLVWYQQKPGKAPKLLIYTPSPLQSGVPSRFSGSGSGTDFTLTISSLQPEDFATYYCQQRQQQPYTFGQGTKVEIKR

SEQ ID NO: 395 - protein, 108 aa, Homo sapiens
DIQMTQSPSSLSASVGDRVTITCRASQNIGGRLVWYQQKPGKAPKLLIYTPSPLQSGVPSRFSGSGSGTDFTLTISSLQPEDFATYYCQQRASRPYTFGQGTKVEIKR

SEQ ID NO: 396 - protein, 106 aa, Homo sapiens
DIQMTQSPSSLSASVGDRVTITCRASQNIGGRLVWYQQKPGKAPKLLIYTPSPLQVPSRFSGSGSGTDFTLTISSLQPEDFATYYCQQRYVQPYTFGQGTKVEIKR

SEQ ID NO: 397 - protein, 108 aa, Homo sapiens
DIQMTQSPSSLSASVGDRVTITCRASQNIGGRLVWYQQKPGKAPKLLIYTPSPLQSGVPSRFSGSGSGTDFTLTISSLQPEDFATYYCQQRYKPPYTFGQGTKVKIKR

SEQ ID NO: 398 - protein, 108 aa, Homo sapiens
DIQMTQSPSSLSASVGDRVTITCRASQNIGGRLVWYQQKPGKAPKLLIYTPSPLQSGVPSRFSGSGSGTDFTLTISSLQPEDFATYYCQQRVRAPYTFGQGTKVEIKR

SEQ ID NO: 399 - protein, 108 aa, Homo sapiens
DIQMTQSPSSLSASVGDRVTITCRASQNIGSKLVWYQQKPGKASKLLIYTPSRLQSGVPSRFSGSGSGTDFTLTISSLQPEDFATYYCQQRFMTPYTFGQGTKVEIKR

SEQ ID NO: 400 - protein, 108 aa, Homo sapiens
DIQMTQTPSSLSASVGDRVTITCRASQNIGKQLLWYQQKPGKAPRLLIYCPPPLQSGVPSRFSCSGSGTDFTLTISSLQPEDFATYYCQQHASRPFTFGQGTKVEIKR

SEQ ID NO: 401 - protein, 108 aa, Homo sapiens
DIQMTQSPSSLSASVGDRVTISCRASQNIGTQLHWYQQKPGKAPRLLIYGSSFLQSGVPSRFSGSGSGTDFTLTISSLQPEDFATYYCQQVMLGPTTFGQGTKVEIKR

SEQ ID NO: 402 - protein, 108 aa, Homo sapiens
DIQMTQSPSSLSASVGDRVTITCRASQNIHGMLRWYQQKPGKAPKLLIYTPSPSQSGVPSRFSGSGSGTDFTLTISSLQPEDFATYYCQQTATWPFTFGQGTKVEIKR

SEQ ID NO: 403 - protein, 108 aa, Homo sapiens
DIQMTQSPSSLSASVGDRVTITCRASQPIGNKLRWYQQKPGKAPKLLIYSPSPLQSGVPSRFSGSGSGTDYTLTISSLQPEDFATYYCQQTWSFPGTFGQGTKVEIKR

SEQ ID NO: 404 - protein, 108 aa, Homo sapiens
  VARIANT 92: Xaa = Any Amino Acid
DIQMTQSPSSLSASVGDRVTITCQASQPIDGRLVWYQQKPGKASKLLIYVPSGLQSGVPSRFSGSGSGTDFTLTISSLQPEDFATYYCQQRXTPPYTFGQGTKVEIKR

SEQ ID NO: 405 - protein, 108 aa, Homo sapiens
DIQMTQSPSSLSASVGDRVTITCRASQSIGGRLVWYQQKPGKAPKLLIYTPPPLQSGVPSRFSGSGSGTDFTLTISSLQPEDFATYYCQQRYLRPFTFGQGTKVEIKR

SEQ ID NO: 406 - protein, 108 aa, Homo sapiens
DIQMTQSPSSLSASVGDRVTITCRASQSIGPWLSWYQQKPGKAPKLLIYQVSRLQSGVPSRFSGSGSGTDFTLTISSLQPEDFATYYCQQNLAPPYTFGQGTKVEIKR

SEQ ID NO: 407 - protein, 108 aa, Homo sapiens
DIQMTQSPSSLSASVGDRVTITCRASRNIGGRLVWYQQKPGKAPKLLIYTPSPLQSGVPSRFSGSGSGTDFTLTISSLQPEDFATYYCQQRHNEPFTFGQGTKVEIKR

SEQ ID NO: 408 - protein, 108 aa, Homo sapiens
  VARIANT 30, 34, 36, 51, 61, 91, 96: Xaa = Any Amino Acid
DIQMTQSPSSLSASVGDRVTITCRASQNIXTLLXWXQQKPGKAPKLLIYAXSRLQSGVPSXFSGSGSGTDFTLTISSLQPEDFATYYCQQXYRHPXTLGQGTKVEIKR

SEQ ID NO: 409 - protein, 108 aa, Homo sapiens
DIQMTQSPSSLSASVGDRVTITCRASQDIKSHLRWYQQKPGKAPKLLIYTPSSLQSGVPSRFSGSGSGTDFTLTISSLQPEDSATYYCQQVLTVPYTFGQGTKVEIKR

SEQ ID NO: 410 - protein, 108 aa, Homo sapiens
DIQMTQSPSSLSASVGDRVTITCRASQDIGRWLSWYQQKPGKAPKLLIYAGSQLQSGVPSRFSGSGSGTDFTLTISSLQPEDFATYYCQRSWDPPTTFGQGTKVEIKR

SEQ ID NO: 411 - protein, 108 aa, Homo sapiens
DIQMTQSPSSLSASVGDRVTITCRASQDINRQLVWYQQKPGKAPKLLIYTPSSLQSGVPSRFSGSGSGTDFTLTISSLQPEDYATYYCQQKYRYPFTFGQGTKVEIKR

SEQ ID NO: 412 - protein, 108 aa, Homo sapiens
DIQMTQSPSSLSASVGDRVTITCRASQDISRFLNWYQQKPGKAPKLLIYWTSLLQSGVPSRFSGSGSGTDFTLTISSLQPEDFATYYCQQSRHHPTTFGQGTKVEIKR

SEQ ID NO: 413 - protein, 108 aa, Homo sapiens; identical to SEQ ID NO: 389

SEQ ID NO: 414 - protein, 108 aa, Homo sapiens
DIQMTQSPSSLSASVGDRVTITCRASQEINNMLVWYQQKPGKAPKLLIYAPSGLQSGVPSRFSGSGSGTDFTLTISSLQPEDFATYYCQQRRYPPFTFGQGTKVEIKR

SEQ ID NO: 415 - protein, 108 aa, Homo sapiens
DIQMTQSPSSLSASVGDRVTITCRASQEIGSHLRWYQQKPGKAPKLLIYQESQLQSGVPSRFSGSGSGTDFTLTISSLQPEDFATYYCQQTWNSPMTFGQGTKVEIKR

SEQ ID NO: 416 - protein, 108 aa, Homo sapiens
DIQMTQSPSSLSASVGDRVTITCRASQGIGRHLRWYQQKPGKAPKLLIYSPSGLQGGVPSRFSGSGSGTDYTLTISSLQPEDYATYYCQQVYSPPFTFGQGTKVEIKR

SEQ ID NO: 417 - protein, 108 aa, Homo sapiens
DIQMTQSPSSLSASVGDRVTITCRASQKIGNMLAWYQQKPGKAPKLLIYKYSKLQSGVPSRFSGSGSGTDFTLTISSLQPEDFATYYCQQLAVPPHTFGQGTKVEIKR

SEQ ID NO: 418 - protein, 108 aa, Homo sapiens
DIQMTQSPSSLSASVGDRVTITCRASQYIQMRLRWYQQKPGKAPRLLIYGASMLQSGVPSRFSGSGSGTDFTLTISSLQPEDFATYYCQQDWTAPHTFGQGTKVEIKR

SEQ ID NO: 419 - protein, 108 aa, Homo sapiens
DIQMTQSPSSLSASVGDRVTITCRASQDIGQLLNWYQQKPGKAPKLLIYASSRLQSGVPSRFSGSGSGTDFTLTISSLQPEDFATYYCQQRTYNPSTFGPGTKVEIKR

SEQ ID NO: 420 - protein, 108 aa, Homo sapiens
DIQMTQSPSSLSASVGDRVTITCRASQDIGALLRWYQQKPGKAPKLLIYTPSELQSGVPSRFSGSGSGTDFTLTISSLQPEDFATYYCQQVFRSPYTFGQGTKVEIKR

SEQ ID NO: 421 - protein, 108 aa, Homo sapiens
DIQMTQSPSSLSASVGDRVTITCRASQDIGAQLRWYRQKPGKAPKLLIYAPSALQSGVPSRFSGSGSGTDFTLTISSLQPEDFATYYCQQVALRPYTFGQGTRVEIKR

SEQ ID NO: 422 - protein, 108 aa, Homo sapiens
DIQMTQSPSSLSASVGDRVTITCRASQDIGHKLRWYQQKPGKAPKLLIYTPSTLQSGVPSRFSGSGSGTDFTLTISSLQPEDFATYYCQQTWTPPYTFGQGTKVEIKR

SEQ ID NO: 423 - protein, 108 aa, Homo sapiens
DIQMTQSPSSLSASVGDRVTITCRASQDIDTHLRWYQQKPGKAPKLLIYGSSFLQSGVPSRFSGSGSGTDFTLTISSLQPEDFATYYCQQTWARPMTFGQGTKVEIKR

SEQ ID NO: 424 - protein, 108 aa, Homo sapiens
DIQMTQSPSSLSASVGDRVTITCRASQDIKGMLVWYQQKPGKAPKLLIYTPSRLQSGVPSRFSGSGSGTDFTLTISSLQPEDFATYYCQQTWVSPQTFGQGTKVEIKR

SEQ ID NO: 425 - protein, 108 aa, Homo sapiens
DIQMTQSPSSLSASVGDRVTITCRASQDIKSHLRWYQQKPGKAPKLLIYTPSSLQSGVPSRFSGSGSGTDFTLTISSLQPEDFATYYCQQVSSTPYTFGQGTKVEIKR

SEQ ID NO: 426 - protein, 108 aa, Homo sapiens; identical to SEQ ID NO: 415

SEQ ID NO: 427 - protein, 108 aa, Homo sapiens
DIQMTQSPSSLSASVGDRVTITCRASQEIGGNLVWYQQKPGKAPKLLIYAPSRLQSGVPSRFSSSGSGTDFTLTISSLQPEDFATYYCQQKFSYPFTFGQGTKVEIKR

SEQ ID NO: 428 - protein, 108 aa, Homo sapiens; identical to SEQ ID NO: 414

SEQ ID NO: 429 - protein, 108 aa, Homo sapiens
DIQMTQSPSSLSASVGDRVTITCRASQWIGNHLRWYQQKPGKAPTLLIYGSSRLQSGVPSRFSGSGSGTDFTLTISSLQPEDFATYYCQQTWNSPMTFGQGTKVEIKR

SEQ ID NO: 430 - protein, 108 aa, Homo sapiens
DIQMTQSPSSLSASVGDRVTITCRASQAIDIHLRWYQQKPGKAPKLLIYHASSLQSGVPSRFSGSGSGTDFTLTISSLQPEDFATYYCQQTYRSPMTFGQGTKVEIKR

SEQ ID NO: 431 - protein, 108 aa, Homo sapiens
  VARIANT 50: Xaa = Any Amino Acid
DIQMTQSPSSLSASVGDRVTITCRASQAIGQSLRWYQQKPGKAPTLLIYXSSNLQSGVPSRFSGSGSGTDFTLTISSLQPEDFATYYCQQTWVSPMTFGQGTKVEIKR

SEQ ID NO: 432 - protein, 108 aa, Homo sapiens; identical to SEQ ID NO: 391

SEQ ID NO: 433 - protein, 108 aa, Homo sapiens
  VARIANT 38: Xaa = Any Amino Acid
DIQMTQSPSSLSASVGDRVTITCRASQYINAHLRWYQXKPGKAPKLLIYMSSYLQSGVPSRFSGSGSGTDFTLTISSLQPEDFATYYCQQTWSSPMTFGQGTKVEIKR

SEQ ID NO: 434 - protein, 108 aa, Homo sapiens
DIQMTQSPSSLSASVGDRVTITCRASQGIMYHLRWYQQKPGKAPRLLIYHGSTLQSGVPARFSGSGSGTDFTLTISSLQPEDFATYYCQQTWNAPLTFGQGTKVEIKR

SEQ ID NO: 435 - protein, 108 aa, Homo sapiens
DIQMTQSPSSLSASVGDRVTITCRASQGIGNSLRWYQQKPGKAPKLLIYYSSHLQSGVPSRFSGSGSGTDFTLTISSLQPEDFATYYCQQIRTKPFTFGQGTKVEIKR

SEQ ID NO: 436 - protein, 108 aa, Homo sapiens
DIQMTQSPSSLSASVGDRVTITCRASQKIMTHLRWYQQKPGKAPKLLIYGGSHLQSGVPSRFSGSGSGTDFTLTISSLQPEDFATYYCQQTWVSPMTFGQGTKVEIRR

SEQ ID NO: 437 - protein, 108 aa, Homo sapiens
DIQMTQSPSSLSASVGDRVTITCRASQRIGHHLRWYQQKPGKAPKLLIYSASALQSGVPSRFSGSGSGTDFTLTISSLQPEDFATYYCQQTWNAPMTFGQGTKVEIKR

SEQ ID NO: 438 - protein, 108 aa, Homo sapiens
DIQMTQSPSSLSASVGDRVTITCRASQRIGLMLNWYQQKPGKAPRLLIYAASRLQSGVPSRFSGSGSGTDFTLTISSLQPEDFATYYCQQMLHPPVTFGQGTKVEIKR

SEQ ID NO: 439 - protein, 108 aa, Homo sapiens
  VARIANT 3, 38: Xaa = Any Amino Acid
DIXMTQSPSSLSASVGDRVTITCRASQRIEGKLLWYQXKPGKAPKLLIYCPSNLQSGVPSRFSCSGSGTDFTLTISSLQPEDFATYYCQQKFREPSTFGQGTKVEIKR

SEQ ID NO: 440 - protein, 108 aa, Homo sapiens; identical to SEQ ID NO: 405

SEQ ID NO: 441 - protein, 108 aa, Homo sapiens
DIQMTQSPSSLSASVGDRVTITCRASQSIGTLLNWYQQKPGKAPKLLIYASSRLQSGVPSRFSGSGSGTDFTLTISSLQPEDFATYYCQQMNRVPITFGQGTKVEIKR

SEQ ID NO: 442 - protein, 108 aa, Homo sapiens
DIQMTQSPSSLSASVGDRVTITCRASQSIGMLLSWYQQKPGKAPKLLIYAVSRLQSGVPSRFSGSGSGTDFTLTISSLQPEDYATYYCQQMQRPPITFGQGTKVEIKR

SEQ ID NO: 443 - protein, 108 aa, Homo sapiens
DIQMTQSPSSLSASVGDRVTITCRASQSIGVNLLWYQQIPGKAPRLLIYGASYLQSGVPSRFSGSGSGTDFTLTISSLQPEDFATYYCQQYFFAPLTFGQGTKVEIKR

SEQ ID NO: 444 - protein, 108 aa, Homo sapiens
DIQMTQSPSSLSASVGDRVTITCRASQSIGHNLVWYQQKPGKAPKLLIYTPSPLQSGVPSRFSGSGSGTDFTLTISSLQPEDFATYYCQQKYTPPYTFGQGTKVEIKR

SEQ ID NO: 445 - protein, 108 aa, Homo sapiens
  VARIANT 17: Xaa = Any Amino Acid
DIQMTQSPSSLSASVGXRVTITCRASQSIGVQLRWYQQKPGKAPKLLIYHGSQLQSGVPSRFSGSGSGTDFTLTISSLQPEDFATYYCQQNWARPITFGQGTKVEIKR

SEQ ID NO: 446 - protein, 108 aa, Homo sapiens
DIQMTQSPSSLSASVGDRVTITCRASQSIATSLRWYQQKPGKAPKLLIYHSSVLQSGVPSRFSGSGSGTDFTLTISSLQPEDFATYYCQQTWVVPMTFGQGTKVEIKR

SEQ ID NO: 447 - protein, 108 aa, Homo sapiens
DIQMTQSPSSLSASVGDRVTITCRASQSIKGHLVWYQQKPGKAPMLLIYSPSSLRSGVPSRFSGSGSGTDFTLTISSLQPEDFATYYCQQVYEKPFTFGQGTKVEIKR

SEQ ID NO: 448 - protein, 108 aa, Homo sapiens
  VARIANT 93: Xaa = Any Amino Acid
DIQMTQSPSSLSASVGDRVTITCRASQPIHGALRWYQQKPGKAPMLLIYTPSQLQSGVPSRFSGSGSGTDFTLTISSLQPEDFATYYCQQVGXKPYTFGQGTKVEIKR

SEQ ID NO: 449 - protein, 108 aa, Homo sapiens
DIQMTQSPSSLSASVGDRVTITCQASQPIDGRLVWYQQKPGKASKLLIYVPSGLQSGVPSRFSGSGSGTDFTLTISSLQPEDFATYYCQQRHTPPYTFGQGTKVEIKR

SEQ ID NO: 450 - protein, 108 aa, Homo sapiens
DIQMTQSPSSLSASVGDRVTITCRASQPINNWLNWYQQKPGKAPKLLIYATSRLQSGVPSRFSGSGSGTDFTLTISSLQPEDFATYYCQPSWTPPPTFGQGTKVEIKR

SEQ ID NO: 451 - protein, 108 aa, Homo sapiens
DIQMTQSPSSLSASVGDRVTITCRASQPIKMMLSWYQQKPGKAPKLLIYNNSTLQSGVPSRFSGSGSGTDFTLTISSLQPEDFATYYCQQYRRWPYTFSQGTKVEIKR

SEQ ID NO: 452 - protein, 108 aa, Homo sapiens; identical to SEQ ID NO: 387

SEQ ID NO: 453 - protein, 108 aa, Homo sapiens; identical to SEQ ID NO: 388

SEQ ID NO: 454 - protein, 108 aa, Homo sapiens
  VARIANT 82: Xaa = Any Amino Acid
DIQMTQSPSSLSASVGDRATITCRASQQIGAHLRWYQQKPGKAPKLLIYQSSQLQSGVPSRFSGSGSGTDFTLTISSLQPEXFATYYCQQTWASPMTFGQGTKVEIKR

SEQ ID NO: 455 - protein, 108 aa, Homo sapiens; identical to SEQ ID NO: 398

SEQ ID NO: 456 - protein, 108 aa, Homo sapiens
  VARIANT 27: Xaa = Any Amino Acid
DIQMTQSPSSLSASVGDRVTITCRASXNIGGRLVWYQQKPGKAPKLLIYTPSPLQSGVPSRFSGSGSGTDFTLTISSLQPEDFATYYCQQRSVSPYTFGQGTKVEIKR

SEQ ID NO: 457 - protein, 108 aa, Homo sapiens
DIQMTQSPSSLSASVGDRVTITCRASQNIGGRLVWYQQKPGKAPKLLIYTPSPLQSGVPSRFSGSGSGTDFTLTISSLQPEDFATYYCQQRHYPPFTFGQGTKVEIKR

SEQ ID NO: 458 - protein, 108 aa, Homo sapiens
  VARIANT 3: Xaa = Any Amino Acid
DIXMTQSPSSLSASVGDRVTITCRASQNIGGRLVWYQQKPGKAPKLLIYTPSPLQSGVPSRFSGSGSGTDFTLTISSLQPEDFATYYCQQRHTSPYTFGQGTKVEIKR

SEQ ID NO: 459 - protein, 108 aa, Homo sapiens
  VARIANT 38: Xaa = Any Amino Acid
DIQMTQSPSSLSASVGDRVTITCRASQNIGGRLVWYQXKPGKAPKLLIYTPSPLQSGVPSRFSGSGSGTDFTLTISSLQPEDFATYYCQQRHSEPWTFGQGTKVEIKR

SEQ ID NO: 460 - protein, 108 aa, Homo sapiens
DIQMTQSPSSLSASVGDRVTITCRASQNIGGRLVWYQQKPGKAPKLLIYTPSPLQSGVPSRFSGSGSGTDFTLTISSLQPEDFATYYCQQRSKLPYTFGQGTKVEIKR

SEQ ID NO: 461 - protein, 108 aa, Homo sapiens
DIQMTQSPSSLSASVGDRVTITCRASQNIGGRLVWYQQKPGKAPKLLIYTPSPLQSGVPSRFSGSGSGTDFTLTISSLQPEDFATYYCQQKFKQPYTFGQGTKVEIKR

SEQ ID NO: 462 - protein, 108 aa, Homo sapiens
DIQMTQSPSSLSASVGDRVTITCRASQNIGGRLVWYQQKPGKAPKLLIYTPSPLQSGVPSRFSGSGSGTDFTLTISSLQPEDFATYYCQQRFSSPFTFGQGTKVEIKR

SEQ ID NO: 463 - protein, 108 aa, Homo sapiens
  VARIANT 3, 56, 82: Xaa = Any Amino Acid
DIXMTQSPSSLSASVGDRVTITCRASQNIGGRLVWYQQKPGKAPKLLIYTPSPLQXGVPSRFSGSGSGTDFTLTISSLQPEXFATYYCQQRAVTPFTFGQGTKVEIKR

SEQ ID NO: 464 - protein, 108 aa, Homo sapiens
  VARIANT 27: Xaa = Any Amino Acid
DIQMTQSPSSLSASVGDRVTITCRASXNIGGRLVWYQQKPGKAPKLLIYTPSPLQSGVPSRFSGSGSGTDFTLTISSLQPEDFATYYCQQRATQPYTFGQGTKVEIKR

SEQ ID NO: 465 - protein, 108 aa, Homo sapiens
DIQMTQSPSSLSASVGDRVTITCRASQNIGGRLVWYQQKPGKAPKLLIYTPSPLQSGVPSRFSGSGSGTDFTLTISSLQPEDFATYYCQQRKAPPYTFGQGTKVEIKR

SEQ ID NO: 466 - protein, 108 aa, Homo sapiens
DIQMTQSPSSLSASVGDRVTITCQASQNIGVLLNWYQQKPGKAPKLLIYASSRLQSGVPSRFSGSGSGTDFTLTISSLQPEDFATYYCQQRNFPPPTFGQGTKVEIKR

SEQ ID NO: 467 - protein, 108 aa, Homo sapiens; identical to SEQ ID NO: 400

SEQ ID NO: 468 - protein, 108 aa, Homo sapiens
  VARIANT 3: Xaa = Any Amino Acid
DIXMTQSPSSLSASVGDRVTITCRASQNIHGMLRWYQQKPGKAPKLLIYTPSPSQSGVPSRFSGSGSGTDFTLTISSLQPEDFATYYCQQTATWPFTFGQGTKVEIKR

SEQ ID NO: 469 - protein, 108 aa, Homo sapiens; identical to SEQ ID NO: 410

SEQ ID NO: 470 - protein, 108 aa, Homo sapiens
DIQMTQSPSSLSASVGDRVTITCRASQDIGNMLAWYQQKPGKAPKPLIYYASYLQSGVPSRFSGSGSGTDFTLTISSLQPEDFATYYCQQMRDYPVTFGQGTKVEIKR

SEQ ID NO: 471 - protein, 108 aa, Homo sapiens
DIQMTQSPSSLSASVGDRVTITCRASQDIGNMLAWYQQKPGKAPKPLIYYASYLQSGVPSRFSGSGSGTDFTLTISSLQPEDFATYYCQQMRNLPRTFGQGTKVEIKR

SEQ ID NO: 472 - protein, 108 aa, Homo sapiens; identical to SEQ ID NO: 389

SEQ ID NO: 473 - protein, 108 aa, Homo sapiens
DIQMTQSPSSLSASVGDRVTITCRASQSIGPWLSWYQQKPGKAPKLLFYQVSRLQSGVPSRFSGSGSGTDFTLTIISLQPEDFATYYCQQNLAPPYTFGQGTKVEIKR

SEQ ID NO: 474 - protein, 108 aa, Homo sapiens
DIQMTQSPSSLSASVGDRVTITCRASQSIGPWLSWYQQKPGKAPKLLFYQVSRLPSGVPSRFSGSGSGTDFTVTISSLQPEDFATYYCQQNLAPPYTFGQGTKVEIKR

SEQ ID NO: 475 - protein, 108 aa, Homo sapiens
DIQMTQSPSSLSASVGDRVTITCRASQSIGPWLSWYQQKPGKAPKLLFYQVSRLRSGVPSRFSGSGSGTDFTLTISSLQPEDFASYYCQQNLAPPYTFGQGTKVEIKR

SEQ ID NO: 476 - protein, 108 aa, Homo sapiens
DIQMTQSPSSLSASVGDRVTITCRASQSIGPWLSWYQQKPGKAPKLLIYQVSRLQSGVPSRFSVSGSGTDFTLTISSLQPEDFATYYCQQNLAPPYTFGQGTKVEIKR

SEQ ID NO: 477 - protein, 108 aa, Homo sapiens
DIQMTQSPSSLSASVGDRVTITCRASQPIGSLLEWYQQKPGKAPKLLIYNVSRLQSGVPSRFSGSGSGTDFTLTISSLQPEDFATYYCQQRRFAPRTFGQGTKVEIKR

SEQ ID NO: 478 - protein, 108 aa, Homo sapiens
DIQMTQSPSSLSASVGDRVTITCRASQHITDQLRWYQKKPGKAPKLLIYSASILQSGVPSRFSGSGSGTDFTLTISSLQPEDFATYYCQQIYIRPGTFGQGTKVEIKR

SEQ ID NO: 479 - protein, 108 aa, Homo sapiens; identical to SEQ ID NO: 478

SEQ ID NO: 480 - protein, 108 aa, Homo sapiens
DIQMTQSPSSLSASVGDRVTITCRASQEIGQWLNWYQQKPGKAPKLLIYWGSELQSGVPSRFSGSGSGTDFTLTISSLQPEDFATYYCQQTSRRPFTFGQGTKVEIKR

SEQ ID NO: 481 - protein, 108 aa, Homo sapiens
DIQMTQSPSSLSASVGDRVTITCRASQNIDLELSWYQQKPGKAPKLLIYFTSVLQSGVPSRFSGSGSGTDFTLTISSLQPEDYATYYCQQRIRRPYTFGQGTKVEIKR

SEQ ID NO: 482 - protein, 108 aa, Homo sapiens
DIQMTQSPSSLSASVGDRVTITCRASQNIIDYLNWYQQKPGKAPKLLIYWGSLLQSGVPSRFSGSGSGTDFTLTISSLQPEDFATYYCQQTYRRPFTFGQGTKVEIKR

SEQ ID NO: 483 - protein, 108 aa, Homo sapiens
DIQMTQSPSSLSASVGDRVTITCRASQPIDEWLVWYQQKPGKAPKLLIYRGSLLQSGVPSRFSGSGSGTDFTLTISSLQPEDFATYYCQQYRQMPATFGQGTKVEIKR

SEQ ID NO: 484 - protein, 108 aa, Homo sapiens
DIQMTQSPSSLSASVGDRVTITCRASQPIASRLLWYQQKPGKAPKLLIYYGSVLQSGVPSRFSGSGSGTDFTLTISSLQPEDFATYYCQQTWAHPITFGQGTKVEIKR

SEQ ID NO: 485 - protein, 108 aa, Homo sapiens
DIQMTQSPSSLSASVGDRVTITCRASQPIYKMLRWYQQKPGEAPKLLIYQASNLQSGVPSRFSGSGSGTDFTLTISGLQPEDLATYYCQQFAKWPYTFGQGTKVEIKR

SEQ ID NO: 486 - protein, 108 aa, Homo sapiens
DIQMTQSPSSLSASVGDRVTITCRASQPINTSLNWYQQKPGKAPKLLIYGGSWLQSGVPSRFSGSGSGTDFTLTISSLQPEDFATYYCQQYLYSPSTFGQGTKVEIKR

SEQ ID NO: 487 - protein, 108 aa, Homo sapiens
DIQMTQSPSSLSASVGDRVTITCRASQPIHENLDWYQQKPGKAPKLLIYGASMLQSGVPSRFSGSGSGTDFTLTISSLQPEDSATYYCQQGWVYPQTFGQGTKVEIKR

SEQ ID NO: 488 - protein, 108 aa, Homo sapiens
DIQMTQSPSSLSASVGDRVTITCRASQPIDTFLRWYQQKPGKAPKLLIYRASQLQSGVPSRFSGSGSGTDFTLTISSLQPEDFATYYCQQWARSPFTFGQGTKVKIKR

SEQ ID NO: 489 - protein, 108 aa, Homo sapiens
DIQMTQSPSSLSASVGDRVTITCRASQFIEWYLAWYQQKPGKAPKLLIYNGSVLQSGVPSRFSGSGSGTDFTLTISSLQPEDFATYYCQQRVARPFTFGQGTKVEIKR

SEQ ID NO: 490 - protein, 108 aa, Homo sapiens
DIQMTQSPSSLSASVGDRVTITCRASQYIGTALDWYQQKPGKAPKLLIYAVSLLQSGVPSRFSGSGSGTDFTLTISSLQPEDLATYYCQQAFAPPMTFGQGTKVEIKR

SEQ ID NO: 491 - protein, 108 aa, Homo sapiens
DIQMTQSPSSLSASVGDRVTITCRASQHIGDYLAWYQQKPGKAPKLLIYPSSQLQSGVPSRFSGSGSGTDFTLTISSLQPEDFATYYCQQRRYLPMTFGQGTKVEIKR

SEQ ID NO: 492 - protein, 108 aa, Homo sapiens
DIQMTQSPSSLSASVGDRVTITCRASQDIGEYLQWYQQKPGKAPKLLIYWTSMLQSGVPSRFSGSGSGTDFTLTISSLQPEDFATYYCQQEARTPFTFGQGTKVEIKR

SEQ ID NO: 493 - protein, 108 aa, Homo sapiens
DIQMTQSPSSLSASVGDRVTITCRASQDINDYLSWYQQKPGKAPKLLIYWGSSLQSGVPSRFSGSGSGTDFTLTISSLQPEDFATYYCQQRAYRPYTFGQGTKVEIKR

SEQ ID NO: 494 - protein, 108 aa, Homo sapiens
DIQMTQSPSSLSASVGDRVTITCRASQDIFPFLNWYQQKPGKAPELLIYRASILHSGVPSRFSGSGSGTDFTLTISSLQPEDFATYYCQQIARSPRTFGQGTKVEIKR

SEQ ID NO: 495 - protein, 108 aa, Homo sapiens
DIQMTQSPSSLSASVGDRVTITCRASQDIEDWLAWYQQKPGKAPKLLIYWGSTLQSGVPSRFSGSGSGTDFTLTISSLQPEDFATYYCQQSKGTPYTFGQGTKVEIKR

SEQ ID NO: 496 - protein, 108 aa, Homo sapiens
DIQMTQSPSSLSASVGDRVTITCRASQDIDDWLHWYQQKPGKAPKLLIYWSSSLQSGVPSRFSGSGSGTDFTLTISSLQPEDFATYYCQQEKYRPFTFGQGTKVEIKR

SEQ ID NO: 497 - protein, 108 aa, Homo sapiens
DIQMTQSPSSLSASVGDRVTITCRASQDIQTWLSWYQQKPGKAPKLLIYHSSYLQSGVPSRFSGSGSGTDFTLTISSLQPEDFATYYCQQYDTLPGTFGQGTKVEIKR

SEQ ID NO: 498 - protein, 108 aa, Homo sapiens
  VARIANT 28: Xaa = Any Amino Acid
DIQMTQSPSSLSASVGDRVTITCRASQXISGCLYWYQQKPGKAPKLLIYRGSHLQSGVPSRFSGSGSGTDFTLTISSLQPEDFATYYCQQDCDPPSTFGQGTKVEIKR

SEQ ID NO: 499 - protein, 108 aa, Homo sapiens
DIQMTQSPSSLSASVGDRVTITCRASQSIEKKLVWYQQKPGKAPKLLIYYTSYLQSGVPSRFSGSGSGTDFTLTISSLQPEDFATYYCQQYQGHPLTFGQGTKVEIKR

SEQ ID NO: 500 - protein, 108 aa, Homo sapiens
DIQMTQSPSSLSASVGDRVTITCRASQPIGDMLMWYQQKPGKAPKLLIYGGSNLQSGVPSRFSGSGSGTDFTLTISSLQPEDFATYYCQQRRLAPSTFGQGTKVEIKR

SEQ ID NO: 501 - protein, 108 aa, Homo sapiens
DIQMTQSPSSLSASVGDRVTITCRASQPIDERLNWYQQKPGKAPKLLIYRRSWLQSGVPSRFSGSGSGTDFTLTISSLQPEDFATYYCQQWGHHPSTFGQGTKVEIKR

SEQ ID NO: 502 - protein, 108 aa, Homo sapiens
DIQMTQSPSSLSASVGDRVTITCRASQPIDSRLMWYQQKPGKAPKLLIYFASYLQSGVPSRFSGSGSGTDFTLTISSLQPEDFATYYCQQYLMHPLTFGQGTKVEIKR

SEQ ID NO: 503 - protein, 108 aa, Homo sapiens
DIQMTQSPSSLSASVGDRVTITCRASQPIHYALDWYQQKPGKAPKLLIYSTSILQSGVPSRFSGSGSGTDFTLTISSLQPEDFATYYCQQWFRWPTTFGQGTKVEIKR

SEQ ID NO: 504 - protein, 108 aa, Homo sapiens
DIQMTQSPSSLSASVGDRVTITCRASQPIGDFLLWYQQKPGKAPKLLIYGASTLQSGVPSRFSGSGSGTDFTLTISSLQPEDFATYYCQQRRFFPSTFGQGTKVEIKR

SEQ ID NO: 505 - protein, 108 aa, Homo sapiens
DIQMTQSPSSLSASVGDRVTITCRASQHIGQNLNWYQQKPGKAPKLLIYWGSDLQSGVPSRFSGSGSGTDFTLTISSLQPEDFATYYCQQLRFPPLTFGQGTKVEIKR

SEQ ID NO: 506 - protein, 108 aa, Homo sapiens
DIQMTQSPSSLSASVGDRVTITCRASQDIGEYLYWYQQKPGKAPKLLIYMISNLQSGVPSRFSGSGSGTDFTLTISSLQPEDFATYYCQQLVAWPYTFGQGTKVEIKR

SEQ ID NO: 507 - protein, 108 aa, Homo sapiens
DIQMTQSPSSLSASVGDRVTITCRASQDIYGELSWYQQKPGKAPKLLIYFSSILQSGVPSRFSGSGSGTDFTLTISSLQPEDFATYYCQQRSVRPYTFGQGTKVEIKR

SEQ ID NO: 508 - protein, 108 aa, Homo sapiens
DIQMTQSPSSLSASVGDRVTITCRASQDIHGYLDWYQQKPGKAPKLLIYYASYLQSGVPSRFSGSGSGTDFTLTISSLQPEDFATYYCQQRYQHPVTFGQGTKVEIKR

SEQ ID NO: 509 - protein, 108 aa, Homo sapiens
DIQMTQSPSSLSASVGDRVTITCRASQDINSRLSWYQQKPGKAPKLLIYYASYLRSGVPSRFSGSGSGTDFTLTISSLQPEDFATYYCQQWWSHPITFGQGTKVEIKR

SEQ ID NO: 510 - protein, 108 aa, Homo sapiens
DIQLTQSPSSLSASVGDRVTITCRASQDIGDHLLWYQQKPGKAPKLLIYGASQLQSGVPSRFSGSGSGTDFTLTISSLQPEDFATYYCQQVRIYPRTFGQGTKVEIKR

SEQ ID NO: 511 - protein, 108 aa, Homo sapiens
DIQMTQSPSSLSASVGDRVTITCRASQDIDRWLRWYQQKPGKAPRLLIYWTSELQSGVPSRFSGSGSGTDFTLTISSLQPEDFATYYCQQEFRMPVTFGQGTKVEIKR

SEQ ID NO: 512 - protein, 108 aa, Homo sapiens
DIQMTQSPSSLSASVGDRVTITCRASQDIGDHLLWYQQKPGKAPKLLIYGSSALQSGVPSRFSGSGSGTDFTLTISSLQPEDFATYYCQQVRGFPSTFGQGTKVEIKR

SEQ ID NO: 513 - protein, 108 aa, Homo sapiens
DIQMTQSPSSLSASVGDRVTITCRASQDISDYLSWYQQKPGKAPKLLIYWTSMLQSGVPSRFSGSRSGTDFTLTISSLQPEDFATYYCQQRYRRPFTFGQGTKVEIKR

SEQ ID NO: 514 - protein, 108 aa, Homo sapiens
DIQMTQSPSSLSASVGDRVTITCRASQSIGKHLAWYQQKPGKAPKLLIYRASLLQSGVPSRFSGSGSGTDFTLTISSLQPEDFATYYCQQHSRSPRTFGQGTKVEIKR

SEQ ID NO: 515 - protein, 108 aa, Homo sapiens
DIQMTQSPSSLSASVGDRVTITCRASQFIGLHLVWYQQKPGKASKLLIYNTSDLQSGVPSRFSGSGSGTDFTLTISSLQPEDFATYYCQQMAHYPYTFSQGTKVEIKR

SEQ ID NO: 516 - protein, 108 aa, Homo sapiens
DIQMTQSPSSLSASVGDRVTITCRASQSIGDMLLWYQQKPGKAPKLLIYGSSALQSGVPSRFSGSGSGTDFTLTISSLQPEDFATYYCQQVRTYPSTFGQGTKVEIKR

SEQ ID NO: 517 - protein, 108 aa, Homo sapiens
DIQMTQSPSSLSASVGDRVTITCRASRLIGKHLSWYQQKPGKAPKLLIYRSSVLQSGVPSRFSGSGSGTDFTLTISSLQPEDFATYYCQQHATSPRTFGQGTKVEIKR

SEQ ID NO: 518 - protein, 108 aa, Homo sapiens
DIQMTQSPSSLSASVGDRVTITCRASQYIDKRLLWYQQKPGEAPKLLIYYASYLQSGVPSRFSGSGSGTDFTLTISSLQPEDFATYYCQQQFIHPLTFGQGTKVEIKR

SEQ ID NO: 519 - protein, 108 aa, Homo sapiens
DIQMTQSPSSLSASVGDRVTITCRASQYIGQMLNWYQQKPGKAPKLLIYQASGLQSGVPSRFSGSGSGTDFTLTISSLQPEDFATYYCQQSYVHPYTFGQGTKVEIKR

SEQ ID NO: 520 - protein, 108 aa, Homo sapiens
DIQMTQSPSSLSASVGDRVTITCRASQAIGNWLDWYQQKPGKAPKLLIYWGSELQSGVPSRFSGSGSGTDFTLTISSLQPEDFATYYCQQRSSSPFTFGQGTKVEIKR

SEQ ID NO: 521 - protein, 108 aa, Homo sapiens
DIQMTQSPSSLSASVGDRVTITCRASQAIDMYLTWYQQKPGKAPRLLIYWASISQSGVPSRFSGSGSGTDYTLTISSLQPEDFATYYCQQRKARPYTFGQGTKVEIKR

SEQ ID NO: 522 - protein, 108 aa, Homo sapiens
DIQMTQSPSSLSASVGDRVTITCRASQAIEWYLAWYQQKPGKAPKLLIYNASILQSGVPSRFSGSGSGTDFTLTISSLQPEDFATYYCQQRAFSPLTFGQGTKVEIKR

SEQ ID NO: 523 - protein, 108 aa, Homo sapiens
DIQMTQSPSSLSASVGDRVTITCRASQAIWTYLNWYQQKPGKAPKLLIYGASQLQSGVPSRFSGSGSGTDFTLTISSLQPEDFATYYCQQTESFPVTFGQGTKVEIKR

SEQ ID NO: 524 - protein, 108 aa, Homo sapiens
DIQMTQSPSSLSASVGDRVTITCRASQTITDYLNWYQQKPGKAPKLLIYWGSILQSGVPSRFSGSGSGTDFTLTISSLQPEDFATYYCQQSAHRPYTFGQGTKVEIKR

SEQ ID NO: 525 - protein, 108 aa, Homo sapiens
DIQMTQSPSSLSASVGDRVTITCRASQKIGSHLSWYQQKPGKAPKLLIYRTSQLQSGAPSRFSGSGSGTDFTLTISSLQPEDFATYYCQQQAKSPRTFGQGTKVEIKR

SEQ ID NO: 526 - protein, 108 aa, Homo sapiens
DIQMTQSPSSLSASVGDRVTITCRASQQIDDYLNWYQQKPGKAPKLLIYWTSLLQSGVPSRFSGSGSGTDFTLTISSLQPEDFATYYCQQSAHRPFTFGQGTKVEIKR

SEQ ID NO: 527 - protein, 108 aa, Homo sapiens
DIQMTQSPSSLSASVGDRVTITCRASQQIDDRLSWYQQKPGKAPKLLIYFKSFLQSGVPSRFSGSGSGTDFTLTISSLQPEDFATYYCQQYQAHPLTFGQGTKVEIKR

SEQ ID NO: 528 - protein, 108 aa, Homo sapiens
DIQMTQSPSSLSASVGDRVTITCRASQRIAGCLSWYQQKPGKAPKLLIYRTSLLQSGVPSRFSGSGSGTDFTLTISSLQPEDFATYYCQQDCTFPRTFGQGTKVEIKR

SEQ ID NO: 529 - protein, 108 aa, Homo sapiens
DIQMTQSPSSLSASVGDRVTITCRASQRISGCLYWYQQKPGKAPKLLIYRGSHLQSGVPSRFSGSGSGTDFTLTISSLQPEDFATYYCQQDCDPPSTFGQGTKVEIKR

SEQ ID NO: 530 - protein, 108 aa, Homo sapiens; identical to SEQ ID NO: 519

SEQ ID NO: 531 - protein, 108 aa, Homo sapiens
DIQMTQSPSSLSASVGDRVTITCRASQNISYHLVWYQQKPGKAPKLLIYSSSNLQSGVPSRFSGSGSGTDFTLTISSLQPEDLATYYCQQLASWPHTLGQGTKVEIKR

SEQ ID NO: 532 - protein, 108 aa, Homo sapiens
DIQMTQSPSSLSASVGDRVTITCRASQNISRGLRWYQQKPGKAPKLLIYHASKLQSGVPSRFSGSGSGTDFTLTISSLQPEDFATYYCQQYKVFPGTFGQGTKVEIKR

SEQ ID NO: 533 - protein, 108 aa, Homo sapiens
DIQMTQSPSSLSASVGDRVTITCRASQNIGSHLLWYQQKPGKAPKLLIYGSSSLQSGVPSRFSGSGSGTDFTLTISSLQPEDFATYYCQQVRLAPHTFGQGTKVEIKR

SEQ ID NO: 534 - protein, 108 aa, Homo sapiens
DIQMTQSPSSLSASVGDRVTITCRASQNIGMYLKWYQQKPGKAPKLLIYYSSSLQSGVPSRFSGSGSGTDFTLTISSLQPEDFATYYCQQNRMRPTTFGQGTKVEIKR

SEQ ID NO: 535 - protein, 108 aa, Homo sapiens
DIQMTQSPSSLSASVGDRVTITCRASQGIDWYLSWYQQKPGKAPKLLIYEGSNLQSGVPSRFSGSGSGTDFTLTISSLQPEDSATYYCQQRAAYPFTFGQGTKVEIKR

SEQ ID NO: 536 - protein, 108 aa, Homo sapiens
DIQMTQSPSSLSASVGDRVTITCRASQGIGVALDWYQQKPGKAPKLLIYMASRLQSGVPSRFSGSGSGTDFTLTISSLQPEDFATYYCQQYSELPVTFGQGTKVEIKR

SEQ ID NO: 537 - protein, 108 aa, Homo sapiens
DIQMTQSPSSLSASVGDRVTITCRASQEISGELTWYQQKPGKAPKLLIYFSSILQSGVPSRFSGSGSGTDFTLTISSLQPEDFATYYCQQRKLRPYTFGQGTKVEIKR

SEQ ID NO: 538 - protein, 108 aa, Homo sapiens
DIQMTQSPSSLSASVGDRVTITCRASQEIGQWLNWYQQKPGKAPKLLIYWGSELQSGVPSRFSGSGSGTDFTLTISSLQPEDFATYYCQQTQLRPSTFGQGTKVEIKR

SEQ ID NO: 539 - protein, 108 aa, Homo sapiens
DIQMTQSPSSLSASVGDRVTITCRASQEIGQWLNWYQQKPGKAPKLLIYWGSELQSGVPSRFSGSGSGTDFTLTISSLQPEDFATYYCQQVSRNPFTFGQGTKVEIKR

SEQ ID NO: 540 - protein, 108 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPNTFGQGTKVEIKR

SEQ ID NO: 541 - protein, 108 aa, Homo sapiens
DIQMTQSPSSLSASVGDRVTITCRASQSIIKHLKWYQQKPGKAPKLLIYGASRLQSGVPSRFSGSGSGTDFTLTISSLQPEDFATYYCQQGARWPQTFGQGTKVEIKR

SEQ ID NO: 542 - protein, 108 aa, Homo sapiens
DIQMTQSPSSLSASVGDRVTITCRASQSIFRHLKWYQQKPGKAPKLLIYAASRLQSGVPSRFSGSGSGTDFTLTISSLQPEDFATYYCQQVALYPKTFGQGTKVEIKR

SEQ ID NO: 543 - protein, 108 aa, Homo sapiens
DIQMTQSPSSLSASVGDRVTITCRASQSIYYHLKWYQQKPGKAPKLLIYKASTLQSGVPSRFSGSGSGTDFTLTISSLQPEDFATYYCQQVRKVPRTFGQGTKVEIKR

SEQ ID NO: 544 - protein, 108 aa, Homo sapiens
DIQTTQSPSSLSASVGDRVTITCRASQYIGRYLRWYQQKPGKAPKLLIYDSSVLQSGVPSRFSGSGSGTDFTLTISSLQPEDFATYYCQQRYRMPYTFGQGTRVEIKR

SEQ ID NO: 545 - protein, 108 aa, Homo sapiens
DIQMTQSPSSLSASVGDRVTITCRASQYIGRYLRWYQQKPGKAPKLLIYDSSVLQSGVPSRFSGSGSGTDFTLTISSLQPEDFATYYCQQRYMQPFTFGQGTKVEIKR

SEQ ID NO: 546 - protein, 108 aa, Homo sapiens
DIQMTQSPSSLSASVGDRVTITCRASQWIGRYLRWYQQKPGKAPKLLIYNGSQLQSGVPSRFSGSGSGTDFTLTISSLQPEDFATYYCQQRYLQPYTFGQGTKVEIKR

SEQ ID NO: 547 - protein, 108 aa, Homo sapiens
DIQMTQSPSSLSASVGDRVTITCRASQYISRQLRWYQQKPGKAPRLLIYGASVLQSGIPSRFSGSGSGTDFTLTISSLQPEDFATYYCQQRYITPYTFGQGTKVEVKR

SEQ ID NO: 548 - protein, 108 aa, Homo sapiens
DIQMTQSPSSLSASVGDRVTITCRASQYIGRYLRWYQQKPGKAPKLLIYDSSVLQSGVPSRFSGSGSGTDFTLTISSLQPEDFATYYCQQRYSSPYTFGQGTKVEIKR

SEQ ID NO: 549 - protein, 108 aa, Homo sapiens
DIQMTQSPSSLSASVGDRVTITCRASQWIHRQLKWYQQKPGKAPKLLIYYASILQSGVPSRFSGSGSGTDFTLTISSLQPEDFATYYCQQTFSKPSTFGQGTKVEIKR

SEQ ID NO: 550 - protein, 108 aa, Homo sapiens
DIQMTQSPSSLSASVGDRVTITCRASQKIATYLNWYQQKPGKAPKLLIYRSSSLQSAVPSRFSGSGSGTVFTLTISSLQPEDFATYYCQQTYAVPPTFGQGTKVEIKR

SEQ ID NO: 551 - protein, 108 aa, Homo sapiens
DIQMTQSPSSLSASVGDRVTITCRASQWIDTGLAWYQQKPGKAPRLLIYNVSRLQSGVPSRFSGSGSGTDFTLTISSLQPEDFATYYCQQYWGSPTTFGQGTKVEIKR

SEQ ID NO: 552 - protein, 108 aa, Homo sapiens
DIQMTQSPSSLSASVGDRVTITCRASQEIYSWLAWYQQRPGKAPKLLIYNASHLQSGVPSRFSGSGSGTDFTLTISSLQPEDFATYYCQQVIGDPVTFGQGTKVEIKR

SEQ ID NO: 553 - protein, 108 aa, Homo sapiens
DIQMTQSPSSLSASVGDRVTITCRASQSISSYLNWYQQKPGKAPTLLIYRLSVLQSGVPSRFSGSGSGTDFTLTISSLQPEDFATYYCQQTYNVPPTFGQGTKVEIKR

SEQ ID NO: 554 - protein, 108 aa, Homo sapiens
DIQMTQSPSSLSASVGDRVTITCRASQSISSYLNWYQQKPGKAPKLLIYRNSFLQSGVPSRFSGSGSGTDFTLTISSLQPEDFATYYCQQTYTVPPTFGQGTKVEIKQ

SEQ ID NO: 555 - protein, 108 aa, Homo sapiens
DIQMTQSPSSLSASVGDRVTITCRASQSISSYLNWYQQKPGKAPKLLIYRNSQLQSGVPSRFSGSGSGTDFTLTISSLQPEDFATYYCQQTFAVPPTFGQGTKVEIKR

SEQ ID NO: 556 - protein, 35 aa, Homo sapiens
  VARIANT 30, 31: Xaa = Any Amino Acid
EVQLLESGGGLVQPGGSLRLSCAASGFTFXXYNMS

SEQ ID NO: 557 - protein, 123 aa, Homo sapiens
EVQLLESGGGLVQPGGSLRLSCAASGFTFSKYWMSWVRQAPGKGLEWVSSIDFMGPHTYYADSVKGRFTISRDNSKNTLYLQMNSLRAEDTAVYYCAKGRTSMLPMKGKFDYWGQGTLVTVSS

SEQ ID NO: 558 - protein, 118 aa, Homo sapiens
EVQLLESGGGLVQPGGSLRLSCAASGFTFYDYNMSWVRQAPGKGLEWVSTITHTGGVTYYADSVKGRFTISRDNSKNTLYLQMNSLRAEDTAVYYCAKQNPSYQFDYWGQGTLVTVSS

SEQ ID NO: 559 - protein, 118 aa, Homo sapiens
EVQLLESGGGLVQPGGSLRLSCAASGFTFHRYSMSWVRQAPGKGLEWVSTILPGGDVTYYADSVKGRFTISRDNSKNTLYLQMNSLRAEDTAVYYCAKQTPDYMFDYWGQGTLVTVSS

SEQ ID NO: 560 - protein, 117 aa, Homo sapiens
EVQLLESGGGLVQPGGSLRLSCAASGFTFWKYNMAWVRQAPGKGLEWVSTILGEGNNTYYADSVKGRFTISRDNSKNTLYLQMNSLRAEDTAVYYCAKTMDYKFDYWGQGTLVTVSS

SEQ ID NO: 561 - protein, 118 aa, Homo sapiens
EVQLLESGGGLVQPGGSLRLSCTASGFTFDEYNMSWVRQAPGKGLEWVSTILPHGDRTYYADSVKGRFTISRDNSKNTLYLQMNSLRAEDTAVYYCAKQDPLYRFDYWGQGTLVTVSS

SEQ ID NO: 562 - protein, 120 aa, Homo sapiens
EVQLLESGGGLVQPGGSLRLSCAASGFTFDLYDMSWVRQAPGKGLEWVSSIVNSGVRTYYADSVKGRFTISRDNSKNTLYLQMNSLRAEDTAVYYCAKLNQSYHWDFDYWGQGTLVTVSS

SEQ ID NO: 563 - protein, 118 aa, Homo sapiens
EVQLLESGGGLVQPGGSLRLSCAASGFTFSDYRMSWVRQAPGKGLEWVSTIISNGKFTYYADSVKGRFTISRDNSKNTLYLQMNSLRAEDTAVYYCAKQDWMYMFDYWGQGTLVTVSS

SEQ ID NO: 564 - protein, 108 aa, Homo sapiens
DIQMTQSPSSLSASVGDRVTITCRASQSISSYLNWYQQKPGKAPKLLIYRNSPLQSGVPSRFSGSGSGTDFTLTISSLQPEDFATYYCQQTYRVPPTFGQGTKVEIKR

SEQ ID NO: 565 - protein, 108 aa, Homo sapiens
DIQMTQSPSSLSASVGDRVTITCRASQHIHRELRWYQQKPGKAPKLLIYQASRLQSGVPSRFSGSGSGTDFTLTISSLQPEDFATYYCQQKYLPPYTFGQGTKVEIKR

SEQ ID NO: 566 - protein, 108 aa, Homo sapiens
DIQMTQSPSSLSASVGDRVTITCRASQHIHRELRWYQQKPGKAPKLLIYQASRLQSGVPSRFSGSGSGTDFTLTISSLQPEDFATYYCQQRYRVPYTFGQGTKVEIKR

SEQ ID NO: 567 - protein, 108 aa, Homo sapiens
DIQMTQSPSSLSASVGDRVTITCRASQSIGRRLKWYQQKPGAAPRLLIYRTSWLQSGVPSRFSGSGSGTDFTLTISSLQPEDFATYYCQQTSQWPHTFGQGTKVEIKR

SEQ ID NO: 568 - protein, 108 aa, Homo sapiens
DIQMTQSPSSLSASVGDRVTITCRASQKIYKNLRWYQQKPGKAPKLLIYNSSILQSGVPSRFSGSGSGTDFTLTISSLQPEDFATYYCQQRYLSPYTFGQGTKVEIKR

SEQ ID NO: 569 - protein, 108 aa, Homo sapiens
DIQMTQSPSSLSASVGDRVTITCRASQKIYNNLRWYQQKPGKAPKLLIYNTSILQSGVPSRFSGSGSGTDFTLTISSLQPEDFATYYCQQRWRAPYTFGQGTKVEIKR

SEQ ID NO: 570 - protein, 108 aa, Homo sapiens
DIQMTQSPSSLSASVGDRVTITCRASQWIYKSLGWYQQKPGKAPKLLIYQSSLLQSGVPSRFSGSGSGTDFTLTISSLQPEDFATYYCQQYHQMPRTFGQGTKVEIKR

SEQ ID NO: 571 - protein, 108 aa, Homo sapiens
DIQMTQSPSSLSASVGDRVTITCRASQWIYRHLRWYQQKPGKAPKLLIYDASRLQSGVPTRFSGSGSGTDFTLTISSLQPEDFATYYCQQTHNPPKTFGQGTKVEIKR

SEQ ID NO: 572 - protein, 116 aa, Homo sapiens
EVQLLESGGGLVQPGGSLRLSCAASGFTFWPYTMSWVRQAPGKGLEWVSTISPFGSTTYYADSVKGRFTISRDNSKNTLYLQMNSLRAEDTAVYYCAKGGKDFDYWGQGTLVTVSS

SEQ ID NO: 573 - protein, 117 aa, Homo sapiens
EVQLLESGGGLVQPGGSLRLSCAASGFTFWPYTMSWVRQAPGKGLEWVSTISPFGSTTYYADSVKGRFTISRDNSKNTLYLQMNSLRAEDTAVYYCAKGNLEPFDYWGQGTLVTVSS

SEQ ID NO: 574 - protein, 117 aa, Homo sapiens
EVQLLESGGGLVQPGGSLRLSCAASGFTFWPYTMSWVRQAPGKGLEWVSTISPFGSTTYYADSVKGRFTISRDNSKNTLYLQMNSLRAEDTAVYYCAKKLSNGFDYWGQGTLVTVSS

SEQ ID NO: 575 - protein, 118 aa, Homo sapiens
EVQLLESGGGLVQPGGSLRLSCAASGFTFWPYTMSWVRQAPGKGLEWVSTISPFGSTTYYADSVKGRFTISRDNSKNTLYLQMNSLRAEDTAVYYCAKVVKDNTFDYWGQGTLVTVSS

SEQ ID NO: 576 - protein, 118 aa, Homo sapiens
EVQLLESGGGLVQPGGSLRLSCAASGFTFWPYTMSWVRQAPGKGLEWVSTISPFGSTTYYADSVKGRFTISRDNSKNTLYLQMNSLRAEDTAVYYCAKNTGGKQFDYWGQGTLVTVSS

SEQ ID NO: 577 - protein, 118 aa, Homo sapiens
EVQLLESGGGLVQPGGSLRLSCAASGFTFWPYTMSWVRQAPGKGLEWVSTISPFGSTTYYADSVKGRFTISRDNSKNTLYLQMNSLRAEDTAVYYCAKKTGPSSFDYWGQGTLVTVSS

SEQ ID NO: 578 - protein, 120 aa, Homo sapiens
EVQLLESGGGLVQPGGSLRLSCAASGFTFWPYTMSWVRQAPGKGLEWVSTISPFGSTTYYADSVKGRFTISRDNSKNTLYLQMNSLRAEDTAVYYCAKRTENRGVSFDYWGQGTLVTVSS

SEQ ID NO: 579 - protein, 122 aa, Homo sapiens
EVQLLESGGGLVQPGGSLRLSCAASGFTFWPYTMSWVRQAPGKGLEWVSTISPFGSTTYYADSVKGRFTISRDNSKNTLYLQMNSLRAEDTAVYYCAKSDVLKTGLDGFDYWGQGTLVTVSS

SEQ ID NO: 580 - protein, 120 aa, Homo sapiens
EVQLLESGGGLVQPGGSLRLSCAASGFTFMAYQMAWVRQAPGKGLEWVSTIHQTGFSTYYADSVKGRFTISRDNSKNTLYLQMNSLRAEDTAVYYCAKVRSMRPYKFDYWGQGTLVTVSS

SEQ ID NO: 581 - protein, 120 aa, Homo sapiens
EVQLLESGGGLVQPGGSLRLSCAASGFTFKDYDMTWVRQAPGKGLEWVSMISSSGLWTYYADSVKGRFTISRDNSKNTLYLQMNSLRAEDTAVYYCAKGFRLFPRTFDYWGQGTLVTVSS

SEQ ID NO: 582 - protein, 121 aa, Homo sapiens
EVQLLESGGGLVQPGGSLRLSCAASGFTFHDYVMGWARQAPGKGLEWVSLIKPNGSPTYYADSVKGRFTISRDNSKNTLYLQMNSLRAEDTAVYYCAKGRGRFNVLQFDYWGQGTLVTVSS

SEQ ID NO: 583 - protein, 118 aa, Homo sapiens
EVQLLESGGGLVQPGGSLRLSCTASGFTFRHYRMGWVRQAPGKGLEWVSWIRPDGTFTYYADSVKGRFTISRDNSKNTLYLQMNSLRAEDTAVYYCAKSYMGDRFDYWGQGTLVTVSS

SEQ ID NO: 584 - protein, 116 aa, Homo sapiens
EVQLLESGGGLVQPGGSLRLSCAASGFTFMWDKMGWVRQAPGKGLEWVSFIGREGYGTYYADSVKGRFTISRDNSKNTLYLQMNSLRAEDTAVYYCAKSVASFDYWGQGTLVTVSS

SEQ ID NO: 585 - protein, 117 aa, Homo sapiens
EVQLLESGGGLVQPGGSLRLSCAASGFTFWAYPMSWVRQAPGKGLEWVSSISSWGTGTYYADSVKGRFTISRDNSKNTLYLQMNSLRAEDTAVYYCAKGGQGSFDYWGQGTLVTVSS

SEQ ID NO: 586 - protein, 115 aa, Camelid
QVQLQESGGGLVQPGGSLRLSCEASGFTFSRFGMTWVRQAPGKGVEWVSGISSLGDSTLYADSVKGRFTISRDNAKNTLYLQMNSLKPEDTAVYYCTIGGSLNPGGQGTQVTVSS

SEQ ID NO: 587 - protein, 115 aa, Camelid
QVQLQESGGGLVQPGNSLRLSCAASGFTFRNFGMSWVRQAPGKEPEWVSSISGSGSNTIYADSVKDRFTISRDNAKSTLYLQMNSLKPEDTAVYYCTIGGSLSRSSQGTQVTVSS

SEQ ID NO: 588 - protein, 114 aa, Camelid
QVQLQESGGGLVQPGGSLRLTCTASGFTFSSFGMSWVRQAPGKGLEWVSAISSDSGTKNYADSVKGRFTISRDNAKKMLFLQMNSLRPEDTAVYYCVIGRGSPSSQGTQVTVSS

SEQ ID NO: 589 - protein, 114 aa, Camelid
QVQLQESGGGLVQPGGSLRLTCTASGFTFRSFGMSWVRQAPGKGLEWVSAISADGSDKRYADSVKGRFTISRDNGKKMLTLDMNSLKPEDTAVYYCVIGRGSPASQGTQVTVSS

SEQ ID NO: 590 - protein, 128 aa, Camelid
AVQLVESGGGLVQAGDSLRLSCVVSGTTFSSAAMGWFRQAPGKEREFVGAIKWSGTSTYYTDSVKGRFTISRDNVKNTVYLQMNNLKPEDTGVYTCAADRDRYRDRMGPMTTTDFRFWGQGTQVTVSS

SEQ ID NO: 591 - protein, 124 aa, Camelid
QVKLEESGGGLVQTGGSLRLSCAASGRTFSSFAMGWFRQAPGREREFVASIGSSGITTNYADSVKGRFTISRDNAKNTVYLQMNSLKPEDTGLCYCAVNRYGIPYRSGTQYQNWGQGTQVTVSS

SEQ ID NO: 592 - protein, 120 aa, Camelid
EVQLEESGGGLVQPGGSLRLSCAASGLTFNDYAMGWYRQAPGKERDMVATISIGGRTYYADSVKGRFTISRDNAKNTVYLQMNSLKPEDTAIYYCVAHRQTVVRGPYLLWGQGTQVTVSS

SEQ ID NO: 593 - protein, 123 aa, Camelid
QVQLVESGGKLVQAGGSLRLSCAASGRTFSNYAMGWFRQAPGKEREFVAGSGRSNSYNYYSDSVKGRFTISRDNAKNTVYLQMNSLKPEDTAVYYCAASTNLWPRDRNLYAYWGQGTQVTVSS

SEQ ID NO: 594 - protein, 125 aa, Camelid
EVQLVESGGGLVQAGDSLRLSCAASGRSLGIYRMGWFRQVPGKEREFVAAISWSGGTTRYLDSVKGRFTISRDSTKNAVYLQMNSLKPEDTAVYYCAVDSSGRLYWTLSTSYDYWGQGTQVTVSS

SEQ ID NO: 595 - protein, 125 aa, Camelid
QVQLVEFGGGLVQAGDSLRLSCAASGRSLGIYKMAWFRQVPGKEREFVAAISWSGGTTRYIDSVKGRFTLSRDNTKNMVYLQMNSLKPDDTAVYYCAVDSSGRLYWTLSTSYDYWGQGTQVTVSS

SEQ ID NO: 596 - protein, 124 aa, Camelid
EVQLVESGGGLVQAGGSLSLSCAASGRTFSPYTMGWFRQAPGKEREFLAGVTWSGSSTFYGDSVKGRFTASRDSAKNTVTLEMNSLNPEDTAVYYCAAAYGGGLYRDPRSYDYWGRGTQVTVSS

SEQ ID NO: 597 - protein, 131 aa, Camelid
AVQLVESGGGLVQAGGSLRLSCAASGFTLDAWPIAWFRQAPGKEREGVSCIRDGTTYYADSVKGRFTISSDNANNTVYLQTNSLKPEDTAVYYCAAPSGPATGSSHTFGIYWNLRDDYDNWGQGTQVTVSS

SEQ ID NO: 598 - protein, 126 aa, Camelid
EVQLVESGGGLVQAGGSLRLSCAASGFTFDHYTIGWFRQVPGKEREGVSCISSSDGSTYYADSVKGRFTISSDNAKNTVYLQMNTLEPDDTAVYYCAAGGLLLRVEELQASDYDYWGQGIQVTVSS

SEQ ID NO: 599 - protein, 128 aa, Camelid
AVQLVDSGGGLVQPGGSLRLSCTASGFTLDYYAIGWFRQAPGKEREGVACISNSDGSTYYGDSVKGRFTISRDNAKTTVYLQMNSLKPEDTAVYYCATADRHYSASHHPFADFAFNSWGQGTQVTVSS

SEQ ID NO: 600 - protein, 120 aa, Camelid
EVQLVESGGGLVQAGGSLRLSCAAYGLTFWRAAMAWFRRAPGKERELVVARNWGDGSTRYADSVKGRFTISRDNAKNTVYLQMNSLKPEDTAVYYCAAVRTYGSATYDIWGQGTQVTVSS

SEQ ID NO: 601 - protein, 123 aa, Camelid
EVQLVESGGGLVQDGGSLRLSCIFSGRTFANYAMGWFRQAPGKEREFVAAINRNGGTTNYADALKGRFTISRDNTKNTAFLQMNSLKPDDTAVYYCAAREWPFSTIPSGWRYWGQGTQVTVSS

SEQ ID NO: 602 - protein, 125 aa, Camelid
DVQLVESGGGWVQPGGSLRLSCAASGPTASSHAIGWFRQAPGKEREFVVGINRGGVTRDYADSVKGRFAVSRDNVKNTVYLQMNRLKPEDSAIYICAARPEYSFTAMSKGDMDYWGKGTLVTVSS

SEQ ID NO: 603 - DNA, 324 bp, Homo sapiens
gacatccagatgacccagtctccatcctccctgtctgcatctgtaggagaccgtgtcaccatcacttgccgggcaagtcacaatattgattctcgtttaagttggtaccagcagaaaccagggaaagcccctaagctcctgatctatagggcgtccgttttgcaaagtggggtcccttcacgtttcagaggcagtggatctgggactgatttcactctcaccatcagcagtctgcaacctgaagattttgctacgtactactgtcaacagtgggatatgtttcctttgtcgttcggccaagggaccaaggtggaaatcaaacgg

SEQ ID NO: 604 - DNA, 324 bp, Homo sapiens
gacatccagatgacccagtctccatcctccctgtctgcatctgtaggagaccgtgtcaccatcacttgccgggcaagtcagaatattgattctcgtttaagttggtaccagcagaaaccagggaaagcccctaagctcctgatctataggacgtccgttttgcaaagtggggtcccatcacgtttcagtggtagtggatctgggacagatttcactctcaccatcagcagtctgcaacctgaagattttgctacgtactactgtcaacagtgggatatgtttcctttgatgttcggccaagggaccaaggtggaaatcaaacgg

SEQ ID NO: 605 - DNA, 324 bp, Homo sapiens
gacatccagatgacccagtctccatcctccctgtctgcatctgtaggagaccgtgtcaccatcacttgccgggcaagtcagaatattgattctcgtttaagttggtaccagcagaaaccagggaaagcccctaagctcctgatctatagggcgtccgttttgcaaagtggggtcccatcacgtttcagtggcagtggatctgggacagatttcactctcaccatcagcagtctgcaacctgaagattttgctacgtactactgtcaacagtgggatatgtttcctttgtcgttcggccatgggaccaaggtggaaatcaaacgg

SEQ ID NO: 606 - DNA, 324 bp, Homo sapiens
gacatccagatgacccagtctccatcctccctgtctgcatctgtaggagaccgtgtcaccatcacttgccgggcaagtcagaatattgattctcgtttaagttggtaccagcagaaaccagggaaagcccctaagctcctgatctataggacgtccgttttgcaaagtggggtcccatcacgtttcagtggcagtggatctgggacagatttcactctcaccatcagcagtctgcaacctgaagattttgctacgtactactgtcaacagtgggatatgtttcctttgatgttcggccaagggaccaaggtggaaatcaaacgg

SEQ ID NO: 607 - DNA, 324 bp, Homo sapiens
gacatccagatgacccagtctccatcctccctgtctgcatctgtaggagaccgtgtcaccatcacttgccgggcaagtcagaatattgattctcgtttaagttggtaccagcagaaaccagggaaagcccctaagctcctgatctatagggcgtccgttttgcaaagtggggtcccatcacgtttcagtggcactggatctgggacagatttcactctcaccatcagcagtctgcaacctgaagattttgctacgtactactgtcaacagtgggatatgtttcctttgtcgttcggccaagggaccaaggtggaaatcaaacgc

SEQ ID NO: 608 - DNA, 324 bp, Homo sapiens
gacatccagacgacccagtctccatcctccctgtctgcatctgtaggagaccgtgtcaccatcacttgccgggcaagtcagaatattgattctcgtttaagttggtaccagcagaaaccagggaaagcccctaagctcctgatctataggtcgtccgttttgcaaagtggggtcccatcacgtttcagtggcagtggatctgggacagatttcactctcaccatcagcagtctgcaacctgaagattttgctacgtattactgtcaacagtgggatatgtttcctttgatgttcggccaagggaccaaggtggaaatcaaacgg

SEQ ID NO: 609 - DNA, 324 bp, Homo sapiens
gacatccagatgacccagtctccatcctccctgtctgcatctgtaggagaccgtgtcaccatcacttgccgggcaagtcagaatattgattctcgtttaagttggtaccagcagaaaccagggaaagcccctaagctcctgatctatagggcgtccgttttgcaaagtggggtcccatcacgtttcagtggcagtggatctgggacagatttcactcttaccatcagcagtctgcaacctgaagattttgctacgtactactgccaacagtgggatatgtttcctttggcgttcggccaagggaccagggtggaaatcaaacgg

SEQ ID NO: 610 - DNA, 324 bp, Homo sapiens
gacatccagatgacccagtctccatcctccctgtctgcatctgtaggagaccgtgtcaccatcacttgccgggcaagtcagaatattgattctcgtttaagttggtaccagcagaaaccagggaaagcccctaagctcctgatctatagggcgtccgttttgcaaagtggggtcccatcacgtttcagtggcagtggatctgggacagatttcactctcaccatcagcagtctgcaacctgaagattttgctacgtaccactgtcaacagtgggatatgtttcctttgacgttcggccaagggaccaaggtggaaatcaaacgg

SEQ ID NO: 611 - DNA, 324 bp, Homo sapiens
gacatccagatgacccagtctccatcctccctgtctgcatctgtaggagaccgcgtcaccatcacttgccgggcaagtcagaatattgattctcgtttaagttggtaccagcagaaaccagggaaagcccctaagctcctgatctataggacgtccgttttgcagagcggggtcccatcacgtttcagtggcagtggatctgggacagttttcactctcaccatcagcagtctgcaacctgaagattttgctacgtactactgtcaacagtgggatatgtttcccctggcgttcggcaaagggaccaaggtggaaatcaaacgg

SEQ ID NO: 612 - DNA, 324 bp, Homo sapiens
gacatccaggtgacccagtctccatcctccctgtctgcatctgtaggagaccgtgtcaccatcacttgccgggcaagtcagaatattgattctcgtttaagttggtaccagcagaaaccagggaaagcccctaagctcctgatctatagggcgaccgttttgcaaagtggggtcccatcacgtttcagtggcagtgggtctgggacagatttcactctcaccatcagcagtctgcaacctgaagattttgctacgtactactgtcaacagtgggatatgtttcctttgacgttcggccaagggaccaaggtggaaatcaaacgg

SEQ ID NO: 613 - DNA, 324 bp, Homo sapiens
gacatccagatgacccagtctccatcctccctgtctgcatctgtaggagaccgtgtcaccatcacatgccgggcaagtcagaatattgattctcgtttaagttggtaccagcagaaaccagggaaagcccctaagctcctgatctataggacgtccgttttgcaaagtggggtcccaacacgtttcagtggcagtggatctgggacagatttcactctcaccatcagcagtctgcaacctgaagattttgctacgtactactgtcaacagtgggatatgtttcctttgacgttcggccaagggaccaaggtggaaatcaaacgg

SEQ ID NO: 614 - DNA, 324 bp, Homo sapiens
gacatccagatgacccagtctccatcctccctgtctgcatctgtaggagaccgtgtcaccatcacttgccgggcaagtcagaatattgattctcgtttaagttggtaccagcagaaaccagggaaagcccctaagctcctggtctatagggcgtccgttttgcaaagtggggtcccatcacgtttcagtggcagtggatctgggacagatttcactctcaccatcagcagtctgcaacctgaagattttgctacgtaccactgtcaacagtgggatatgtttcctttgacgctcggccaagggaccaaggtggaaatcaaacgc

SEQ ID NO: 615 - DNA, 324 bp, Homo sapiens
gacatccagatgacccagtctccatcctccctgtctgcatctgtaggagatcgtgtcaccatcacttgccgggcgagtcagaatattgattctcgtttaagttggtaccagcagaaaccagggaaagcccctaagctcctgatctatagggcgtccgttttgcaaagtggggtcccatcacgtttcagtggcagtggatctgggacagatttcactctcaccatcagcagtctgcaacctgaagattttgctacgtactactgtcaacagtgggatatgtttcctttggcgttcggccaagggaccaaggtggaaatcaaacgg

SEQ ID NO: 616 - DNA, 324 bp, Homo sapiens
gacatccagatgacccagtctccatcctccctgtctgcatctgtaggagaccgtgtcaccatcacttgccgggcaagtcagaatattgattctcgtttaagttggtaccaggagaaaccagggaaagcccctaagctcctgatctatagggcgtccgttttgcaaagtggggtctcatcacgtttcagtggcagtggatctgggacagatttcactctcaccatcagcagtctgcaacctgaagattttgctacgtactactgtcaacagtgggatatgtttcctttgacgttcggccgagggaccaaggtggaaatcaaacgg

SEQ ID NO: 617 - DNA, 324 bp, Homo sapiens
gacatccagatgacccagtctccatcctccctgtctgcatctgtaggagaccgtgtcaccatcacttgccgggcaagtcagaatattgattctcgtttaagttggtaccagcagaaaccagggaaagaccctaagctcctgatctataggtcgtccgttttgcaaagtggggtcccatcacgtttcagtggcagtggatctgggacagatttcactctcaccatcagcagtctgcaacctgaagattttgctacgtactactgtcaacagtgggatatgtttcctttgacgttcggccaagggaccaaggtggaaatcaaacgg

SEQ ID NO: 618 - DNA, 324 bp, Homo sapiens
gacatccagatgacccagtccccatcctccctgtctgcatctgtaggagaccgtgtcaccatcacttgccgggcaagtcagaatattgattctcgtttaagttggtaccagcagaaaccagggaaagcccctaagctcctgatctataggtcgtccattttgcaaagtggggtcccatcacgtttcagtggcagtggatctgggacagatttcactctcaccatcagcagtctgcaacctgaagattttgctacgtaccactgtcaacagtgggatatgtttcctttgacgttcggccaagggaccaaggtggaaatcaaacgg

SEQ ID NO: 619 - DNA, 324 bp, Homo sapiens
gacatccagatgactcagtctccatcctccctgtctgcatctgtaggagaccgtgtcaccatcacttgccgggcaagtcagaatattgattctcgtttaagttggtaccagcagaaaccagggaaagaccctaagctcctgatctatagggcgtccgttttgcaaagtggggtcccatcacgtttcagtggcagtgggtctgggacagatttcactctcaccatcagcagtctgcaacctgaagattttgctacgtactactgtcaacaatgggatatgtttcctttgacgttcagccaagggaccaaggtggaaatcaaacgg

SEQ ID NO: 620 - DNA, 324 bp, Homo sapiens
gacatccagatgacccagtctccatcctccctgtctgcatctgtaggagaccgtgtcaccatcacttgccgggcaagtcggaatattgattctcgtttaagttggtaccagcagaaaccagggaaagcccctaagctcctgatctataggacgtccgttttgcaaagtggggtcccatcacgtttcagtggcagtggatctgggacagatttcactctcaccatcagcagtctgcaacctgaagattttgctacgtactactgtcaacagtgggatatgtttcctttgatgttcggccaagggaccaaggtggaaatcaaacgg

SEQ ID NO: 621 - DNA, 324 bp, Homo sapiens
gacatccagatgacccagtctccatcctccctgtctgcatctgtaggagaccgtgtcaccatcacttgccgggcaagtcagaatattgattctcgtttaagttggtaccaggagaaaccagggaaagcccctaagctcctgatctataggacgtccgttttgcaaagtggggtcccatcacgtttcagtggcagtggatctgggacagatttcactctcaccatcagcagtctgcaacctgaagattttgccacgtactactgtcaacagtgggatatgtttcctctgacgttcggccaagggaccagggtggaaatcaaacgg

SEQ ID NO: 622 - DNA, 324 bp, Homo sapiens
gacatccagatgacccagtctccatcctccctgtctgcatctgtaggagaccgtgtcaccatcacttgccgggcaagtcagaatattgattctcgtttaagttggtaccagcagaaaccagggaaagcccctaagctcctgatctataggacgtccgttttgcaaagtggggtcccatcacgtttcagtggcagtggatctgggacagaattcactctcaccatcagcagtctgcaacctgaagattttgctacgtactactgtcaacggtgggatatgtttcctttgacgttcggccaagggaccaaggtggaaatcaaacgg

SEQ ID NO: 623 - DNA, 324 bp, Homo sapiens
gacatccagatgacccagtctccttcctccctgtctgcatctgtaggagaccgagtcaccatcacttgccgggcaagtcagaatattgattctcgtttaagttggtaccagcagaaaccagggaaagcccctaagctcctgatctataggacgtccgttttgcaaagtggggtcccatcacgtttcagtggcagtggatctgggacagatttcactctcaccatcagcagtctgcaacctgaagattttgctacgtactactgtcaacagtgggatatgtttcctttgacgttcggccatgggaccaaggtggaaatcaaacgg

SEQ ID NO: 624 - DNA, 324 bp, Homo sapiens
gacatccagatgacccagtctccatcctccctgtctgcatctgttggagaccgtgtaaccatcacttgccgggcaagtctgaagattgagaatgatttagcttggtaccagcagaaaccagggaaagcccctaagcttctgatctattatacttccattttgcaaagtggggtcccatcacgtttcagtggcagtggatctgggacagatttcactctcaccatcagcagtctgcaacctgaagattttgctacgtactactgtcaacagcggaggtatgcgcctgcgacgttcggccaagggaccaaggtggaaatcaaacgg

SEQ ID NO: 625 - DNA, 324 bp, Homo sapiens
gacatccagatgacccagtctccatcctccctgtctgcatctgtaggagaccgtgtaaccatcacttgccggacaagtcagaagattgagaatgatttagcttggtaccagcagaaaccagggaaagcccctaagctcctgatctattatacttccattttgcaaagtggggtcccatcacgtttcagtggcagtggatctgggacagatttcactctcaccatcagcagtctgcaacctgaagattttgctacgtactactgtcaacagcggaggtatgtgcctgcgacgttcggccaagggaccaaggtggaaatcaaacgg

SEQ ID NO: 626 - DNA, 324 bp, Homo sapiens
gacatccagatgacccagtctccatcatccctgtctgcatctgtaggagaccgtgtaaccatcacttgccgggcgagtcagaagattgagaatgatttagcttggtaccagcagaaaccagggaaagcccctaagctcctgatctattatacttccattttgcaaagtggggtcccatcacgtttcagtggcagtggatctgggacagatttcactctcaccatcagcagtctgcaacctgaagattttgctacgtactactgtcaacagcggaggtatgcgcctgcgacgttcggccaagggaccaaggtggaaatcaaacgg

SEQ ID NO: 627 - DNA, 324 bp, Homo sapiens
gacatccagttgacccagtctccatcctccctgtctgcatctgtaggagaccgtgtaaccatcacttgccgggcaagtcagaagattgagaatgatttagcttggtaccagcagaaaccagggaaagcccctaagctcctgatctattatacttccattttgcaaagaggggtcccatcacgtttcagtggcagtggatctgggacagatttcactctcaccatcagcagtctgcaacctgaagatattgctacgtactactgtcaacagcggaggtatgtgcctgcgacgttcggccaagggaccaaggtggaaatcaaacgg

SEQ ID NO: 628 - DNA, 324 bp, Homo sapiens
gacatccagatgacccagtctccatcctccctgtctgcatctgtaggagaccgtgtaaccatcacttgccgggcaagtcagaagattgagaatgatttagcttggtaccagcagaaaccagggaaagcccctaagctcctgatctattatacttccattttgcaaagtggggtcccatcacgtttcattggcagtggatctgggacagatttcactctaaccatcagcagtctgcaacctgaagattttgctacgtactactgtcaacagcggaggtatgtgcctgcgacgttcggcccagggaccaaggtggaaatcaaacgg

SEQ ID NO: 629 - DNA, 324 bp, Homo sapiens
gacatccagatgacccagtctccatcctccctgtctgcatctgtaggagaccgtgtaaccatcacttgccgggcaagtcagaagattgagaatgatttagcttggtaccagcagaaaccaggtaatgcccctaagctcctgatctattatacttccattttgcatagtggggtcccatcacgtttcagtggcagtggatctgggacagatttcactctcaccatcagcagtctgcaacctgaagattttgctacgtactactgtcaacagcggaggtatgtgcctgcgacgttcggccaagggaccaaggtggaaatcaaacgg

SEQ ID NO: 630 - DNA, 324 bp, Homo sapiens
gacatccagatgacccagtctccgtcctccctgtctgcatctgtaggagaccgtgtaaccatcacttgccggacaagtcagaagattgagaatgatttagcttggtaccagcagagaccagggaaagcccctaagctcctgatctattatacttccattttgcaaagtggggtcccatcacgtttcagtggcagtggatctgggacagatttcactctcaccatcagcagtctgcaacctgaagattttgctacgtactactgtcaacagcggaggtatgtgcctgcgacgttcggccaagggaccaaggtggaaatcaaacgg

SEQ ID NO: 631 - DNA, 324 bp, Homo sapiens
gacatccagatgacccagtctccatcctccctgtctgcatccgttggagaccgtgtaaccatcacttgccgggcaagtcagaagattgagaatgatttagcttggtaccagcagaaaccagggaaagcccctaagctcctgatctattatacttccattttgcaaagtggggtcccatcacgtttcagtggcagtggatctgggacagatttcactctcaccatcagcagtctgcaacctgaagattttgctacgtactactgtcaacagcggaggtatgcgcctgcgacgttcggccaagggaccaaggtggaaatcaaacgg

SEQ ID NO: 632 - DNA, 324 bp, Homo sapiens
gacatccagatgacccagtctccatcctccctgtctgcatctgtaggagaccgtgtaaccatcacttgccgggcaagtcagaagattgagaatgatttagcttggtaccagcagaaaccagggaaagcccctaagctcctgatctattatacttccattttgcaaagtgggatcccatcacgtttcagtggcagtggatctgggacagatttcactctcaccatcagcagtctgcaacctgaagattttgctacgtactactgtcaacagcggaggtatgcgcctgcgacgttcggccaagggaccaaggtggaaatcaaacgg

SEQ ID NO: 633 - DNA, 324 bp, Homo sapiens
gacatccagatgacccagtctccatcctccctgtctgcatctgtaggagaccgtgtaaccatcacttgccgggcaagtcagaagattgagaatgatttagcttggtaccagcagaaaccagggaaagcccctaagctcctgatctattatacttccattttgcaaagtggggtcccatcacgtttcagtggcagtggatctgggacagatttcactctcaccatcagcagtctgcaacctgaagattttgctacgtactactgtcaacagcggaggtatgtgcctgcgtcgttcggccaagggaccaaggtggaaatcaaacgg

SEQ ID NO: 634 - DNA, 324 bp, Homo sapiens
gacatccagatgacccagtctccatcctccctgtctgcatctgtaggagaccgtgtaaccatcacttgccgggcaagtcagaagattgagaatgatttagcttggtaccagcagaaaccagggaaagcccctaagctcctgatctattatacttccattttgcaaagaggggtcccatcacgtttcagtggtagtggatctgggacagatttcactctcaccatcagcagtctgcaacctgaagattttgctacgtactactgtcaacagcggaggtatgcgcctgcgacgttcggccaagggaccaaggtggaaatcaaacgg

SEQ ID NO: 635 - DNA, 324 bp, Homo sapiens
gacatccagatgacccagtctccatcctccctgtctgcatctgtaggagatcgtgtaaccatcacttgccggacaagtcagaagattgagaatgatttagcttggtaccagcagaaaccagggaaagcccctaagctcctgatctattatacttccattttgcaaagtggggtcccatcacgtttcagtggcagtggatctgggacggatttcactctcaccatcagcagtctgcaacctgaagattttgctacgtactactgtcaacagcggaggtatgcgcctgcgacgttcggccaagggaccaaggtggaaatcaaacgg

SEQ ID NO: 636 - DNA, 324 bp, Homo sapiens
gacatccagatgacccagtctccatcctccctgtctgcatctgtaggagaccgtgtaaccatcacttgccgggcaaatcagaagattgagaatgatttagcttggtaccagcagaaaccagggaaagcccctaagctcctgatctattatacttccattttgcaaagtggggtcccatcacgtttcagaggcagtggatctgggacagatttcactctcaccatcagcagtctgcaacctgaagattttgctacgtactactgtcaacagcggaggtatgtgcctgcgacgttcggccaagggaccaaggtggaaatcaaacgg

SEQ ID NO: 637 - DNA, 324 bp, Homo sapiens
gacatccagatgacccagtctccatcctccctgtctgcatctgtaggagaccgtgtaaccatcacttgccgggcaagtcagaagatagagaatgatttagcttggtaccagcagaaaccagggaaagcccctaagctcctgatctattatacttccattttgcatagtggggtcccatcacgtttcagtggcagtggatctgggacagatttcactctcaccatcagcagtctgcaacctgaagattttgctacgtactactgtcaacagcggaggtatgtgcctgcgacgttcggccaagggaccaaggtggaaatcaaacgg

SEQ ID NO: 638 - DNA, 324 bp, Homo sapiens
gacatccagatgacccagtctccatcctccctgtctgcatctgtaggagaccgtgtaaccatcacttgccaggcaagtaagaagattgagaatgatttagcttggtaccagcagaaaccagggaaagcccctaagctcctgatctattatacttccattttgcaaagtggggtcccatcacgtttcagtggcagtggatctgggacagatttcactctcaccatcagcagtctgcaacctgaagattttgctacgtactactgtcaacagcggaggtatgtgcctgcgacgttcggccaagggaccaaggtggaaatcaaacgg

SEQ ID NO: 639 - DNA, 324 bp, Homo sapiens
gacatccagatgacccaggctccatcctccctgtccgcatctgtaggagaccgtgtaaccatcacttgccggtcaagtcagaagattgagaatgatttagcttggtaccagcagaaaccagggaaagcccctaagctcctgatctattatacttccattttgcaaagtggggtcccatcacgtttcagtggcagtggatctgggacagatttcactctcaccatcagcagtctgcaacctgaagattttgctacgtactactgtcaacagcggaggtatgtgcctgcgacgttcggccaagggaccaaggtggaaatcaaacgg

SEQ ID NO: 640 - DNA, 324 bp, Homo sapiens
gacatccagatgacccagtctccatcctccctgtctgcatctgtaggagaccgtgtcaccatcacttgccgggcaagtcaggatattggtgataggttacggtggtatcagcagaatccagggaaagcccctaagctcctgatctatcatgggtccaggttggaaagtggggtcccatcacgtttcagtggcaatagatctgggacagatttcactcttaccatcagcagtctgcaacctgaagattttgctacgtactactgtcaacagcagtggtttcgtccttatacgttcggccaagggaccaaggtggaaatcaaacgg

SEQ ID NO: 641 - DNA, 324 bp, Homo sapiens
gacatccagatgacccagtctccatcctccctgtctgcatctgtaggagaccgtgtcaccatcacttgccgggcaagtcaggatattggtggtaggttacggtggtatcagcagaaaccagggaaagcccctaagctcctgatctatcatgggtccaggttggatagtggggtcccatcacgtttcagtggcagtagatctggggcagatttcactcttaccatcagcagtctgcaacctgaagattttgctacgtactactgtcaacagcagtggtatcgtccttatacgttcggccaagggaccaaggtggaaatcaaacgg

SEQ ID NO: 642 - DNA, 324 bp, Homo sapiens
gacatccagatgacccagtccccatcctccctgtctgcatctgtaggagaccgtgtcaccatcacttgccgggcaagtcaggatattggtgataggttacggtggtatcagcagaaaccagggaaagcccctaagctcctgatctatcatgggtccaggttggatagtggggtcccatcacgtttcagtggcagtagatctgggacagattacactcttaccatcagcagtctgcaacctgaagattttgctacgtactactgtcaacagcagtggtttcgtccttatacgttcggccaagggaccaaggtggaaatcaaacgg

SEQ ID NO: 643 - DNA, 324 bp, Homo sapiens
gacatccagatgacccagtctccatcctccctgtctgcatctgtaggagaccgtgtcaccatcacttgccgggcaagtcaggatattggtgataggttacggtggtatcagcagaaaccagggaaagcccctaagctcctgatctatcatgggtccaggttagaaagtggggtcccatcacgtttcagtggcagtagatctgggacagatttcactcttaccatcagcagtctgcaacctgaagattttgctacgtactactgtcaacagcagtggcttcgtccttatacgttcggccaagggaccagggtggaaatcaaacgg

SEQ ID NO: 644 - DNA, 324 bp, Homo sapiens
gacatccagatgacccagtctccatcctccctgtctgcatctgtaggagaccgtgtcaccatcacttgcggggcaagtcaggatattggtgataggttacggtggtatcagcagaaaccagggaaagcccctaagctcctgatctatcatgggtccaggttggaaagtggggtcccatcacgtttcagtggcagtagatctgggacagatttcactcttaccatcagcagtctgcaacctgaagattttgctacgtactactgtcaacagcagtggtttcgtccttatacgttcggccaagggaccaaggtggaaatcaaacgg

SEQ ID NO: 645 - DNA, 324 bp, Homo sapiens
gacatccagatgacccagtctccatcctccctgtctgcatctgtaggagaccgtgtcaccatcacttgccgggcaagtcaggatattggtgataggttacggtggtatcagcagaaaccagggagagcccctaagctcctgatctatcatgggtccaggttggaaagtggggtcccatcacgtttcagtggcagtagatctgtgacagatttcactcttaccatcagcagtctgcaacctgaagattttgctacgtactactgtcaacagcagtggtttcgtccttatacgttcggccaagggaccaaggtggaaatcaaacgg

SEQ ID NO: 646 - DNA, 324 bp, Homo sapiens
gacatccagatgacccagtctccatcctccctatctgcatctgtaggagaccgtgtcaccatcacttgccgggcaagtcaggttattggtgataggttacggtggtatcagcagaaaccagggaaagcccctaagctcctgatctatcatgggtccaggttggagagtggggtcccatcacgtttcagtggcagtagatctgggacagatttcactcttaccatcagcagtctgcaacctgaagattttgctacgtactactgtcaacagcagtggtttcgtccttatacgtttggccaagggaccaaggtggaaatcaaacgg

SEQ ID NO: 647 - DNA, 324 bp, Homo sapiens
gacatccagatgacccagtctccatcctccctgtctgcatctgtaggagaccgtgtcaccatcacttgccgggcaagtcaggatattggtgataggttacggtggtatcagcagaaaccagggaaagcccctaagctcctgatctatcatgggtccaggttggaaagtggggtcccatcacgtttcagtggcaatagatctgggacagttttcactcttaccatcagcagtctgcaacctgaagattttgctacgtactactgtcaacagcagtggtttcgtccttatacgttcggtcaagggaccaaggtggaaatcaaacgg

SEQ ID NO: 648 - DNA, 324 bp, Homo sapiens
gacatccagatgacccagtctccatcctccctgtctgcatctgtaggagaccgtgtcaccatcacttgccgggcaagtcaggatattggtgataggttacgctggtatcagcagaaaccagggaaagcccctaagctcctgatctatcatgggtccaggttggaaagtggggtcccatcacgtttcagaggcagtagatctgggacagatttcactcttaccatcagcagtctgcaacctgaagattttgctacgtactactgtcagcagcagtggtttcgtccttatacgttcggccaagggaccaaggtggaaatcaaacgg

SEQ ID NO: 649 - DNA, 324 bp, Homo sapiens
gacatccagatgacccagtctccatcctcgctgtctgcatctgtaggagaccgtgtcaccatcacttgccgggcaagtcaggatattggtgataggttacggtggtatcagcagaaaccagggaaagcccccaagctcctgatctatcatgggtccaggttggaaagtggggtcccgtcacgtttcagtggcagtagatatgggacaaatttcactcttaccatcagcagtctgcaacctgaagattttgctacgtactactgtcaacagcagtggtttcgtccttatacgttcggccaagggaccaaggtggaaatcaaacgg

SEQ ID NO: 650 - DNA, 324 bp, Homo sapiens
gacatccagatgacccagtctccatcctccctgtctgcatctgtaggagaccgtgtcaccatcacttgccgggcaagtcaggatattggtgataggttacggtggtatcagcagaaaccagggaaagcccctaagctcctgatctatcatgggtccaggttggaaagtggggtcccatcacgtttcagtggcagtagatttgggacagatttcactcttaccatcagcagtctgcaacctgatgattttgctacgtactactgtcaacagcagaggtttcgtccttatacgttcggccaagggaccaaggtggaaatcaaacgg

SEQ ID NO: 651 - DNA, 324 bp, Homo sapiens
gacatccagatgacccagtctccatcctccctgtctgcatctgtaggagaccgtgtcaccatcacctgccgggcaagtcaggatattggtgataggttacggtggtatcagcagaaaccagggaaagcccctaagctcatgatctatcatgggtccaggttggaaagtggggtcccatcacgtttcagtggcagtagatctgggacagatttcactcttaccatcagcagtctgcaacctgaagattttgctacgtactactgtcaacagcagtggtttcgtccttatacgttcggccaagggaccaaggtggaaatcaaacgg

SEQ ID NO: 652 - DNA, 324 bp, Homo sapiens
gacatccagatgacccagtctccatcctccctgtctgcttctgtaggagaccgtgtcaccatcacttgccgggcaagtcaggatattggtgataggttacggtggtatcagcagaaaccagggaaagcccctaagctcctgatctatcatgggtccaggttggaaagtggggtcccatcacgtttcagtggcggtagatctgggacagatttcactcttaccattagtagtctgcaacttgaagattttgctacgtactactgtcaacagcagtggtttcgtccttatacgttcggccaagggaccaaggtggaaatcaaacgg

SEQ ID NO: 653 - DNA, 324 bp, Homo sapiens
gacatccagatgacccagtctccatcctccctgtctgcatctgtaggagaccgtgttaccatcacttgccgggcaagtcgggatattggtgataggttacggtggtatcagcagaaaccagggaaagcccctaagctcctgatctatcatgggtccaggttggaaagtggggtcccatcacgtttcagtggcagtagatctgggacagatttcactcttaccatcagcagtctgcaacctgaagattttgctacgtactactgtcaacagcagtggtttcgtccttatacgtttggccaagggaccaaggtggaaatcaaacgg

SEQ ID NO: 654 - DNA, 324 bp, Homo sapiens
gacatccagatgacccagtctccatcctccctgtctgcatctgtaggagaccgtgtcaccatcacttgccgggcaagtcaggatattggttataggttacggtggtatcagcagaaaccagggaaagcccctatgctcctgatctatcatgggtccaggttggaaagtggggtcccatcacgtttcagtggcagaagatctgggacagatttcactcttaccatcagcagtctgcaacctgaagattttgctacgtactactgtcaacagcagtggtttcgtccttatacgttcggacaagggaccaaggtggaaatcaaacgg

SEQ ID NO: 655 - DNA, 324 bp, Homo sapiens
gacatccagatgacccagtctccatcctccctgtcagcatctgtaggagaccgtgtcaccatcacttgccgggcaagtcaggatattggtgataggttacggtggtatcagcagaaaccagggaaagcccctaagctcctgatctatcatgggtccaggttggaaagtggggtcccatcacgtttcagcggcagtagatctgggacagatttcactcttaccatcagcagtctgctacctgaagattttgctacgtactactgtcaacagcagaggtttcgtccttatacgttcggccaagggaccaaggtggaaatcaaacgg

SEQ ID NO: 656 - DNA, 324 bp, Homo sapiens
gacatccagatgacccagtctccgtcctccctgtctgcatctgtaggagaccgtgtctccatcacttgtcgggcaagtcaggatattggtgataggttacggtggtatcagcagaaaccagggaaagcccctaagctcctgatctatcatgggtccaggttggaaagtggggtcccatcacgtttcagaggcagcagatctgggactgatttcaatcttaccatcagcagtctgcaacctggagattttgctacgtactactgtcaacagcagtggtttcgtccttatacgttcggccaagggaccaaggtggaaatcaaacgg

SEQ ID NO: 657 - DNA, 324 bp, Homo sapiens
gacatccagatgacccagtctccatcctccctgtctgcatctgtaggagaccgtgtcaccatcacttgccgggcaagtcaggatattggtgataggttacggtggtatcagcagaaaccagggaaagcccctaagctcctgatctatcatgggtccaggttggaaagtagggtcccatcacgattcagtggcaatagatctgggacagatttcactcttagcatcagcagtctgcaacctgaagattttgctacgtactactgtcaacagcagcggtttcgtccttatacgttcggccaagggaccaaggtggaaatcaaacgg

SEQ ID NO: 658 - DNA, 324 bp, Homo sapiens
gacatccagatgacccagtctccatcctccctgtctgcatctgtaggagaccgtgtcaccatcacttgccgggcaagtcaggatattggtgataggttacggtggtatcagcagaaaccagggaaagcccctaagctcatgatctatcatgggtccaggttggaaagtggggtcccatcacgtttcagtggcagtagatctgggacagatttcactcttaccatcagcagtctgcaacctgaagattttgctacgtactactgtcaacagcagtggtctcgtccttatacgttcggccaagggaccaaggtggaaatcaaacgg

SEQ ID NO: 659 - DNA, 324 bp, Homo sapiens
gacatccagatgacccagtctccatcctccctgtctgcatctgtaggagaccgtgtcgccatcacttgccgggctagtcaggatattggtgacaggttacggtggtatcatcagaaaccagggaaagcccctaagctcctgatctatcatgggtccaggttggaaagtggggtcccatcacgtttcagtggcagtagatctgggacagatttcactcttaccatcagcagtctgcaacctgaagattttgctacgtactactgtcaacagcagtggtttcgtccttatacgttcggccaagggaccaaggtggaaatcaaacgg

SEQ ID NO: 660 - DNA, 324 bp, Homo sapiens
gacatccagatgacccagtctccatcctccctgtctgcatctgtaggagaccgtatcaccatcacttgccgggcaagtcaggatattggtgataggttacggtggtatcagcagagaccagggaaagcccctaagctcctggtctatcatgggtccaggttggaaagtggggtcccatcacgtttcagtggcagtagatctgggacagatttcactcttaccatcagcagtctgcaacctgaagattatgctacgtactactgtcagcagcagtggtttcgtccttatacgttcggccaagggaccaaggtggaaatcaaacgg

SEQ ID NO: 661 - DNA, 324 bp, Homo sapiens
gacatccagatgacccagtctccatcctccctgtctgcatctgtaggagaccgtgtcaccatcacttgccgggcaagtcaggatattggtgataggttacggtggtatcagcagaaaccagggaaagcccctaagctcctgatctatcatgggtccaggttggaaagtggggtcccatcacgattcagtggcggtagatctgggacagatttcactcttaccatcagcagtctgcaacctgaagattctgctacgtactactgtcaacagcagtggcttcgtccatatacgttcggccaagggaccaaggtggaaatcaaacgg

SEQ ID NO: 662 - DNA, 324 bp, Homo sapiens
gacatccagatgactcagtctccatcctccctgtcagcatctgtaggagaccgtgtcaccatcacttgccgggcaagtcaggatattggtgataggttacggtggtatcagcagaaaccagggaaagcccctaagctcatgatctatcatgggtccaggttggaaagtggggtcccatcacgtttcagcggcagtagatctgggacagatttcactcttaccatcagcagtctgcaacctgaagattttgctacgtactactgtcaacagcagtggtttcgtccttatacgttcggccaagggaccaaggtggaaatcaaacgg

SEQ ID NO: 663 - DNA, 324 bp, Homo sapiens
gacatccagatgacccagtctccatcctccctgtctgcatctgtaggagaccgcgtcaccatcacttgccgggcaagtcaggatattggtgataggttacggtggtatcagcagaaaccagggaaagcccctaagctcatgatctatcatgggtccaggttggaaagtggggtcccaccacgtttcagtggcagtaggtctgggacagatttcactcttaacatcagcagtctgcaacccgatgattttgctacgtactactgtcaacagcagtggtttcgtccttatacgttcggccaagggaccaaggtggaaatcaaacgg

SEQ ID NO: 664 - DNA, 324 bp, Homo sapiens
gacatccagatgacccagtctccatcctccctgtctgcatctgtaggagaccgtgtcaccatcacttgccgggcaagtcaggatattggtgataggttacggtggtatcagcagaaaccagggaaagcccctaggctcctgatctatcatgggtccaggttggaaagtggggtcccatcacgtttcagtggcagtagatctgggacagatttcactcttaccttcagcagtctgcaacctgaagattttgctacgtactactgtcaacagcagtggatacgtccttatacgttcggccaagggaccaaggtggaaatcaaacgg

SEQ ID NO: 665 - DNA, 324 bp, Homo sapiens
gacatccagatgacccagtctccatcctccctgtctgcatctgtaggagaccgtgtcaccatcacttgccgggcaagtcgggatattggtgataggttacggtggtatcagcagaaaccagggaaagcccctaagctcctgatctatcatgggtccaggttggaaagtggggtcccatcacgtttcagtggcagtagatctgggacagatttcactctcaccatcagcagtctgcaacctgaagattttgctacgtactactgtcaacagcagtggtttcgtccttatacgttcggccaagggaccaaggtggaaatcaaacgg

SEQ ID NO: 666 - DNA, 324 bp, Homo sapiens
gacatccagatgacccagtctccaacctccctgtctgcatctgtaggagaccgtgttaccatcacttgccgggcaagtcaggatattggtgataggttacggtggtatcagcagagaccagggaaagcccctaagctcctgatctatcatgggaccaggttggaaagtggggtcccatcacgattcagtggcagtagatctgggacagatttcactcttaccatcagcagtctgcaacctgaagattttgctacgtactactgtcaacagcagtggtttcgtccttatacgttcggccaagggaccaaggtggaaatcaaacgg

SEQ ID NO: 667 - DNA, 324 bp, Homo sapiens
gacatccagatgacccagtctccatcctccctgtctgcatctgtaggagaccgtgtcaccatcacttgccgggcaagtcaggatattggtgataggttacggtggtatcagcagaaaccagggaaagcccctaagctcctgatctatcatgggtccaggttggaaagtgggatcccatcacgtttcagtggcagtagatctgggacagatttcactcttaccatcagcagtctacaacctgaagattttgctacgtactactgtcaacagcagtggtttcgcccttatacgttcggccaagggaccaaggtggaaatcaaacgg

SEQ ID NO: 668 - DNA, 324 bp, Homo sapiens
gacatccagatgacccagtctccatcctccctgtctgcatccgtaggagaccgtgtcaccatcacttgccgggcaagtcaggatattgatgataggttacggtggtatcagcagaaaccagggaaagcccctaagctcctgatctatcatgggtccaggttggatagtggggtcccatcacgtttcagtggtagtagatctgggacagatttcactcttaccatcagcagtctgcaacctgaagattttgctacgtactactgtcaacagcagtggtttcgtccttatacgttcggccaagggaccaaggtggaaatcaaacgg

SEQ ID NO: 669 - DNA, 324 bp, Homo sapiens
gacatccagatgacccagtctccatcctccctgtctgcatctgtaggagaccgtgtcaccatcacttgccgggcaagtcaggatattggtgataggttacgatggtatcagcagaaaccagggaaagcccctaagctcctgatctatcatgggtccagattggacagtggggtcccatcacgtctcagtggcagtagatctgggacagatttcactcttaccatcagcagtctgcaacctgaagattttgctacgtactactgtcaacagcagtggtttcgtccttatacgttcggccaagggaccaaggtggaaatcaaacgg

SEQ ID NO: 670 - DNA, 324 bp, Homo sapiens
gacatccagatgacccagtctccatcctccctgtctgcatctgtaggagaccgtgtcaccatcacttgccgggcaagtcaggatattggtgataggttacggtggtatcagcagaaaccagggaaagcccctaagctcctgatctatcatgggtccaggttggaaagtggggtcccatcacgtttcagtggcggtagatctgggacagatttcactcttaccatcagaagtctgcaaccagaagattttgctacgtactactgtcaacagcagtggtttcgtccttatacgttcggccaagggaccaaggtggaaatcaaacgg

SEQ ID NO: 671 - DNA, 324 bp, Homo sapiens
gacatccagatgacccagtctccatcctccctgtctgcatctgtaggagaccgtgtcaccatcacttgccgggcaagtcgggatattggtgataggttacggtggtatcaacagaaaccagggaaagcccctaagctcctgatctatcatgggtccaggttggaaagtggggtcccatcacgtttcagtggcagtagatctgggacagatttcactcttaccatcagcagactgcaacctgaagattttgctacgtactactgtcaacagcagtggtttcgtccttatacgttcggccaagggaccaaggtggaaatcaaacgg

SEQ ID NO: 672 - DNA, 324 bp, Homo sapiens
gacatccagatgacccagtctccatcctccctgtctgcatctgtaggagaccgtgtcaccatcacttgccgggcaagtcaggatattggtgataggttacggtggtatcagcagaaaccagggaaagcccctaagctcctgatctatcatgggtccaggttggaaagtggggtgccatcacgtttcagtggcggtagatctgggacagatttcactcttaccatcagcagtctgcaacctgaagattttgctacgtactactgtcaacagcagcggtttcgtccttatacgttcggccaggggaccaaggtggaaatcaaacgg

SEQ ID NO: 673 - DNA, 324 bp, Homo sapiens
gacatccagatgacccagtctccatcctccctgtctgcaactgtaggagaccgtgtcaccatcacttgccgggcaagtcaggatattggtgataggttacggtggtatcagcagaaaacagggaaagcccctaagctcctgatctatcatgggtccaggttggaaagtggggtcccatcacgtttcagtggcagtagatctgggacagatttcactcttaccatcagcagtctgcaacctgaagattttgctacgtactactgtcaacagcagtggtttcgtccttatacgttcggccaagggaccaaggtggaaatcaaacgg

SEQ ID NO: 674 - DNA, 324 bp, Homo sapiens
gacatccagatgacccagtctccatcccgcctgtctgcgactgtaggagaccgtgtcaccatcacttgccgggcaagtcaggatattagtgataggttacgatggtatcagcagaaacccgggaaagcccctaagctcctgatctatcatgggtccaggttggaaagtggggtcccatcacgtttcagtggcagtagatctgggacagatttcgctcttaccatcagcagtctgcaacctgaagattttgctacgtactactgtcaacagcagtggtttcgtccatatacgttcggccaagggaccaaggtggaaatcaaacgg

SEQ ID NO: 675 - DNA, 324 bp, Homo sapiens
gacatccagatgacccagtctccatcctccctgtctgcatctgtgggagaccgtgtcaccatcacttgccgggcaagtcaggatatcggtgataggttacggtggtatcagcagaaaccagggaaagcccctaagctcctgatctatcatgggtccaggttggaaagtggggtcccttcacgtttcagtggcagtagatctgggacagatttcgctcttaccatcagcagtctgcaacctgaagattttgctacgtactactgtcaacagcagtggtttcgaccttatacgttcggcccagggaccaaggtggaaatcaaacgg

SEQ ID NO: 676 - DNA, 324 bp, Homo sapiens
gacatccagatgacccagtctccatcctccctgtctgcatctgtaggagaccgtgtcaccatcacttgccgggcaagtcgggatataggtgataggttacggtggtatcagcagaaaccagggaaagcccctaagctcctgatctatcatgggtccaggttggaaagtggggtcccatcacgtttcagtggcagtagatctgggacagatttcactcttaccatcagcagtctgcaacctgaagattttgctacgtactactgccaacagcagtggtttcgtccttatacgttcggccaagggaccaaggtggaaatcaaacgg

SEQ ID NO: 677 - protein, 244 aa, Homo sapiens
DIQMTQSPSSLSASVGDRVTITCRASQNIDSRLSWYQQKPGKAPKLLIYRTSVLQSGVPSRFSGSGSGTDFTLTISSLQPEDFATYYCQQWDMFPLTFGQGTKVEIKRSSGGGGSGGGGSGGGGSGGGGSGGGGSTDIQMTQSPSSLSASVGDRVTITCRASQDIGDRLRWYQQKPGKAPKLLIYHGSRLESGVPSRFSGSRSGTDFTLTISSLQPEDFATYYCQQQWFRPYTFGQGTKVEIKR

SEQ ID NO: 678 - DNA, 732 bp, Homo sapiens
gacatccagatgacccagtctccatcctccctgtctgcatctgtaggagaccgtgtcaccatcacttgccgggcaagtcagaatattgattctcgtttaagttggtaccagcagaaaccagggaaagcccctaagctcctgatctataggacgtccgttttgcaaagtggggtcccatcacgtttcagtggcagtggatctgggacagatttcactctcaccatcagcagtctgcaacctgaagattttgctacgtactactgtcaacagtgggatatgtttcctttgacgttcggccaagggaccaaggtggaaatcaaacgctcgagcggtggaggcggttcaggcggaggtggcagcggcggtggcgggtcaggtggtggcggaagcggcggtggcgggtcgacggacatccagatgacccagtctccatcctccctgtctgcatctgtaggagaccgtgtcaccatcacttgccgggcaagtcaggatattggtgataggttacggtggtatcagcagaaaccagggaaagcccctaagctcctgatctatcatgggtccaggttggaaagtggggtcccatcacgtttcagtggcagtagatctgggacagatttcactcttaccatcagcagtctgcaacctgaagattttgctacgtactactgtcaacagcagtggtttcgtccttatacgttcggccaagggaccaaggtggaaatcaaacgg

SEQ ID NO: 679 - protein, 108 aa, Homo sapiens; identical to SEQ ID NO: 540

SEQ ID NO: 680 - DNA, 324 bp, Homo sapiens; identical to SEQ ID NO: 178

SEQ ID NO: 681 - DNA, 323 bp, Homo sapiens
gacatccagatgacccagtctccatcctccctgtctgcatctgtaggagaccgtgtcaccatcacttgccgggcaagtcagagtattggtccgtggttaagttggtaccagcagaaaccagggaaagcccctaagctcctgttctatcaggtttcccgtctgcaaagtggggtcccatcacgtttcagtggcagtggatctgggacagatttcactctcaccatcatcagtctgcaacctgaagattttgctacgtactactgtcaacagaatcttgcgcctccttatacgttcggccaagggaccaaggtggaaatcaaacg

SEQ ID NO: 682 - DNA, 324 bp, Homo sapiens
gacatccagatgacccagtctccatcctccctgtctgcatctgtaggagaccgtgtcaccatcacttgccgggcaagtcagagtattggtccgtggttaagttggtatcagcagaaaccagggaaagcccctaagctcctgttctatcaggtttcccgtctgccaagtggggtcccatcacgtttcagtggcagtggatctgggacagatttcactgtcaccatcagcagtctgcaacctgaagattttgctacgtactactgtcaacagaatcttgcgcctccttatacgttcggccaagggaccaaggtggaaatcaaacgg

SEQ ID NO: 683 - DNA, 324 bp, Homo sapiens
gacatccagatgacccagtctccatcctccctgtcagcatctgtaggagaccgtgtcaccatcacttgccgggcaagtcagagtattggtccgtggttaagttggtatcagcagaaaccagggaaagcccctaagctcctgttctatcaggtttcccgtctgcgaagtggggtcccatcacgtttcagtggcagtggatctgggacagatttcactctcaccatcagcagtctgcaacctgaagattttgcttcgtactactgtcaacagaatcttgcgcctccttatacgttcggccaagggaccaaggtggaaatcaaacgg

SEQ ID NO: 684 - DNA, 324 bp, Homo sapiens
gacatccagatgacccagtctccatcctccctgtctgcatctgtaggagaccgtgtcaccatcacttgccgggcaagtcagagtattggtccgtggttaagttggtatcagcagaaaccagggaaagcccctaagctcctgatctatcaggtttcccgtctgcaaagtggggtcccatcacgtttcagtgtcagtggatctgggacagatttcactctcaccatcagcagtctgcaacctgaagattttgctacgtactactgtcaacagaatcttgcgcctccttatacgttcggccaagggaccaaggtggaaatcaaacgg

SEQ ID NO: 685 - protein, 108 aa, Homo sapiens
  VARIANT 108: Xaa = Any Amino Acid
DIQMTQSPSSLSASVGDRVTITCRASQEIHDYLSWYQQKPGKAPKLLIYLSSRLQSGVPSRFSGSRSGTDFTLTISSLQPEDFATYYCQQYHKYPYTFGQGTKVEIKX

SEQ ID NO: 686 - protein, 107 aa, Homo sapiens
DIQMTQSPSSLSASVGDRVTITCRASQSINVRLIWYQQKPGKDPKLLIYSSSHLQSGVPSRFSGSGSGTDFTLTISSLQPEDFATYYCQQYHYTPFTFGQGTKVEIK

SEQ ID NO: 687 - protein, 108 aa, Homo sapiens
  VARIANT 108: Xaa = Any Amino Acid
DIQMTQSPSSLSASVGDRVTITCRASQPILFSLNWYQQKPGKAPKLLIYSASSLQSGVSSRFSGSGFGTDFTLTISSLQPEDFATYYCQQHHSRPYTFGQGTKVEIKX

SEQ ID NO: 688 - protein, 108 aa, Homo sapiens
  VARIANT 108: Xaa = Any Amino Acid
DIQMTQSPSSLSASVGDRVTITCRASQMIRNYLLWYQQAPGKAPKLLIYNASKLQSGVPSRFSGSGSGTDFTLTISSLQPEDFATYYCQQAHTAPFTFGQGTKVEIKX

SEQ ID NO: 689 - protein, 107 aa, Homo sapiens
DIQMTQSPSSLSASVGDRVTITCRASQYINTLLSWYQQKPGKAPKLLIYAQSRLQSGVPSRFSGSGSGTDFTLTISSLQPEDFATYYCQQFAFRPYTFGQGTKVEIK

SEQ ID NO: 690 - protein, 108 aa, Homo sapiens
  VARIANT 108: Xaa = Any Amino Acid
DIQMTQSPSSLSASVGDRVTITCRASQRIGRYLNWYQQKPGKAPELLIYWVSRLQSGVPSRFSGSGFGTDFTLTISSLQPEDFATYYCQQVHSFPMTFGQGTKVEIKX

SEQ ID NO: 691 - protein, 108 aa, Homo sapiens
  VARIANT 108: Xaa = Any Amino Acid
DIQMTQSPSSLSASVGDRVTITCRASQYIGRHLVWYQQKPGKAPKLLIYFASMLQSGVPSRFSGSGSGTDFTLTISSLQPEDFATYHCQQVHFDPFTFGQGTKVEIKX

SEQ ID NO: 692 - protein, 108 aa, Homo sapiens
  VARIANT 108: Xaa = Any Amino Acid
DIQMTQSPSSLSASVGDRVTITCRASQPIHDYLTWYQQKPGKAPKLLIYLASRLQSGVPSRFSGSGSGTDFTLTISSLQPEDFATYYCQQYHVLPYTFGQGTKVEIKX

SEQ ID NO: 693 - protein, 108 aa, Homo sapiens
  VARIANT 108: Xaa = Any Amino Acid
DIQMTQSPSSLSASVGDRVTITCRASQRISHALRWYQQKPGKAPKLLIYRASALQSGVPSRFSGSGSGTDFTLTISSLQPEDFATYYCQQNRSVPFTFGQGTKVEIKX

SEQ ID NO: 694 - protein, 108 aa, Homo sapiens
  VARIANT 108: Xaa = Any Amino Acid
DIQMTQSPSSLSASVGDRVTITCRASQNIRRYLVWYQQKPGKAPKLLIYNASHLQSGVPSRFSGSGSGTDFTLTISSLQPEDFATYYCQQIYLSPFTFGQGTKVEIKX

SEQ ID NO: 695 - protein, 108 aa, Homo sapiens
  VARIANT 108: Xaa = Any Amino Acid
DIQMTQSPSSLSASVGDRVTITCRASQSIGRYIYWYQQKPGKAPKLLIYNVSYLQSGVPSRFSGSGSGTDFTLTISSLQPEDFATYYCQQCFRGPCTFGQGTKVEIKX

SEQ ID NO: 696 - protein, 108 aa, Homo sapiens
  VARIANT 108: Xaa = Any Amino Acid
DIQMTQSPSSLSASVGDRVTITCRASRPISTSLVWYQQKPGKAPKLLIYNASNLQSGVPSRFSGSGSGTDFTLTISSLQPEDFATYYCQQSQTLPVTFGQGTKVEIKX

SEQ ID NO: 697 - protein, 110 aa, Homo sapiens
DIQMTQSPSSLSASVGDRVTITCRASQHITDQLRWYQKKPGKAPKLLIYSASILQSGVPSRFSGSGSGTDFTLTISSLQPEDFATYYCQQIYIRPGTFGQGTKVEIKAAA

SEQ ID NO: 698 - DNA, 324 bp, Homo sapiens
gacatccagatgacccagtctccatcctccctgtctgcatctgtaggagaccgtgtcaccatcacttgccgggcaagtcaggagattcatgattatttaagttggtaccagcagaaaccagggaaagcccctaagctcctgatctatctgtcttcccgtctgcaaagtggggtcccatcacgtttcagtggcagtagatctgggacagatttcactctcaccatcagcagtctgcaacctgaagattttgctacgtactactgtcaacagtatcataagtatccttatacgttcggccaagggaccaaggtggaaatcaaacgg

SEQ ID NO: 699 - DNA, 324 bp, Homo sapiens; identical to SEQ ID NO: 15

SEQ ID NO: 700 - DNA, 324 bp, Homo sapiens
gacatccagatgacccagtctccatcctccctgtctgcatctgtaggagaccgtgtcaccatcacttgccgggcaagtcagcctattttgtttagtttaaattggtaccagcagaaaccagggaaagcccctaagctcctgatctatagtgcgtcctcgttgcaaagtggggtctcatcacgtttcagtggcagtggatttgggacagatttcactctcaccatcagcagtctgcaacctgaagatttcgctacgtactactgtcaacagcatcattcgcggccttatacgttcggccaagggaccaaggtggaaatcaaacgg

SEQ ID NO: 701 - DNA, 324 bp, Homo sapiens
gacatccagatgacccagtctccatcctccctatctgcatccgtaggagaccgtgtcaccatcacttgccgggcaagtcagatgattaggaattatttactttggtaccagcaggcaccagggaaagcccctaagctcctgatctataatgcttccaagttgcaaagtggggtcccatcacgtttcagtggcagtggatctgggacagattttactctcaccatcagcagtctgcaacctgaagattttgctacgtactactgtcaacaggcgcatactgctccttttacgttcggccaagggaccaaggtggaaatcaaacgg

SEQ ID NO: 702 - DNA, 324 bp, Homo sapiens
gacatccagatgacccagtctccatcctccctgtctgcatctgtaggagaccgtgtcaccatcacttgccgggcaagtcagtatattaatactcttttatcttggtaccagcagaaaccagggaaagcccctaagctcctgatctatgcgcagtcccgtttgcaaagtggggtcccatcacgtttcagtggcagtggatctgggacagatttcactctcaccatcagcagtctgcaacctgaagattttgctacgtactactgtcaacagtttgcttttcgtccttatacgttcggccaagggaccaaggtggaaatcaaacgg

SEQ ID NO: 703 - DNA, 324 bp, Homo sapiens
gacatccagatgacccagtctccatcctccctgtctgcatctgtaggagaccgtgtcaccatcacttgccgggcaagtcagcggattggtaggtatttaaattggtaccagcagaaaccagggaaagcccctgagctcctgatctattgggtttcccggttgcaaagtggggtcccatcacgtttcagtggcagtggatttgggacagatttcactctcaccatcagcagtctgcaacctgaagattttgctacgtactactgtcaacaggtgcatagttttcctatgacgttcggccaagggaccaaggtggaaatcaaacgg

SEQ ID NO: 704 - DNA, 324 bp, Homo sapiens
gacatccagatgacccagtctccatcctccctgtctgcatctgtaggagaccgtgtcaccatcacttgccgggcaagtcagtatattgggcgtcatttagtgtggtaccagcagaaaccagggaaagcccctaagctcctgatctattttgcgtccatgttgcaaagtggggtcccatcacgtttcagtggcagtggatctgggacagatttcactctcaccatcagcagtctgcaacctgaagattttgctacgtaccactgtcaacaggttcattttgatccttttacgttcggccaagggaccaaggtggaaatcaaacgg

SEQ ID NO: 705 - DNA, 324 bp, Homo sapiens
gacatccagatgacccagtctccatcctccctgtctgcatctgtaggagaccgtgtcaccatcacttgccgggcaagtcagccgattcatgattatttaacttggtatcagcagaaaccagggaaagcccctaagctcctgatctatttggcgtcccgtttgcaaagtggggtcccatcacgtttcagtggcagtggatctgggacagatttcactctcaccatcagcagtctgcaacctgaagatttcgctacgtactactgtcaacagtatcatgtgctgccttatacgttcggccaagggaccaaggtggaaatcaaacgg

SEQ ID NO: 706 - DNA, 324 bp, Homo sapiens
gacatccagatgacccagtctccatcctccctgtctgcatctgtaggagaccgtgtcaccatcacttgccgggcaagtcagcggattagtcatgcgttacggtggtatcagcagaaaccagggaaagcccctaagctcctgatctatcgtgcttccgctttgcaaagtggggtcccatcacgtttcagtggcagtggatctgggacagatttcactctcaccatcagcagtctgcaacctgaagattttgctacgtactactgtcaacagaatcgttcggtgccttttacgttcggccaagggaccaaggtggaaatcagacgg

SEQ ID NO: 707 - DNA, 324 bp, Homo sapiens
gacatccagatgacccagtctccatcctccctgtctgcatctgtaggagaccgtgtcaccatcacttgccgggcaagtcagaatattcgtaggtatttagtttggtatcagcagaaaccagggaaagcccctaagctcctgatctataatgcgtcccatttgcaaagtggggtcccatcacgtttcagtggcagtggatctgggacagatttcactctcaccatcagcagtctgcaacctgaagattttgctacgtactactgtcaacagatttatctttctccttttacgttcggccaagggaccaaggtggaaatcaaacgg

SEQ ID NO: 708 - DNA, 324 bp, Homo sapiens
gacatccagatgacccagtctccatcctccctgtctgcatctgtaggagaccgtgtcaccatcacttgccgggcaagtcagagtattgggcgttatatatattggtaccagcagaaaccagggaaagcccctaagctcctgatctataatgtttcctatttgcaaagtggggtcccatcacgtttcagtggcagtggatctgggacagatttcactctcaccatcagcagtctgcaacctgaagattttgctacgtactactgtcaacagtgttttcgggggccttgtacgttcggccaagggaccaaggtggaaatcaaacgg

SEQ ID NO: 709 - DNA, 324 bp, Homo sapiens
gacatccagatgacccagtctccatcctccctgtctgcatctgtaggagaccgtgtcaccatcacttgccgggcaagtcggccgatttctactagtttagtttggtaccagcagaaaccagggaaagcccctaagctcctgatctataatgcgtccaatttgcaaagtggggtcccatcacgtttcagtggcagtggatctgggacagatttcactctcaccatcagcagtctgcaacctgaagattttgctacgtactactgtcaacagtcgcagactcttcctgttacgttcggccaagggaccaaggtggaaatcaaacgg

SEQ ID NO: 710 - protein, 108 aa, Homo sapiens
DIQMTQSPSSLSASVGDRVTITCRASQNIDSRLSWYQQKPGKAPKLLIYRTSVLQSGVPSRFSGSGSGTDFTLTISSLQPEDFATYYCQQWDMFPLTFGQGTKVEVKR

SEQ ID NO: 711 - DNA, 324 bp, Homo sapiens
gacatccagatgacccagtctccatcctccctgtctgcatctgtaggagaccgtgtcaccatcacttgccgggcaagtcagaatattgattctcgtttaagttggtaccagcagaaaccagggaaagcccctaagctcctgatctataggacgtccgttttgcaaagtggggtcccatcacgtttcagtggcagtggatctgggacagatttcactctcaccatcagcagtctgcaacctgaagattttgctacgtactactgtcaacagtgggatatgtttcctttgacgttcggccaagggaccaaggtggaagtcaaacgg